(19) United States
(12) Patent Application Publication
(10) Pub. No.: US 2025/0101024 A1
(43) Pub. Date: Mar. 27, 2025

(54) NITROGEN-CONTAINING COMPOUND, ORGANIC ELECTROLUMINESCENT DEVICE, AND ELECTRONIC APPARATUS

(71) Applicant: SHAANXI LIGHTE OPTOELECTRONICS MATERIAL CO. , LTD., Xi'an, Shaanxi (CN)

(72) Inventors: Xianbin Xu, Xi'an, Shaanxi (CN); Lei YANG, Xi'an, Shaanxi (CN)

(21) Appl. No.: 18/552,363

(22) PCT Filed: Feb. 27, 2023
(86) PCT No.: PCT/CN2023/078547

§ 371 Date: Sep. 25, 2023

(30) Foreign Application Priority Data
Jun. 24, 2022 (CN) 202210722794.7

(51) Int. Cl. C07D 487/04; C07B 59/00; C07D 519/00; C09K 11/06; H10K 85/60

(52) U.S. Cl. CPC C07D 487/04; C07B 59/004; C07D 519/00; C09K 11/06; H10K 85/622; H10K 85/654; H10K 85/6572; H10K 85/6574; H10K 85/6576

(57) ABSTRACT

The application relates to the technical field of organic electroluminescent materials, and provides a nitrogen-containing compound, and an organic electroluminescent device and an electronic apparatus including the nitrogen-containing compound. The compound of the application has a core structure that is an indolocarbazolyl group fused with cyclohexane. The compound, when used as a host material of an light emitting layer, can improve balance of charge carriers in the light emitting layer, expand a region where the carriers recombine, improve generation and utilization efficiency of excitons as well as luminescence efficiency and service life of the device.

### CROSS-REFERENCE TO RELATED APPLICATION

The present application is a U.S. National Stage application of International Application No. PCT/CN2023/078547, filed on Feb. 27, 2023, which claims the priority of Chinese patent application CN202210722794.7, filed on Jun. 24, 2022, both of which are incorporated herein by reference in their entireties as a part of the present application.

### FIELD OF THE INVENTION

The present application relates to the technical field of organic electroluminescent materials, and in particular to a nitrogen-containing compound, and an organic electroluminescent device and an electronic apparatus comprising the nitrogen-containing compound.

### BACKGROUND OF THE INVENTION

With the development of electronic technology and the advancement of material science, electronic devices used to achieve electroluminescence or photoelectric conversion have found an increasingly wide range of applications. An organic electroluminescent device (OLED) typically includes a cathode and an anode that are disposed facing each other, and a functional layer disposed between the cathode and the anode. The functional layer consists of a plurality of organic or inorganic film layers, and generally includes an organic light emitting layer, a hole transport layer, an electron transport layer, etc. When a voltage is applied to the cathode and the anode, an electric field is formed between the two electrodes. Under the influence of the electric field, electrons on the cathode side migrate to the light emitting layer, and holes on the anode side also migrate to the light emitting layer. The electrons and the holes recombine in the light emitting layer, forming excitons. The excitons in excited states release energy, causing the light emitting layer to emit light to the outside.

Main problems with existing organic electroluminescent device lie in their service life and efficiency. As display screens become larger and larger, driving voltages are increased accordingly, which necessitates improvement in luminescence efficiency and current efficiency. It is therefore necessary to continue to develop new materials to further improve performance of organic electroluminescent device.

### SUMMARY OF THE INVENTION

Directed against the above problems with the existing technology, the present application aims at providing a nitrogen-containing compound, and an electronic device and an electronic apparatus comprising the nitrogen-containing compound. The nitrogen-containing compound can be used in an organic electroluminescent device to improve performance of the device. A first aspect of the present application provides a nitrogen-containing compound having a structure formed by fusing a structure of the following Formula 1 and a structure of the following Formula 2 together, wherein the structure of Formula 2 is fused via * to positions of any two adjacent * on Ring B in the structure of Formula 1;

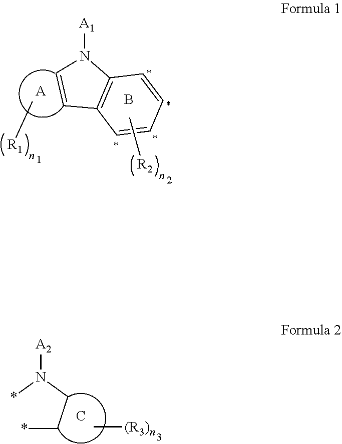

Ring A is benzocyclohexane;
Ring C is selected from an C₆₋₁₄ aromatic ring;
groups A₁ and A₂ are each independently selected from a structure shown in Formula a-1 or a structure shown in Formula a-2, and at least one of A₁ and A₂ is the structure shown in Formula a-1;

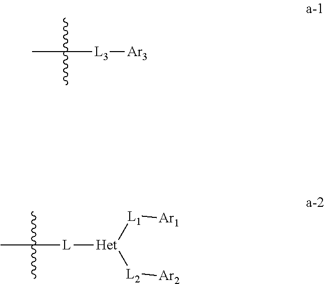

L, L₁, L₂ and L₃ are identical or different, and are each independently selected from a single bond, a substituted or unsubstituted C₆₋₃₀ arylene, or a substituted or unsubstituted C₃₋₃₀ heteroarylene;
Ar₃ is selected from a substituted or unsubstituted C₆₋₄₀ aryl, or a substituted or unsubstituted C₃₋₄₀ heteroaryl;
Het is a C₃₋₂₀ nitrogen-containing heteroarylene;
Ar₁ and Ar₂ are identical or different, and are each independently selected from hydrogen, a substituted or unsubstituted C₆₋₄₀ aryl, or a substituted or unsubstituted C₃₋₄₀ heteroaryl;
each R₁, each R₂ and each R₃ are identical or different, and are each independently selected from deuterium, a cyano, a halogen, an C₁₋₁₀ alkyl, a C₁₋₁₀ haloalkyl, a C₁₋₁₀ deuterated alkyl, a C₃₋₁₂ trialkylsilyl, a triphenylsilyl, an C₆₋₂₀ aryl, a C₆₋₂₀ deuterated aryl, a C₆₋₂₀ halogenated aryl, a C₃₋₂₀ heteroaryl, or a C₃₋₁₀ cycloalkyl; and optionally, any two adjacent R₂s form a 6-membered to 14-membered aromatic ring, the 6-membered to 14-membered aromatic ring being optionally substituted by zero, one, two, three, four, five or six R₄s;
each R₄ is independently selected from deuterium, a cyano, a halogen, an C₁₋₁₀ alkyl, a C₁₋₁₀ haloalkyl, a C₁₋₁₀ deuterated alkyl, or a C₃₋₁₂ trialkylsilyl;
n₁ is selected from 1, 2, 3, 4, 5, 6, 7, 8, 9 or 10;
n₂ is selected from 0, 1 or 2;
n₃ is selected from 0, 1, 2, 3, 4, 5, 6, 7 or 8;
substituents of L, L₁, L₂, L₃, Ar₁, Ar₂ and Ar₃ are identical or different, and are each independently selected from deuterium, a cyano, a halogen, an C₁₋₁₀ alkyl, a C₁₋₁₀ haloalkyl, a C₁₋₁₀ deuterated alkyl, a C₃₋₁₂ trialkylsilyl, a triphenylsilyl, an C₆₋₂₀ aryl, a C₆₋₂₀ deuterated aryl, a C₆₋₂₀ halogenated aryl, a C₃₋₂₀ heteroaryl or a C₃₋₁₀ cycloalkyl; and optionally, any two adjacent substituents may form a saturated or unsaturated 3-membered to 15-membered ring.

A second aspect of the present application provides an organic electroluminescent device comprising an anode and a cathode that are disposed facing each other, and a functional layer disposed between the anode and the cathode. The functional layer comprises the nitrogen-containing compound described above.

A third aspect of the present application provides an electronic apparatus comprising the organic electroluminescent device described in the second aspect.

The compound of the present application has a core structure that is an indolocarbazolyl group fused with cyclohexane, the indolocarbazolyl group fused with cyclohexane being further linked with an aryl or heteroaryl group via two nitrogen atoms in the indolocarbazolyl group. The indolocarbazolyl group has an excellent hole transport ability, and when fused with cyclohexane, can further enhance the hole mobility of the carbazolyl group as a result of the hyperconjugation effect, thereby giving the compound of the present application an even better hole mobility. In addition, substituents are added to cyclohexane of the parent structure; these substituents, in spatial configuration, are outside the conjugate plane of the carbazolyl group, and can thus form a certain degree of steric hindrance, which can result in fine regulation of intermolecular accumulation in the compound, and make it possible to form the compound into better amorphous films. The use of the compound of the present application as a host material of an light emitting layer can improve balance of charge carriers in the light emitting layer, expand a region where the carriers recombine, improve generation and utilization efficiency of excitons as well as luminescence efficiency and service life of a device.

### BRIEF DESCRIPTION OF THE DRAWINGS

The accompanying drawings are intended to provide a further understanding of the present application and form a part of the specification. The accompanying drawings, together with the following embodiments, are used to illustrate the present application, but do not constitute any limitation to the present application.

FIG. 1 is a schematic structural diagram of an organic electroluminescent device according to an embodiment of the present application.

FIG. 2 is a schematic structural diagram of an electronic apparatus according to an embodiment of the present application.

List of Reference Signs

| | | | |
|---|---|---|---|
| 100: anode | 200: cathode | 300: functional | 310: hole injection |
| | | layer | layer |

| | |
|---|---|
| 321: first hole transport layer | 322: second hole transport layer |
| 330: organic light emitting layer | 340: electron transport layer |
| 350: electron injection layer | 400: electronic apparatus |

### DETAILED DESCRIPTION OF THE EMBODIMENTS

Exemplary embodiments will now be described more comprehensively with reference to the accompanying drawings. The exemplary embodiments, however, can be implemented in a variety of forms and should not be interpreted as being limited to the examples set forth herein. On the contrary, these embodiments are provided to make the present application more comprehensive and complete, and to communicate the concepts of these exemplary embodiments fully to those skilled in the art. Features, structures, or characteristics described can be combined in one or more embodiments in any suitable manner. In the following description, many specific details are provided to give a full understanding of the embodiments of the present application.

In a first aspect, the present application provides a nitrogen-containing compound having a structure formed by fusing a structure of the following Formula 1 and a structure of the following Formula 2 together, wherein the structure of Formula 2 is fused via * to positions of any two adjacent * on Ring B in the structure of Formula 1;

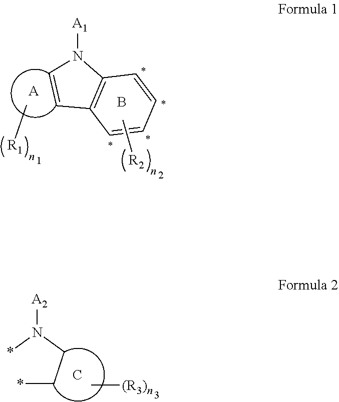

Ring A is benzocyclohexane;
Ring C is selected from an C₆₋₁₄ aromatic ring;
groups A₁ and A₂ are each independently selected from a structure shown in Formula a-1 or a structure shown in Formula a-2, and at least one of A₁ and A₂ is the structure shown in Formula a-1;

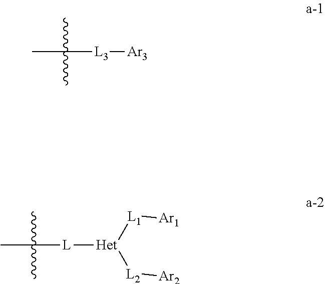

L, L₁, L₂ and L₃ are identical or different, and are each independently selected from a single bond, a substituted or unsubstituted C₆₋₃₀ arylene, or a substituted or unsubstituted C₃₋₃₀ heteroarylene;
Ar₃ is selected from a substituted or unsubstituted C₆₋₄₀ aryl, or a substituted or unsubstituted C₃₋₄₀ heteroaryl;
Het is a C₃₋₂₀ nitrogen-containing heteroarylene;
Ar₁ and Ar₂ are identical or different, and are each independently selected from hydrogen, a substituted or unsubstituted C₆₋₄₀ aryl, or a substituted or unsubstituted C₃₋₄₀ heteroaryl;
each R₁, each R₂ and each R₃ are identical or different, and are each independently selected from deuterium, a cyano, a halogen, an C₁₋₁₀ alkyl, a C₁₋₁₀ haloalkyl, a C₁₋₁₀ deuterated alkyl, a C₃₋₁₂ trialkylsilyl, a triphenylsilyl, an C₆₋₂₀ aryl, a C₆₋₂₀ deuterated aryl, a C₆₋₂₀ halogenated aryl, a C₃₋₂₀ heteroaryl or a C₃₋₁₀ cycloalkyl; and optionally, any two adjacent R₂s form a 6-membered to 14-membered aromatic ring, the 6-membered to 14-membered aromatic ring being optionally substituted by zero, one, two, three, four, five or six R₄s;
each R₄ is independently selected from deuterium, a cyano, a halogen, an C₁₋₁₀ alkyl, a C₁₋₁₀ haloalkyl, a C₁₋₁₀ deuterated alkyl, or a C₃₋₁₂ trialkylsilyl;
n₁ is selected from 1, 2, 3, 4, 5, 6, 7, 8, 9 or 10;
n₂ is selected from 0, 1 or 2;
n₃ is selected from 0, 1, 2, 3, 4, 5, 6, 7 or 8;
substituents of L, L₁, L₂, L₃, Ar₁, Ar₂ and Ar₃ are identical or different, and are each independently selected from deuterium, a cyano, a halogen, an C₁₋₁₀ alkyl, a C₁₋₁₀ haloalkyl, a C₁₋₁₀ deuterated alkyl, a C₃₋₁₂ trialkylsilyl, a triphenylsilyl, an C₆₋₂₀ aryl, a C₆₋₂₀ deuterated aryl, a C₆₋₂₀ halogenated aryl, a C₃₋₂₀ heteroaryl or a C₃₋₁₀ cycloalkyl; and optionally, any two adjacent substituents may form a saturated or unsaturated 3-membered to 15-membered ring.

The nitrogen-containing compound provided in the present application does not include the following compounds:

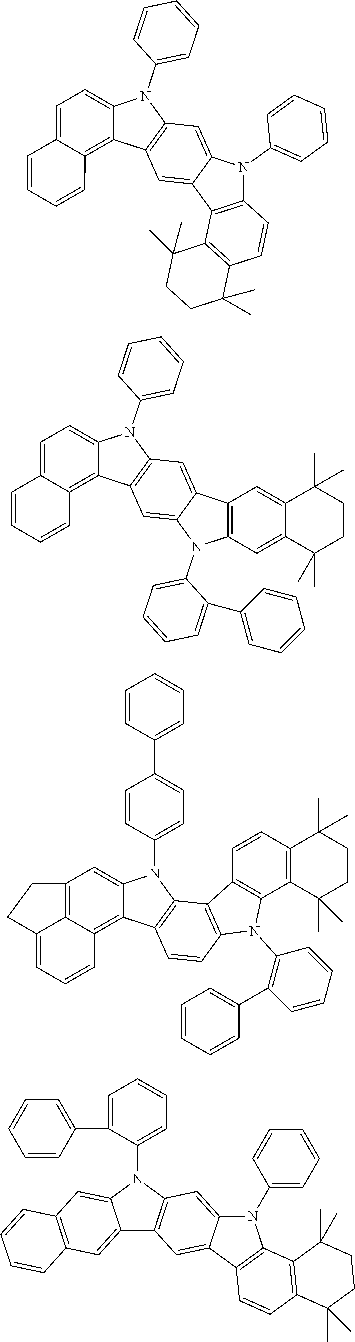

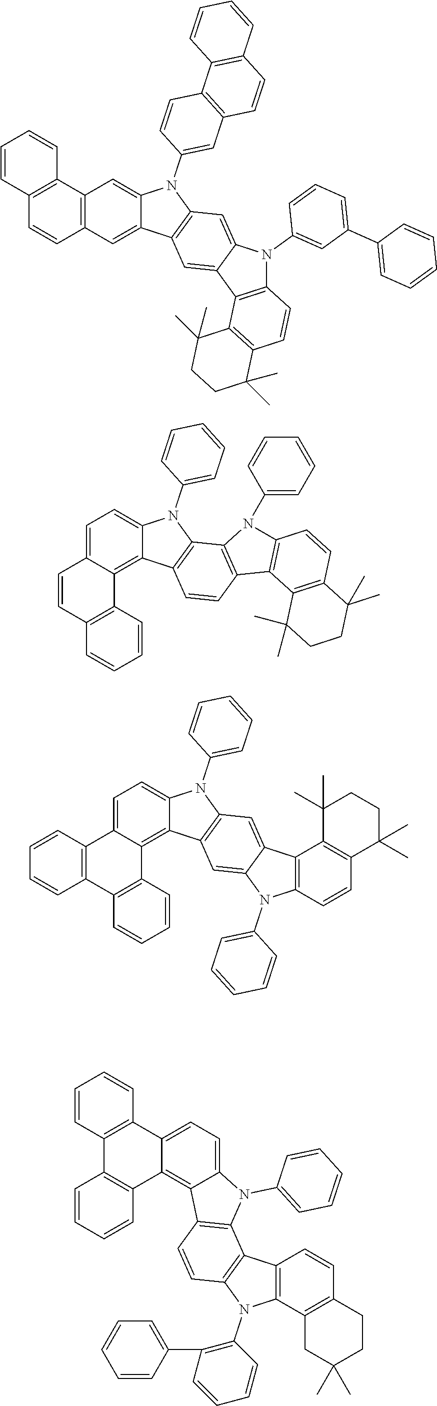

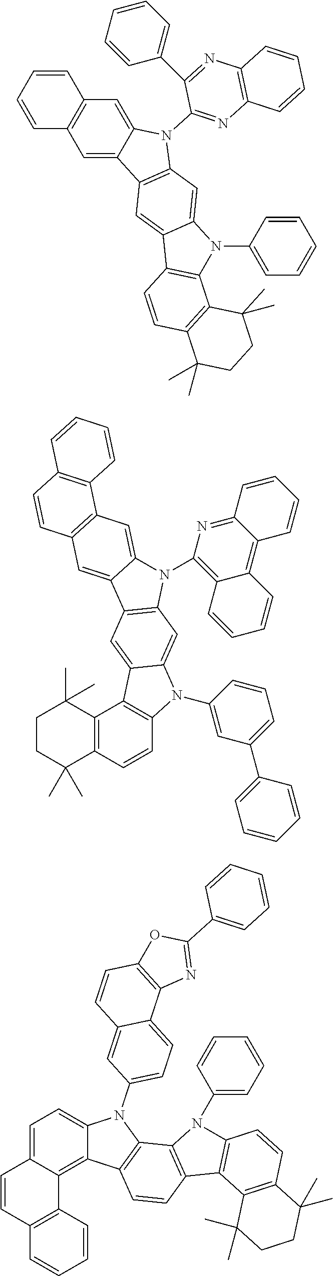

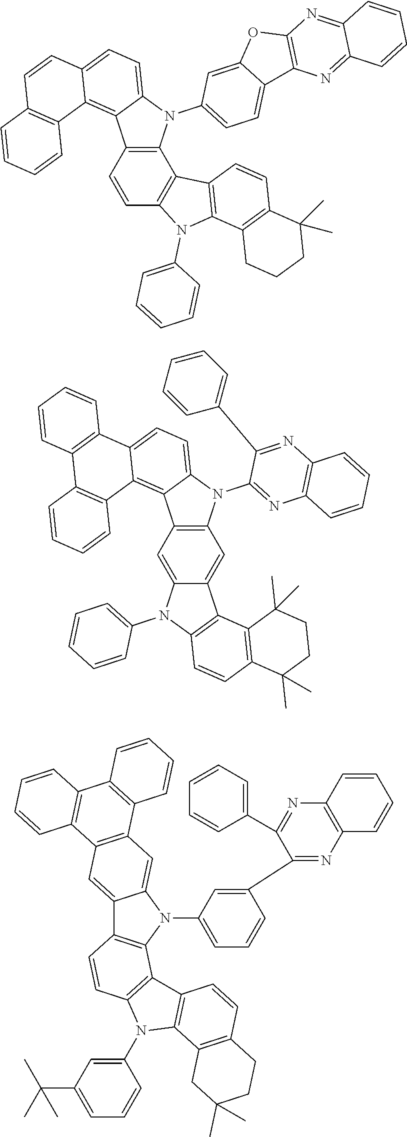

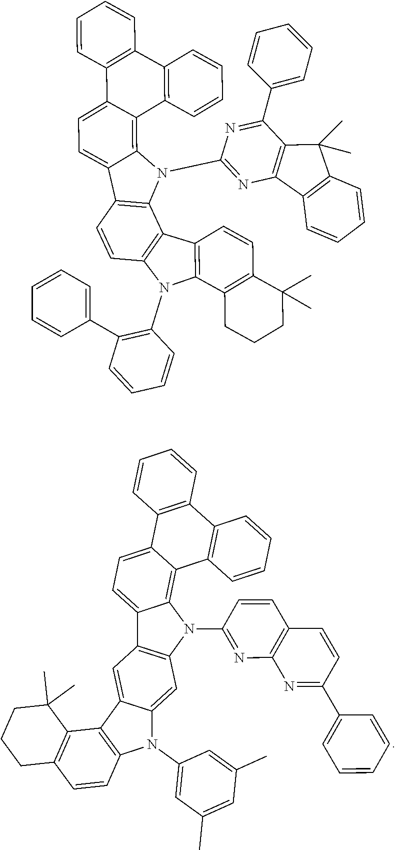 [SMILES: CC1(C)CCCc2c1ccc1c3c(ccc4c5ccc6c7ccccc7c7ccccc7c6c5n(-c5nc(-c6ccccc6)c6c(n5)-c5ccccc5C6(C)C)c43)n(-c3ccccc3-c3ccccc3)c21 ; Cc1cc(C)cc(-n2c3cc4c(cc3c3c5c(ccc32)CCCC5(C)C)c2ccc3c5ccccc5c5ccccc5c3c2n4-c2ccc3ccc(-c4ccccc4)nc3n2)c1]

In the present application, the term “optional” or “optionally” means that the subsequently described event or circumstance may or may not occur. For example, “optionally, any two adjacent substituents form a ring” means that the two substituents may or may not form a ring, i.e., including instances where the two adjacent substituents form a ring and instances where the two adjacent substituents do not form a ring. For example, “optionally, among L, L₁, L₂, L₃, Ar₁, Ar₂ and Ar₃, any two adjacent substituents form a ring” means that any two adjacent substituents among L, L₁, L₂, L₃, Ar₁, Ar₂ and Ar₃ are linked to each other to form a ring, or any two adjacent substituents among L, L₁, L₂, L₃, Ar₁, Ar₂ and Ar₃ may exist independently. The mentioning of “any two adjacent” may involve instances where there are two substituents on a same atom and also instances where there is one substituent on each of two adjacent atoms. When there are two substituents on a same atom, the two substituents, together with the atom to which they are attached, may form a saturated or unsaturated spiro ring; and when there is one substituent on each of two adjacent atoms, the two substituents may be fused into a ring.

In the present application, the expression “each . . . independently”, may be used interchangeably with the expressions “ . . . independently”, and “ . . . each independently”, and all these expressions should be interpreted in a broad sense. They can not only mean that, in different groups, specific options expressed between the same symbols do not affect each other, but also mean that in a same group, specific options expressed between the same symbols do not affect each other. For example, the meaning of

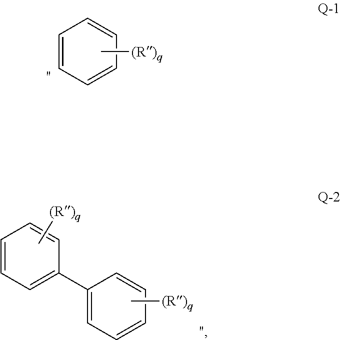

where each q is independently 0, 1, 2 or 3, and each R″ is independently selected from hydrogen, deuterium, fluorine or chlorine” is as follows: Formula Q-1 represents that q substituent R″s exist on a benzene ring, each R″ can be the identical or different, and options of each R″ do not affect each other; and Formula Q-2 represents that each benzene ring of biphenyl has q substituent R″s, the number q of the substituent R″s on the two benzene rings can be the identical or different, each R″ can be the identical or different, and options of each R″ do not affect each other.

In the present application, the term “substituted or unsubstituted” means that the functional group defined by the term may or may not have a substituent (hereinafter referred to as Rc for ease of description). For example, “a substituted or unsubstituted aryl” refers to an aryl group with a substituent Rc or an aryl group without a substituent. The above-mentioned substituents, namely Rc, may be, for example, deuterium, halogen, cyano, heteroaryl, aryl, trialkylsilyl, triphenylsilyl, alkyl, haloalkyl, deuterated alkyl, deuterated aryl, halogenated aryl, cycloalkyl, etc. There may be one or more substituents.

In the present application, the term “more” in “one or more” means two or more than two, for example, two, three, four, five, six, etc.

Hydrogen atoms in the structure of the compound of the present application include various isotopic atoms of hydrogen elements, such as hydrogen (H), deuterium (D), or tritium (T).

In the present application, the number of carbon atoms of a substituted or unsubstituted functional group refers to the number of all carbon atoms. For example, if L is a substituted C₁₂ arylene, then the number of all carbon atoms of the arylene and of substituents thereof is 12.

In the present application, an aryl refers to any functional group or substituent derived from an aromatic carbon ring. An aryl may be a monocyclic aryl (e.g., phenyl) or a polycyclic aryl. In other words, an aryl may be a monocyclic aryl, a fused cycloaryl, two or more monocyclic aryls linked by carbon-carbon single bond conjugation, a monocyclic aryl and a fused cycloaryl linked by carbon-carbon single bond conjugation, two or more fused cycloaryls linked by carbon-carbon single bond conjugation. That is, unless otherwise specified, two or more aromatic groups linked by carbon-carbon single bond conjugation may also be regarded as an aryl of the present application. Among them, fused cycloaryl may include, for example, bicyclic fused cycloaryl (e.g., naphthyl), tricyclic fused aryl (e.g., phenanthryl, fluorenyl, anthryl) and the like. An aryl does not contain heteroatoms such as B, N, O, S, P, Se, and Si. Examples of aryls may include, but are not limited to, phenyl, naphthyl, fluorenyl, phenyl-naphthyl, spirodifluorenyl, anthryl, phenanthryl, biphenyl, terphenyl, triphenylene

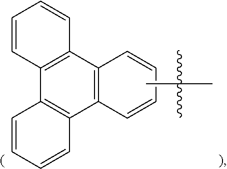

perylenyl, benzo[9,10]phenanthryl, pyrenyl, benzofluoranthryl, chrysenyl, etc.

In the present application, an arylene refers to a divalent or polyvalent group formed by further removing one or more hydrogen atoms from an aryl.

In the present application, terphenyl include

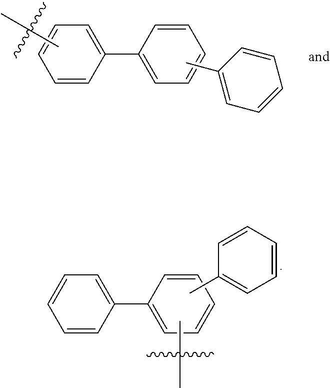

In the present application, the number of carbon atoms of a substituted aryl is the number of all carbon atoms of the aryl and substituents of the aryl. For example, a substituted C₁₈ aryl means that the number of all carbon atoms of the aryl and of substituents thereof is 18.

In the present application, the number of carbon atoms of a substituted or unsubstituted aryl (arylene) may be 6, 8, 10, 12, 13, 14, 15, 16, 17, 18, 19, 20, 21, 22, 23, 24, 25, 26, 28, 30, 31, 33, 34, 35, 36, 38 or 40. In some embodiments, a substituted or unsubstituted aryl is a substituted or unsubstituted C₆₋₄₀ aryl. In other embodiments, a substituted or unsubstituted aryl is a substituted or unsubstituted C₆₋₃₀ aryl. In other embodiments, a substituted or unsubstituted aryl is a substituted or unsubstituted C₆₋₂₅ aryl. In other embodiments, a substituted or unsubstituted aryl is a substituted or unsubstituted C₆₋₁₅ aryl.

In the present application, a fluorenyl may be substituted by one or more substituents. In the case where the above fluorenyl is substituted the substituted fluorenyl may be, but is not limited

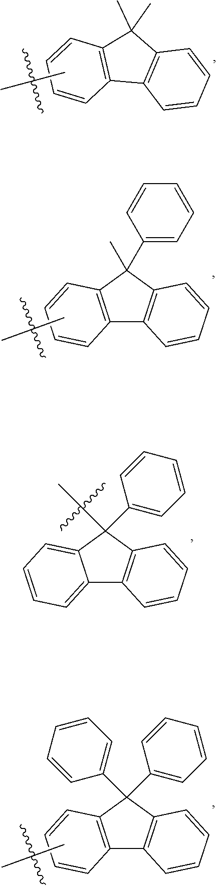

and the like.

In the present application, aryls, as substituents of L₁, L₂, L₃, Ar₁, Ar₂ and Ar₃, may be, but are not limited to, phenyl, naphthyl, phenanthryl, biphenyl, fluorenyl, dimethylfluorenyl, etc.

In the present application, a 6-membered to 14-membered aromatic ring refer to an aromatic ring having 6-membered to 14 ring atoms, such as, but not limited to, a benzene ring, a naphthalene ring, an anthracene ring, or a phenanthrene ring.

In the present application, a heteroaryl refers to a monovalent aromatic ring containing 1, 2, 3, 4, 5 or 6 heteroatoms or a derivative thereof. The heteroatoms may be one or more selected from B, O, N, P, Si, Se and S. A heteroaryl may be a monocyclic heteroaryl or polycyclic heteroaryl. In other words, a heteroaryl may be a single aromatic ring system, or a plurality of aromatic ring systems linked by carbon-carbon single-bond conjugation, with any one of the aromatic ring systems being an aromatic monocyclic ring or a fused aromatic ring. For example, heteroaryl may include, but are not limited to, thiophenyl, furyl, pyrryl, imidazolyl, thiazolyl, oxazolyl, oxadiazolyl, triazolyl, pyridinyl, bipyridinyl, pyrimidyl, triazinyl, acridinyl, pyridazinyl, pyrazinyl, quinolyl, quinazolinyl, quinoxalinyl, phenoxazinyl, phthalazinyl, pyridopyrimidinyl, pyridopyrazinyl, pyrazinopyrazinyl, isoquinolyl, indolyl, carbazolyl, benzoxazolyl, benzimidazolyl, benzothiazolyl, benzocarbazolyl, benzothiophenyl, dibenzothiophenyl, thienothiophenyl, benzofuranoyl, phenanthrolinyl, isoxazolinyl, thiadiazolyl, phenothiazinyl, silafluorenyl, dibenzofuranoyl, N-phenylcarbazolyl, N-pyridylcarbazolyl, N-methylcarbazolyl, etc.

In the present application, the heteroarylene involved is a divalent or polyvalent group formed by further removing one or more hydrogen atoms from a heteroaryl.

In the present application, the number of carbon atoms of a substituted or unsubstituted heteroaryl (heteroarylene) may be 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40. In some embodiments, a substituted or unsubstituted heteroaryl is a substituted or unsubstituted C₃₋₄₀ heteroaryl. In other embodiments, a substituted or unsubstituted heteroaryl is a substituted or unsubstituted C₃₋₃₀ heteroaryl. In other embodiments, a substituted or unsubstituted heteroaryl is a substituted or unsubstituted C₅₋₁₂ heteroaryl.

In the present application, heteroaryls, as substituents of L, L₁, L₂, L₃, Ar₁, Ar₂ and Ar₃, may be, but is not limited to, for example, pyridyl, carbazolyl, quinolyl, isoquinolyl, phenanthrolinyl, benzoxazolyl, benzothiazolyl, benzimidazolyl, dibenzothiophenyl or dibenzofuranyl.

In the present application, a substituted heteroaryl may mean that one or more than two hydrogen atoms in the heteroaryl group are substituted by group such as deuterium atom, halogen, —CN(cyano), aryl, heteroaryl, trialkylsilyl, alkyl, cycloalkyl, haloalkyl, etc. It should be appreciated that the number of carbon atoms of the substituted heteroaryl is the number of all carbon atoms of the heteroaryl and the substituents of the heteroaryl.

In the present application, the C₁₋₁₀ alkyls may include C₁₋₁₀ straight-chain alkyls and C₃₋₁₀ branched-chain alkyls. For example, the number of carbon atoms of an alkyl is, for example, 1, 2, 3, 4, 5, 6, 7, 8, 9 or 10, and specific examples of alkyls include, but are not limited to, methyl, ethyl, n-propyl, isopropyl, n-butyl, isobutyl, tert-butyl, n-pentyl, isopentyl, neopentyl, n-hexyl, etc.

In the present application, halogen is, for example, fluorine, chlorine, bromine or iodine.

In the present application, specific example of trialkylsilyl include, but are not limited to, trimethylsilyl, triethylsilyl, etc.

In the present application, specific example of haloalkyl include, but are not limited to, trifluoromethyl.

In the present application, specific example of deuterated alkyl include, but are not limited to, trideuteromethyl.

In the present application, a deuterated aryl is an aryl containing deuterium, such as but not limited to, deuterated phenyl, deuterated naphthyl, deuterated biphenyl, etc.

In the present application, a halogenated aryl refers to an aryl group with a halogen substituent, such as but not limited to, fluorophenyl, fluoronaphthyl, fluorobiphenyl, etc.

In the present application, the number of carbon atoms of a C₃₋₃₀ cycloalkyl is, for example, 3, 4, 5, 6, 7, 8 or 10. Specific examples of cycloalkyl groups include, but are not limited to, cyclopentyl, cyclohexyl, adamantly, etc.

In the present application, a non-orientating linkage bond is a single bond “ [symbol] ”, “ [symbol] ”, and “ [symbol] ” extending from a ring system, and it indicates that the linkage bond can be linked at one end thereof to any position in the ring system through which the bond passes, and linked at the other end thereof to the rest of the compound molecule. For example, as shown in Formula (f) below, the naphthalyl represented by Formula (f) is linked to other positions of the molecule via two non-orientating linkage bonds passing through the two rings, which indicates any of possible linkages shown in Formulae (f-1)-(f-10):

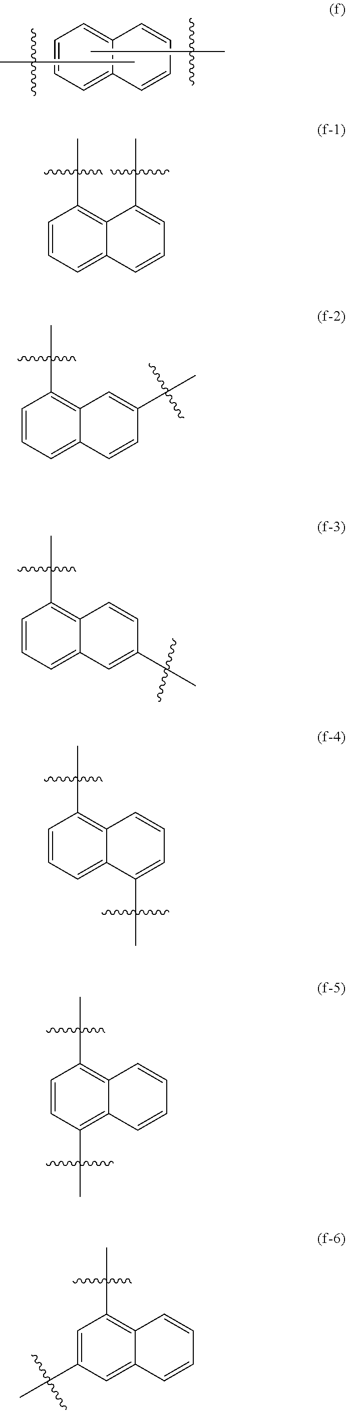

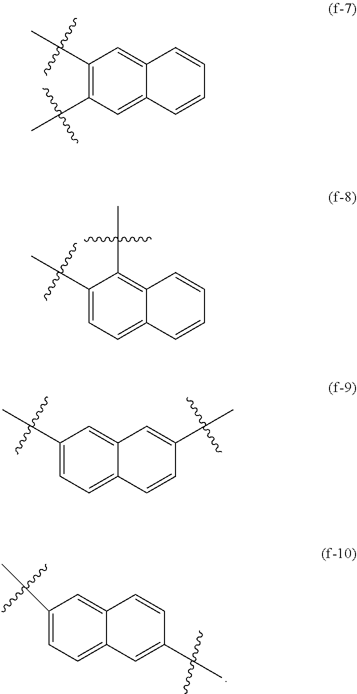 [SMILES: *c1cc2ccccc2cc1* ; *c1ccc2ccccc2c1* ; *c1ccc2ccc(*)cc2c1 ; *c1ccc2cc(*)ccc2c1]

As another example, as shown in Formula (X′) below, the dibenzofuranyl represented by Formula (X′) is linked to other positions of the molecule via a non-orientating linkage bond extending from the middle of a benzene ring on one side, which indicates any of possible linkages shown in Formulae (X′-1)-(X′-4):

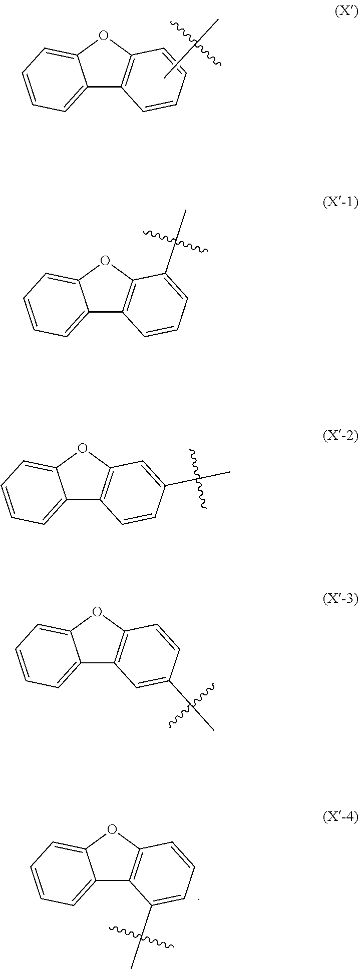

A non-orientating substituent in the present application refers to a substituent linked via a single bond extending from the center of a ring system, and it means that the substituent may be linked to any possible position in the ring system. For example, as shown in Formula (Y) below, the substituent R′ represented by Formula (Y) is linked to a quinoline ring via a non-orientating linkage bond, which indicates any of possible linkages shown in Formulae (Y-1)-(Y-7):

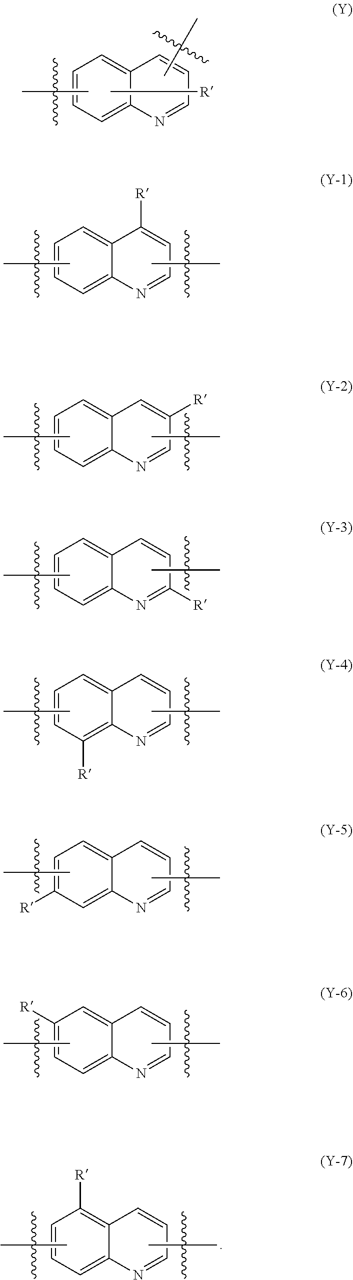

In some embodiments, the structure of Formula 1 has a structure shown in the following Formulae (i-a)-(i-c):

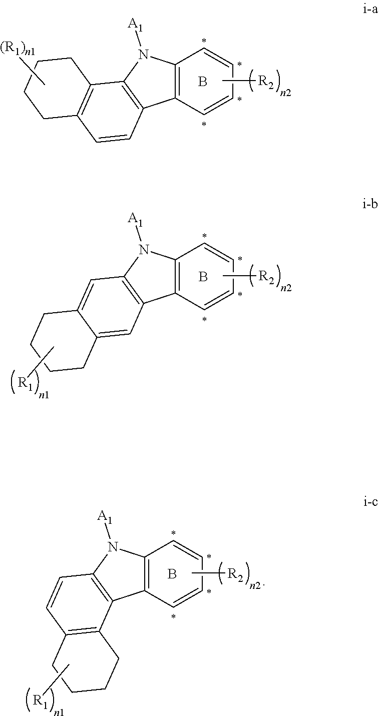

In some embodiments, the structure of Formula 1 has a structure shown in the following Formulae (i-d)-(i-f):

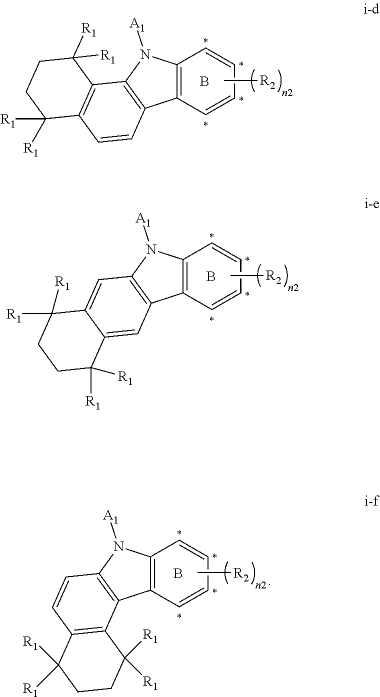

In some embodiments, the structure of Formula 1 is selected from structures shown in Formulae (i-1)-(i-3):

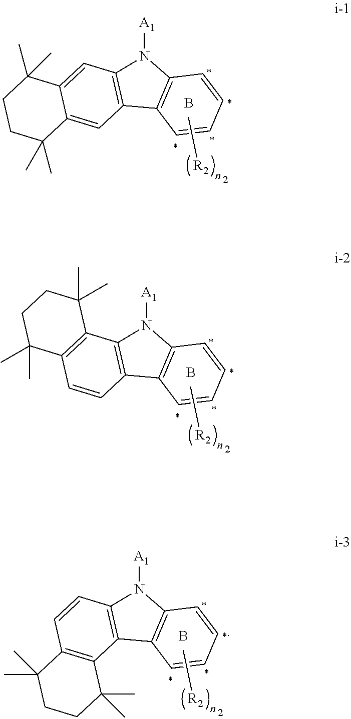

In some embodiments, Ring C is selected from a benzene ring, a naphthalene ring, or a phenanthrene ring.

Alternatively, Ring C is selected from the following structures:

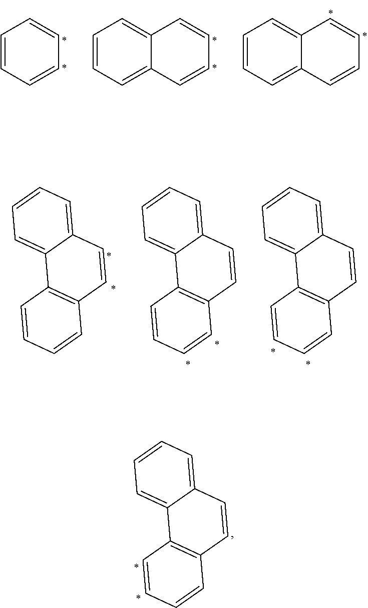

in which Position * indicates fused site on Ring C.

In some embodiments, the structure of Formula 2 has the following structures:

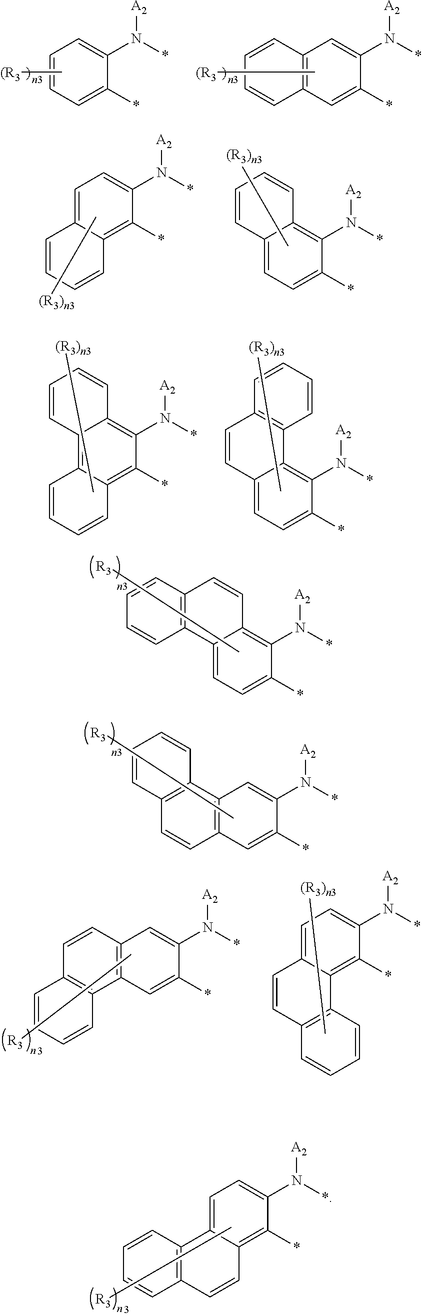

In some embodiments, each R₁, each R₂ and each R₃ are identical or different, and are each independently selected from deuterium, a cyano, a fluorine, a trideuteromethyl, a trimethylsilyl, a trifluoromethyl, a cyclopentyl, a cyclohexyl, an adamantly, a methyl, an ethyl, an isopropyl, a tert-butyl, a phenyl or a naphthyl.

Optionally, any two adjacent R₂s form a benzene ring, the benzene ring being optionally substituted by zero, one, two, three, four, five or six R₄s. Each R₄ is each independently selected from deuterium, a cyano, a fluorine, a trideuteromethyl or a trifluoromethyl.

In some embodiments, the nitrogen-containing compound has structural formulae shown in the following Formulae (S-1)-(S-21):

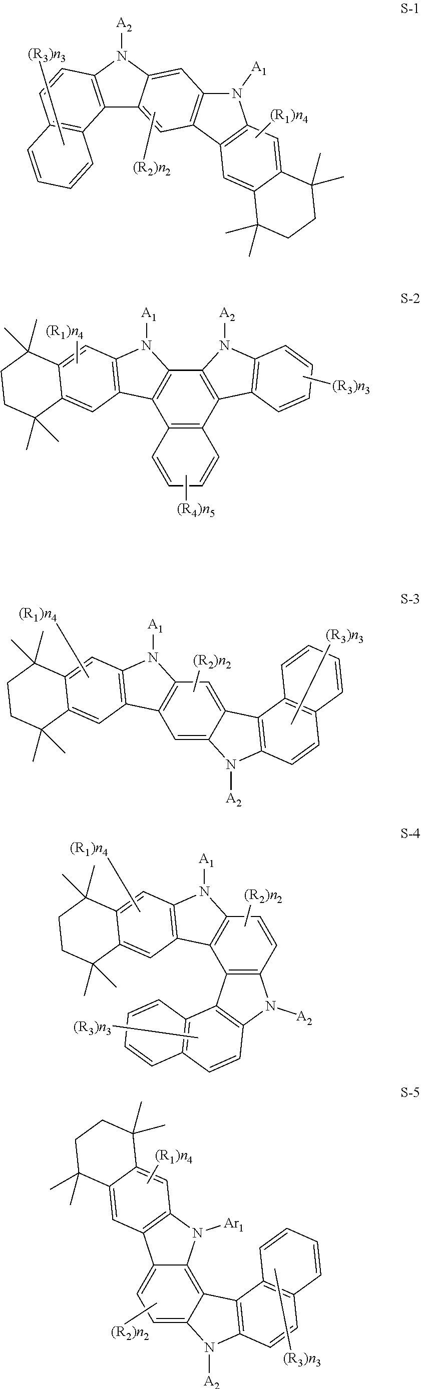

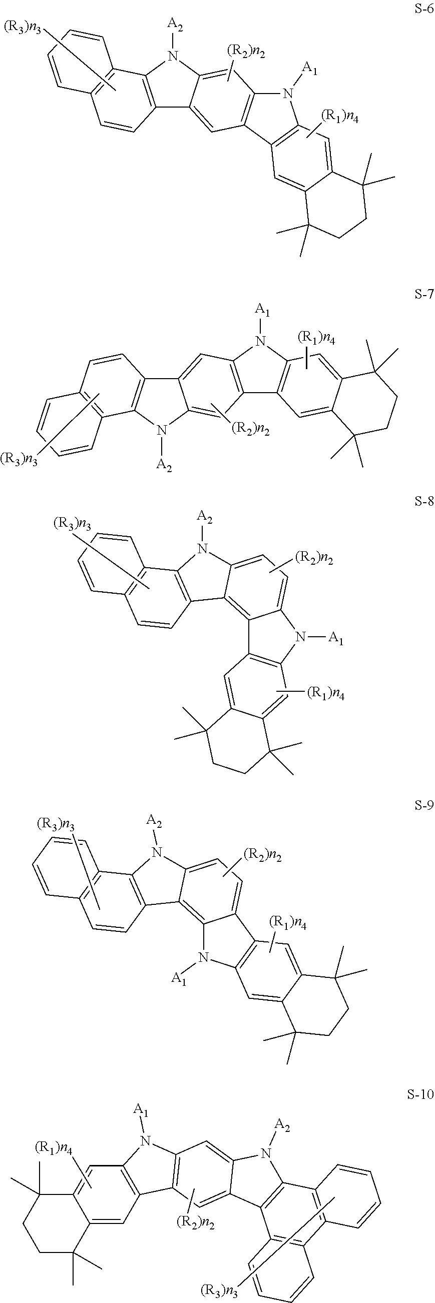

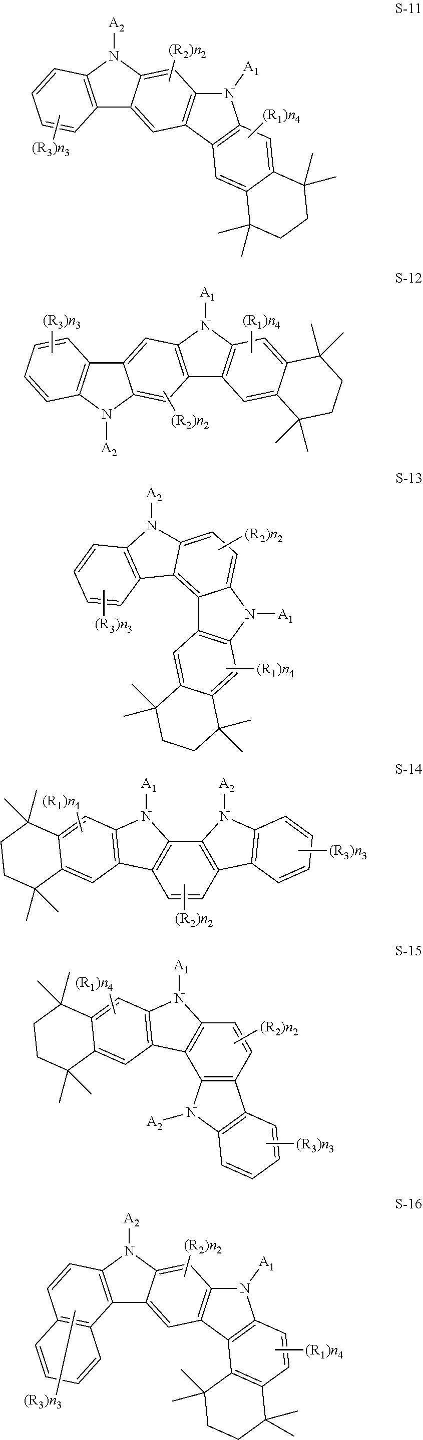

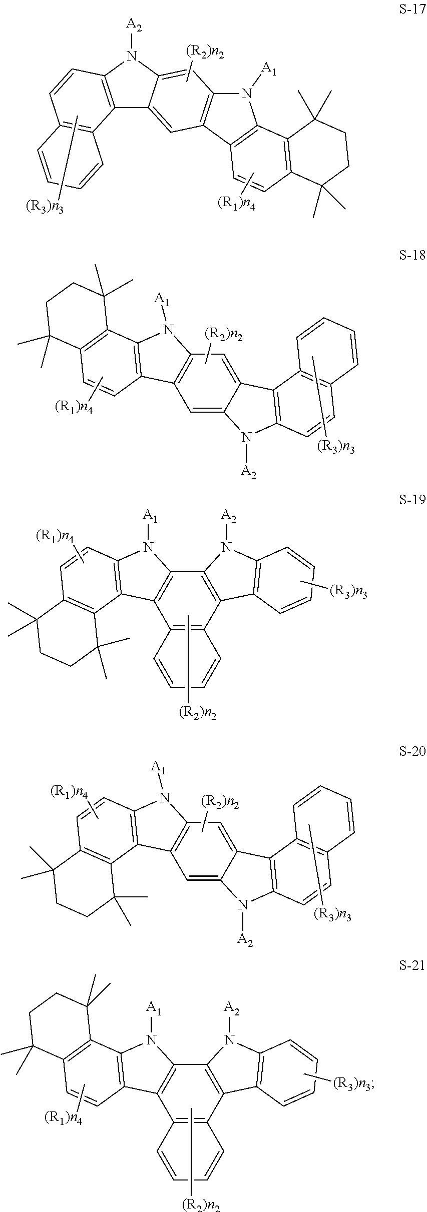

wherein each R₁, each R₂ and each R₃ are identical or different, and are each independently selected from deuterium, a cyano, a fluorine, a trideuteromethyl, a trimethylsilyl, a trifluoromethyl, a cyclopentyl, a cyclohexyl, an adamantly, a methyl, an ethyl, an isopropyl, a tert-butyl, a phenyl or a naphthyl;
each R₄ is independently selected from deuterium, a cyano, a fluorine, a trideuteromethyl or a trifluoromethyl;
n₂ is selected from 0, 1 or 2;
n₃ is selected from 0, 1, 2, 3, 4, 5, 6, 7 or 8;
n₄ is selected from 0, 1 or 2; and
n₅ is selected from 0, 1, 2, 3 or 4.

In some embodiments, both A₁ and A₂ are the structure shown in Formula a-1; alternatively, one of A₁ and A₂ has the structure shown in Formula a-1, and the other has the structure shown in Formula a-2. When both A₁ and A₂ are the structure shown in Formula a-1

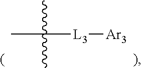

two L₃ are identical or different, and two Ar₃ are identical or different.

The compound of the present application, when its parent structure, a cyclohexane-fused indolocarbazolyl group, is linked with an electron-rich aryl group or heteroaryl group, may form a material with an excellent hole transport property. Further, when cyclohexane is substituted, the substituents, in spatial configuration, are outside the conjugate plane of the indolocarbazolyl group, and can result in a certain degree of steric hindrance, thereby leading to fine regulation of intermolecular accumulation in the compound, and making it possible to form the compound into a better amorphous thin film. When the cyclohexane-fused indolocarbazolyl group is linked with an electron-deficient heteroarylene group (Het) containing at least two nitrogen atoms, the compound may form an electron transport-type host material for an light emitting layer or a bipolar host material for an light emitting layer.

In some embodiments, Het is selected from the following groups:

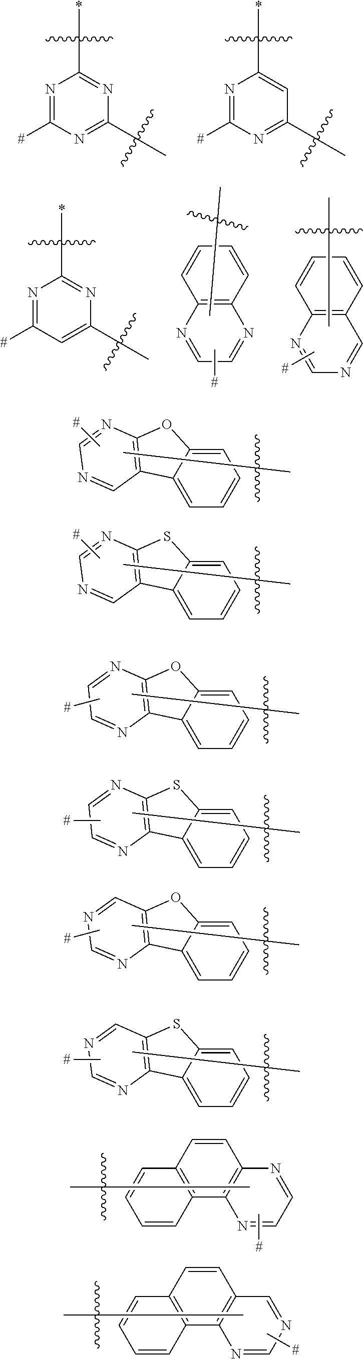

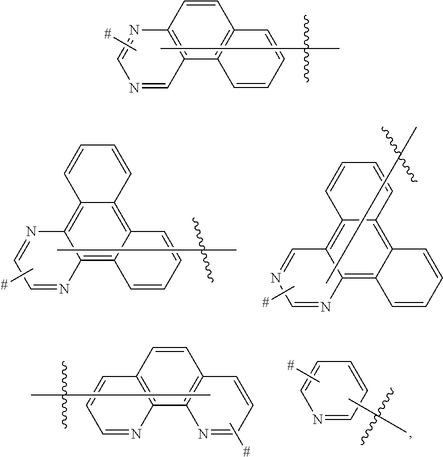

in which: [symbol] represents a bond linked with L; [symbol] represents a bond linked with L₁; [symbol] represents a bond linked with L₂; where a formula contains no [symbol] , it means that, in

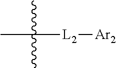

linked at this position, L₂ is a single bond and Ar₂ is hydrogen.

Alternatively, Het is selected from the following groups:

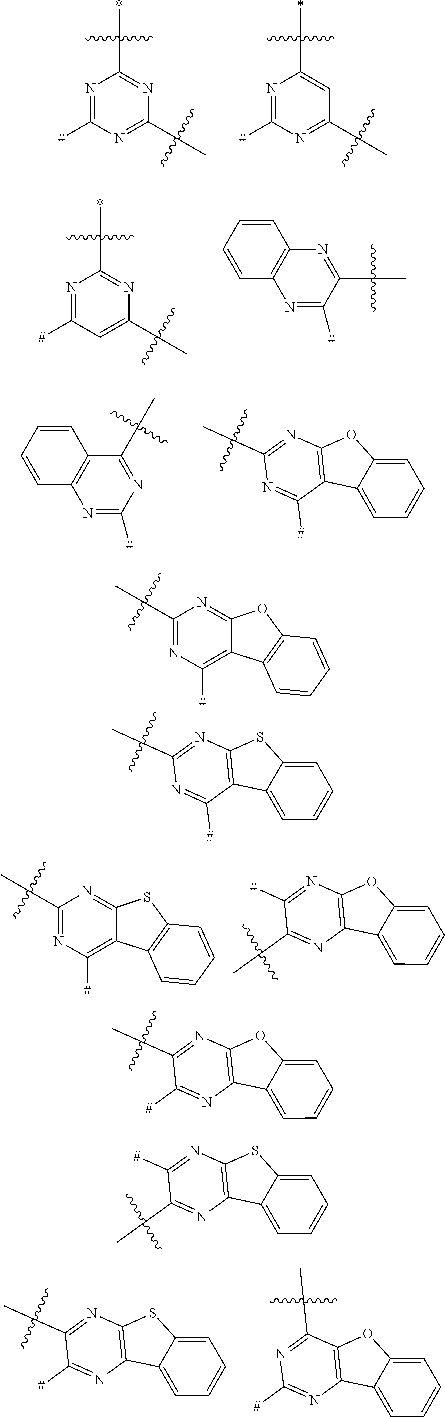

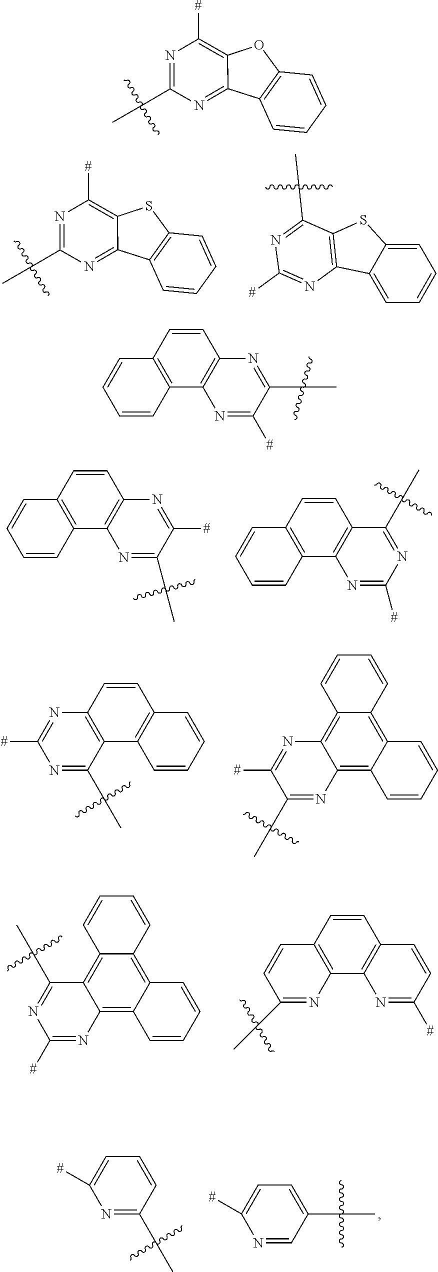

in which: [symbol] represents a bond linked with L; [symbol] represents a bond linked with L₁; [symbol] represents a bond linked with L₂; where a formula contains no [symbol] , it means that, in

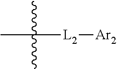

linked at this position, L₂ is a single bond and Ar₂ is hydrogen.

In some embodiments, L, L₁, L₂ and L₃ are identical or different, and are each independently selected from a single bond, a substituted or unsubstituted C₆₋₁₅ arylene, or a substituted or unsubstituted C₅₋₁₈ heteroarylene.

In some embodiments, L, L₁, L₂ and L₃ are identical or different, and are each independently selected from a single bond, a substituted or unsubstituted C₆, C₇, C₈, C₉, C₁₀, C₁₁, C₁₂, C₁₃, C₁₄ or C₁₅ arylene, or a substituted or unsubstituted C₅, C₆, C₇, C₈, C₉, C₁₀, C₁₁, C₁₂, C₁₃, C₁₄, C₁₅, C₁₆, C₁₇ or C₁₈ heteroarylene.

Alternatively, substituents of L, L₁, L₂ and L₃ are each independently selected from deuterium, a fluorine, a cyano, an C₁₋₅, alkyl, a C₃₋₈ trialkylsilyl, a C₁₋₄ fluoroalkyl, a C₁₋₄ deuterated alkyl, a phenyl or a naphthyl.

In some embodiments, L, L₁, L₂ and L₃ are identical or different, and are each independently selected from a single bond, a substituted or unsubstituted phenylene, a substituted or unsubstituted naphthylene, a substituted or unsubstituted biphenylene, a substituted or unsubstituted anthrylene, a substituted or unsubstituted phenanthrylene, a substituted or unsubstituted fluorenylidene, a substituted or unsubstituted pyridylidene, a substituted or unsubstituted dibenzothiophenylene, a substituted or unsubstituted dibenzofuranylene, or a substituted or unsubstituted carbazolylene.

Alternatively, substituents of L, L₁, L₂ and L₃ are identical or different, and are each independently selected from deuterium, a fluorine, a cyano, a methyl, an ethyl, an isopropyl, a tert-butyl, a trifluoromethyl, a trideuteromethyl, a trimethylsilyl or a phenyl.

In some embodiments, L is selected from a single bond or the following groups:

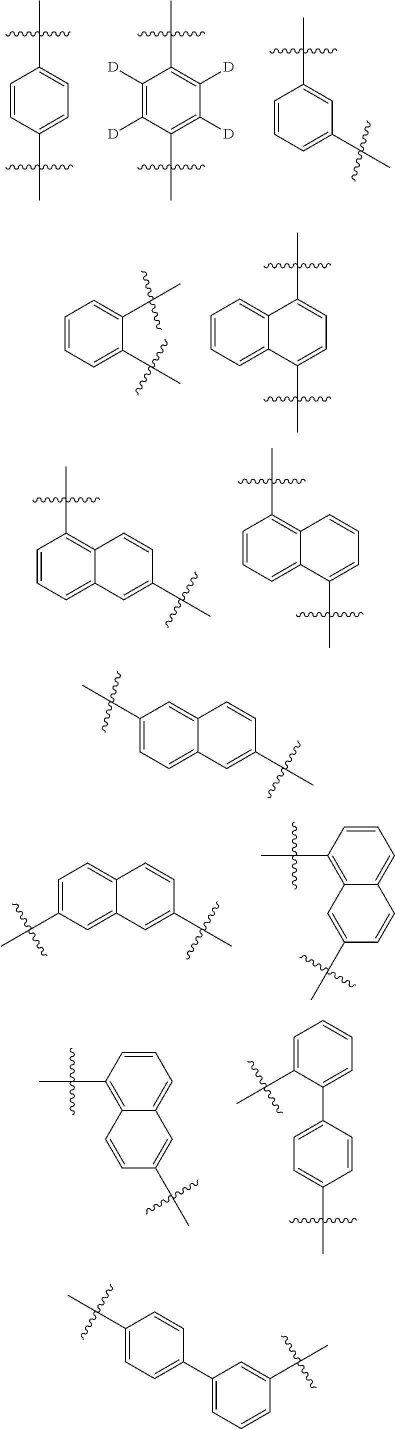

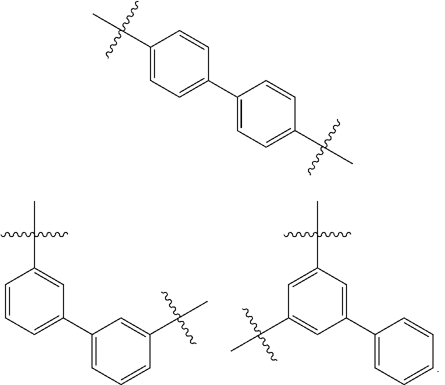

In some embodiments, L₁, L₂ and L₃ are each independently selected from a single bond or the following groups:

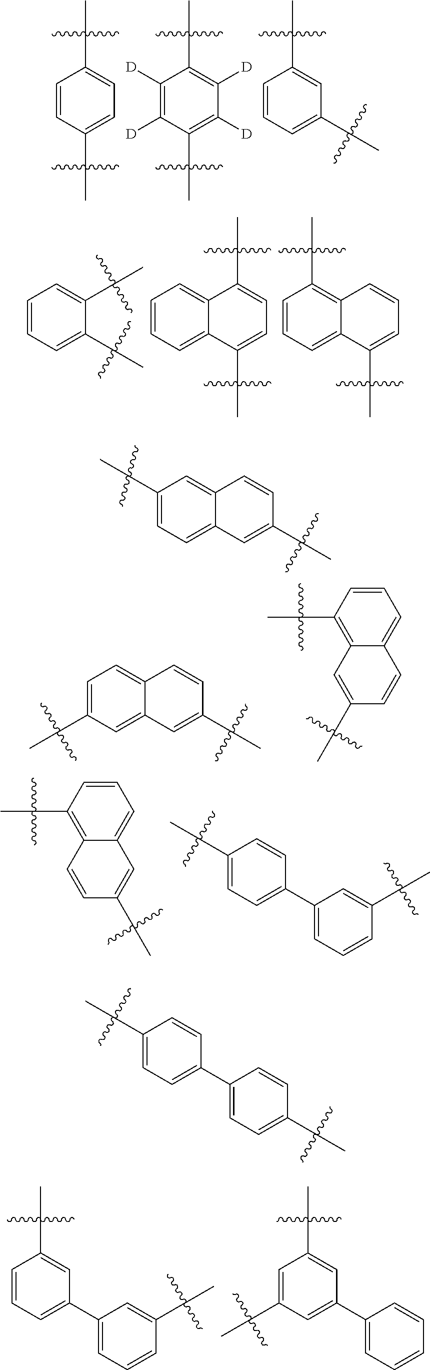

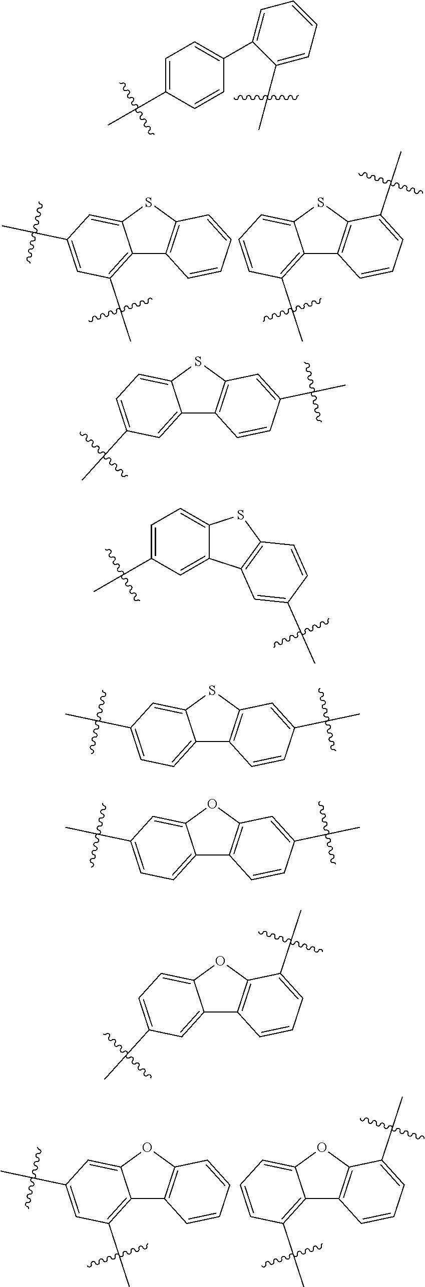

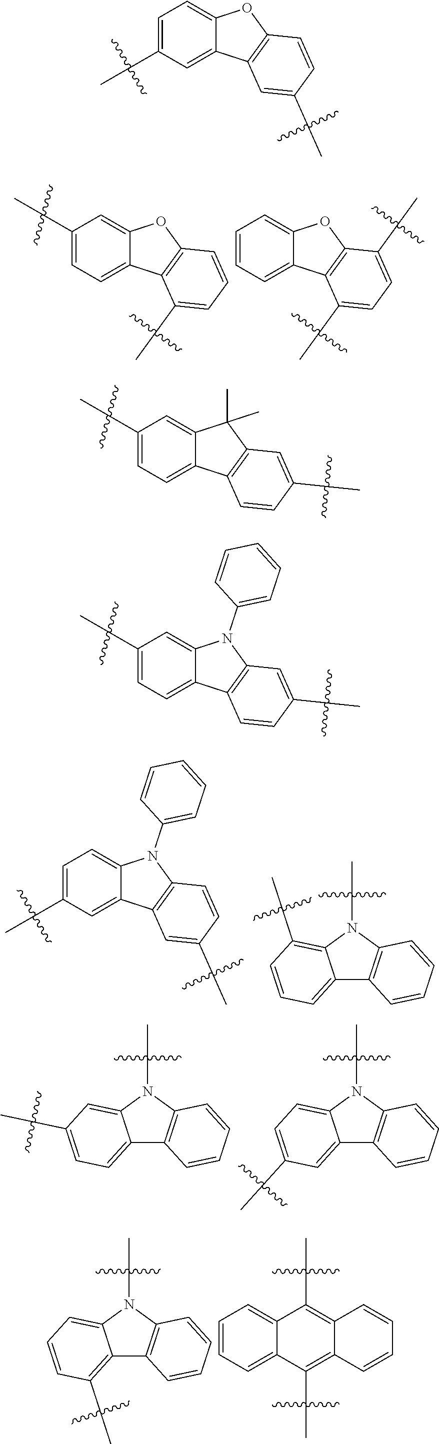

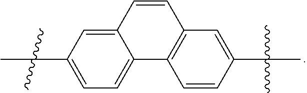 [SMILES: *c1ccc2c(ccc3cc(*)ccc32)c1]

In some embodiments, Ar₁ and Ar₂ are identical or different, and are each independently selected from hydrogen, a substituted or unsubstituted C₆₋₂₅ aryl, or a substituted or unsubstituted C₅₋₂₀ heteroaryl.

In some embodiments, Ar₁ is selected from a substituted or unsubstituted C₆₋₂₅ aryl, or a substituted or unsubstituted C₇₋₂₀ heteroaryl; and Ar₂ is selected from hydrogen, a substituted or unsubstituted C₆₋₂₅ aryl, or a substituted or unsubstituted C₇₋₂₀ heteroaryl.

In some embodiments, substituents of Ar₁ and Ar₂ are each independently selected from deuterium, a halogen, a cyano, a C₁₋₄ haloalkyl, a C₁₋₄ deuterated alkyl, an C₁₋₄ alkyl, a C₅₋₁₀ cycloalkyl, an C₆₋₁₂ aryl, a C₅₋₁₂ heteroaryl, or a C₃₋₈ trialkylsilyl; and optionally, any two adjacent substituents form a benzene ring or a fluorene ring.

In some embodiments, Ar₁ and Ar₂ are each independently selected from hydrogen, a substituted or unsubstituted C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄ or C₂₅ aryl, or a substituted or unsubstituted C₅, C₆, C₇, C₈, C₉, C₁₀, C₁₂, C₁₃, C₁₄, C₁₅, C₁₆, C₁₇, C₁₈, C₁₉ or C₂₀ heteroaryl.

In some embodiments, Ar₃ is selected from a substituted or unsubstituted C₆₋₂₅ aryl, or a substituted or unsubstituted C₇₋₂₀ heteroaryl.

In some embodiments, Ar₃ is selected from selected from a substituted or unsubstituted C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄ or C₂₅ aryl, or a substituted or unsubstituted C₇, C₈, C₉, C₁₀, C₁₂, C₁₃, C₁₄, C₁₅, C₁₆, C₁₇, C₁₈, C₁₉ or C₂₀ heteroaryl.

In some embodiments, substituent of Ar₃ is each independently selected from deuterium, a halogen, a cyano, a C₁₋₄ haloalkyl, a C₁₋₄ deuterated alkyl, an C₁₋₄ alkyl, a C₅₋₁₀ cycloalkyl, an C₆₋₁₂ aryl, a C₅₋₁₂ heteroaryl or a C₃₋₈ trialkylsilyl; and optionally, any two adjacent substituents form a benzene ring or a fluorene ring.

In some embodiments, Ar₁ and Ar₃ are each independently selected from substituted or unsubstituted groups V, and Ar₂ is selected from hydrogen, substituted or unsubstituted groups V. The unsubstituted groups V are selected from the following groups:

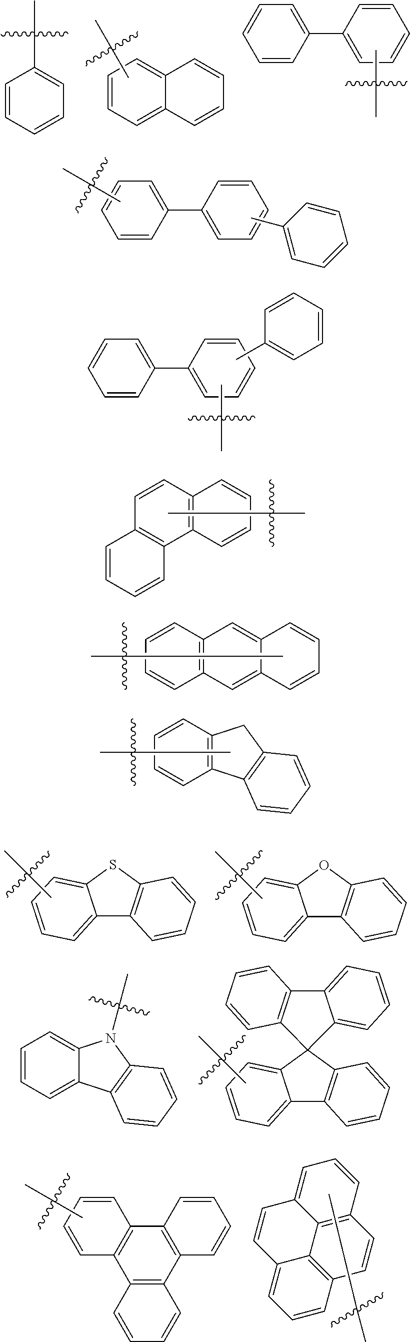

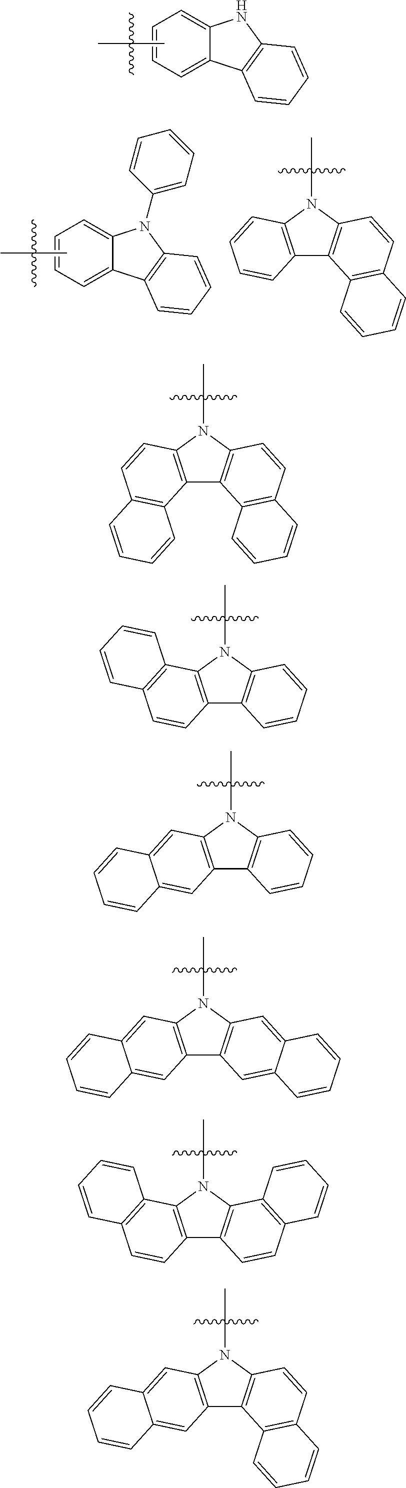

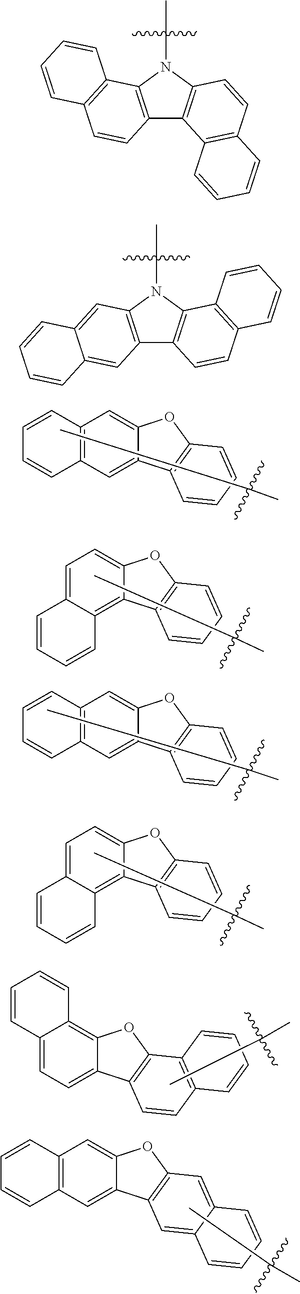

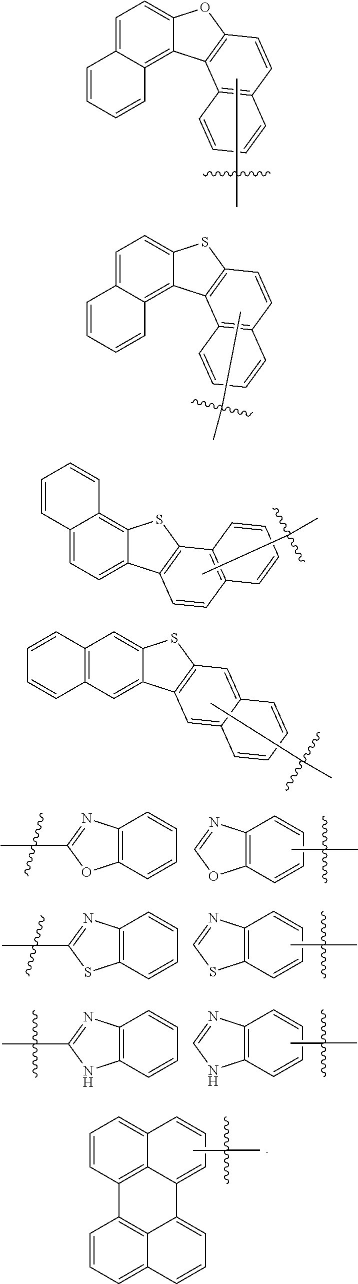

the substituted groups V each have one or more substituents, the substituents being each independently selected from deuterium, a fluorine, a cyano, a trideuteromethyl, a trimethylsilyl, a trifluoromethyl, a cyclopentyl, a cyclohexyl, an adamantly, a methyl, an ethyl, an isopropyl, a tert-butyl, a phenyl, a naphthyl, a pyridyl, a dibenzofuranyl, a dibenzothiophenyl, a carbazolyl, a benzoxazolyl, or a dibenzothiophenyl, and when each group V has more than one substituents, the substituents are identical or different.

In some embodiments, Ar₁ and Ar₃ are each independently selected from the following groups, and Ar₂ is selected from hydrogen and the following groups:

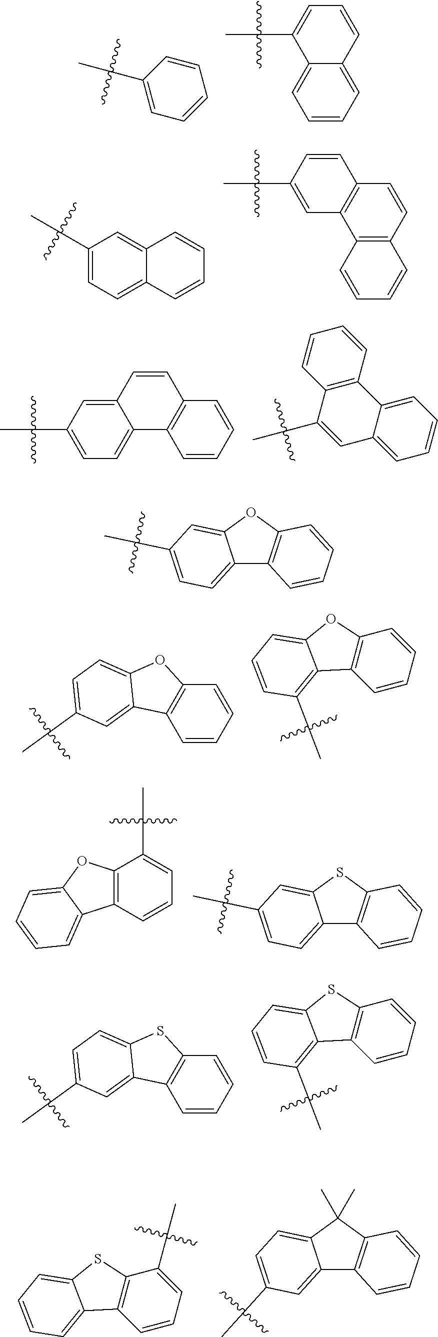

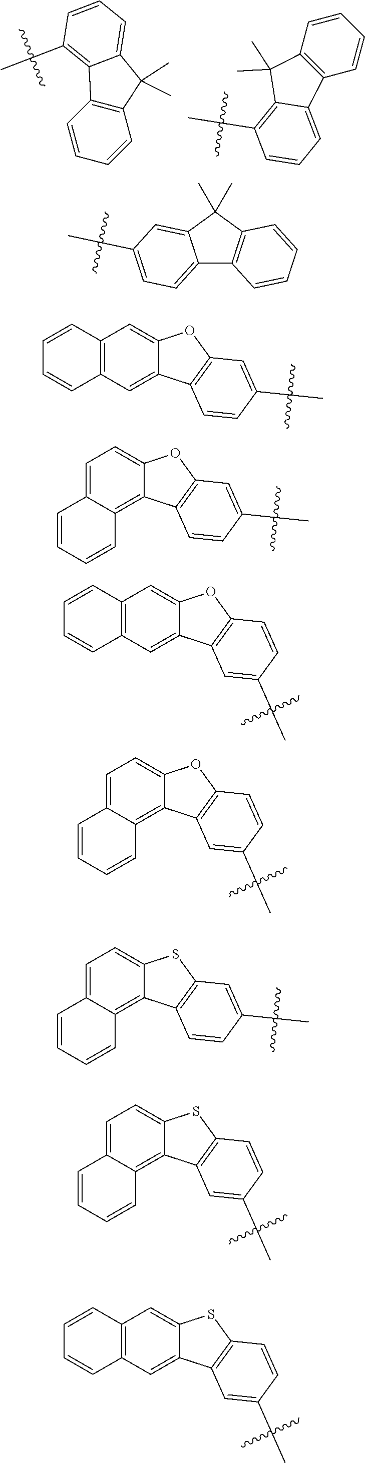

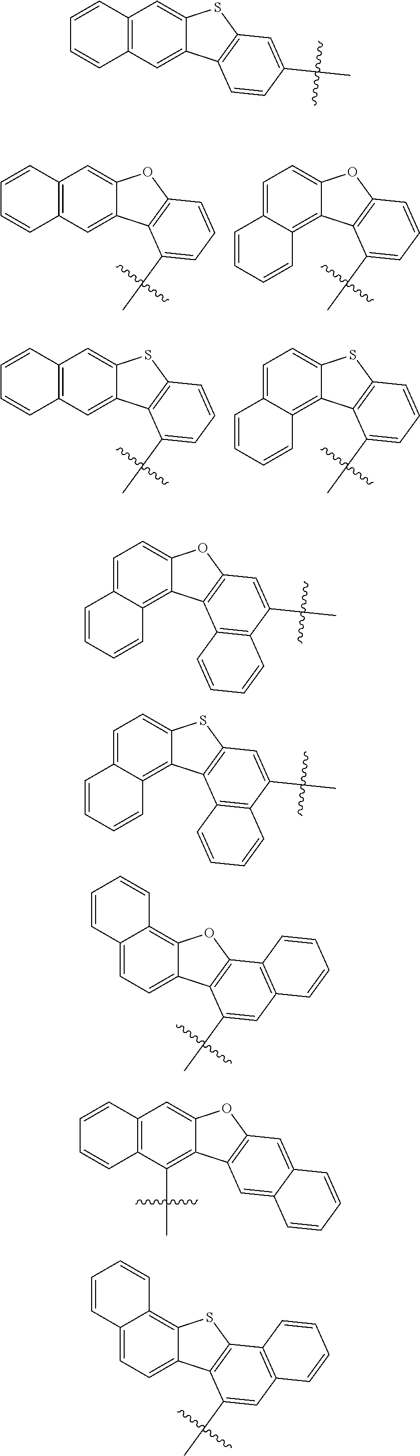

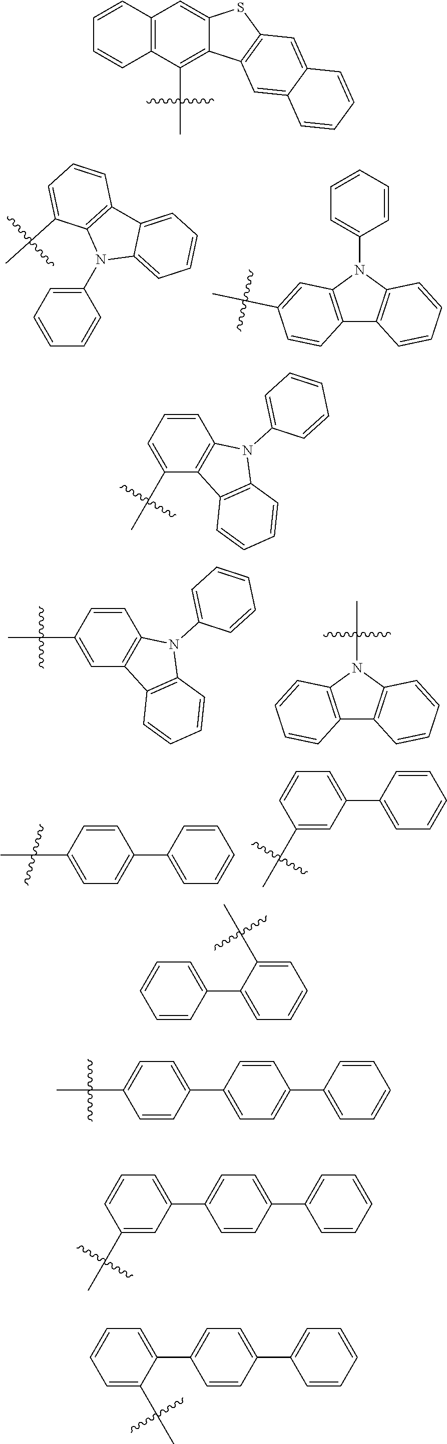

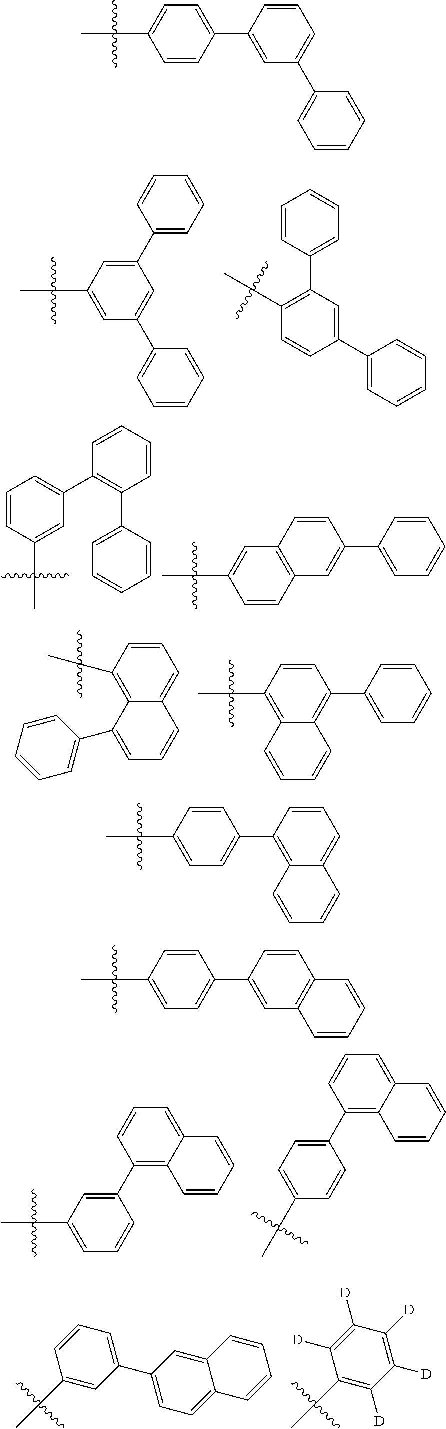

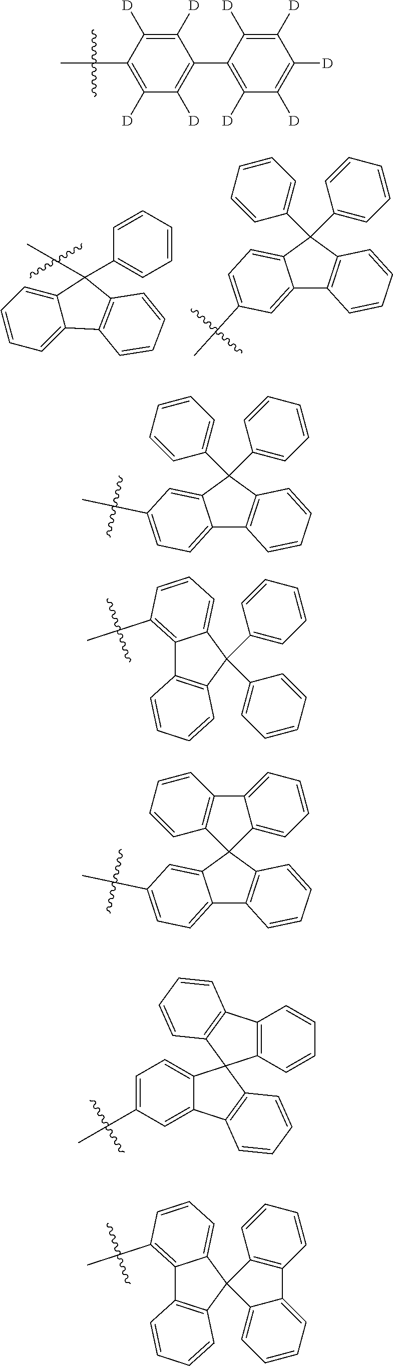

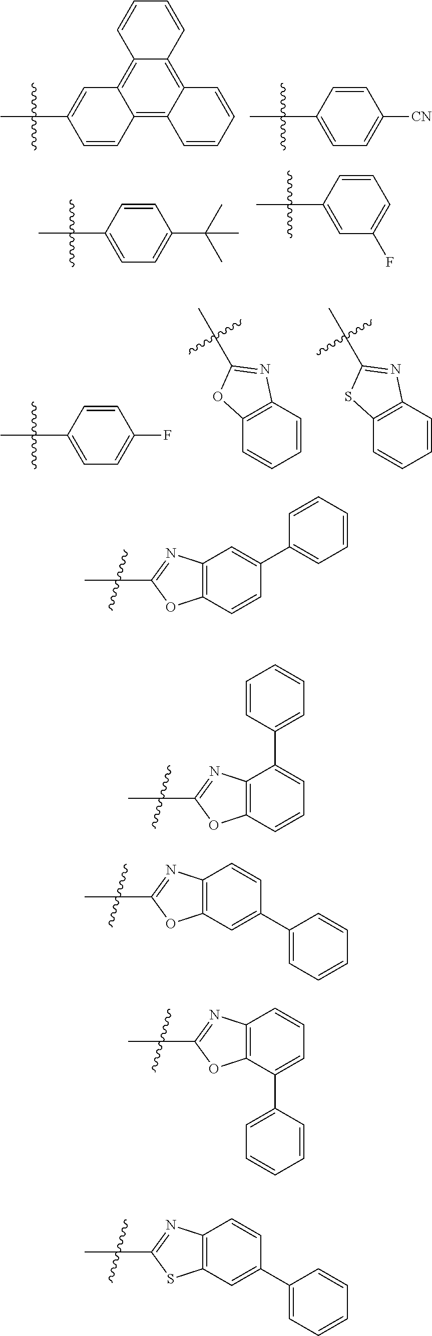

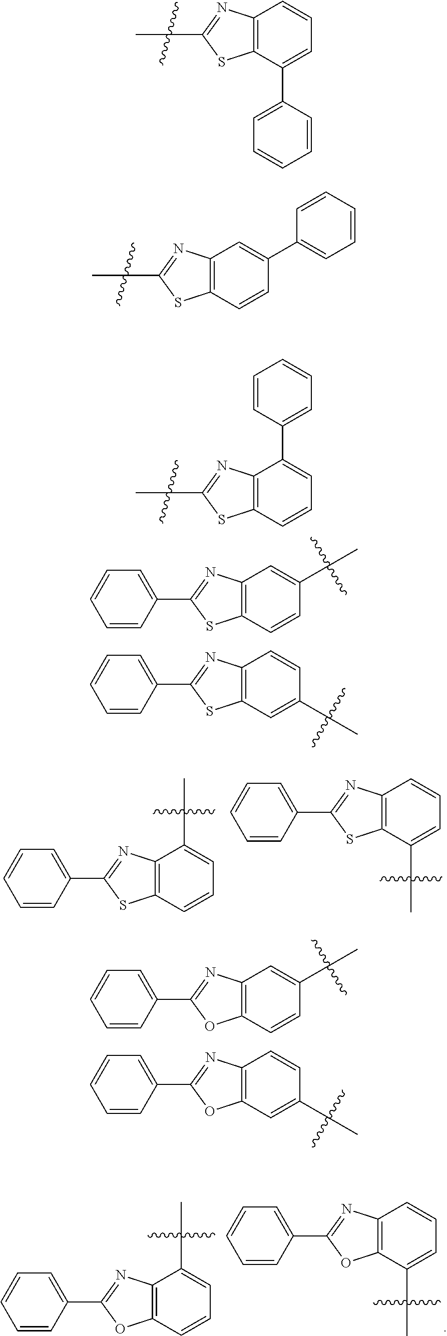

In some embodiments, at least one of A₁ and A₂ is the structure shown in Formula a-1, and the structure

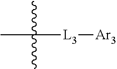

shown in Formula a-1 is selected from the following groups:

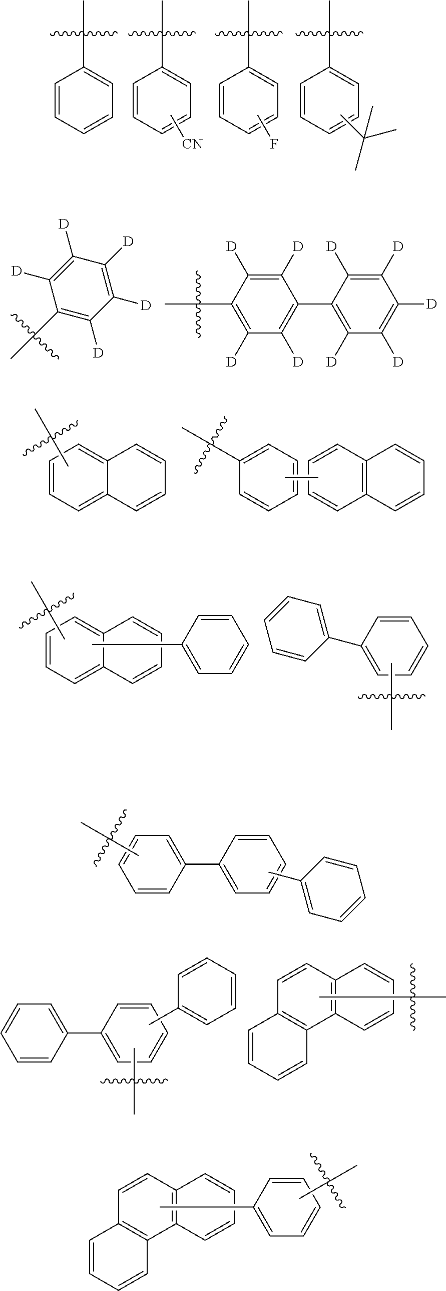

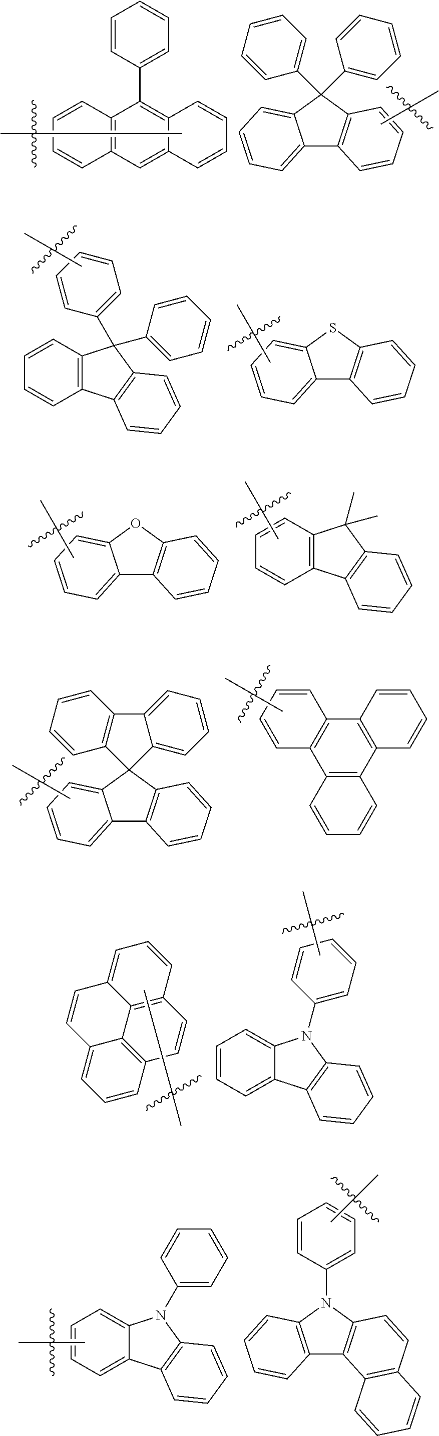

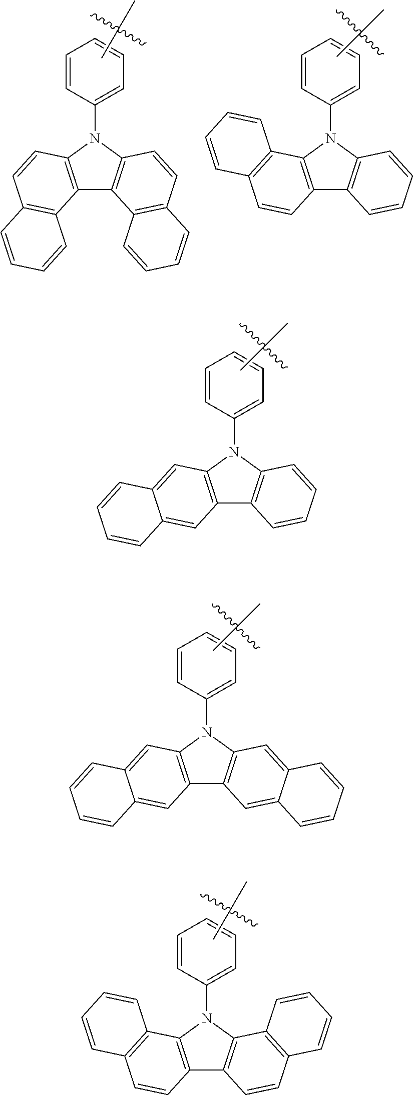

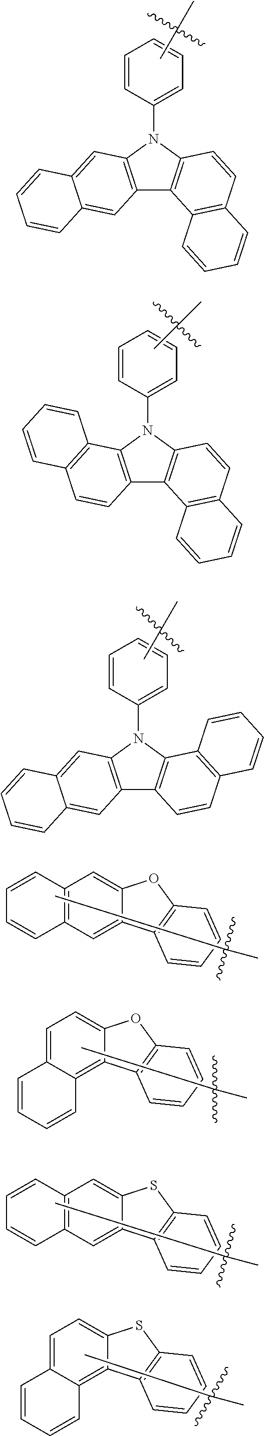

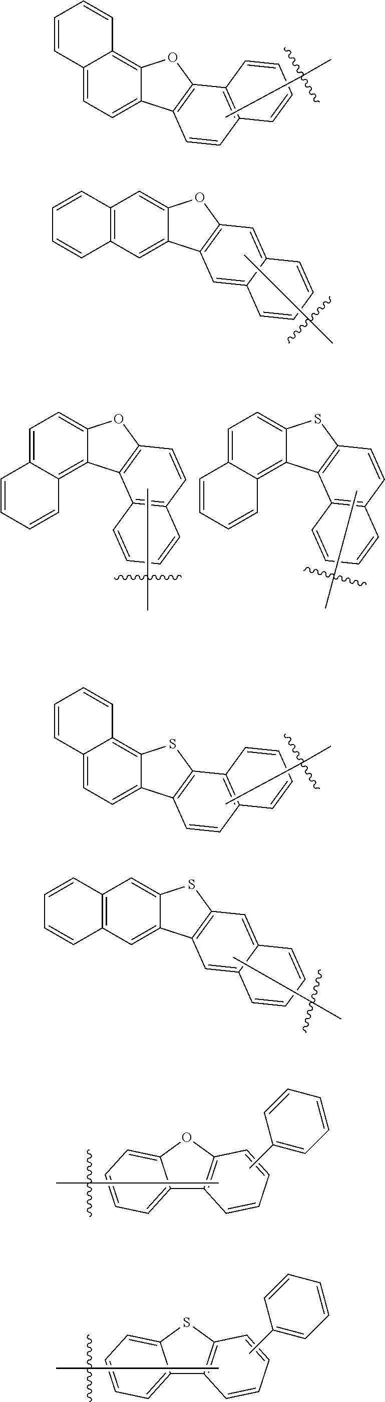

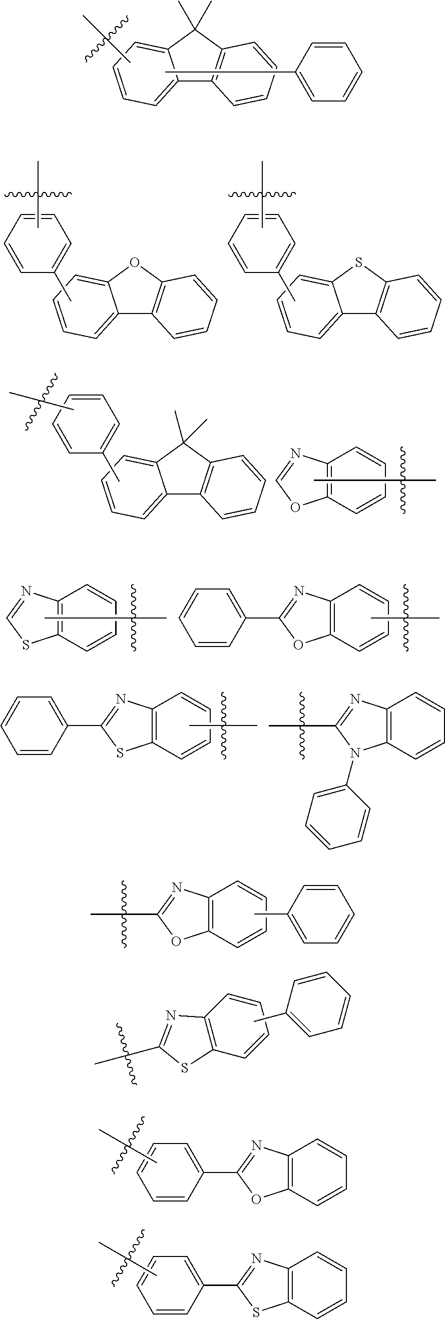

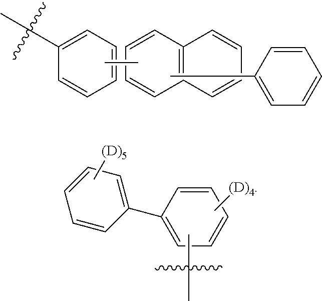

In some embodiments, the structure

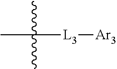

shown in Formula a-1 is selected from the group consisting of the following groups:

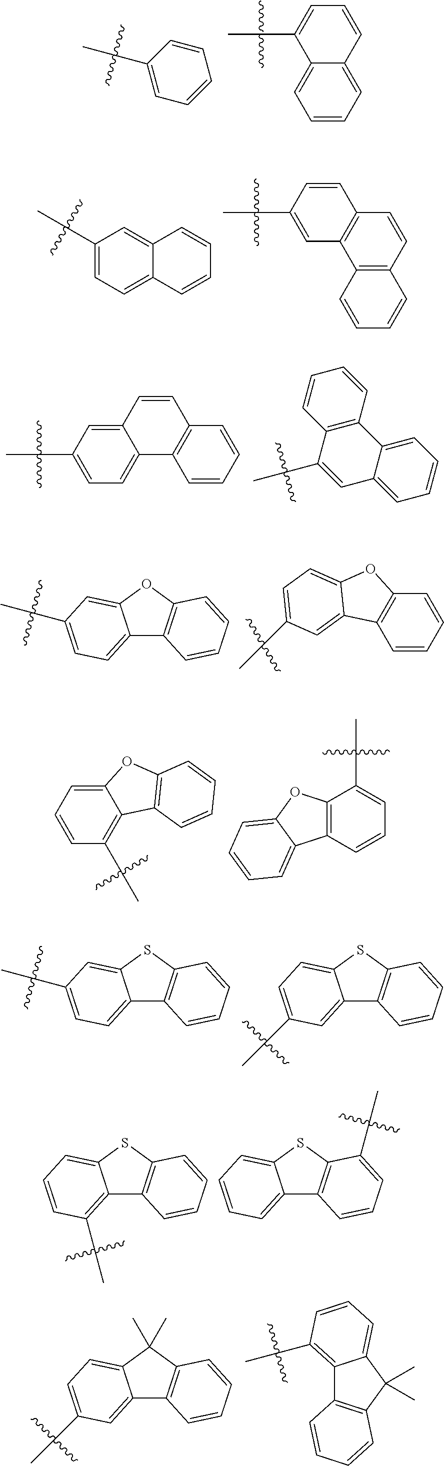

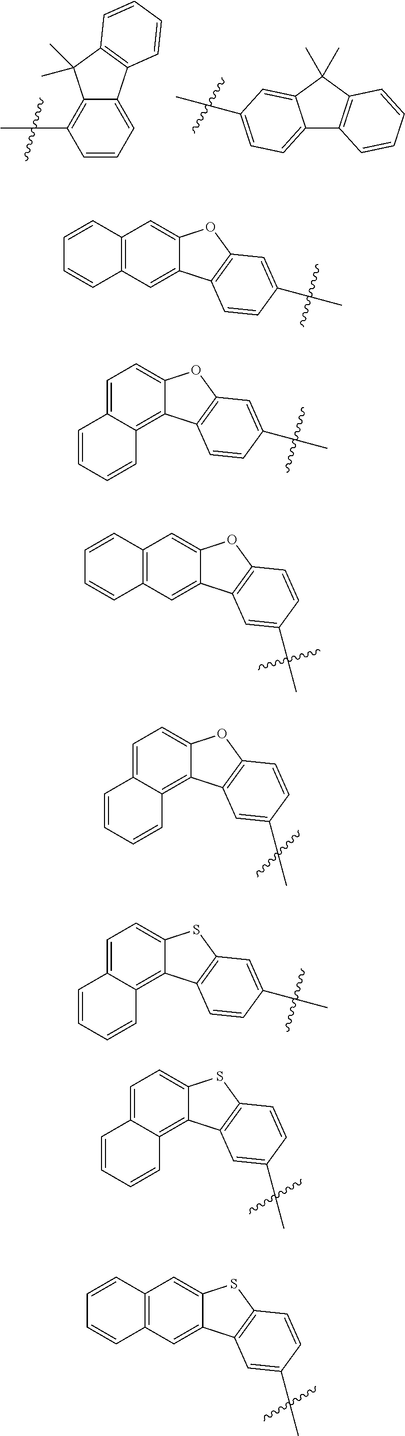

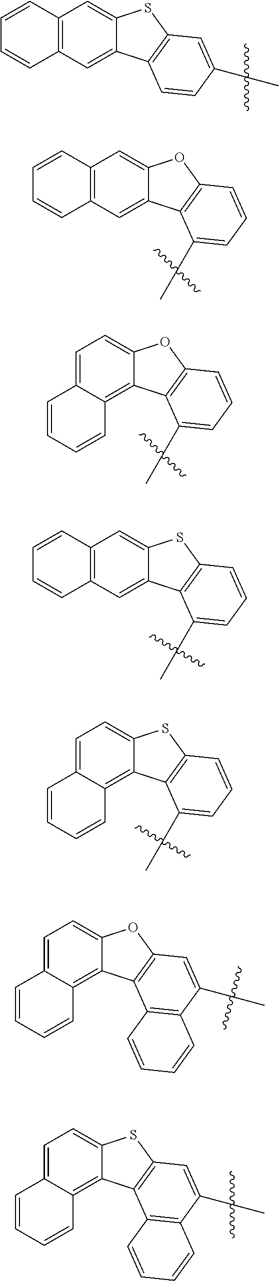

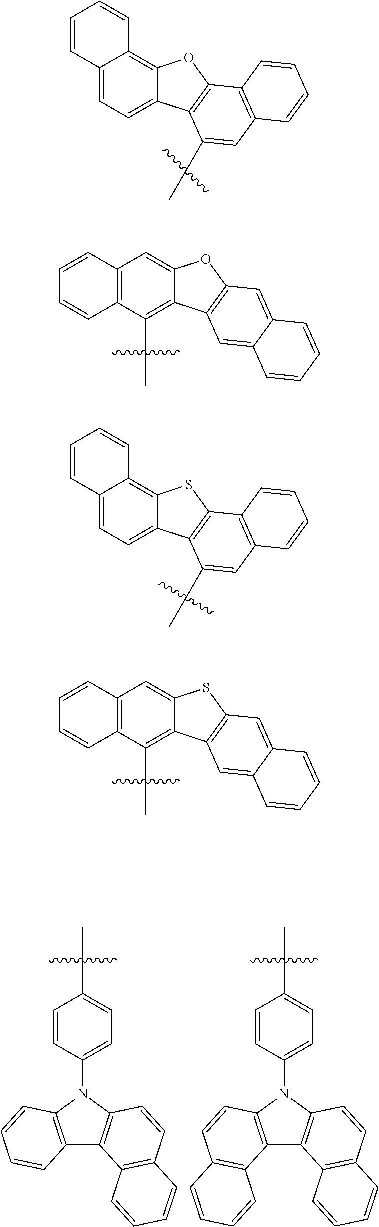

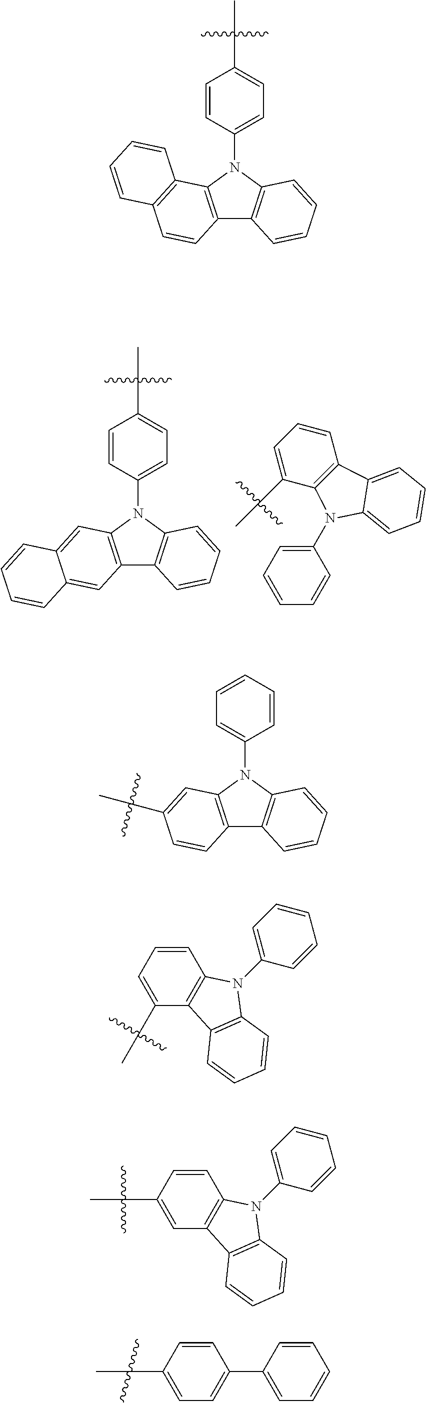

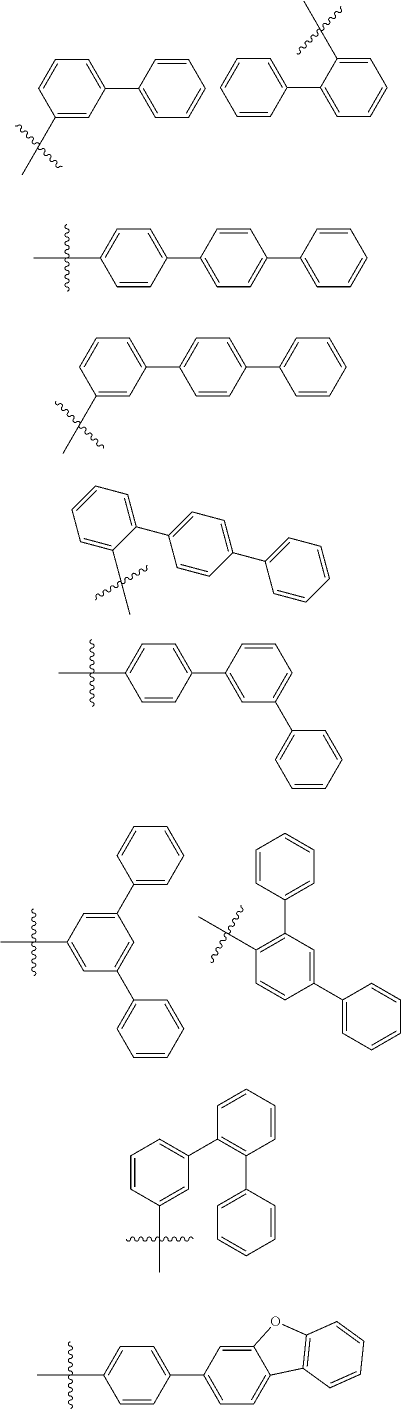

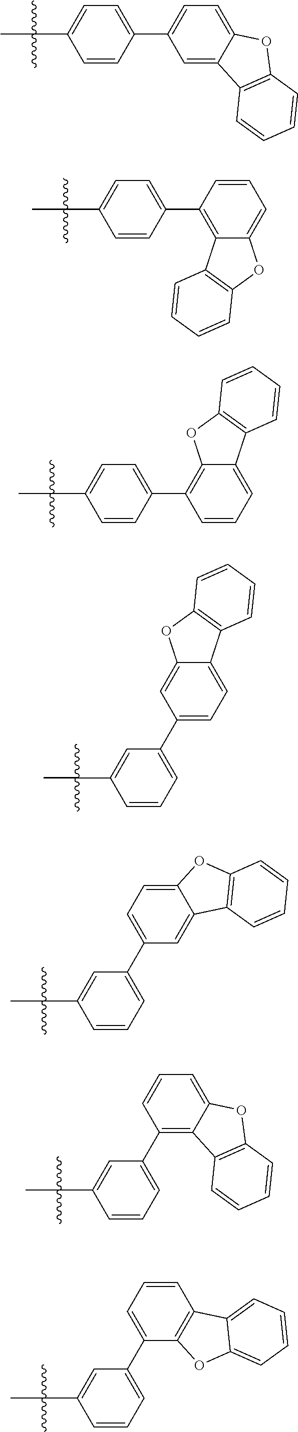

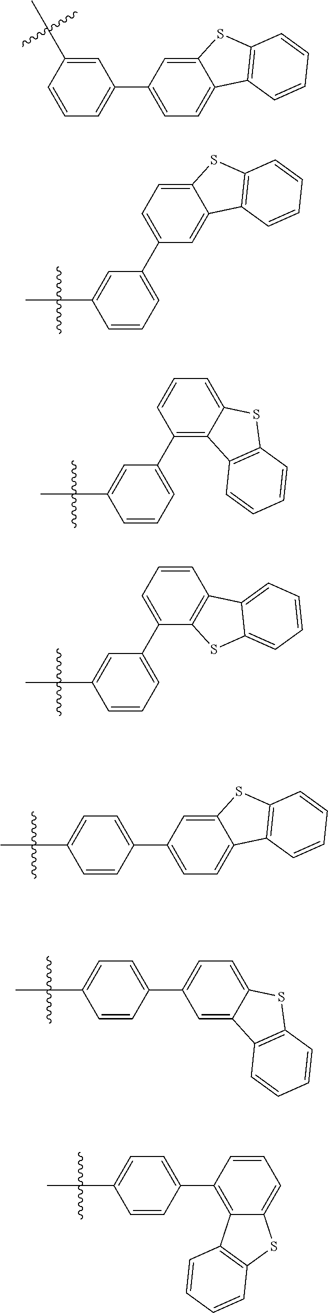

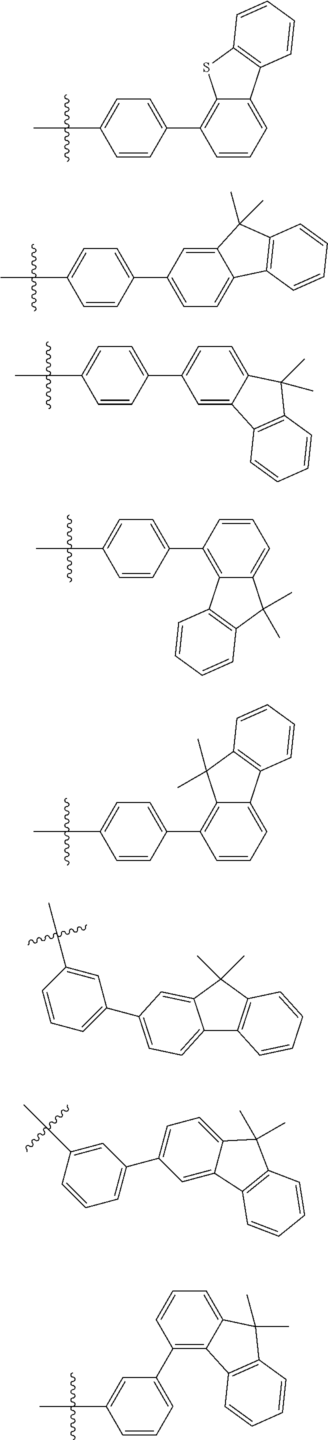

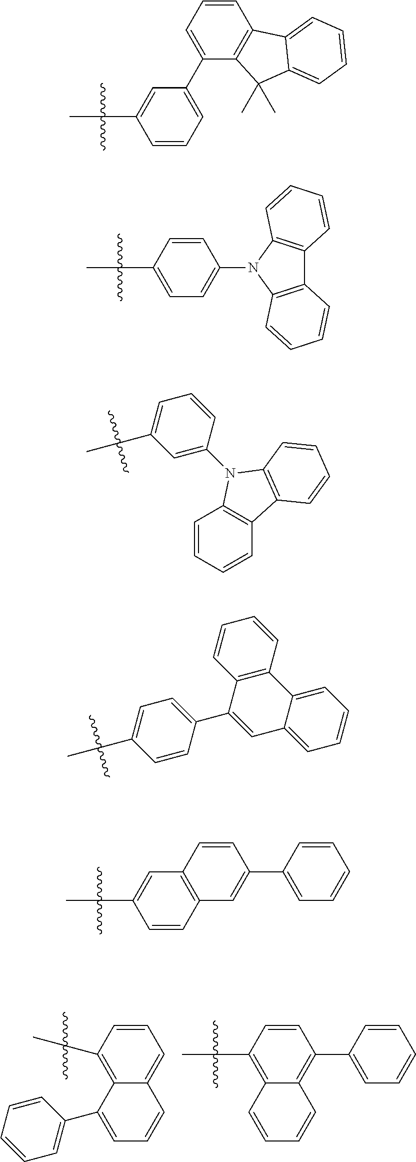

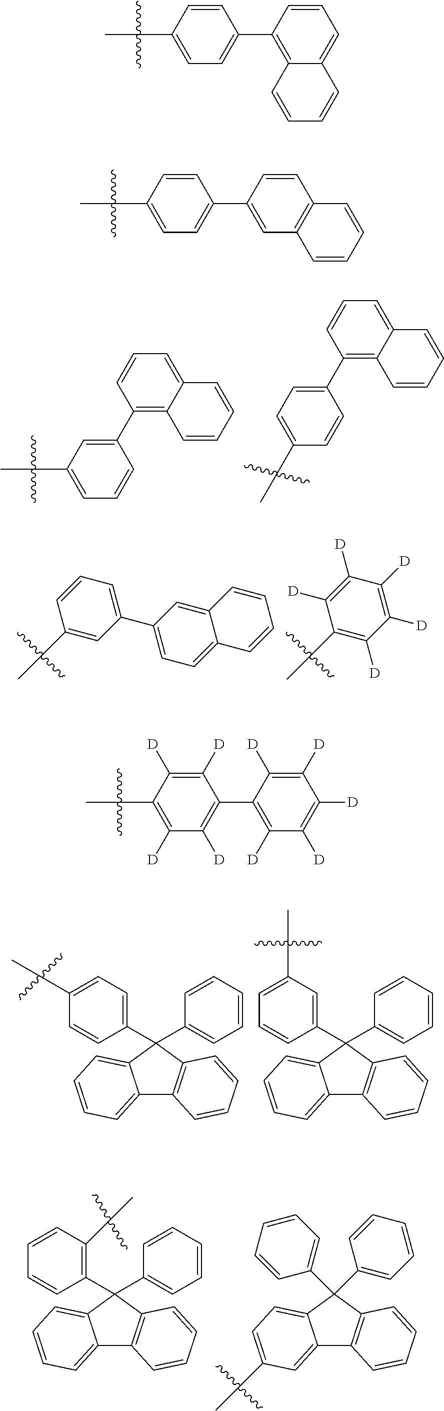

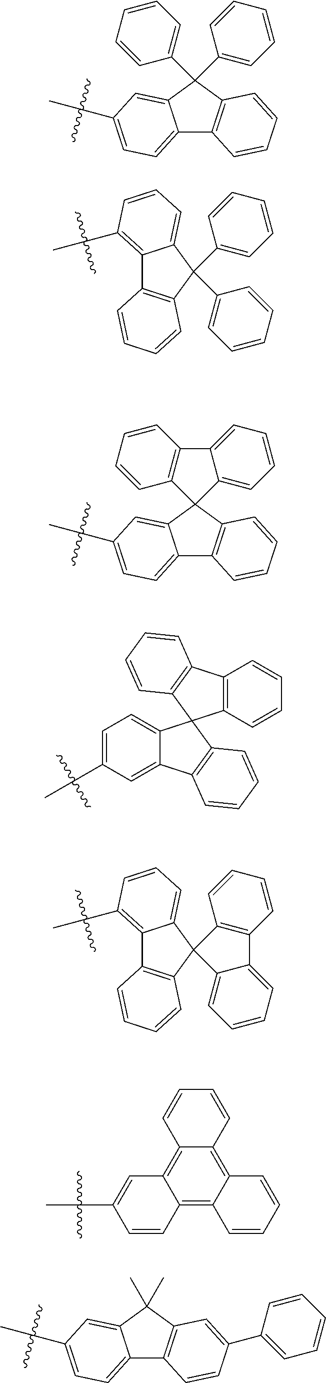

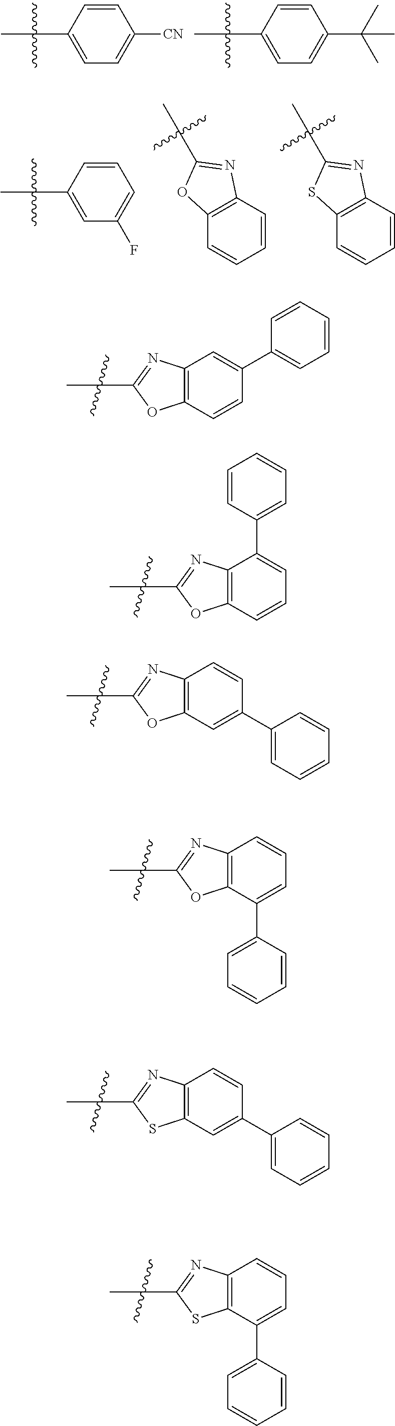

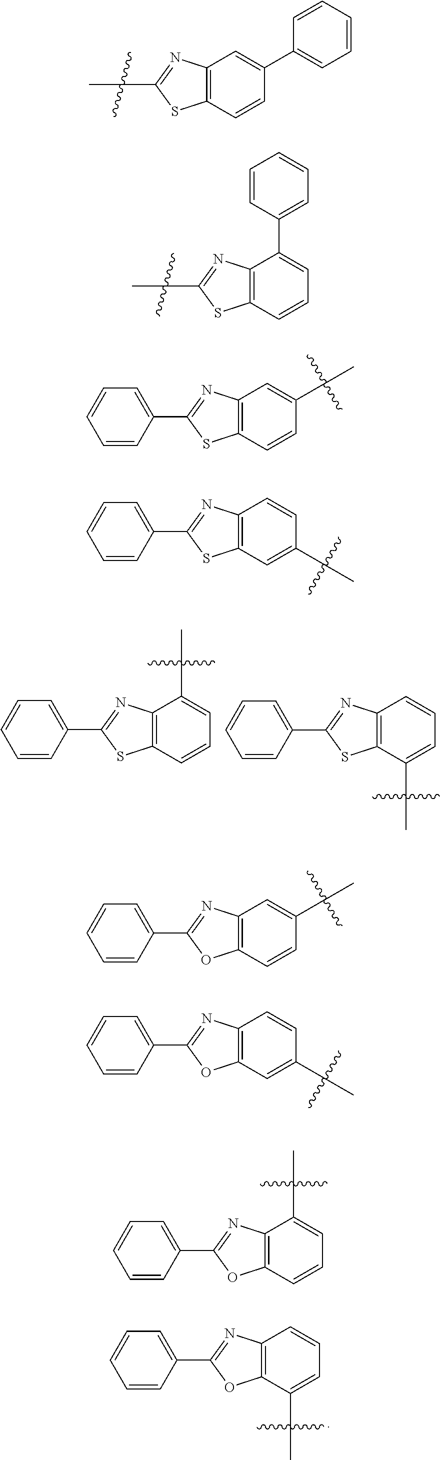

In some embodiments,

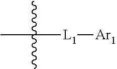

is selected from the group consisting of the following groups, and

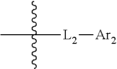

is selected from hydrogen or the following groups:

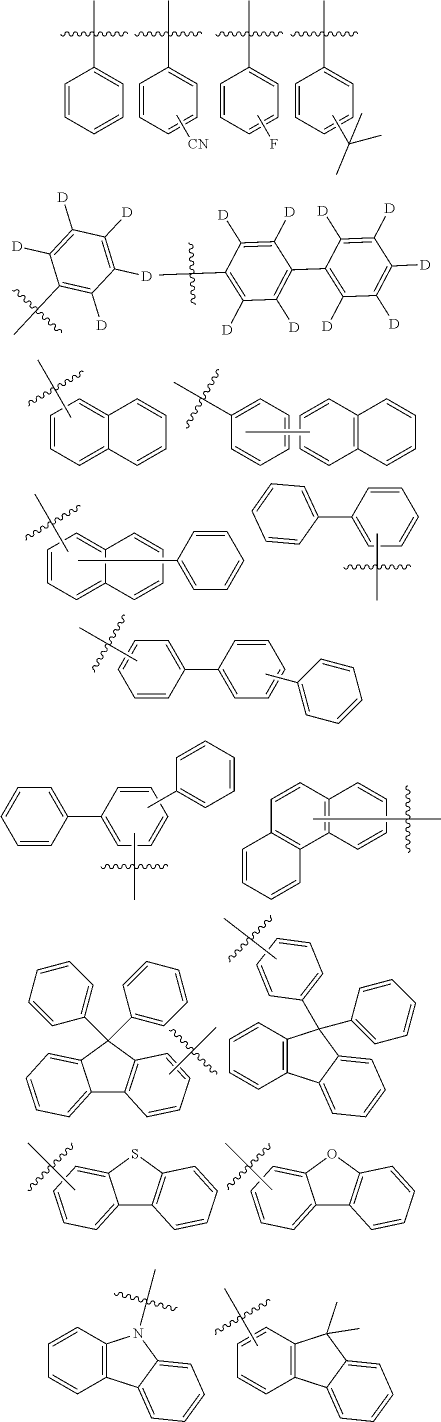

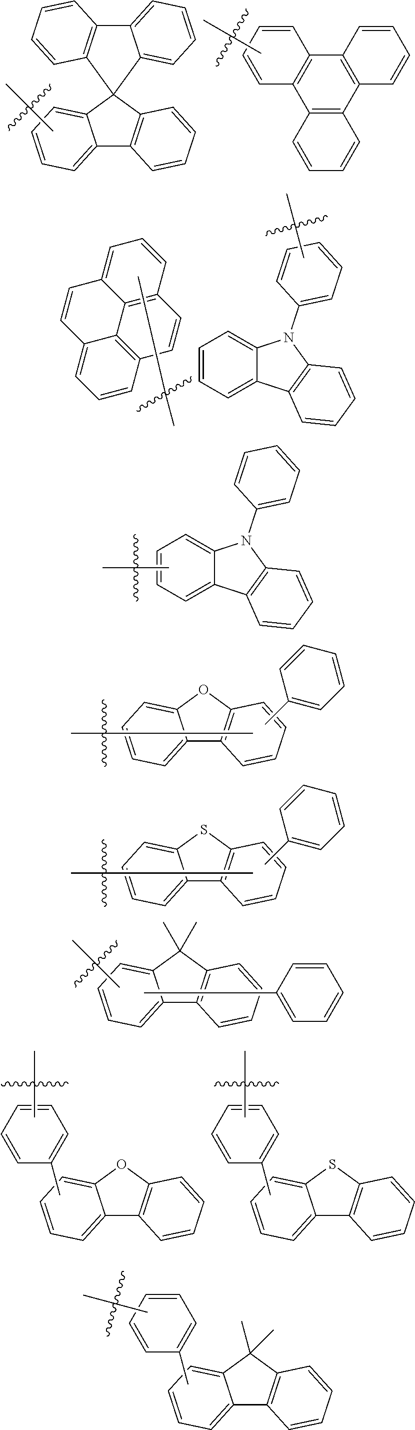

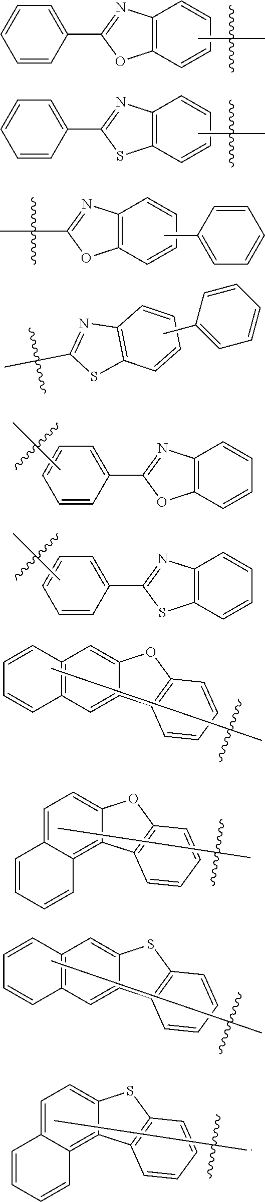

In some embodiments, the structure

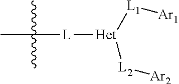

shown in Formula a-2 is selected from the group consisting of the following groups:

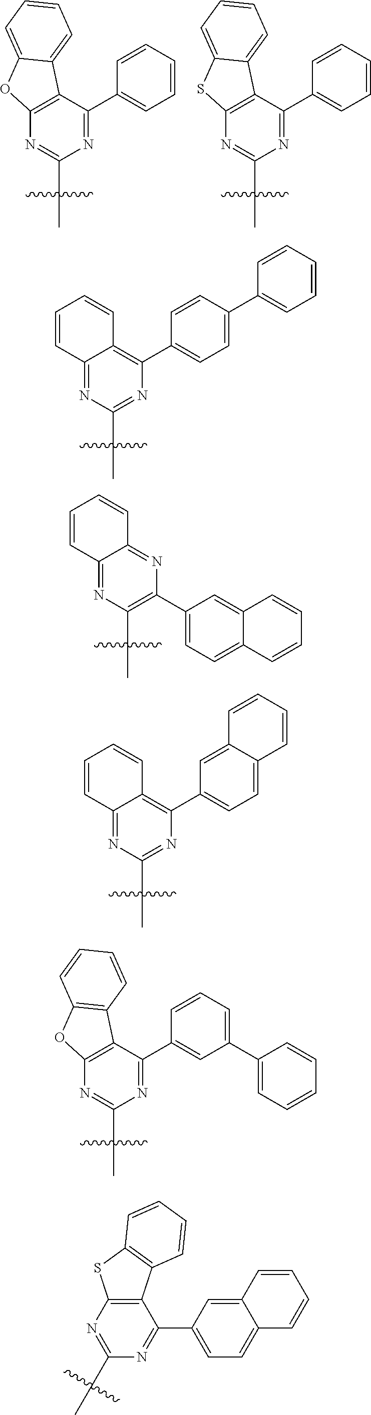

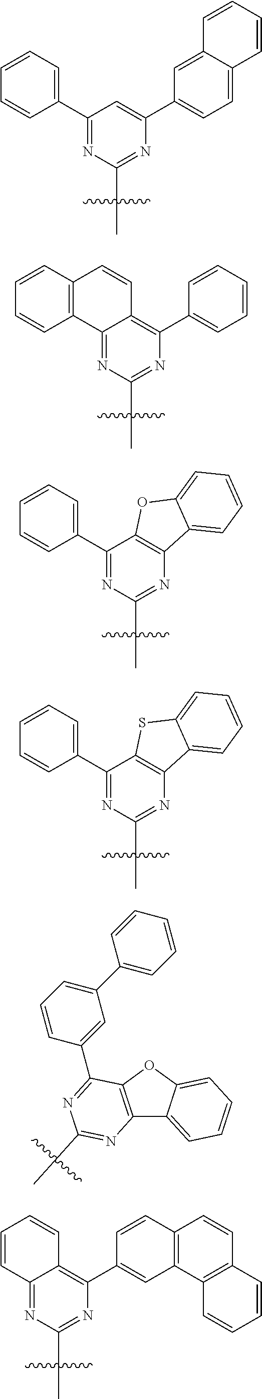 [SMILES: *c1nc(-c2ccccc2)cc(-c2ccc3ccccc3c2)n1 ; *c1nc(-c2ccccc2)c2ccc3ccccc3c2n1 ; *c1nc(-c2ccccc2)c2oc3ccccc3c2n1 ; *c1nc(-c2ccccc2)c2sc3ccccc3c2n1 ; *c1nc(-c2cccc(-c3ccccc3)c2)c2oc3ccccc3c2n1 ; *c1nc(-c2ccc3ccc4ccccc4c3c2)c2ccccc2n1]

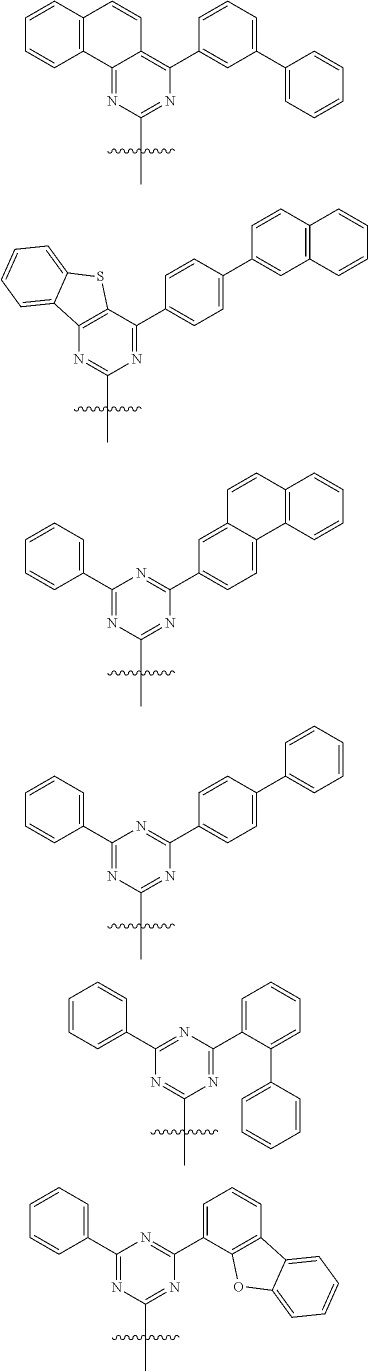 [SMILES: *c1nc(-c2cccc(-c3ccccc3)c2)c2ccc3ccccc3c2n1 ; *c1nc(-c2ccc(-c3ccc4ccccc4c3)cc2)c2sc3ccccc3c2n1 ; *c1nc(-c2ccccc2)nc(-c2ccc3c(ccc4ccccc43)c2)n1 ; *c1nc(-c2ccccc2)nc(-c2ccc(-c3ccccc3)cc2)n1 ; *c1nc(-c2ccccc2)nc(-c2ccccc2-c2ccccc2)n1 ; *c1nc(-c2ccccc2)nc(-c2cccc3c2oc2ccccc23)n1]

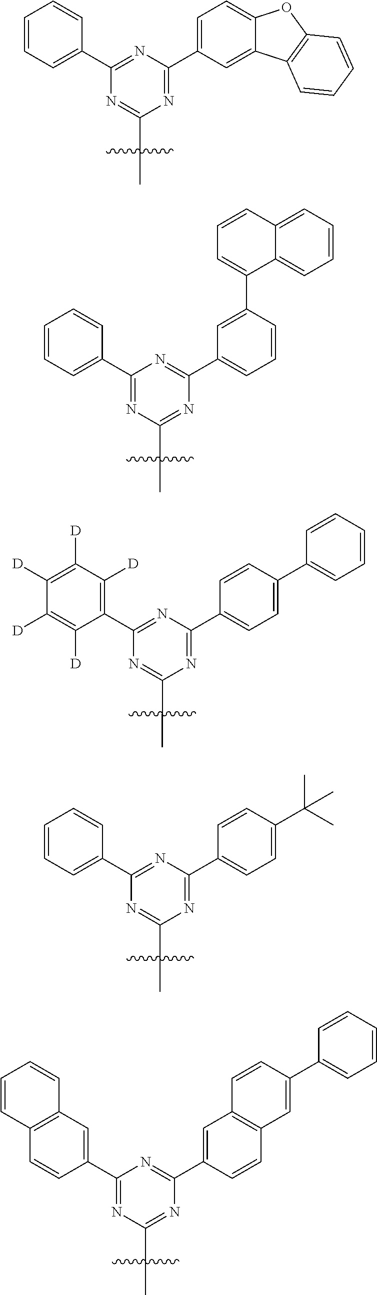 [SMILES: *c1nc(-c2ccccc2)nc(-c2ccc3oc4ccccc4c3c2)n1 ; *c1nc(-c2ccccc2)nc(-c2cccc(-c3cccc4ccccc34)c2)n1 ; *c1nc(-c2ccc(-c3ccccc3)cc2)nc(-c2c([2H])c([2H])c([2H])c([2H])c2[2H])n1 ; *c1nc(-c2ccccc2)nc(-c2ccc(C(C)(C)C)cc2)n1 ; *c1nc(-c2ccc3ccccc3c2)nc(-c2ccc3cc(-c4ccccc4)ccc3c2)n1]

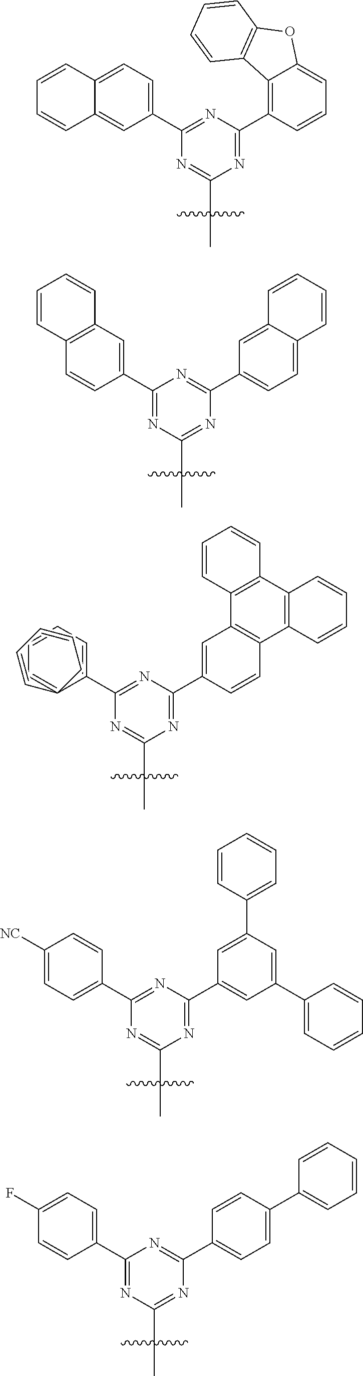

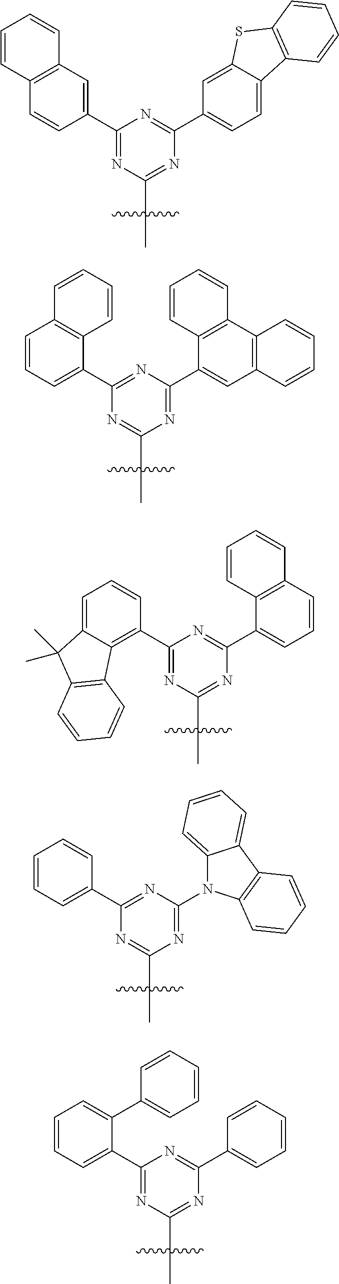 [SMILES: *c1nc(-c2ccc3ccccc3c2)nc(-c2ccc3c(c2)sc2ccccc23)n1 ; *c1nc(-c2cccc3ccccc23)nc(-c2cc3ccccc3c3ccccc23)n1 ; *c1nc(-c2cccc3c2-c2ccccc2C3(C)C)nc(-c2cccc3ccccc23)n1 ; *c1nc(-c2ccccc2)nc(-n2c3ccccc3c3ccccc32)n1 ; *c1nc(-c2ccccc2)nc(-c2ccccc2-c2ccccc2)n1]

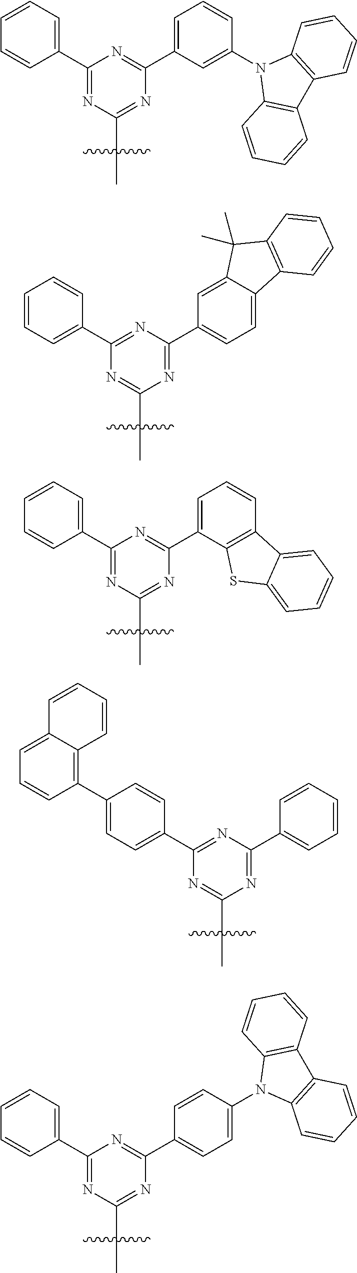 [SMILES: *c1nc(-c2ccccc2)nc(-c2cccc(-n3c4ccccc4c4ccccc43)c2)n1 ; *c1nc(-c2ccccc2)nc(-c2ccc3c(c2)C(C)(C)c2ccccc2-3)n1 ; *c1nc(-c2ccccc2)nc(-c2cccc3c2sc2ccccc23)n1 ; *c1nc(-c2ccccc2)nc(-c2ccc(-c3cccc4ccccc34)cc2)n1 ; *c1nc(-c2ccccc2)nc(-c2ccc(-n3c4ccccc4c4ccccc43)cc2)n1]

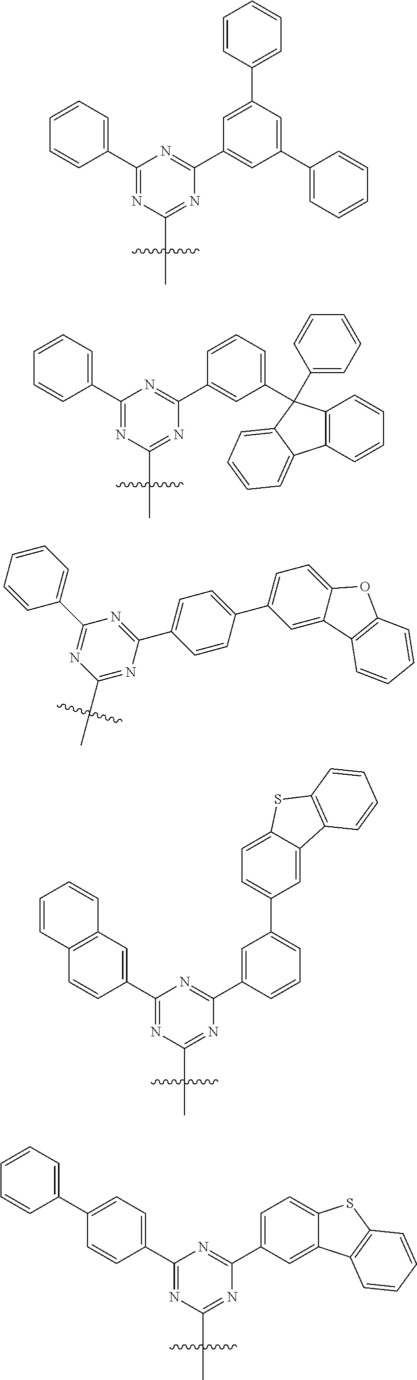 [SMILES: *c1nc(-c2ccccc2)nc(-c2cc(-c3ccccc3)cc(-c3ccccc3)c2)n1 ; *c1nc(-c2ccccc2)nc(-c2cccc(C3(c4ccccc4)c4ccccc4-c4ccccc43)c2)n1 ; *c1nc(-c2ccccc2)nc(-c2ccc(-c3ccc4oc5ccccc5c4c3)cc2)n1 ; *c1nc(-c2cccc(-c3ccc4sc5ccccc5c4c3)c2)nc(-c2ccc3ccccc3c2)n1 ; *c1nc(-c2ccc(-c3ccccc3)cc2)nc(-c2ccc3sc4ccccc4c3c2)n1]

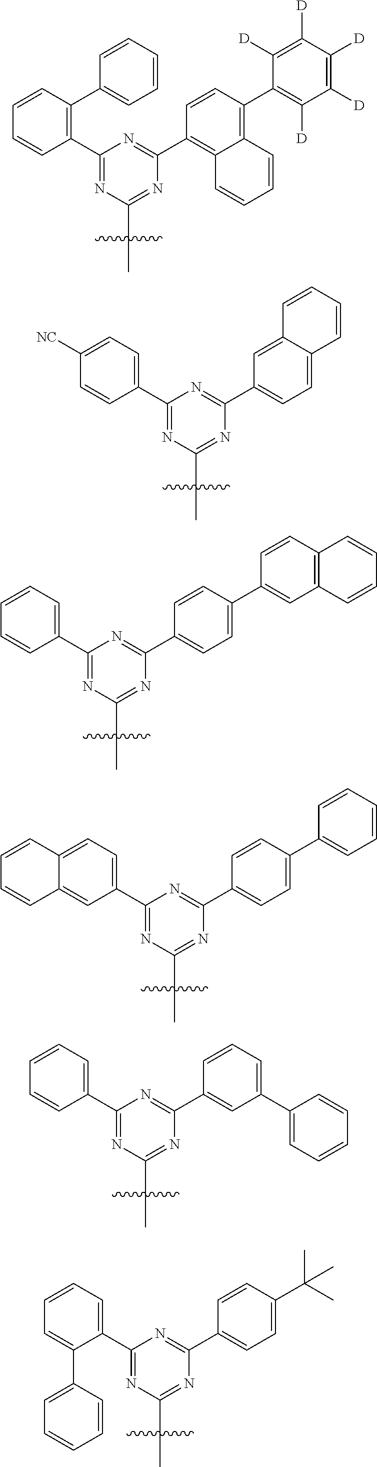 [SMILES: *c1nc(-c2ccccc2-c2ccccc2)nc(-c2ccc(-c3c([2H])c([2H])c([2H])c([2H])c3[2H])c3ccccc23)n1 ; *c1nc(-c2ccc(C#N)cc2)nc(-c2ccc3ccccc3c2)n1 ; *c1nc(-c2ccccc2)nc(-c2ccc(-c3ccc4ccccc4c3)cc2)n1 ; *c1nc(-c2ccc(-c3ccccc3)cc2)nc(-c2ccc3ccccc3c2)n1 ; *c1nc(-c2ccccc2)nc(-c2cccc(-c3ccccc3)c2)n1 ; *c1nc(-c2ccc(C(C)(C)C)cc2)nc(-c2ccccc2-c2ccccc2)n1]

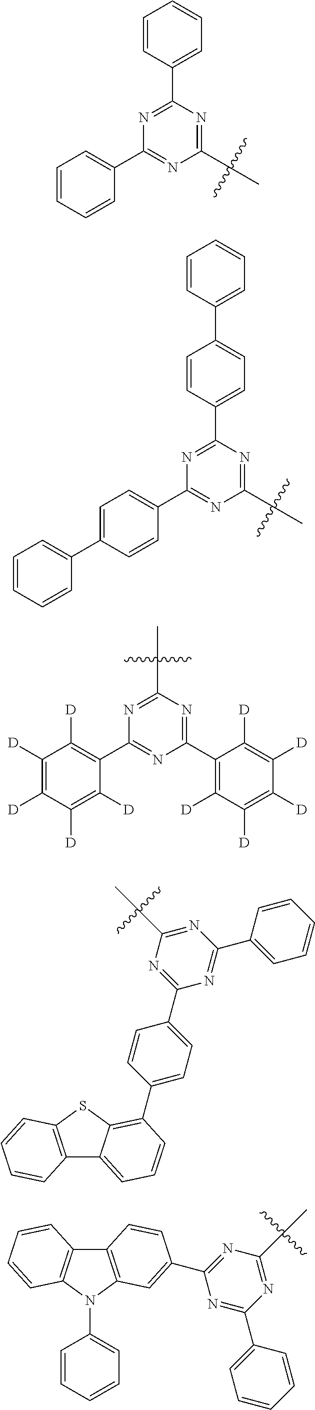

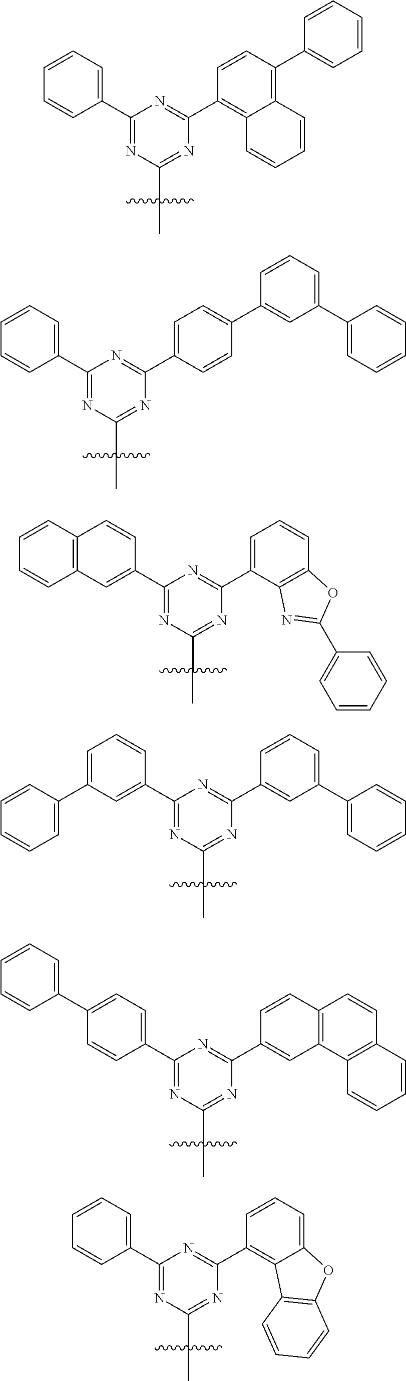

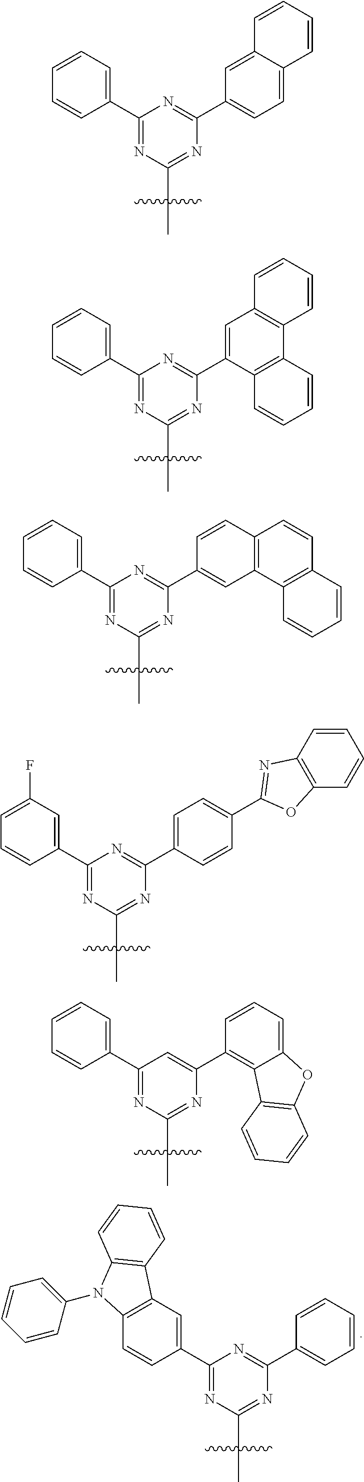 [SMILES: *c1nc(-c2ccccc2)nc(-c2ccc3ccccc3c2)n1 ; *c1nc(-c2ccccc2)nc(-c2cc3ccccc3c3ccccc23)n1 ; *c1nc(-c2ccccc2)nc(-c2ccc3ccc4ccccc4c3c2)n1 ; *c1nc(-c2ccc(-c3nc4ccccc4o3)cc2)nc(-c2cccc(F)c2)n1 ; *c1nc(-c2ccccc2)cc(-c2cccc3oc4ccccc4c23)n1 ; *c1nc(-c2ccccc2)nc(-c2ccc3c(c2)c2ccccc2n3-c2ccccc2)n1]

Alternatively, the nitrogen-containing compound is selected from the group consisting of compounds shown below:

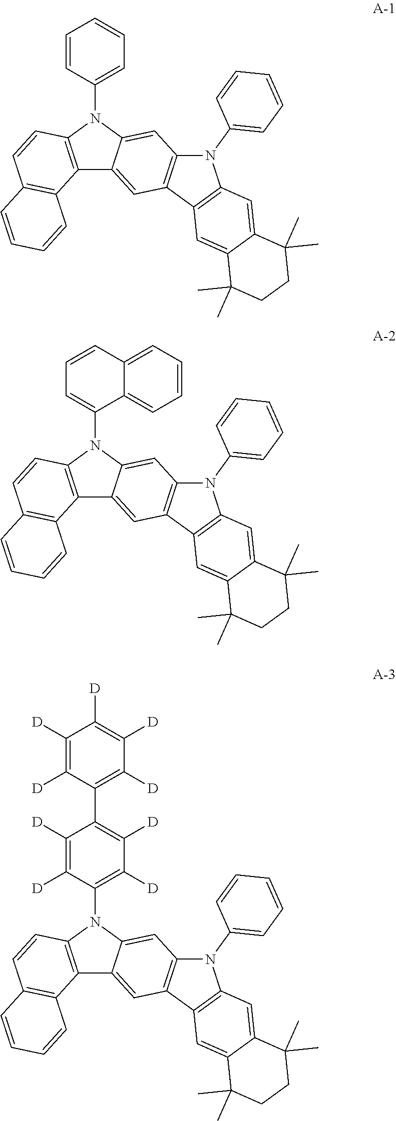 [SMILES: CC1(C)CCC(C)(C)c2cc3c(cc21)c1cc2c4c5ccccc5ccc4n(-c4ccccc4)c2cc1n3-c1ccccc1 ; CC1(C)CCC(C)(C)c2cc3c(cc21)c1cc2c4c5ccccc5ccc4n(-c4cccc5ccccc45)c2cc1n3-c1ccccc1 ; [2H]c1c([2H])c([2H])c(-c2c([2H])c([2H])c(-n3c4cc5c(cc4c4c6ccccc6ccc43)c3cc4c(cc3n5-c3ccccc3)C(C)(C)CCC4(C)C)c([2H])c2[2H])c([2H])c1[2H]]

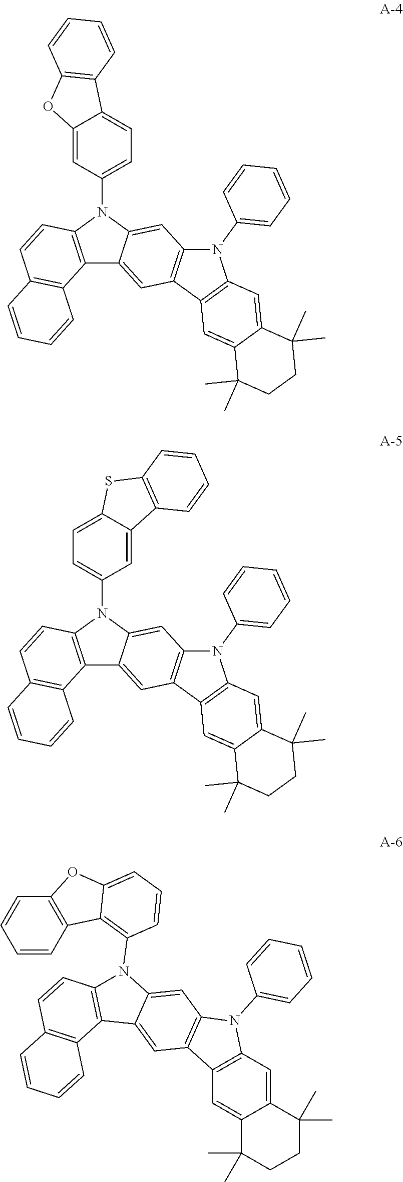 [SMILES: CC1(C)CCC(C)(C)c2cc3c(cc21)c1cc2c4c5ccccc5ccc4n(-c4ccc5c(c4)oc4ccccc45)c2cc1n3-c1ccccc1 ; CC1(C)CCC(C)(C)c2cc3c(cc21)c1cc2c4c5ccccc5ccc4n(-c4ccc5sc6ccccc6c5c4)c2cc1n3-c1ccccc1 ; CC1(C)CCC(C)(C)c2cc3c(cc21)c1cc2c4c5ccccc5ccc4n(-c4cccc5oc6ccccc6c45)c2cc1n3-c1ccccc1]

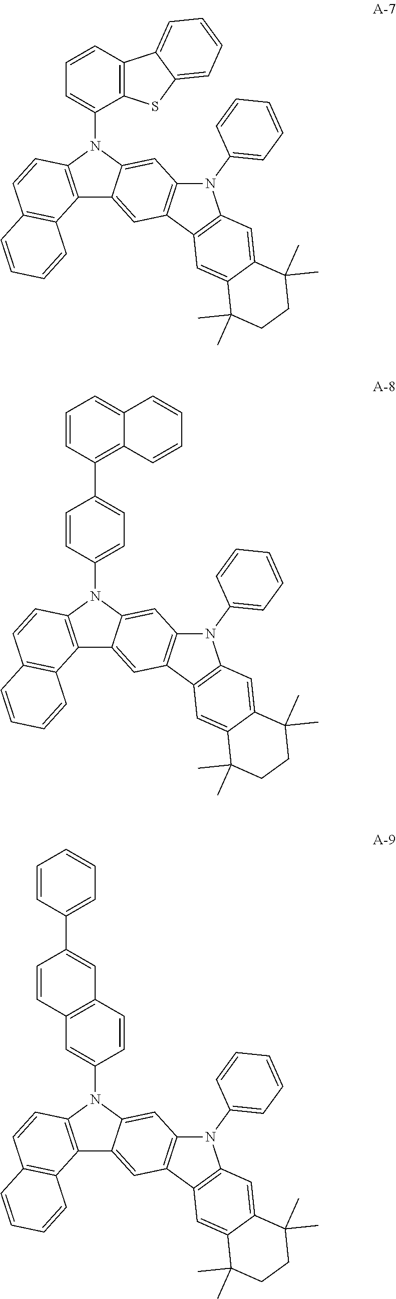 [SMILES: CC1(C)CCC(C)(C)c2cc3c(cc21)c1cc2c4c5ccccc5ccc4n(-c4cccc5c4sc4ccccc45)c2cc1n3-c1ccccc1 ; CC1(C)CCC(C)(C)c2cc3c(cc21)c1cc2c4c5ccccc5ccc4n(-c4ccc(-c5cccc6ccccc56)cc4)c2cc1n3-c1ccccc1 ; CC1(C)CCC(C)(C)c2cc3c(cc21)c1cc2c4c5ccccc5ccc4n(-c4ccc5cc(-c6ccccc6)ccc5c4)c2cc1n3-c1ccccc1]

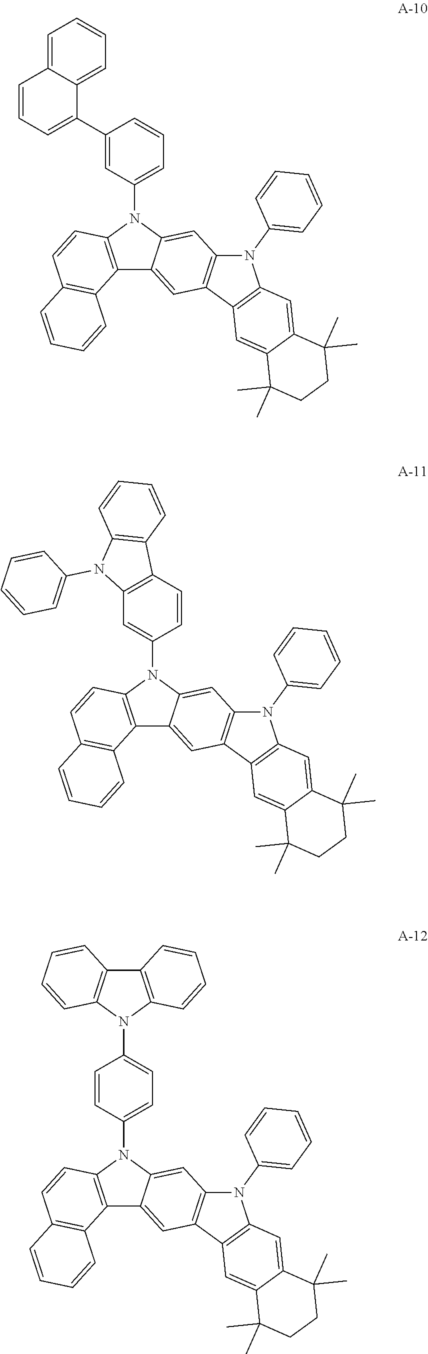 [SMILES: CC1(C)CCC(C)(C)c2cc3c(cc21)c1cc2c4c5ccccc5ccc4n(-c4cccc(-c5cccc6ccccc56)c4)c2cc1n3-c1ccccc1 ; CC1(C)CCC(C)(C)c2cc3c(cc21)c1cc2c4c5ccccc5ccc4n(-c4ccc5c6ccccc6n(-c6ccccc6)c5c4)c2cc1n3-c1ccccc1 ; CC1(C)CCC(C)(C)c2cc3c(cc21)c1cc2c4c5ccccc5ccc4n(-c4ccc(-n5c6ccccc6c6ccccc65)cc4)c2cc1n3-c1ccccc1]

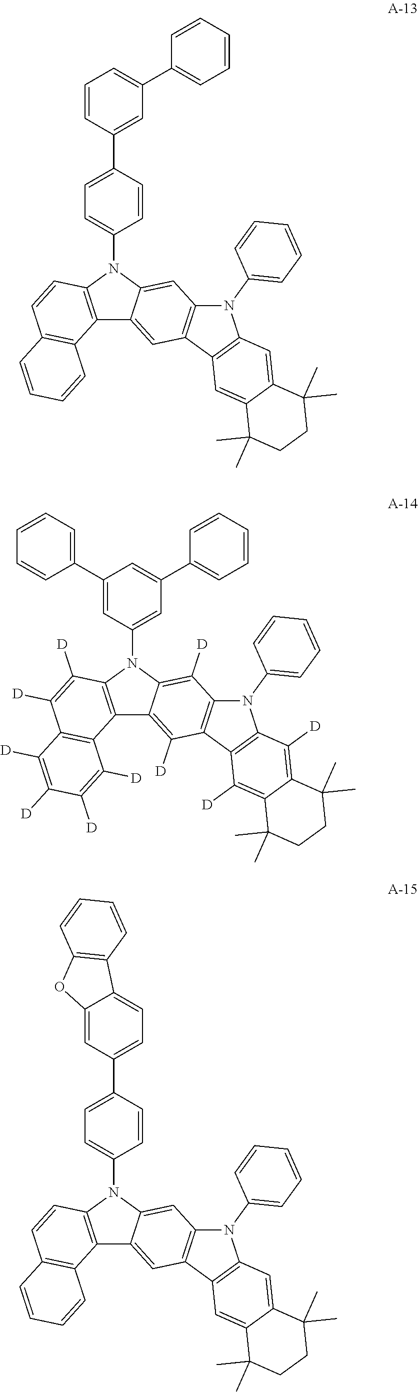 [SMILES: CC1(C)CCC(C)(C)c2cc3c(cc21)c1cc2c4c5ccccc5ccc4n(-c4ccc(-c5cccc(-c6ccccc6)c5)cc4)c2cc1n3-c1ccccc1 ; [2H]c1c([2H])c([2H])c2c(c1[2H])c([2H])c([2H])c1c2c2c([2H])c3c4c([2H])c5c(c([2H])c4n(-c4ccccc4)c3c([2H])c2n1-c1cc(-c2ccccc2)cc(-c2ccccc2)c1)C(C)(C)CCC5(C)C ; CC1(C)CCC(C)(C)c2cc3c(cc21)c1cc2c4c5ccccc5ccc4n(-c4ccc(-c5ccc6c(c5)oc5ccccc56)cc4)c2cc1n3-c1ccccc1]

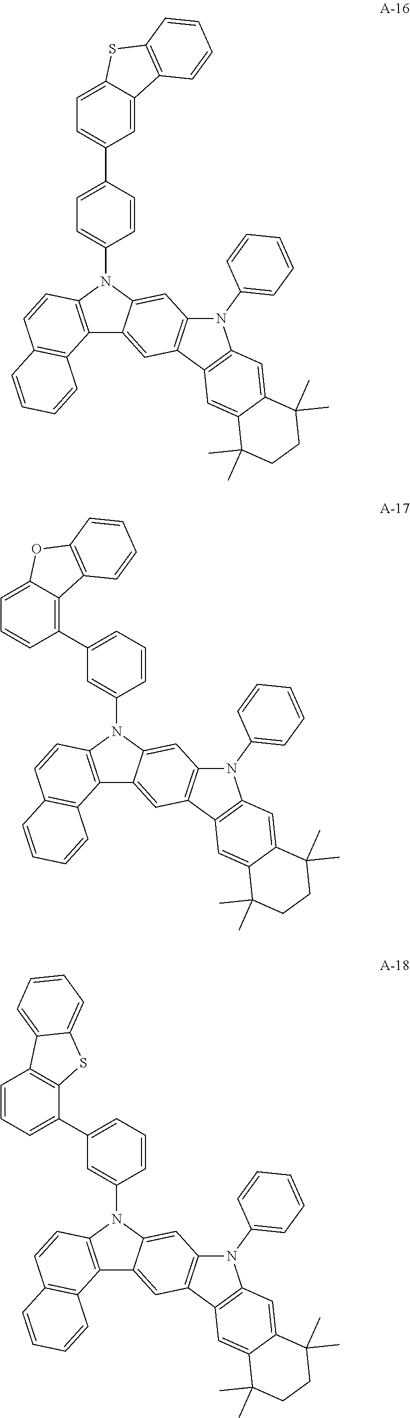 [SMILES: CC1(C)CCC(C)(C)c2cc3c(cc21)c1cc2c4c5ccccc5ccc4n(-c4ccc(-c5ccc6sc7ccccc7c6c5)cc4)c2cc1n3-c1ccccc1 ; CC1(C)CCC(C)(C)c2cc3c(cc21)c1cc2c4c5ccccc5ccc4n(-c4cccc(-c5cccc6oc7ccccc7c56)c4)c2cc1n3-c1ccccc1 ; CC1(C)CCC(C)(C)c2cc3c(cc21)c1cc2c4c5ccccc5ccc4n(-c4cccc(-c5cccc6c5sc5ccccc56)c4)c2cc1n3-c1ccccc1]

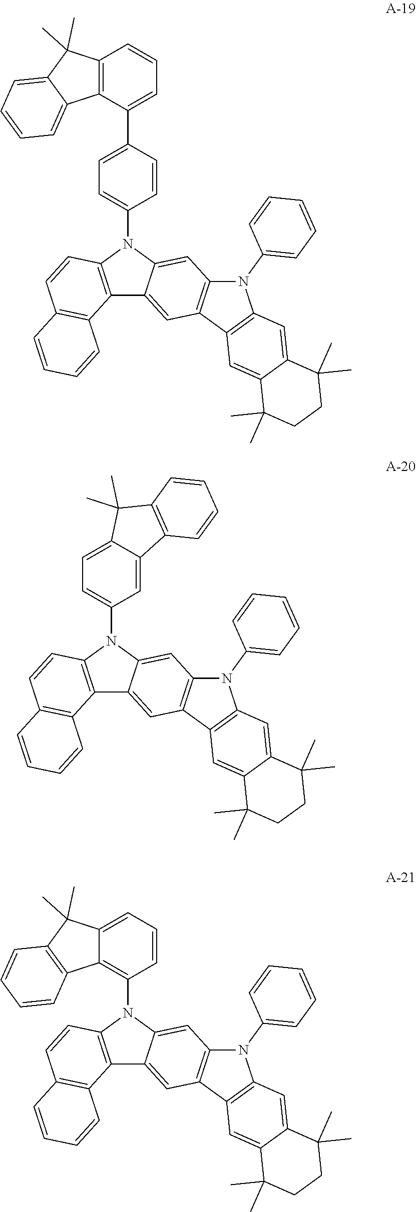 [SMILES: CC1(C)CCC(C)(C)c2cc3c(cc21)c1cc2c4c5ccccc5ccc4n(-c4ccc(-c5cccc6c5-c5ccccc5C6(C)C)cc4)c2cc1n3-c1ccccc1 ; CC1(C)CCC(C)(C)c2cc3c(cc21)c1cc2c4c5ccccc5ccc4n(-c4ccc5c(c4)-c4ccccc4C5(C)C)c2cc1n3-c1ccccc1 ; CC1(C)CCC(C)(C)c2cc3c(cc21)c1cc2c4c5ccccc5ccc4n(-c4cccc5c4-c4ccccc4C5(C)C)c2cc1n3-c1ccccc1]

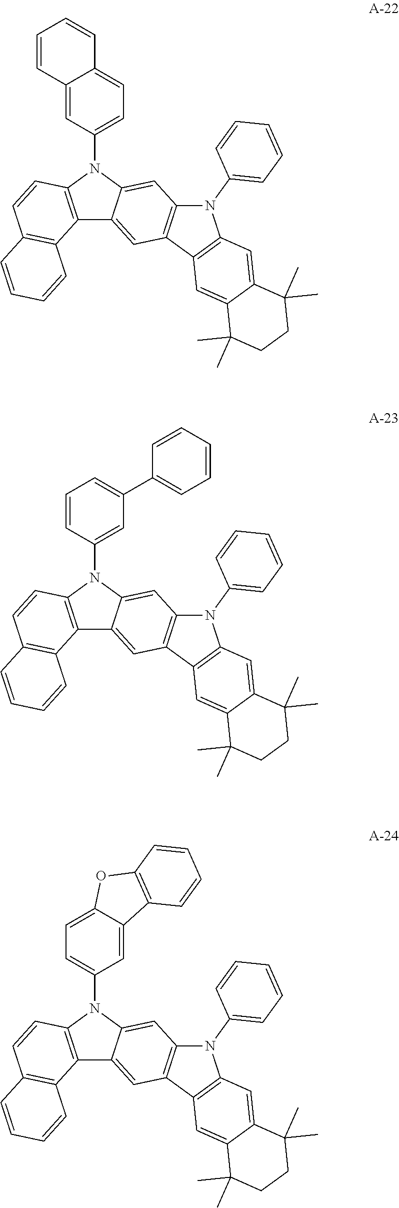 [SMILES: CC1(C)CCC(C)(C)c2cc3c(cc21)c1cc2c4c5ccccc5ccc4n(-c4ccc5ccccc5c4)c2cc1n3-c1ccccc1 ; CC1(C)CCC(C)(C)c2cc3c(cc21)c1cc2c4c5ccccc5ccc4n(-c4cccc(-c5ccccc5)c4)c2cc1n3-c1ccccc1 ; CC1(C)CCC(C)(C)c2cc3c(cc21)c1cc2c4c5ccccc5ccc4n(-c4ccc5oc6ccccc6c5c4)c2cc1n3-c1ccccc1]

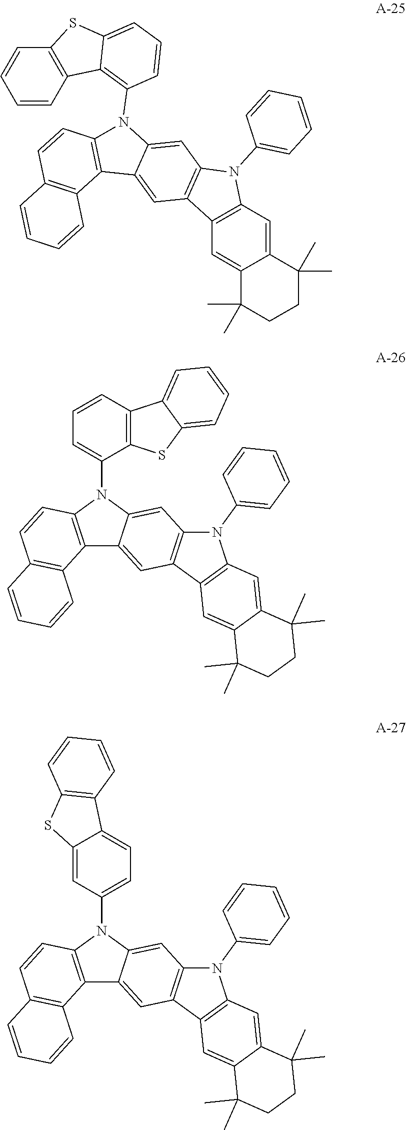

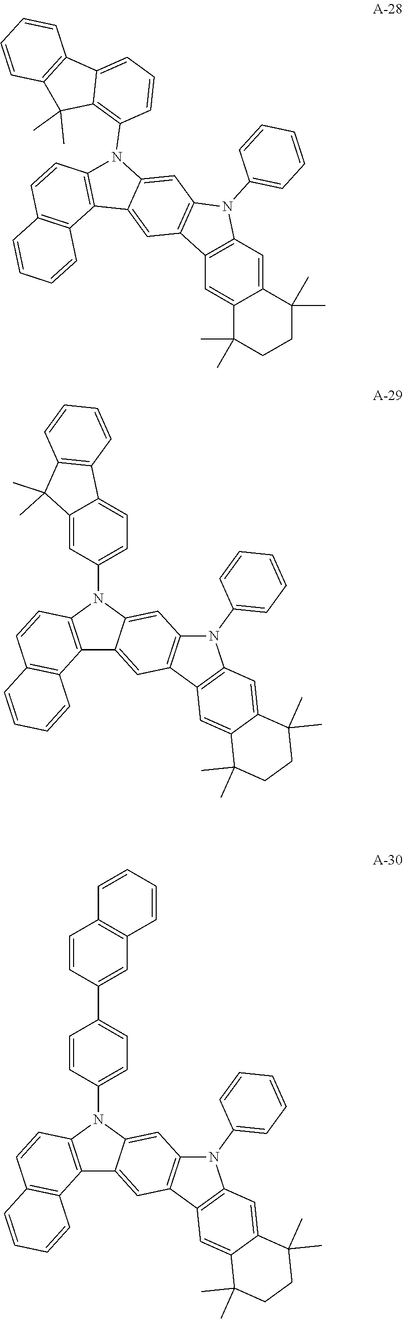 [SMILES: CC1(C)CCC(C)(C)c2cc3c(cc21)c1cc2c4c5ccccc5ccc4n(-c4cccc5c4C(C)(C)c4ccccc4-5)c2cc1n3-c1ccccc1 ; CC1(C)CCC(C)(C)c2cc3c(cc21)c1cc2c4c5ccccc5ccc4n(-c4ccc5c(c4)C(C)(C)c4ccccc4-5)c2cc1n3-c1ccccc1 ; CC1(C)CCC(C)(C)c2cc3c(cc21)c1cc2c4c5ccccc5ccc4n(-c4ccc(-c5ccc6ccccc6c5)cc4)c2cc1n3-c1ccccc1]

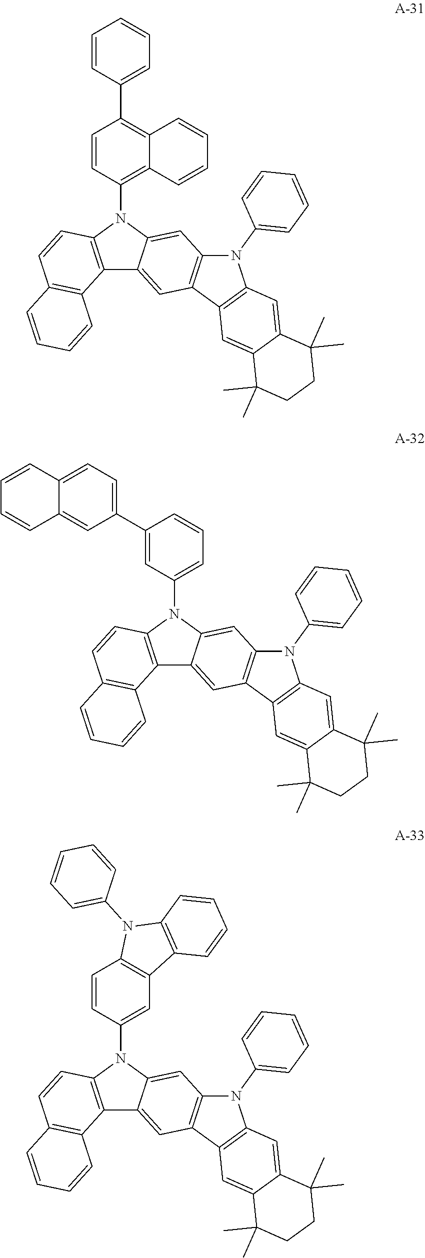 [SMILES: CC1(C)CCC(C)(C)c2cc3c(cc21)c1cc2c4c5ccccc5ccc4n(-c4ccc(-c5ccccc5)c5ccccc45)c2cc1n3-c1ccccc1 ; CC1(C)CCC(C)(C)c2cc3c(cc21)c1cc2c4c5ccccc5ccc4n(-c4cccc(-c5ccc6ccccc6c5)c4)c2cc1n3-c1ccccc1 ; CC1(C)CCC(C)(C)c2cc3c(cc21)c1cc2c4c5ccccc5ccc4n(-c4ccc5c(c4)c4ccccc4n5-c4ccccc4)c2cc1n3-c1ccccc1]

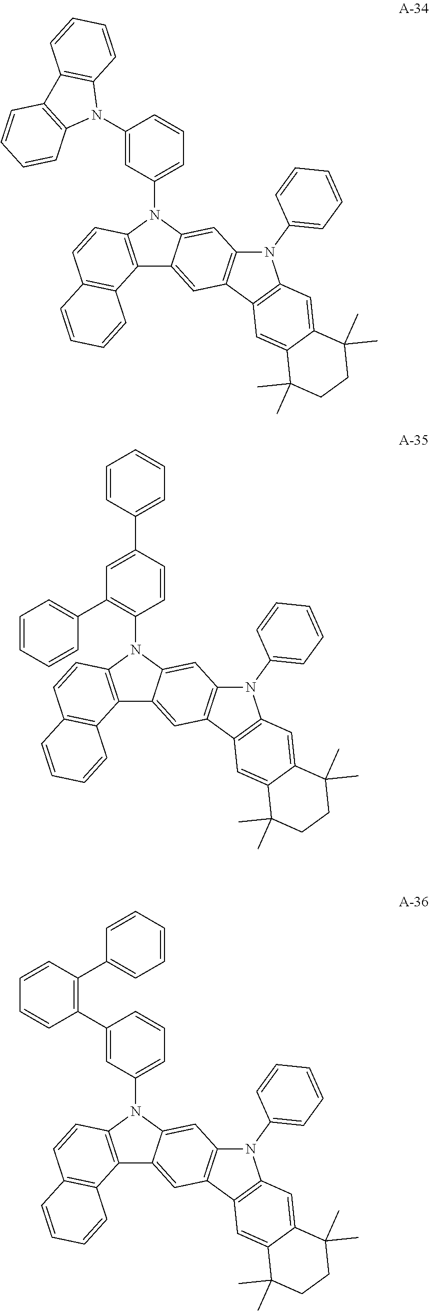 [SMILES: CC1(C)CCC(C)(C)c2cc3c(cc21)c1cc2c4c5ccccc5ccc4n(-c4cccc(-n5c6ccccc6c6ccccc65)c4)c2cc1n3-c1ccccc1 ; CC1(C)CCC(C)(C)c2cc3c(cc21)c1cc2c4c5ccccc5ccc4n(-c4ccc(-c5ccccc5)cc4-c4ccccc4)c2cc1n3-c1ccccc1 ; CC1(C)CCC(C)(C)c2cc3c(cc21)c1cc2c4c5ccccc5ccc4n(-c4cccc(-c5ccccc5-c5ccccc5)c4)c2cc1n3-c1ccccc1]

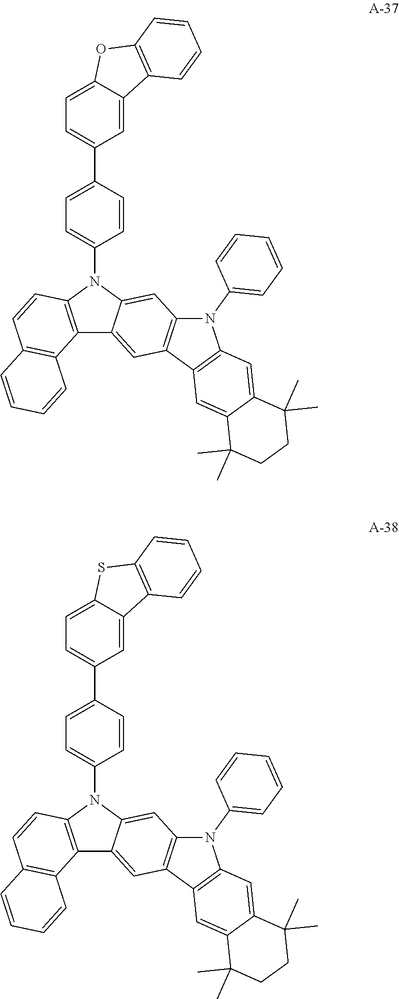 [SMILES: CC1(C)CCC(C)(C)c2cc3c(cc21)c1cc2c4c5ccccc5ccc4n(-c4ccc(-c5ccc6oc7ccccc7c6c5)cc4)c2cc1n3-c1ccccc1 ; CC1(C)CCC(C)(C)c2cc3c(cc21)c1cc2c4c5ccccc5ccc4n(-c4ccc(-c5ccc6sc7ccccc7c6c5)cc4)c2cc1n3-c1ccccc1]

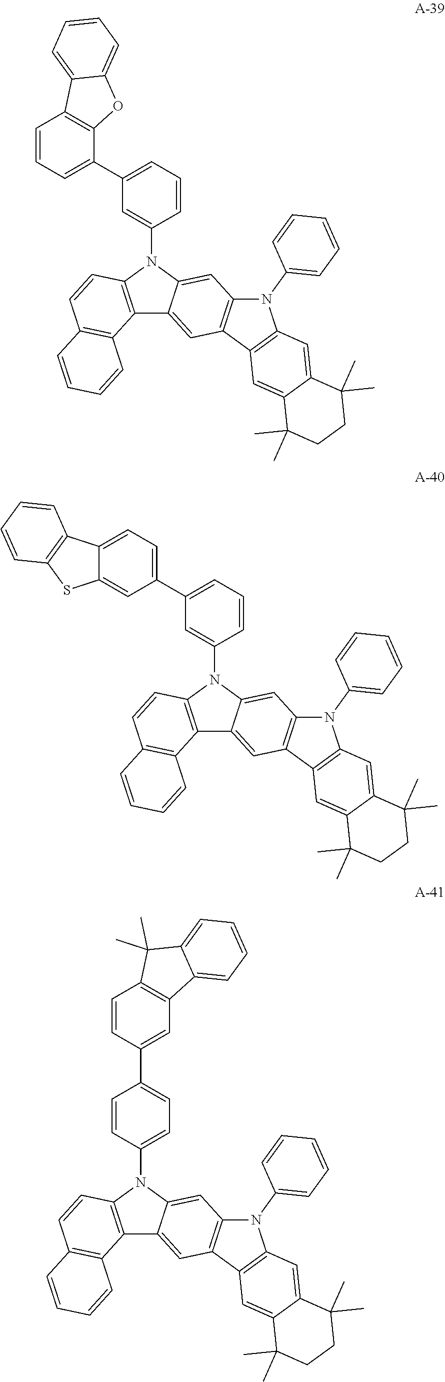 [SMILES: CC1(C)CCC(C)(C)c2cc3c(cc21)c1cc2c4c5ccccc5ccc4n(-c4cccc(-c5cccc6c5oc5ccccc56)c4)c2cc1n3-c1ccccc1 ; CC1(C)CCC(C)(C)c2cc3c(cc21)c1cc2c4c5ccccc5ccc4n(-c4cccc(-c5ccc6c(c5)sc5ccccc56)c4)c2cc1n3-c1ccccc1 ; CC1(C)CCC(C)(C)c2cc3c(cc21)c1cc2c4c5ccccc5ccc4n(-c4ccc(-c5ccc6c(c5)-c5ccccc5C6(C)C)cc4)c2cc1n3-c1ccccc1]

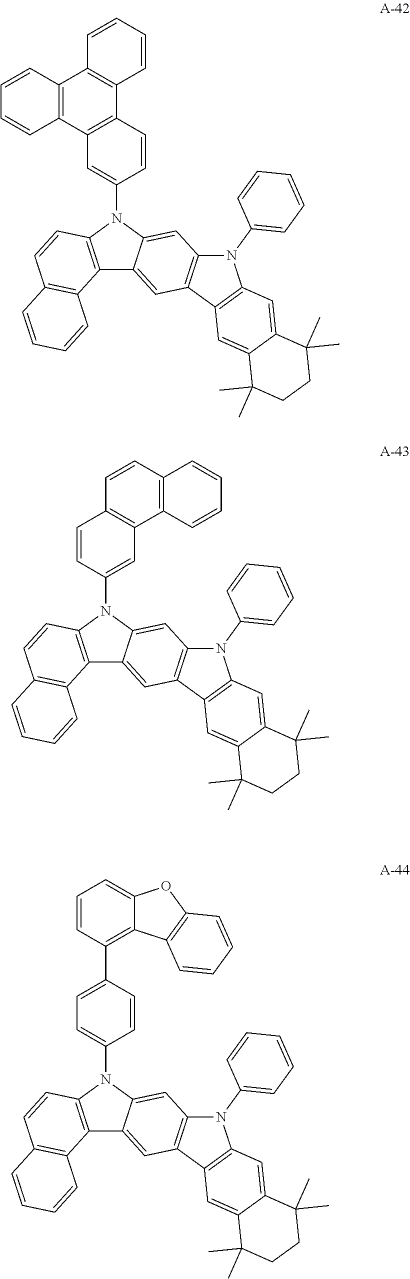 [SMILES: CC1(C)CCC(C)(C)c2cc3c(cc21)c1cc2c4c5ccccc5ccc4n(-c4ccc5c6ccccc6c6ccccc6c5c4)c2cc1n3-c1ccccc1 ; CC1(C)CCC(C)(C)c2cc3c(cc21)c1cc2c4c5ccccc5ccc4n(-c4ccc5ccc6ccccc6c5c4)c2cc1n3-c1ccccc1 ; CC1(C)CCC(C)(C)c2cc3c(cc21)c1cc2c4c5ccccc5ccc4n(-c4ccc(-c5cccc6oc7ccccc7c56)cc4)c2cc1n3-c1ccccc1]

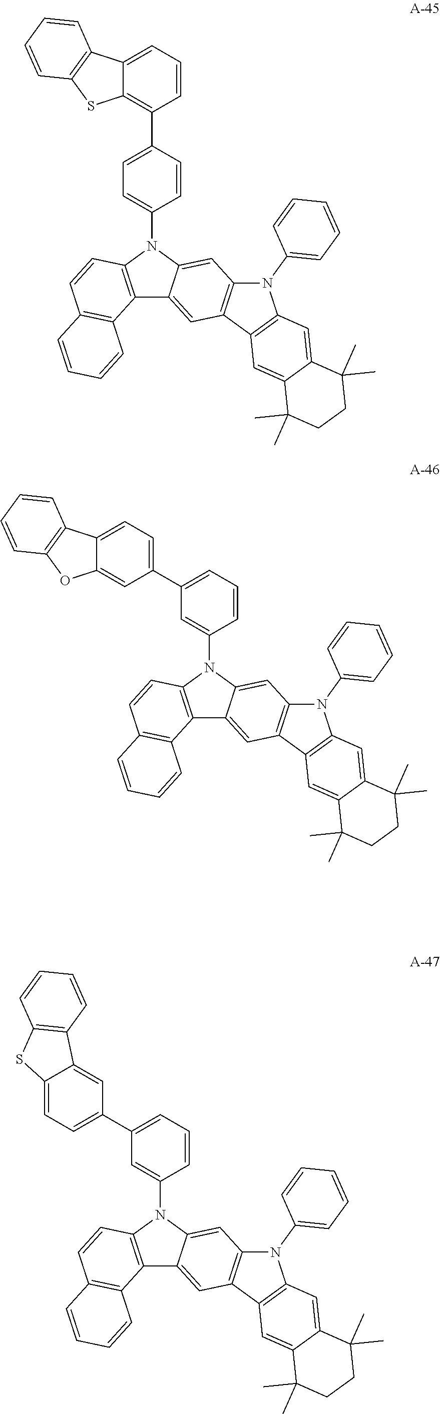 [SMILES: CC1(C)CCC(C)(C)c2cc3c(cc21)c1cc2c4c5ccccc5ccc4n(-c4ccc(-c5cccc6c5sc5ccccc56)cc4)c2cc1n3-c1ccccc1 ; CC1(C)CCC(C)(C)c2cc3c(cc21)c1cc2c4c5ccccc5ccc4n(-c4cccc(-c5ccc6c(c5)oc5ccccc56)c4)c2cc1n3-c1ccccc1 ; CC1(C)CCC(C)(C)c2cc3c(cc21)c1cc2c4c5ccccc5ccc4n(-c4cccc(-c5ccc6sc7ccccc7c6c5)c4)c2cc1n3-c1ccccc1]

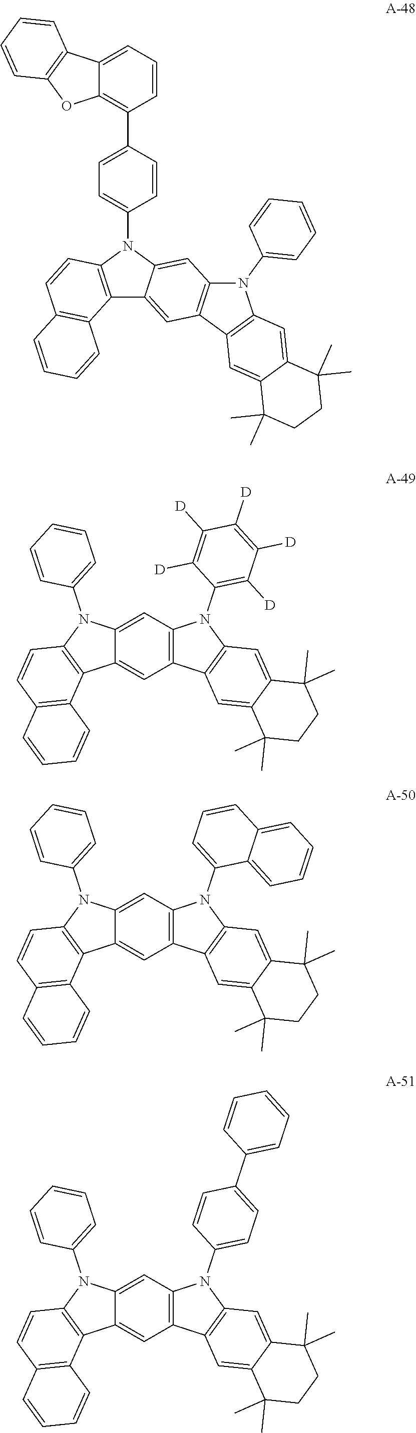

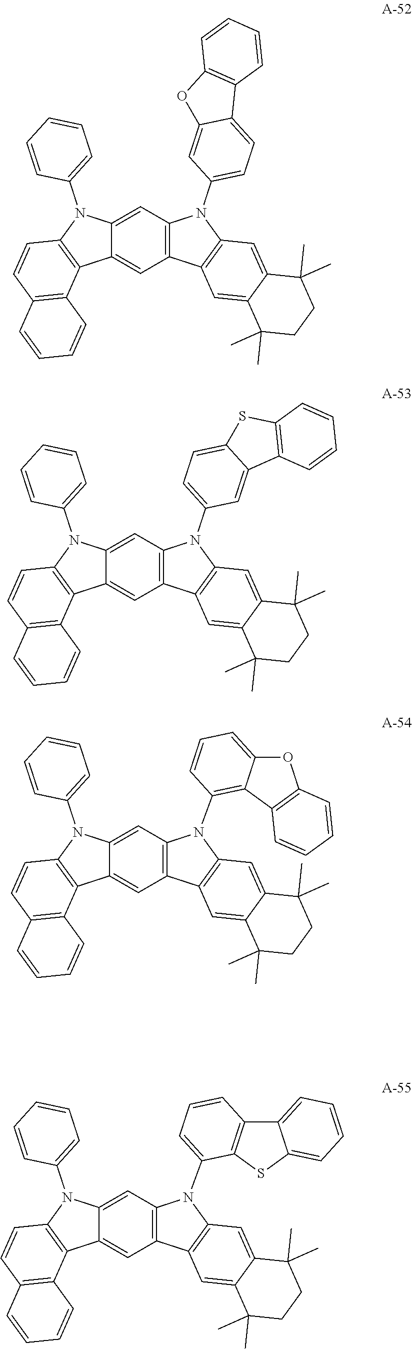

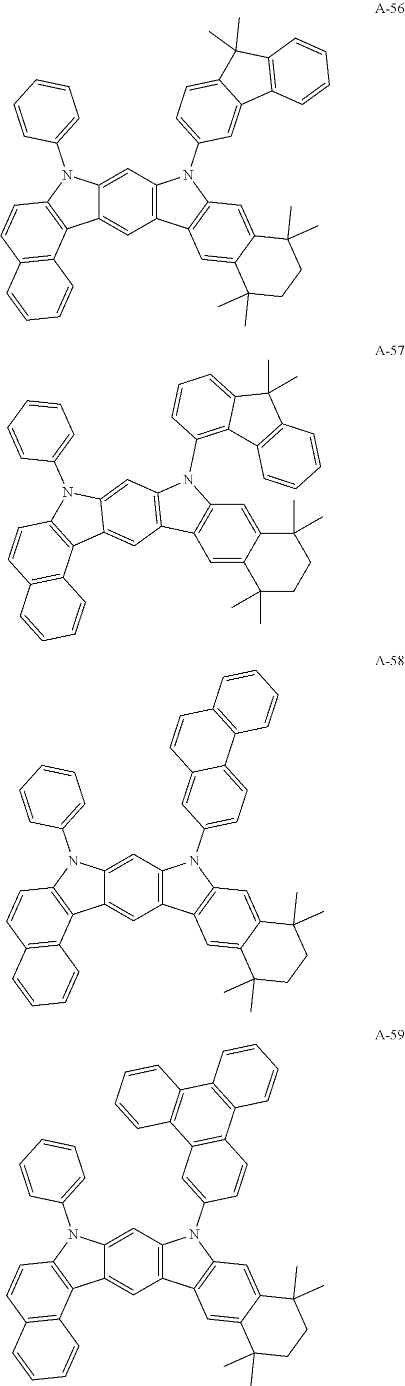

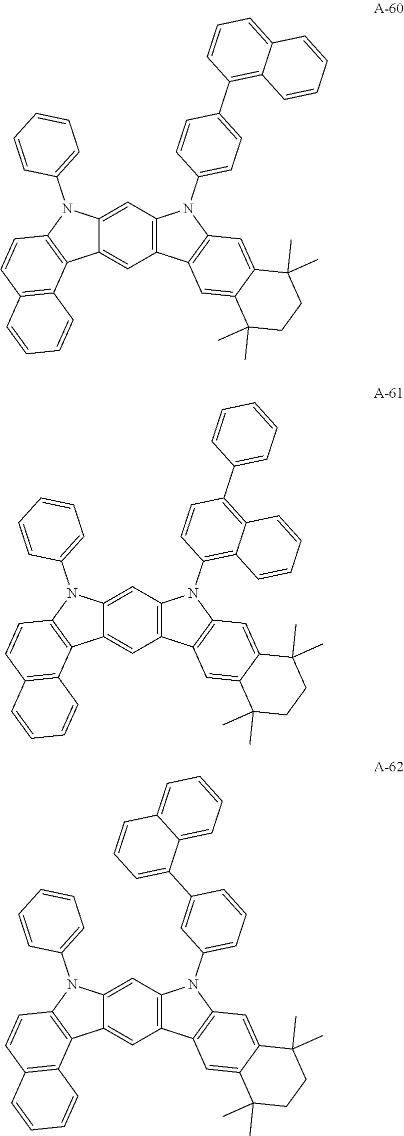

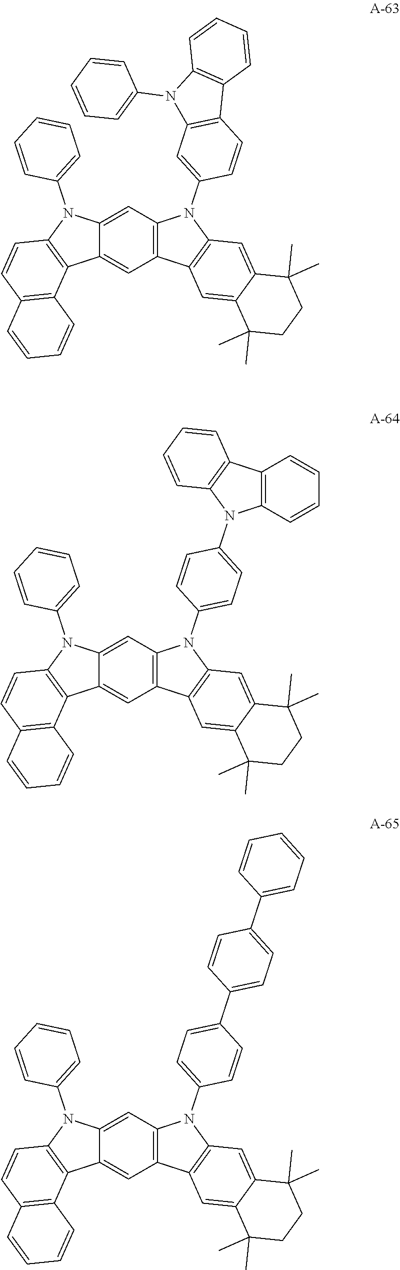

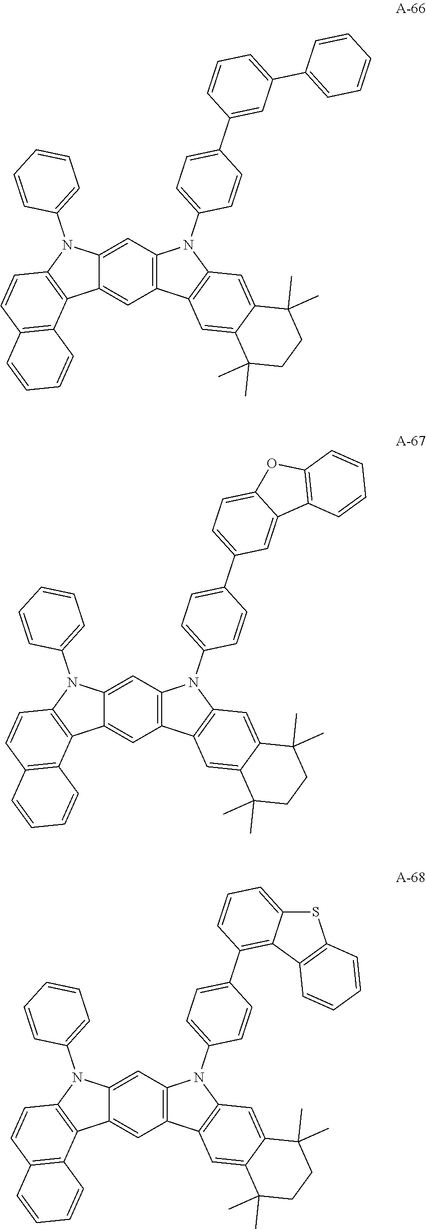 [SMILES: CC1(C)CCC(C)(C)c2cc3c(cc21)c1cc2c4c5ccccc5ccc4n(-c4ccccc4)c2cc1n3-c1ccc(-c2cccc(-c3ccccc3)c2)cc1 ; CC1(C)CCC(C)(C)c2cc3c(cc21)c1cc2c4c5ccccc5ccc4n(-c4ccccc4)c2cc1n3-c1ccc(-c2ccc3oc4ccccc4c3c2)cc1 ; CC1(C)CCC(C)(C)c2cc3c(cc21)c1cc2c4c5ccccc5ccc4n(-c4ccccc4)c2cc1n3-c1ccc(-c2cccc3sc4ccccc4c23)cc1]

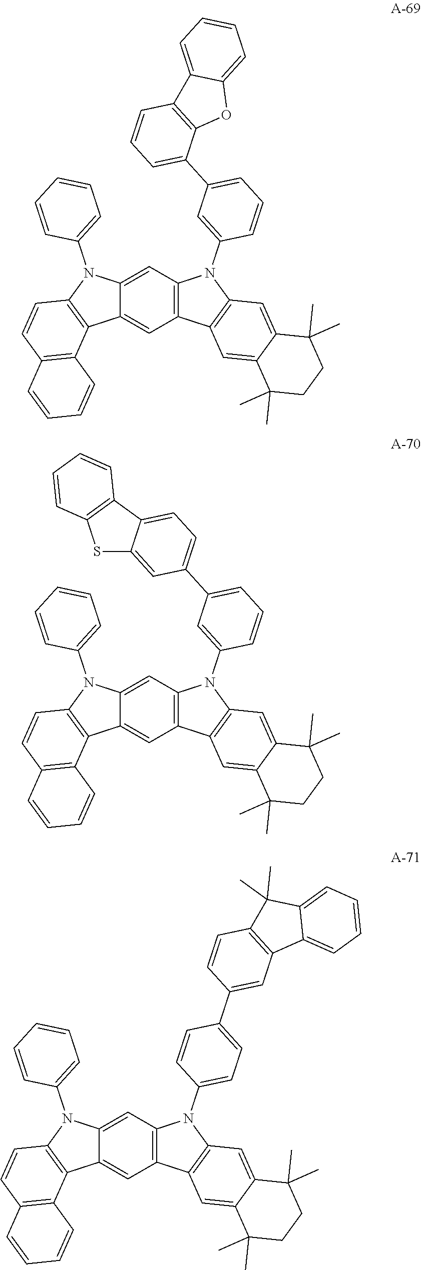

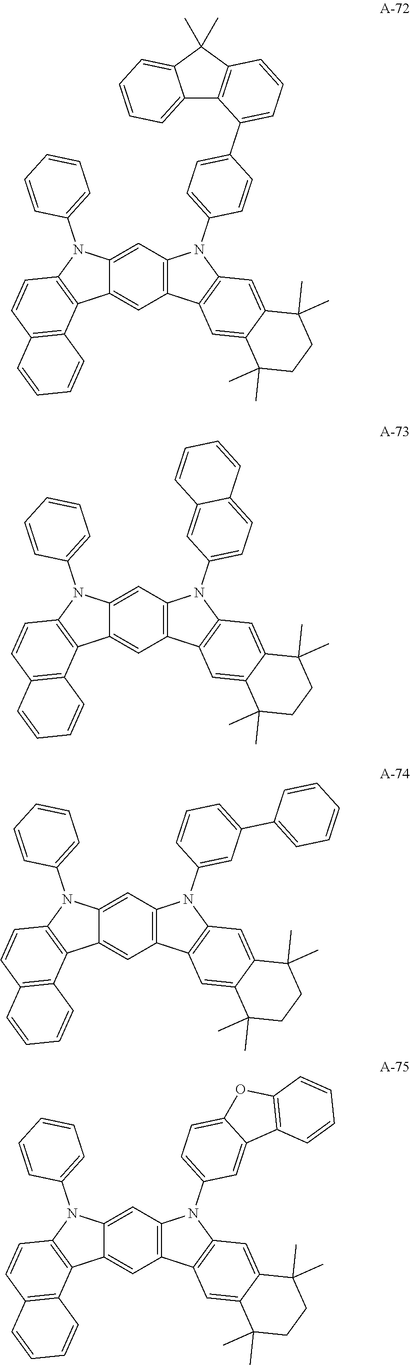

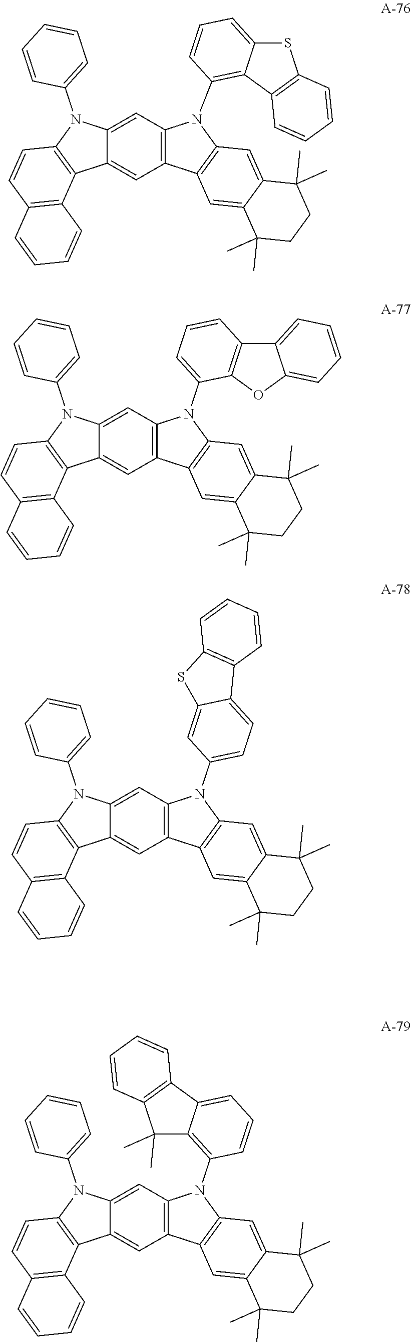

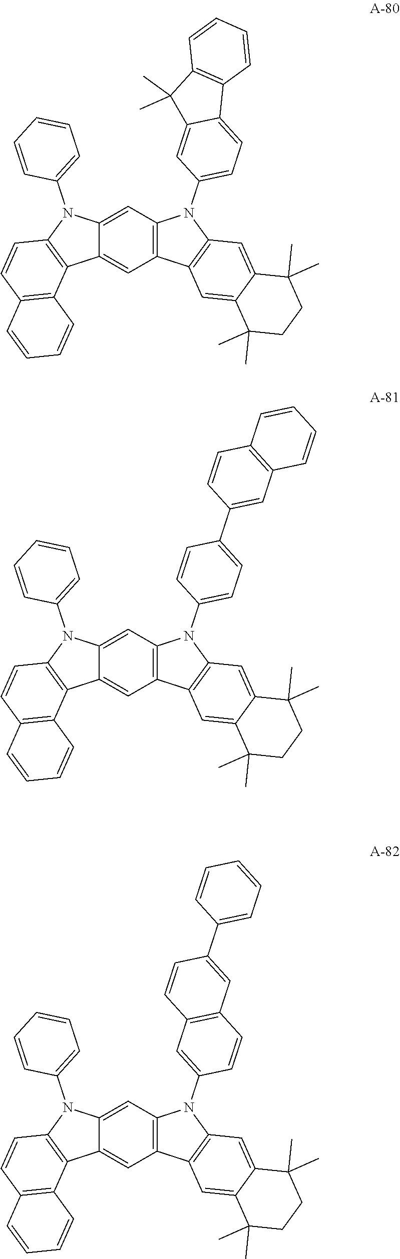 [SMILES: CC1(C)CCC(C)(C)c2cc3c(cc21)c1cc2c4c5ccccc5ccc4n(-c4ccccc4)c2cc1n3-c1ccc2c(c1)C(C)(C)c1ccccc1-2 ; CC1(C)CCC(C)(C)c2cc3c(cc21)c1cc2c4c5ccccc5ccc4n(-c4ccccc4)c2cc1n3-c1ccc(-c2ccc3ccccc3c2)cc1 ; CC1(C)CCC(C)(C)c2cc3c(cc21)c1cc2c4c5ccccc5ccc4n(-c4ccccc4)c2cc1n3-c1ccc2cc(-c3ccccc3)ccc2c1]

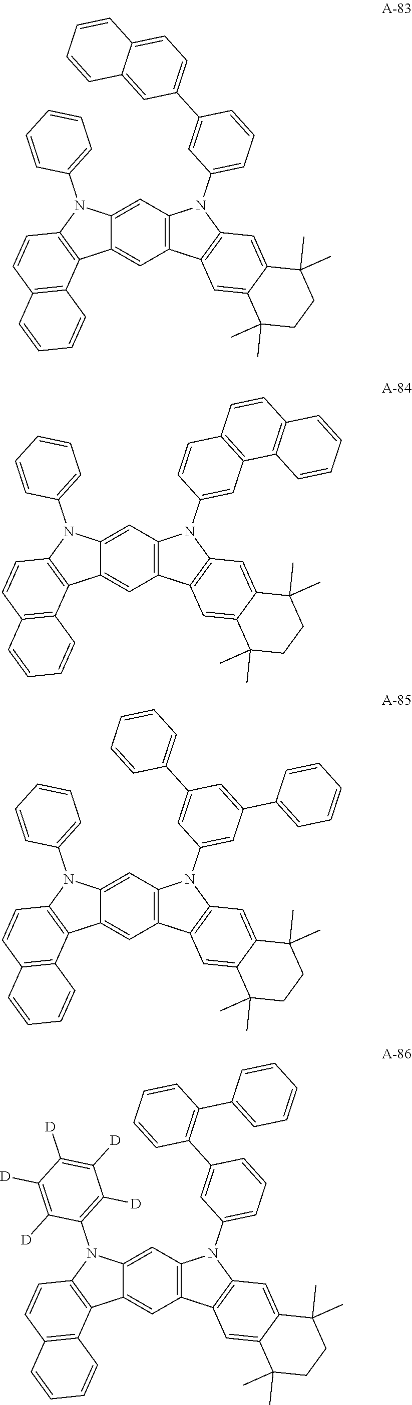

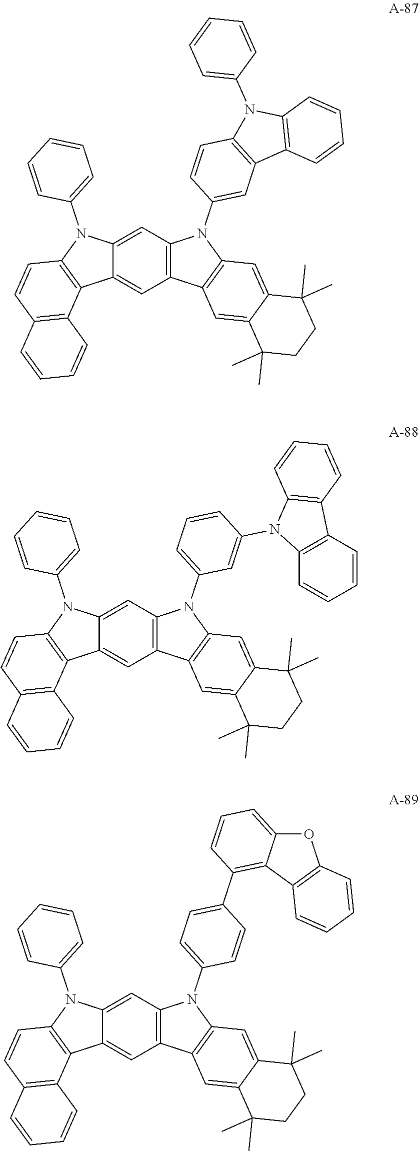 [SMILES: CC1(C)CCC(C)(C)c2cc3c(cc21)c1cc2c4c5ccccc5ccc4n(-c4ccccc4)c2cc1n3-c1ccc2c(c1)c1ccccc1n2-c1ccccc1 ; CC1(C)CCC(C)(C)c2cc3c(cc21)c1cc2c4c5ccccc5ccc4n(-c4ccccc4)c2cc1n3-c1cccc(-n2c3ccccc3c3ccccc32)c1 ; CC1(C)CCC(C)(C)c2cc3c(cc21)c1cc2c4c5ccccc5ccc4n(-c4ccccc4)c2cc1n3-c1ccc(-c2cccc3oc4ccccc4c23)cc1]

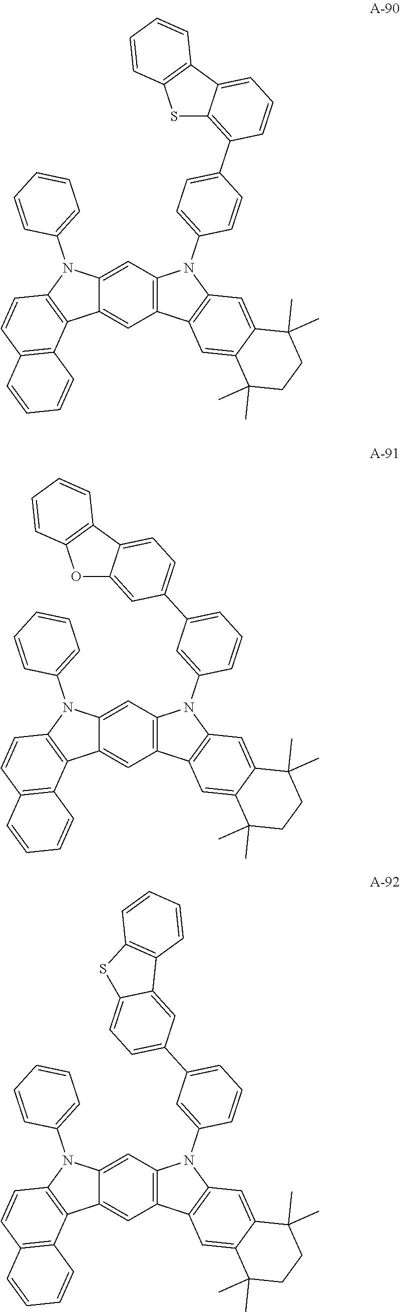 [SMILES: CC1(C)CCC(C)(C)c2cc3c(cc21)c1cc2c4c5ccccc5ccc4n(-c4ccccc4)c2cc1n3-c1ccc(-c2cccc3c2sc2ccccc23)cc1 ; CC1(C)CCC(C)(C)c2cc3c(cc21)c1cc2c4c5ccccc5ccc4n(-c4ccccc4)c2cc1n3-c1cccc(-c2ccc3c(c2)oc2ccccc23)c1 ; CC1(C)CCC(C)(C)c2cc3c(cc21)c1cc2c4c5ccccc5ccc4n(-c4ccccc4)c2cc1n3-c1cccc(-c2ccc3sc4ccccc4c3c2)c1]

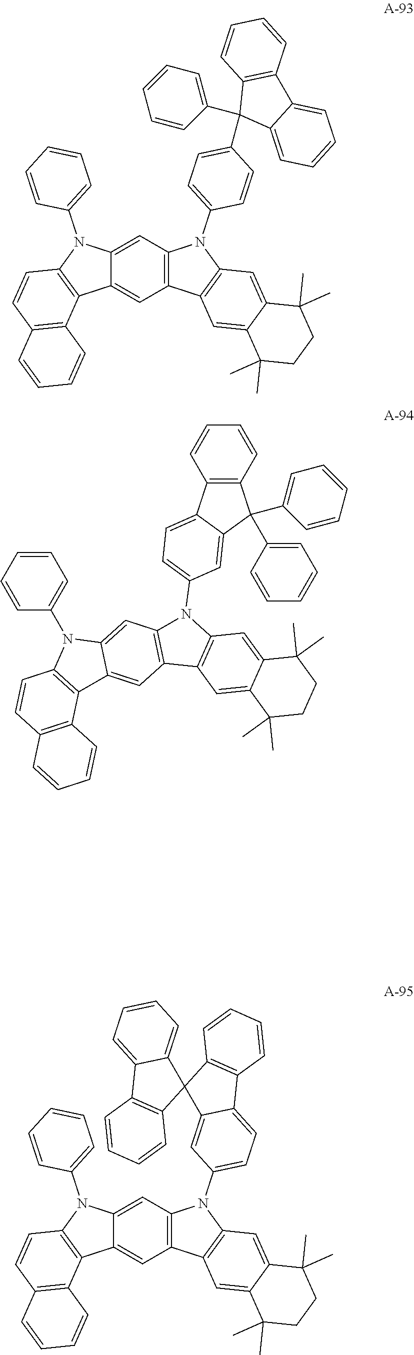

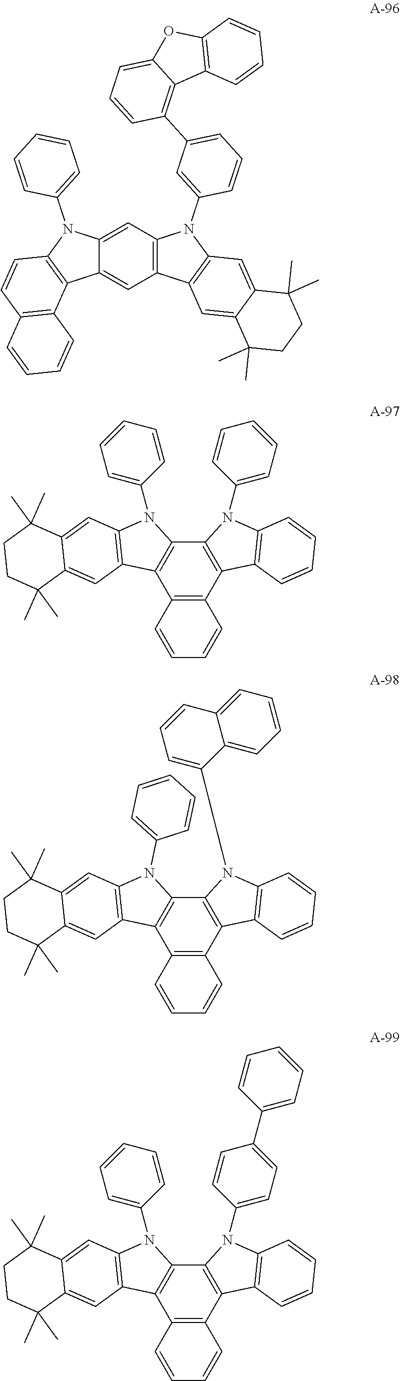

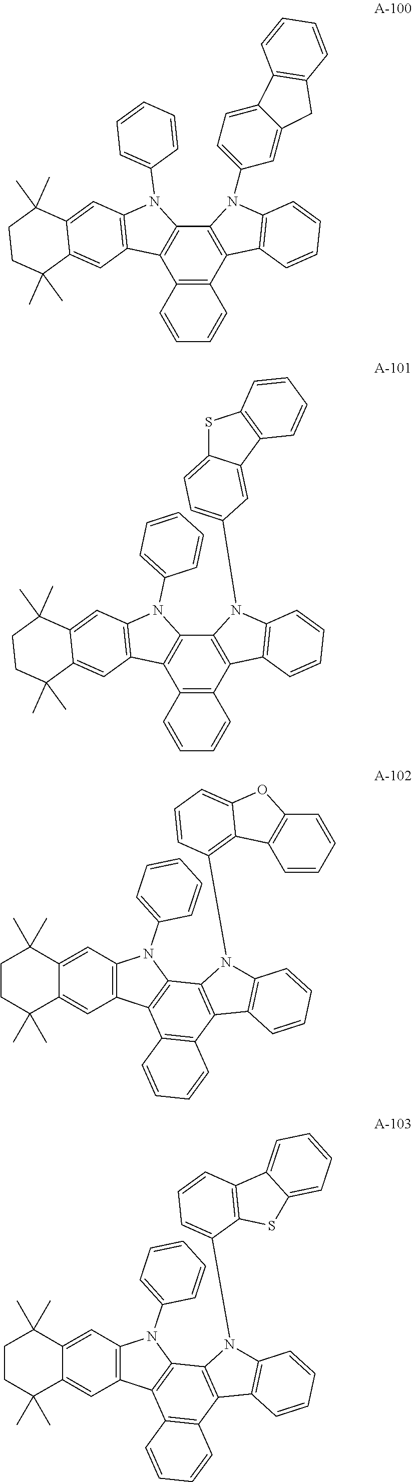

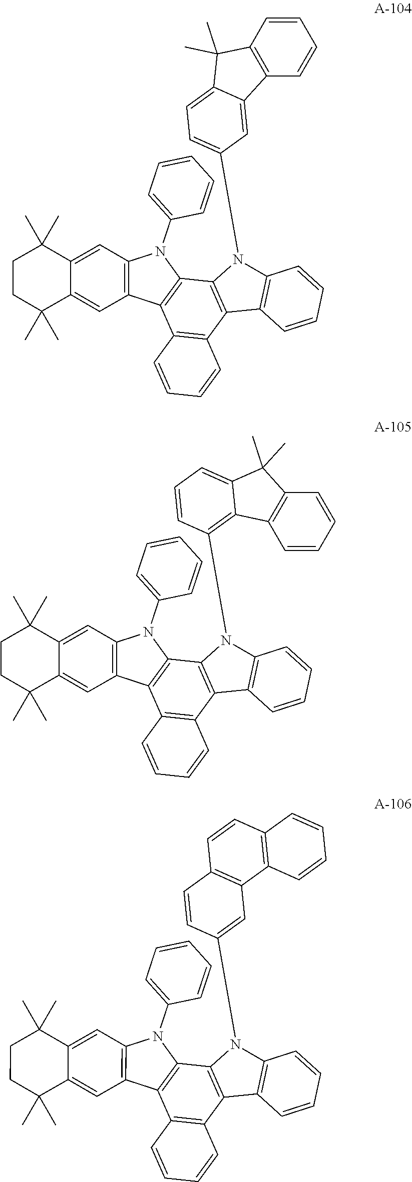 [SMILES: CC1(C)CCC(C)(C)c2cc3c(cc21)c1c2ccccc2c2c4ccccc4n(-c4ccc5c(c4)-c4ccccc4C5(C)C)c2c1n3-c1ccccc1 ; CC1(C)CCC(C)(C)c2cc3c(cc21)c1c2ccccc2c2c4ccccc4n(-c4cccc5c4-c4ccccc4C5(C)C)c2c1n3-c1ccccc1 ; CC1(C)CCC(C)(C)c2cc3c(cc21)c1c2ccccc2c2c4ccccc4n(-c4ccc5ccc6ccccc6c5c4)c2c1n3-c1ccccc1]

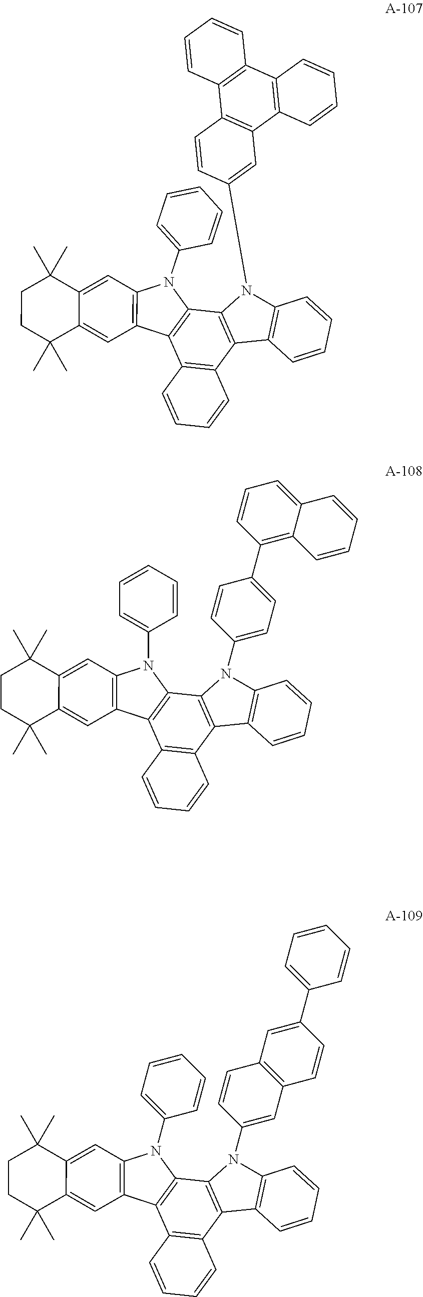 [SMILES: CC1(C)CCC(C)(C)c2cc3c(cc21)c1c2ccccc2c2c4ccccc4n(-c4ccc5c6ccccc6c6ccccc6c5c4)c2c1n3-c1ccccc1 ; CC1(C)CCC(C)(C)c2cc3c(cc21)c1c2ccccc2c2c4ccccc4n(-c4ccc(-c5cccc6ccccc56)cc4)c2c1n3-c1ccccc1 ; CC1(C)CCC(C)(C)c2cc3c(cc21)c1c2ccccc2c2c4ccccc4n(-c4ccc5cc(-c6ccccc6)ccc5c4)c2c1n3-c1ccccc1]

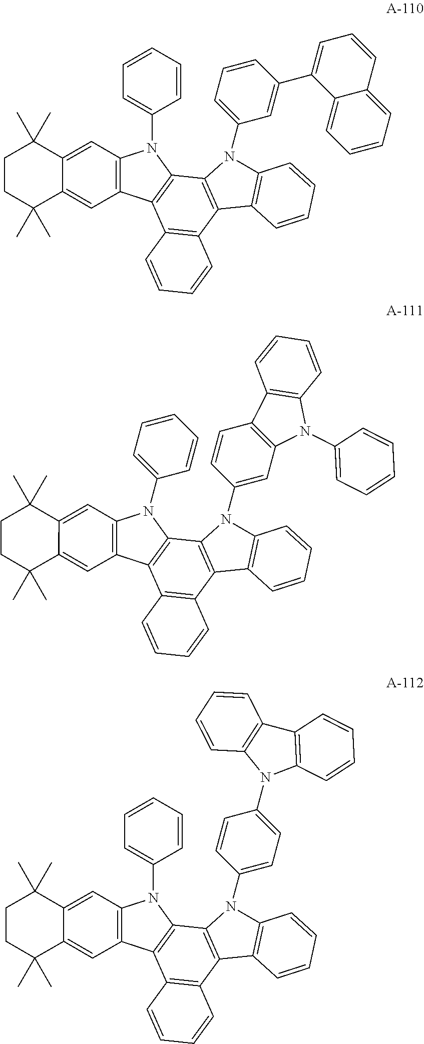

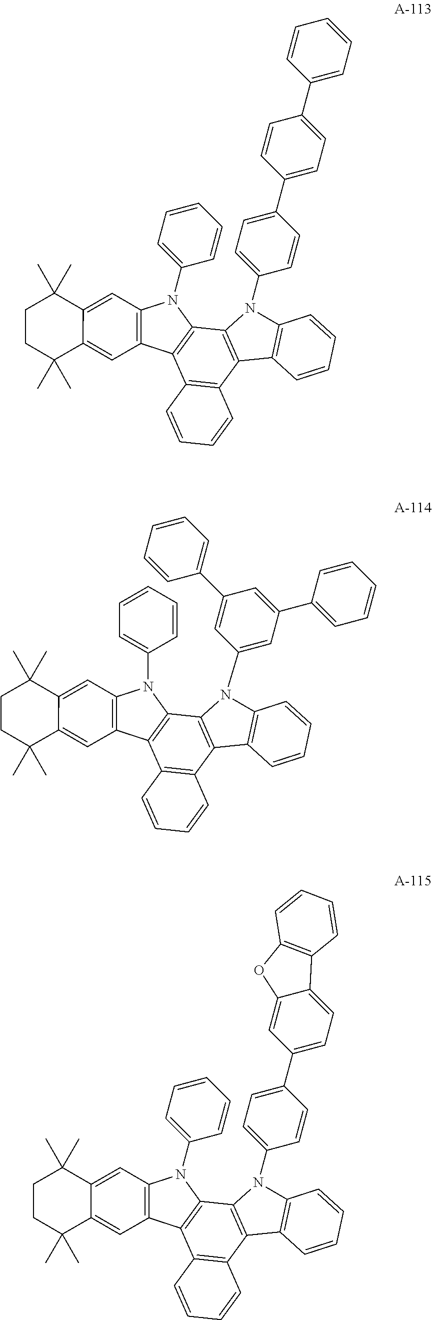 [SMILES: CC1(C)CCC(C)(C)c2cc3c(cc21)c1c2ccccc2c2c4ccccc4n(-c4ccc(-c5ccc(-c6ccccc6)cc5)cc4)c2c1n3-c1ccccc1 ; CC1(C)CCC(C)(C)c2cc3c(cc21)c1c2ccccc2c2c4ccccc4n(-c4cc(-c5ccccc5)cc(-c5ccccc5)c4)c2c1n3-c1ccccc1 ; CC1(C)CCC(C)(C)c2cc3c(cc21)c1c2ccccc2c2c4ccccc4n(-c4ccc(-c5ccc6c(c5)oc5ccccc56)cc4)c2c1n3-c1ccccc1]

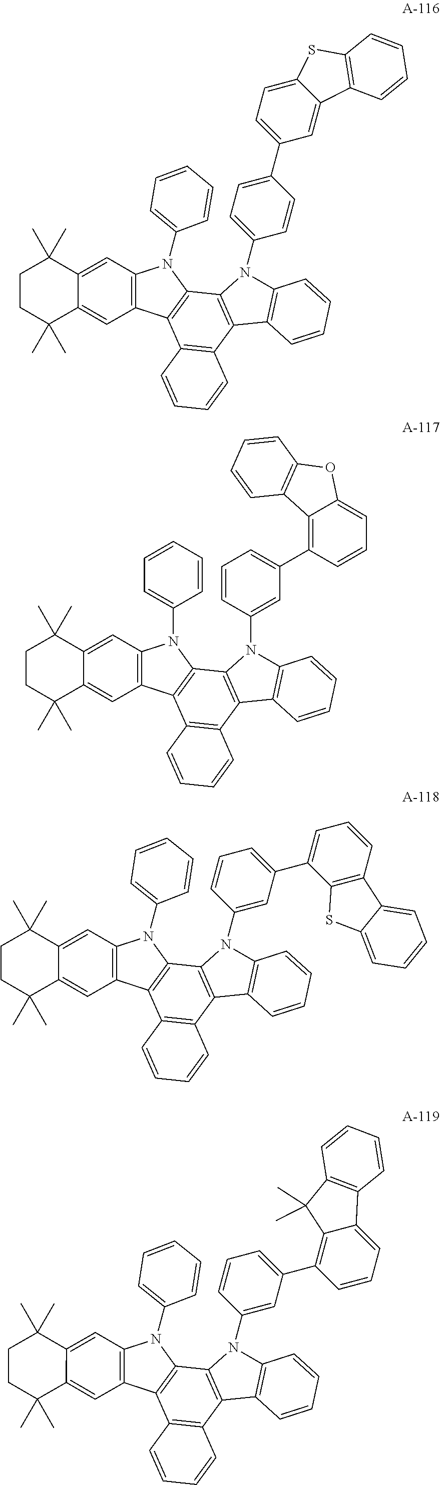 [SMILES: CC1(C)CCC(C)(C)c2cc3c(cc21)c1c2ccccc2c2c4ccccc4n(-c4ccc(-c5ccc6sc7ccccc7c6c5)cc4)c2c1n3-c1ccccc1 ; CC1(C)CCC(C)(C)c2cc3c(cc21)c1c2ccccc2c2c4ccccc4n(-c4cccc(-c5cccc6oc7ccccc7c56)c4)c2c1n3-c1ccccc1 ; CC1(C)CCC(C)(C)c2cc3c(cc21)c1c2ccccc2c2c4ccccc4n(-c4cccc(-c5cccc6c5sc5ccccc56)c4)c2c1n3-c1ccccc1 ; CC1(C)CCC(C)(C)c2cc3c(cc21)c1c2ccccc2c2c4ccccc4n(-c4cccc(-c5cccc6c5C(C)(C)c5ccccc5-6)c4)c2c1n3-c1ccccc1]

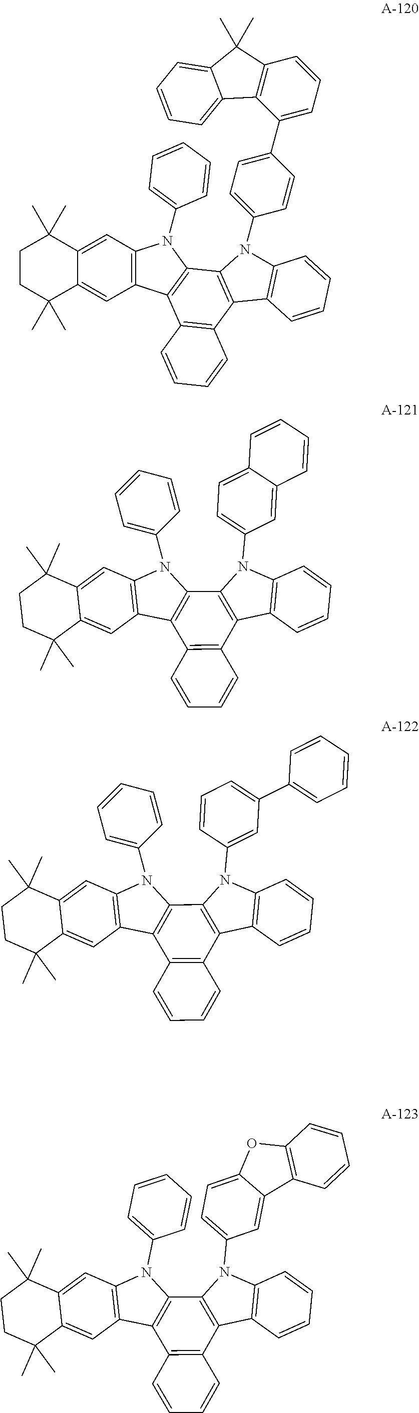 [SMILES: CC1(C)CCC(C)(C)c2cc3c(cc21)c1c2ccccc2c2c4ccccc4n(-c4ccc(-c5cccc6c5-c5ccccc5C6(C)C)cc4)c2c1n3-c1ccccc1 ; CC1(C)CCC(C)(C)c2cc3c(cc21)c1c2ccccc2c2c4ccccc4n(-c4ccc5ccccc5c4)c2c1n3-c1ccccc1 ; CC1(C)CCC(C)(C)c2cc3c(cc21)c1c2ccccc2c2c4ccccc4n(-c4cccc(-c5ccccc5)c4)c2c1n3-c1ccccc1 ; CC1(C)CCC(C)(C)c2cc3c(cc21)c1c2ccccc2c2c4ccccc4n(-c4ccc5oc6ccccc6c5c4)c2c1n3-c1ccccc1]

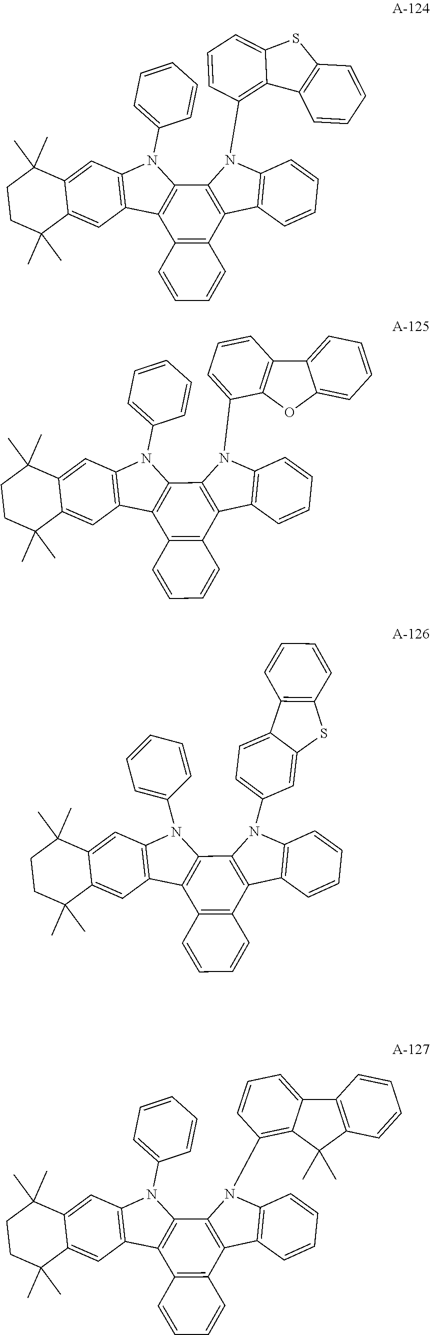

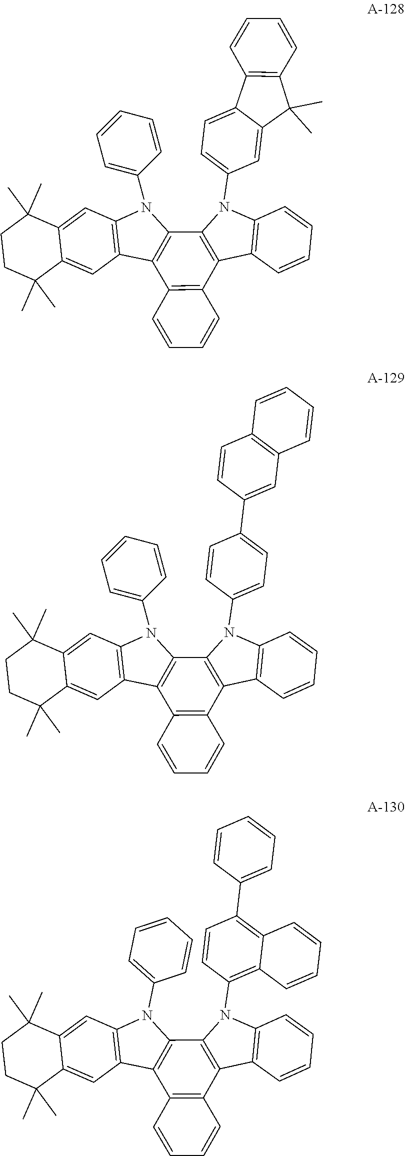 [SMILES: CC1(C)CCC(C)(C)c2cc3c(cc21)c1c2ccccc2c2c4ccccc4n(-c4ccc5c(c4)C(C)(C)c4ccccc4-5)c2c1n3-c1ccccc1 ; CC1(C)CCC(C)(C)c2cc3c(cc21)c1c2ccccc2c2c4ccccc4n(-c4ccc(-c5ccc6ccccc6c5)cc4)c2c1n3-c1ccccc1 ; CC1(C)CCC(C)(C)c2cc3c(cc21)c1c2ccccc2c2c4ccccc4n(-c4ccc(-c5ccccc5)c5ccccc45)c2c1n3-c1ccccc1]

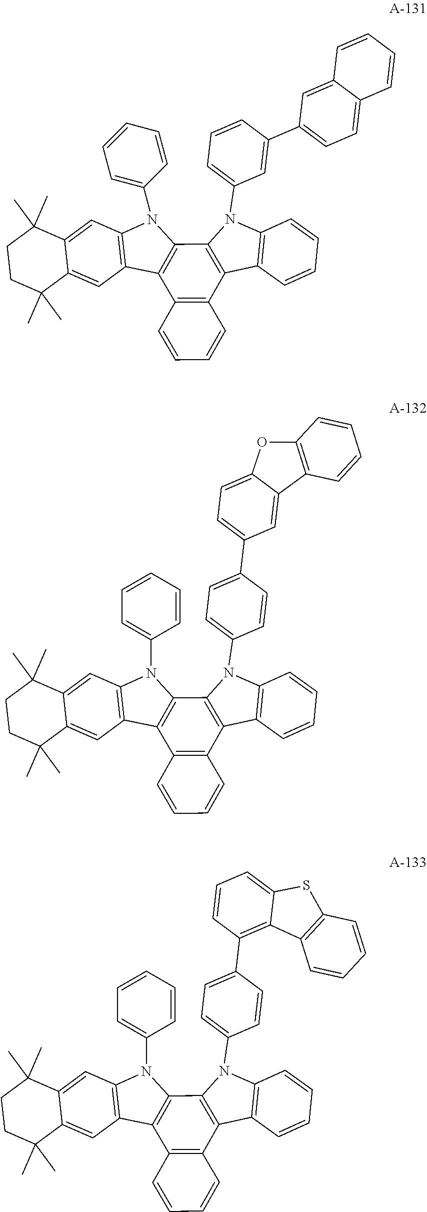 [SMILES: CC1(C)CCC(C)(C)c2cc3c(cc21)c1c2ccccc2c2c4ccccc4n(-c4cccc(-c5ccc6ccccc6c5)c4)c2c1n3-c1ccccc1 ; CC1(C)CCC(C)(C)c2cc3c(cc21)c1c2ccccc2c2c4ccccc4n(-c4ccc(-c5ccc6oc7ccccc7c6c5)cc4)c2c1n3-c1ccccc1 ; CC1(C)CCC(C)(C)c2cc3c(cc21)c1c2ccccc2c2c4ccccc4n(-c4ccc(-c5cccc6sc7ccccc7c56)cc4)c2c1n3-c1ccccc1]

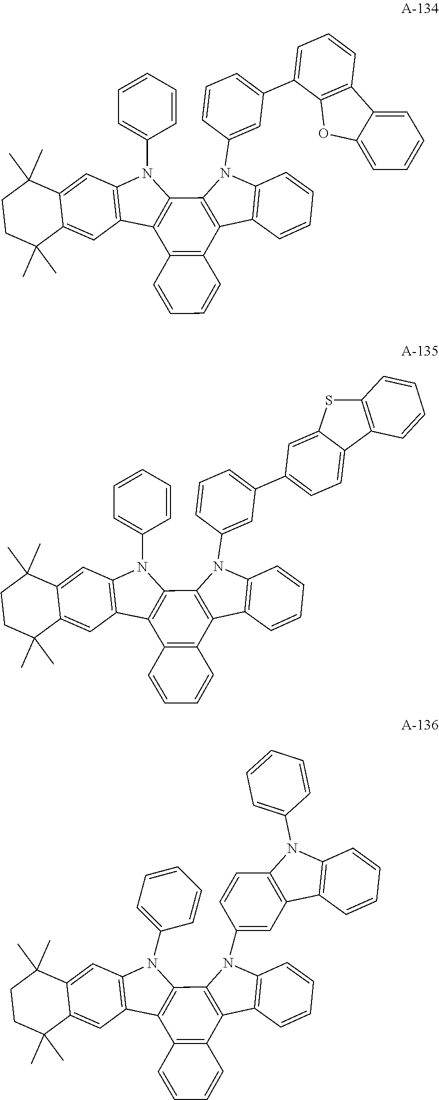 [SMILES: CC1(C)CCC(C)(C)c2cc3c(cc21)c1c2ccccc2c2c4ccccc4n(-c4cccc(-c5cccc6c5oc5ccccc56)c4)c2c1n3-c1ccccc1 ; CC1(C)CCC(C)(C)c2cc3c(cc21)c1c2ccccc2c2c4ccccc4n(-c4cccc(-c5ccc6c(c5)sc5ccccc56)c4)c2c1n3-c1ccccc1 ; CC1(C)CCC(C)(C)c2cc3c(cc21)c1c2ccccc2c2c4ccccc4n(-c4ccc5c(c4)c4ccccc4n5-c4ccccc4)c2c1n3-c1ccccc1]

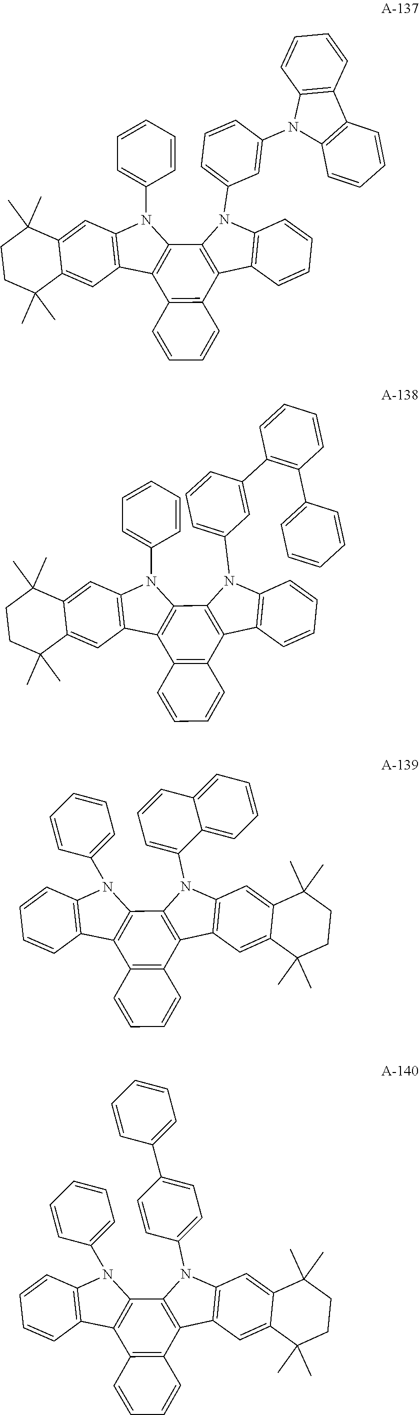 [SMILES: CC1(C)CCC(C)(C)c2cc3c(cc21)c1c2ccccc2c2c4ccccc4n(-c4cccc(-n5c6ccccc6c6ccccc65)c4)c2c1n3-c1ccccc1 ; CC1(C)CCC(C)(C)c2cc3c(cc21)c1c2ccccc2c2c4ccccc4n(-c4cccc(-c5ccccc5-c5ccccc5)c4)c2c1n3-c1ccccc1 ; CC1(C)CCC(C)(C)c2cc3c(cc21)c1c2ccccc2c2c4ccccc4n(-c4ccccc4)c2c1n3-c1cccc2ccccc12 ; CC1(C)CCC(C)(C)c2cc3c(cc21)c1c2ccccc2c2c4ccccc4n(-c4ccccc4)c2c1n3-c1ccc(-c2ccccc2)cc1]

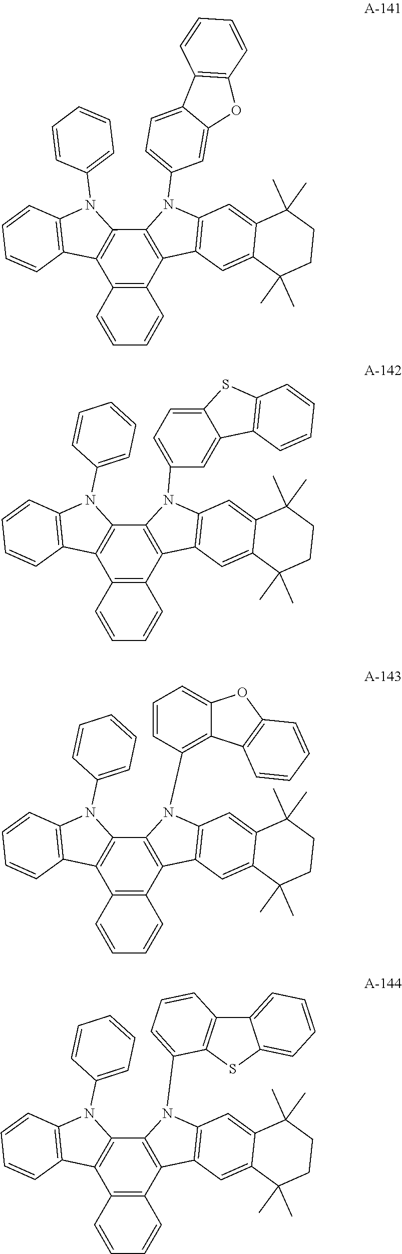 [SMILES: CC1(C)CCC(C)(C)c2cc3c(cc21)c1c2ccccc2c2c4ccccc4n(-c4ccccc4)c2c1n3-c1ccc2c(c1)oc1ccccc12 ; CC1(C)CCC(C)(C)c2cc3c(cc21)c1c2ccccc2c2c4ccccc4n(-c4ccccc4)c2c1n3-c1ccc2sc3ccccc3c2c1 ; CC1(C)CCC(C)(C)c2cc3c(cc21)c1c2ccccc2c2c4ccccc4n(-c4ccccc4)c2c1n3-c1cccc2oc3ccccc3c12 ; CC1(C)CCC(C)(C)c2cc3c(cc21)c1c2ccccc2c2c4ccccc4n(-c4ccccc4)c2c1n3-c1cccc2c1sc1ccccc12]

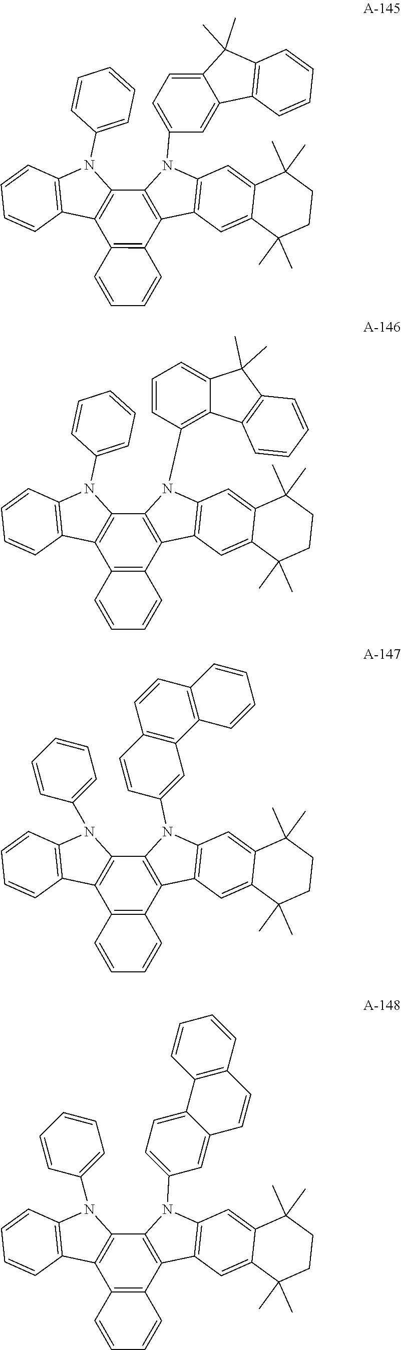 [SMILES: CC1(C)CCC(C)(C)c2cc3c(cc21)c1c2ccccc2c2c4ccccc4n(-c4ccccc4)c2c1n3-c1ccc2c(c1)-c1ccccc1C2(C)C ; CC1(C)CCC(C)(C)c2cc3c(cc21)c1c2ccccc2c2c4ccccc4n(-c4ccccc4)c2c1n3-c1cccc2c1-c1ccccc1C2(C)C ; CC1(C)CCC(C)(C)c2cc3c(cc21)c1c2ccccc2c2c4ccccc4n(-c4ccccc4)c2c1n3-c1ccc2ccc3ccccc3c2c1 ; CC1(C)CCC(C)(C)c2cc3c(cc21)c1c2ccccc2c2c4ccccc4n(-c4ccccc4)c2c1n3-c1ccc2c(ccc3ccccc32)c1]

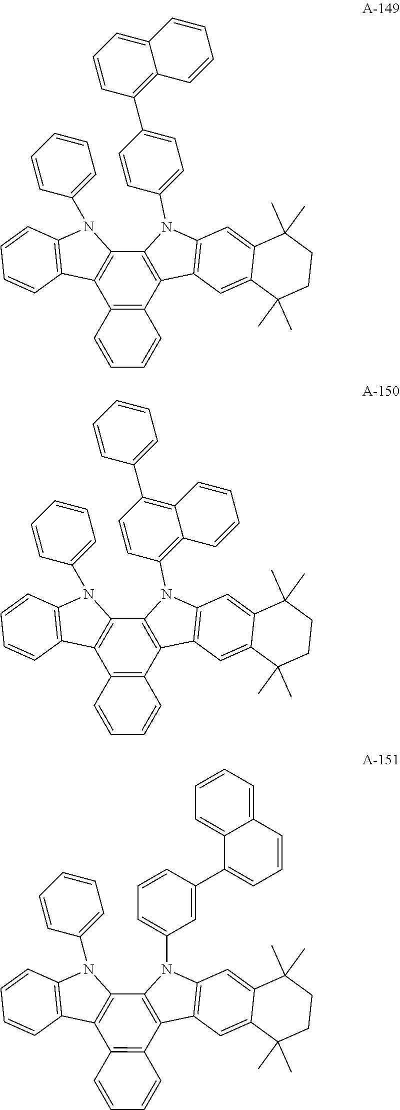

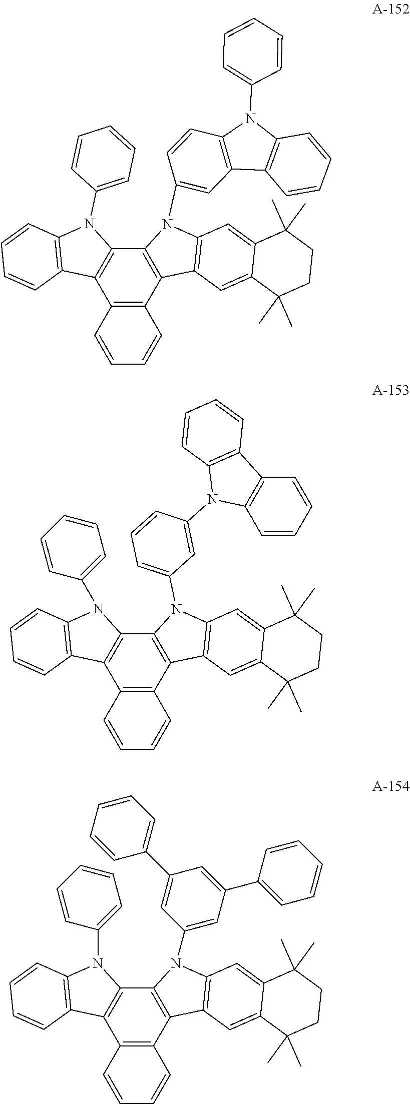 [SMILES: CC1(C)CCC(C)(C)c2cc3c(cc21)c1c2ccccc2c2c4ccccc4n(-c4ccccc4)c2c1n3-c1ccc2c(c1)c1ccccc1n2-c1ccccc1 ; CC1(C)CCC(C)(C)c2cc3c(cc21)c1c2ccccc2c2c4ccccc4n(-c4ccccc4)c2c1n3-c1cccc(-n2c3ccccc3c3ccccc32)c1 ; CC1(C)CCC(C)(C)c2cc3c(cc21)c1c2ccccc2c2c4ccccc4n(-c4ccccc4)c2c1n3-c1cc(-c2ccccc2)cc(-c2ccccc2)c1]

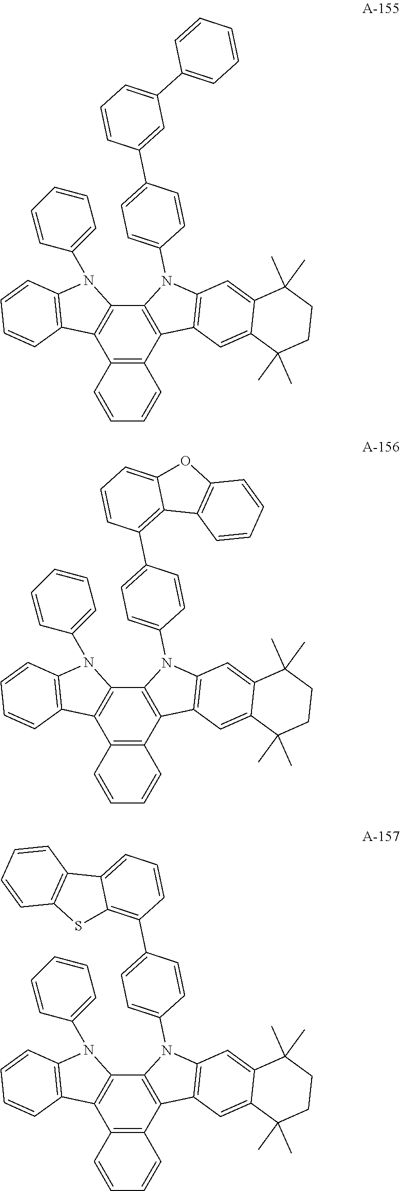 [SMILES: CC1(C)CCC(C)(C)c2cc3c(cc21)c1c2ccccc2c2c4ccccc4n(-c4ccccc4)c2c1n3-c1ccc(-c2cccc(-c3ccccc3)c2)cc1 ; CC1(C)CCC(C)(C)c2cc3c(cc21)c1c2ccccc2c2c4ccccc4n(-c4ccccc4)c2c1n3-c1ccc(-c2cccc3oc4ccccc4c23)cc1 ; CC1(C)CCC(C)(C)c2cc3c(cc21)c1c2ccccc2c2c4ccccc4n(-c4ccccc4)c2c1n3-c1ccc(-c2cccc3c2sc2ccccc23)cc1]

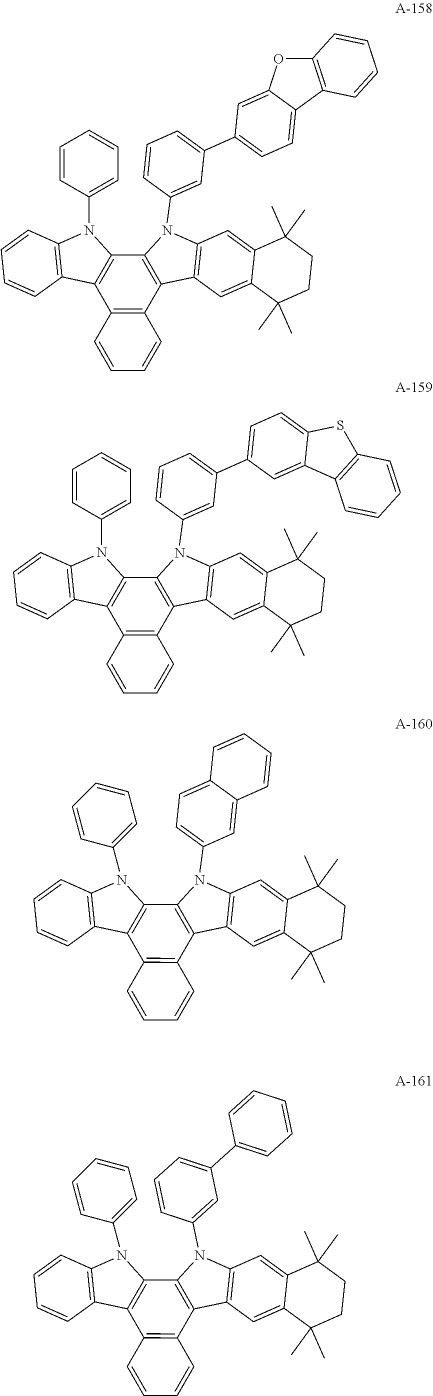 [SMILES: CC1(C)CCC(C)(C)c2cc3c(cc21)c1c2ccccc2c2c4ccccc4n(-c4ccccc4)c2c1n3-c1cccc(-c2ccc3c(c2)oc2ccccc23)c1 ; CC1(C)CCC(C)(C)c2cc3c(cc21)c1c2ccccc2c2c4ccccc4n(-c4ccccc4)c2c1n3-c1cccc(-c2ccc3sc4ccccc4c3c2)c1 ; CC1(C)CCC(C)(C)c2cc3c(cc21)c1c2ccccc2c2c4ccccc4n(-c4ccccc4)c2c1n3-c1ccc2ccccc2c1 ; CC1(C)CCC(C)(C)c2cc3c(cc21)c1c2ccccc2c2c4ccccc4n(-c4ccccc4)c2c1n3-c1cccc(-c2ccccc2)c1]

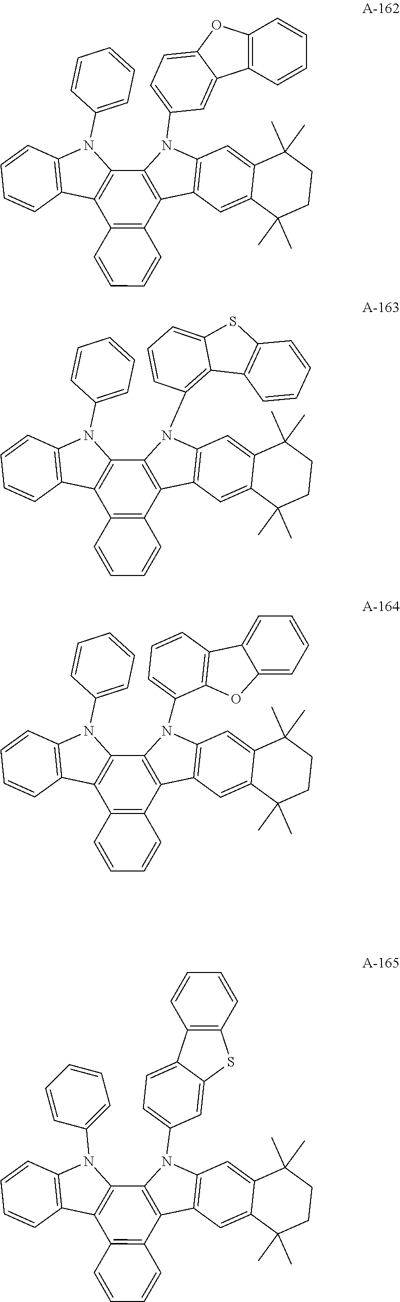 [SMILES: CC1(C)CCC(C)(C)c2cc3c(cc21)c1c2ccccc2c2c4ccccc4n(-c4ccccc4)c2c1n3-c1ccc2oc3ccccc3c2c1 ; CC1(C)CCC(C)(C)c2cc3c(cc21)c1c2ccccc2c2c4ccccc4n(-c4ccccc4)c2c1n3-c1cccc2sc3ccccc3c12 ; CC1(C)CCC(C)(C)c2cc3c(cc21)c1c2ccccc2c2c4ccccc4n(-c4ccccc4)c2c1n3-c1cccc2c1oc1ccccc12 ; CC1(C)CCC(C)(C)c2cc3c(cc21)c1c2ccccc2c2c4ccccc4n(-c4ccccc4)c2c1n3-c1ccc2c(c1)sc1ccccc12]

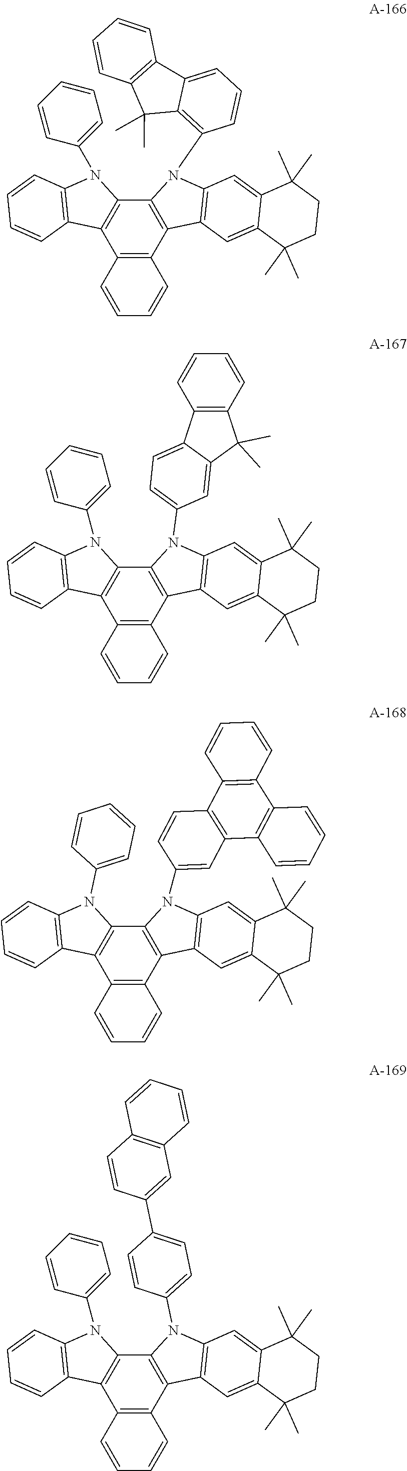

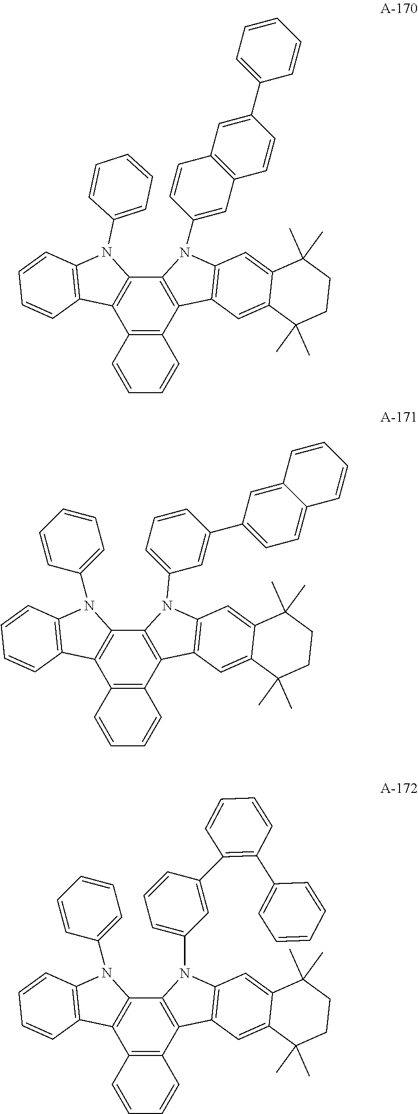 [SMILES: CC1(C)CCC(C)(C)c2cc3c(cc21)c1c2ccccc2c2c4ccccc4n(-c4ccccc4)c2c1n3-c1ccc2cc(-c3ccccc3)ccc2c1 ; CC1(C)CCC(C)(C)c2cc3c(cc21)c1c2ccccc2c2c4ccccc4n(-c4ccccc4)c2c1n3-c1cccc(-c2ccc3ccccc3c2)c1 ; CC1(C)CCC(C)(C)c2cc3c(cc21)c1c2ccccc2c2c4ccccc4n(-c4ccccc4)c2c1n3-c1cccc(-c2ccccc2-c2ccccc2)c1]

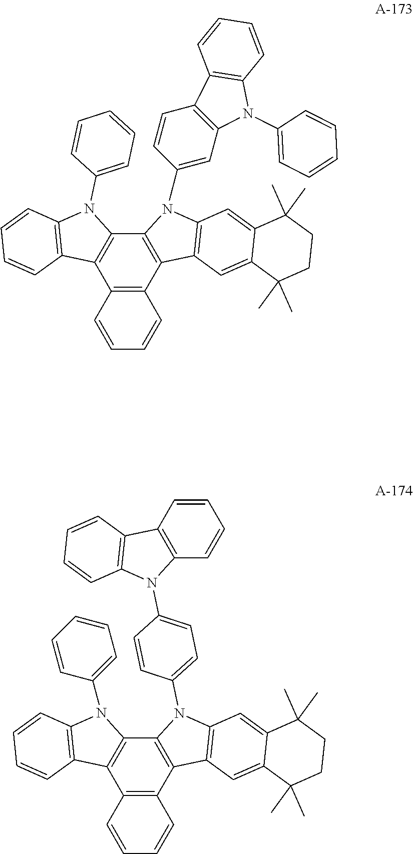 [SMILES: CC1(C)CCC(C)(C)c2cc3c(cc21)c1c2ccccc2c2c4ccccc4n(-c4ccccc4)c2c1n3-c1ccc2c3ccccc3n(-c3ccccc3)c2c1 ; CC1(C)CCC(C)(C)c2cc3c(cc21)c1c2ccccc2c2c4ccccc4n(-c4ccccc4)c2c1n3-c1ccc(-n2c3ccccc3c3ccccc32)cc1]

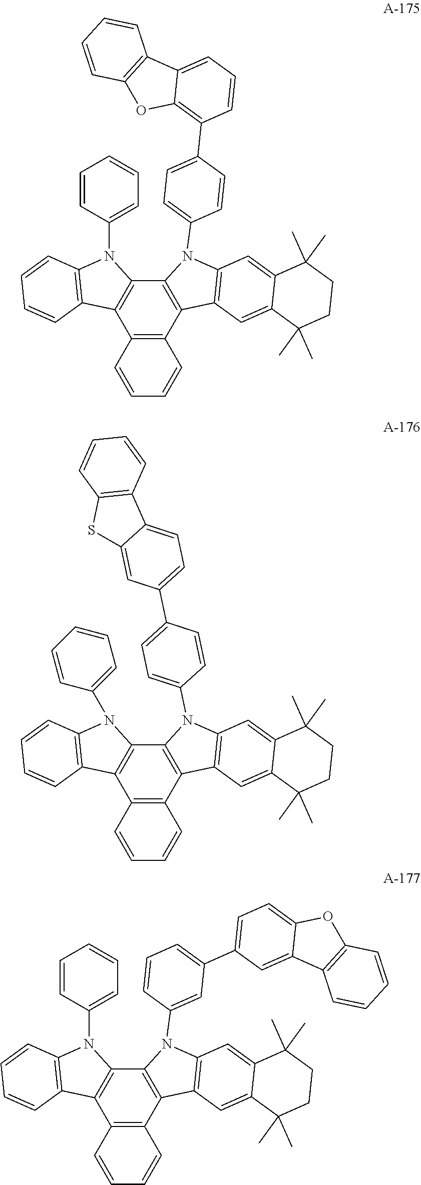

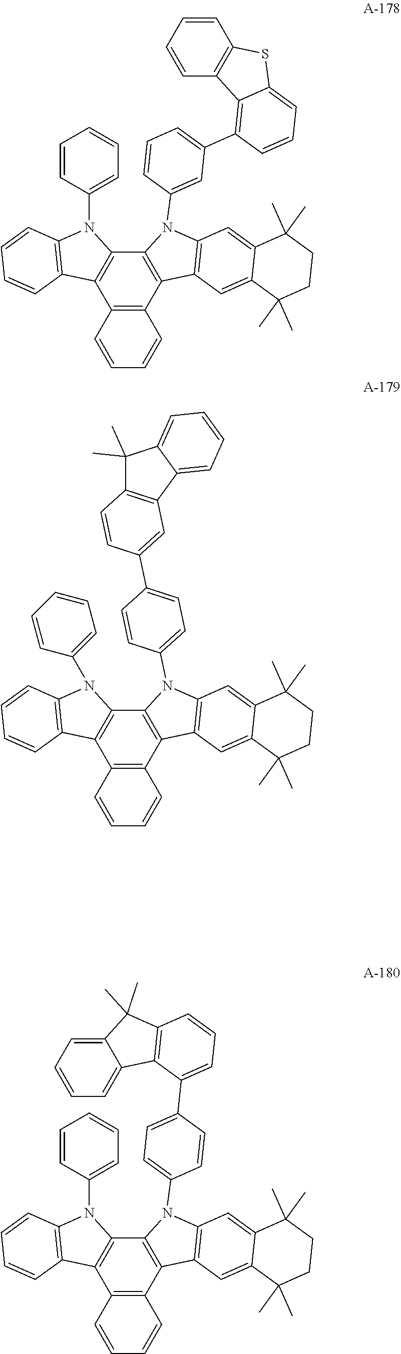 [SMILES: CC1(C)CCC(C)(C)c2cc3c(cc21)c1c2ccccc2c2c4ccccc4n(-c4ccccc4)c2c1n3-c1cccc(-c2cccc3sc4ccccc4c23)c1 ; CC1(C)CCC(C)(C)c2cc3c(cc21)c1c2ccccc2c2c4ccccc4n(-c4ccccc4)c2c1n3-c1ccc(-c2ccc3c(c2)-c2ccccc2C3(C)C)cc1 ; CC1(C)CCC(C)(C)c2cc3c(cc21)c1c2ccccc2c2c4ccccc4n(-c4ccccc4)c2c1n3-c1ccc(-c2cccc3c2-c2ccccc2C3(C)C)cc1]

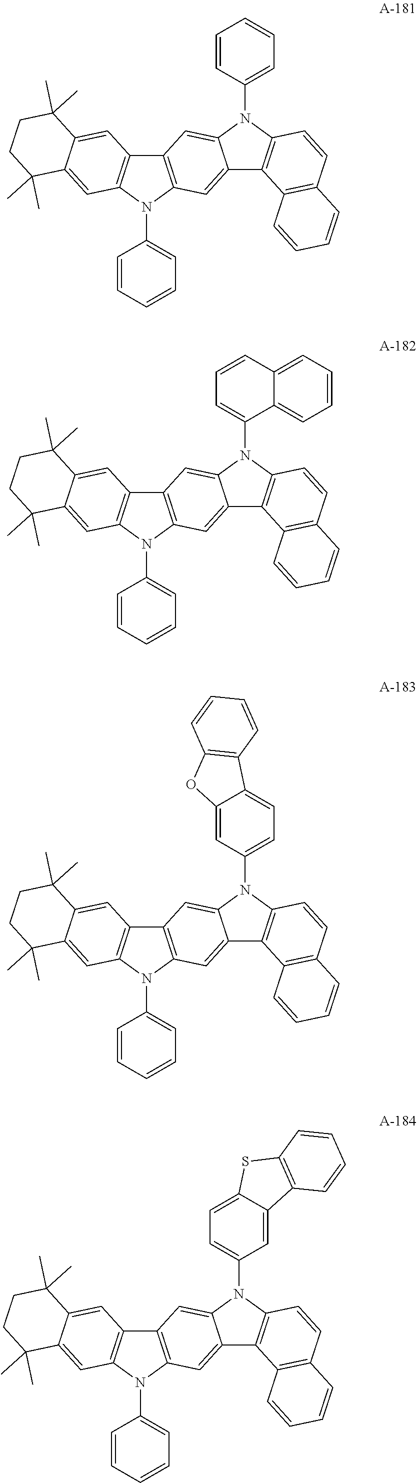 [SMILES: CC1(C)CCC(C)(C)c2cc3c(cc21)c1cc2c(cc1n3-c1ccccc1)c1c3ccccc3ccc1n2-c1ccccc1 ; CC1(C)CCC(C)(C)c2cc3c(cc21)c1cc2c(cc1n3-c1ccccc1)c1c3ccccc3ccc1n2-c1cccc2ccccc12 ; CC1(C)CCC(C)(C)c2cc3c(cc21)c1cc2c(cc1n3-c1ccccc1)c1c3ccccc3ccc1n2-c1ccc2c(c1)oc1ccccc12 ; CC1(C)CCC(C)(C)c2cc3c(cc21)c1cc2c(cc1n3-c1ccccc1)c1c3ccccc3ccc1n2-c1ccc2sc3ccccc3c2c1]

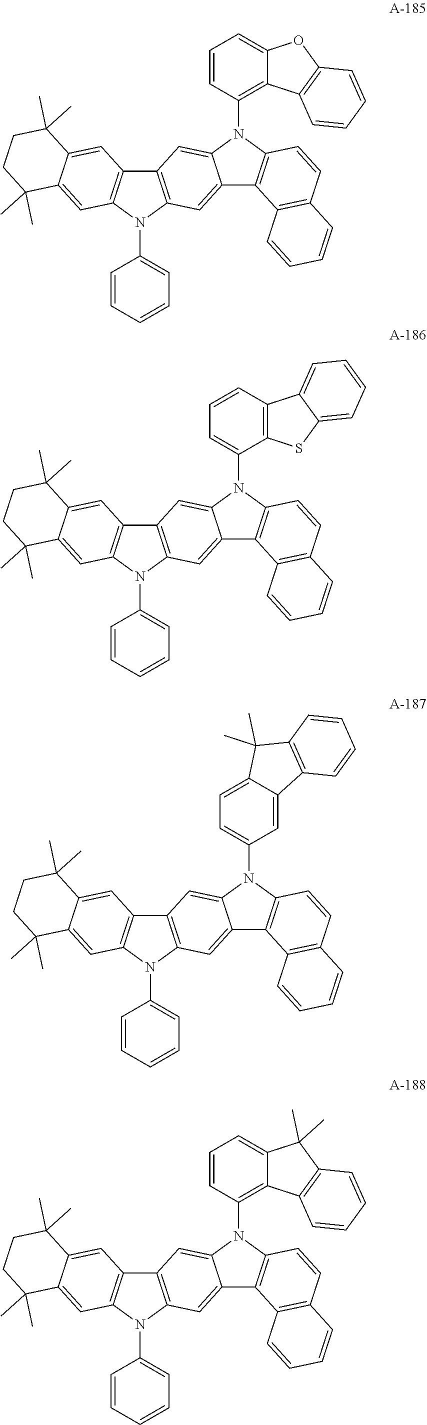 [SMILES: CC1(C)CCC(C)(C)c2cc3c(cc21)c1cc2c(cc1n3-c1ccccc1)c1c3ccccc3ccc1n2-c1cccc2oc3ccccc3c12 ; CC1(C)CCC(C)(C)c2cc3c(cc21)c1cc2c(cc1n3-c1ccccc1)c1c3ccccc3ccc1n2-c1cccc2c1sc1ccccc12 ; CC1(C)CCC(C)(C)c2cc3c(cc21)c1cc2c(cc1n3-c1ccccc1)c1c3ccccc3ccc1n2-c1ccc2c(c1)-c1ccccc1C2(C)C ; CC1(C)CCC(C)(C)c2cc3c(cc21)c1cc2c(cc1n3-c1ccccc1)c1c3ccccc3ccc1n2-c1cccc2c1-c1ccccc1C2(C)C]

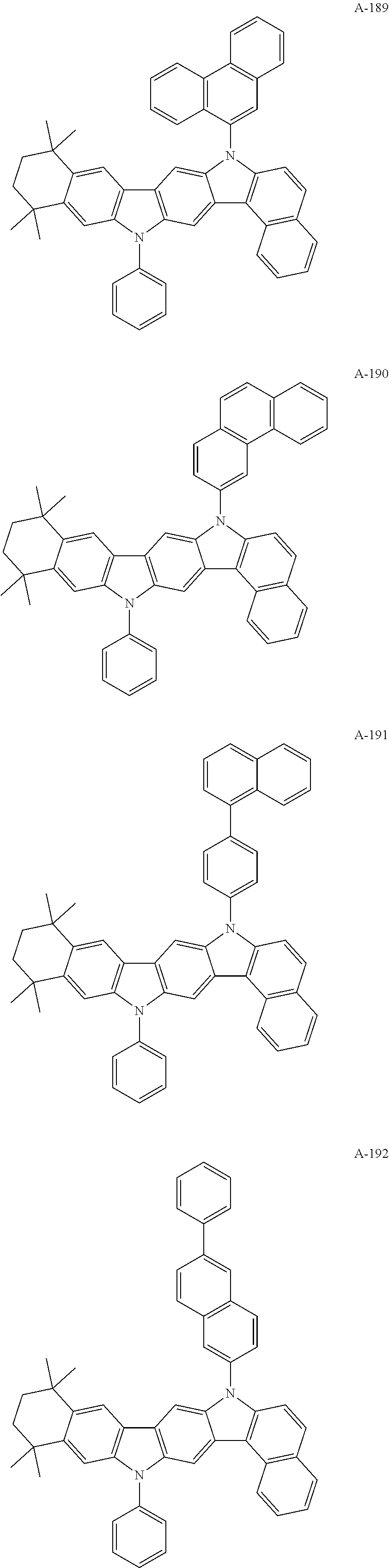 [SMILES: CC1(C)CCC(C)(C)c2cc3c(cc21)c1cc2c(cc1n3-c1ccccc1)c1c3ccccc3ccc1n2-c1cc2ccccc2c2ccccc12 ; CC1(C)CCC(C)(C)c2cc3c(cc21)c1cc2c(cc1n3-c1ccccc1)c1c3ccccc3ccc1n2-c1ccc2ccc3ccccc3c2c1 ; CC1(C)CCC(C)(C)c2cc3c(cc21)c1cc2c(cc1n3-c1ccccc1)c1c3ccccc3ccc1n2-c1ccc(-c2cccc3ccccc23)cc1 ; CC1(C)CCC(C)(C)c2cc3c(cc21)c1cc2c(cc1n3-c1ccccc1)c1c3ccccc3ccc1n2-c1ccc2cc(-c3ccccc3)ccc2c1]

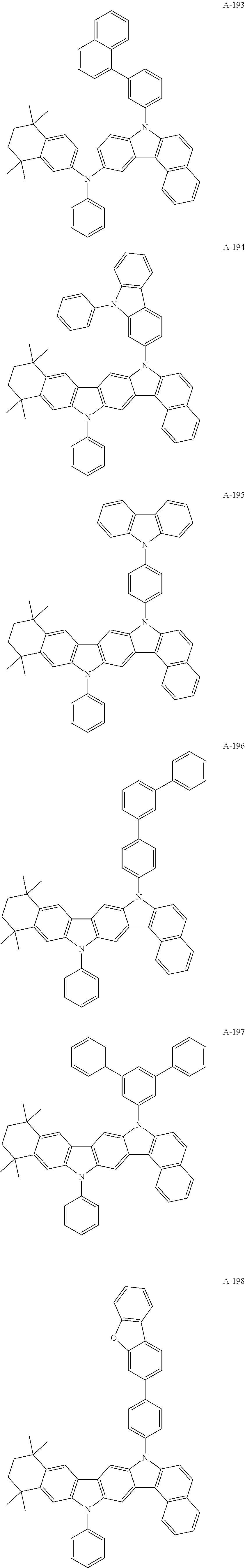

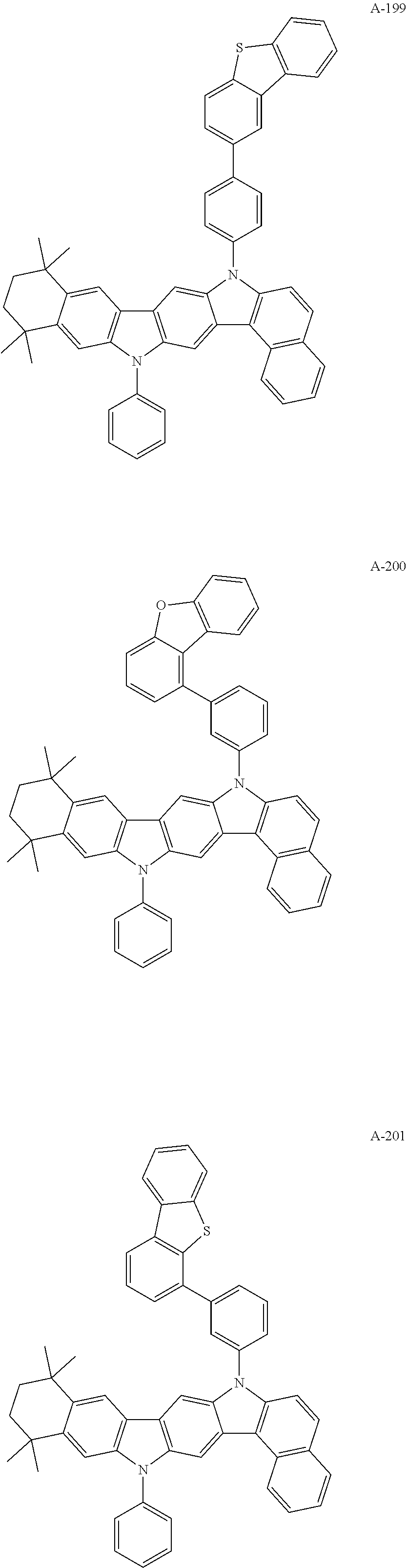 [SMILES: CC1(C)CCC(C)(C)c2cc3c(cc21)c1cc2c(cc1n3-c1ccccc1)c1c3ccccc3ccc1n2-c1ccc(-c2ccc3sc4ccccc4c3c2)cc1 ; CC1(C)CCC(C)(C)c2cc3c(cc21)c1cc2c(cc1n3-c1ccccc1)c1c3ccccc3ccc1n2-c1cccc(-c2cccc3oc4ccccc4c23)c1 ; CC1(C)CCC(C)(C)c2cc3c(cc21)c1cc2c(cc1n3-c1ccccc1)c1c3ccccc3ccc1n2-c1cccc(-c2cccc3c2sc2ccccc23)c1]

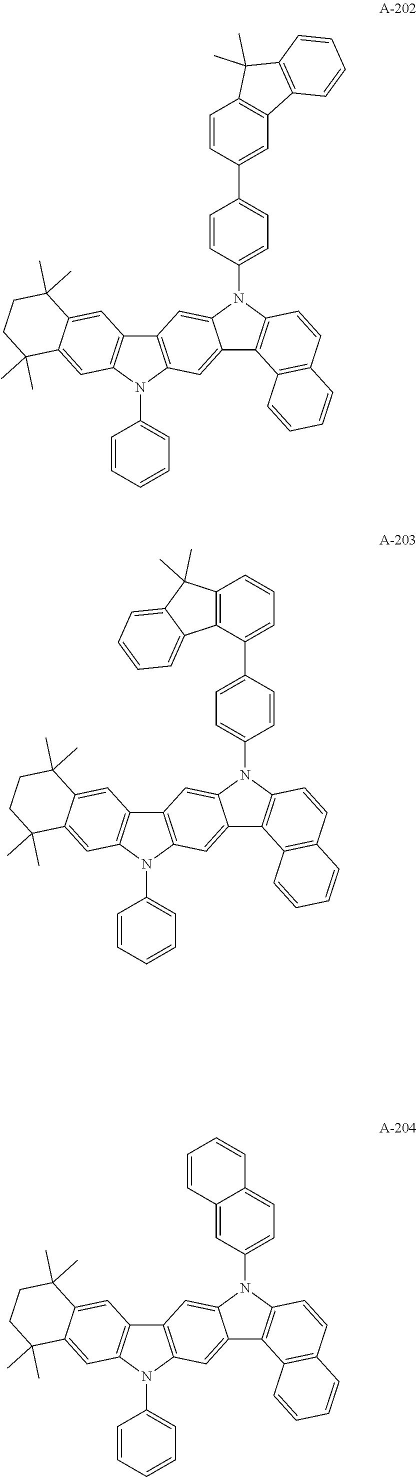 [SMILES: CC1(C)CCC(C)(C)c2cc3c(cc21)c1cc2c(cc1n3-c1ccccc1)c1c3ccccc3ccc1n2-c1ccc(-c2ccc3c(c2)-c2ccccc2C3(C)C)cc1 ; CC1(C)CCC(C)(C)c2cc3c(cc21)c1cc2c(cc1n3-c1ccccc1)c1c3ccccc3ccc1n2-c1ccc(-c2cccc3c2-c2ccccc2C3(C)C)cc1 ; CC1(C)CCC(C)(C)c2cc3c(cc21)c1cc2c(cc1n3-c1ccccc1)c1c3ccccc3ccc1n2-c1ccc2ccccc2c1]

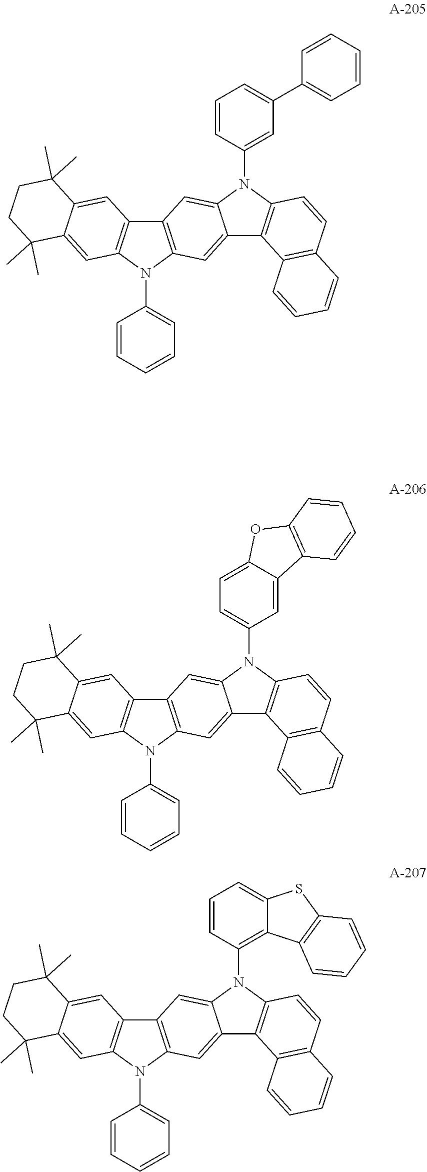 [SMILES: CC1(C)CCC(C)(C)c2cc3c(cc21)c1cc2c(cc1n3-c1ccccc1)c1c3ccccc3ccc1n2-c1cccc(-c2ccccc2)c1 ; CC1(C)CCC(C)(C)c2cc3c(cc21)c1cc2c(cc1n3-c1ccccc1)c1c3ccccc3ccc1n2-c1ccc2oc3ccccc3c2c1 ; CC1(C)CCC(C)(C)c2cc3c(cc21)c1cc2c(cc1n3-c1ccccc1)c1c3ccccc3ccc1n2-c1cccc2sc3ccccc3c12]

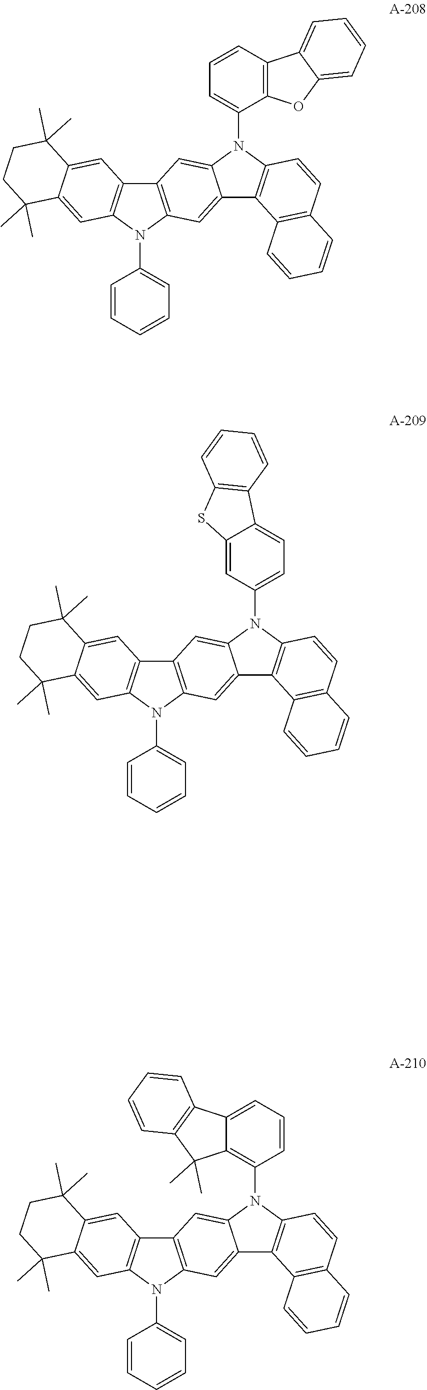 [SMILES: CC1(C)CCC(C)(C)c2cc3c(cc21)c1cc2c(cc1n3-c1ccccc1)c1c3ccccc3ccc1n2-c1cccc2c1oc1ccccc12 ; CC1(C)CCC(C)(C)c2cc3c(cc21)c1cc2c(cc1n3-c1ccccc1)c1c3ccccc3ccc1n2-c1ccc2c(c1)sc1ccccc12 ; CC1(C)CCC(C)(C)c2cc3c(cc21)c1cc2c(cc1n3-c1ccccc1)c1c3ccccc3ccc1n2-c1cccc2c1C(C)(C)c1ccccc1-2]

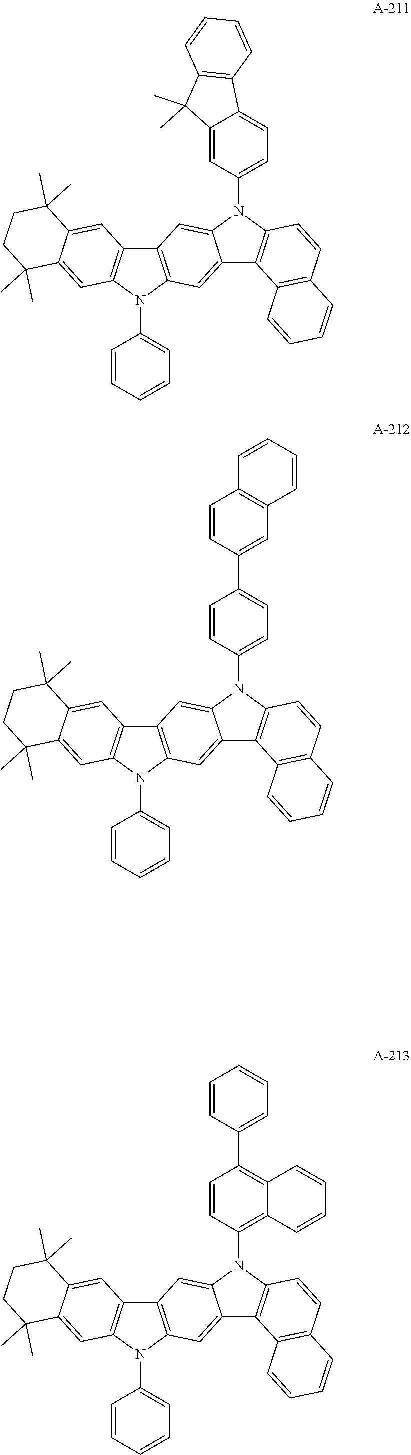 [SMILES: CC1(C)CCC(C)(C)c2cc3c(cc21)c1cc2c(cc1n3-c1ccccc1)c1c3ccccc3ccc1n2-c1ccc2c(c1)C(C)(C)c1ccccc1-2 ; CC1(C)CCC(C)(C)c2cc3c(cc21)c1cc2c(cc1n3-c1ccccc1)c1c3ccccc3ccc1n2-c1ccc(-c2ccc3ccccc3c2)cc1 ; CC1(C)CCC(C)(C)c2cc3c(cc21)c1cc2c(cc1n3-c1ccccc1)c1c3ccccc3ccc1n2-c1ccc(-c2ccccc2)c2ccccc12]

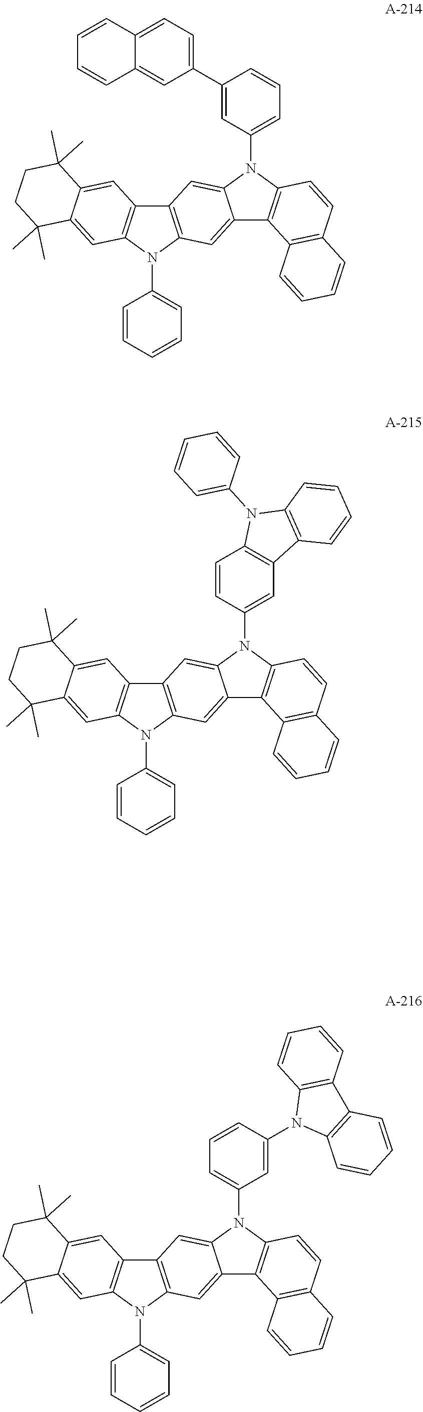 [SMILES: CC1(C)CCC(C)(C)c2cc3c(cc21)c1cc2c(cc1n3-c1ccccc1)c1c3ccccc3ccc1n2-c1cccc(-c2ccc3ccccc3c2)c1 ; CC1(C)CCC(C)(C)c2cc3c(cc21)c1cc2c(cc1n3-c1ccccc1)c1c3ccccc3ccc1n2-c1ccc2c(c1)c1ccccc1n2-c1ccccc1 ; CC1(C)CCC(C)(C)c2cc3c(cc21)c1cc2c(cc1n3-c1ccccc1)c1c3ccccc3ccc1n2-c1cccc(-n2c3ccccc3c3ccccc32)c1]

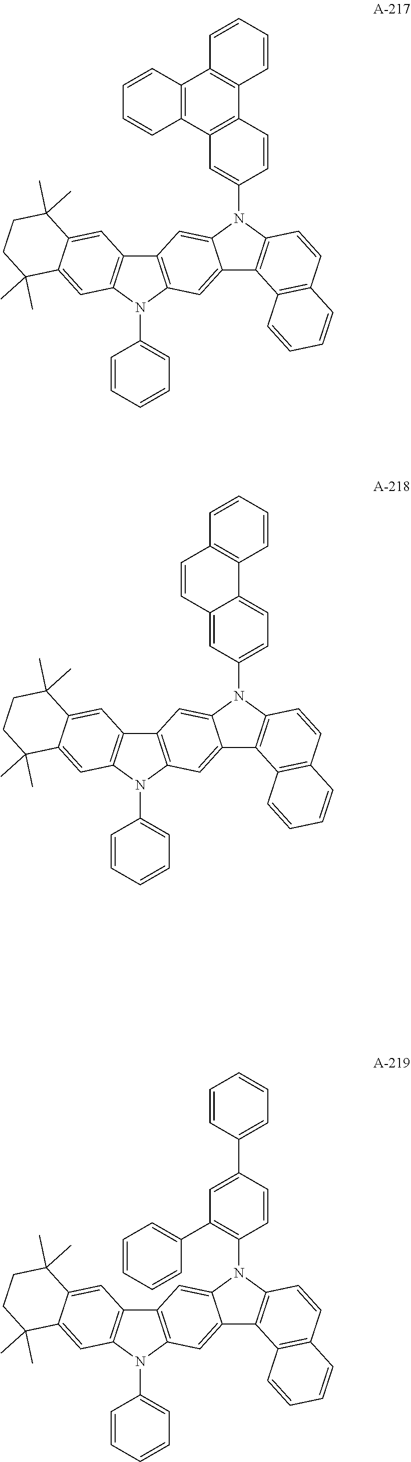 [SMILES: CC1(C)CCC(C)(C)c2cc3c(cc21)c1cc2c(cc1n3-c1ccccc1)c1c3ccccc3ccc1n2-c1ccc2c3ccccc3c3ccccc3c2c1 ; CC1(C)CCC(C)(C)c2cc3c(cc21)c1cc2c(cc1n3-c1ccccc1)c1c3ccccc3ccc1n2-c1ccc2c(ccc3ccccc32)c1 ; CC1(C)CCC(C)(C)c2cc3c(cc21)c1cc2c(cc1n3-c1ccccc1)c1c3ccccc3ccc1n2-c1ccc(-c2ccccc2)cc1-c1ccccc1]

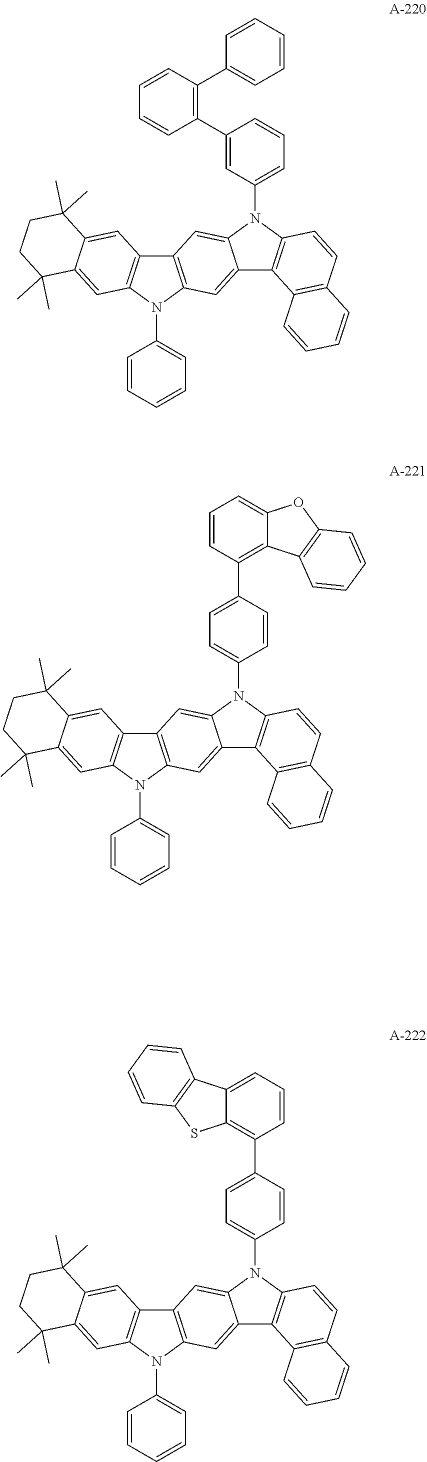 [SMILES: CC1(C)CCC(C)(C)c2cc3c(cc21)c1cc2c(cc1n3-c1ccccc1)c1c3ccccc3ccc1n2-c1cccc(-c2ccccc2-c2ccccc2)c1 ; CC1(C)CCC(C)(C)c2cc3c(cc21)c1cc2c(cc1n3-c1ccccc1)c1c3ccccc3ccc1n2-c1ccc(-c2cccc3oc4ccccc4c23)cc1 ; CC1(C)CCC(C)(C)c2cc3c(cc21)c1cc2c(cc1n3-c1ccccc1)c1c3ccccc3ccc1n2-c1ccc(-c2cccc3c2sc2ccccc23)cc1]

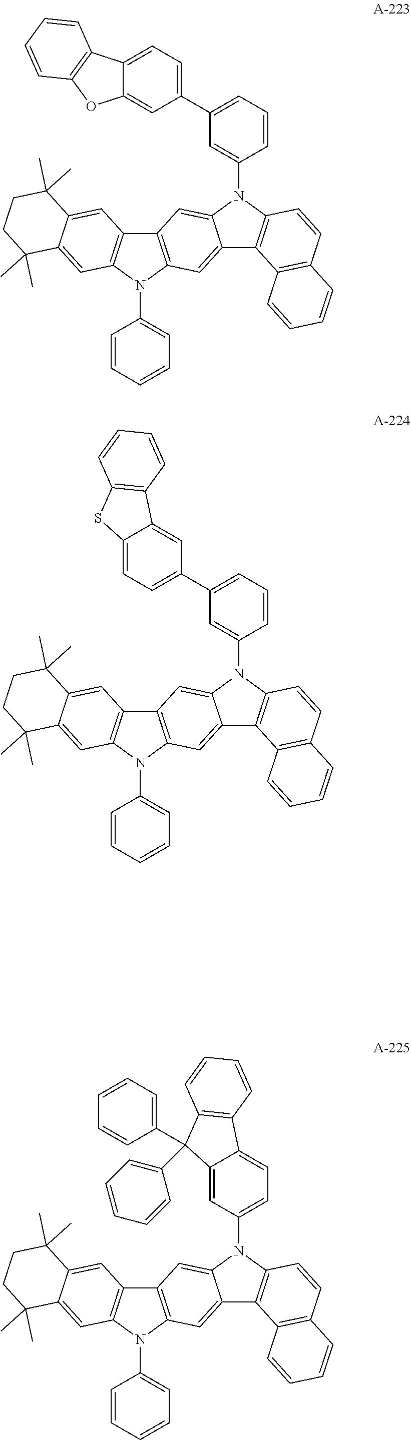 [SMILES: CC1(C)CCC(C)(C)c2cc3c(cc21)c1cc2c(cc1n3-c1ccccc1)c1c3ccccc3ccc1n2-c1cccc(-c2ccc3c(c2)oc2ccccc23)c1 ; CC1(C)CCC(C)(C)c2cc3c(cc21)c1cc2c(cc1n3-c1ccccc1)c1c3ccccc3ccc1n2-c1cccc(-c2ccc3sc4ccccc4c3c2)c1 ; CC1(C)CCC(C)(C)c2cc3c(cc21)c1cc2c(cc1n3-c1ccccc1)c1c3ccccc3ccc1n2-c1ccc2c(c1)C(c1ccccc1)(c1ccccc1)c1ccccc1-2]

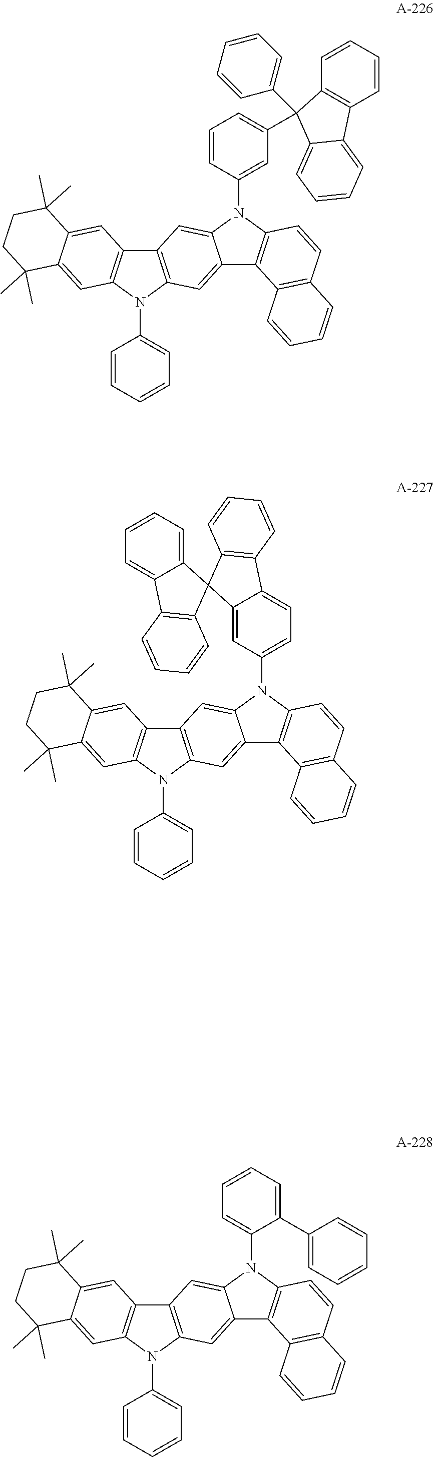 [SMILES: CC1(C)CCC(C)(C)c2cc3c(cc21)c1cc2c(cc1n3-c1ccccc1)c1c3ccccc3ccc1n2-c1cccc(C2(c3ccccc3)c3ccccc3-c3ccccc32)c1 ; CC1(C)CCC(C)(C)c2cc3c(cc21)c1cc2c(cc1n3-c1ccccc1)c1c3ccccc3ccc1n2-c1ccc2c(c1)C1(c3ccccc3-c3ccccc31)c1ccccc1-2 ; CC1(C)CCC(C)(C)c2cc3c(cc21)c1cc2c(cc1n3-c1ccccc1)c1c3ccccc3ccc1n2-c1ccccc1-c1ccccc1]

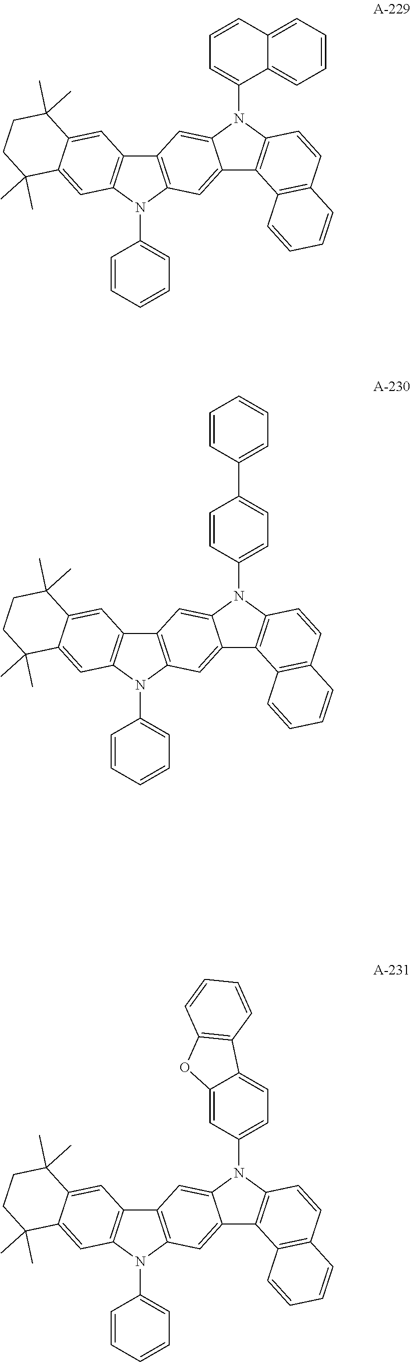 [SMILES: CC1(C)CCC(C)(C)c2cc3c(cc21)c1cc2c(cc1n3-c1ccccc1)c1c3ccccc3ccc1n2-c1cccc2ccccc12 ; CC1(C)CCC(C)(C)c2cc3c(cc21)c1cc2c(cc1n3-c1ccccc1)c1c3ccccc3ccc1n2-c1ccc(-c2ccccc2)cc1 ; CC1(C)CCC(C)(C)c2cc3c(cc21)c1cc2c(cc1n3-c1ccccc1)c1c3ccccc3ccc1n2-c1ccc2c(c1)oc1ccccc12]

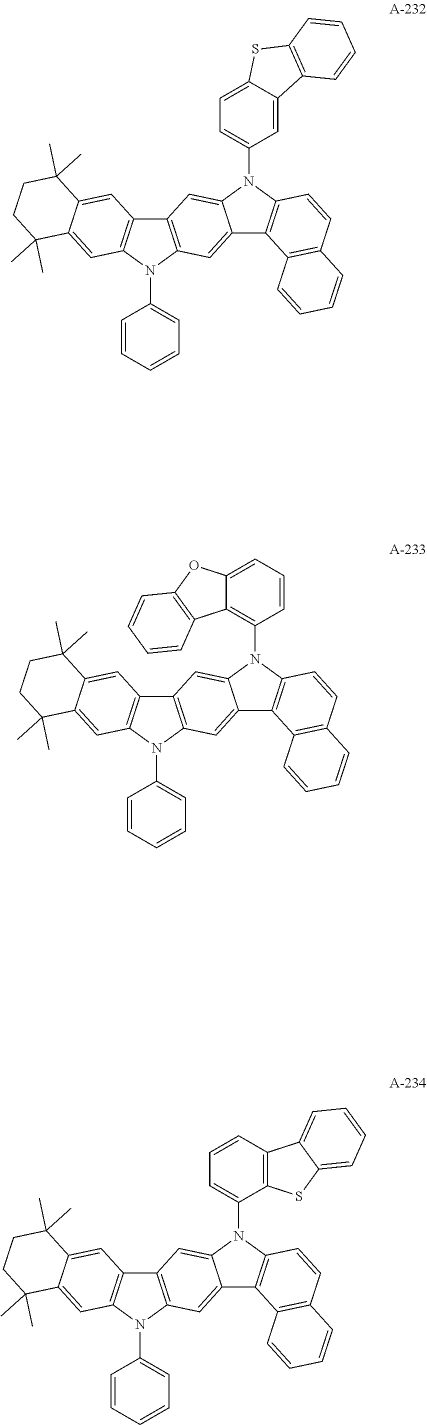 [SMILES: CC1(C)CCC(C)(C)c2cc3c(cc21)c1cc2c(cc1n3-c1ccccc1)c1c3ccccc3ccc1n2-c1ccc2sc3ccccc3c2c1 ; CC1(C)CCC(C)(C)c2cc3c(cc21)c1cc2c(cc1n3-c1ccccc1)c1c3ccccc3ccc1n2-c1cccc2oc3ccccc3c12 ; CC1(C)CCC(C)(C)c2cc3c(cc21)c1cc2c(cc1n3-c1ccccc1)c1c3ccccc3ccc1n2-c1cccc2c1sc1ccccc12]

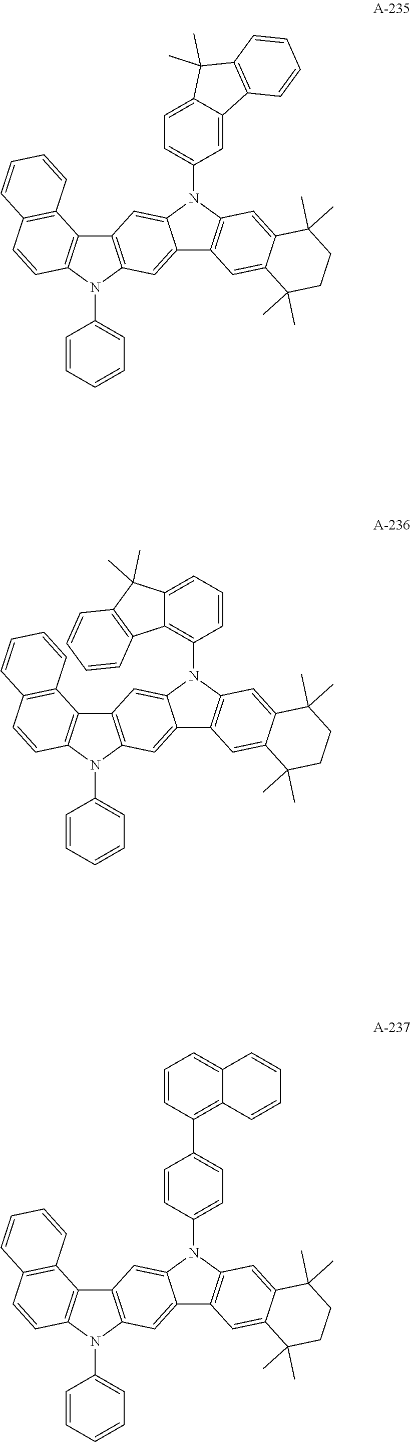 [SMILES: CC1(C)CCC(C)(C)c2cc3c(cc21)c1cc2c(cc1n3-c1ccc3c(c1)-c1ccccc1C3(C)C)c1c3ccccc3ccc1n2-c1ccccc1 ; CC1(C)CCC(C)(C)c2cc3c(cc21)c1cc2c(cc1n3-c1cccc3c1-c1ccccc1C3(C)C)c1c3ccccc3ccc1n2-c1ccccc1 ; CC1(C)CCC(C)(C)c2cc3c(cc21)c1cc2c(cc1n3-c1ccc(-c3cccc4ccccc34)cc1)c1c3ccccc3ccc1n2-c1ccccc1]

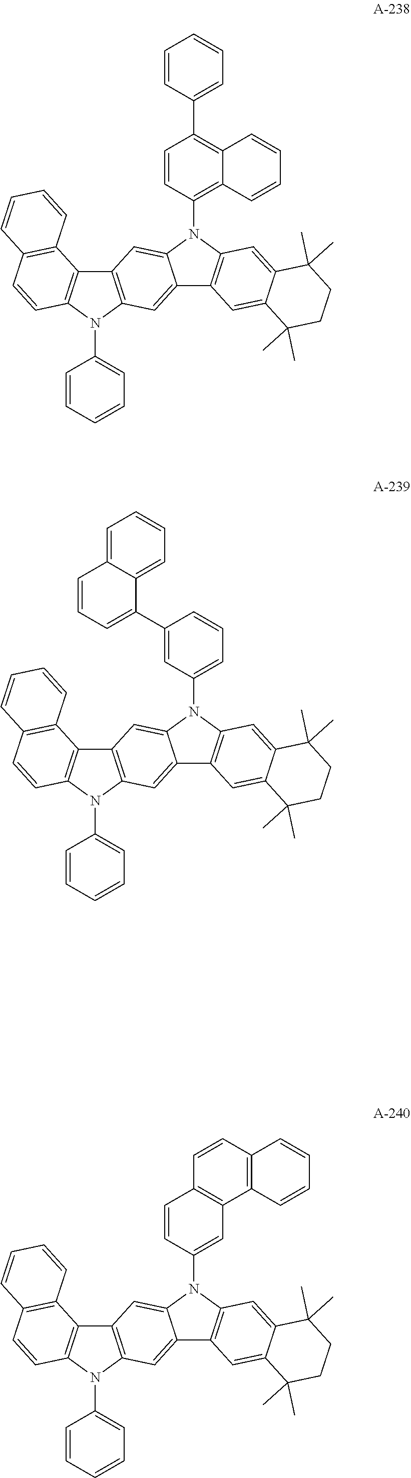 [SMILES: CC1(C)CCC(C)(C)c2cc3c(cc21)c1cc2c(cc1n3-c1ccc(-c3ccccc3)c3ccccc13)c1c3ccccc3ccc1n2-c1ccccc1 ; CC1(C)CCC(C)(C)c2cc3c(cc21)c1cc2c(cc1n3-c1cccc(-c3cccc4ccccc34)c1)c1c3ccccc3ccc1n2-c1ccccc1 ; CC1(C)CCC(C)(C)c2cc3c(cc21)c1cc2c(cc1n3-c1ccc3ccc4ccccc4c3c1)c1c3ccccc3ccc1n2-c1ccccc1]

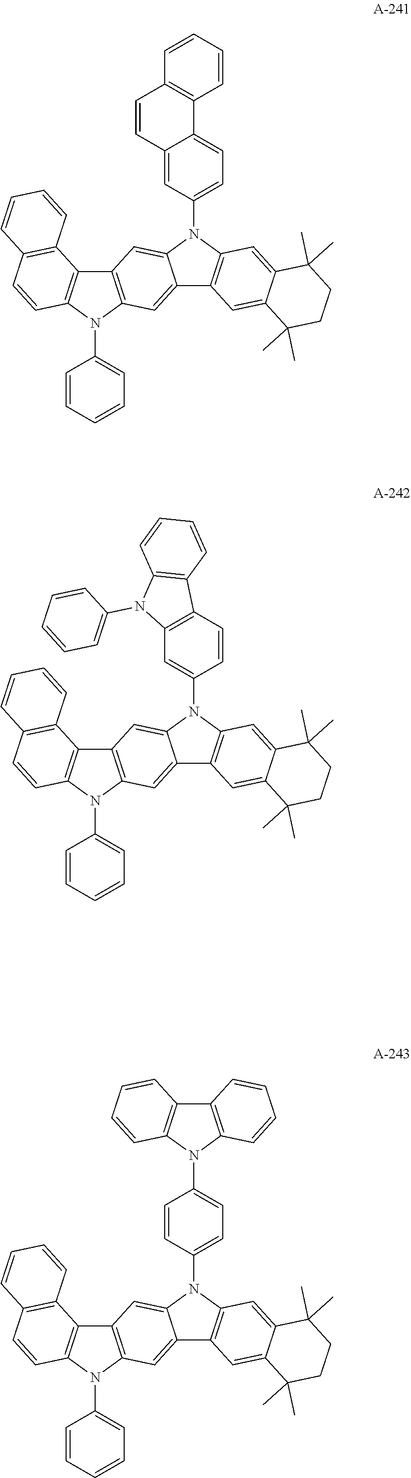 [SMILES: CC1(C)CCC(C)(C)c2cc3c(cc21)c1cc2c(cc1n3-c1ccc3c(ccc4ccccc43)c1)c1c3ccccc3ccc1n2-c1ccccc1 ; CC1(C)CCC(C)(C)c2cc3c(cc21)c1cc2c(cc1n3-c1ccc3c4ccccc4n(-c4ccccc4)c3c1)c1c3ccccc3ccc1n2-c1ccccc1 ; CC1(C)CCC(C)(C)c2cc3c(cc21)c1cc2c(cc1n3-c1ccc(-n3c4ccccc4c4ccccc43)cc1)c1c3ccccc3ccc1n2-c1ccccc1]

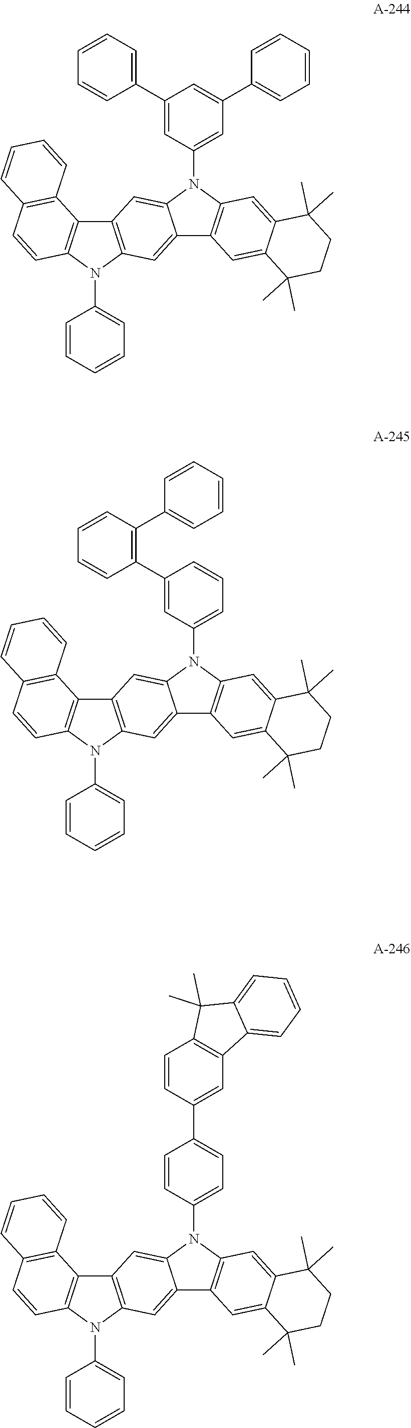 [SMILES: CC1(C)CCC(C)(C)c2cc3c(cc21)c1cc2c(cc1n3-c1cc(-c3ccccc3)cc(-c3ccccc3)c1)c1c3ccccc3ccc1n2-c1ccccc1 ; CC1(C)CCC(C)(C)c2cc3c(cc21)c1cc2c(cc1n3-c1cccc(-c3ccccc3-c3ccccc3)c1)c1c3ccccc3ccc1n2-c1ccccc1 ; CC1(C)CCC(C)(C)c2cc3c(cc21)c1cc2c(cc1n3-c1ccc(-c3ccc4c(c3)-c3ccccc3C4(C)C)cc1)c1c3ccccc3ccc1n2-c1ccccc1]

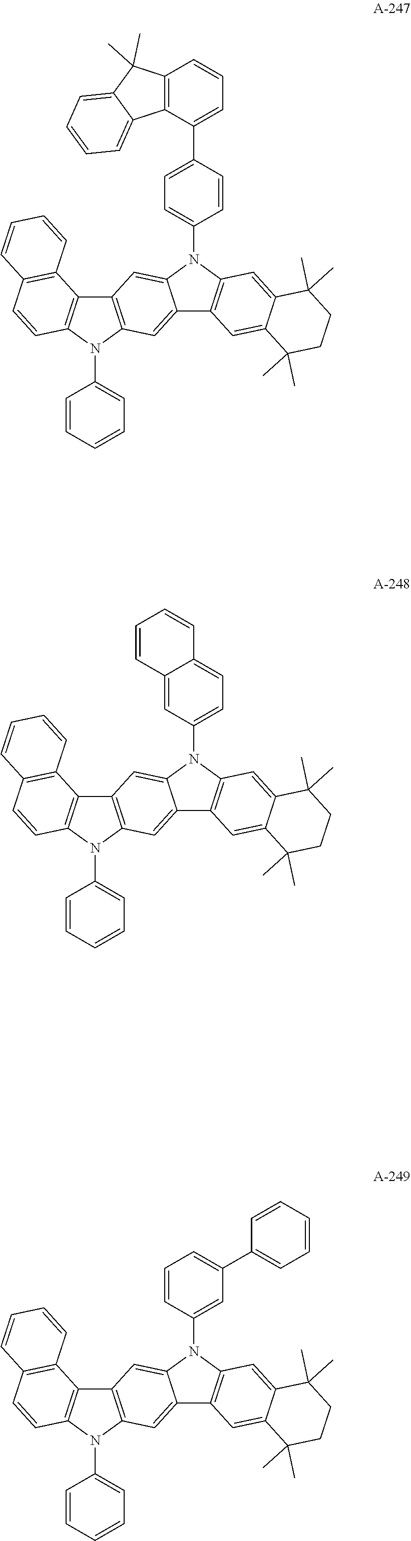 [SMILES: CC1(C)CCC(C)(C)c2cc3c(cc21)c1cc2c(cc1n3-c1ccc(-c3cccc4c3-c3ccccc3C4(C)C)cc1)c1c3ccccc3ccc1n2-c1ccccc1 ; CC1(C)CCC(C)(C)c2cc3c(cc21)c1cc2c(cc1n3-c1ccc3ccccc3c1)c1c3ccccc3ccc1n2-c1ccccc1 ; CC1(C)CCC(C)(C)c2cc3c(cc21)c1cc2c(cc1n3-c1cccc(-c3ccccc3)c1)c1c3ccccc3ccc1n2-c1ccccc1]

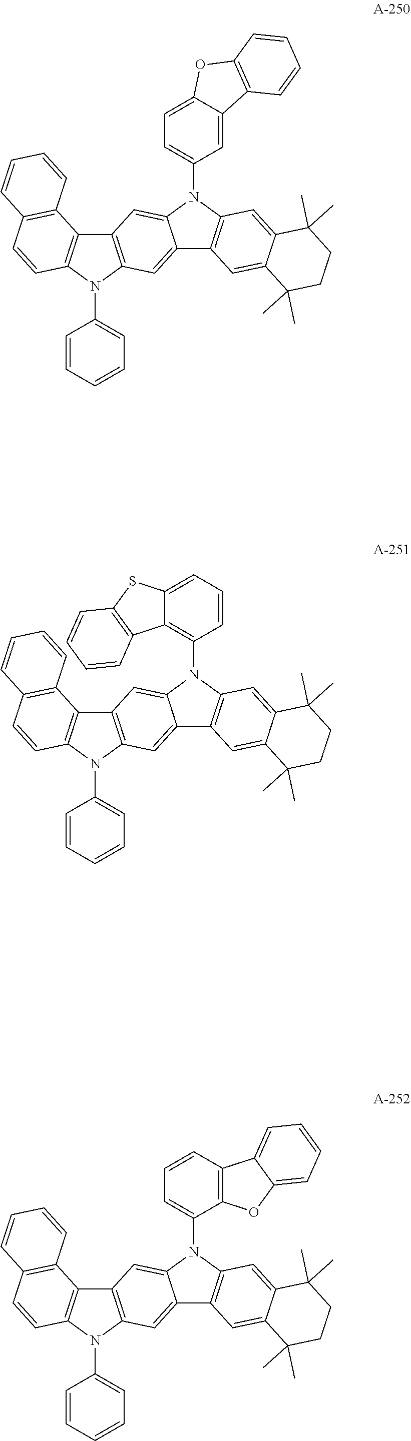 [SMILES: CC1(C)CCC(C)(C)c2cc3c(cc21)c1cc2c(cc1n3-c1ccc3oc4ccccc4c3c1)c1c3ccccc3ccc1n2-c1ccccc1 ; CC1(C)CCC(C)(C)c2cc3c(cc21)c1cc2c(cc1n3-c1cccc3sc4ccccc4c13)c1c3ccccc3ccc1n2-c1ccccc1 ; CC1(C)CCC(C)(C)c2cc3c(cc21)c1cc2c(cc1n3-c1cccc3c1oc1ccccc13)c1c3ccccc3ccc1n2-c1ccccc1]

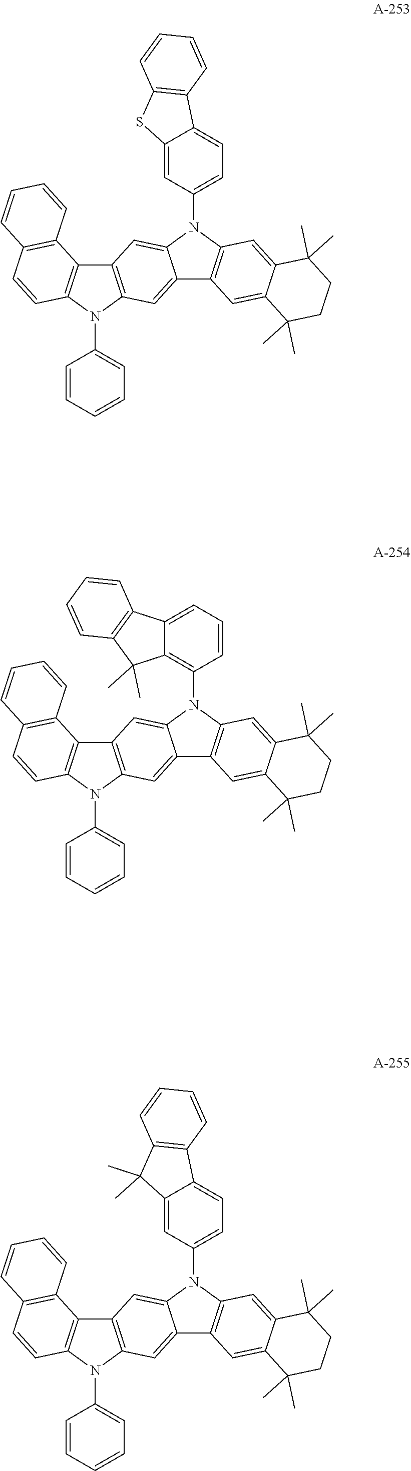 [SMILES: CC1(C)CCC(C)(C)c2cc3c(cc21)c1cc2c(cc1n3-c1ccc3c(c1)sc1ccccc13)c1c3ccccc3ccc1n2-c1ccccc1 ; CC1(C)CCC(C)(C)c2cc3c(cc21)c1cc2c(cc1n3-c1cccc3c1C(C)(C)c1ccccc1-3)c1c3ccccc3ccc1n2-c1ccccc1 ; CC1(C)CCC(C)(C)c2cc3c(cc21)c1cc2c(cc1n3-c1ccc3c(c1)C(C)(C)c1ccccc1-3)c1c3ccccc3ccc1n2-c1ccccc1]

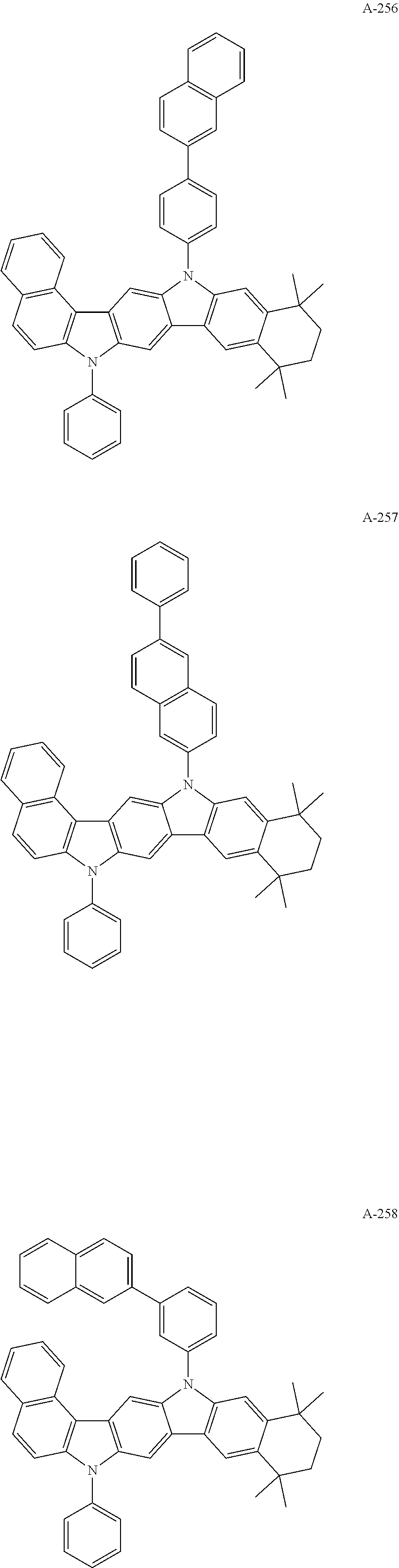 [SMILES: CC1(C)CCC(C)(C)c2cc3c(cc21)c1cc2c(cc1n3-c1ccc(-c3ccc4ccccc4c3)cc1)c1c3ccccc3ccc1n2-c1ccccc1 ; CC1(C)CCC(C)(C)c2cc3c(cc21)c1cc2c(cc1n3-c1ccc3cc(-c4ccccc4)ccc3c1)c1c3ccccc3ccc1n2-c1ccccc1 ; CC1(C)CCC(C)(C)c2cc3c(cc21)c1cc2c(cc1n3-c1cccc(-c3ccc4ccccc4c3)c1)c1c3ccccc3ccc1n2-c1ccccc1]

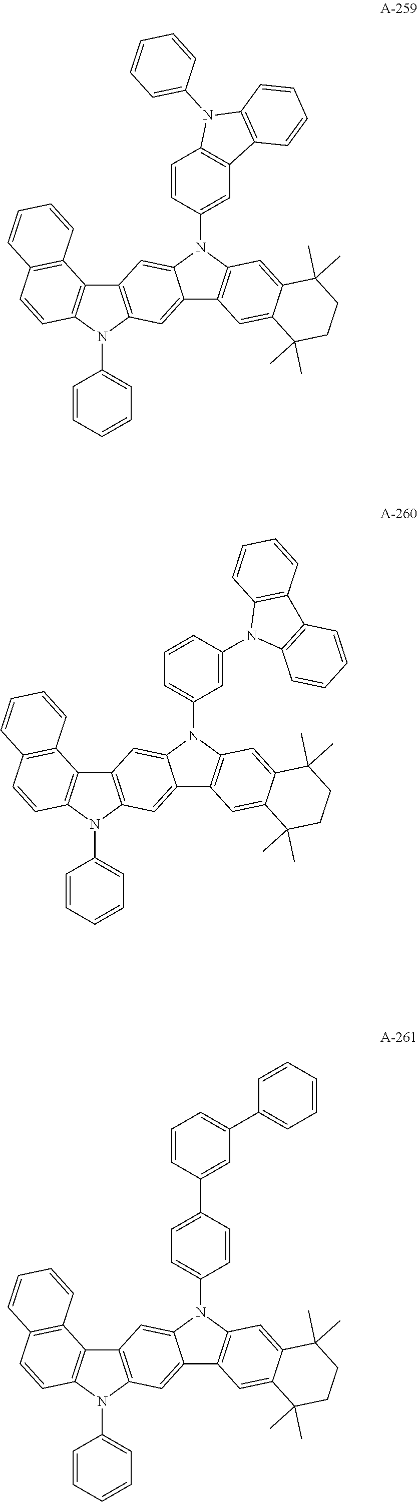 [SMILES: CC1(C)CCC(C)(C)c2cc3c(cc21)c1cc2c(cc1n3-c1ccc3c(c1)c1ccccc1n3-c1ccccc1)c1c3ccccc3ccc1n2-c1ccccc1 ; CC1(C)CCC(C)(C)c2cc3c(cc21)c1cc2c(cc1n3-c1cccc(-n3c4ccccc4c4ccccc43)c1)c1c3ccccc3ccc1n2-c1ccccc1 ; CC1(C)CCC(C)(C)c2cc3c(cc21)c1cc2c(cc1n3-c1ccc(-c3cccc(-c4ccccc4)c3)cc1)c1c3ccccc3ccc1n2-c1ccccc1]

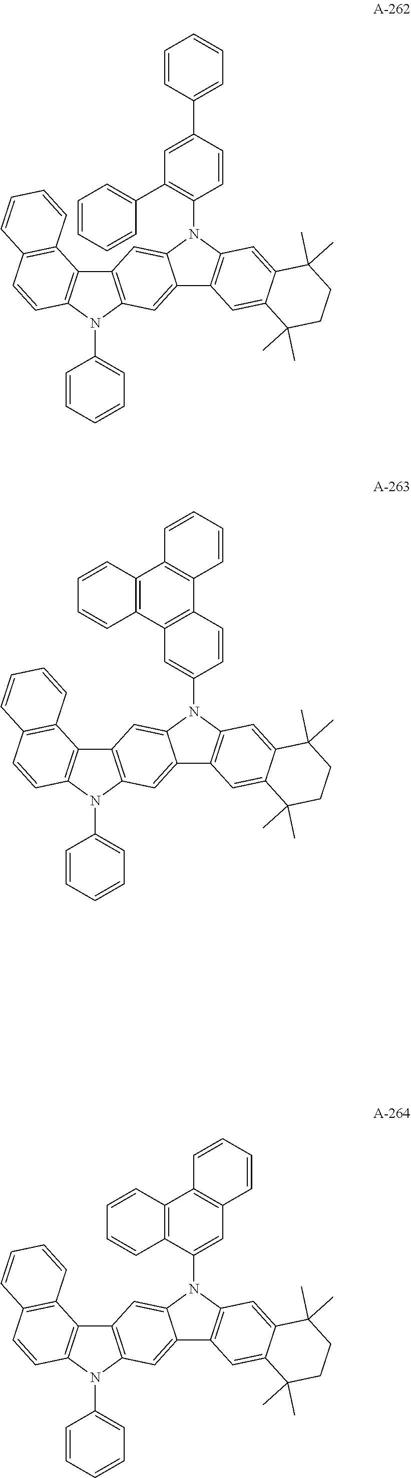 [SMILES: CC1(C)CCC(C)(C)c2cc3c(cc21)c1cc2c(cc1n3-c1ccc(-c3ccccc3)cc1-c1ccccc1)c1c3ccccc3ccc1n2-c1ccccc1 ; CC1(C)CCC(C)(C)c2cc3c(cc21)c1cc2c(cc1n3-c1ccc3c4ccccc4c4ccccc4c3c1)c1c3ccccc3ccc1n2-c1ccccc1 ; CC1(C)CCC(C)(C)c2cc3c(cc21)c1cc2c(cc1n3-c1cc3ccccc3c3ccccc13)c1c3ccccc3ccc1n2-c1ccccc1]

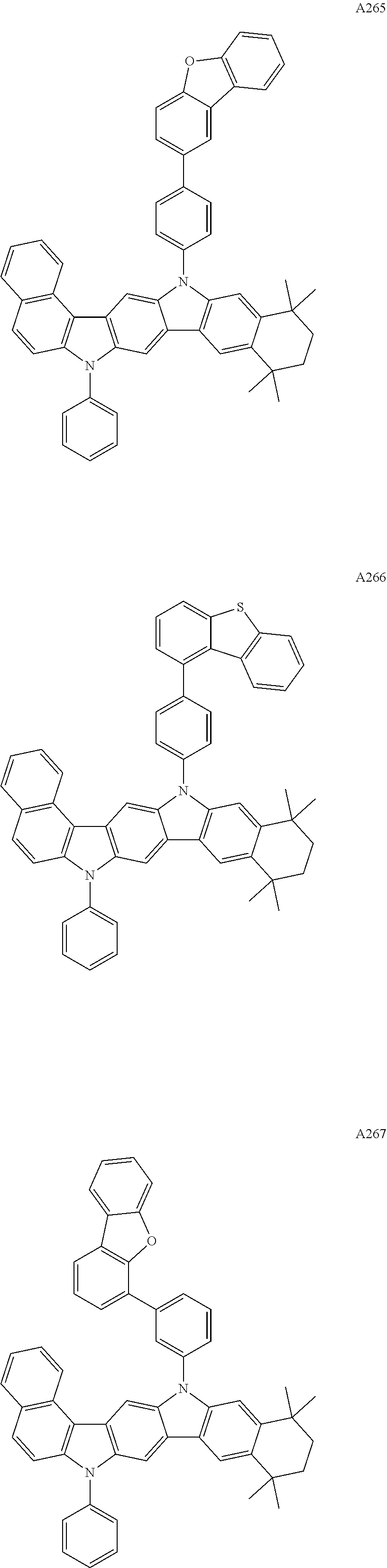 [SMILES: CC1(C)CCC(C)(C)c2cc3c(cc21)c1cc2c(cc1n3-c1ccc(-c3ccc4oc5ccccc5c4c3)cc1)c1c3ccccc3ccc1n2-c1ccccc1 ; CC1(C)CCC(C)(C)c2cc3c(cc21)c1cc2c(cc1n3-c1ccc(-c3cccc4sc5ccccc5c34)cc1)c1c3ccccc3ccc1n2-c1ccccc1 ; CC1(C)CCC(C)(C)c2cc3c(cc21)c1cc2c(cc1n3-c1cccc(-c3cccc4c3oc3ccccc34)c1)c1c3ccccc3ccc1n2-c1ccccc1]

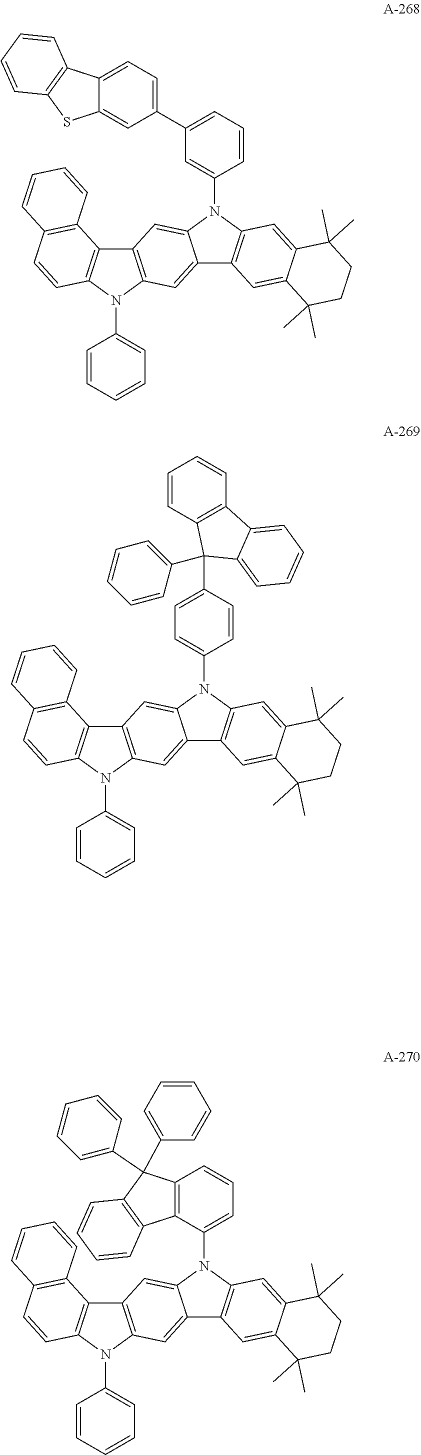 [SMILES: CC1(C)CCC(C)(C)c2cc3c(cc21)c1cc2c(cc1n3-c1cccc(-c3ccc4c(c3)sc3ccccc34)c1)c1c3ccccc3ccc1n2-c1ccccc1 ; CC1(C)CCC(C)(C)c2cc3c(cc21)c1cc2c(cc1n3-c1ccc(C3(c4ccccc4)c4ccccc4-c4ccccc43)cc1)c1c3ccccc3ccc1n2-c1ccccc1 ; CC1(C)CCC(C)(C)c2cc3c(cc21)c1cc2c(cc1n3-c1cccc3c1-c1ccccc1C3(c1ccccc1)c1ccccc1)c1c3ccccc3ccc1n2-c1ccccc1]

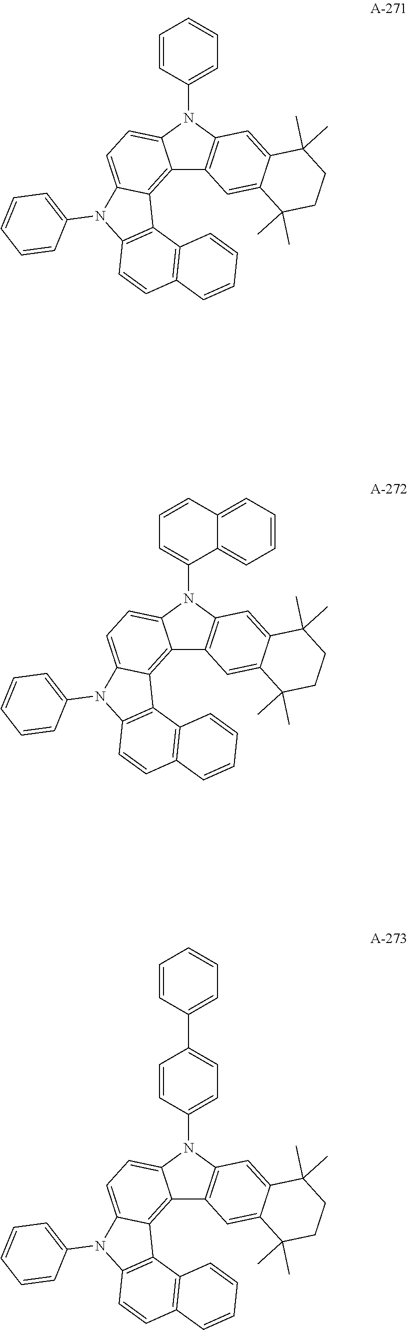 [SMILES: CC1(C)CCC(C)(C)c2cc3c(cc21)c1c2c4c5ccccc5ccc4n(-c4ccccc4)c2ccc1n3-c1ccccc1 ; CC1(C)CCC(C)(C)c2cc3c(cc21)c1c2c4c5ccccc5ccc4n(-c4ccccc4)c2ccc1n3-c1cccc2ccccc12 ; CC1(C)CCC(C)(C)c2cc3c(cc21)c1c2c4c5ccccc5ccc4n(-c4ccccc4)c2ccc1n3-c1ccc(-c2ccccc2)cc1]

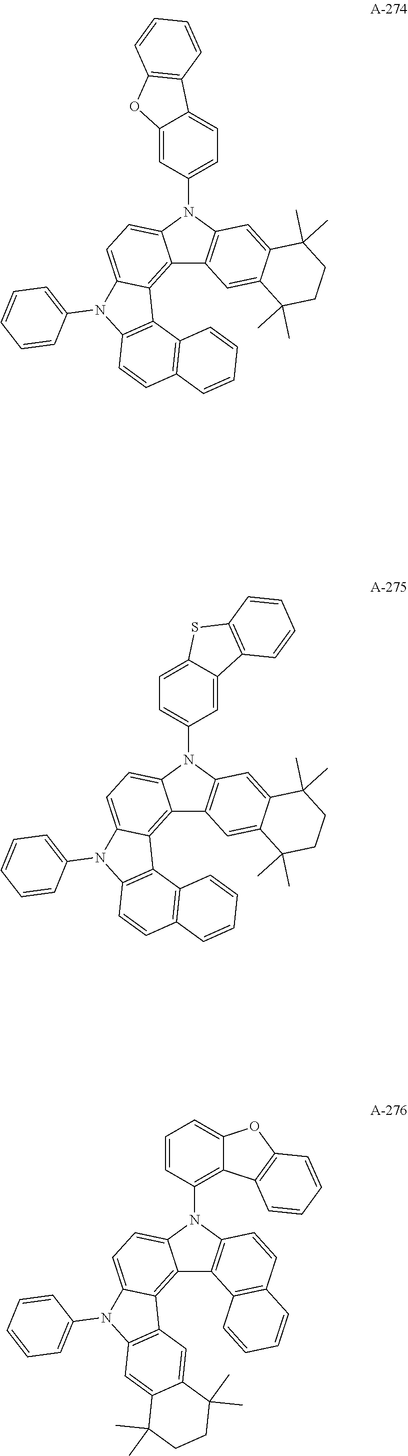 [SMILES: CC1(C)CCC(C)(C)c2cc3c(cc21)c1c2c4c5ccccc5ccc4n(-c4ccccc4)c2ccc1n3-c1ccc2c(c1)oc1ccccc12 ; CC1(C)CCC(C)(C)c2cc3c(cc21)c1c2c4c5ccccc5ccc4n(-c4ccccc4)c2ccc1n3-c1ccc2sc3ccccc3c2c1 ; CC1(C)CCC(C)(C)c2cc3c(cc21)c1c2c4c5ccccc5ccc4n(-c4cccc5oc6ccccc6c45)c2ccc1n3-c1ccccc1]

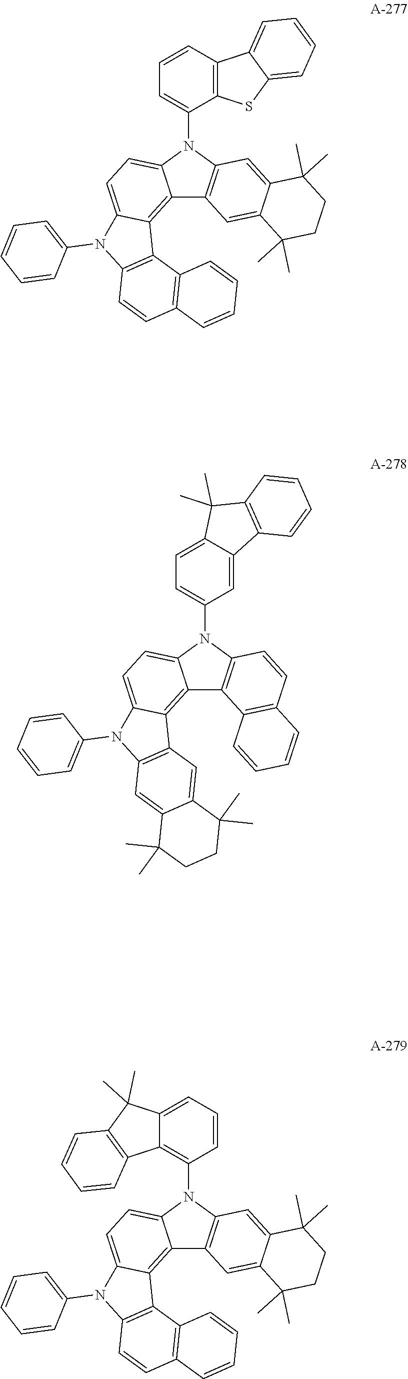 [SMILES: CC1(C)CCC(C)(C)c2cc3c(cc21)c1c2c4c5ccccc5ccc4n(-c4ccccc4)c2ccc1n3-c1cccc2c1sc1ccccc12 ; CC1(C)CCC(C)(C)c2cc3c(cc21)c1c2c4c5ccccc5ccc4n(-c4ccc5c(c4)-c4ccccc4C5(C)C)c2ccc1n3-c1ccccc1 ; CC1(C)CCC(C)(C)c2cc3c(cc21)c1c2c4c5ccccc5ccc4n(-c4ccccc4)c2ccc1n3-c1cccc2c1-c1ccccc1C2(C)C]

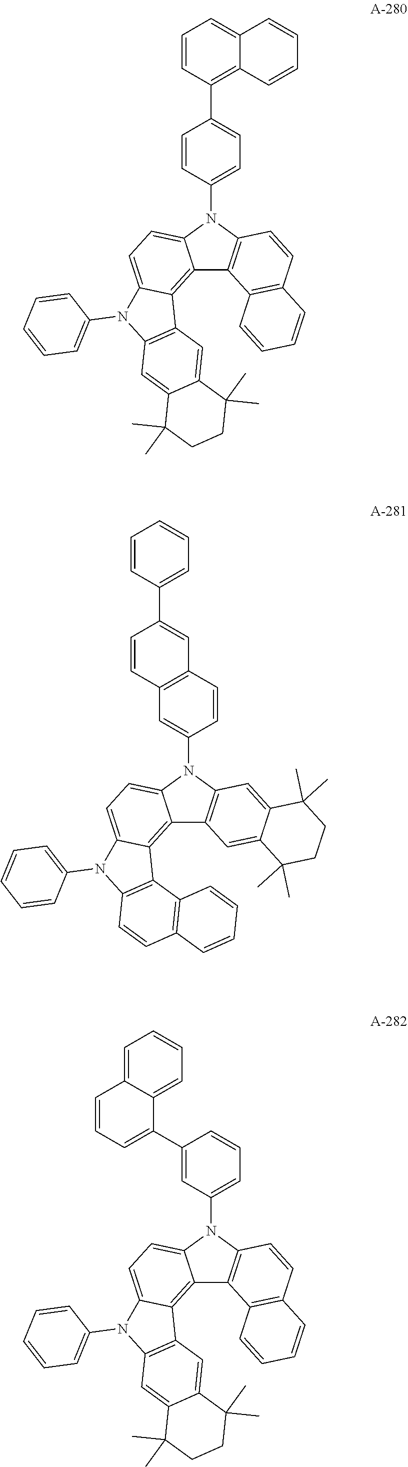 [SMILES: CC1(C)CCC(C)(C)c2cc3c(cc21)c1c2c4c5ccccc5ccc4n(-c4ccc(-c5cccc6ccccc56)cc4)c2ccc1n3-c1ccccc1 ; CC1(C)CCC(C)(C)c2cc3c(cc21)c1c2c4c5ccccc5ccc4n(-c4ccccc4)c2ccc1n3-c1ccc2cc(-c3ccccc3)ccc2c1 ; CC1(C)CCC(C)(C)c2cc3c(cc21)c1c2c4c5ccccc5ccc4n(-c4cccc(-c5cccc6ccccc56)c4)c2ccc1n3-c1ccccc1]

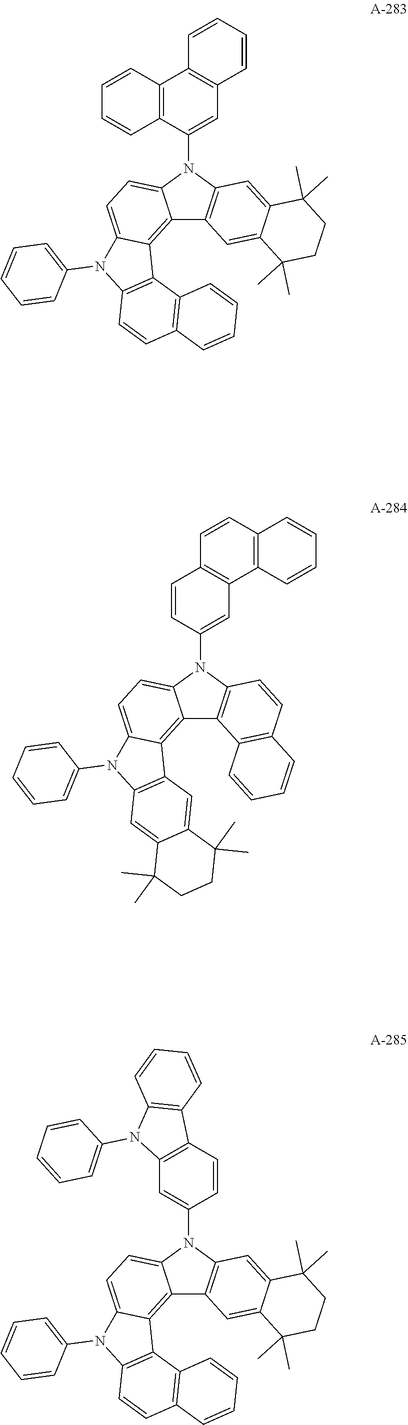 [SMILES: CC1(C)CCC(C)(C)c2cc3c(cc21)c1c2c4c5ccccc5ccc4n(-c4ccccc4)c2ccc1n3-c1cc2ccccc2c2ccccc12 ; CC1(C)CCC(C)(C)c2cc3c(cc21)c1c2c4c5ccccc5ccc4n(-c4ccc5ccc6ccccc6c5c4)c2ccc1n3-c1ccccc1 ; CC1(C)CCC(C)(C)c2cc3c(cc21)c1c2c4c5ccccc5ccc4n(-c4ccccc4)c2ccc1n3-c1ccc2c3ccccc3n(-c3ccccc3)c2c1]

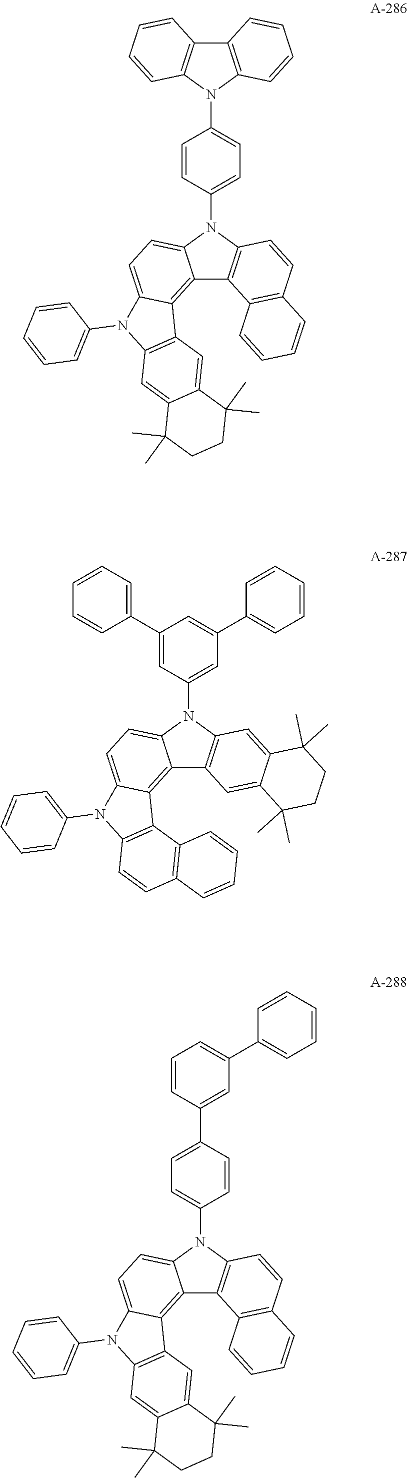 [SMILES: CC1(C)CCC(C)(C)c2cc3c(cc21)c1c2c4c5ccccc5ccc4n(-c4ccc(-n5c6ccccc6c6ccccc65)cc4)c2ccc1n3-c1ccccc1 ; CC1(C)CCC(C)(C)c2cc3c(cc21)c1c2c4c5ccccc5ccc4n(-c4ccccc4)c2ccc1n3-c1cc(-c2ccccc2)cc(-c2ccccc2)c1 ; CC1(C)CCC(C)(C)c2cc3c(cc21)c1c2c4c5ccccc5ccc4n(-c4ccc(-c5cccc(-c6ccccc6)c5)cc4)c2ccc1n3-c1ccccc1]

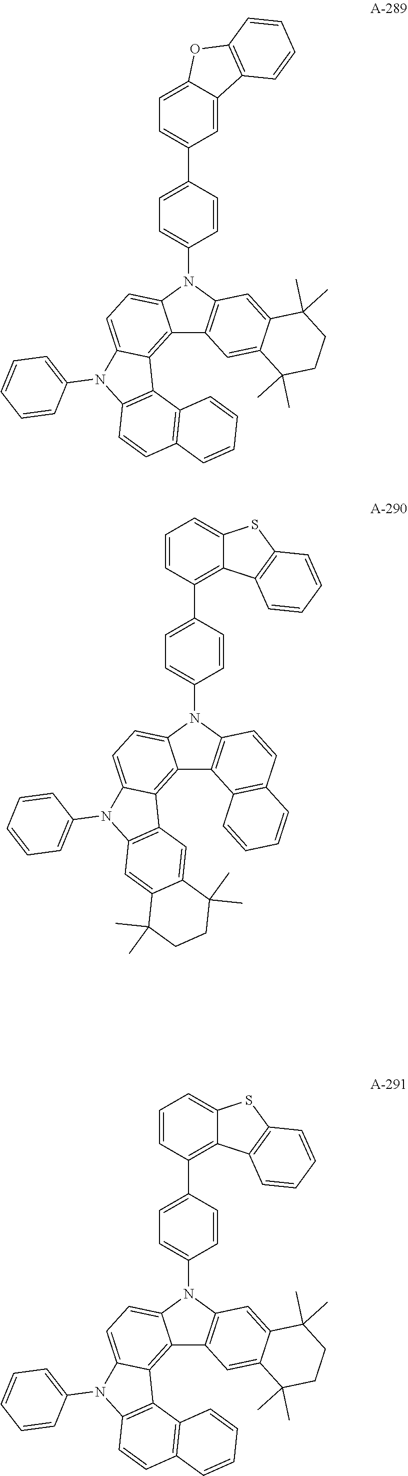 [SMILES: CC1(C)CCC(C)(C)c2cc3c(cc21)c1c2c4c5ccccc5ccc4n(-c4ccccc4)c2ccc1n3-c1ccc(-c2ccc3oc4ccccc4c3c2)cc1 ; CC1(C)CCC(C)(C)c2cc3c(cc21)c1c2c4c5ccccc5ccc4n(-c4ccc(-c5cccc6sc7ccccc7c56)cc4)c2ccc1n3-c1ccccc1 ; CC1(C)CCC(C)(C)c2cc3c(cc21)c1c2c4c5ccccc5ccc4n(-c4ccccc4)c2ccc1n3-c1ccc(-c2cccc3sc4ccccc4c23)cc1]

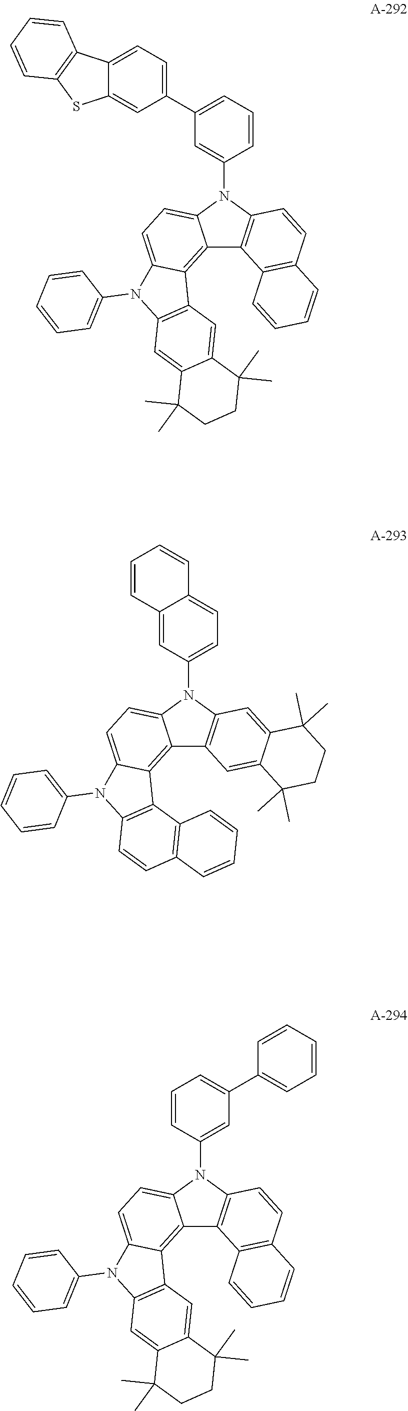 [SMILES: CC1(C)CCC(C)(C)c2cc3c(cc21)c1c2c4c5ccccc5ccc4n(-c4cccc(-c5ccc6c(c5)sc5ccccc56)c4)c2ccc1n3-c1ccccc1 ; CC1(C)CCC(C)(C)c2cc3c(cc21)c1c2c4c5ccccc5ccc4n(-c4ccccc4)c2ccc1n3-c1ccc2ccccc2c1 ; CC1(C)CCC(C)(C)c2cc3c(cc21)c1c2c4c5ccccc5ccc4n(-c4cccc(-c5ccccc5)c4)c2ccc1n3-c1ccccc1]

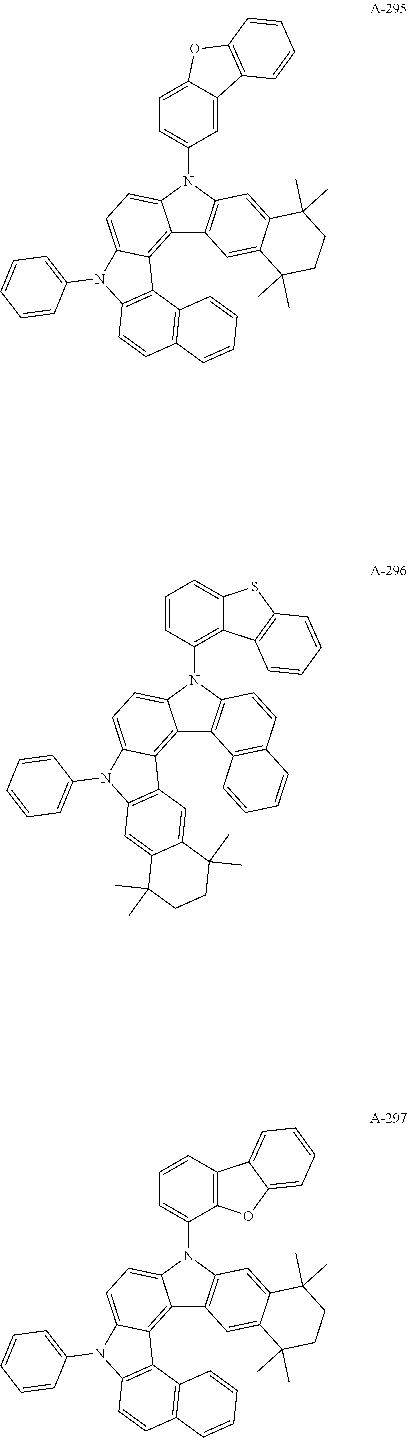 [SMILES: CC1(C)CCC(C)(C)c2cc3c(cc21)c1c2c4c5ccccc5ccc4n(-c4ccccc4)c2ccc1n3-c1ccc2oc3ccccc3c2c1 ; CC1(C)CCC(C)(C)c2cc3c(cc21)c1c2c4c5ccccc5ccc4n(-c4cccc5sc6ccccc6c45)c2ccc1n3-c1ccccc1 ; CC1(C)CCC(C)(C)c2cc3c(cc21)c1c2c4c5ccccc5ccc4n(-c4ccccc4)c2ccc1n3-c1cccc2c1oc1ccccc12]

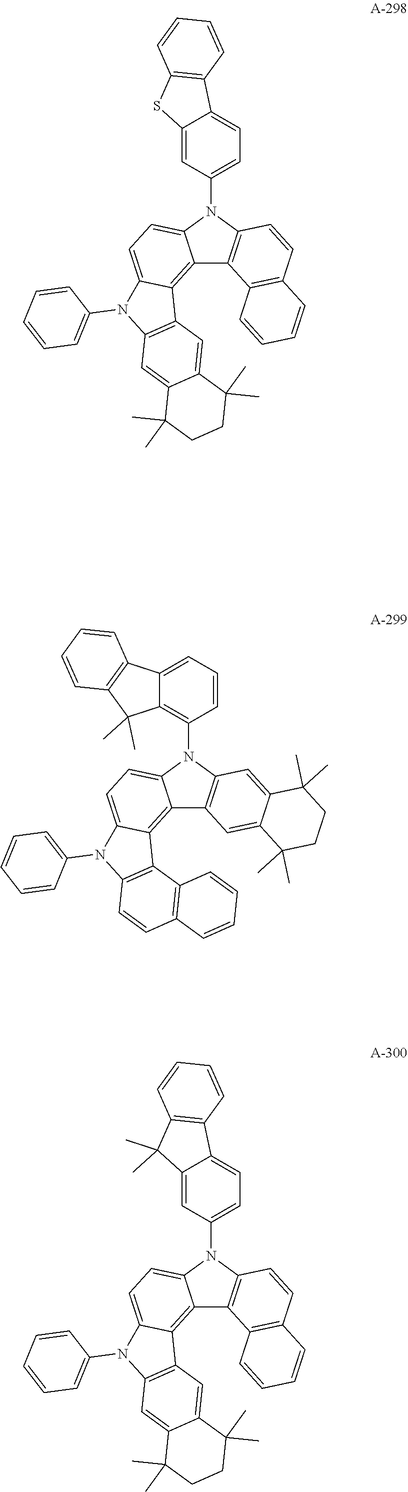 [SMILES: CC1(C)CCC(C)(C)c2cc3c(cc21)c1c2c4c5ccccc5ccc4n(-c4ccc5c(c4)sc4ccccc45)c2ccc1n3-c1ccccc1 ; CC1(C)CCC(C)(C)c2cc3c(cc21)c1c2c4c5ccccc5ccc4n(-c4ccccc4)c2ccc1n3-c1cccc2c1C(C)(C)c1ccccc1-2 ; CC1(C)CCC(C)(C)c2cc3c(cc21)c1c2c4c5ccccc5ccc4n(-c4ccc5c(c4)C(C)(C)c4ccccc4-5)c2ccc1n3-c1ccccc1]

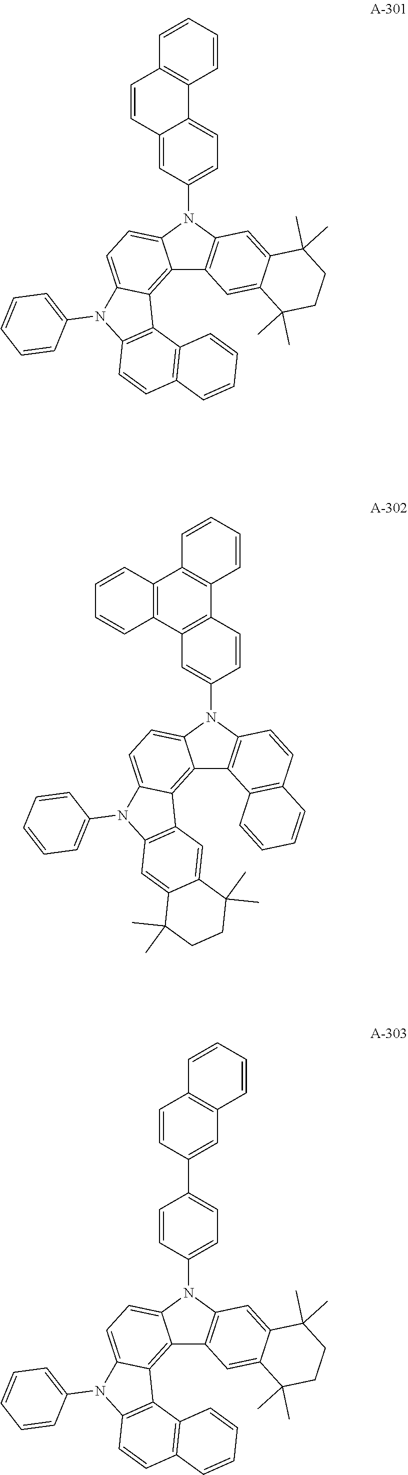 [SMILES: CC1(C)CCC(C)(C)c2cc3c(cc21)c1c2c4c5ccccc5ccc4n(-c4ccccc4)c2ccc1n3-c1ccc2c(ccc3ccccc32)c1 ; CC1(C)CCC(C)(C)c2cc3c(cc21)c1c2c4c5ccccc5ccc4n(-c4ccc5c6ccccc6c6ccccc6c5c4)c2ccc1n3-c1ccccc1 ; CC1(C)CCC(C)(C)c2cc3c(cc21)c1c2c4c5ccccc5ccc4n(-c4ccccc4)c2ccc1n3-c1ccc(-c2ccc3ccccc3c2)cc1]

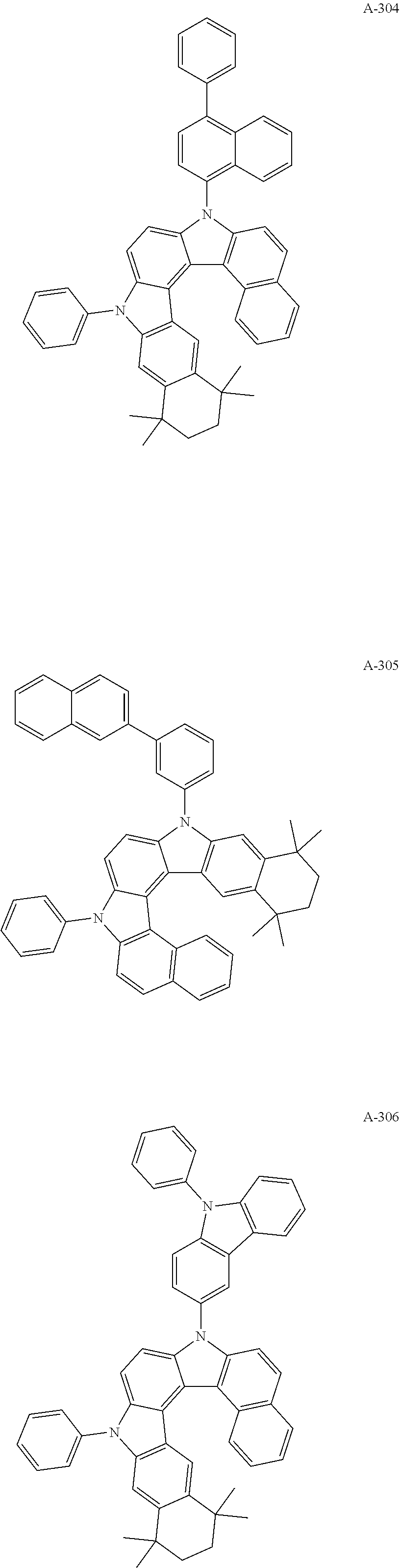 [SMILES: CC1(C)CCC(C)(C)c2cc3c(cc21)c1c2c4c5ccccc5ccc4n(-c4ccc(-c5ccccc5)c5ccccc45)c2ccc1n3-c1ccccc1 ; CC1(C)CCC(C)(C)c2cc3c(cc21)c1c2c4c5ccccc5ccc4n(-c4ccccc4)c2ccc1n3-c1cccc(-c2ccc3ccccc3c2)c1 ; CC1(C)CCC(C)(C)c2cc3c(cc21)c1c2c4c5ccccc5ccc4n(-c4ccc5c(c4)c4ccccc4n5-c4ccccc4)c2ccc1n3-c1ccccc1]

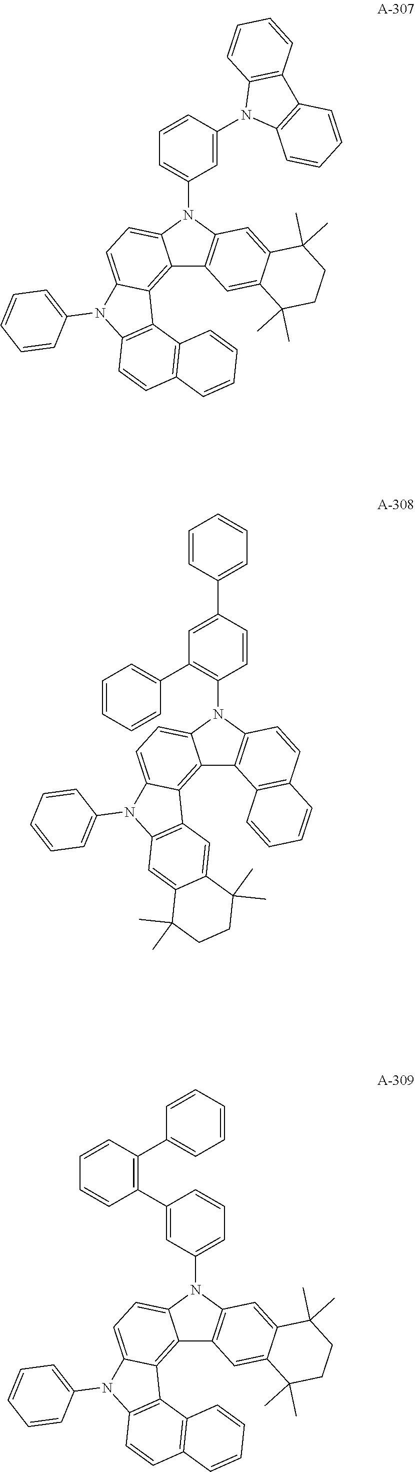 [SMILES: CC1(C)CCC(C)(C)c2cc3c(cc21)c1c2c4c5ccccc5ccc4n(-c4ccccc4)c2ccc1n3-c1cccc(-n2c3ccccc3c3ccccc32)c1 ; CC1(C)CCC(C)(C)c2cc3c(cc21)c1c2c4c5ccccc5ccc4n(-c4ccc(-c5ccccc5)cc4-c4ccccc4)c2ccc1n3-c1ccccc1 ; CC1(C)CCC(C)(C)c2cc3c(cc21)c1c2c4c5ccccc5ccc4n(-c4ccccc4)c2ccc1n3-c1cccc(-c2ccccc2-c2ccccc2)c1]

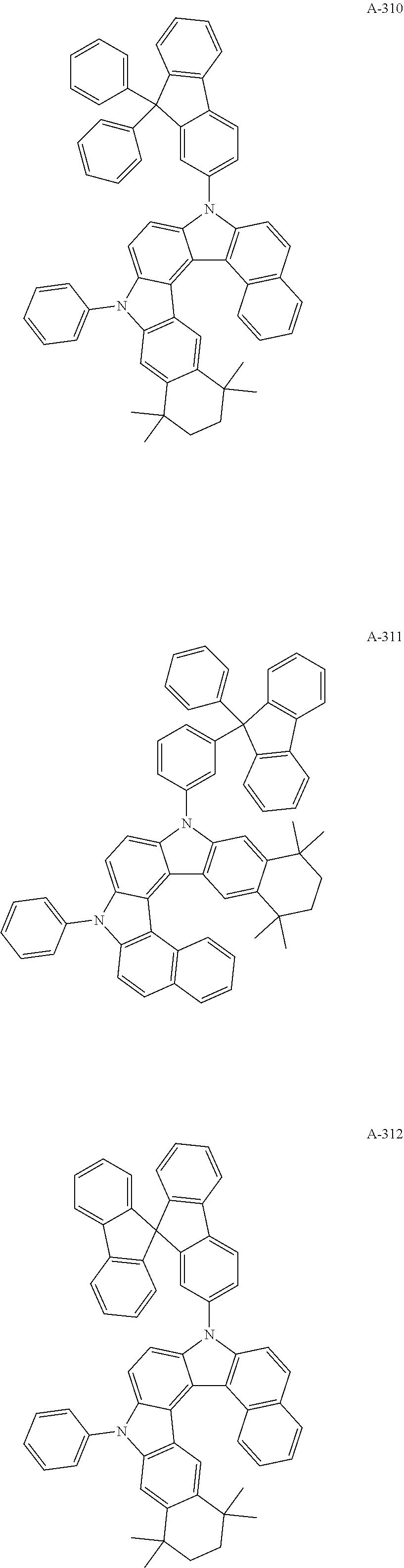 [SMILES: CC1(C)CCC(C)(C)c2cc3c(cc21)c1c2c4c5ccccc5ccc4n(-c4ccc5c(c4)C(c4ccccc4)(c4ccccc4)c4ccccc4-5)c2ccc1n3-c1ccccc1 ; CC1(C)CCC(C)(C)c2cc3c(cc21)c1c2c4c5ccccc5ccc4n(-c4ccccc4)c2ccc1n3-c1cccc(C2(c3ccccc3)c3ccccc3-c3ccccc32)c1 ; CC1(C)CCC(C)(C)c2cc3c(cc21)c1c2c4c5ccccc5ccc4n(-c4ccc5c(c4)C4(c6ccccc6-c6ccccc64)c4ccccc4-5)c2ccc1n3-c1ccccc1]

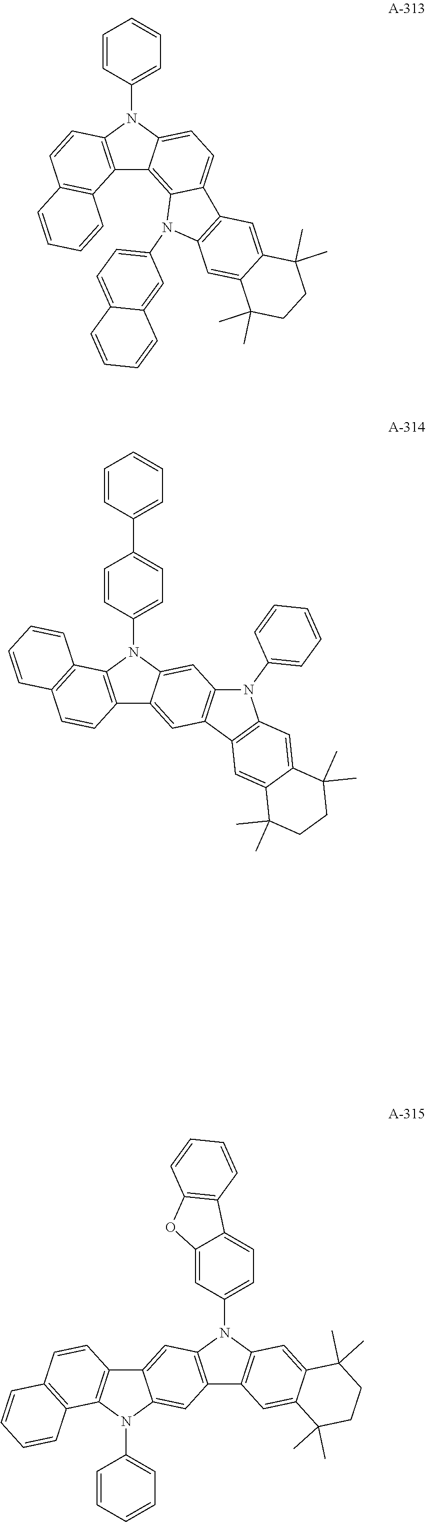 [SMILES: CC1(C)CCC(C)(C)c2cc3c(cc21)c1ccc2c(c4c5ccccc5ccc4n2-c2ccccc2)c1n3-c1ccc2ccccc2c1 ; CC1(C)CCC(C)(C)c2cc3c(cc21)c1cc2c4ccc5ccccc5c4n(-c4ccc(-c5ccccc5)cc4)c2cc1n3-c1ccccc1 ; CC1(C)CCC(C)(C)c2cc3c(cc21)c1cc2c(cc1n3-c1ccc3c(c1)oc1ccccc13)c1ccc3ccccc3c1n2-c1ccccc1]

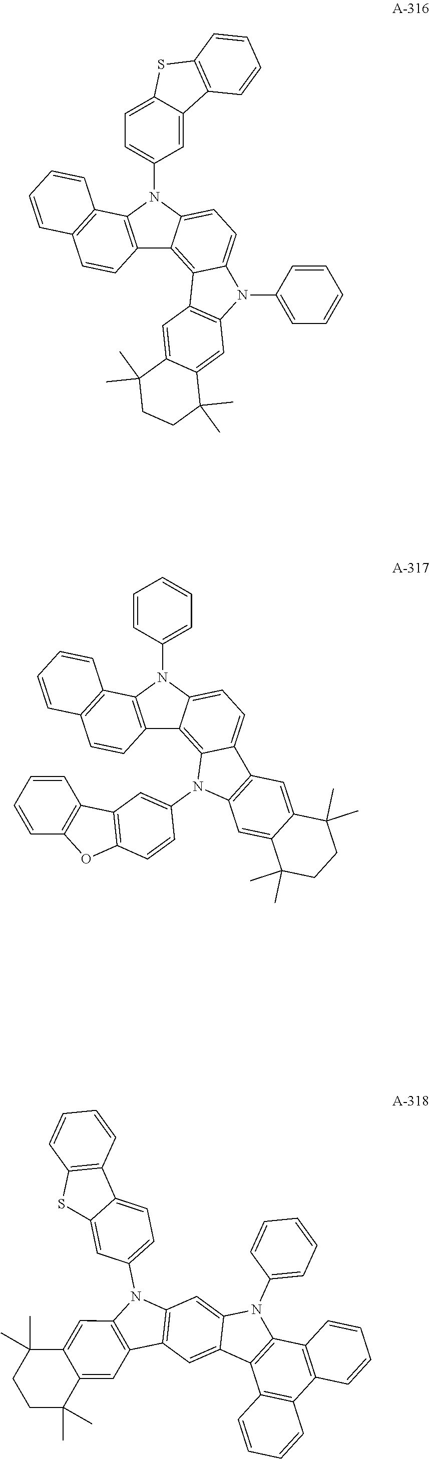

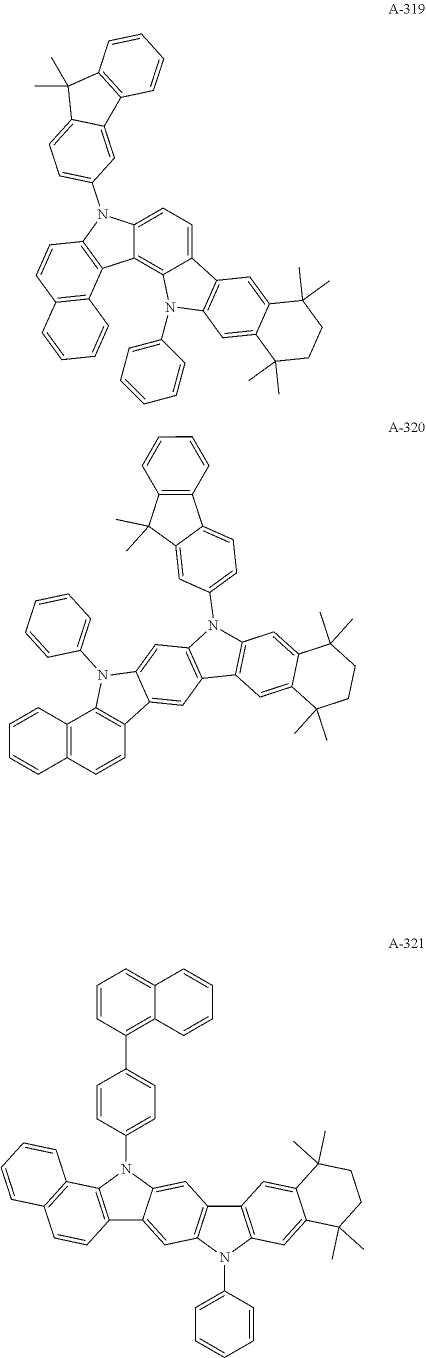

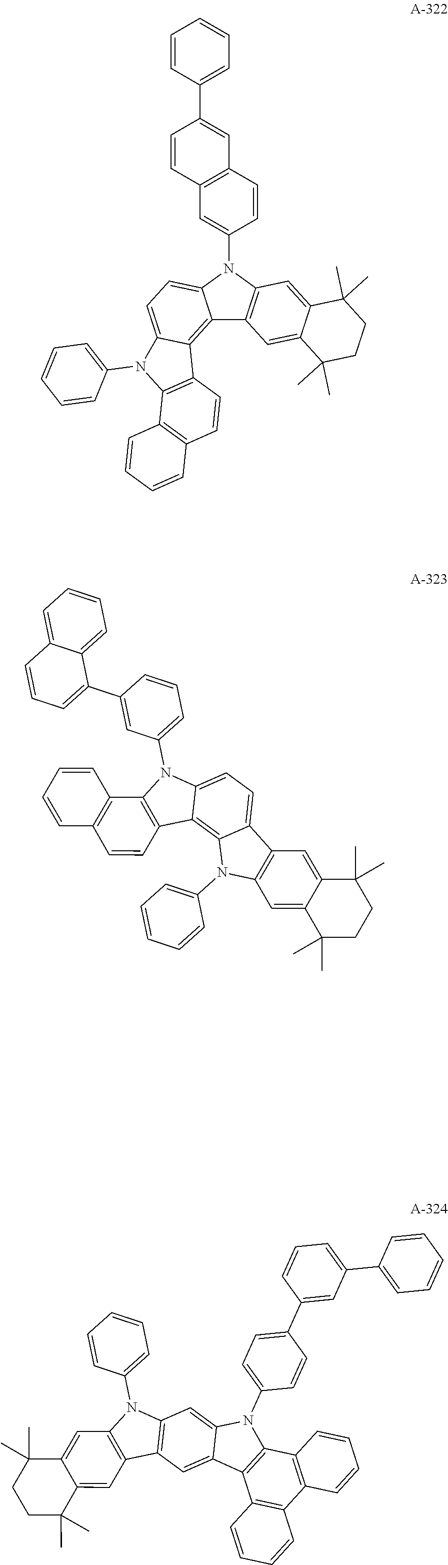

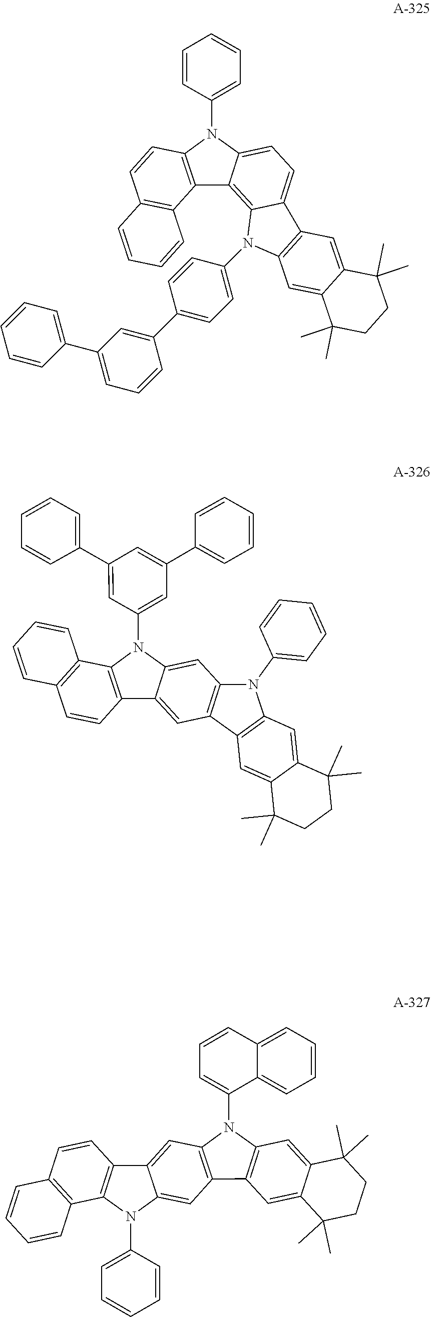 [SMILES: CC1(C)CCC(C)(C)c2cc3c(cc21)c1ccc2c(c4c5ccccc5ccc4n2-c2ccccc2)c1n3-c1ccc(-c2cccc(-c3ccccc3)c2)cc1 ; CC1(C)CCC(C)(C)c2cc3c(cc21)c1cc2c4ccc5ccccc5c4n(-c4cc(-c5ccccc5)cc(-c5ccccc5)c4)c2cc1n3-c1ccccc1 ; CC1(C)CCC(C)(C)c2cc3c(cc21)c1cc2c(cc1n3-c1cccc3ccccc13)c1ccc3ccccc3c1n2-c1ccccc1]

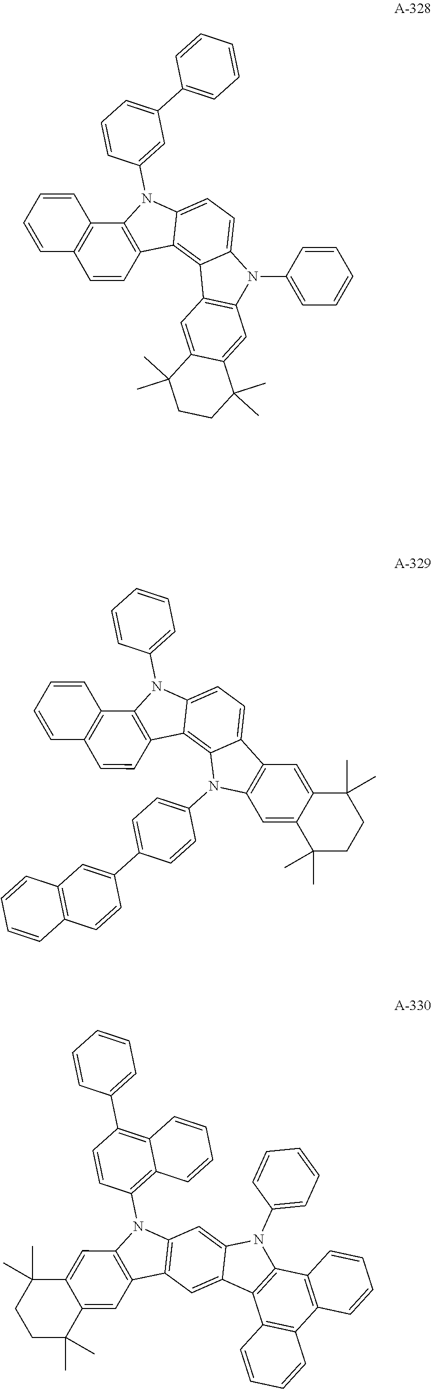

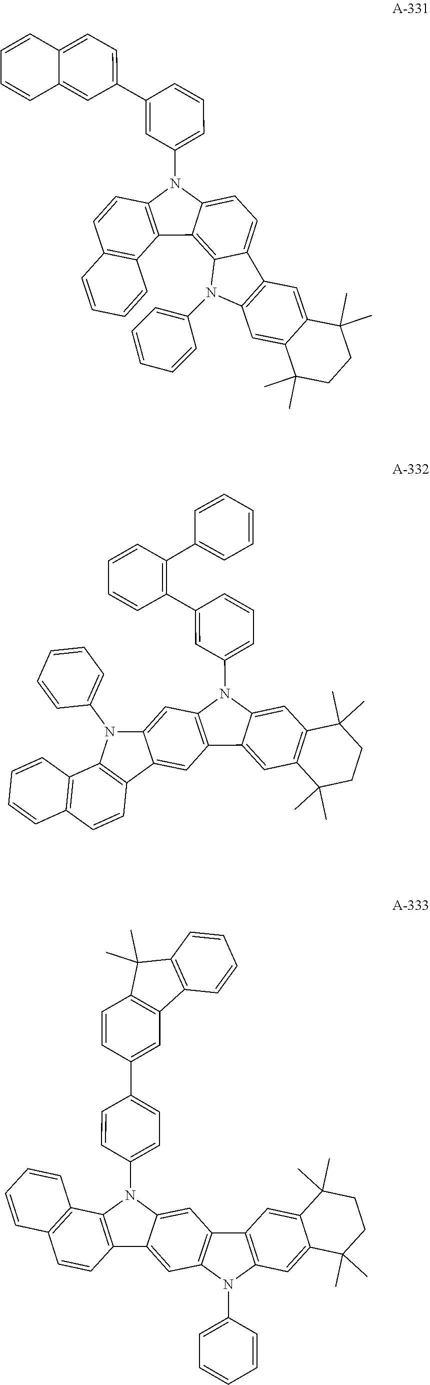 [SMILES: CC1(C)CCC(C)(C)c2cc3c(cc21)c1ccc2c(c4c5ccccc5ccc4n2-c2cccc(-c4ccc5ccccc5c4)c2)c1n3-c1ccccc1 ; CC1(C)CCC(C)(C)c2cc3c(cc21)c1cc2c4ccc5ccccc5c4n(-c4ccccc4)c2cc1n3-c1cccc(-c2ccccc2-c2ccccc2)c1 ; CC1(C)CCC(C)(C)c2cc3c(cc21)c1cc2c(cc1n3-c1ccccc1)c1ccc3ccccc3c1n2-c1ccc(-c2ccc3c(c2)-c2ccccc2C3(C)C)cc1]

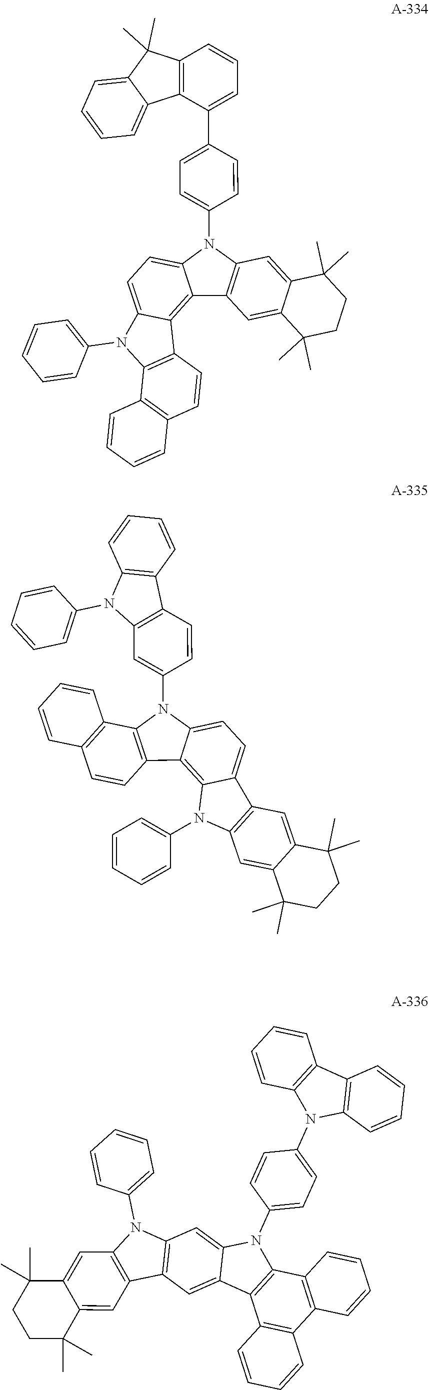

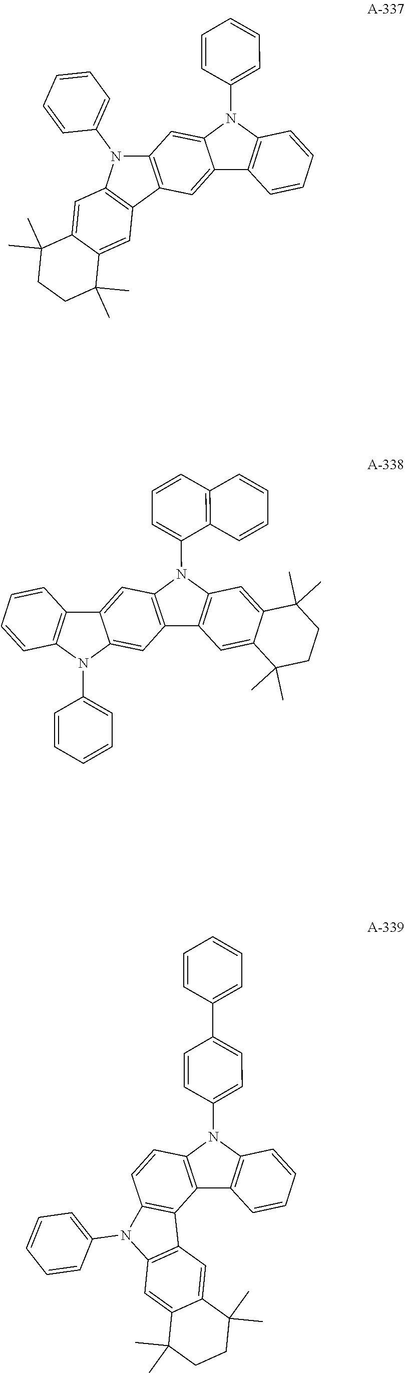 [SMILES: CC1(C)CCC(C)(C)c2cc3c(cc21)c1cc2c4ccccc4n(-c4ccccc4)c2cc1n3-c1ccccc1 ; CC1(C)CCC(C)(C)c2cc3c(cc21)c1cc2c(cc1n3-c1cccc3ccccc13)c1ccccc1n2-c1ccccc1 ; CC1(C)CCC(C)(C)c2cc3c(cc21)c1c2c4ccccc4n(-c4ccc(-c5ccccc5)cc4)c2ccc1n3-c1ccccc1]

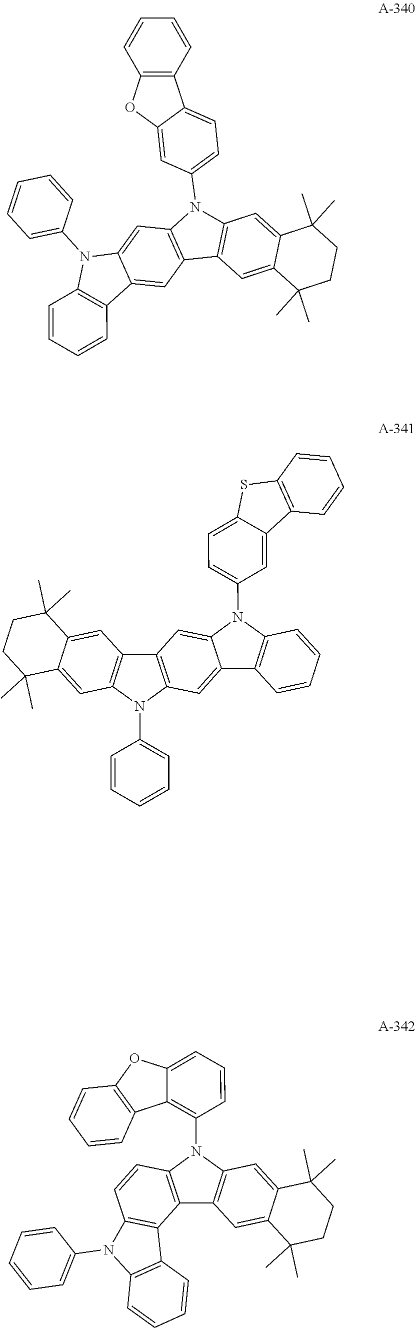 [SMILES: CC1(C)CCC(C)(C)c2cc3c(cc21)c1cc2c4ccccc4n(-c4ccccc4)c2cc1n3-c1ccc2c(c1)oc1ccccc12 ; CC1(C)CCC(C)(C)c2cc3c(cc21)c1cc2c(cc1n3-c1ccccc1)c1ccccc1n2-c1ccc2sc3ccccc3c2c1 ; CC1(C)CCC(C)(C)c2cc3c(cc21)c1c2c4ccccc4n(-c4ccccc4)c2ccc1n3-c1cccc2oc3ccccc3c12]

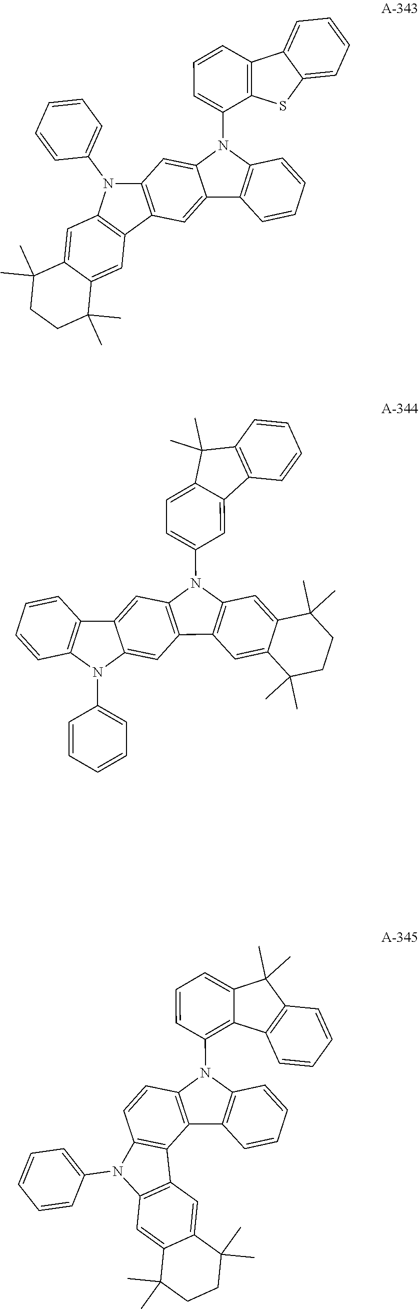 [SMILES: CC1(C)CCC(C)(C)c2cc3c(cc21)c1cc2c4ccccc4n(-c4cccc5c4sc4ccccc45)c2cc1n3-c1ccccc1 ; CC1(C)CCC(C)(C)c2cc3c(cc21)c1cc2c(cc1n3-c1ccc3c(c1)-c1ccccc1C3(C)C)c1ccccc1n2-c1ccccc1 ; CC1(C)CCC(C)(C)c2cc3c(cc21)c1c2c4ccccc4n(-c4cccc5c4-c4ccccc4C5(C)C)c2ccc1n3-c1ccccc1]

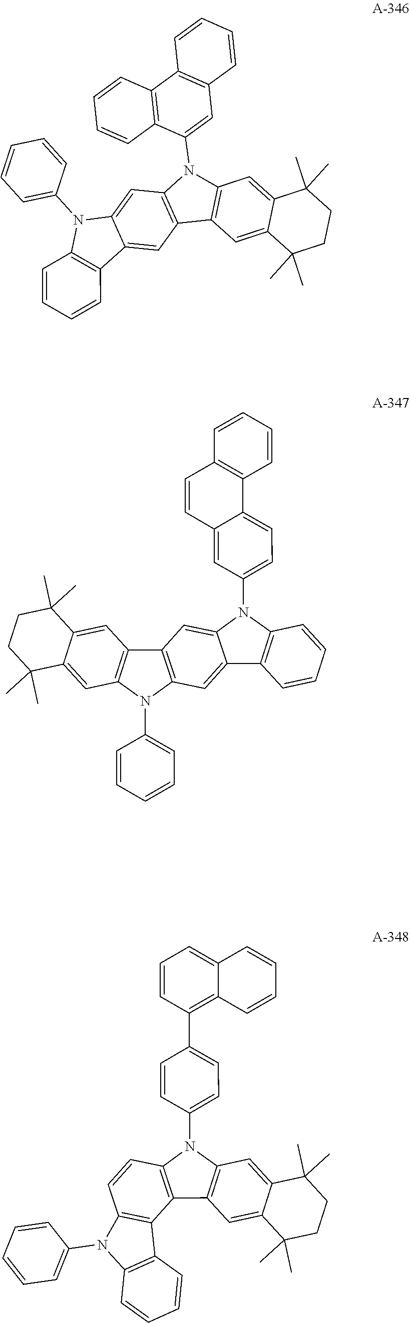 [SMILES: CC1(C)CCC(C)(C)c2cc3c(cc21)c1cc2c4ccccc4n(-c4ccccc4)c2cc1n3-c1cc2ccccc2c2ccccc12 ; CC1(C)CCC(C)(C)c2cc3c(cc21)c1cc2c(cc1n3-c1ccccc1)c1ccccc1n2-c1ccc2c(ccc3ccccc32)c1 ; CC1(C)CCC(C)(C)c2cc3c(cc21)c1c2c4ccccc4n(-c4ccccc4)c2ccc1n3-c1ccc(-c2cccc3ccccc23)cc1]

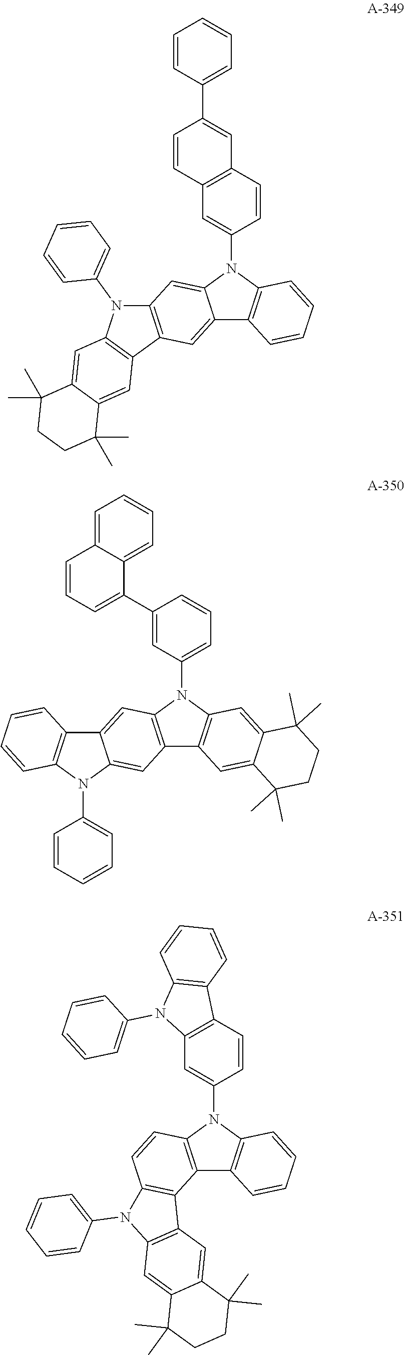

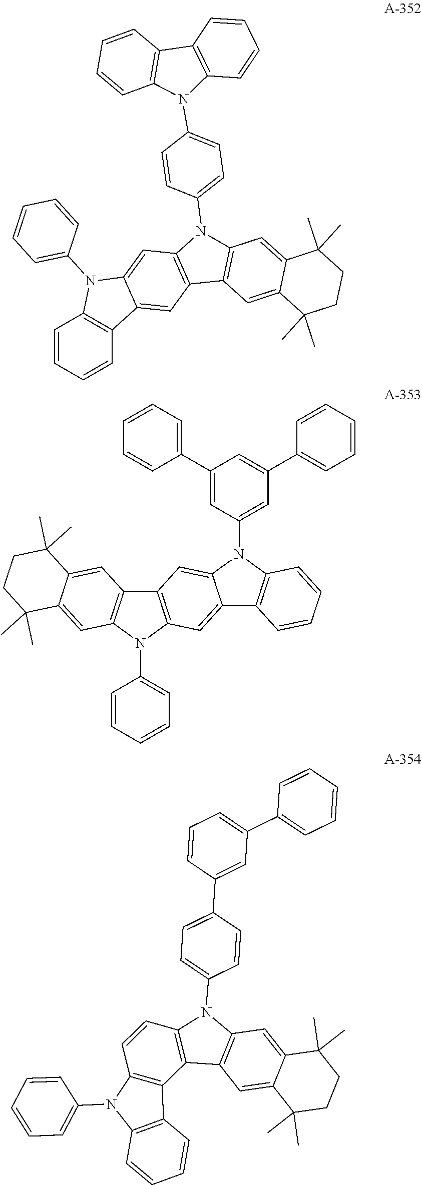 [SMILES: CC1(C)CCC(C)(C)c2cc3c(cc21)c1cc2c4ccccc4n(-c4ccccc4)c2cc1n3-c1ccc(-n2c3ccccc3c3ccccc32)cc1 ; CC1(C)CCC(C)(C)c2cc3c(cc21)c1cc2c(cc1n3-c1ccccc1)c1ccccc1n2-c1cc(-c2ccccc2)cc(-c2ccccc2)c1 ; CC1(C)CCC(C)(C)c2cc3c(cc21)c1c2c4ccccc4n(-c4ccccc4)c2ccc1n3-c1ccc(-c2cccc(-c3ccccc3)c2)cc1]

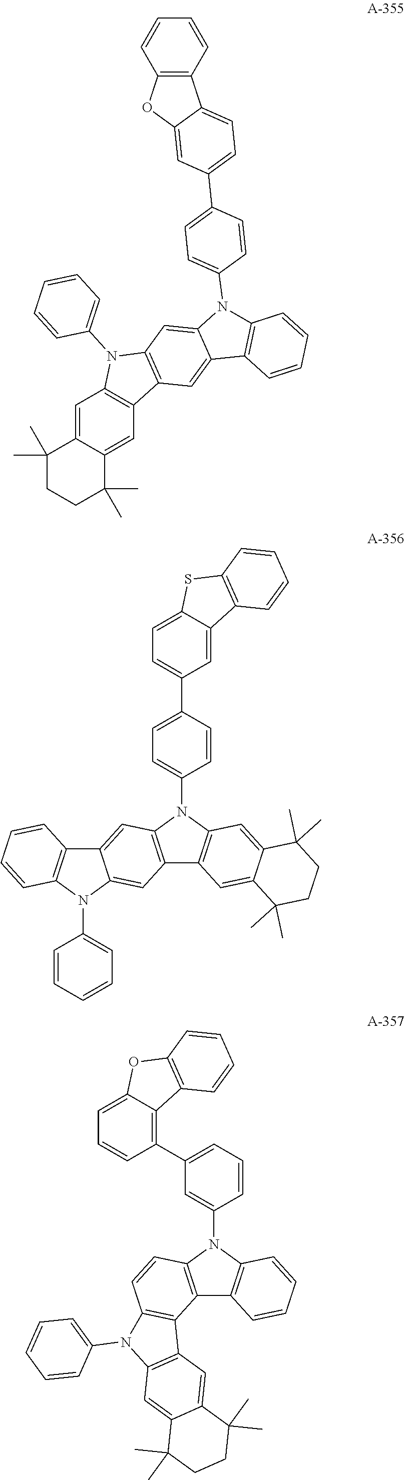 [SMILES: CC1(C)CCC(C)(C)c2cc3c(cc21)c1cc2c4ccccc4n(-c4ccc(-c5ccc6c(c5)oc5ccccc56)cc4)c2cc1n3-c1ccccc1 ; CC1(C)CCC(C)(C)c2cc3c(cc21)c1cc2c(cc1n3-c1ccc(-c3ccc4sc5ccccc5c4c3)cc1)c1ccccc1n2-c1ccccc1 ; CC1(C)CCC(C)(C)c2cc3c(cc21)c1c2c4ccccc4n(-c4cccc(-c5cccc6oc7ccccc7c56)c4)c2ccc1n3-c1ccccc1]

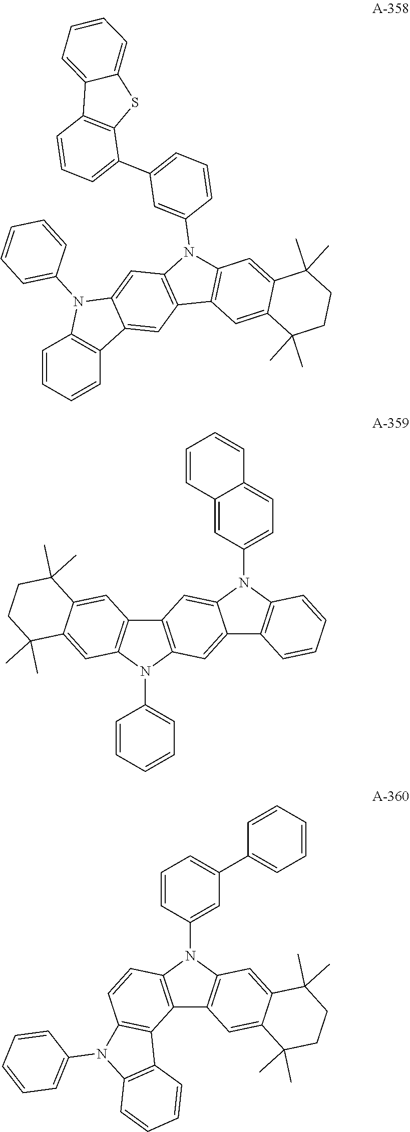 [SMILES: CC1(C)CCC(C)(C)c2cc3c(cc21)c1cc2c4ccccc4n(-c4ccccc4)c2cc1n3-c1cccc(-c2cccc3c2sc2ccccc23)c1 ; CC1(C)CCC(C)(C)c2cc3c(cc21)c1cc2c(cc1n3-c1ccccc1)c1ccccc1n2-c1ccc2ccccc2c1 ; CC1(C)CCC(C)(C)c2cc3c(cc21)c1c2c4ccccc4n(-c4ccccc4)c2ccc1n3-c1cccc(-c2ccccc2)c1]

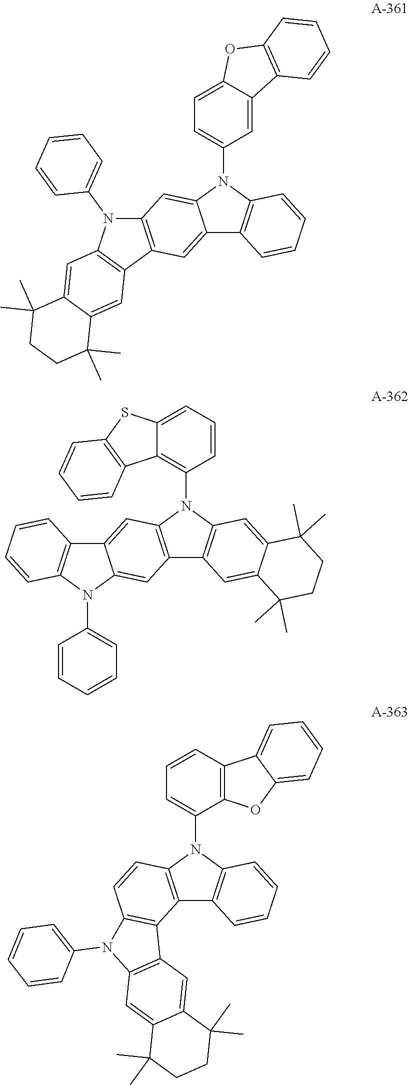

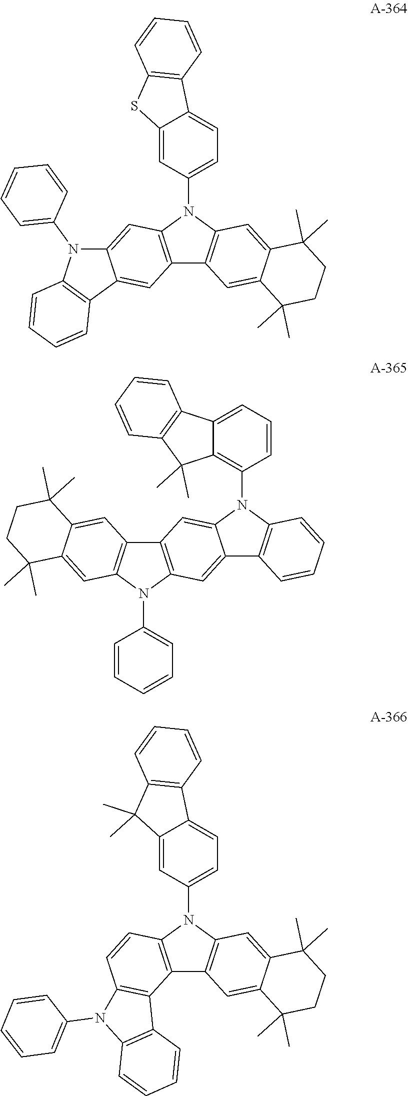

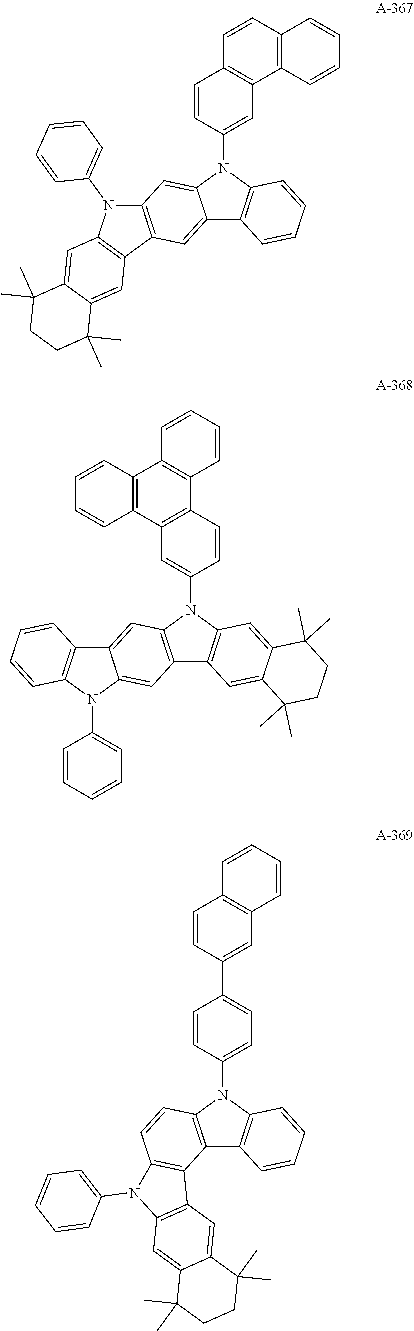 [SMILES: CC1(C)CCC(C)(C)c2cc3c(cc21)c1cc2c4ccccc4n(-c4ccc5ccc6ccccc6c5c4)c2cc1n3-c1ccccc1 ; CC1(C)CCC(C)(C)c2cc3c(cc21)c1cc2c(cc1n3-c1ccc3c4ccccc4c4ccccc4c3c1)c1ccccc1n2-c1ccccc1 ; CC1(C)CCC(C)(C)c2cc3c(cc21)c1c2c4ccccc4n(-c4ccc(-c5ccc6ccccc6c5)cc4)c2ccc1n3-c1ccccc1]

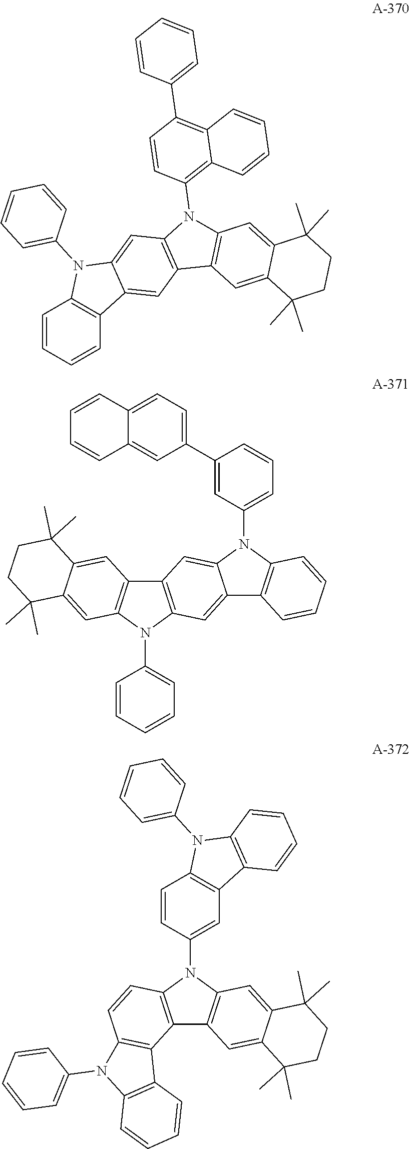

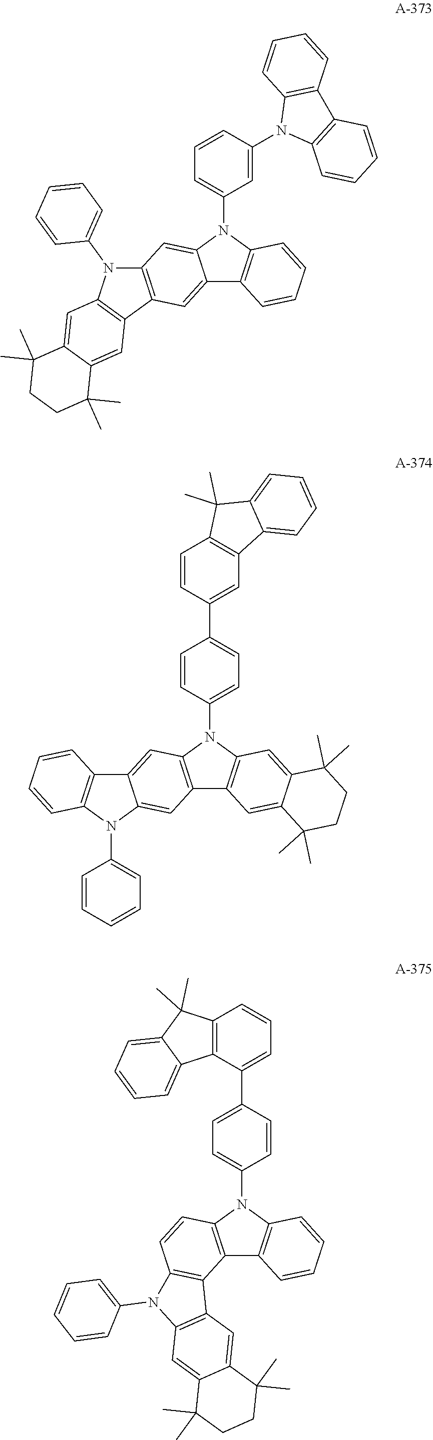 [SMILES: CC1(C)CCC(C)(C)c2cc3c(cc21)c1cc2c4ccccc4n(-c4cccc(-n5c6ccccc6c6ccccc65)c4)c2cc1n3-c1ccccc1 ; CC1(C)CCC(C)(C)c2cc3c(cc21)c1cc2c(cc1n3-c1ccc(-c3ccc4c(c3)-c3ccccc3C4(C)C)cc1)c1ccccc1n2-c1ccccc1 ; CC1(C)CCC(C)(C)c2cc3c(cc21)c1c2c4ccccc4n(-c4ccc(-c5cccc6c5-c5ccccc5C6(C)C)cc4)c2ccc1n3-c1ccccc1]

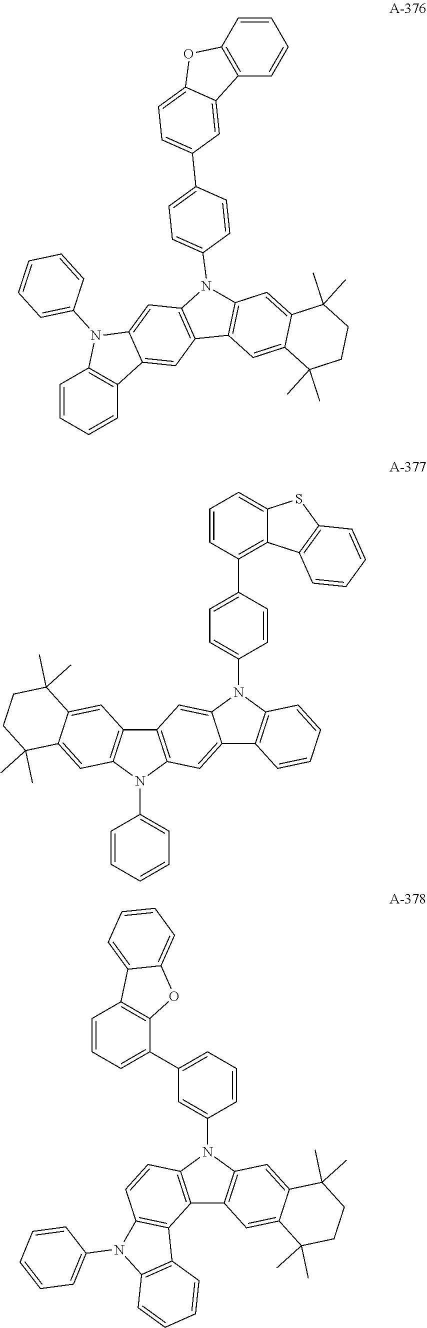

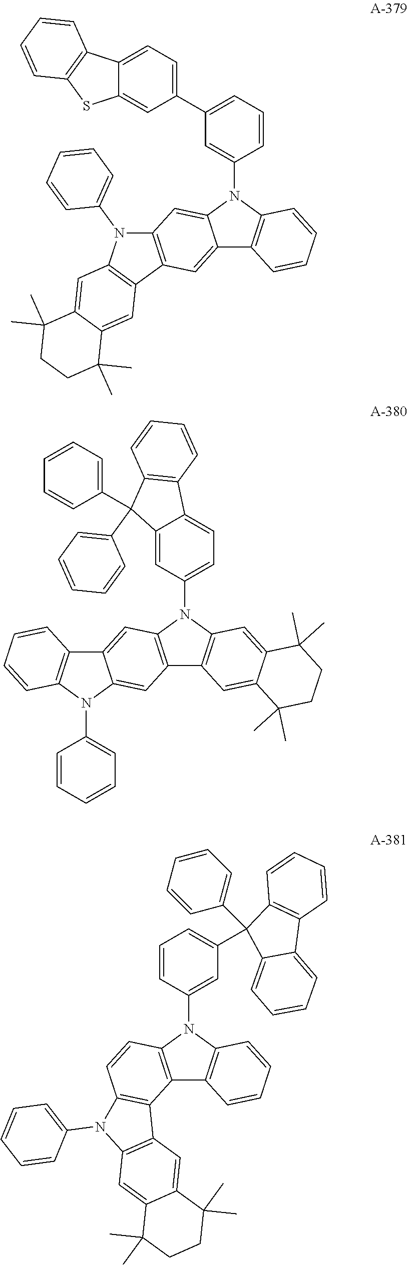 [SMILES: CC1(C)CCC(C)(C)c2cc3c(cc21)c1cc2c4ccccc4n(-c4cccc(-c5ccc6c(c5)sc5ccccc56)c4)c2cc1n3-c1ccccc1 ; CC1(C)CCC(C)(C)c2cc3c(cc21)c1cc2c(cc1n3-c1ccc3c(c1)C(c1ccccc1)(c1ccccc1)c1ccccc1-3)c1ccccc1n2-c1ccccc1 ; CC1(C)CCC(C)(C)c2cc3c(cc21)c1c2c4ccccc4n(-c4cccc(C5(c6ccccc6)c6ccccc6-c6ccccc65)c4)c2ccc1n3-c1ccccc1]

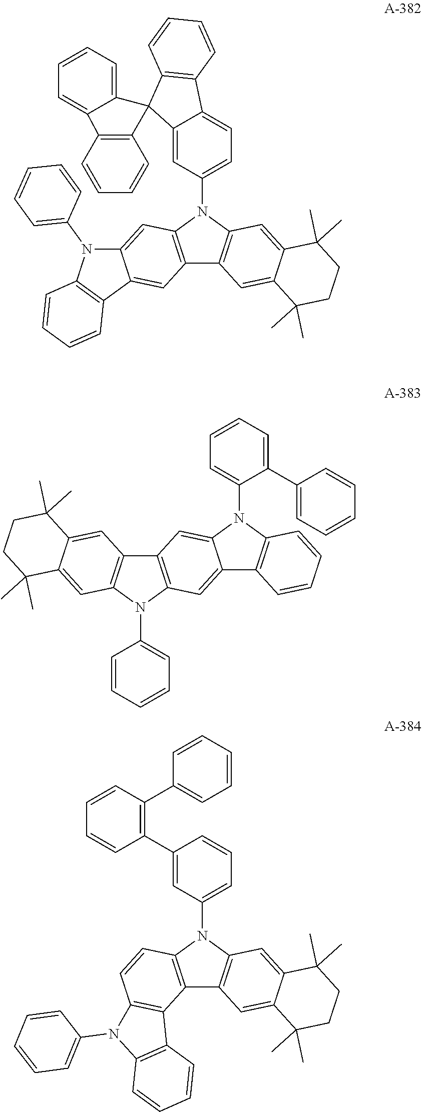

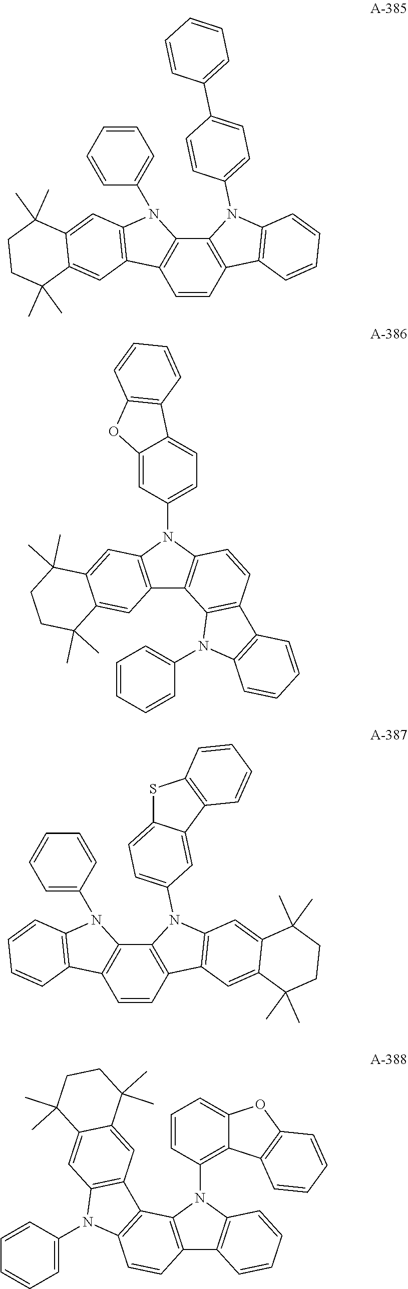 [SMILES: CC1(C)CCC(C)(C)c2cc3c(cc21)c1ccc2c4ccccc4n(-c4ccc(-c5ccccc5)cc4)c2c1n3-c1ccccc1 ; CC1(C)CCC(C)(C)c2cc3c(cc21)c1c(ccc2c4ccccc4n(-c4ccccc4)c21)n3-c1ccc2c(c1)oc1ccccc12 ; CC1(C)CCC(C)(C)c2cc3c(cc21)c1ccc2c4ccccc4n(-c4ccccc4)c2c1n3-c1ccc2sc3ccccc3c2c1 ; CC1(C)CCC(C)(C)c2cc3c(cc21)c1c(ccc2c4ccccc4n(-c4cccc5oc6ccccc6c45)c21)n3-c1ccccc1]

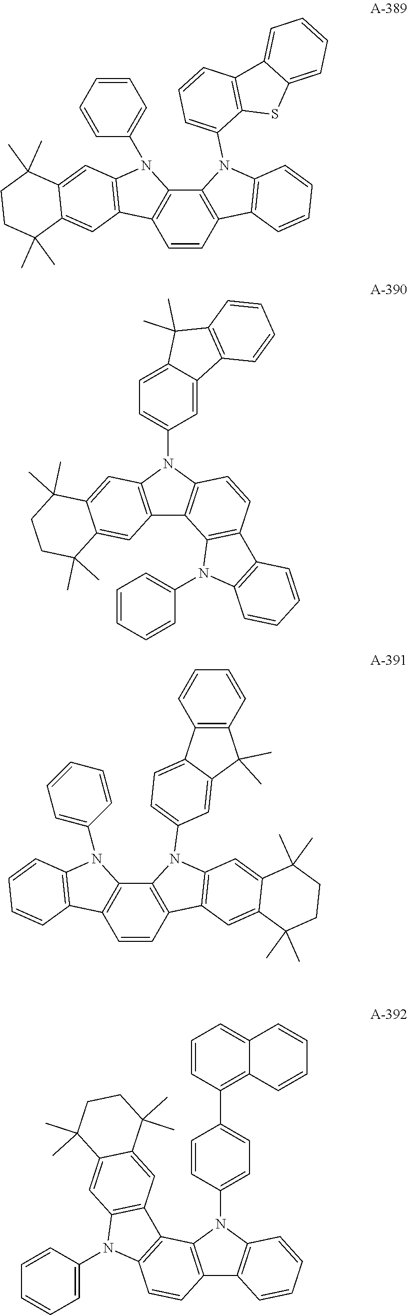 [SMILES: CC1(C)CCC(C)(C)c2cc3c(cc21)c1ccc2c4ccccc4n(-c4cccc5c4sc4ccccc45)c2c1n3-c1ccccc1 ; CC1(C)CCC(C)(C)c2cc3c(cc21)c1c(ccc2c4ccccc4n(-c4ccccc4)c21)n3-c1ccc2c(c1)-c1ccccc1C2(C)C ; CC1(C)CCC(C)(C)c2cc3c(cc21)c1ccc2c4ccccc4n(-c4ccccc4)c2c1n3-c1ccc2c(c1)C(C)(C)c1ccccc1-2 ; CC1(C)CCC(C)(C)c2cc3c(cc21)c1c(ccc2c4ccccc4n(-c4ccc(-c5cccc6ccccc56)cc4)c21)n3-c1ccccc1]

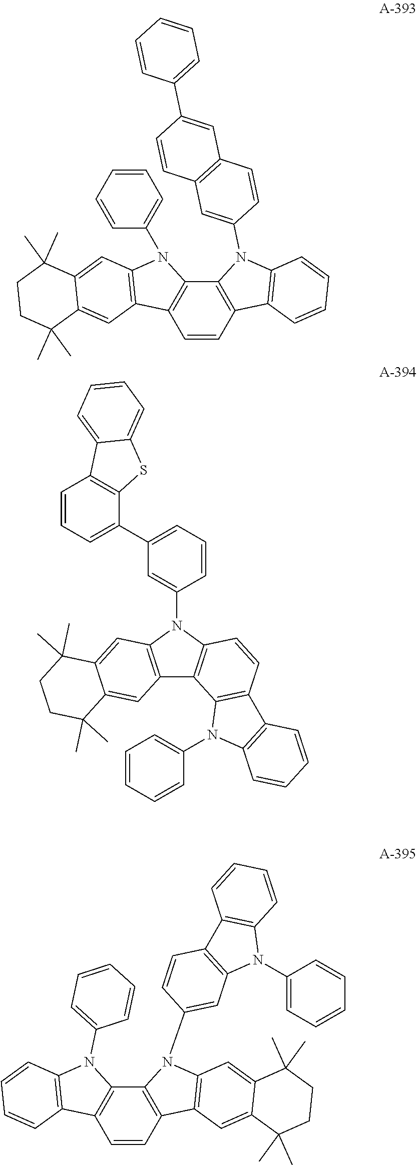

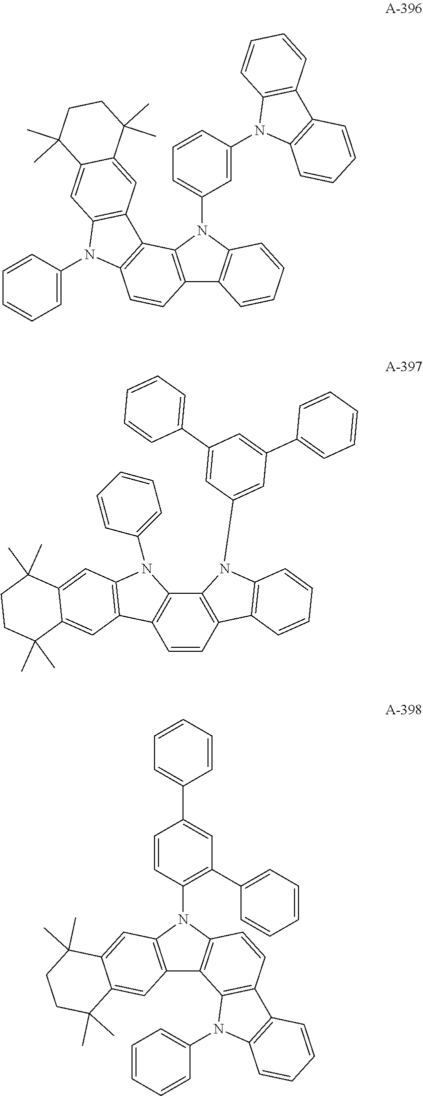 [SMILES: CC1(C)CCC(C)(C)c2cc3c(cc21)c1c(ccc2c4ccccc4n(-c4cccc(-n5c6ccccc6c6ccccc65)c4)c21)n3-c1ccccc1 ; CC1(C)CCC(C)(C)c2cc3c(cc21)c1ccc2c4ccccc4n(-c4cc(-c5ccccc5)cc(-c5ccccc5)c4)c2c1n3-c1ccccc1 ; CC1(C)CCC(C)(C)c2cc3c(cc21)c1c(ccc2c4ccccc4n(-c4ccccc4)c21)n3-c1ccc(-c2ccccc2)cc1-c1ccccc1]

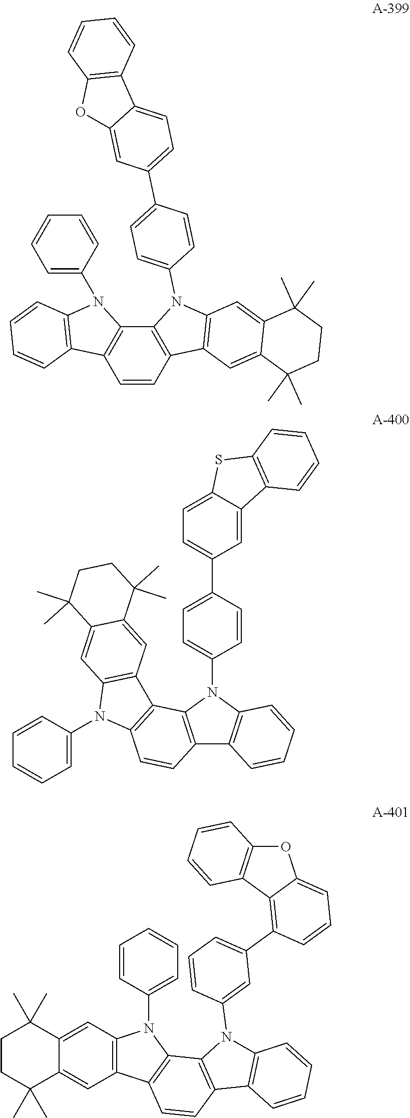

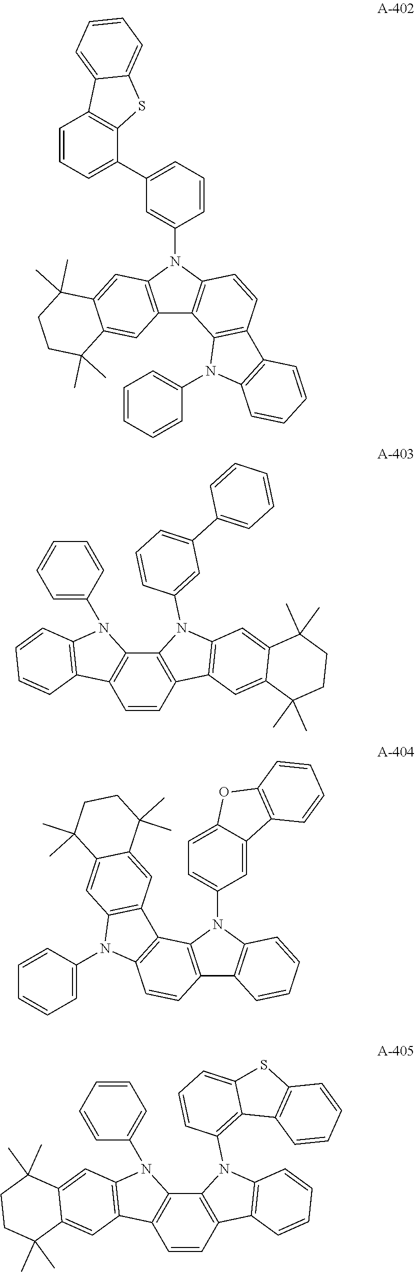

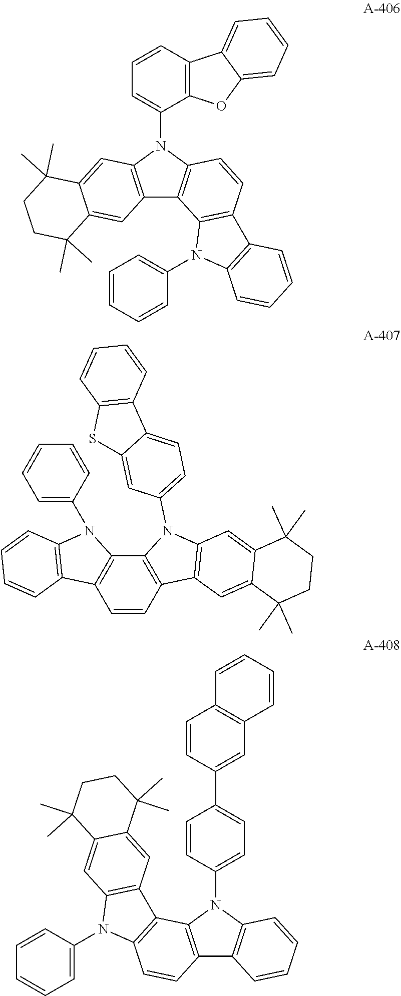

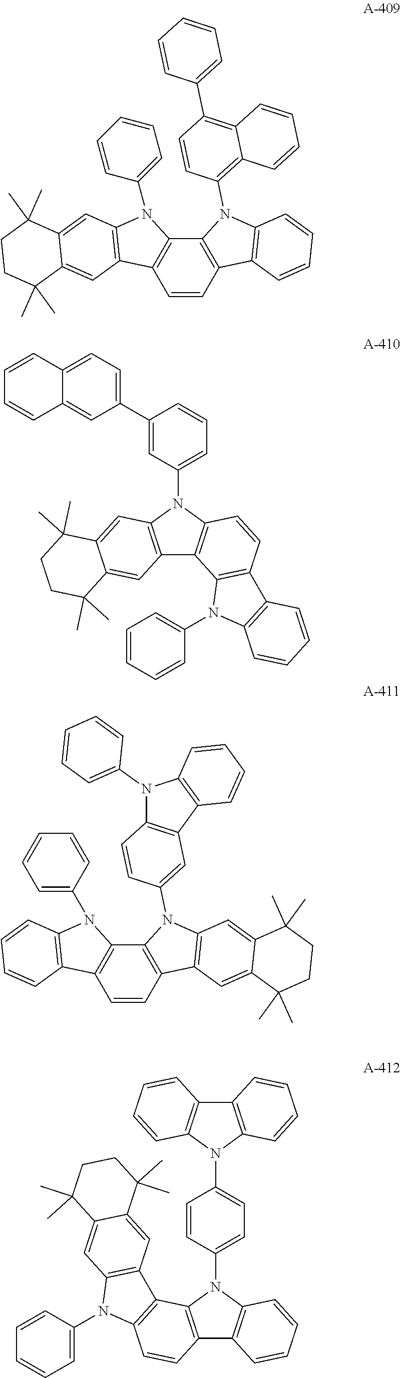

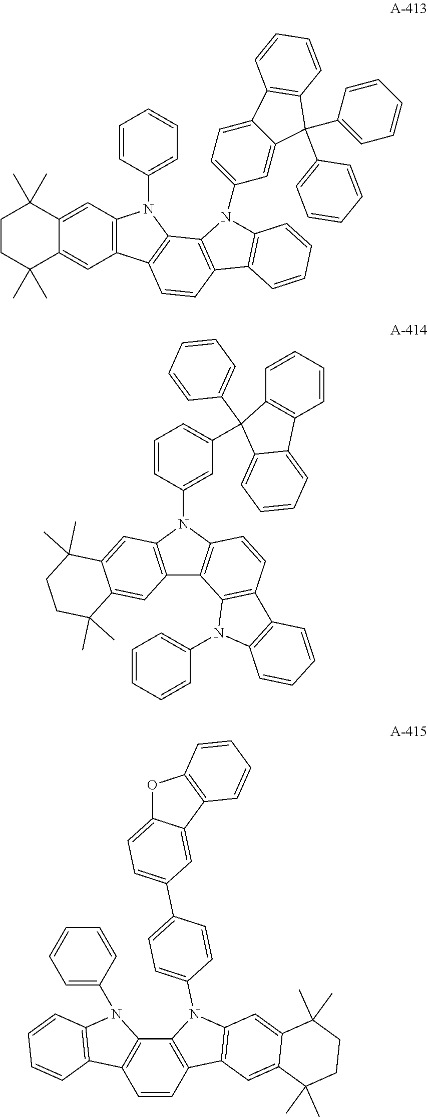 [SMILES: CC1(C)CCC(C)(C)c2cc3c(cc21)c1ccc2c4ccccc4n(-c4ccc5c(c4)C(c4ccccc4)(c4ccccc4)c4ccccc4-5)c2c1n3-c1ccccc1 ; CC1(C)CCC(C)(C)c2cc3c(cc21)c1c(ccc2c4ccccc4n(-c4ccccc4)c21)n3-c1cccc(C2(c3ccccc3)c3ccccc3-c3ccccc32)c1 ; CC1(C)CCC(C)(C)c2cc3c(cc21)c1ccc2c4ccccc4n(-c4ccccc4)c2c1n3-c1ccc(-c2ccc3oc4ccccc4c3c2)cc1]

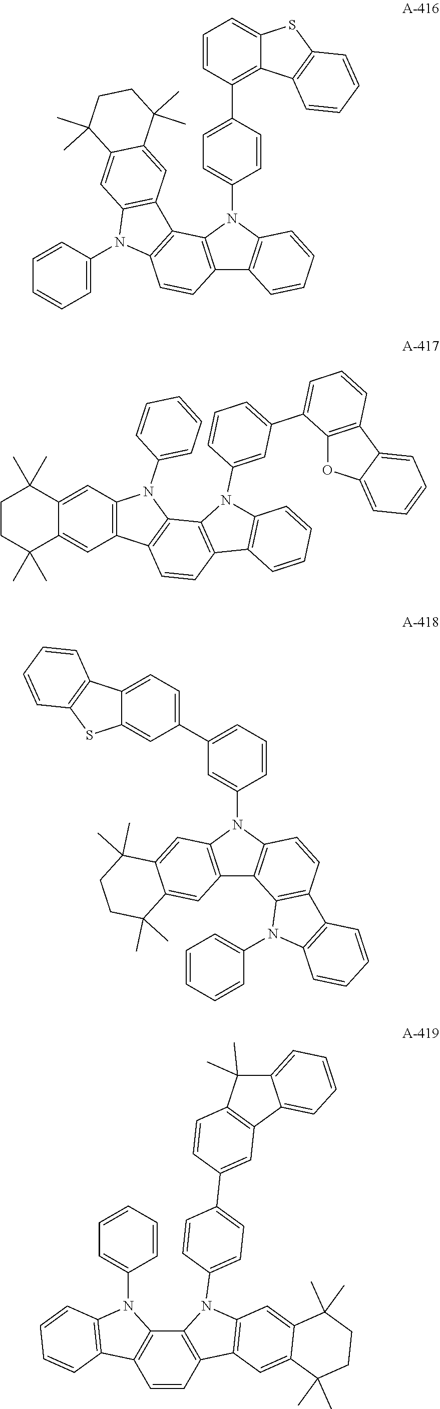

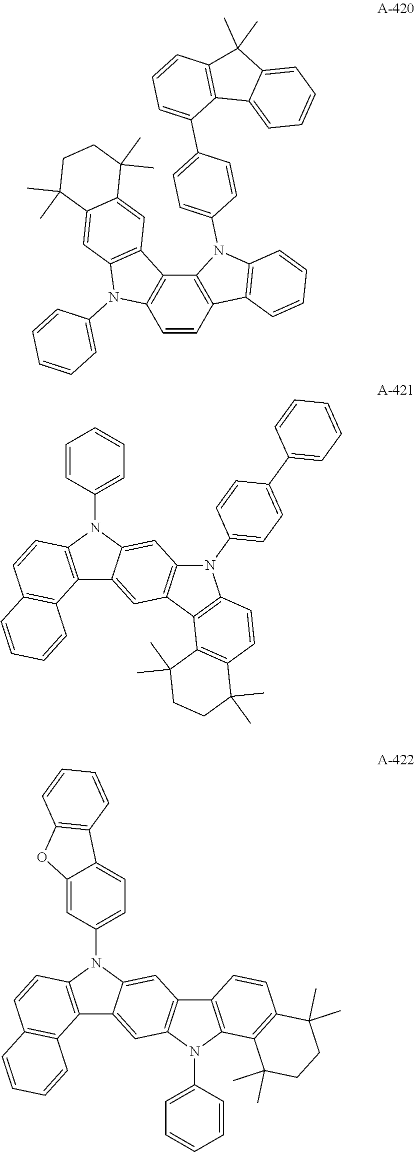 [SMILES: CC1(C)CCC(C)(C)c2cc3c(cc21)c1c(ccc2c4ccccc4n(-c4ccc(-c5cccc6c5-c5ccccc5C6(C)C)cc4)c21)n3-c1ccccc1 ; CC1(C)CCC(C)(C)c2c1ccc1c2c2cc3c4c5ccccc5ccc4n(-c4ccccc4)c3cc2n1-c1ccc(-c2ccccc2)cc1 ; CC1(C)CCC(C)(C)c2c1ccc1c3cc4c(cc3n(-c3ccccc3)c21)c1c2ccccc2ccc1n4-c1ccc2c(c1)oc1ccccc12]

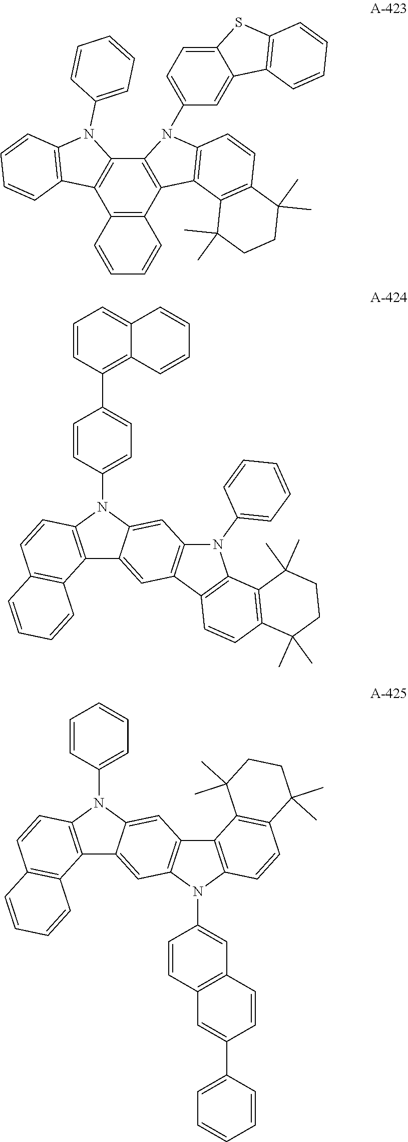

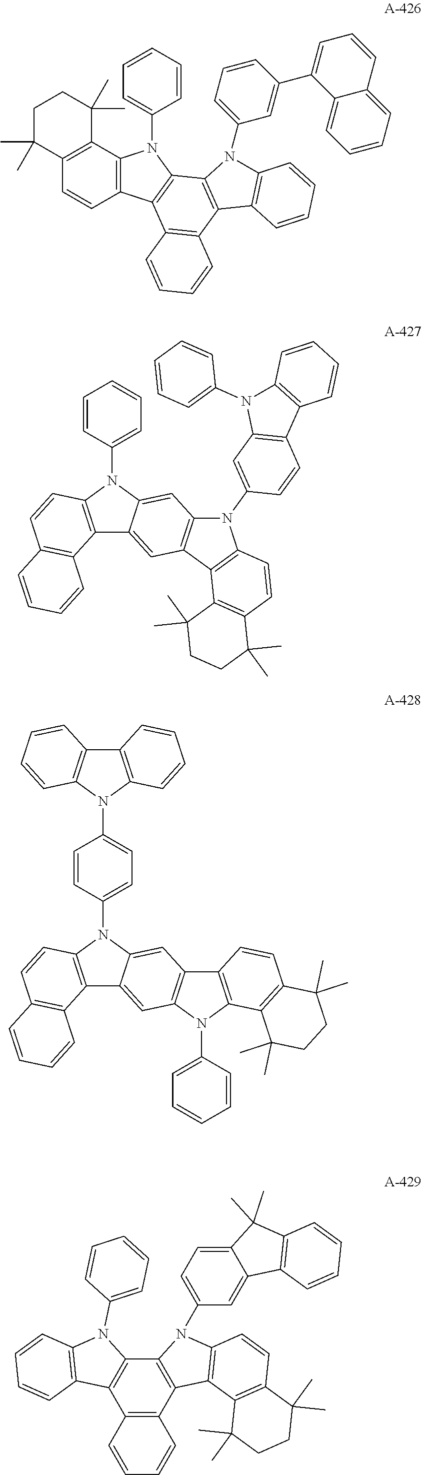 [SMILES: CC1(C)CCC(C)(C)c2c1ccc1c3c4ccccc4c4c5ccccc5n(-c5cccc(-c6cccc7ccccc67)c5)c4c3n(-c3ccccc3)c21 ; CC1(C)CCC(C)(C)c2c1ccc1c2c2cc3c4c5ccccc5ccc4n(-c4ccccc4)c3cc2n1-c1ccc2c3ccccc3n(-c3ccccc3)c2c1 ; CC1(C)CCC(C)(C)c2c1ccc1c3cc4c(cc3n(-c3ccccc3)c21)c1c2ccccc2ccc1n4-c1ccc(-n2c3ccccc3c3ccccc32)cc1 ; CC1(C)CCC(C)(C)c2c1ccc1c2c2c3ccccc3c3c4ccccc4n(-c4ccccc4)c3c2n1-c1ccc2c(c1)-c1ccccc1C2(C)C]

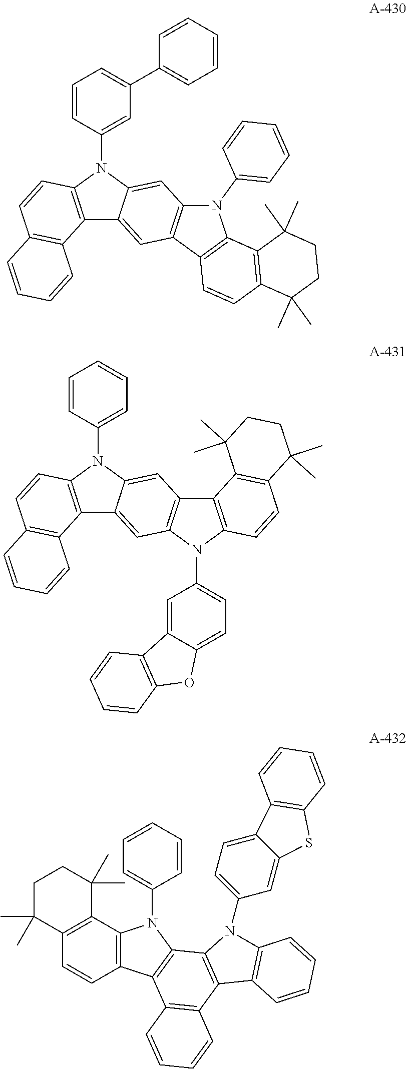

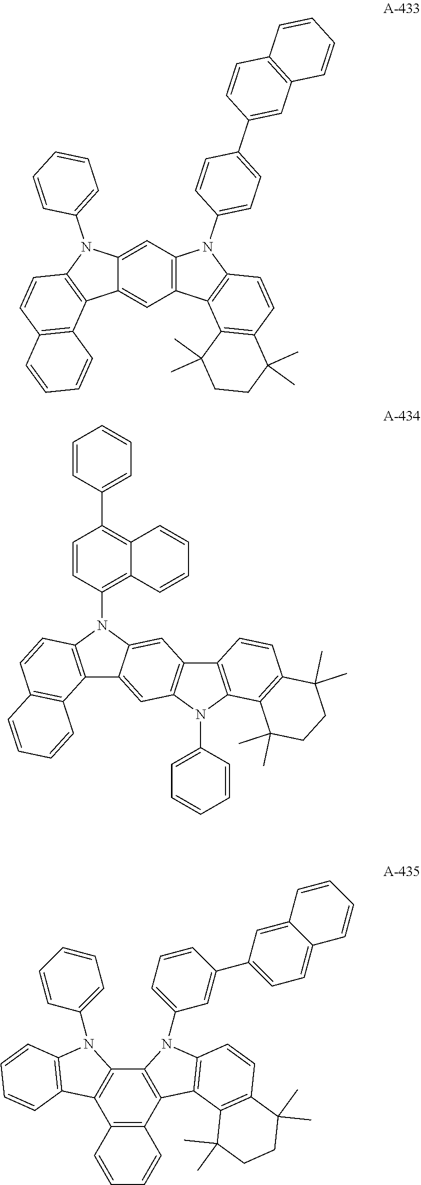

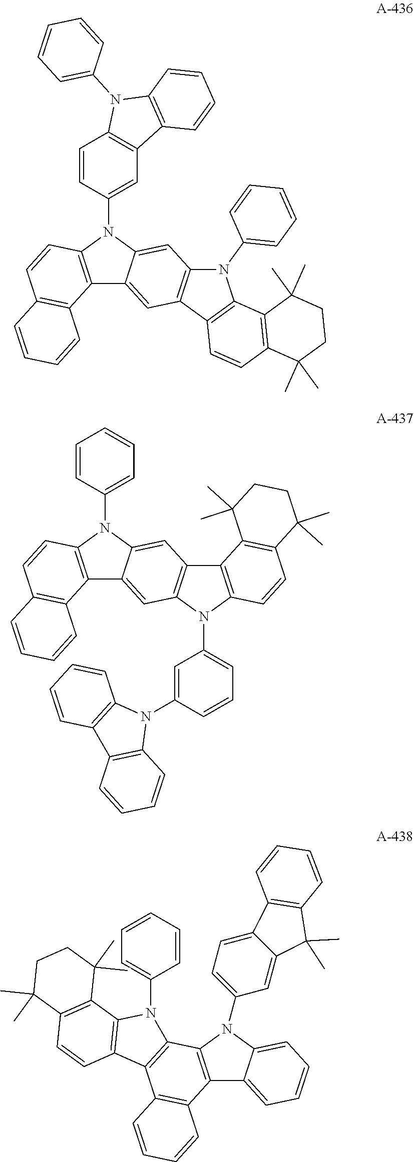

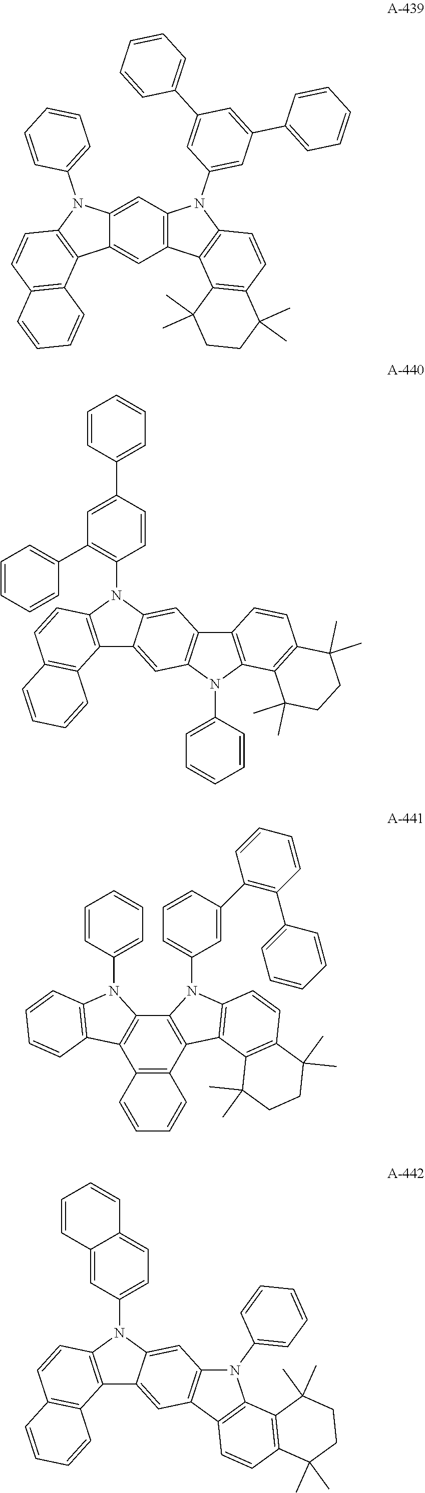 [SMILES: CC1(C)CCC(C)(C)c2c1ccc1c2c2cc3c4c5ccccc5ccc4n(-c4ccccc4)c3cc2n1-c1cc(-c2ccccc2)cc(-c2ccccc2)c1 ; CC1(C)CCC(C)(C)c2c1ccc1c3cc4c(cc3n(-c3ccccc3)c21)c1c2ccccc2ccc1n4-c1ccc(-c2ccccc2)cc1-c1ccccc1 ; CC1(C)CCC(C)(C)c2c1ccc1c2c2c3ccccc3c3c4ccccc4n(-c4ccccc4)c3c2n1-c1cccc(-c2ccccc2-c2ccccc2)c1 ; CC1(C)CCC(C)(C)c2c1ccc1c3cc4c5c6ccccc6ccc5n(-c5ccc6ccccc6c5)c4cc3n(-c3ccccc3)c21]

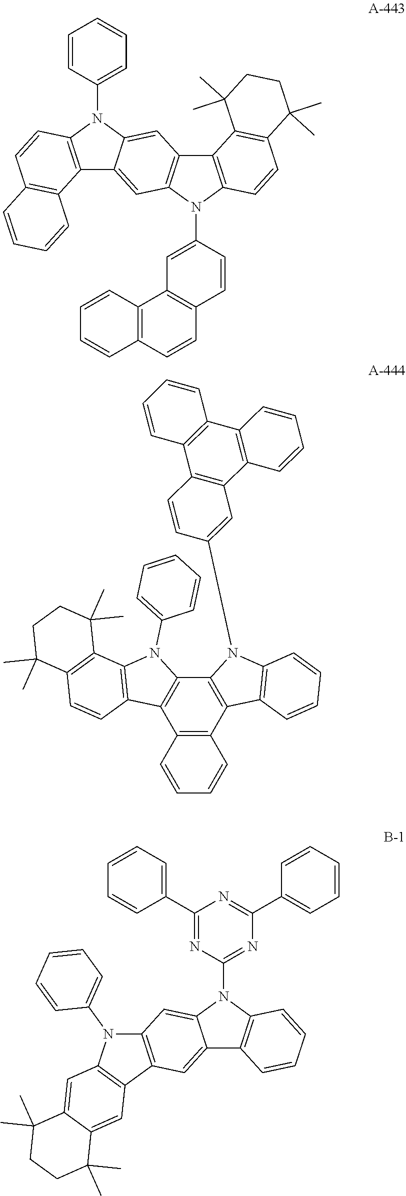

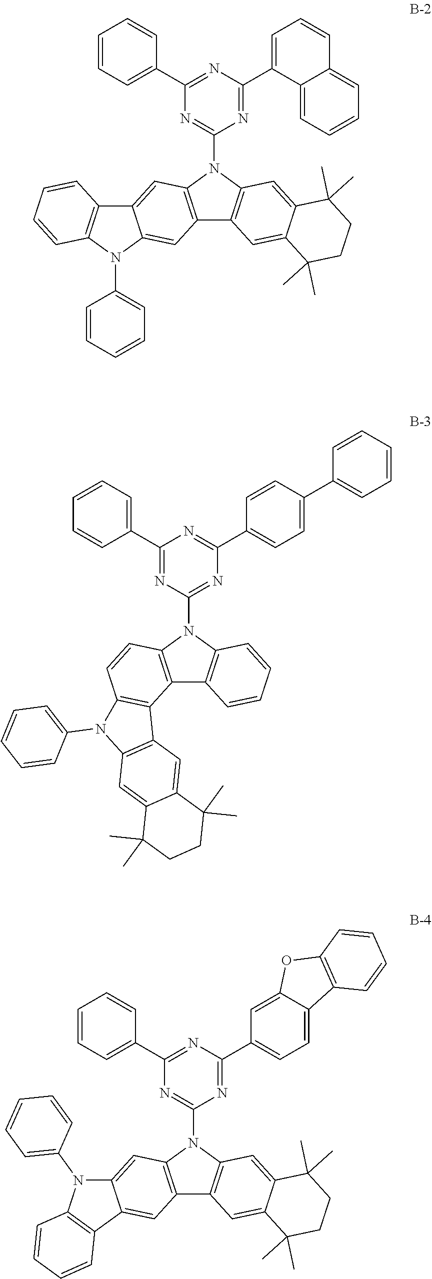 [SMILES: CC1(C)CCC(C)(C)c2cc3c(cc21)c1cc2c(cc1n3-c1nc(-c3ccccc3)nc(-c3cccc4ccccc34)n1)c1ccccc1n2-c1ccccc1 ; CC1(C)CCC(C)(C)c2cc3c(cc21)c1c2c4ccccc4n(-c4nc(-c5ccccc5)nc(-c5ccc(-c6ccccc6)cc5)n4)c2ccc1n3-c1ccccc1 ; CC1(C)CCC(C)(C)c2cc3c(cc21)c1cc2c4ccccc4n(-c4ccccc4)c2cc1n3-c1nc(-c2ccccc2)nc(-c2ccc3c(c2)oc2ccccc23)n1]

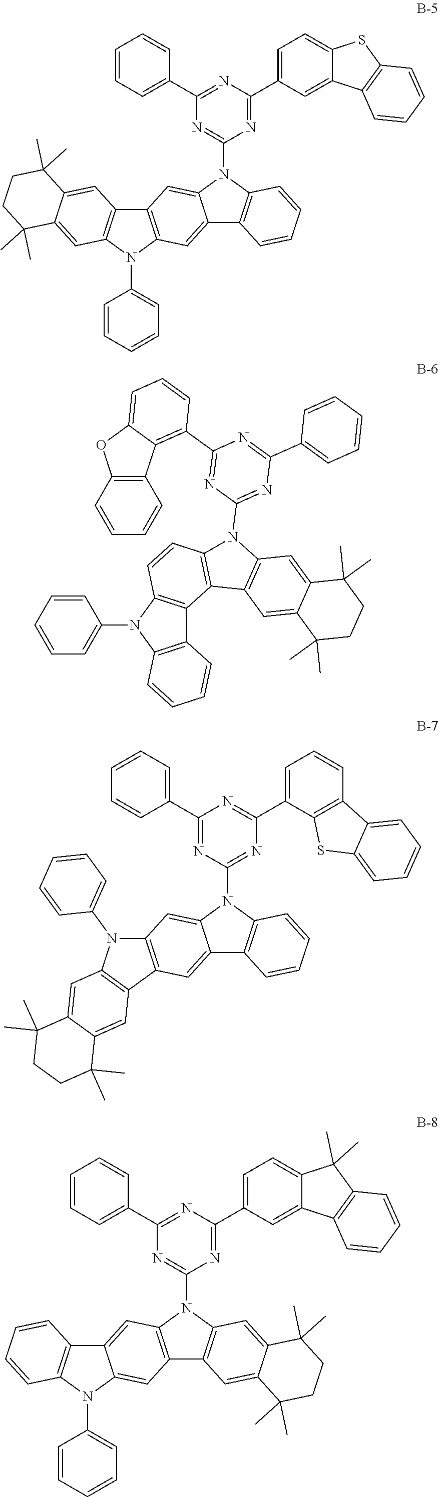

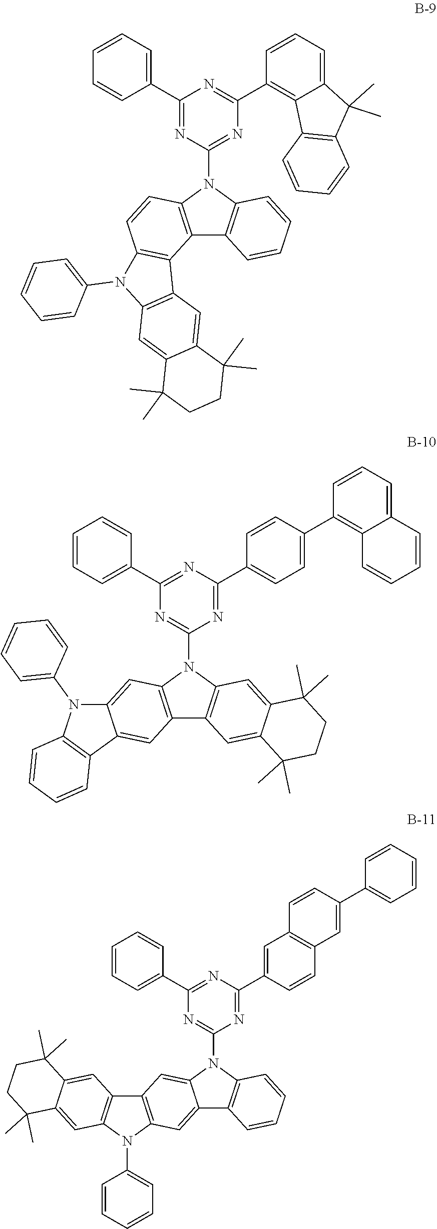

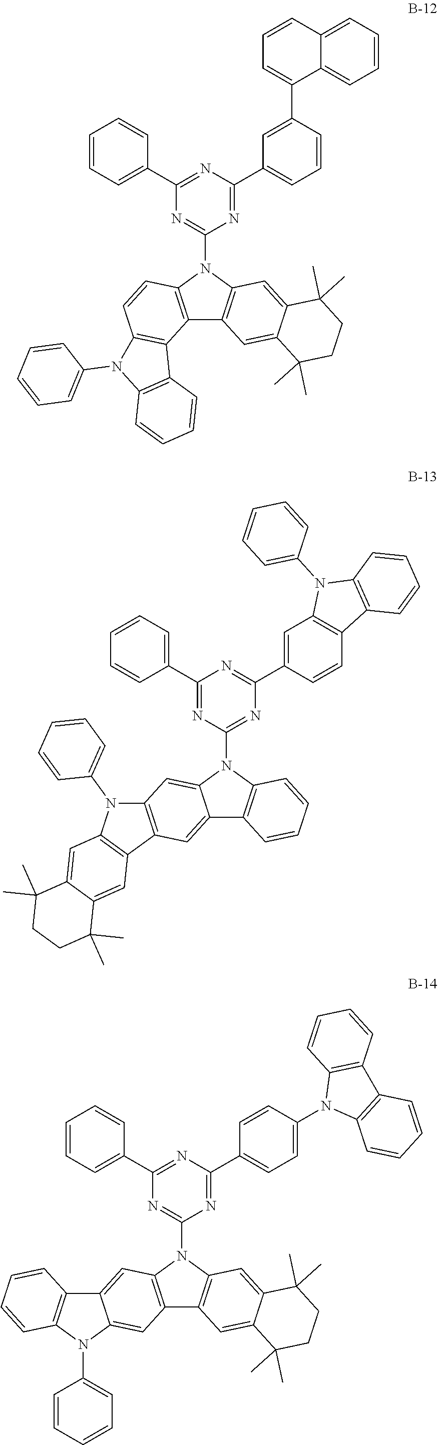 [SMILES: CC1(C)CCC(C)(C)c2cc3c(cc21)c1c2c4ccccc4n(-c4ccccc4)c2ccc1n3-c1nc(-c2ccccc2)nc(-c2cccc(-c3cccc4ccccc34)c2)n1 ; CC1(C)CCC(C)(C)c2cc3c(cc21)c1cc2c4ccccc4n(-c4nc(-c5ccccc5)nc(-c5ccc6c7ccccc7n(-c7ccccc7)c6c5)n4)c2cc1n3-c1ccccc1 ; CC1(C)CCC(C)(C)c2cc3c(cc21)c1cc2c(cc1n3-c1nc(-c3ccccc3)nc(-c3ccc(-n4c5ccccc5c5ccccc54)cc3)n1)c1ccccc1n2-c1ccccc1]

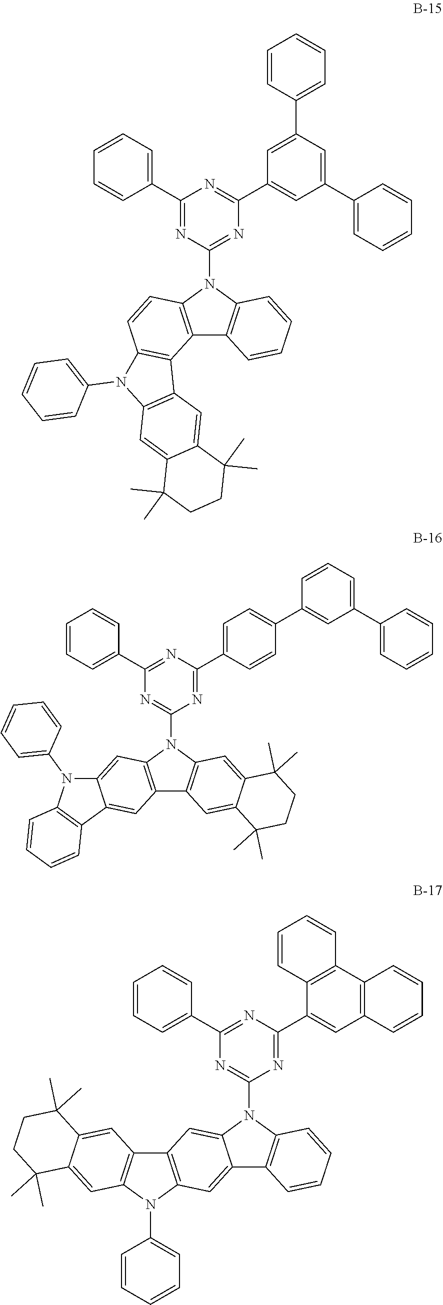 [SMILES: CC1(C)CCC(C)(C)c2cc3c(cc21)c1c2c4ccccc4n(-c4nc(-c5ccccc5)nc(-c5cc(-c6ccccc6)cc(-c6ccccc6)c5)n4)c2ccc1n3-c1ccccc1 ; CC1(C)CCC(C)(C)c2cc3c(cc21)c1cc2c4ccccc4n(-c4ccccc4)c2cc1n3-c1nc(-c2ccccc2)nc(-c2ccc(-c3cccc(-c4ccccc4)c3)cc2)n1 ; CC1(C)CCC(C)(C)c2cc3c(cc21)c1cc2c(cc1n3-c1ccccc1)c1ccccc1n2-c1nc(-c2ccccc2)nc(-c2cc3ccccc3c3ccccc23)n1]

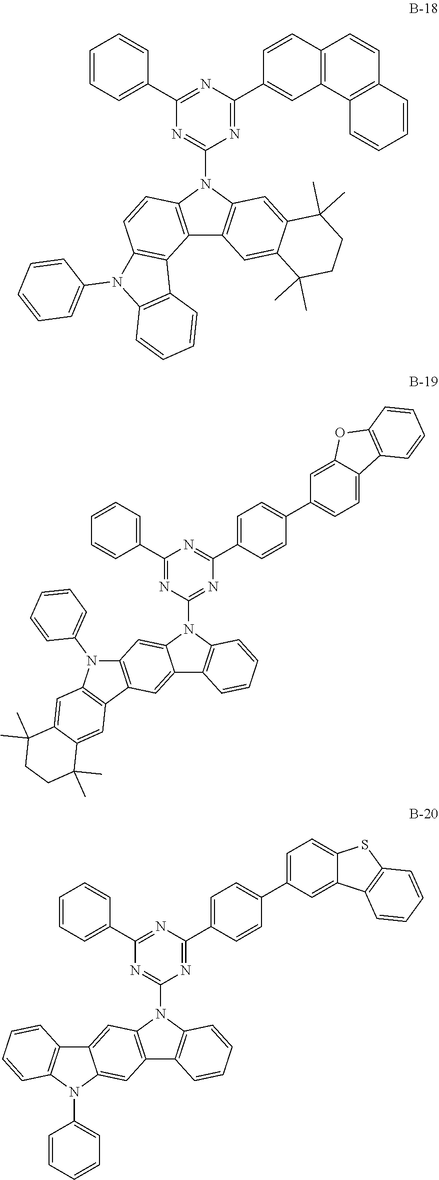 [SMILES: CC1(C)CCC(C)(C)c2cc3c(cc21)c1c2c4ccccc4n(-c4ccccc4)c2ccc1n3-c1nc(-c2ccccc2)nc(-c2ccc3ccc4ccccc4c3c2)n1 ; CC1(C)CCC(C)(C)c2cc3c(cc21)c1cc2c4ccccc4n(-c4nc(-c5ccccc5)nc(-c5ccc(-c6ccc7c(c6)oc6ccccc67)cc5)n4)c2cc1n3-c1ccccc1 ; c1ccc(-c2nc(-c3ccc(-c4ccc5sc6ccccc6c5c4)cc3)nc(-n3c4ccccc4c4cc5c(cc43)c3ccccc3n5-c3ccccc3)n2)cc1]

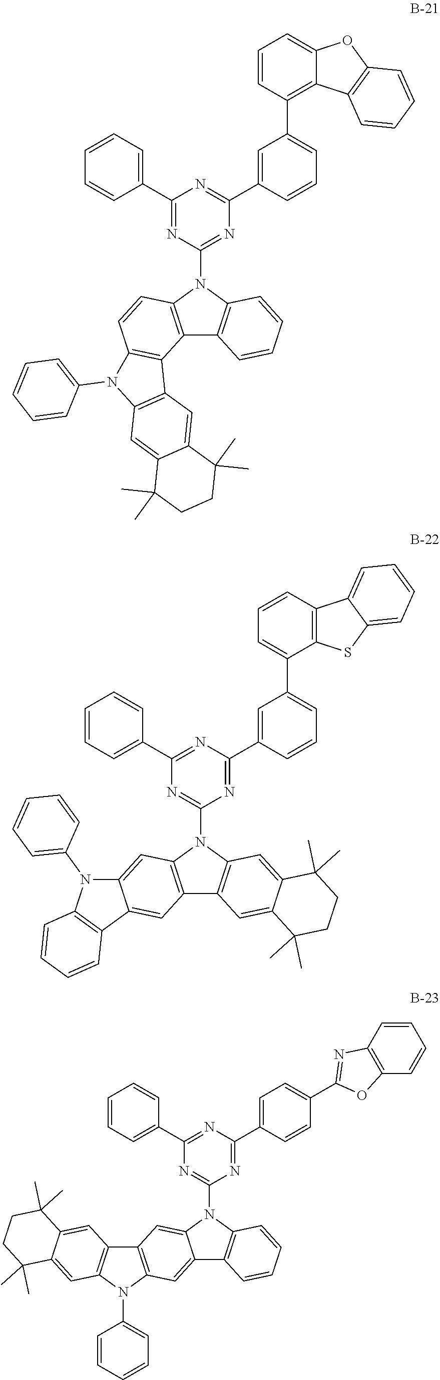 [SMILES: CC1(C)CCC(C)(C)c2cc3c(cc21)c1c2c4ccccc4n(-c4nc(-c5ccccc5)nc(-c5cccc(-c6cccc7oc8ccccc8c67)c5)n4)c2ccc1n3-c1ccccc1 ; CC1(C)CCC(C)(C)c2cc3c(cc21)c1cc2c4ccccc4n(-c4ccccc4)c2cc1n3-c1nc(-c2ccccc2)nc(-c2cccc(-c3cccc4c3sc3ccccc34)c2)n1 ; CC1(C)CCC(C)(C)c2cc3c(cc21)c1cc2c(cc1n3-c1ccccc1)c1ccccc1n2-c1nc(-c2ccccc2)nc(-c2ccc(-c3nc4ccccc4o3)cc2)n1]

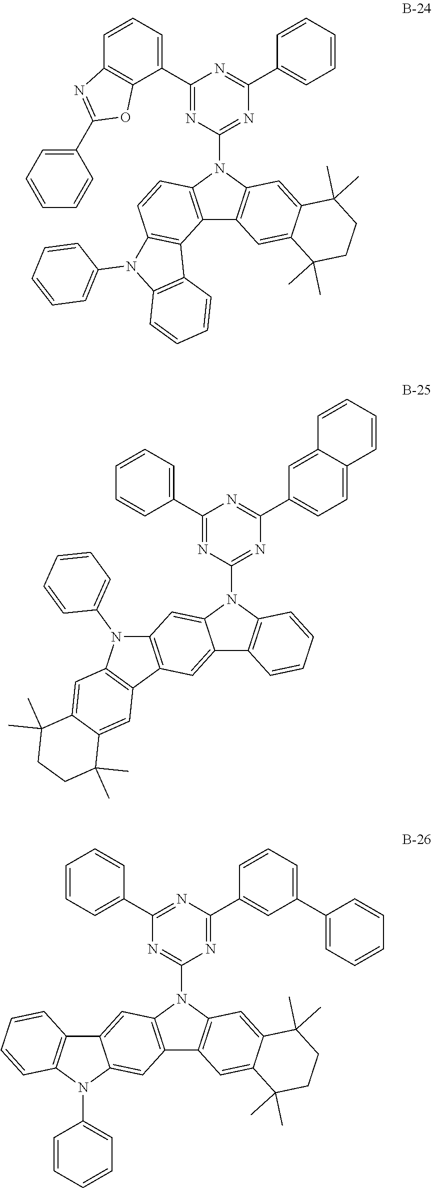

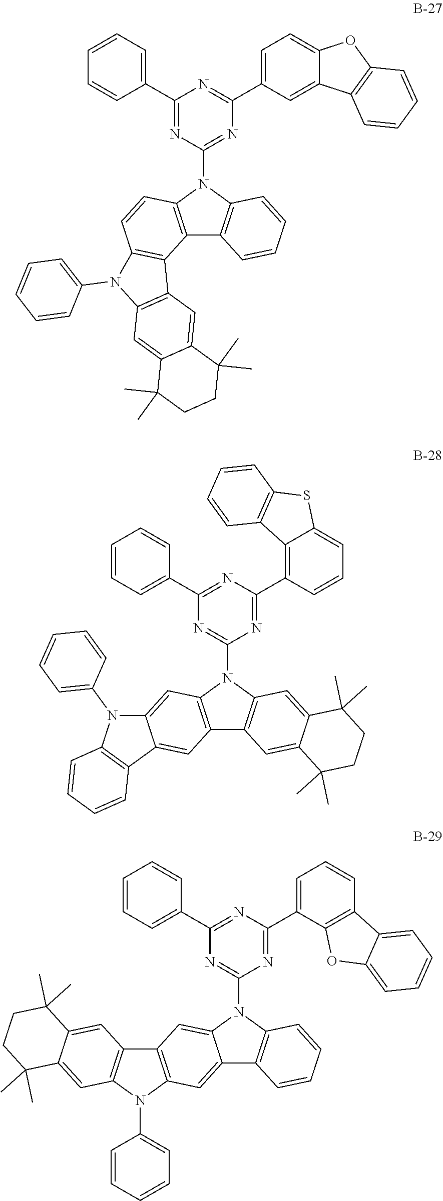

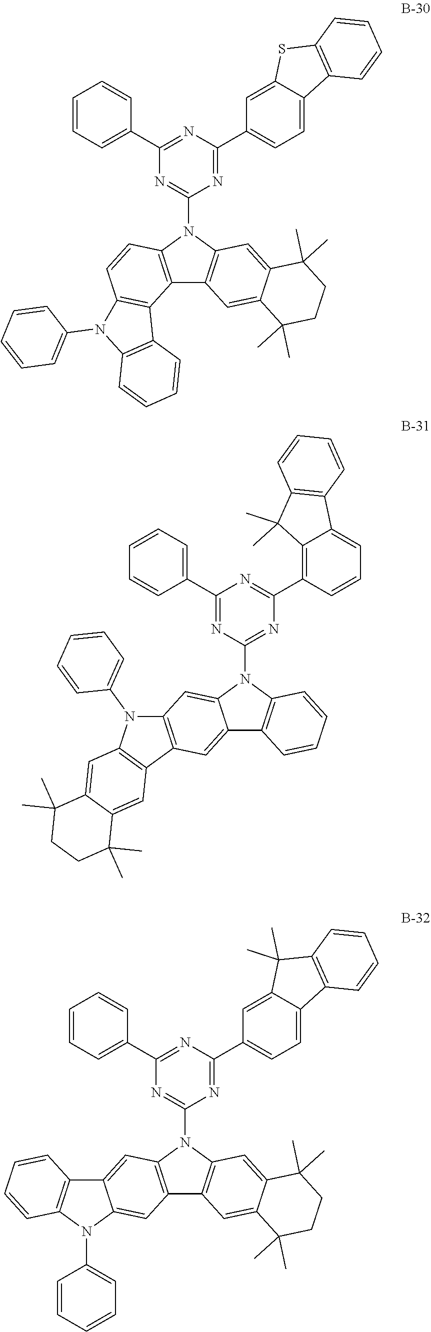 [SMILES: CC1(C)CCC(C)(C)c2cc3c(cc21)c1c2c4ccccc4n(-c4ccccc4)c2ccc1n3-c1nc(-c2ccccc2)nc(-c2ccc3c(c2)sc2ccccc23)n1 ; CC1(C)CCC(C)(C)c2cc3c(cc21)c1cc2c4ccccc4n(-c4nc(-c5ccccc5)nc(-c5cccc6c5C(C)(C)c5ccccc5-6)n4)c2cc1n3-c1ccccc1 ; CC1(C)CCC(C)(C)c2cc3c(cc21)c1cc2c(cc1n3-c1nc(-c3ccccc3)nc(-c3ccc4c(c3)C(C)(C)c3ccccc3-4)n1)c1ccccc1n2-c1ccccc1]

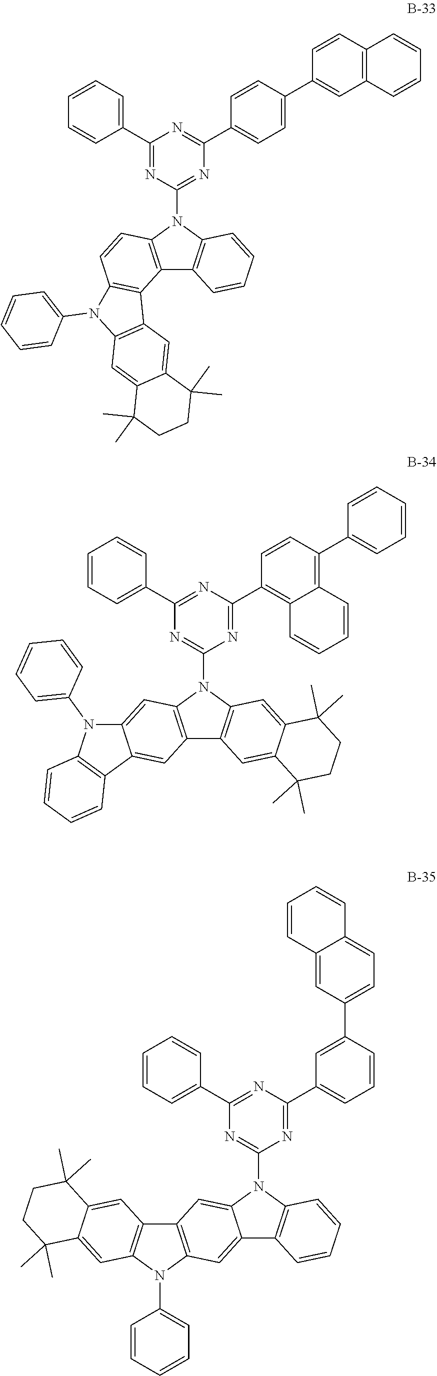 [SMILES: CC1(C)CCC(C)(C)c2cc3c(cc21)c1c2c4ccccc4n(-c4nc(-c5ccccc5)nc(-c5ccc(-c6ccc7ccccc7c6)cc5)n4)c2ccc1n3-c1ccccc1 ; CC1(C)CCC(C)(C)c2cc3c(cc21)c1cc2c4ccccc4n(-c4ccccc4)c2cc1n3-c1nc(-c2ccccc2)nc(-c2ccc(-c3ccccc3)c3ccccc23)n1 ; CC1(C)CCC(C)(C)c2cc3c(cc21)c1cc2c(cc1n3-c1ccccc1)c1ccccc1n2-c1nc(-c2ccccc2)nc(-c2cccc(-c3ccc4ccccc4c3)c2)n1]

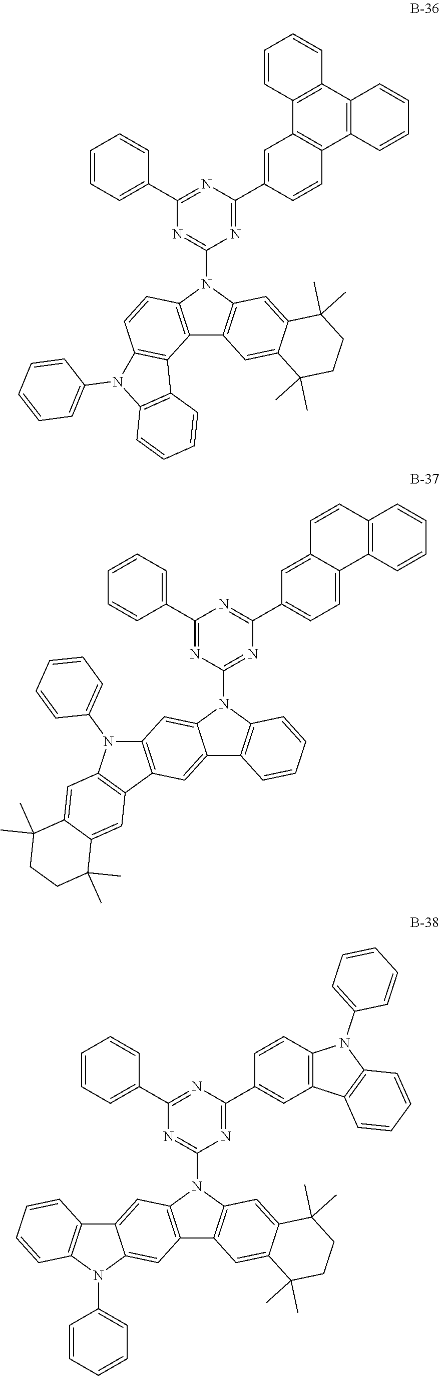 [SMILES: CC1(C)CCC(C)(C)c2cc3c(cc21)c1c2c4ccccc4n(-c4ccccc4)c2ccc1n3-c1nc(-c2ccccc2)nc(-c2ccc3c4ccccc4c4ccccc4c3c2)n1 ; CC1(C)CCC(C)(C)c2cc3c(cc21)c1cc2c4ccccc4n(-c4nc(-c5ccccc5)nc(-c5ccc6c(ccc7ccccc76)c5)n4)c2cc1n3-c1ccccc1 ; CC1(C)CCC(C)(C)c2cc3c(cc21)c1cc2c(cc1n3-c1nc(-c3ccccc3)nc(-c3ccc4c(c3)c3ccccc3n4-c3ccccc3)n1)c1ccccc1n2-c1ccccc1]

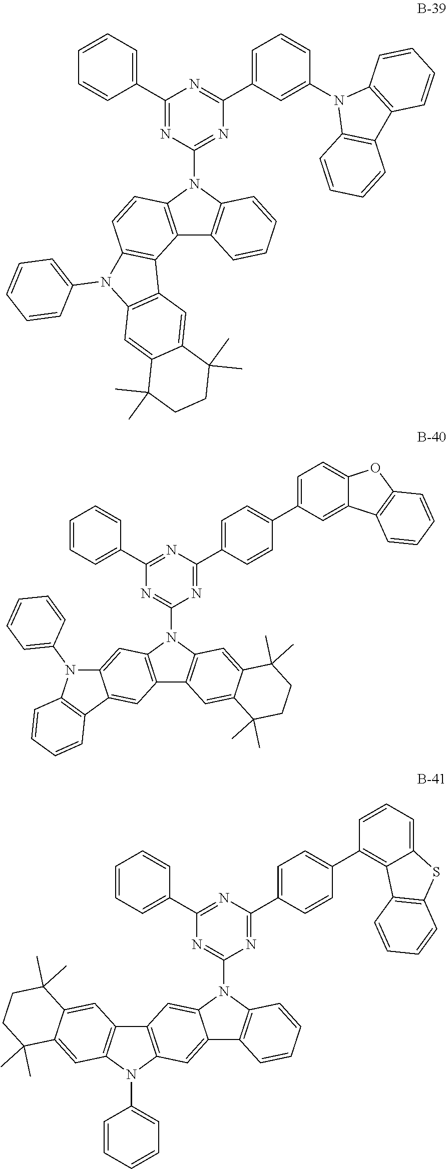 [SMILES: CC1(C)CCC(C)(C)c2cc3c(cc21)c1c2c4ccccc4n(-c4nc(-c5ccccc5)nc(-c5cccc(-n6c7ccccc7c7ccccc76)c5)n4)c2ccc1n3-c1ccccc1 ; CC1(C)CCC(C)(C)c2cc3c(cc21)c1cc2c4ccccc4n(-c4ccccc4)c2cc1n3-c1nc(-c2ccccc2)nc(-c2ccc(-c3ccc4oc5ccccc5c4c3)cc2)n1 ; CC1(C)CCC(C)(C)c2cc3c(cc21)c1cc2c(cc1n3-c1ccccc1)c1ccccc1n2-c1nc(-c2ccccc2)nc(-c2ccc(-c3cccc4sc5ccccc5c34)cc2)n1]

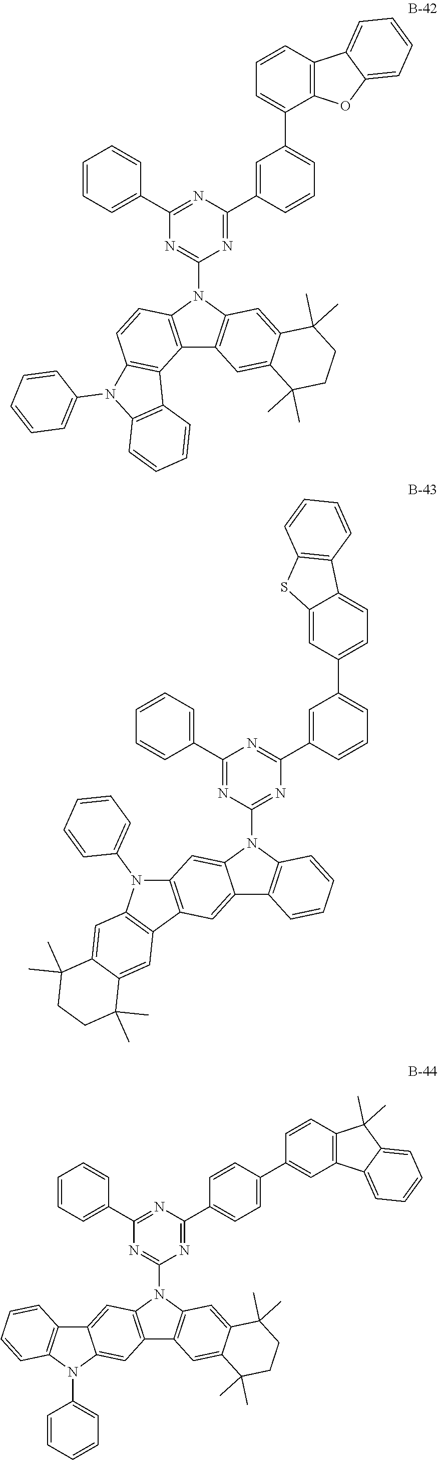 [SMILES: CC1(C)CCC(C)(C)c2cc3c(cc21)c1c2c4ccccc4n(-c4ccccc4)c2ccc1n3-c1nc(-c2ccccc2)nc(-c2cccc(-c3cccc4c3oc3ccccc34)c2)n1 ; CC1(C)CCC(C)(C)c2cc3c(cc21)c1cc2c4ccccc4n(-c4nc(-c5ccccc5)nc(-c5cccc(-c6ccc7c(c6)sc6ccccc67)c5)n4)c2cc1n3-c1ccccc1 ; CC1(C)CCC(C)(C)c2cc3c(cc21)c1cc2c(cc1n3-c1nc(-c3ccccc3)nc(-c3ccc(-c4ccc5c(c4)-c4ccccc4C5(C)C)cc3)n1)c1ccccc1n2-c1ccccc1]

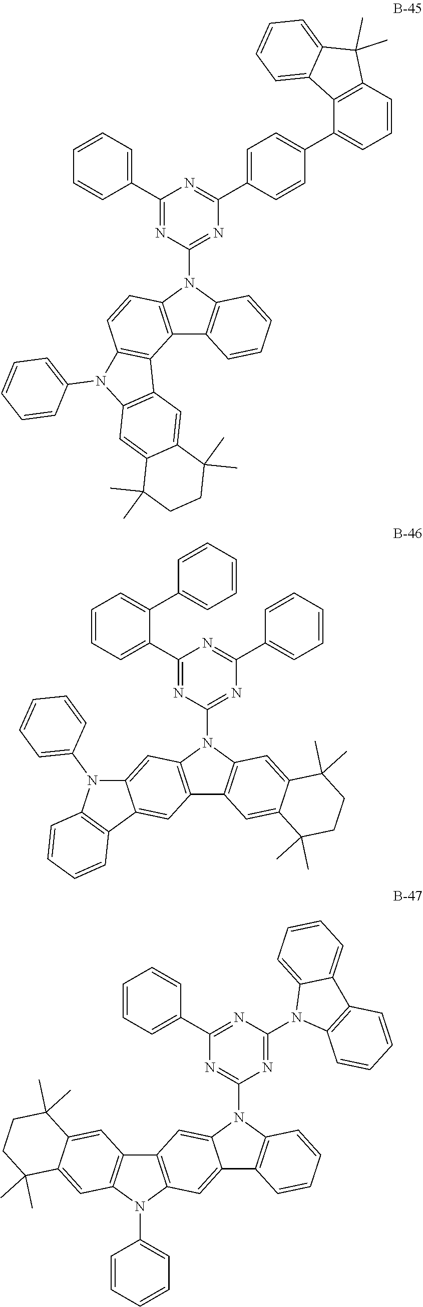

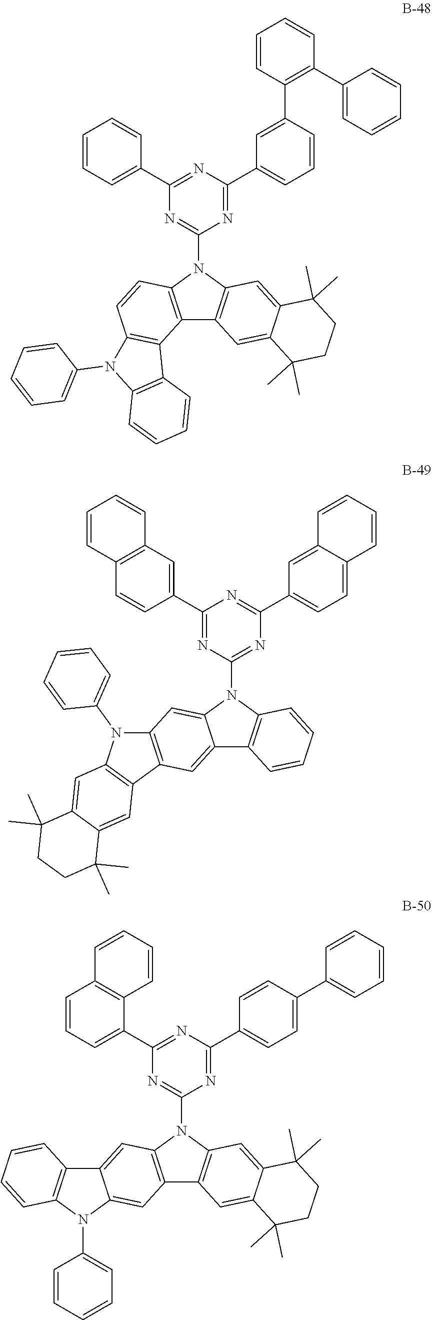

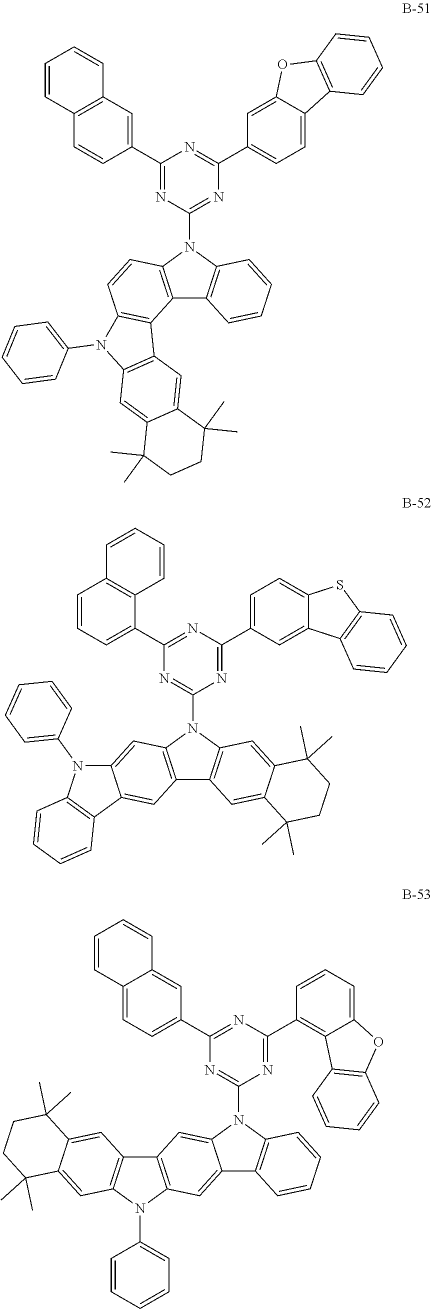 [SMILES: CC1(C)CCC(C)(C)c2cc3c(cc21)c1c2c4ccccc4n(-c4nc(-c5ccc6ccccc6c5)nc(-c5ccc6c(c5)oc5ccccc56)n4)c2ccc1n3-c1ccccc1 ; CC1(C)CCC(C)(C)c2cc3c(cc21)c1cc2c4ccccc4n(-c4ccccc4)c2cc1n3-c1nc(-c2ccc3sc4ccccc4c3c2)nc(-c2cccc3ccccc23)n1 ; CC1(C)CCC(C)(C)c2cc3c(cc21)c1cc2c(cc1n3-c1ccccc1)c1ccccc1n2-c1nc(-c2ccc3ccccc3c2)nc(-c2cccc3oc4ccccc4c23)n1]

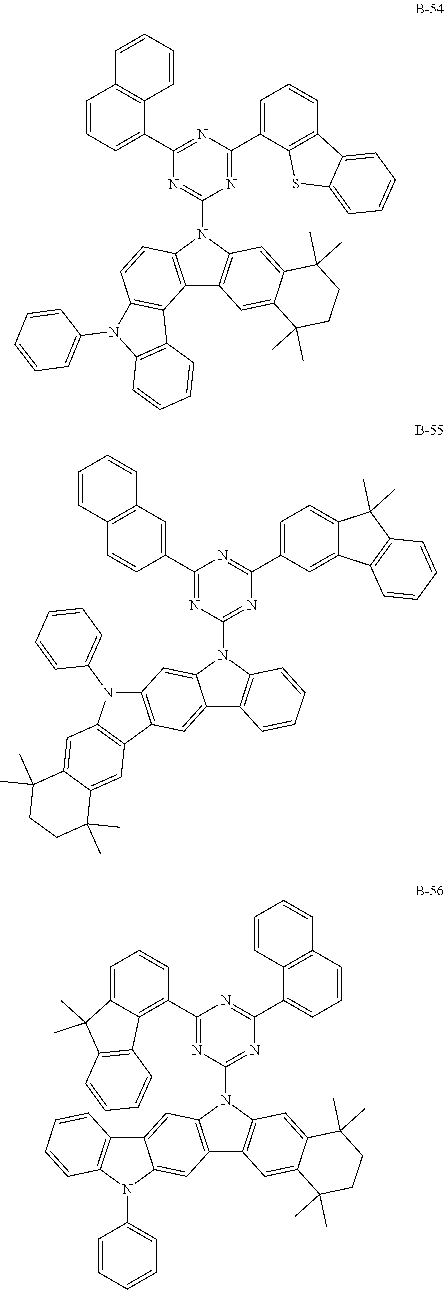 [SMILES: CC1(C)CCC(C)(C)c2cc3c(cc21)c1c2c4ccccc4n(-c4ccccc4)c2ccc1n3-c1nc(-c2cccc3ccccc23)nc(-c2cccc3c2sc2ccccc23)n1 ; CC1(C)CCC(C)(C)c2cc3c(cc21)c1cc2c4ccccc4n(-c4nc(-c5ccc6c(c5)-c5ccccc5C6(C)C)nc(-c5ccc6ccccc6c5)n4)c2cc1n3-c1ccccc1 ; CC1(C)CCC(C)(C)c2cc3c(cc21)c1cc2c(cc1n3-c1nc(-c3cccc4c3-c3ccccc3C4(C)C)nc(-c3cccc4ccccc34)n1)c1ccccc1n2-c1ccccc1]

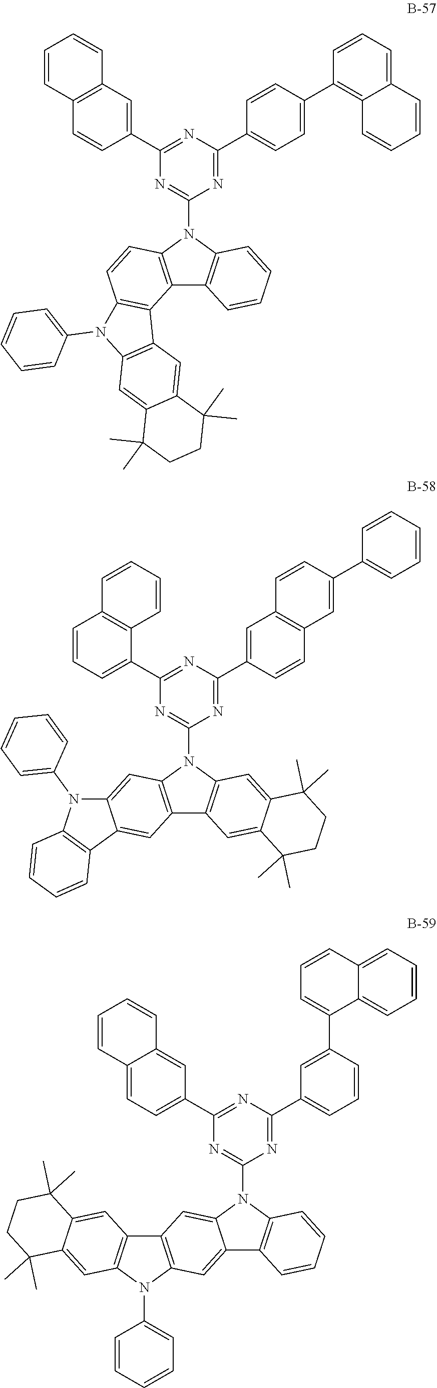 [SMILES: CC1(C)CCC(C)(C)c2cc3c(cc21)c1c2c4ccccc4n(-c4nc(-c5ccc(-c6cccc7ccccc67)cc5)nc(-c5ccc6ccccc6c5)n4)c2ccc1n3-c1ccccc1 ; CC1(C)CCC(C)(C)c2cc3c(cc21)c1cc2c4ccccc4n(-c4ccccc4)c2cc1n3-c1nc(-c2ccc3cc(-c4ccccc4)ccc3c2)nc(-c2cccc3ccccc23)n1 ; CC1(C)CCC(C)(C)c2cc3c(cc21)c1cc2c(cc1n3-c1ccccc1)c1ccccc1n2-c1nc(-c2cccc(-c3cccc4ccccc34)c2)nc(-c2ccc3ccccc3c2)n1]

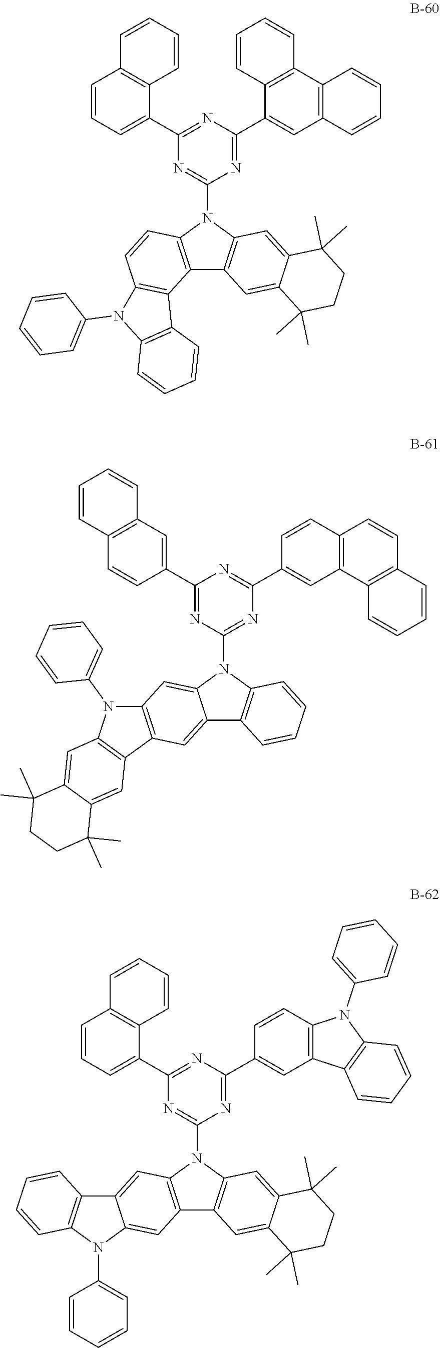 [SMILES: CC1(C)CCC(C)(C)c2cc3c(cc21)c1c2c4ccccc4n(-c4ccccc4)c2ccc1n3-c1nc(-c2cccc3ccccc23)nc(-c2cc3ccccc3c3ccccc23)n1 ; CC1(C)CCC(C)(C)c2cc3c(cc21)c1cc2c4ccccc4n(-c4nc(-c5ccc6ccccc6c5)nc(-c5ccc6ccc7ccccc7c6c5)n4)c2cc1n3-c1ccccc1 ; CC1(C)CCC(C)(C)c2cc3c(cc21)c1cc2c(cc1n3-c1nc(-c3ccc4c(c3)c3ccccc3n4-c3ccccc3)nc(-c3cccc4ccccc34)n1)c1ccccc1n2-c1ccccc1]

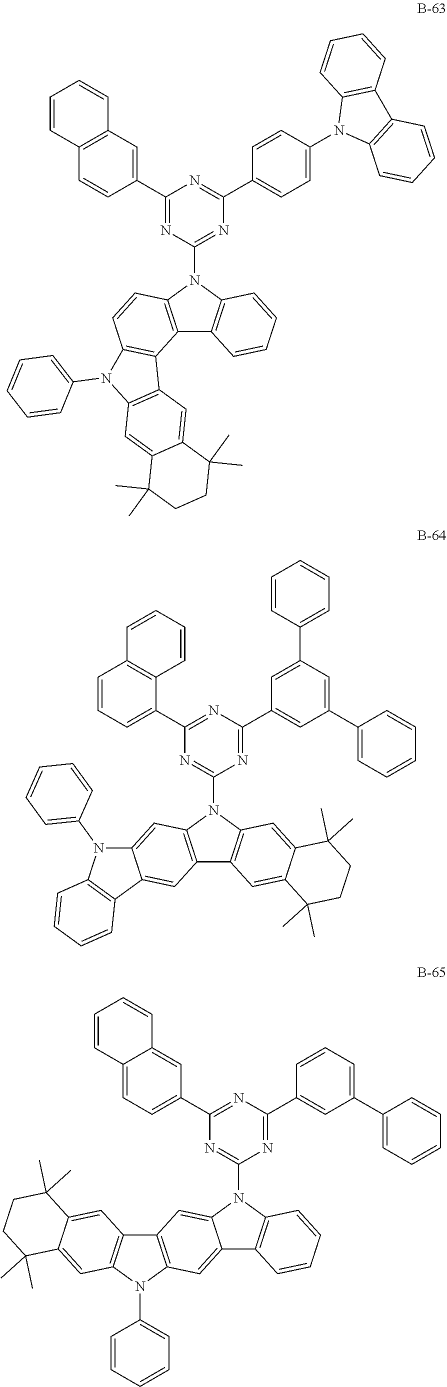 [SMILES: CC1(C)CCC(C)(C)c2cc3c(cc21)c1c2c4ccccc4n(-c4nc(-c5ccc(-n6c7ccccc7c7ccccc76)cc5)nc(-c5ccc6ccccc6c5)n4)c2ccc1n3-c1ccccc1 ; CC1(C)CCC(C)(C)c2cc3c(cc21)c1cc2c4ccccc4n(-c4ccccc4)c2cc1n3-c1nc(-c2cc(-c3ccccc3)cc(-c3ccccc3)c2)nc(-c2cccc3ccccc23)n1 ; CC1(C)CCC(C)(C)c2cc3c(cc21)c1cc2c(cc1n3-c1ccccc1)c1ccccc1n2-c1nc(-c2cccc(-c3ccccc3)c2)nc(-c2ccc3ccccc3c2)n1]

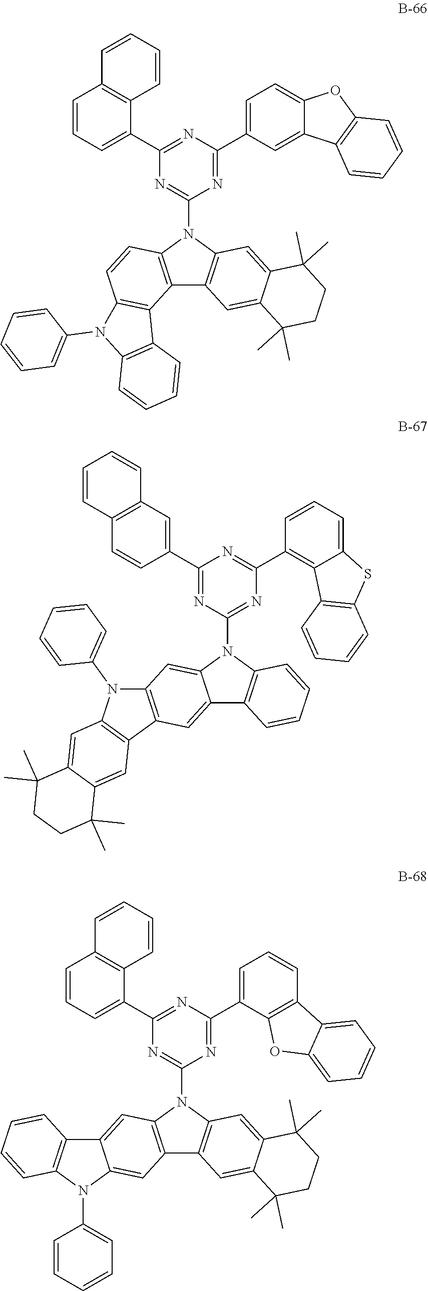 [SMILES: CC1(C)CCC(C)(C)c2cc3c(cc21)c1c2c4ccccc4n(-c4ccccc4)c2ccc1n3-c1nc(-c2ccc3oc4ccccc4c3c2)nc(-c2cccc3ccccc23)n1 ; CC1(C)CCC(C)(C)c2cc3c(cc21)c1cc2c4ccccc4n(-c4nc(-c5ccc6ccccc6c5)nc(-c5cccc6sc7ccccc7c56)n4)c2cc1n3-c1ccccc1 ; CC1(C)CCC(C)(C)c2cc3c(cc21)c1cc2c(cc1n3-c1nc(-c3cccc4ccccc34)nc(-c3cccc4c3oc3ccccc34)n1)c1ccccc1n2-c1ccccc1]

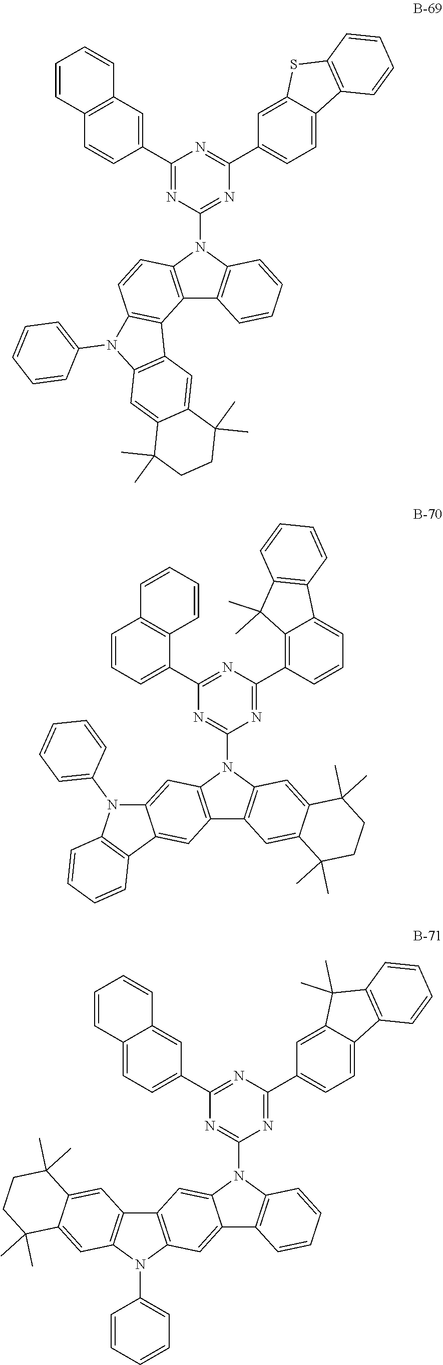

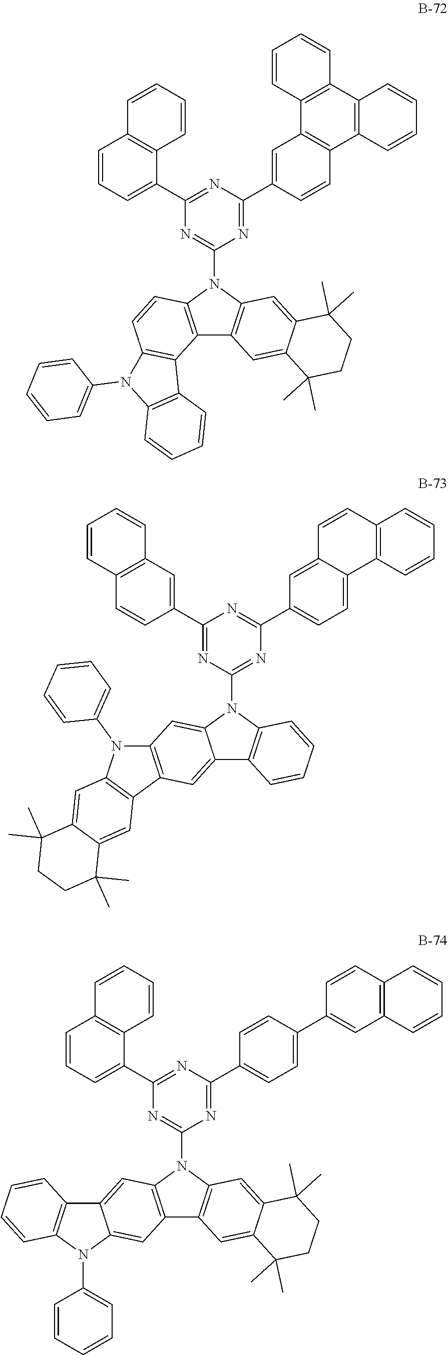 [SMILES: CC1(C)CCC(C)(C)c2cc3c(cc21)c1c2c4ccccc4n(-c4ccccc4)c2ccc1n3-c1nc(-c2ccc3c4ccccc4c4ccccc4c3c2)nc(-c2cccc3ccccc23)n1 ; CC1(C)CCC(C)(C)c2cc3c(cc21)c1cc2c4ccccc4n(-c4nc(-c5ccc6ccccc6c5)nc(-c5ccc6c(ccc7ccccc76)c5)n4)c2cc1n3-c1ccccc1 ; CC1(C)CCC(C)(C)c2cc3c(cc21)c1cc2c(cc1n3-c1nc(-c3ccc(-c4ccc5ccccc5c4)cc3)nc(-c3cccc4ccccc34)n1)c1ccccc1n2-c1ccccc1]

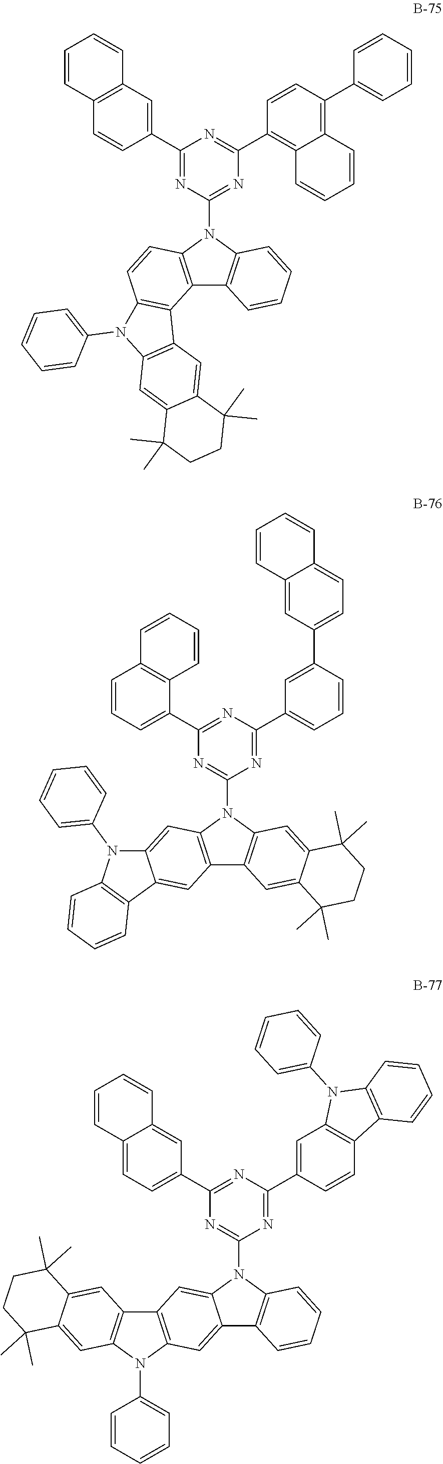

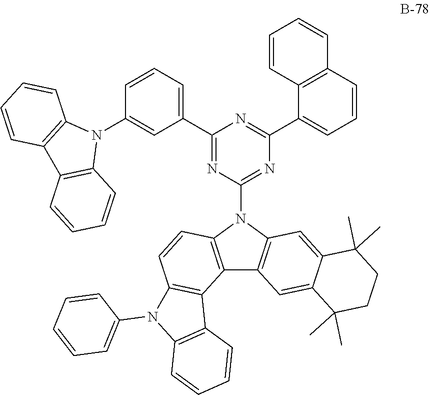 [SMILES: CC1(C)CCC(C)(C)c2cc3c(cc21)c1c2c4ccccc4n(-c4ccccc4)c2ccc1n3-c1nc(-c2cccc(-n3c4ccccc4c4ccccc43)c2)nc(-c2cccc3ccccc23)n1]

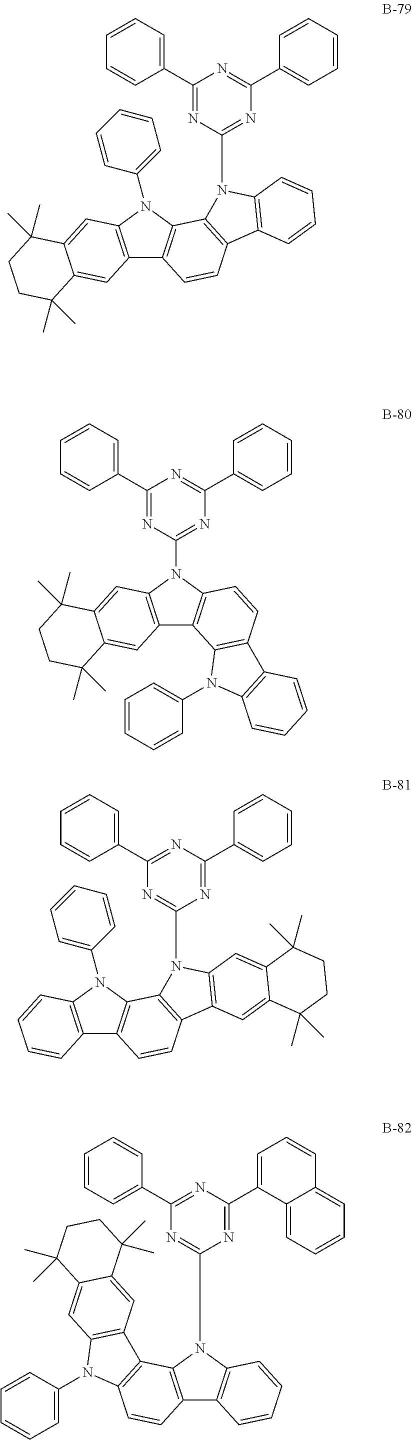 [SMILES: CC1(C)CCC(C)(C)c2cc3c(cc21)c1ccc2c4ccccc4n(-c4nc(-c5ccccc5)nc(-c5ccccc5)n4)c2c1n3-c1ccccc1 ; CC1(C)CCC(C)(C)c2cc3c(cc21)c1c(ccc2c4ccccc4n(-c4ccccc4)c21)n3-c1nc(-c2ccccc2)nc(-c2ccccc2)n1 ; CC1(C)CCC(C)(C)c2cc3c(cc21)c1ccc2c4ccccc4n(-c4ccccc4)c2c1n3-c1nc(-c2ccccc2)nc(-c2ccccc2)n1 ; CC1(C)CCC(C)(C)c2cc3c(cc21)c1c(ccc2c4ccccc4n(-c4nc(-c5ccccc5)nc(-c5cccc6ccccc56)n4)c21)n3-c1ccccc1]

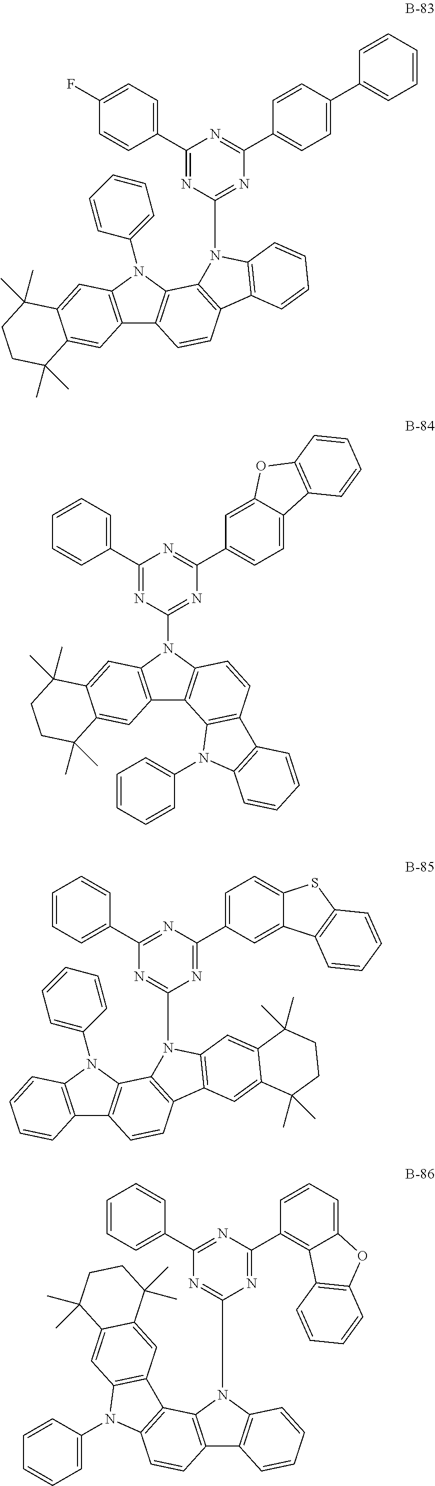

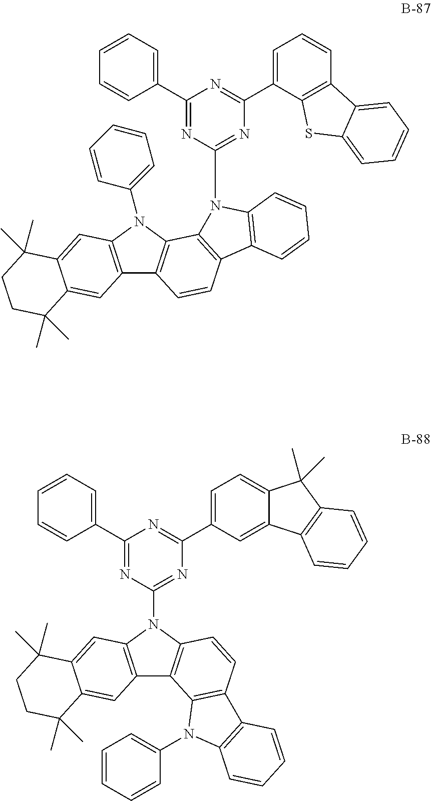 [SMILES: CC1(C)CCC(C)(C)c2cc3c(cc21)c1ccc2c4ccccc4n(-c4nc(-c5ccccc5)nc(-c5cccc6c5sc5ccccc56)n4)c2c1n3-c1ccccc1 ; CC1(C)CCC(C)(C)c2cc3c(cc21)c1c(ccc2c4ccccc4n(-c4ccccc4)c21)n3-c1nc(-c2ccccc2)nc(-c2ccc3c(c2)-c2ccccc2C3(C)C)n1]

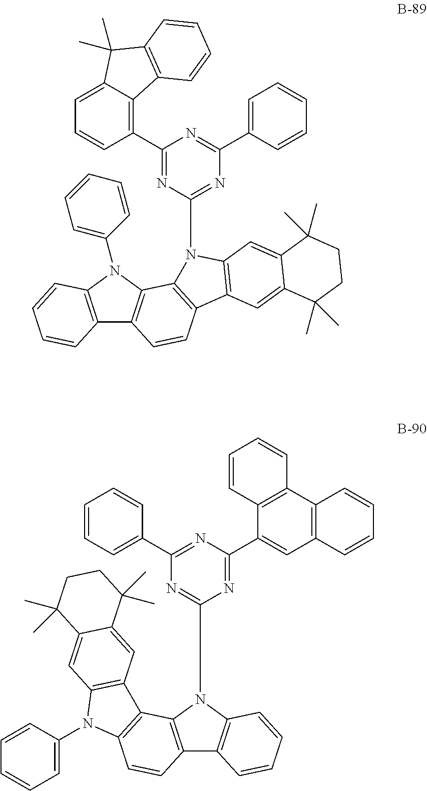 [SMILES: CC1(C)CCC(C)(C)c2cc3c(cc21)c1ccc2c4ccccc4n(-c4ccccc4)c2c1n3-c1nc(-c2ccccc2)nc(-c2cccc3c2-c2ccccc2C3(C)C)n1 ; CC1(C)CCC(C)(C)c2cc3c(cc21)c1c(ccc2c4ccccc4n(-c4nc(-c5ccccc5)nc(-c5cc6ccccc6c6ccccc56)n4)c21)n3-c1ccccc1]

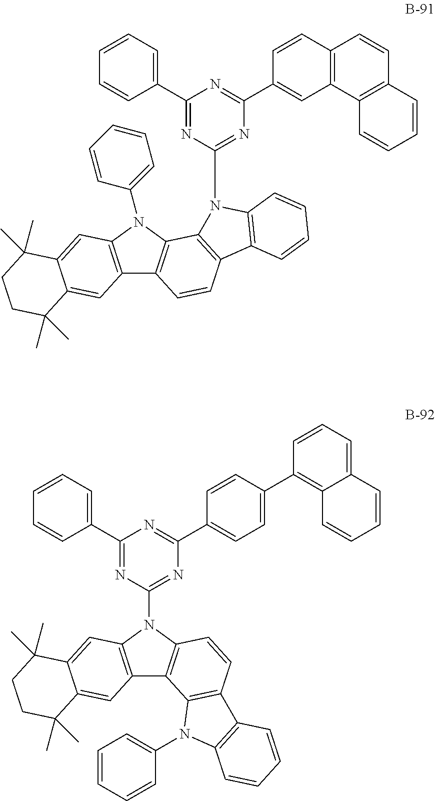 [SMILES: CC1(C)CCC(C)(C)c2cc3c(cc21)c1ccc2c4ccccc4n(-c4nc(-c5ccccc5)nc(-c5ccc6ccc7ccccc7c6c5)n4)c2c1n3-c1ccccc1 ; CC1(C)CCC(C)(C)c2cc3c(cc21)c1c(ccc2c4ccccc4n(-c4ccccc4)c21)n3-c1nc(-c2ccccc2)nc(-c2ccc(-c3cccc4ccccc34)cc2)n1]

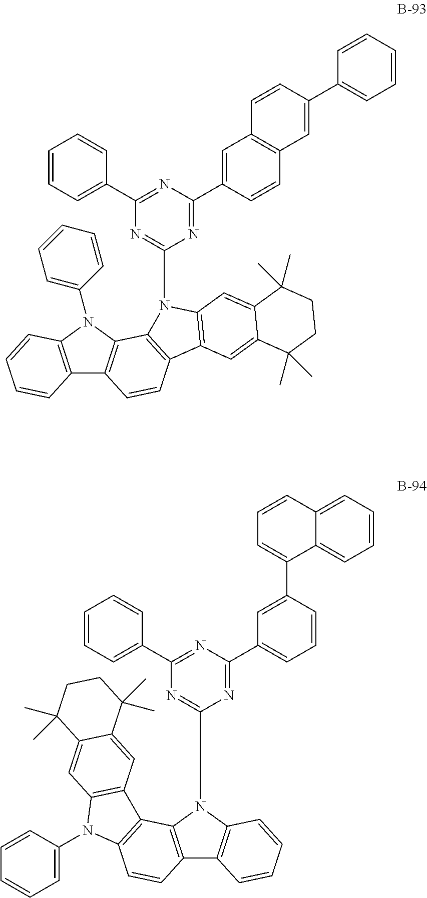 [SMILES: CC1(C)CCC(C)(C)c2cc3c(cc21)c1ccc2c4ccccc4n(-c4ccccc4)c2c1n3-c1nc(-c2ccccc2)nc(-c2ccc3cc(-c4ccccc4)ccc3c2)n1 ; CC1(C)CCC(C)(C)c2cc3c(cc21)c1c(ccc2c4ccccc4n(-c4nc(-c5ccccc5)nc(-c5cccc(-c6cccc7ccccc67)c5)n4)c21)n3-c1ccccc1]

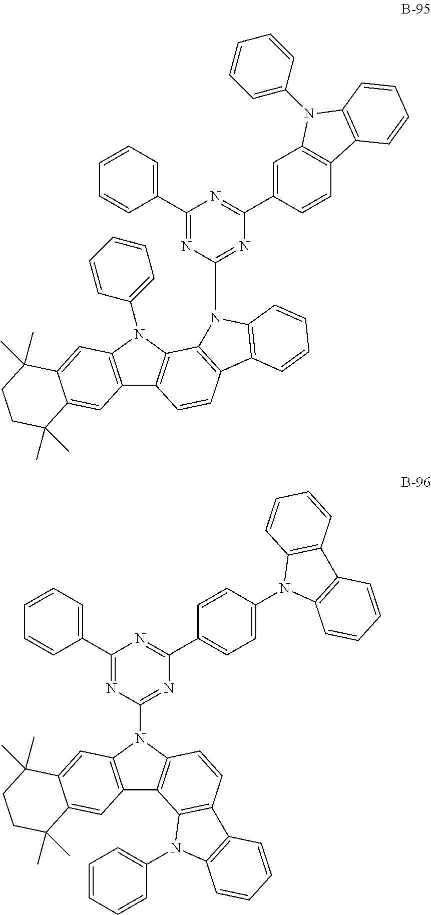 [SMILES: CC1(C)CCC(C)(C)c2cc3c(cc21)c1ccc2c4ccccc4n(-c4nc(-c5ccccc5)nc(-c5ccc6c7ccccc7n(-c7ccccc7)c6c5)n4)c2c1n3-c1ccccc1 ; CC1(C)CCC(C)(C)c2cc3c(cc21)c1c(ccc2c4ccccc4n(-c4ccccc4)c21)n3-c1nc(-c2ccccc2)nc(-c2ccc(-n3c4ccccc4c4ccccc43)cc2)n1]

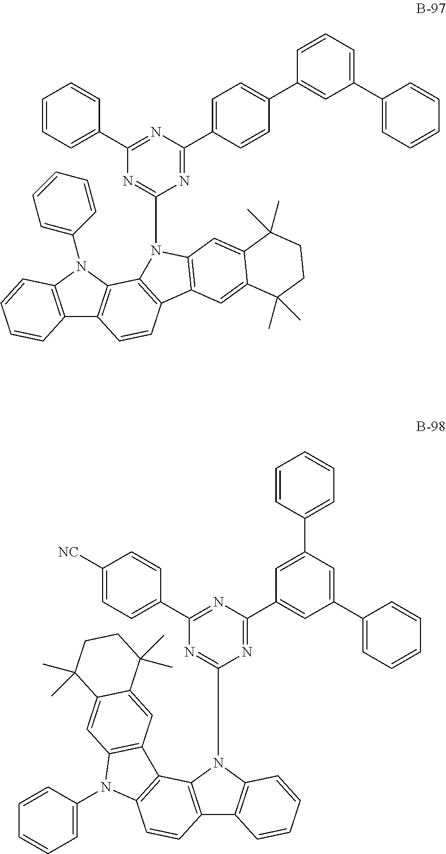 [SMILES: CC1(C)CCC(C)(C)c2cc3c(cc21)c1ccc2c4ccccc4n(-c4ccccc4)c2c1n3-c1nc(-c2ccccc2)nc(-c2ccc(-c3cccc(-c4ccccc4)c3)cc2)n1 ; CC1(C)CCC(C)(C)c2cc3c(cc21)c1c(ccc2c4ccccc4n(-c4nc(-c5ccc(C#N)cc5)nc(-c5cc(-c6ccccc6)cc(-c6ccccc6)c5)n4)c21)n3-c1ccccc1]

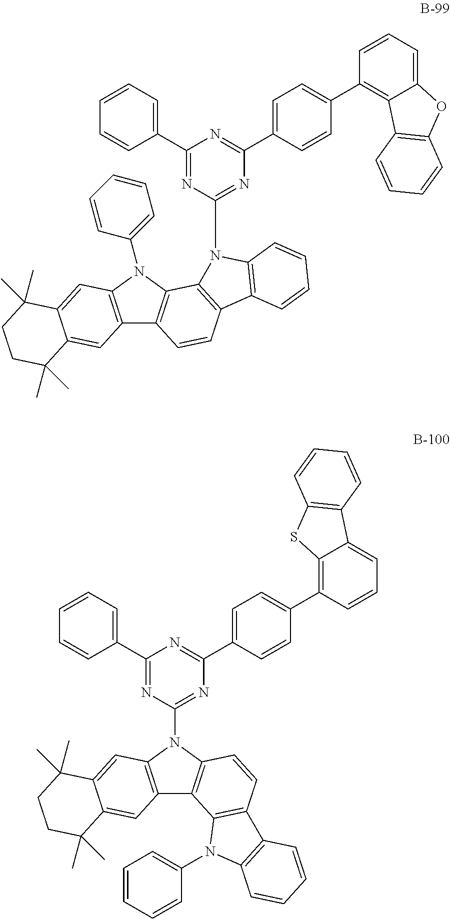 [SMILES: CC1(C)CCC(C)(C)c2cc3c(cc21)c1ccc2c4ccccc4n(-c4nc(-c5ccccc5)nc(-c5ccc(-c6cccc7oc8ccccc8c67)cc5)n4)c2c1n3-c1ccccc1 ; CC1(C)CCC(C)(C)c2cc3c(cc21)c1c(ccc2c4ccccc4n(-c4ccccc4)c21)n3-c1nc(-c2ccccc2)nc(-c2ccc(-c3cccc4c3sc3ccccc34)cc2)n1]

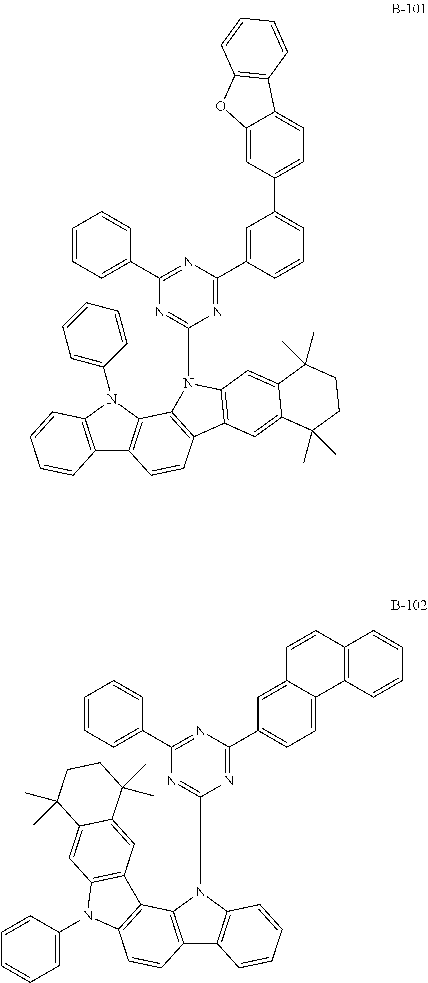 [SMILES: CC1(C)CCC(C)(C)c2cc3c(cc21)c1ccc2c4ccccc4n(-c4ccccc4)c2c1n3-c1nc(-c2ccccc2)nc(-c2cccc(-c3ccc4c(c3)oc3ccccc34)c2)n1 ; CC1(C)CCC(C)(C)c2cc3c(cc21)c1c(ccc2c4ccccc4n(-c4nc(-c5ccccc5)nc(-c5ccc6c(ccc7ccccc76)c5)n4)c21)n3-c1ccccc1]

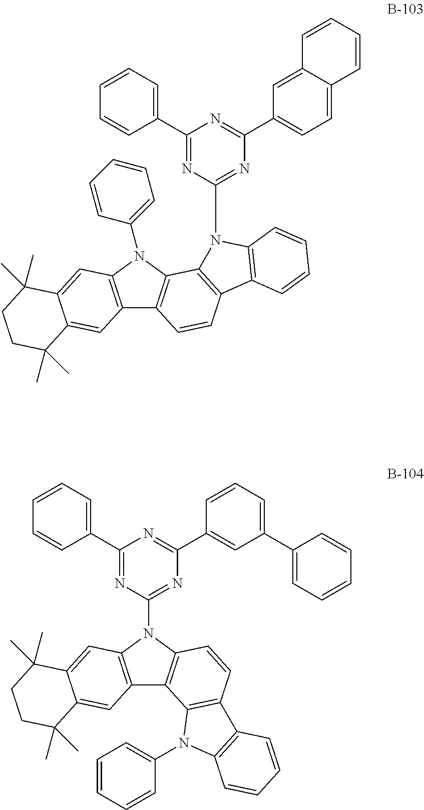 [SMILES: CC1(C)CCC(C)(C)c2cc3c(cc21)c1ccc2c4ccccc4n(-c4nc(-c5ccccc5)nc(-c5ccc6ccccc6c5)n4)c2c1n3-c1ccccc1 ; CC1(C)CCC(C)(C)c2cc3c(cc21)c1c(ccc2c4ccccc4n(-c4ccccc4)c21)n3-c1nc(-c2ccccc2)nc(-c2cccc(-c3ccccc3)c2)n1]

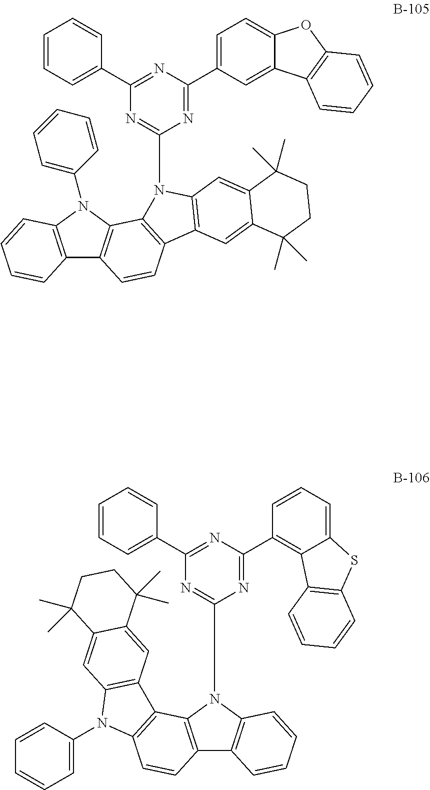 [SMILES: CC1(C)CCC(C)(C)c2cc3c(cc21)c1ccc2c4ccccc4n(-c4ccccc4)c2c1n3-c1nc(-c2ccccc2)nc(-c2ccc3oc4ccccc4c3c2)n1 ; CC1(C)CCC(C)(C)c2cc3c(cc21)c1c(ccc2c4ccccc4n(-c4nc(-c5ccccc5)nc(-c5cccc6sc7ccccc7c56)n4)c21)n3-c1ccccc1]

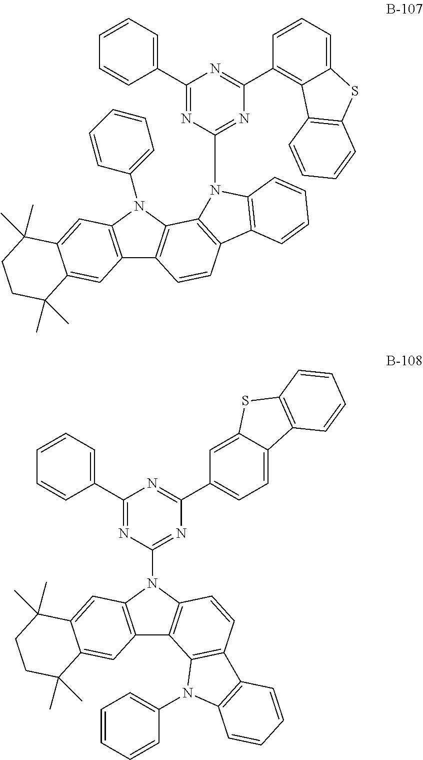 [SMILES: CC1(C)CCC(C)(C)c2cc3c(cc21)c1ccc2c4ccccc4n(-c4nc(-c5ccccc5)nc(-c5cccc6sc7ccccc7c56)n4)c2c1n3-c1ccccc1 ; CC1(C)CCC(C)(C)c2cc3c(cc21)c1c(ccc2c4ccccc4n(-c4ccccc4)c21)n3-c1nc(-c2ccccc2)nc(-c2ccc3c(c2)sc2ccccc23)n1]

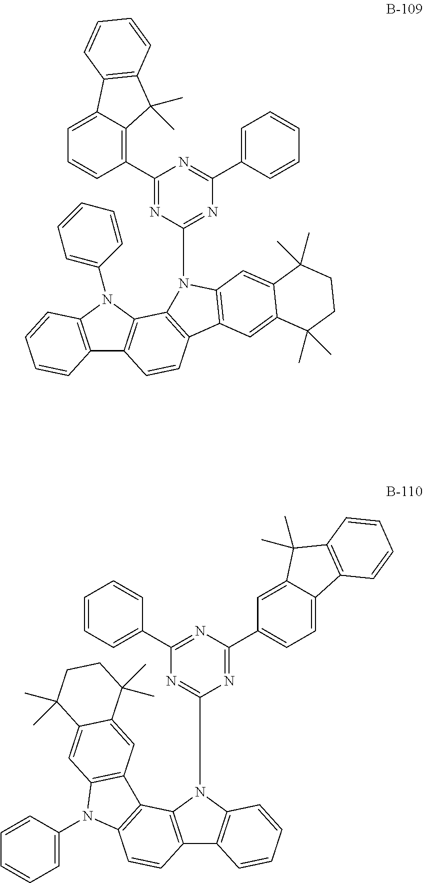 [SMILES: CC1(C)CCC(C)(C)c2cc3c(cc21)c1ccc2c4ccccc4n(-c4ccccc4)c2c1n3-c1nc(-c2ccccc2)nc(-c2cccc3c2C(C)(C)c2ccccc2-3)n1 ; CC1(C)CCC(C)(C)c2cc3c(cc21)c1c(ccc2c4ccccc4n(-c4nc(-c5ccccc5)nc(-c5ccc6c(c5)C(C)(C)c5ccccc5-6)n4)c21)n3-c1ccccc1]

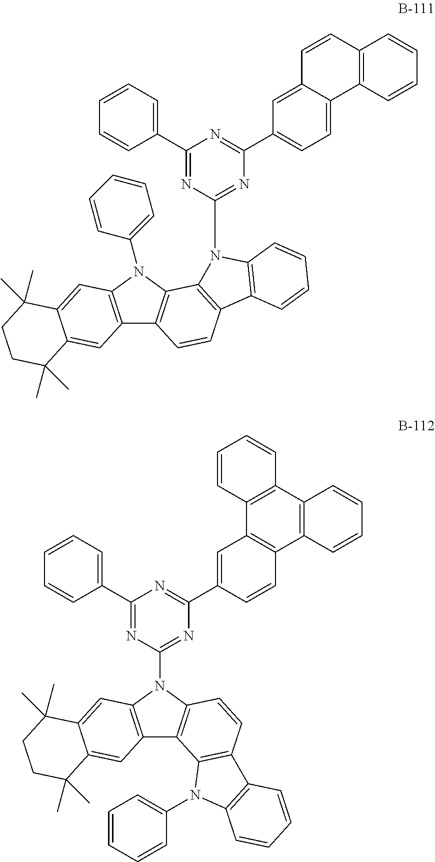 [SMILES: CC1(C)CCC(C)(C)c2cc3c(cc21)c1ccc2c4ccccc4n(-c4nc(-c5ccccc5)nc(-c5ccc6c(ccc7ccccc76)c5)n4)c2c1n3-c1ccccc1 ; CC1(C)CCC(C)(C)c2cc3c(cc21)c1c(ccc2c4ccccc4n(-c4ccccc4)c21)n3-c1nc(-c2ccccc2)nc(-c2ccc3c4ccccc4c4ccccc4c3c2)n1]

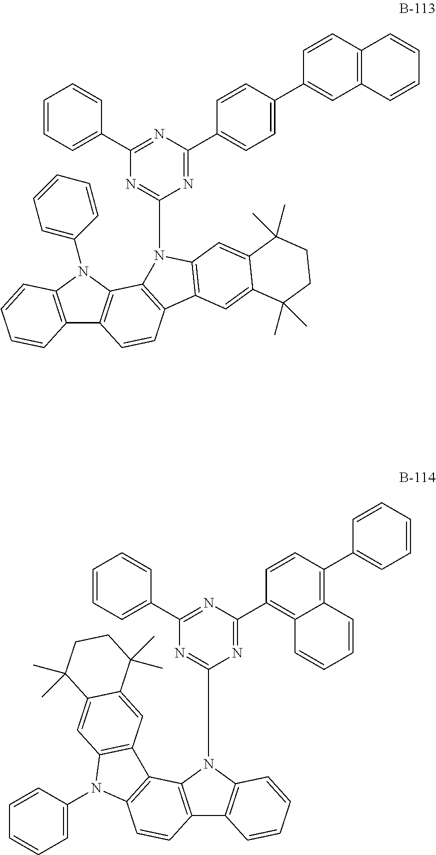 [SMILES: CC1(C)CCC(C)(C)c2cc3c(cc21)c1ccc2c4ccccc4n(-c4ccccc4)c2c1n3-c1nc(-c2ccccc2)nc(-c2ccc(-c3ccc4ccccc4c3)cc2)n1 ; CC1(C)CCC(C)(C)c2cc3c(cc21)c1c(ccc2c4ccccc4n(-c4nc(-c5ccccc5)nc(-c5ccc(-c6ccccc6)c6ccccc56)n4)c21)n3-c1ccccc1]

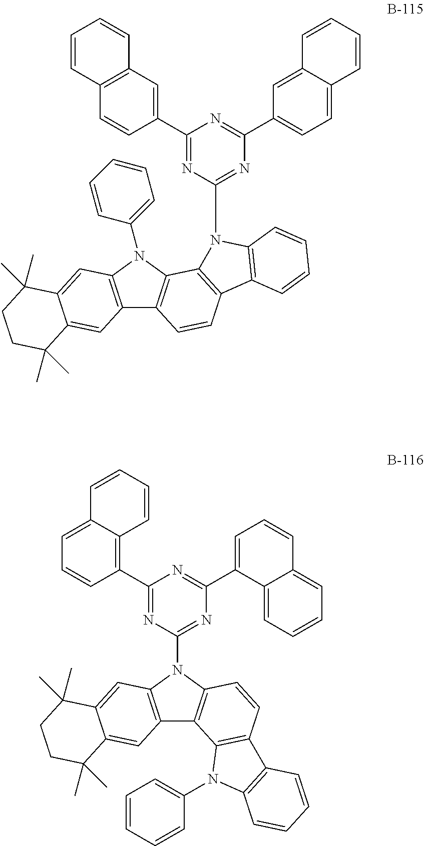 [SMILES: CC1(C)CCC(C)(C)c2cc3c(cc21)c1ccc2c4ccccc4n(-c4nc(-c5ccc6ccccc6c5)nc(-c5ccc6ccccc6c5)n4)c2c1n3-c1ccccc1 ; CC1(C)CCC(C)(C)c2cc3c(cc21)c1c(ccc2c4ccccc4n(-c4ccccc4)c21)n3-c1nc(-c2cccc3ccccc23)nc(-c2cccc3ccccc23)n1]

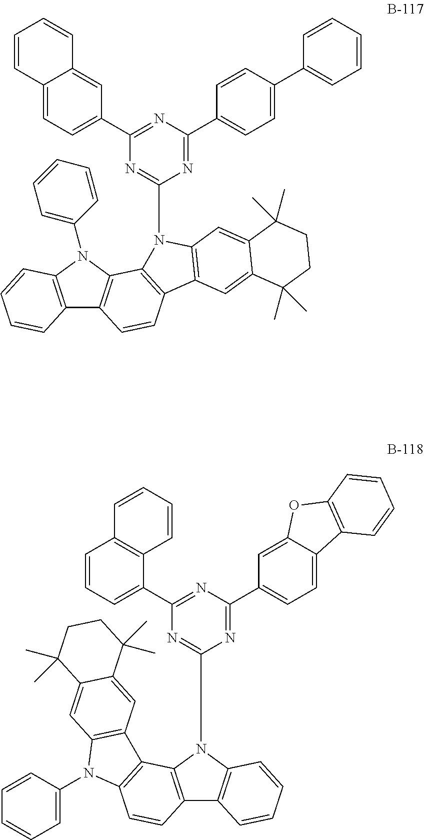 [SMILES: CC1(C)CCC(C)(C)c2cc3c(cc21)c1ccc2c4ccccc4n(-c4ccccc4)c2c1n3-c1nc(-c2ccc(-c3ccccc3)cc2)nc(-c2ccc3ccccc3c2)n1 ; CC1(C)CCC(C)(C)c2cc3c(cc21)c1c(ccc2c4ccccc4n(-c4nc(-c5ccc6c(c5)oc5ccccc56)nc(-c5cccc6ccccc56)n4)c21)n3-c1ccccc1]

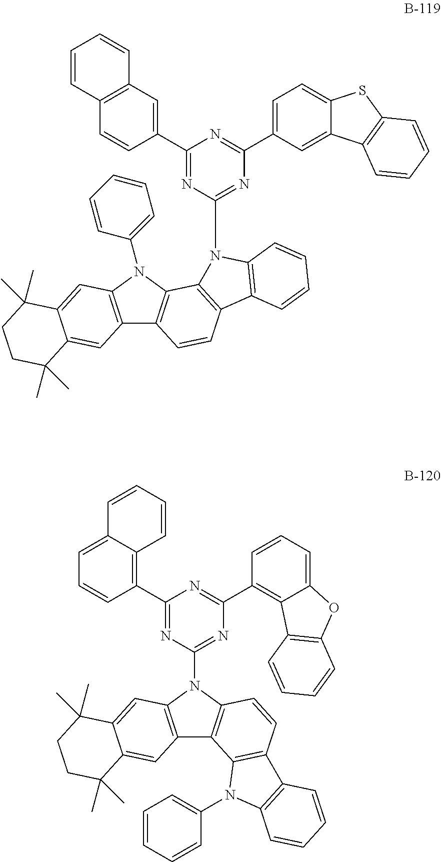 [SMILES: CC1(C)CCC(C)(C)c2cc3c(cc21)c1ccc2c4ccccc4n(-c4nc(-c5ccc6ccccc6c5)nc(-c5ccc6sc7ccccc7c6c5)n4)c2c1n3-c1ccccc1 ; CC1(C)CCC(C)(C)c2cc3c(cc21)c1c(ccc2c4ccccc4n(-c4ccccc4)c21)n3-c1nc(-c2cccc3ccccc23)nc(-c2cccc3oc4ccccc4c23)n1]

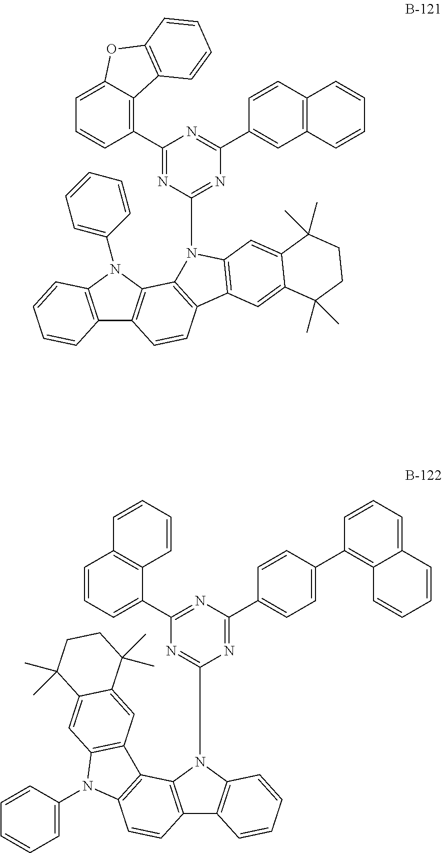 [SMILES: CC1(C)CCC(C)(C)c2cc3c(cc21)c1ccc2c4ccccc4n(-c4ccccc4)c2c1n3-c1nc(-c2ccc3ccccc3c2)nc(-c2cccc3oc4ccccc4c23)n1 ; CC1(C)CCC(C)(C)c2cc3c(cc21)c1c(ccc2c4ccccc4n(-c4nc(-c5ccc(-c6cccc7ccccc67)cc5)nc(-c5cccc6ccccc56)n4)c21)n3-c1ccccc1]

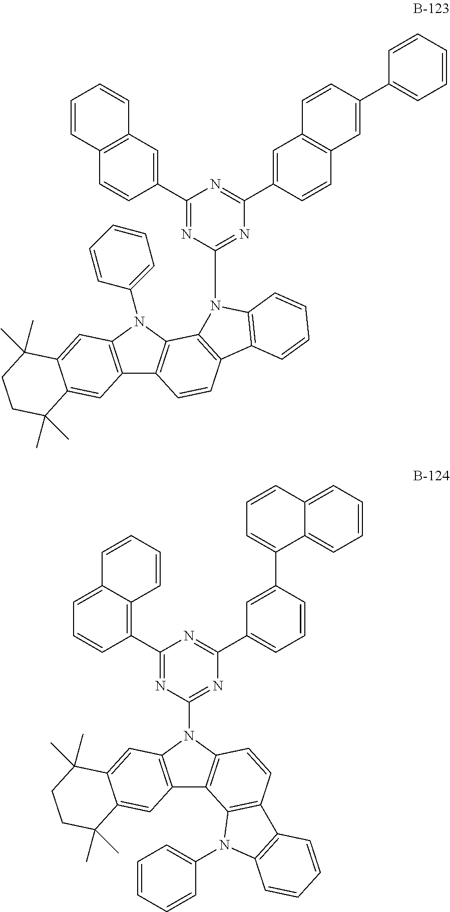 [SMILES: CC1(C)CCC(C)(C)c2cc3c(cc21)c1ccc2c4ccccc4n(-c4nc(-c5ccc6ccccc6c5)nc(-c5ccc6cc(-c7ccccc7)ccc6c5)n4)c2c1n3-c1ccccc1 ; CC1(C)CCC(C)(C)c2cc3c(cc21)c1c(ccc2c4ccccc4n(-c4ccccc4)c21)n3-c1nc(-c2cccc(-c3cccc4ccccc34)c2)nc(-c2cccc3ccccc23)n1]

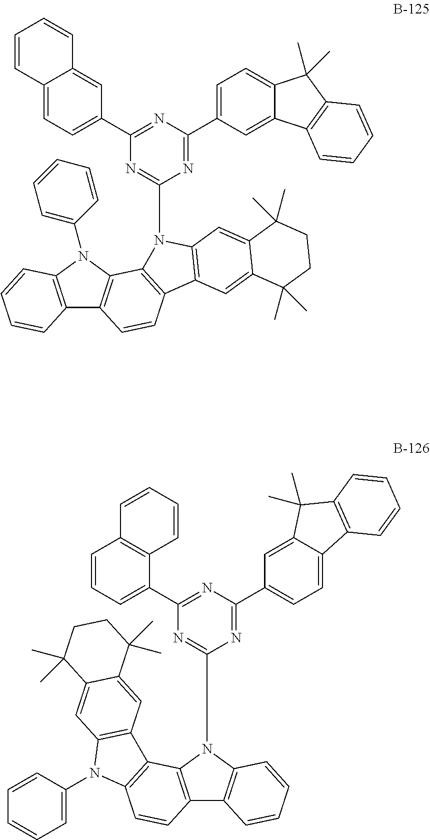 [SMILES: CC1(C)CCC(C)(C)c2cc3c(cc21)c1ccc2c4ccccc4n(-c4ccccc4)c2c1n3-c1nc(-c2ccc3c(c2)-c2ccccc2C3(C)C)nc(-c2ccc3ccccc3c2)n1 ; CC1(C)CCC(C)(C)c2cc3c(cc21)c1c(ccc2c4ccccc4n(-c4nc(-c5ccc6c(c5)C(C)(C)c5ccccc5-6)nc(-c5cccc6ccccc56)n4)c21)n3-c1ccccc1]

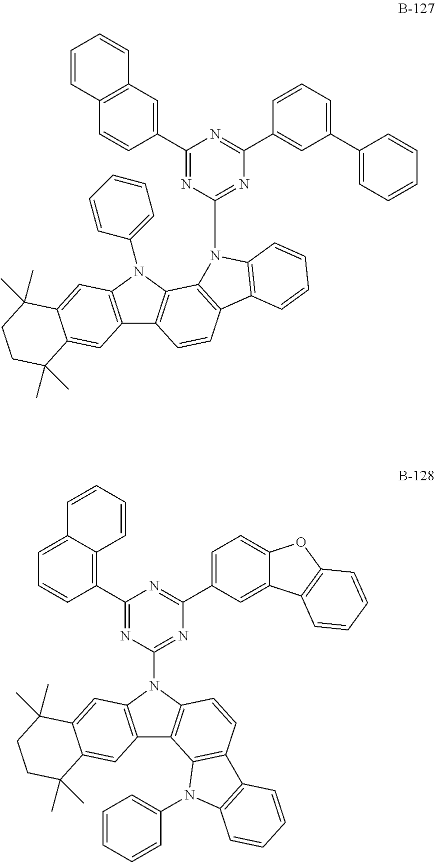 [SMILES: CC1(C)CCC(C)(C)c2cc3c(cc21)c1ccc2c4ccccc4n(-c4nc(-c5cccc(-c6ccccc6)c5)nc(-c5ccc6ccccc6c5)n4)c2c1n3-c1ccccc1 ; CC1(C)CCC(C)(C)c2cc3c(cc21)c1c(ccc2c4ccccc4n(-c4ccccc4)c21)n3-c1nc(-c2ccc3oc4ccccc4c3c2)nc(-c2cccc3ccccc23)n1]

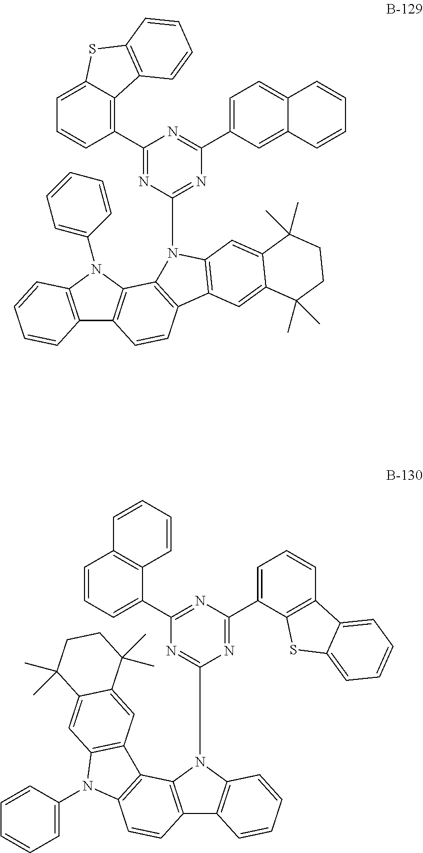 [SMILES: CC1(C)CCC(C)(C)c2cc3c(cc21)c1ccc2c4ccccc4n(-c4ccccc4)c2c1n3-c1nc(-c2ccc3ccccc3c2)nc(-c2cccc3sc4ccccc4c23)n1 ; CC1(C)CCC(C)(C)c2cc3c(cc21)c1c(ccc2c4ccccc4n(-c4nc(-c5cccc6ccccc56)nc(-c5cccc6c5sc5ccccc56)n4)c21)n3-c1ccccc1]

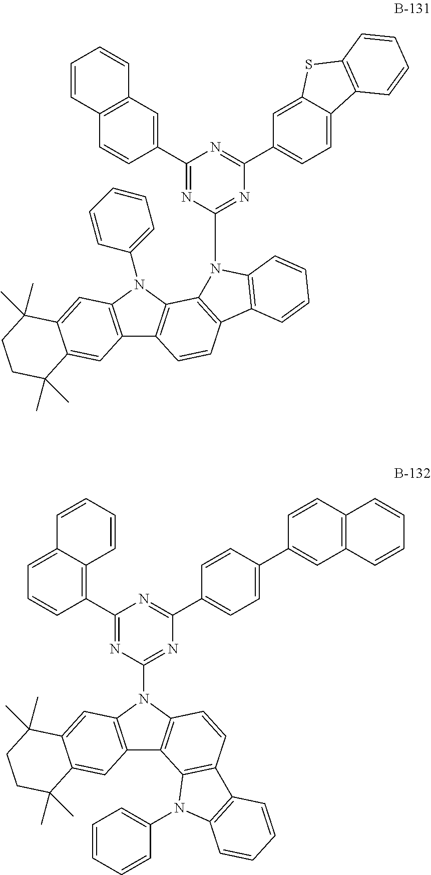 [SMILES: CC1(C)CCC(C)(C)c2cc3c(cc21)c1ccc2c4ccccc4n(-c4nc(-c5ccc6ccccc6c5)nc(-c5ccc6c(c5)sc5ccccc56)n4)c2c1n3-c1ccccc1 ; CC1(C)CCC(C)(C)c2cc3c(cc21)c1c(ccc2c4ccccc4n(-c4ccccc4)c21)n3-c1nc(-c2ccc(-c3ccc4ccccc4c3)cc2)nc(-c2cccc3ccccc23)n1]

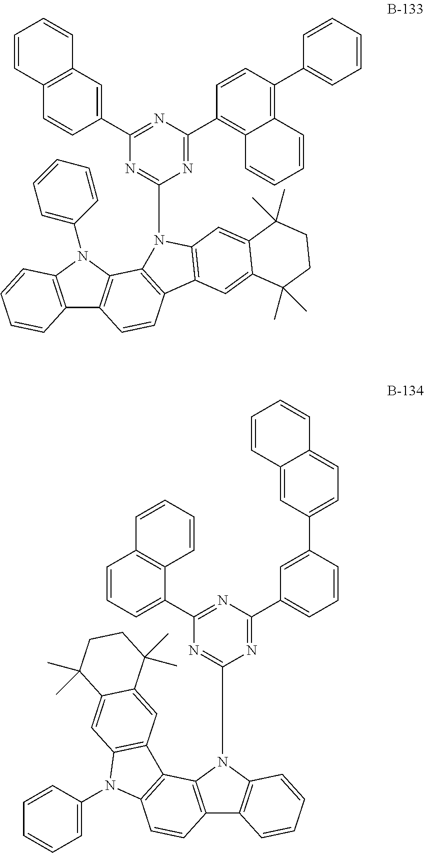 [SMILES: CC1(C)CCC(C)(C)c2cc3c(cc21)c1ccc2c4ccccc4n(-c4ccccc4)c2c1n3-c1nc(-c2ccc3ccccc3c2)nc(-c2ccc(-c3ccccc3)c3ccccc23)n1 ; CC1(C)CCC(C)(C)c2cc3c(cc21)c1c(ccc2c4ccccc4n(-c4nc(-c5cccc(-c6ccc7ccccc7c6)c5)nc(-c5cccc6ccccc56)n4)c21)n3-c1ccccc1]

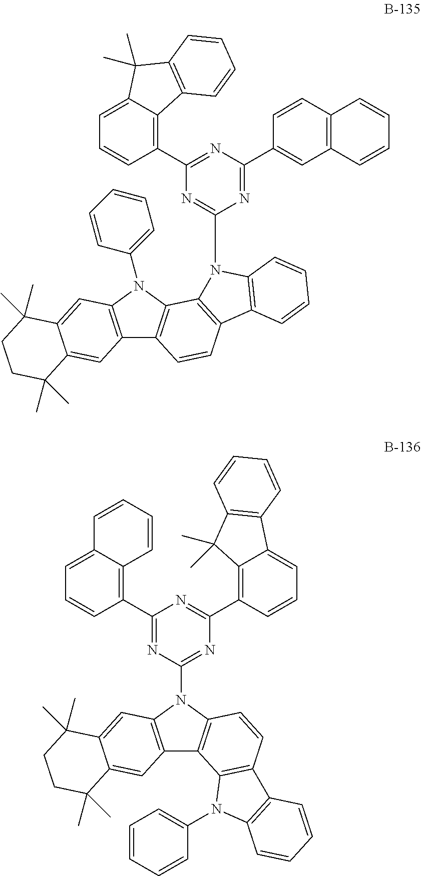 [SMILES: CC1(C)CCC(C)(C)c2cc3c(cc21)c1ccc2c4ccccc4n(-c4nc(-c5ccc6ccccc6c5)nc(-c5cccc6c5-c5ccccc5C6(C)C)n4)c2c1n3-c1ccccc1 ; CC1(C)CCC(C)(C)c2cc3c(cc21)c1c(ccc2c4ccccc4n(-c4ccccc4)c21)n3-c1nc(-c2cccc3c2C(C)(C)c2ccccc2-3)nc(-c2cccc3ccccc23)n1]

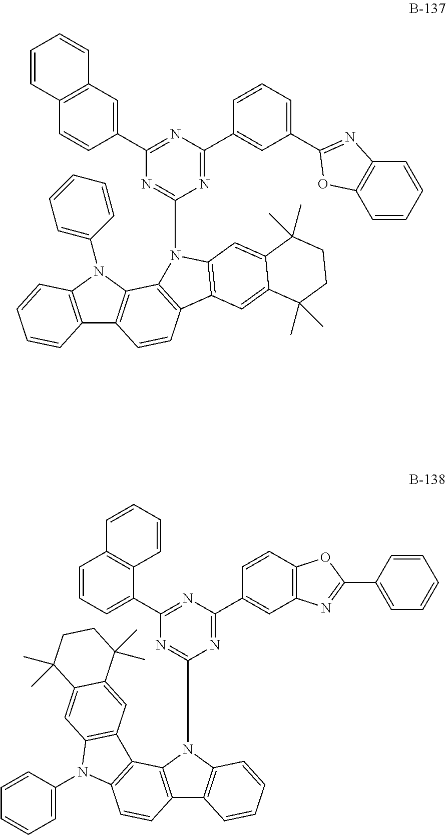 [SMILES: CC1(C)CCC(C)(C)c2cc3c(cc21)c1ccc2c4ccccc4n(-c4ccccc4)c2c1n3-c1nc(-c2cccc(-c3nc4ccccc4o3)c2)nc(-c2ccc3ccccc3c2)n1 ; CC1(C)CCC(C)(C)c2cc3c(cc21)c1c(ccc2c4ccccc4n(-c4nc(-c5ccc6oc(-c7ccccc7)nc6c5)nc(-c5cccc6ccccc56)n4)c21)n3-c1ccccc1]

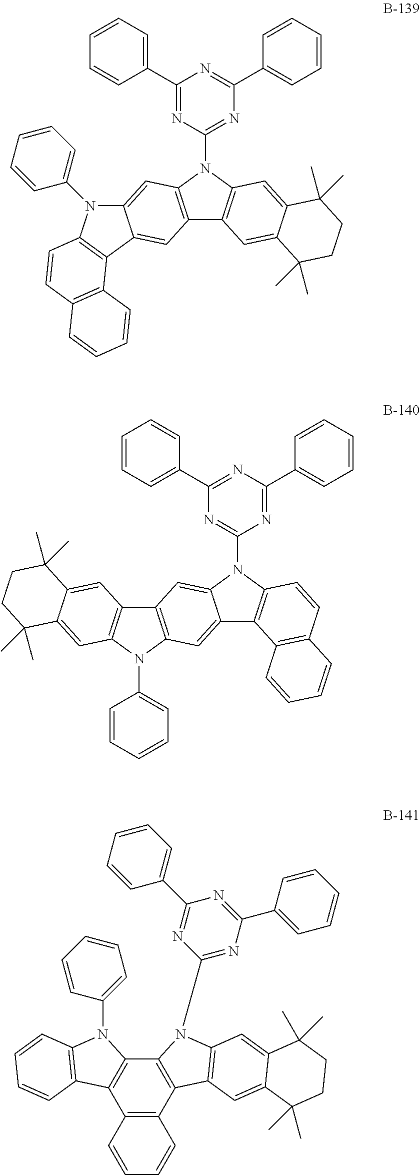

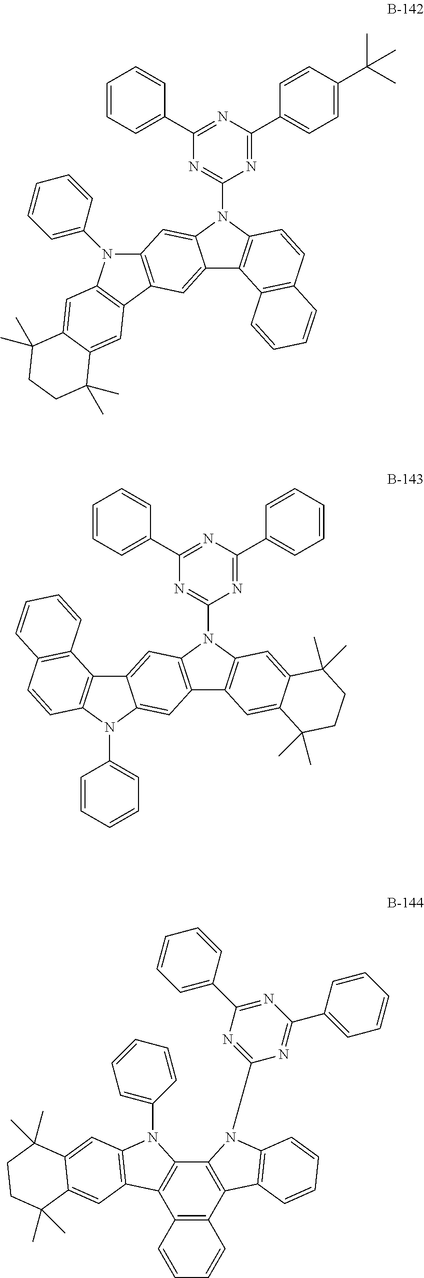 [SMILES: CC(C)(C)c1ccc(-c2nc(-c3ccccc3)nc(-n3c4cc5c(cc4c4c6ccccc6ccc43)c3cc4c(cc3n5-c3ccccc3)C(C)(C)CCC4(C)C)n2)cc1 ; CC1(C)CCC(C)(C)c2cc3c(cc21)c1cc2c(cc1n3-c1nc(-c3ccccc3)nc(-c3ccccc3)n1)c1c3ccccc3ccc1n2-c1ccccc1 ; CC1(C)CCC(C)(C)c2cc3c(cc21)c1c2ccccc2c2c4ccccc4n(-c4nc(-c5ccccc5)nc(-c5ccccc5)n4)c2c1n3-c1ccccc1]

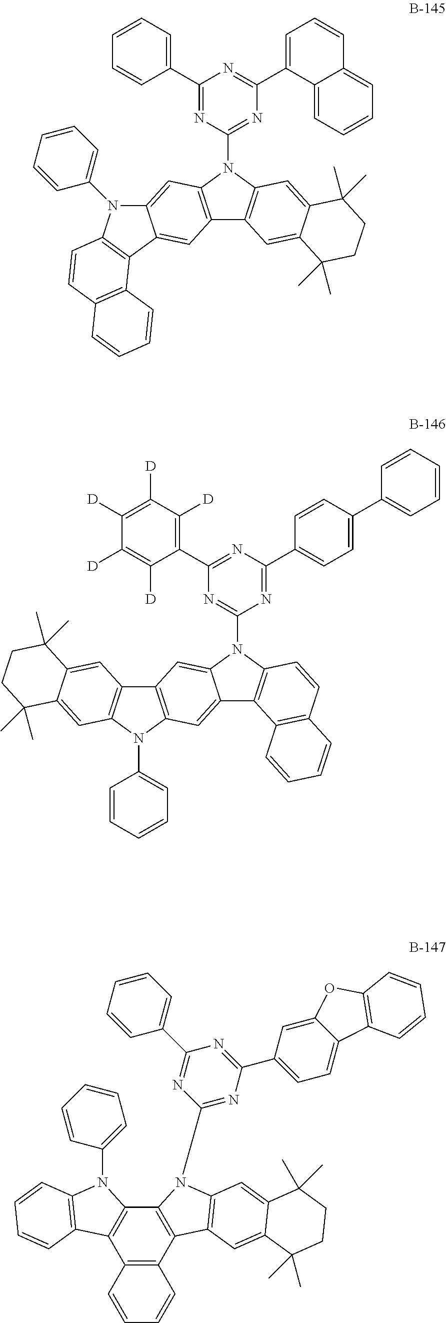 [SMILES: CC1(C)CCC(C)(C)c2cc3c(cc21)c1cc2c4c5ccccc5ccc4n(-c4ccccc4)c2cc1n3-c1nc(-c2ccccc2)nc(-c2cccc3ccccc23)n1 ; [2H]c1c([2H])c([2H])c(-c2nc(-c3ccc(-c4ccccc4)cc3)nc(-n3c4cc5c6cc7c(cc6n(-c6ccccc6)c5cc4c4c5ccccc5ccc43)C(C)(C)CCC7(C)C)n2)c([2H])c1[2H] ; CC1(C)CCC(C)(C)c2cc3c(cc21)c1c2ccccc2c2c4ccccc4n(-c4ccccc4)c2c1n3-c1nc(-c2ccccc2)nc(-c2ccc3c(c2)oc2ccccc23)n1]

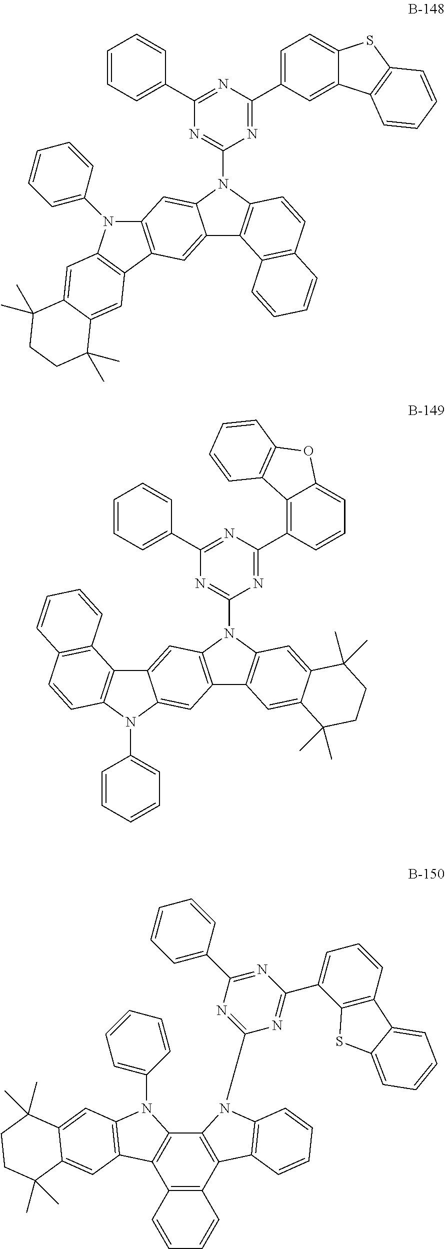 [SMILES: CC1(C)CCC(C)(C)c2cc3c(cc21)c1cc2c4c5ccccc5ccc4n(-c4nc(-c5ccccc5)nc(-c5ccc6sc7ccccc7c6c5)n4)c2cc1n3-c1ccccc1 ; CC1(C)CCC(C)(C)c2cc3c(cc21)c1cc2c(cc1n3-c1nc(-c3ccccc3)nc(-c3cccc4oc5ccccc5c34)n1)c1c3ccccc3ccc1n2-c1ccccc1 ; CC1(C)CCC(C)(C)c2cc3c(cc21)c1c2ccccc2c2c4ccccc4n(-c4nc(-c5ccccc5)nc(-c5cccc6c5sc5ccccc56)n4)c2c1n3-c1ccccc1]

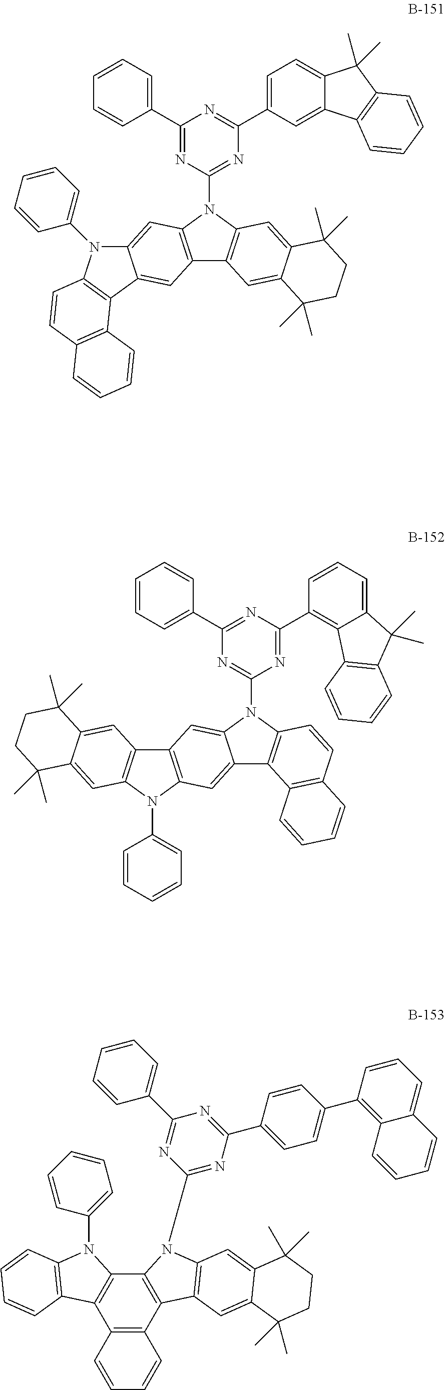

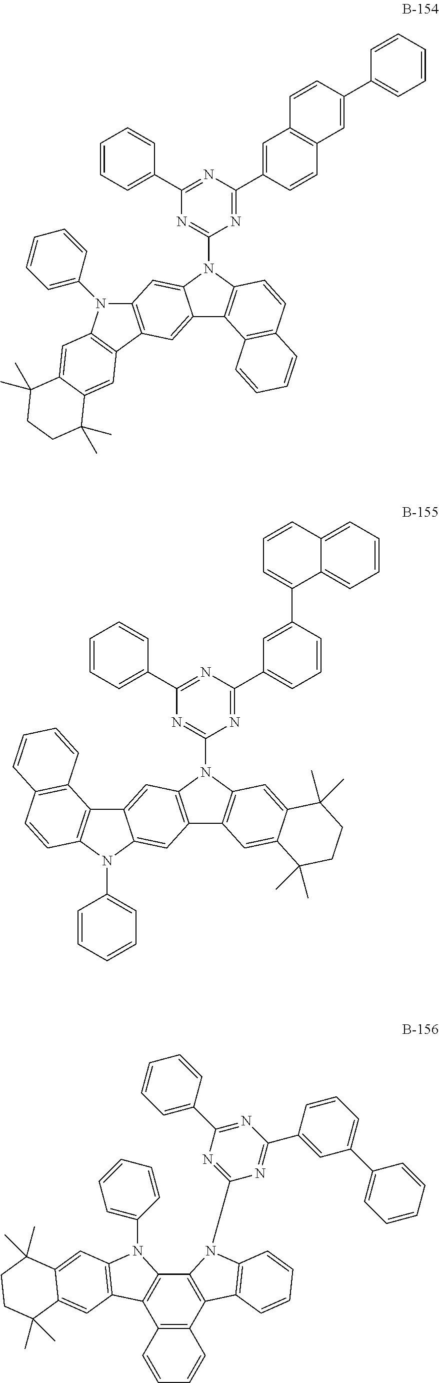 [SMILES: CC1(C)CCC(C)(C)c2cc3c(cc21)c1cc2c4c5ccccc5ccc4n(-c4nc(-c5ccccc5)nc(-c5ccc6cc(-c7ccccc7)ccc6c5)n4)c2cc1n3-c1ccccc1 ; CC1(C)CCC(C)(C)c2cc3c(cc21)c1cc2c(cc1n3-c1nc(-c3ccccc3)nc(-c3cccc(-c4cccc5ccccc45)c3)n1)c1c3ccccc3ccc1n2-c1ccccc1 ; CC1(C)CCC(C)(C)c2cc3c(cc21)c1c2ccccc2c2c4ccccc4n(-c4nc(-c5ccccc5)nc(-c5cccc(-c6ccccc6)c5)n4)c2c1n3-c1ccccc1]

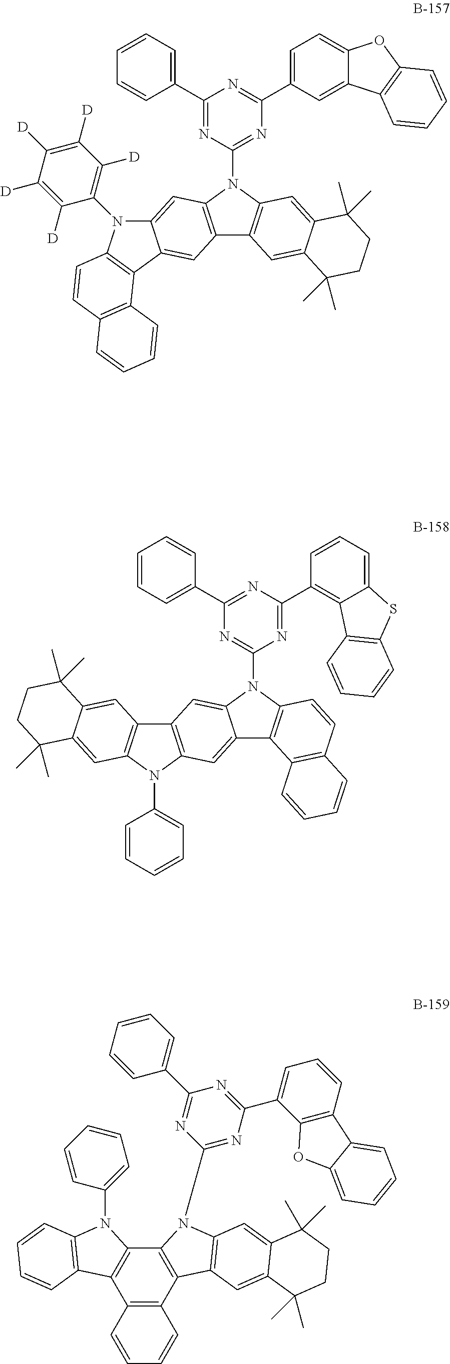 [SMILES: [2H]c1c([2H])c([2H])c(-n2c3cc4c(cc3c3c5ccccc5ccc32)c2cc3c(cc2n4-c2nc(-c4ccccc4)nc(-c4ccc5oc6ccccc6c5c4)n2)C(C)(C)CCC3(C)C)c([2H])c1[2H] ; CC1(C)CCC(C)(C)c2cc3c(cc21)c1cc2c(cc1n3-c1ccccc1)c1c3ccccc3ccc1n2-c1nc(-c2ccccc2)nc(-c2cccc3sc4ccccc4c23)n1 ; CC1(C)CCC(C)(C)c2cc3c(cc21)c1c2ccccc2c2c4ccccc4n(-c4ccccc4)c2c1n3-c1nc(-c2ccccc2)nc(-c2cccc3c2oc2ccccc23)n1]

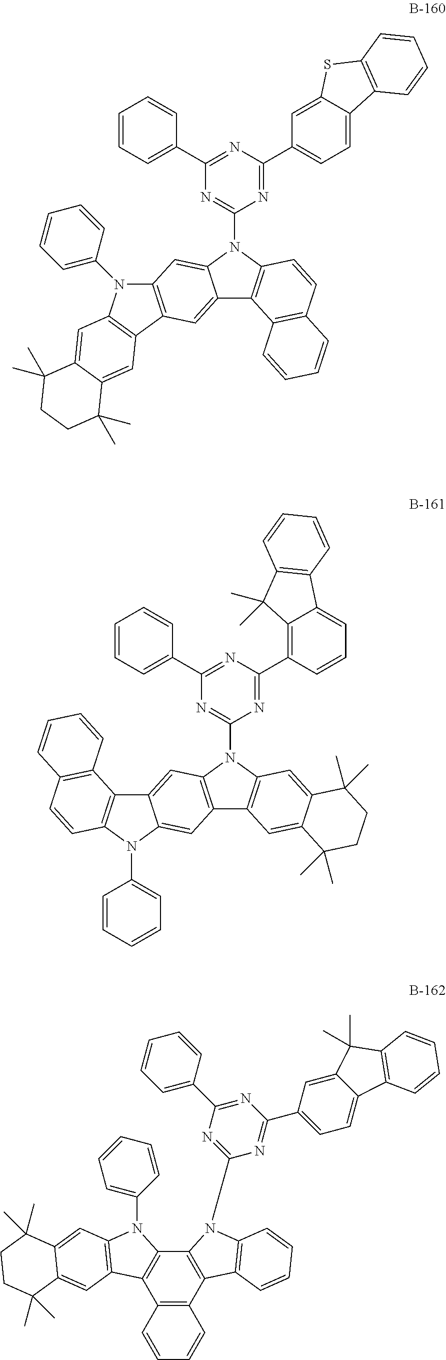 [SMILES: CC1(C)CCC(C)(C)c2cc3c(cc21)c1cc2c4c5ccccc5ccc4n(-c4nc(-c5ccccc5)nc(-c5ccc6c(c5)sc5ccccc56)n4)c2cc1n3-c1ccccc1 ; CC1(C)CCC(C)(C)c2cc3c(cc21)c1cc2c(cc1n3-c1nc(-c3ccccc3)nc(-c3cccc4c3C(C)(C)c3ccccc3-4)n1)c1c3ccccc3ccc1n2-c1ccccc1 ; CC1(C)CCC(C)(C)c2cc3c(cc21)c1c2ccccc2c2c4ccccc4n(-c4nc(-c5ccccc5)nc(-c5ccc6c(c5)C(C)(C)c5ccccc5-6)n4)c2c1n3-c1ccccc1]

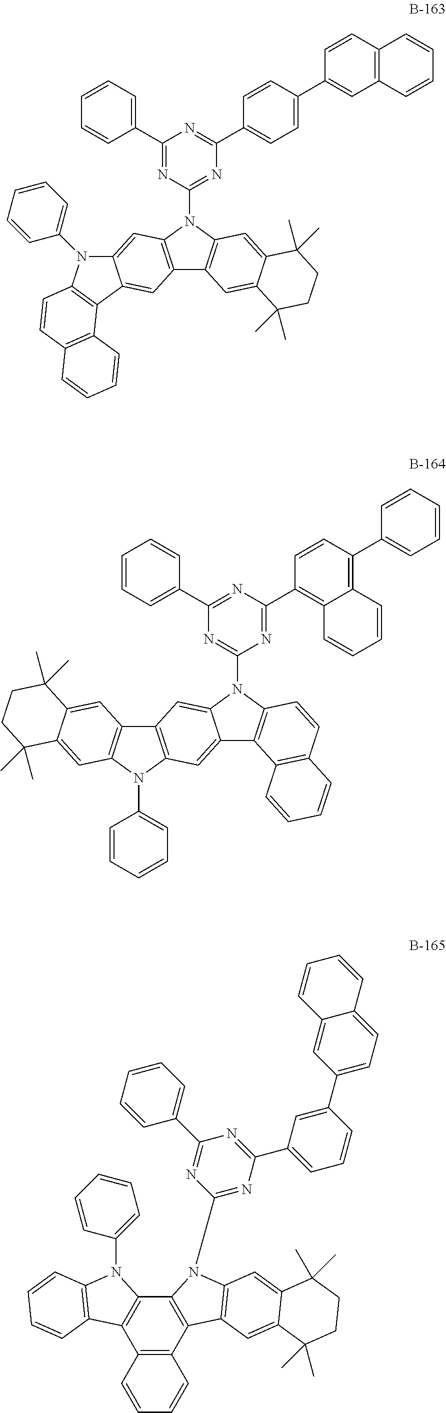 [SMILES: CC1(C)CCC(C)(C)c2cc3c(cc21)c1cc2c4c5ccccc5ccc4n(-c4ccccc4)c2cc1n3-c1nc(-c2ccccc2)nc(-c2ccc(-c3ccc4ccccc4c3)cc2)n1 ; CC1(C)CCC(C)(C)c2cc3c(cc21)c1cc2c(cc1n3-c1ccccc1)c1c3ccccc3ccc1n2-c1nc(-c2ccccc2)nc(-c2ccc(-c3ccccc3)c3ccccc23)n1 ; CC1(C)CCC(C)(C)c2cc3c(cc21)c1c2ccccc2c2c4ccccc4n(-c4ccccc4)c2c1n3-c1nc(-c2ccccc2)nc(-c2cccc(-c3ccc4ccccc4c3)c2)n1]

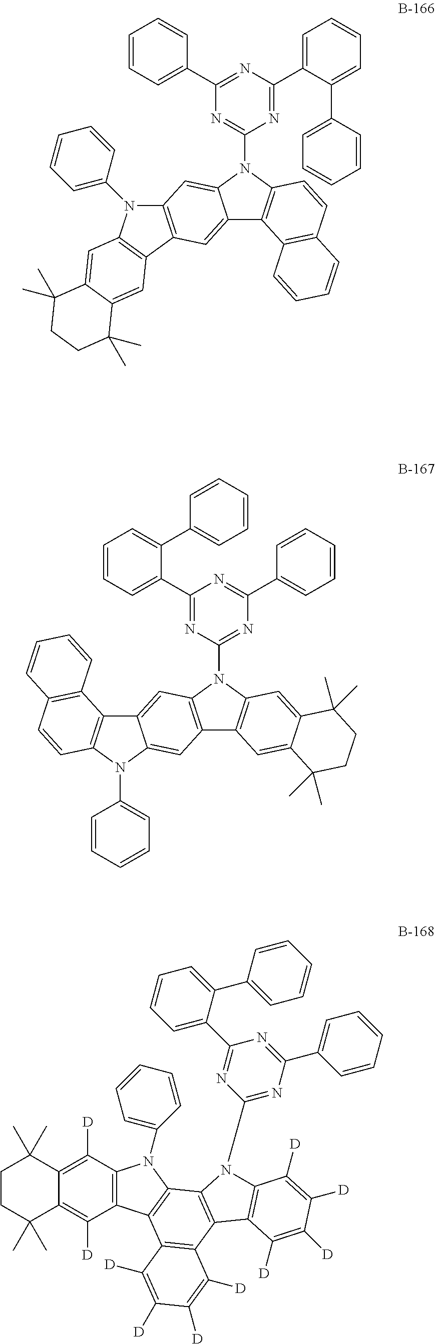 [SMILES: CC1(C)CCC(C)(C)c2cc3c(cc21)c1cc2c4c5ccccc5ccc4n(-c4nc(-c5ccccc5)nc(-c5ccccc5-c5ccccc5)n4)c2cc1n3-c1ccccc1 ; CC1(C)CCC(C)(C)c2cc3c(cc21)c1cc2c(cc1n3-c1nc(-c3ccccc3)nc(-c3ccccc3-c3ccccc3)n1)c1c3ccccc3ccc1n2-c1ccccc1 ; [2H]c1c([2H])c([2H])c2c(c1[2H])c1c3c([2H])c4c(c([2H])c3n(-c3ccccc3)c1c1c2c2c([2H])c([2H])c([2H])c([2H])c2n1-c1nc(-c2ccccc2)nc(-c2ccccc2-c2ccccc2)n1)C(C)(C)CCC4(C)C]

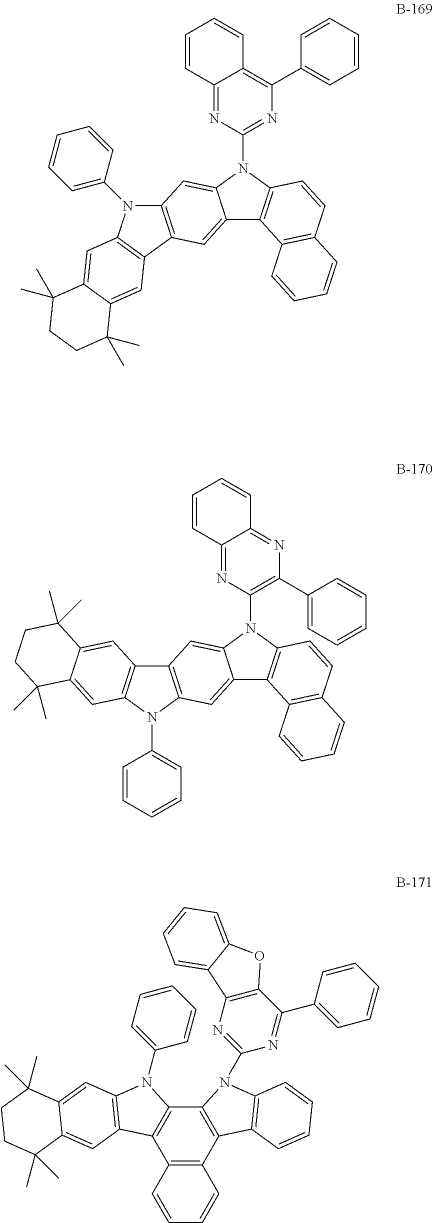 [SMILES: CC1(C)CCC(C)(C)c2cc3c(cc21)c1cc2c4c5ccccc5ccc4n(-c4nc(-c5ccccc5)c5ccccc5n4)c2cc1n3-c1ccccc1 ; CC1(C)CCC(C)(C)c2cc3c(cc21)c1cc2c(cc1n3-c1ccccc1)c1c3ccccc3ccc1n2-c1nc2ccccc2nc1-c1ccccc1 ; CC1(C)CCC(C)(C)c2cc3c(cc21)c1c2ccccc2c2c4ccccc4n(-c4nc(-c5ccccc5)c5oc6ccccc6c5n4)c2c1n3-c1ccccc1]

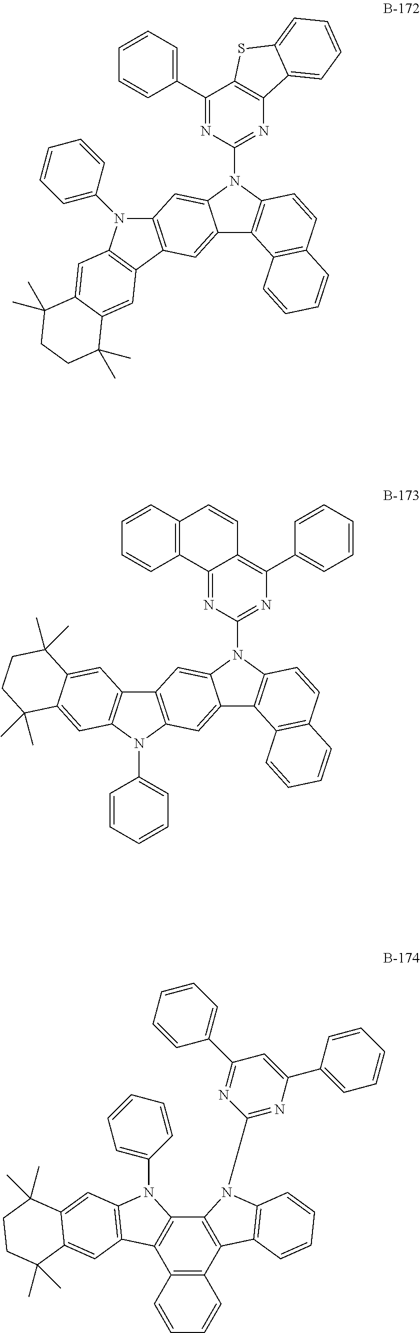 [SMILES: CC1(C)CCC(C)(C)c2cc3c(cc21)c1cc2c4c5ccccc5ccc4n(-c4nc(-c5ccccc5)c5sc6ccccc6c5n4)c2cc1n3-c1ccccc1 ; CC1(C)CCC(C)(C)c2cc3c(cc21)c1cc2c(cc1n3-c1ccccc1)c1c3ccccc3ccc1n2-c1nc(-c2ccccc2)c2ccc3ccccc3c2n1 ; CC1(C)CCC(C)(C)c2cc3c(cc21)c1c2ccccc2c2c4ccccc4n(-c4nc(-c5ccccc5)cc(-c5ccccc5)n4)c2c1n3-c1ccccc1]

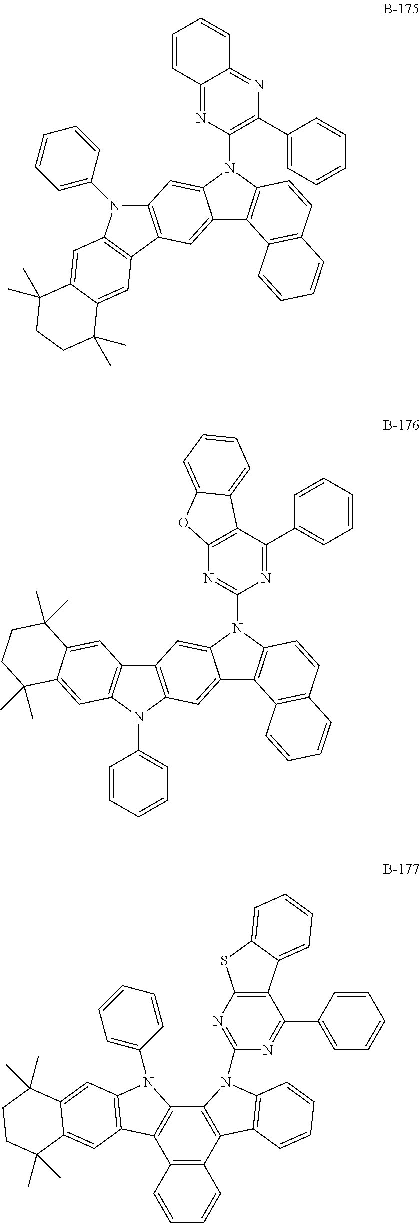 [SMILES: CC1(C)CCC(C)(C)c2cc3c(cc21)c1cc2c4c5ccccc5ccc4n(-c4nc5ccccc5nc4-c4ccccc4)c2cc1n3-c1ccccc1 ; CC1(C)CCC(C)(C)c2cc3c(cc21)c1cc2c(cc1n3-c1ccccc1)c1c3ccccc3ccc1n2-c1nc(-c2ccccc2)c2c(n1)oc1ccccc12 ; CC1(C)CCC(C)(C)c2cc3c(cc21)c1c2ccccc2c2c4ccccc4n(-c4nc(-c5ccccc5)c5c(n4)sc4ccccc45)c2c1n3-c1ccccc1]

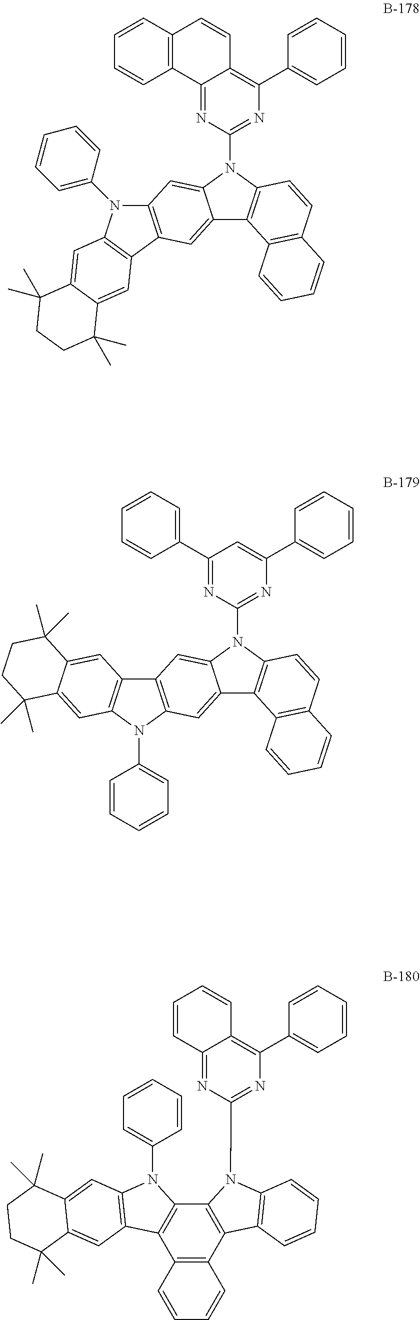 [SMILES: CC1(C)CCC(C)(C)c2cc3c(cc21)c1cc2c4c5ccccc5ccc4n(-c4nc(-c5ccccc5)c5ccc6ccccc6c5n4)c2cc1n3-c1ccccc1 ; CC1(C)CCC(C)(C)c2cc3c(cc21)c1cc2c(cc1n3-c1ccccc1)c1c3ccccc3ccc1n2-c1nc(-c2ccccc2)cc(-c2ccccc2)n1 ; CC1(C)CCC(C)(C)c2cc3c(cc21)c1c2ccccc2c2c4ccccc4n(-c4nc(-c5ccccc5)c5ccccc5n4)c2c1n3-c1ccccc1]

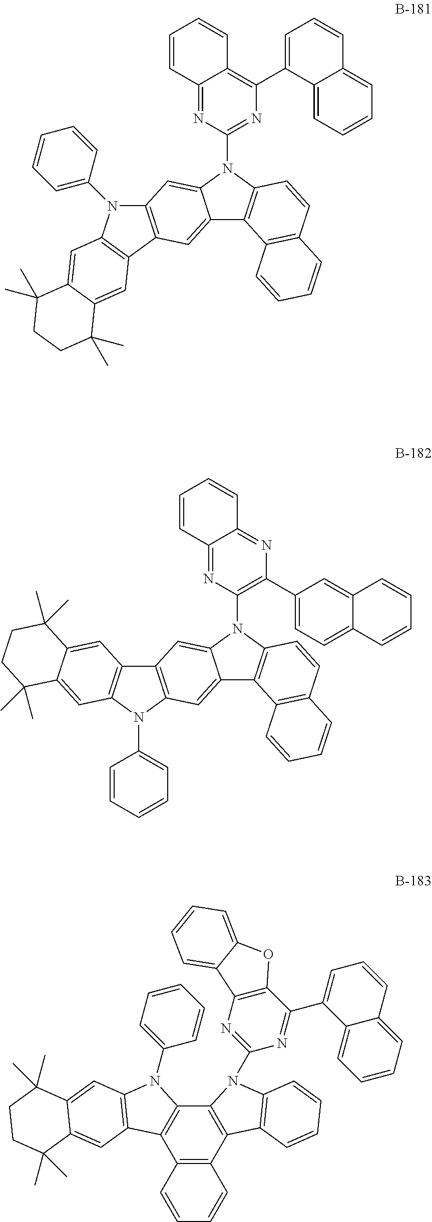 [SMILES: CC1(C)CCC(C)(C)c2cc3c(cc21)c1cc2c4c5ccccc5ccc4n(-c4nc(-c5cccc6ccccc56)c5ccccc5n4)c2cc1n3-c1ccccc1 ; CC1(C)CCC(C)(C)c2cc3c(cc21)c1cc2c(cc1n3-c1ccccc1)c1c3ccccc3ccc1n2-c1nc2ccccc2nc1-c1ccc2ccccc2c1 ; CC1(C)CCC(C)(C)c2cc3c(cc21)c1c2ccccc2c2c4ccccc4n(-c4nc(-c5cccc6ccccc56)c5oc6ccccc6c5n4)c2c1n3-c1ccccc1]

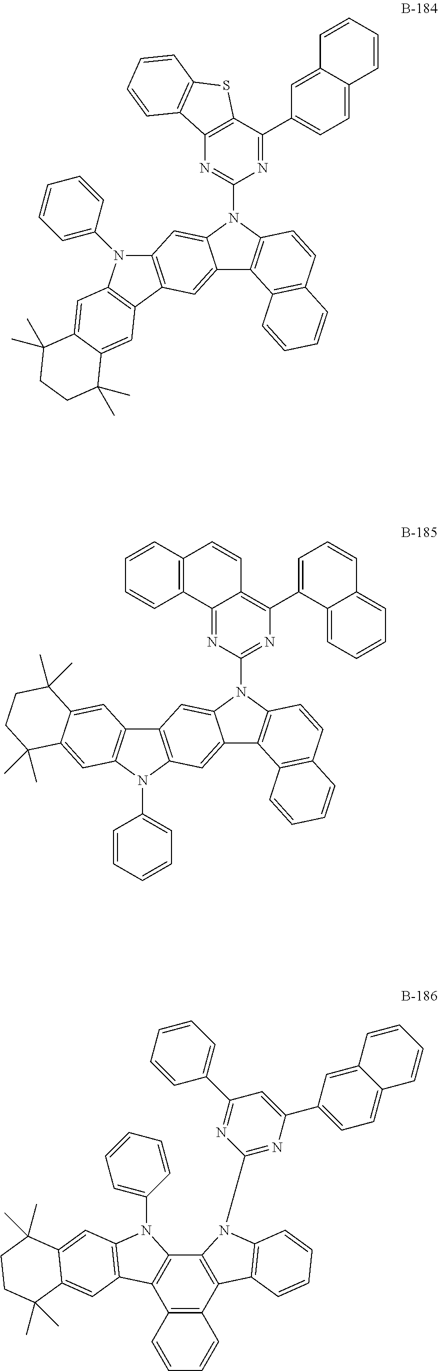 [SMILES: CC1(C)CCC(C)(C)c2cc3c(cc21)c1cc2c4c5ccccc5ccc4n(-c4nc(-c5ccc6ccccc6c5)c5sc6ccccc6c5n4)c2cc1n3-c1ccccc1 ; CC1(C)CCC(C)(C)c2cc3c(cc21)c1cc2c(cc1n3-c1ccccc1)c1c3ccccc3ccc1n2-c1nc(-c2cccc3ccccc23)c2ccc3ccccc3c2n1 ; CC1(C)CCC(C)(C)c2cc3c(cc21)c1c2ccccc2c2c4ccccc4n(-c4nc(-c5ccccc5)cc(-c5ccc6ccccc6c5)n4)c2c1n3-c1ccccc1]

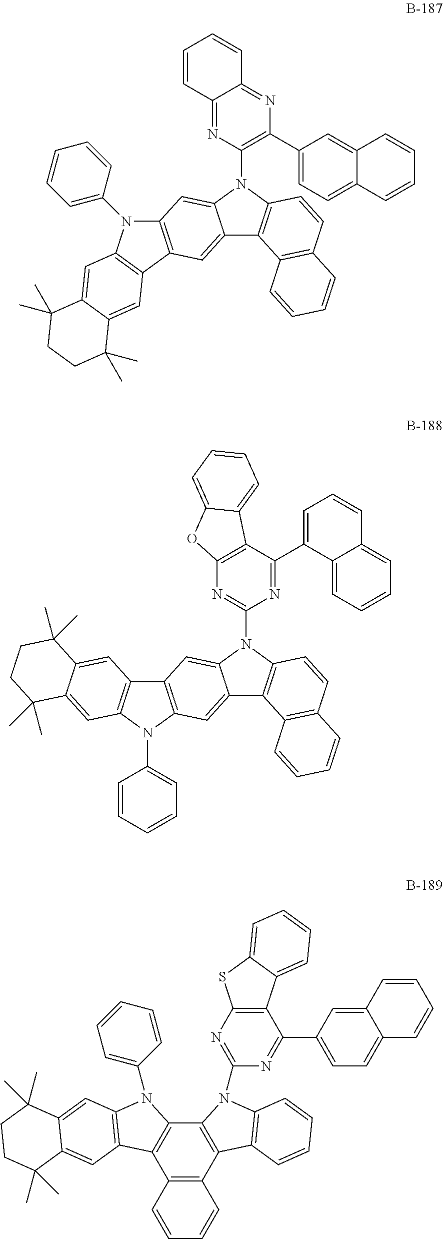 [SMILES: CC1(C)CCC(C)(C)c2cc3c(cc21)c1cc2c4c5ccccc5ccc4n(-c4nc5ccccc5nc4-c4ccc5ccccc5c4)c2cc1n3-c1ccccc1 ; CC1(C)CCC(C)(C)c2cc3c(cc21)c1cc2c(cc1n3-c1ccccc1)c1c3ccccc3ccc1n2-c1nc(-c2cccc3ccccc23)c2c(n1)oc1ccccc12 ; CC1(C)CCC(C)(C)c2cc3c(cc21)c1c2ccccc2c2c4ccccc4n(-c4nc(-c5ccc6ccccc6c5)c5c(n4)sc4ccccc45)c2c1n3-c1ccccc1]

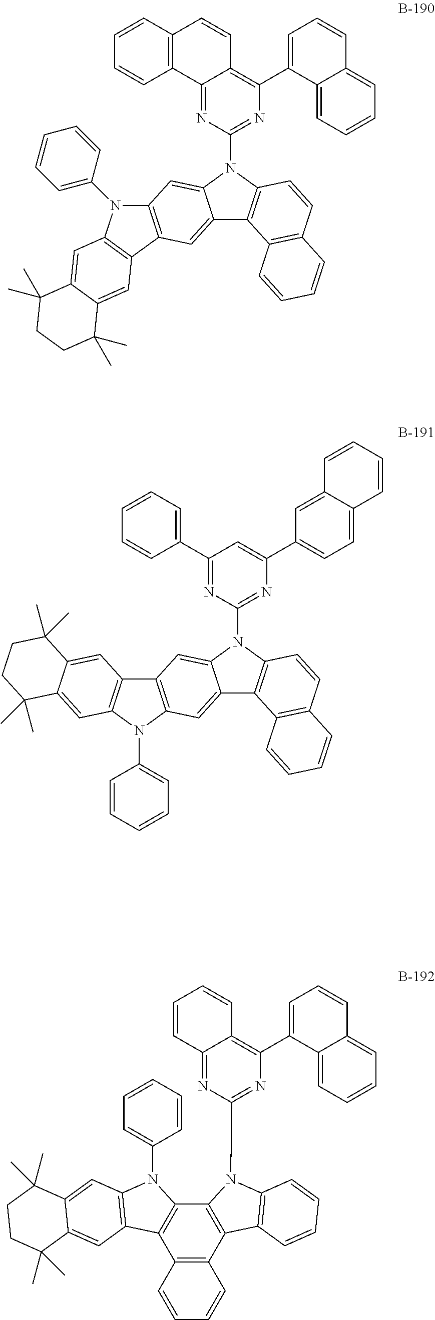 [SMILES: CC1(C)CCC(C)(C)c2cc3c(cc21)c1cc2c4c5ccccc5ccc4n(-c4nc(-c5cccc6ccccc56)c5ccc6ccccc6c5n4)c2cc1n3-c1ccccc1 ; CC1(C)CCC(C)(C)c2cc3c(cc21)c1cc2c(cc1n3-c1ccccc1)c1c3ccccc3ccc1n2-c1nc(-c2ccccc2)cc(-c2ccc3ccccc3c2)n1 ; CC1(C)CCC(C)(C)c2cc3c(cc21)c1c2ccccc2c2c4ccccc4n(-c4nc(-c5cccc6ccccc56)c5ccccc5n4)c2c1n3-c1ccccc1]

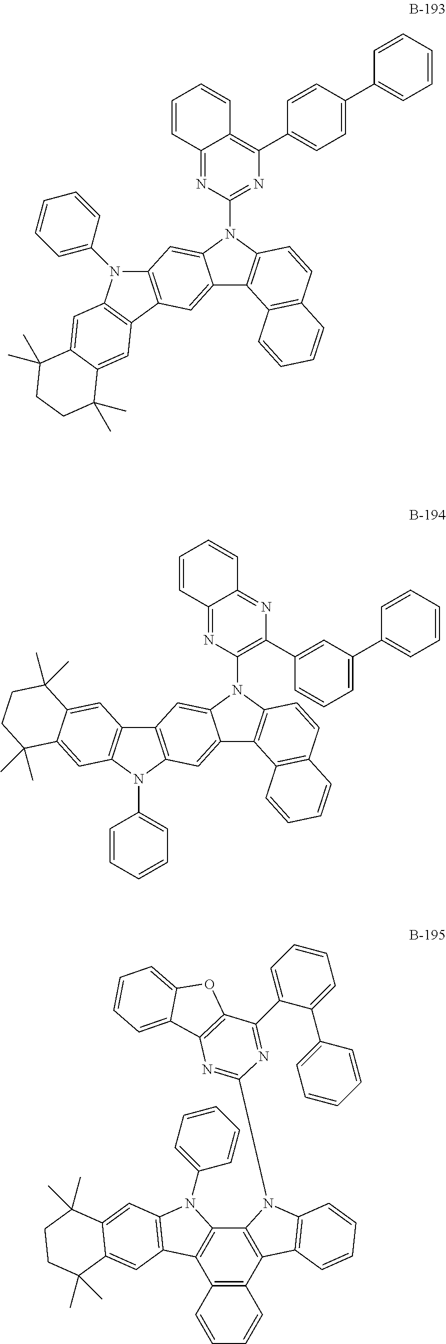 [SMILES: CC1(C)CCC(C)(C)c2cc3c(cc21)c1cc2c4c5ccccc5ccc4n(-c4nc(-c5ccc(-c6ccccc6)cc5)c5ccccc5n4)c2cc1n3-c1ccccc1 ; CC1(C)CCC(C)(C)c2cc3c(cc21)c1cc2c(cc1n3-c1ccccc1)c1c3ccccc3ccc1n2-c1nc2ccccc2nc1-c1cccc(-c2ccccc2)c1 ; CC1(C)CCC(C)(C)c2cc3c(cc21)c1c2ccccc2c2c4ccccc4n(-c4nc(-c5ccccc5-c5ccccc5)c5oc6ccccc6c5n4)c2c1n3-c1ccccc1]

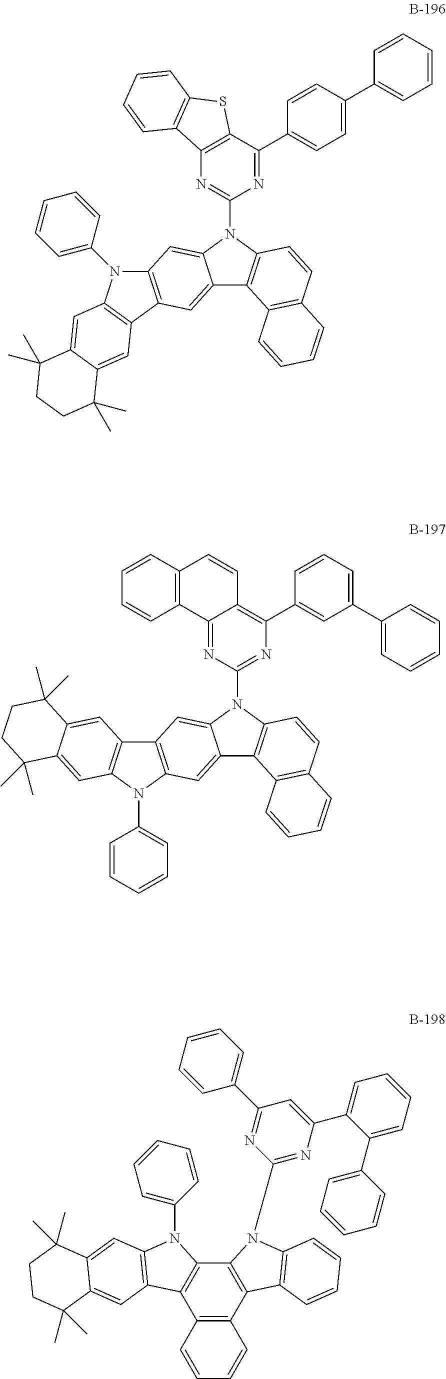 [SMILES: CC1(C)CCC(C)(C)c2cc3c(cc21)c1cc2c4c5ccccc5ccc4n(-c4nc(-c5ccc(-c6ccccc6)cc5)c5sc6ccccc6c5n4)c2cc1n3-c1ccccc1 ; CC1(C)CCC(C)(C)c2cc3c(cc21)c1cc2c(cc1n3-c1ccccc1)c1c3ccccc3ccc1n2-c1nc(-c2cccc(-c3ccccc3)c2)c2ccc3ccccc3c2n1 ; CC1(C)CCC(C)(C)c2cc3c(cc21)c1c2ccccc2c2c4ccccc4n(-c4nc(-c5ccccc5)cc(-c5ccccc5-c5ccccc5)n4)c2c1n3-c1ccccc1]

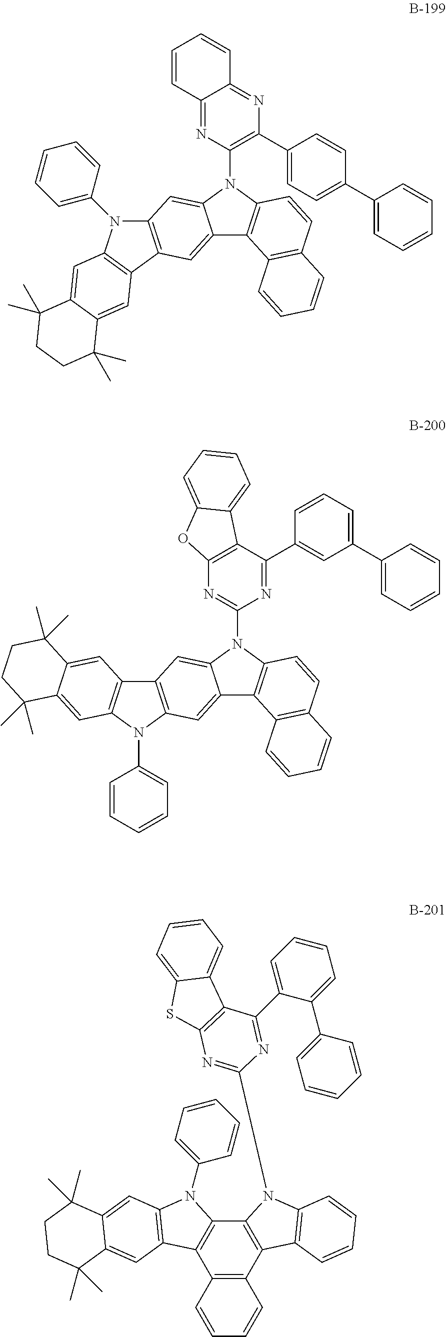 [SMILES: CC1(C)CCC(C)(C)c2cc3c(cc21)c1cc2c4c5ccccc5ccc4n(-c4nc5ccccc5nc4-c4ccc(-c5ccccc5)cc4)c2cc1n3-c1ccccc1 ; CC1(C)CCC(C)(C)c2cc3c(cc21)c1cc2c(cc1n3-c1ccccc1)c1c3ccccc3ccc1n2-c1nc(-c2cccc(-c3ccccc3)c2)c2c(n1)oc1ccccc12 ; CC1(C)CCC(C)(C)c2cc3c(cc21)c1c2ccccc2c2c4ccccc4n(-c4nc(-c5ccccc5-c5ccccc5)c5c(n4)sc4ccccc45)c2c1n3-c1ccccc1]

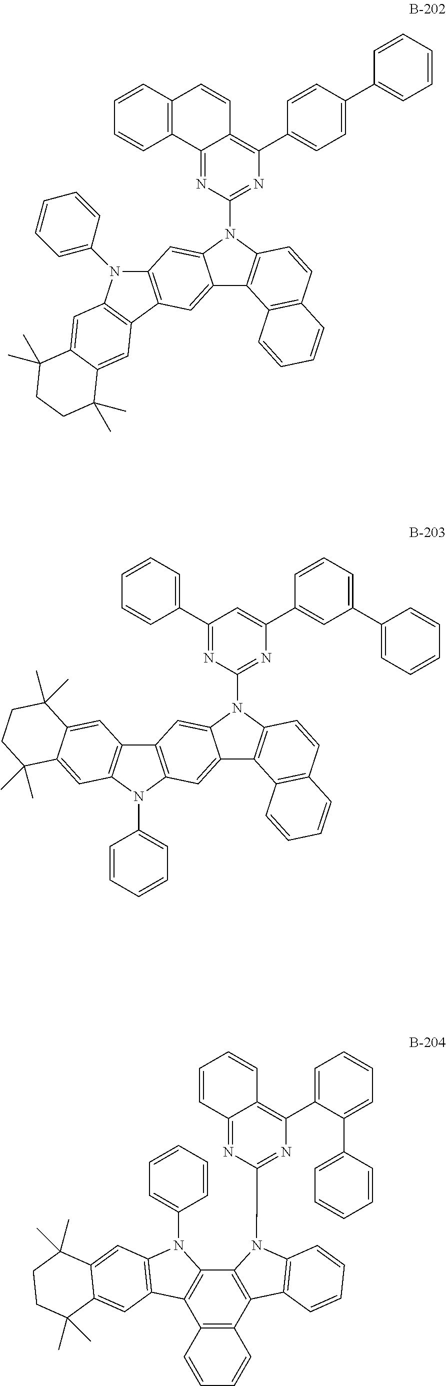 [SMILES: CC1(C)CCC(C)(C)c2cc3c(cc21)c1cc2c4c5ccccc5ccc4n(-c4nc(-c5ccc(-c6ccccc6)cc5)c5ccc6ccccc6c5n4)c2cc1n3-c1ccccc1 ; CC1(C)CCC(C)(C)c2cc3c(cc21)c1cc2c(cc1n3-c1ccccc1)c1c3ccccc3ccc1n2-c1nc(-c2ccccc2)cc(-c2cccc(-c3ccccc3)c2)n1 ; CC1(C)CCC(C)(C)c2cc3c(cc21)c1c2ccccc2c2c4ccccc4n(-c4nc(-c5ccccc5-c5ccccc5)c5ccccc5n4)c2c1n3-c1ccccc1]

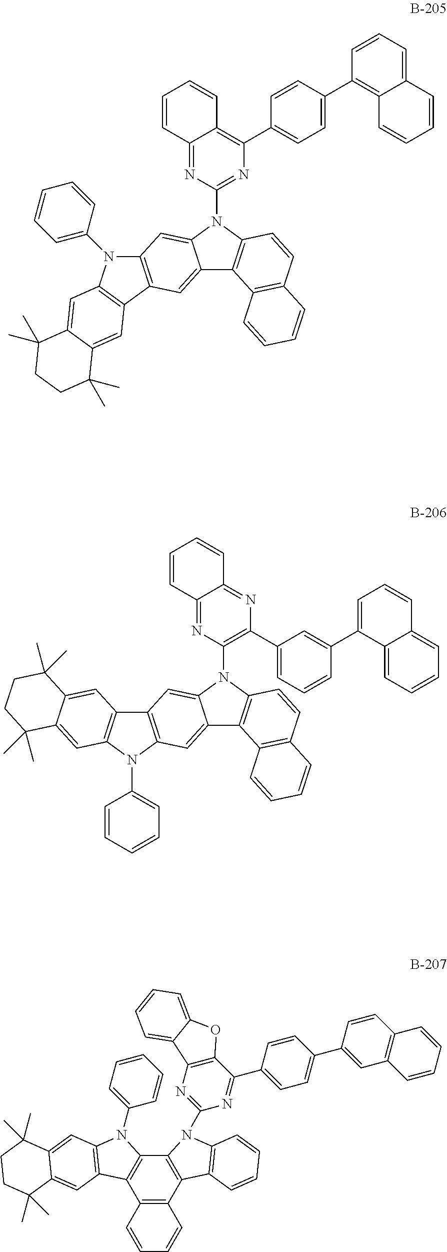 [SMILES: CC1(C)CCC(C)(C)c2cc3c(cc21)c1cc2c4c5ccccc5ccc4n(-c4nc(-c5ccc(-c6cccc7ccccc67)cc5)c5ccccc5n4)c2cc1n3-c1ccccc1 ; CC1(C)CCC(C)(C)c2cc3c(cc21)c1cc2c(cc1n3-c1ccccc1)c1c3ccccc3ccc1n2-c1nc2ccccc2nc1-c1cccc(-c2cccc3ccccc23)c1 ; CC1(C)CCC(C)(C)c2cc3c(cc21)c1c2ccccc2c2c4ccccc4n(-c4nc(-c5ccc(-c6ccc7ccccc7c6)cc5)c5oc6ccccc6c5n4)c2c1n3-c1ccccc1]

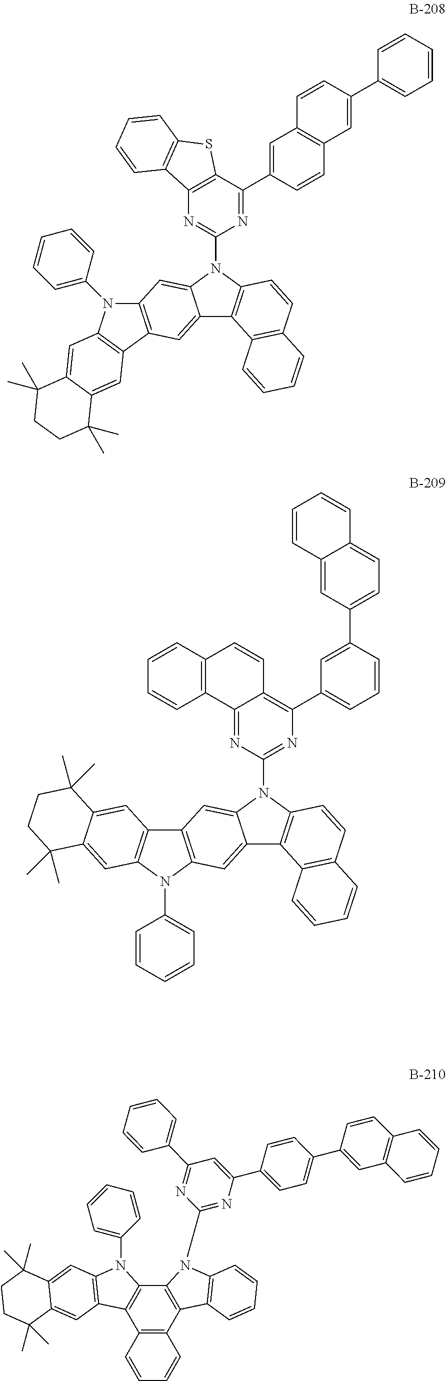 [SMILES: CC1(C)CCC(C)(C)c2cc3c(cc21)c1cc2c4c5ccccc5ccc4n(-c4nc(-c5ccc6cc(-c7ccccc7)ccc6c5)c5sc6ccccc6c5n4)c2cc1n3-c1ccccc1 ; CC1(C)CCC(C)(C)c2cc3c(cc21)c1cc2c(cc1n3-c1ccccc1)c1c3ccccc3ccc1n2-c1nc(-c2cccc(-c3ccc4ccccc4c3)c2)c2ccc3ccccc3c2n1 ; CC1(C)CCC(C)(C)c2cc3c(cc21)c1c2ccccc2c2c4ccccc4n(-c4nc(-c5ccccc5)cc(-c5ccc(-c6ccc7ccccc7c6)cc5)n4)c2c1n3-c1ccccc1]

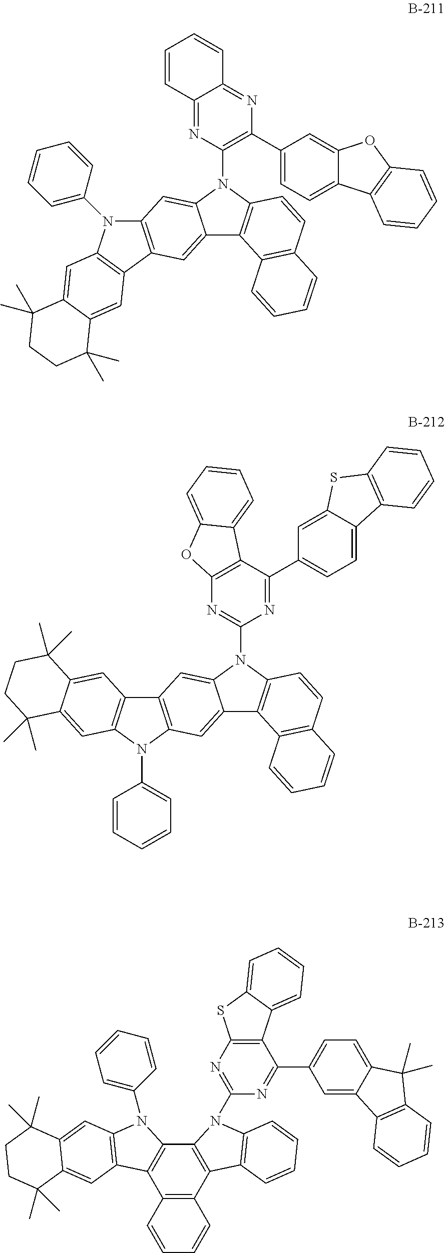 [SMILES: CC1(C)CCC(C)(C)c2cc3c(cc21)c1cc2c4c5ccccc5ccc4n(-c4nc5ccccc5nc4-c4ccc5c(c4)oc4ccccc45)c2cc1n3-c1ccccc1 ; CC1(C)CCC(C)(C)c2cc3c(cc21)c1cc2c(cc1n3-c1ccccc1)c1c3ccccc3ccc1n2-c1nc(-c2ccc3c(c2)sc2ccccc23)c2c(n1)oc1ccccc12 ; CC1(C)CCC(C)(C)c2cc3c(cc21)c1c2ccccc2c2c4ccccc4n(-c4nc(-c5ccc6c(c5)-c5ccccc5C6(C)C)c5c(n4)sc4ccccc45)c2c1n3-c1ccccc1]

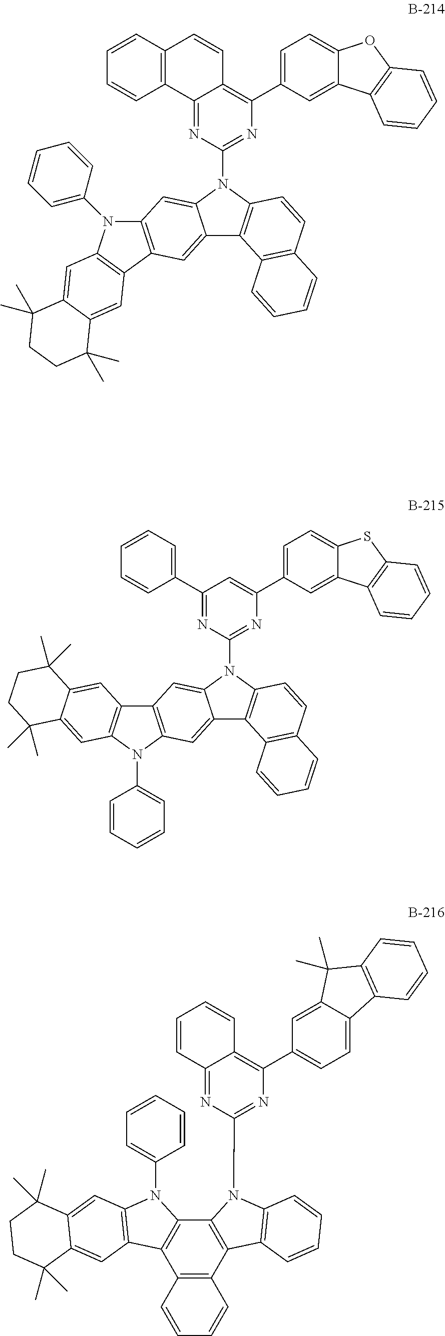 [SMILES: CC1(C)CCC(C)(C)c2cc3c(cc21)c1cc2c4c5ccccc5ccc4n(-c4nc(-c5ccc6oc7ccccc7c6c5)c5ccc6ccccc6c5n4)c2cc1n3-c1ccccc1 ; CC1(C)CCC(C)(C)c2cc3c(cc21)c1cc2c(cc1n3-c1ccccc1)c1c3ccccc3ccc1n2-c1nc(-c2ccccc2)cc(-c2ccc3sc4ccccc4c3c2)n1 ; CC1(C)CCC(C)(C)c2cc3c(cc21)c1c2ccccc2c2c4ccccc4n(-c4nc(-c5ccc6c(c5)C(C)(C)c5ccccc5-6)c5ccccc5n4)c2c1n3-c1ccccc1]

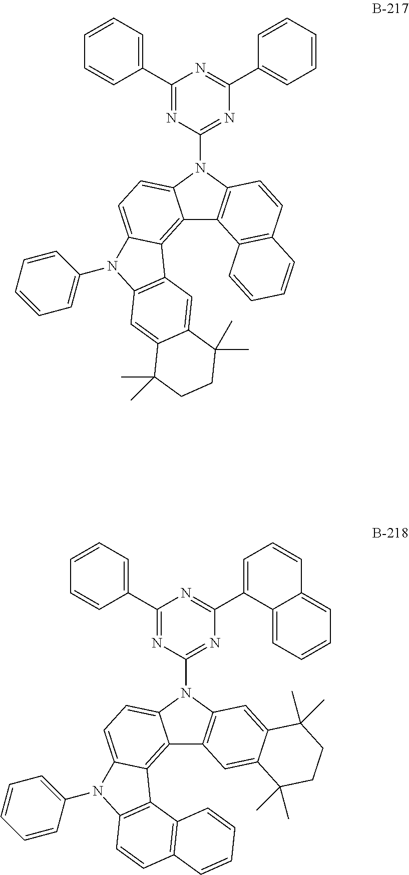 [SMILES: CC1(C)CCC(C)(C)c2cc3c(cc21)c1c2c4c5ccccc5ccc4n(-c4nc(-c5ccccc5)nc(-c5ccccc5)n4)c2ccc1n3-c1ccccc1 ; CC1(C)CCC(C)(C)c2cc3c(cc21)c1c2c4c5ccccc5ccc4n(-c4ccccc4)c2ccc1n3-c1nc(-c2ccccc2)nc(-c2cccc3ccccc23)n1]

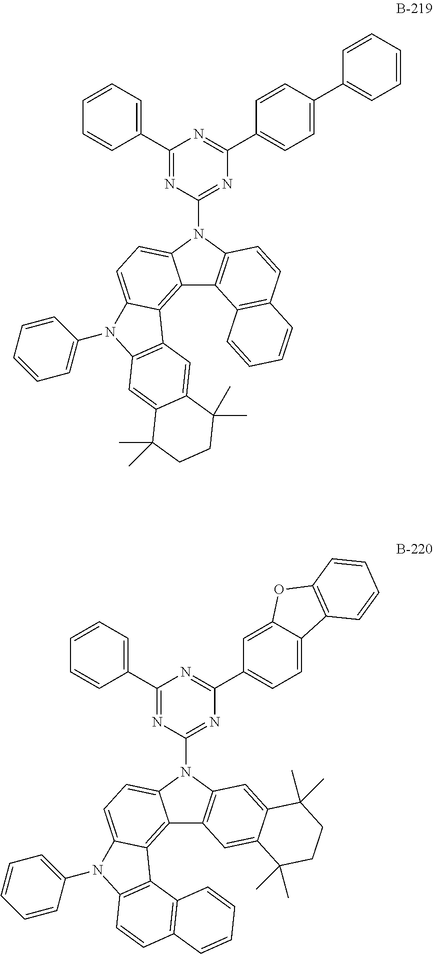 [SMILES: CC1(C)CCC(C)(C)c2cc3c(cc21)c1c2c4c5ccccc5ccc4n(-c4nc(-c5ccccc5)nc(-c5ccc(-c6ccccc6)cc5)n4)c2ccc1n3-c1ccccc1 ; CC1(C)CCC(C)(C)c2cc3c(cc21)c1c2c4c5ccccc5ccc4n(-c4ccccc4)c2ccc1n3-c1nc(-c2ccccc2)nc(-c2ccc3c(c2)oc2ccccc23)n1]

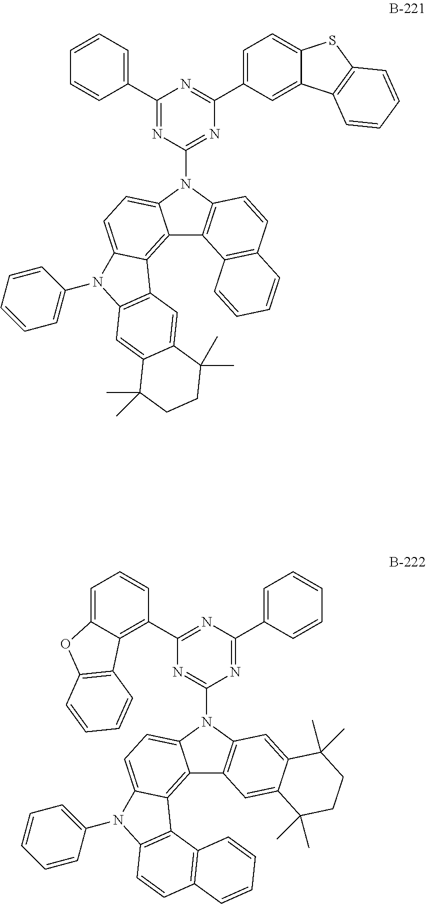 [SMILES: CC1(C)CCC(C)(C)c2cc3c(cc21)c1c2c4c5ccccc5ccc4n(-c4nc(-c5ccccc5)nc(-c5ccc6sc7ccccc7c6c5)n4)c2ccc1n3-c1ccccc1 ; CC1(C)CCC(C)(C)c2cc3c(cc21)c1c2c4c5ccccc5ccc4n(-c4ccccc4)c2ccc1n3-c1nc(-c2ccccc2)nc(-c2cccc3oc4ccccc4c23)n1]

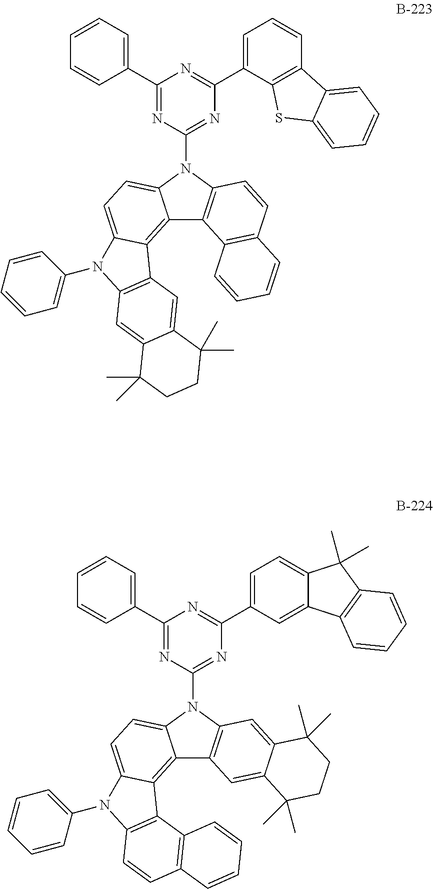 [SMILES: CC1(C)CCC(C)(C)c2cc3c(cc21)c1c2c4c5ccccc5ccc4n(-c4nc(-c5ccccc5)nc(-c5cccc6c5sc5ccccc56)n4)c2ccc1n3-c1ccccc1 ; CC1(C)CCC(C)(C)c2cc3c(cc21)c1c2c4c5ccccc5ccc4n(-c4ccccc4)c2ccc1n3-c1nc(-c2ccccc2)nc(-c2ccc3c(c2)-c2ccccc2C3(C)C)n1]

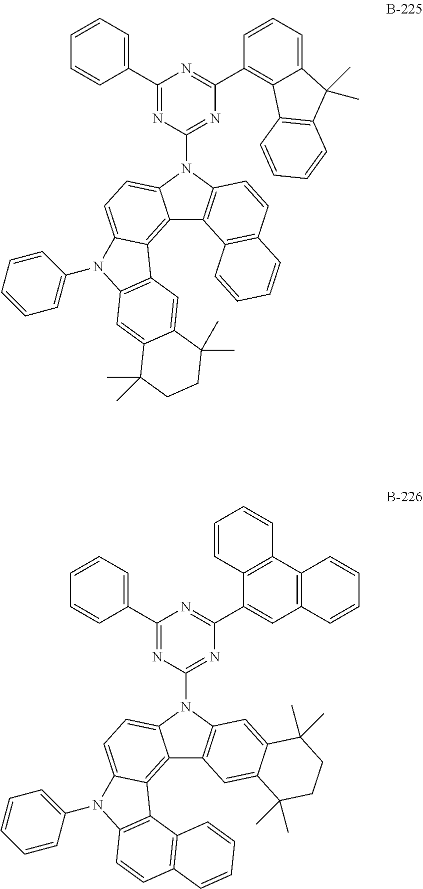 [SMILES: CC1(C)CCC(C)(C)c2cc3c(cc21)c1c2c4c5ccccc5ccc4n(-c4nc(-c5ccccc5)nc(-c5cccc6c5-c5ccccc5C6(C)C)n4)c2ccc1n3-c1ccccc1 ; CC1(C)CCC(C)(C)c2cc3c(cc21)c1c2c4c5ccccc5ccc4n(-c4ccccc4)c2ccc1n3-c1nc(-c2ccccc2)nc(-c2cc3ccccc3c3ccccc23)n1]

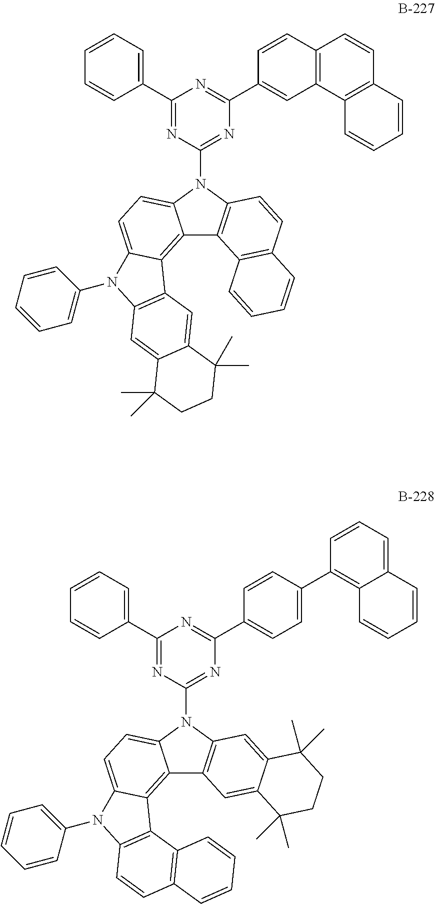 [SMILES: CC1(C)CCC(C)(C)c2cc3c(cc21)c1c2c4c5ccccc5ccc4n(-c4nc(-c5ccccc5)nc(-c5ccc6ccc7ccccc7c6c5)n4)c2ccc1n3-c1ccccc1 ; CC1(C)CCC(C)(C)c2cc3c(cc21)c1c2c4c5ccccc5ccc4n(-c4ccccc4)c2ccc1n3-c1nc(-c2ccccc2)nc(-c2ccc(-c3cccc4ccccc34)cc2)n1]

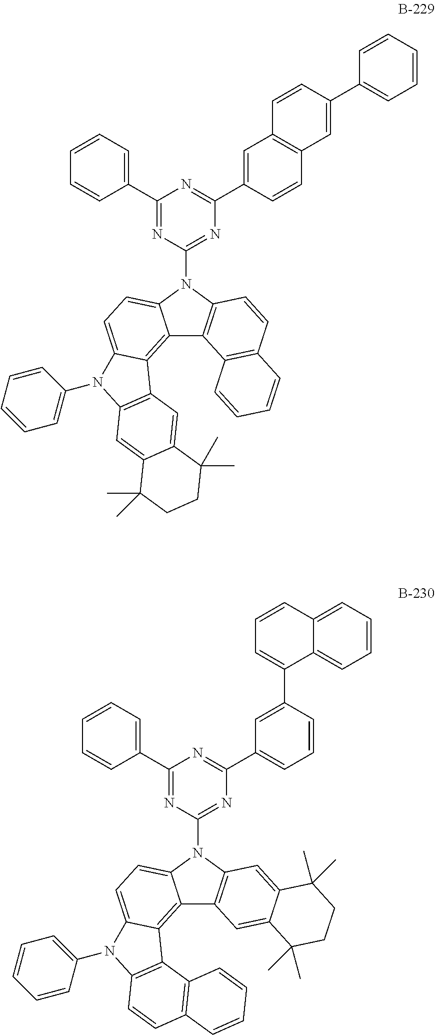 [SMILES: CC1(C)CCC(C)(C)c2cc3c(cc21)c1c2c4c5ccccc5ccc4n(-c4nc(-c5ccccc5)nc(-c5ccc6cc(-c7ccccc7)ccc6c5)n4)c2ccc1n3-c1ccccc1 ; CC1(C)CCC(C)(C)c2cc3c(cc21)c1c2c4c5ccccc5ccc4n(-c4ccccc4)c2ccc1n3-c1nc(-c2ccccc2)nc(-c2cccc(-c3cccc4ccccc34)c2)n1]

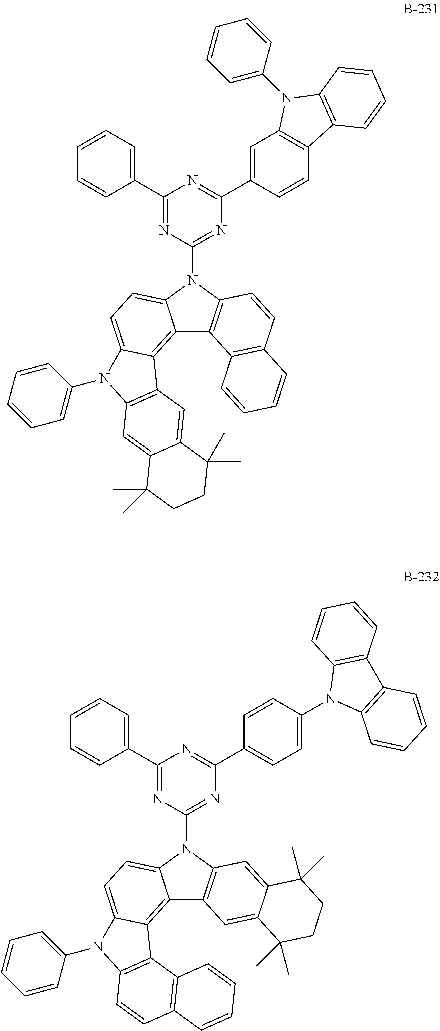 [SMILES: CC1(C)CCC(C)(C)c2cc3c(cc21)c1c2c4c5ccccc5ccc4n(-c4nc(-c5ccccc5)nc(-c5ccc6c7ccccc7n(-c7ccccc7)c6c5)n4)c2ccc1n3-c1ccccc1 ; CC1(C)CCC(C)(C)c2cc3c(cc21)c1c2c4c5ccccc5ccc4n(-c4ccccc4)c2ccc1n3-c1nc(-c2ccccc2)nc(-c2ccc(-n3c4ccccc4c4ccccc43)cc2)n1]

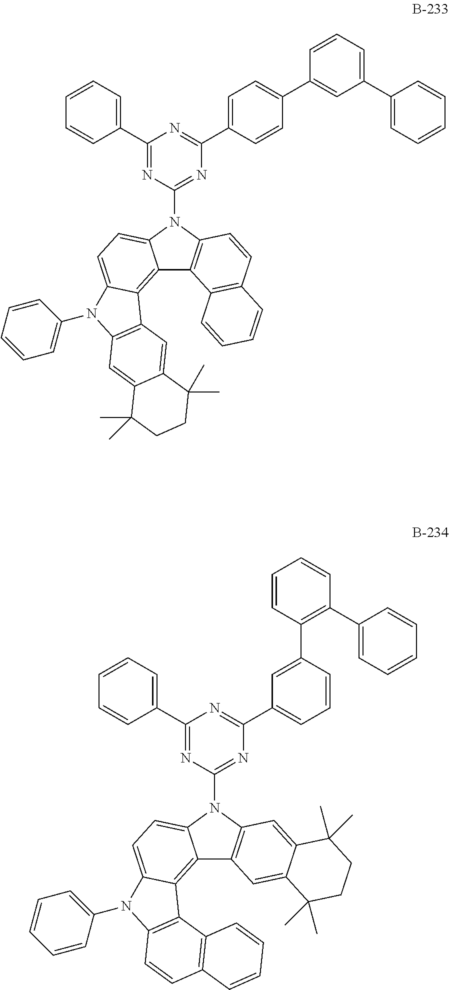 [SMILES: CC1(C)CCC(C)(C)c2cc3c(cc21)c1c2c4c5ccccc5ccc4n(-c4nc(-c5ccccc5)nc(-c5ccc(-c6cccc(-c7ccccc7)c6)cc5)n4)c2ccc1n3-c1ccccc1 ; CC1(C)CCC(C)(C)c2cc3c(cc21)c1c2c4c5ccccc5ccc4n(-c4ccccc4)c2ccc1n3-c1nc(-c2ccccc2)nc(-c2cccc(-c3ccccc3-c3ccccc3)c2)n1]

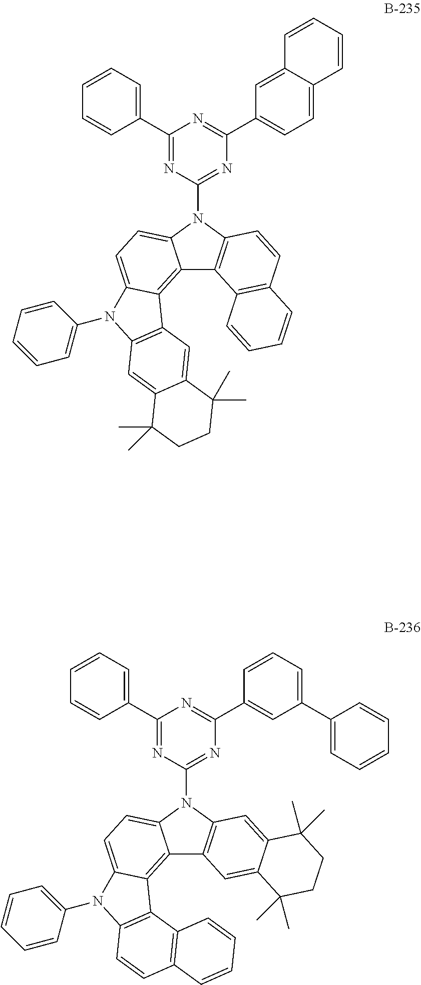 [SMILES: CC1(C)CCC(C)(C)c2cc3c(cc21)c1c2c4c5ccccc5ccc4n(-c4nc(-c5ccccc5)nc(-c5ccc6ccccc6c5)n4)c2ccc1n3-c1ccccc1 ; CC1(C)CCC(C)(C)c2cc3c(cc21)c1c2c4c5ccccc5ccc4n(-c4ccccc4)c2ccc1n3-c1nc(-c2ccccc2)nc(-c2cccc(-c3ccccc3)c2)n1]

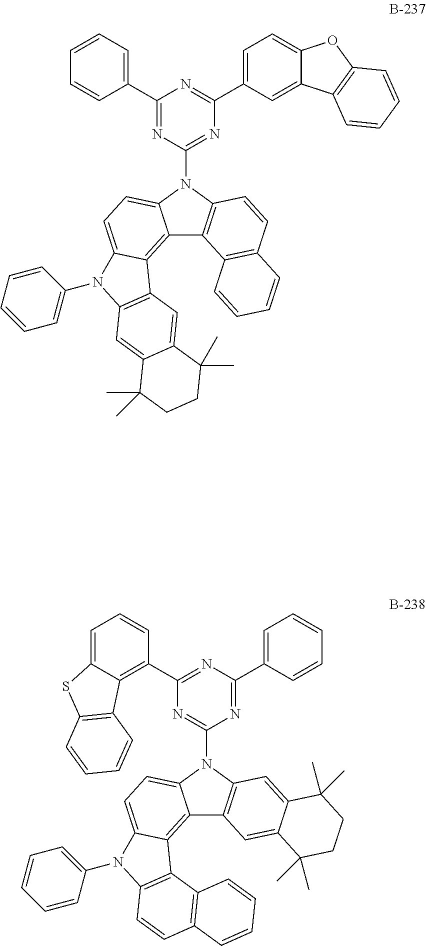 [SMILES: CC1(C)CCC(C)(C)c2cc3c(cc21)c1c2c4c5ccccc5ccc4n(-c4nc(-c5ccccc5)nc(-c5ccc6oc7ccccc7c6c5)n4)c2ccc1n3-c1ccccc1 ; CC1(C)CCC(C)(C)c2cc3c(cc21)c1c2c4c5ccccc5ccc4n(-c4ccccc4)c2ccc1n3-c1nc(-c2ccccc2)nc(-c2cccc3sc4ccccc4c23)n1]

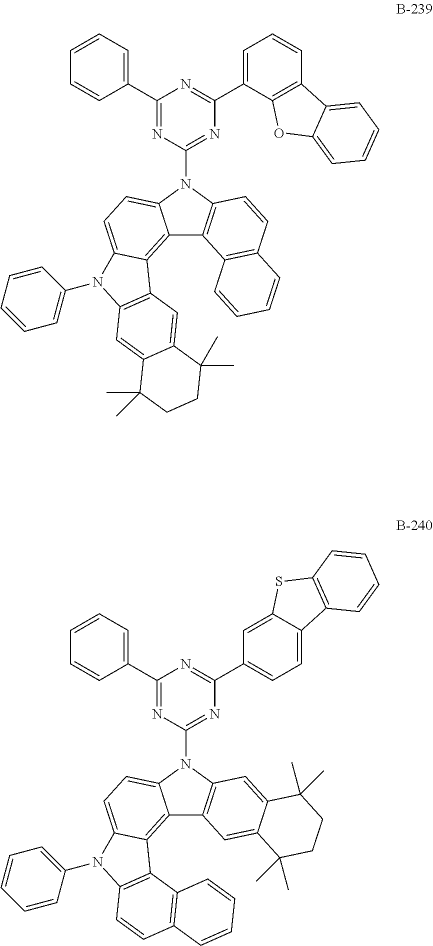 [SMILES: CC1(C)CCC(C)(C)c2cc3c(cc21)c1c2c4c5ccccc5ccc4n(-c4nc(-c5ccccc5)nc(-c5cccc6c5oc5ccccc56)n4)c2ccc1n3-c1ccccc1 ; CC1(C)CCC(C)(C)c2cc3c(cc21)c1c2c4c5ccccc5ccc4n(-c4ccccc4)c2ccc1n3-c1nc(-c2ccccc2)nc(-c2ccc3c(c2)sc2ccccc23)n1]

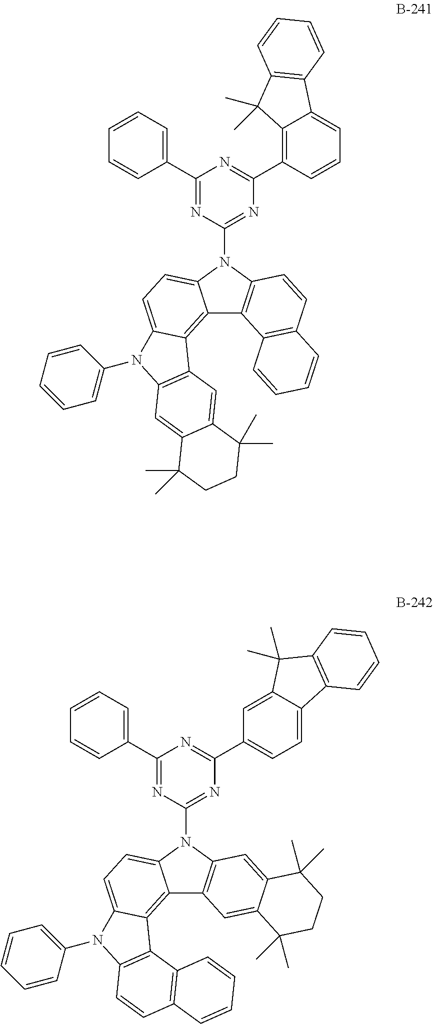 [SMILES: CC1(C)CCC(C)(C)c2cc3c(cc21)c1c2c4c5ccccc5ccc4n(-c4nc(-c5ccccc5)nc(-c5cccc6c5C(C)(C)c5ccccc5-6)n4)c2ccc1n3-c1ccccc1 ; CC1(C)CCC(C)(C)c2cc3c(cc21)c1c2c4c5ccccc5ccc4n(-c4ccccc4)c2ccc1n3-c1nc(-c2ccccc2)nc(-c2ccc3c(c2)C(C)(C)c2ccccc2-3)n1]

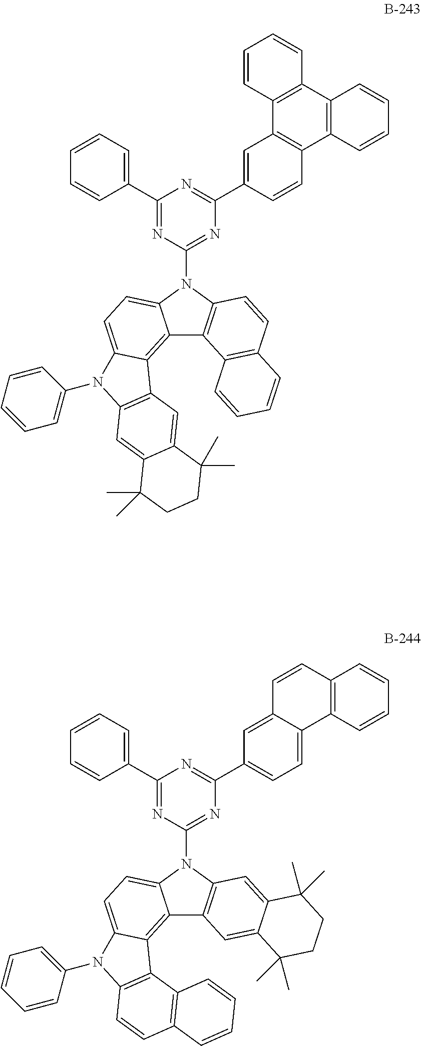 [SMILES: CC1(C)CCC(C)(C)c2cc3c(cc21)c1c2c4c5ccccc5ccc4n(-c4nc(-c5ccccc5)nc(-c5ccc6c7ccccc7c7ccccc7c6c5)n4)c2ccc1n3-c1ccccc1 ; CC1(C)CCC(C)(C)c2cc3c(cc21)c1c2c4c5ccccc5ccc4n(-c4ccccc4)c2ccc1n3-c1nc(-c2ccccc2)nc(-c2ccc3c(ccc4ccccc43)c2)n1]

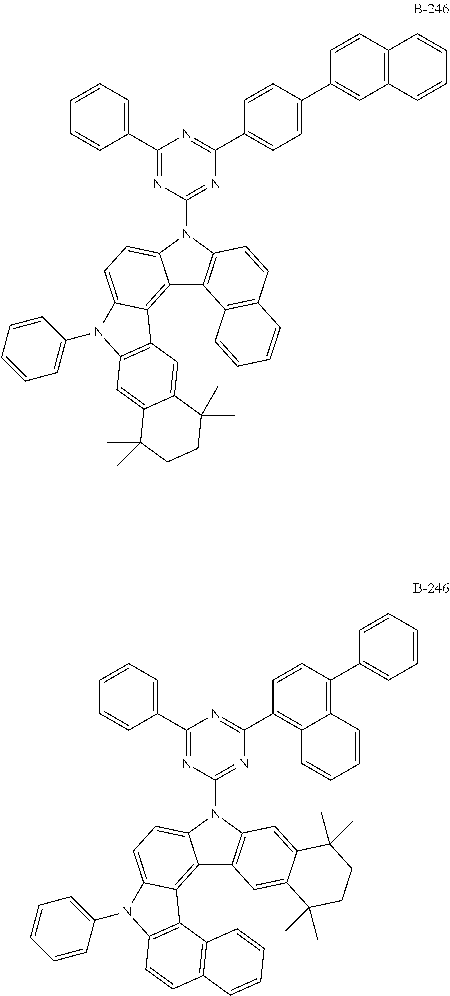 [SMILES: CC1(C)CCC(C)(C)c2cc3c(cc21)c1c2c4c5ccccc5ccc4n(-c4nc(-c5ccccc5)nc(-c5ccc(-c6ccc7ccccc7c6)cc5)n4)c2ccc1n3-c1ccccc1 ; CC1(C)CCC(C)(C)c2cc3c(cc21)c1c2c4c5ccccc5ccc4n(-c4ccccc4)c2ccc1n3-c1nc(-c2ccccc2)nc(-c2ccc(-c3ccccc3)c3ccccc23)n1]

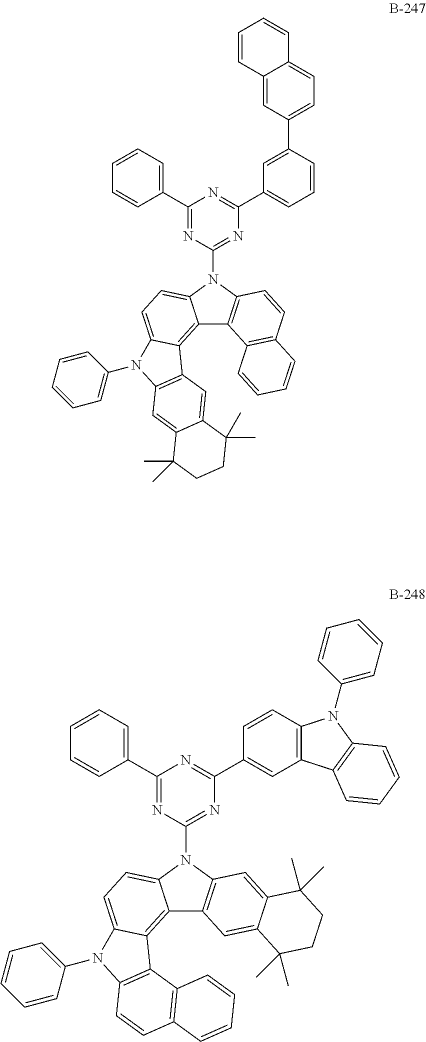 [SMILES: CC1(C)CCC(C)(C)c2cc3c(cc21)c1c2c4c5ccccc5ccc4n(-c4nc(-c5ccccc5)nc(-c5cccc(-c6ccc7ccccc7c6)c5)n4)c2ccc1n3-c1ccccc1 ; CC1(C)CCC(C)(C)c2cc3c(cc21)c1c2c4c5ccccc5ccc4n(-c4ccccc4)c2ccc1n3-c1nc(-c2ccccc2)nc(-c2ccc3c(c2)c2ccccc2n3-c2ccccc2)n1]

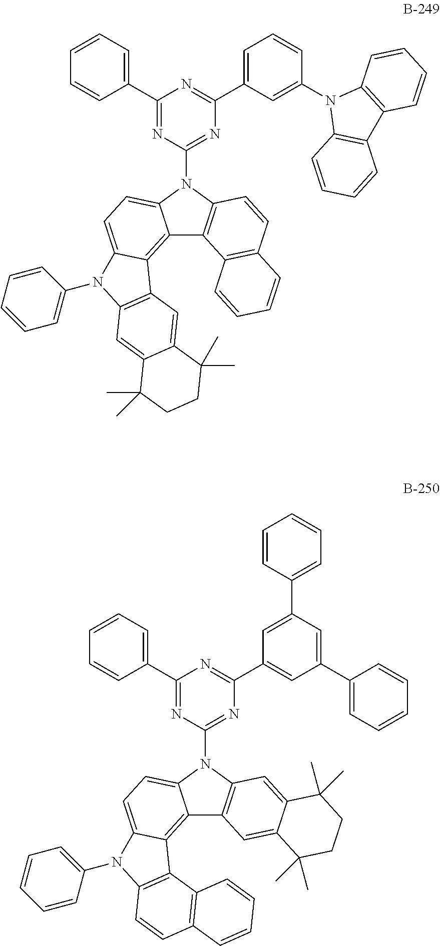 [SMILES: CC1(C)CCC(C)(C)c2cc3c(cc21)c1c2c4c5ccccc5ccc4n(-c4nc(-c5ccccc5)nc(-c5cccc(-n6c7ccccc7c7ccccc76)c5)n4)c2ccc1n3-c1ccccc1 ; CC1(C)CCC(C)(C)c2cc3c(cc21)c1c2c4c5ccccc5ccc4n(-c4ccccc4)c2ccc1n3-c1nc(-c2ccccc2)nc(-c2cc(-c3ccccc3)cc(-c3ccccc3)c2)n1]

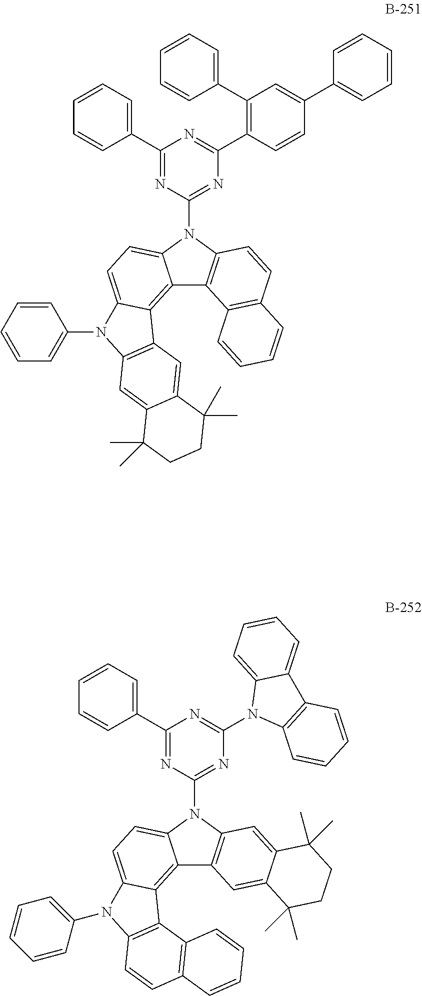 [SMILES: CC1(C)CCC(C)(C)c2cc3c(cc21)c1c2c4c5ccccc5ccc4n(-c4nc(-c5ccccc5)nc(-c5ccc(-c6ccccc6)cc5-c5ccccc5)n4)c2ccc1n3-c1ccccc1 ; CC1(C)CCC(C)(C)c2cc3c(cc21)c1c2c4c5ccccc5ccc4n(-c4ccccc4)c2ccc1n3-c1nc(-c2ccccc2)nc(-n2c3ccccc3c3ccccc32)n1]

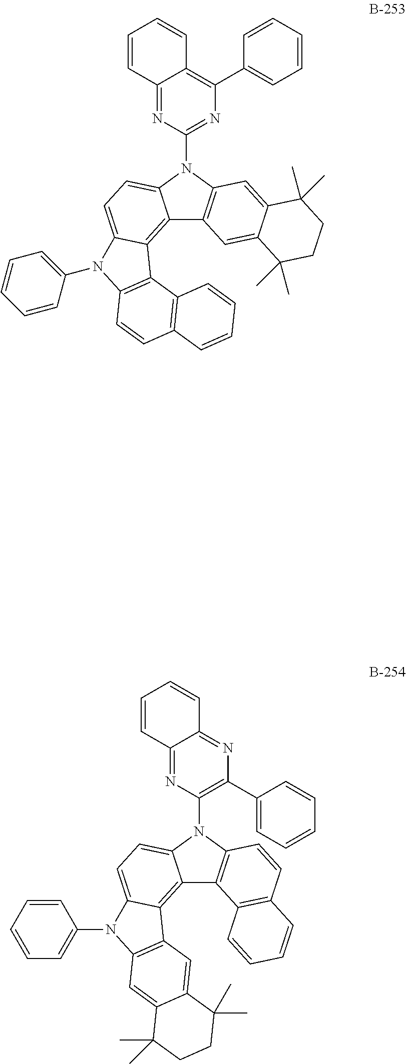 [SMILES: CC1(C)CCC(C)(C)c2cc3c(cc21)c1c2c4c5ccccc5ccc4n(-c4ccccc4)c2ccc1n3-c1nc(-c2ccccc2)c2ccccc2n1 ; CC1(C)CCC(C)(C)c2cc3c(cc21)c1c2c4c5ccccc5ccc4n(-c4nc5ccccc5nc4-c4ccccc4)c2ccc1n3-c1ccccc1]

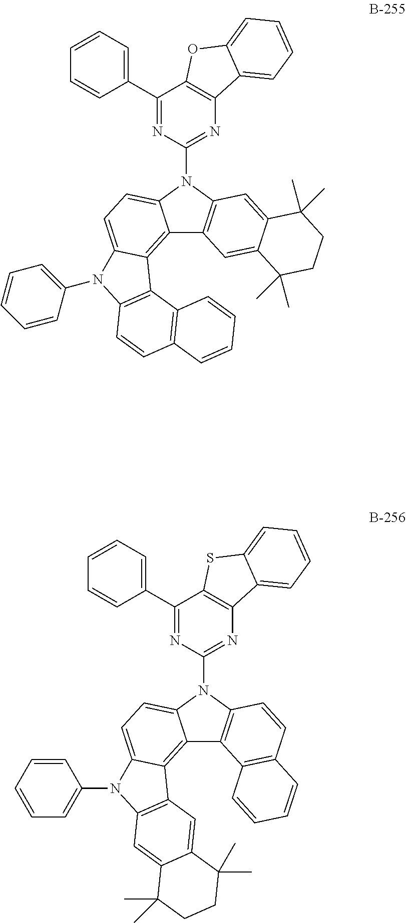 [SMILES: CC1(C)CCC(C)(C)c2cc3c(cc21)c1c2c4c5ccccc5ccc4n(-c4ccccc4)c2ccc1n3-c1nc(-c2ccccc2)c2oc3ccccc3c2n1 ; CC1(C)CCC(C)(C)c2cc3c(cc21)c1c2c4c5ccccc5ccc4n(-c4nc(-c5ccccc5)c5sc6ccccc6c5n4)c2ccc1n3-c1ccccc1]

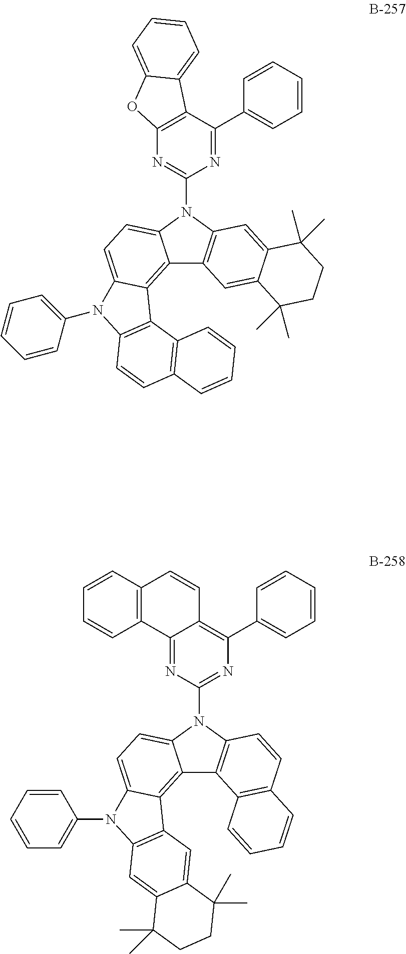 [SMILES: CC1(C)CCC(C)(C)c2cc3c(cc21)c1c2c4c5ccccc5ccc4n(-c4ccccc4)c2ccc1n3-c1nc(-c2ccccc2)c2c(n1)oc1ccccc12 ; CC1(C)CCC(C)(C)c2cc3c(cc21)c1c2c4c5ccccc5ccc4n(-c4nc(-c5ccccc5)c5ccc6ccccc6c5n4)c2ccc1n3-c1ccccc1]

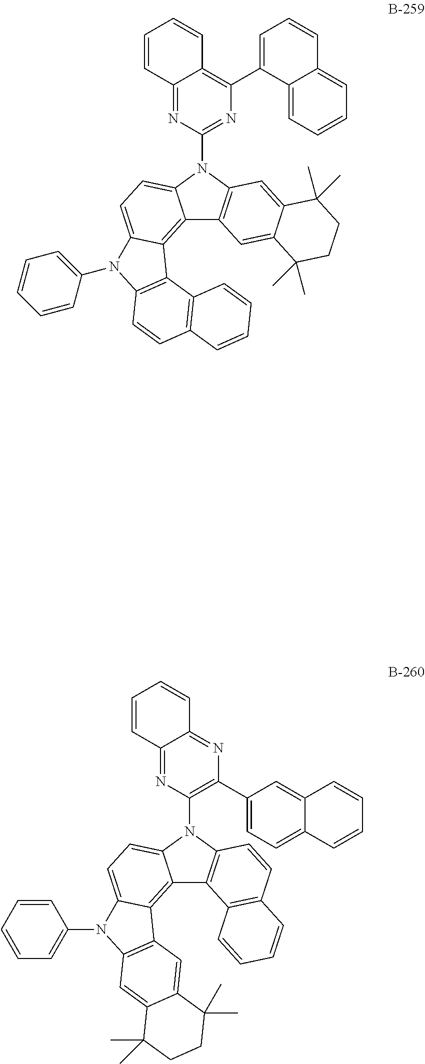 [SMILES: CC1(C)CCC(C)(C)c2cc3c(cc21)c1c2c4c5ccccc5ccc4n(-c4ccccc4)c2ccc1n3-c1nc(-c2cccc3ccccc23)c2ccccc2n1 ; CC1(C)CCC(C)(C)c2cc3c(cc21)c1c2c4c5ccccc5ccc4n(-c4nc5ccccc5nc4-c4ccc5ccccc5c4)c2ccc1n3-c1ccccc1]

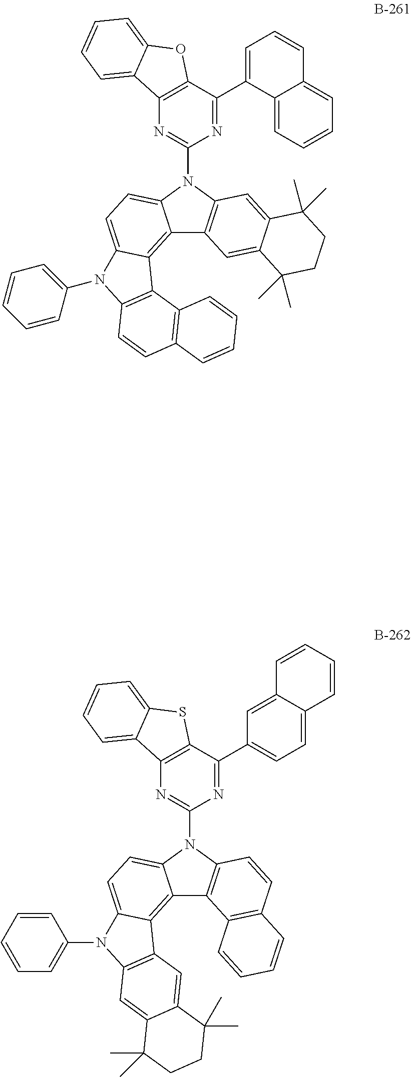 [SMILES: CC1(C)CCC(C)(C)c2cc3c(cc21)c1c2c4c5ccccc5ccc4n(-c4ccccc4)c2ccc1n3-c1nc(-c2cccc3ccccc23)c2oc3ccccc3c2n1 ; CC1(C)CCC(C)(C)c2cc3c(cc21)c1c2c4c5ccccc5ccc4n(-c4nc(-c5ccc6ccccc6c5)c5sc6ccccc6c5n4)c2ccc1n3-c1ccccc1]

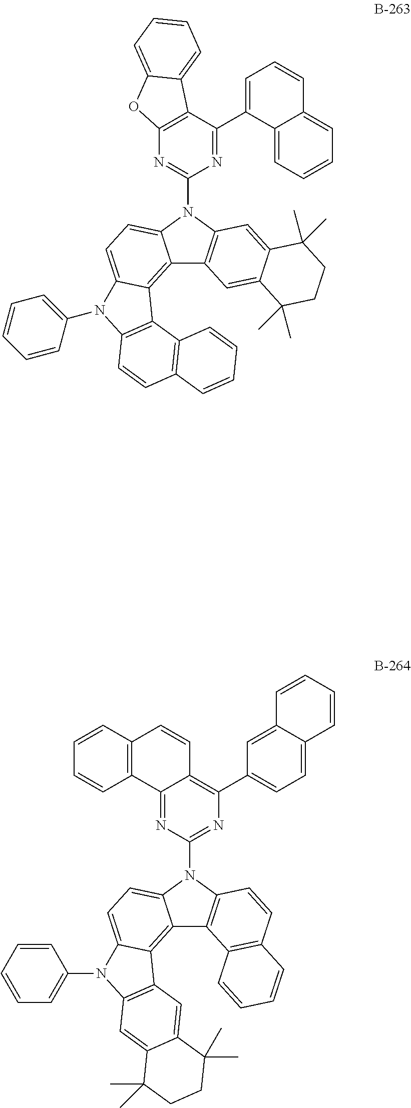 [SMILES: CC1(C)CCC(C)(C)c2cc3c(cc21)c1c2c4c5ccccc5ccc4n(-c4ccccc4)c2ccc1n3-c1nc(-c2cccc3ccccc23)c2c(n1)oc1ccccc12 ; CC1(C)CCC(C)(C)c2cc3c(cc21)c1c2c4c5ccccc5ccc4n(-c4nc(-c5ccc6ccccc6c5)c5ccc6ccccc6c5n4)c2ccc1n3-c1ccccc1]

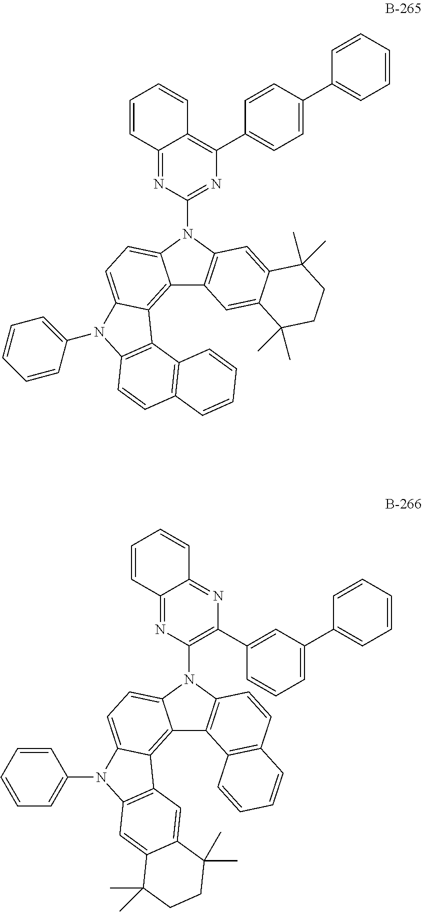 [SMILES: CC1(C)CCC(C)(C)c2cc3c(cc21)c1c2c4c5ccccc5ccc4n(-c4ccccc4)c2ccc1n3-c1nc(-c2ccc(-c3ccccc3)cc2)c2ccccc2n1 ; CC1(C)CCC(C)(C)c2cc3c(cc21)c1c2c4c5ccccc5ccc4n(-c4nc5ccccc5nc4-c4cccc(-c5ccccc5)c4)c2ccc1n3-c1ccccc1]

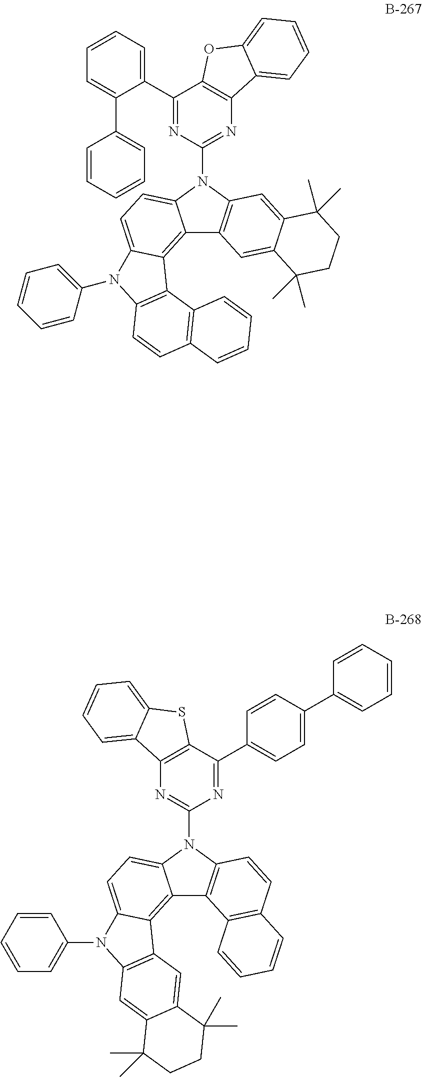 [SMILES: CC1(C)CCC(C)(C)c2cc3c(cc21)c1c2c4c5ccccc5ccc4n(-c4ccccc4)c2ccc1n3-c1nc(-c2ccccc2-c2ccccc2)c2oc3ccccc3c2n1 ; CC1(C)CCC(C)(C)c2cc3c(cc21)c1c2c4c5ccccc5ccc4n(-c4nc(-c5ccc(-c6ccccc6)cc5)c5sc6ccccc6c5n4)c2ccc1n3-c1ccccc1]

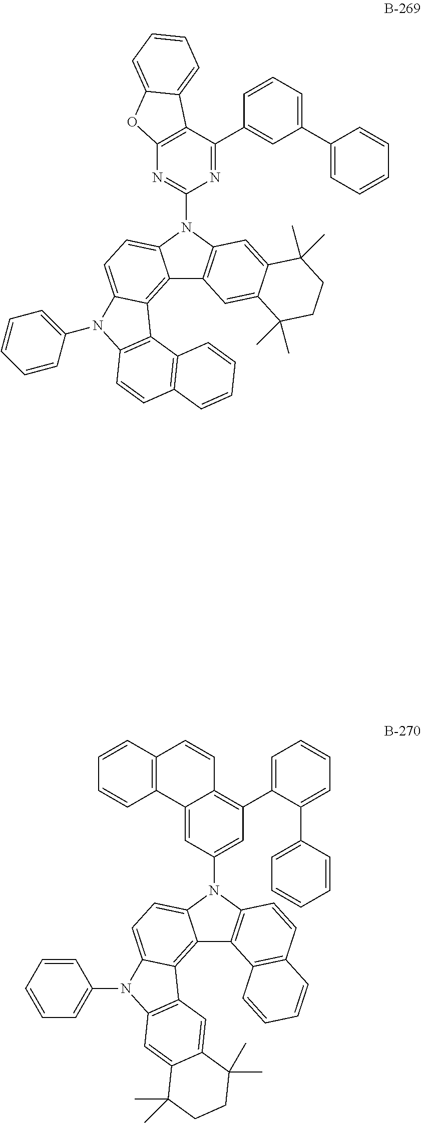 [SMILES: CC1(C)CCC(C)(C)c2cc3c(cc21)c1c2c4c5ccccc5ccc4n(-c4ccccc4)c2ccc1n3-c1nc(-c2cccc(-c3ccccc3)c2)c2c(n1)oc1ccccc12 ; CC1(C)CCC(C)(C)c2cc3c(cc21)c1c2c4c5ccccc5ccc4n(-c4cc(-c5ccccc5-c5ccccc5)c5ccc6ccccc6c5c4)c2ccc1n3-c1ccccc1]

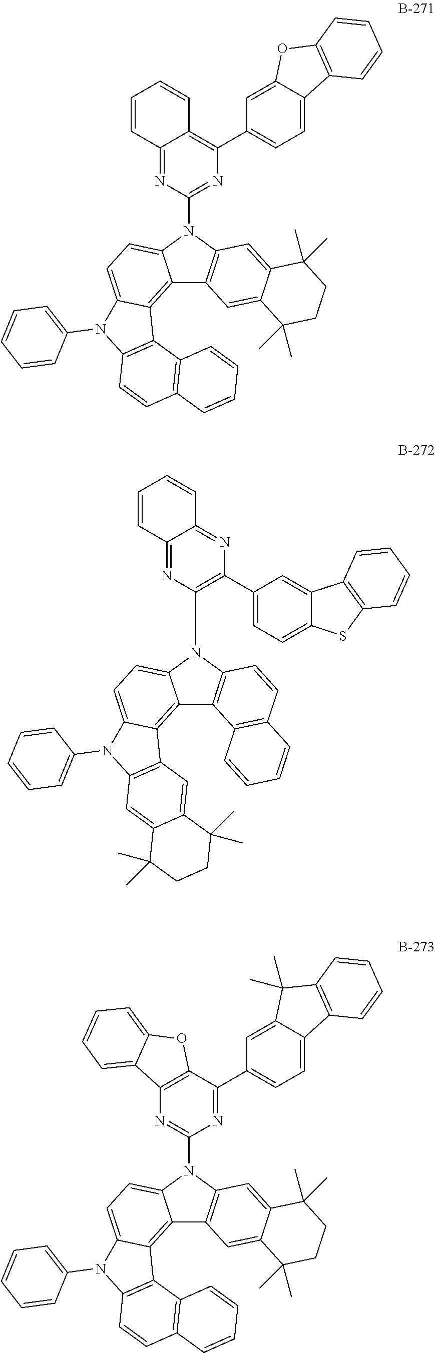 [SMILES: CC1(C)CCC(C)(C)c2cc3c(cc21)c1c2c4c5ccccc5ccc4n(-c4ccccc4)c2ccc1n3-c1nc(-c2ccc3c(c2)oc2ccccc23)c2ccccc2n1 ; CC1(C)CCC(C)(C)c2cc3c(cc21)c1c2c4c5ccccc5ccc4n(-c4nc5ccccc5nc4-c4ccc5sc6ccccc6c5c4)c2ccc1n3-c1ccccc1 ; CC1(C)CCC(C)(C)c2cc3c(cc21)c1c2c4c5ccccc5ccc4n(-c4ccccc4)c2ccc1n3-c1nc(-c2ccc3c(c2)C(C)(C)c2ccccc2-3)c2oc3ccccc3c2n1]

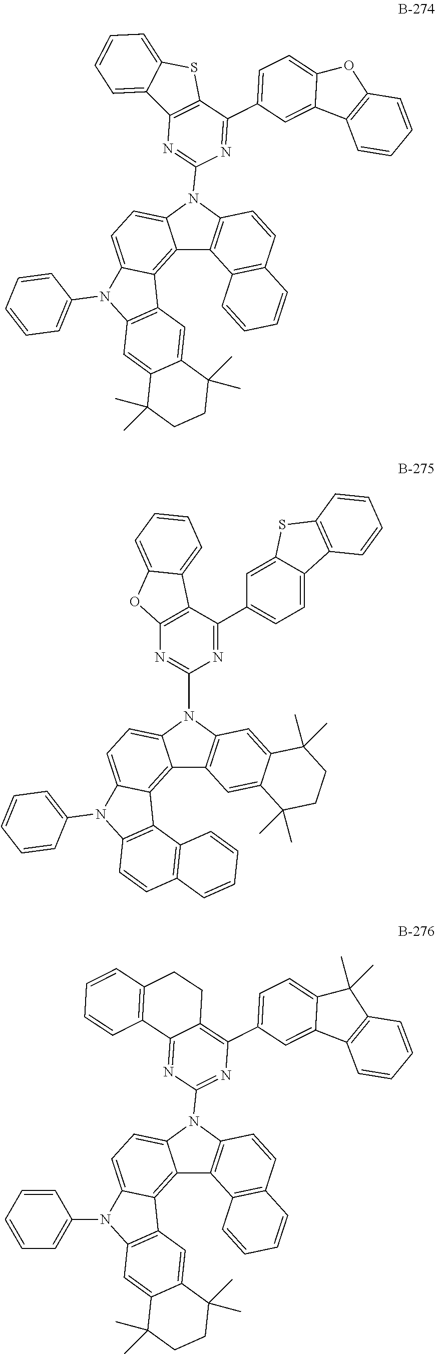 [SMILES: CC1(C)CCC(C)(C)c2cc3c(cc21)c1c2c4c5ccccc5ccc4n(-c4nc(-c5ccc6oc7ccccc7c6c5)c5sc6ccccc6c5n4)c2ccc1n3-c1ccccc1 ; CC1(C)CCC(C)(C)c2cc3c(cc21)c1c2c4c5ccccc5ccc4n(-c4ccccc4)c2ccc1n3-c1nc(-c2ccc3c(c2)sc2ccccc23)c2c(n1)oc1ccccc12 ; CC1(C)CCC(C)(C)c2cc3c(cc21)c1c2c4c5ccccc5ccc4n(-c4nc(-c5ccc6c(c5)-c5ccccc5C6(C)C)c5c(n4)-c4ccccc4CC5)c2ccc1n3-c1ccccc1]

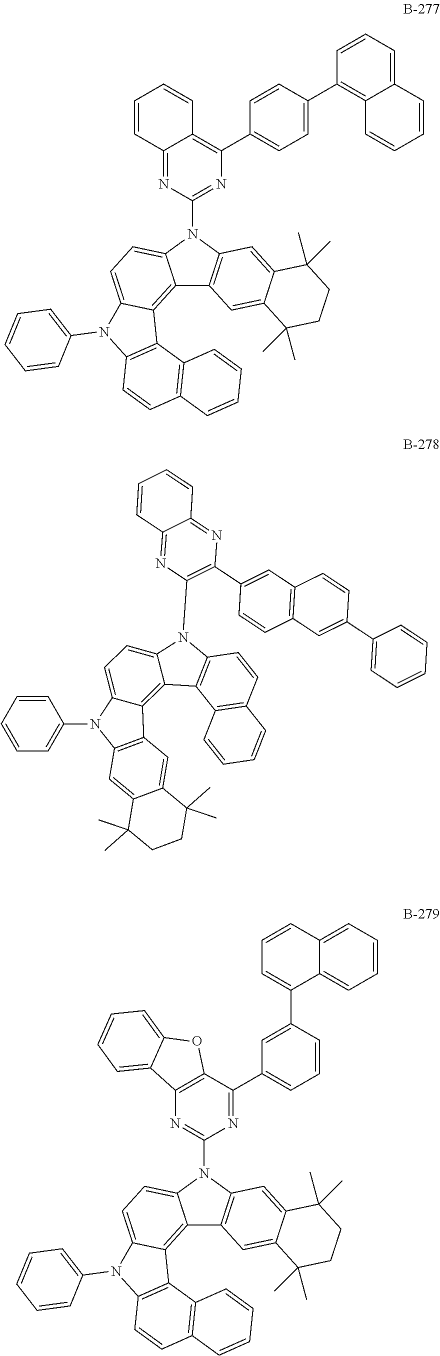 [SMILES: CC1(C)CCC(C)(C)c2cc3c(cc21)c1c2c4c5ccccc5ccc4n(-c4ccccc4)c2ccc1n3-c1nc(-c2ccc(-c3cccc4ccccc34)cc2)c2ccccc2n1 ; CC1(C)CCC(C)(C)c2cc3c(cc21)c1c2c4c5ccccc5ccc4n(-c4nc5ccccc5nc4-c4ccc5cc(-c6ccccc6)ccc5c4)c2ccc1n3-c1ccccc1 ; CC1(C)CCC(C)(C)c2cc3c(cc21)c1c2c4c5ccccc5ccc4n(-c4ccccc4)c2ccc1n3-c1nc(-c2cccc(-c3cccc4ccccc34)c2)c2oc3ccccc3c2n1]

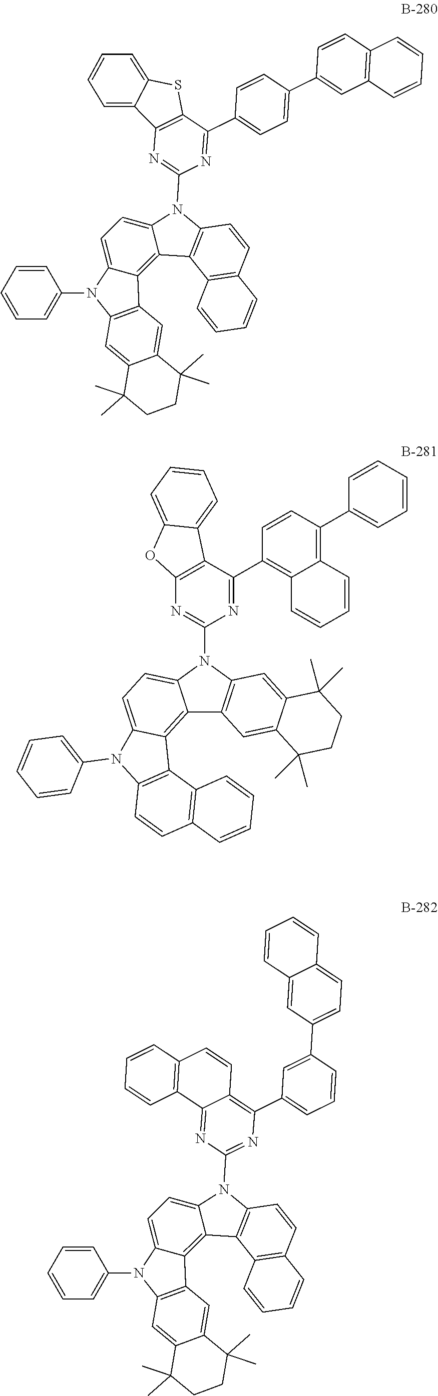 [SMILES: CC1(C)CCC(C)(C)c2cc3c(cc21)c1c2c4c5ccccc5ccc4n(-c4nc(-c5ccc(-c6ccc7ccccc7c6)cc5)c5sc6ccccc6c5n4)c2ccc1n3-c1ccccc1 ; CC1(C)CCC(C)(C)c2cc3c(cc21)c1c2c4c5ccccc5ccc4n(-c4ccccc4)c2ccc1n3-c1nc(-c2ccc(-c3ccccc3)c3ccccc23)c2c(n1)oc1ccccc12 ; CC1(C)CCC(C)(C)c2cc3c(cc21)c1c2c4c5ccccc5ccc4n(-c4nc(-c5cccc(-c6ccc7ccccc7c6)c5)c5ccc6ccccc6c5n4)c2ccc1n3-c1ccccc1]

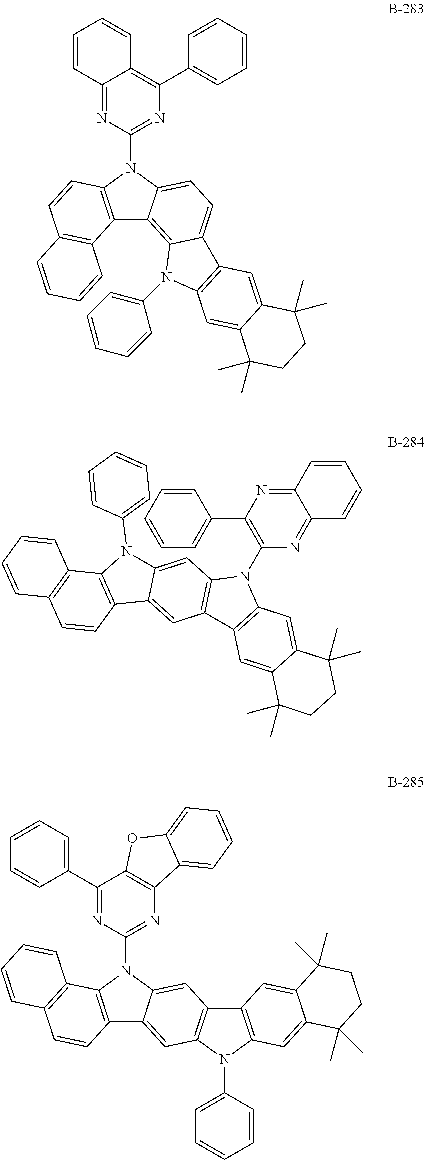 [SMILES: CC1(C)CCC(C)(C)c2cc3c(cc21)c1ccc2c(c4c5ccccc5ccc4n2-c2nc(-c4ccccc4)c4ccccc4n2)c1n3-c1ccccc1 ; CC1(C)CCC(C)(C)c2cc3c(cc21)c1cc2c4ccc5ccccc5c4n(-c4ccccc4)c2cc1n3-c1nc2ccccc2nc1-c1ccccc1 ; CC1(C)CCC(C)(C)c2cc3c(cc21)c1cc2c(cc1n3-c1ccccc1)c1ccc3ccccc3c1n2-c1nc(-c2ccccc2)c2oc3ccccc3c2n1]

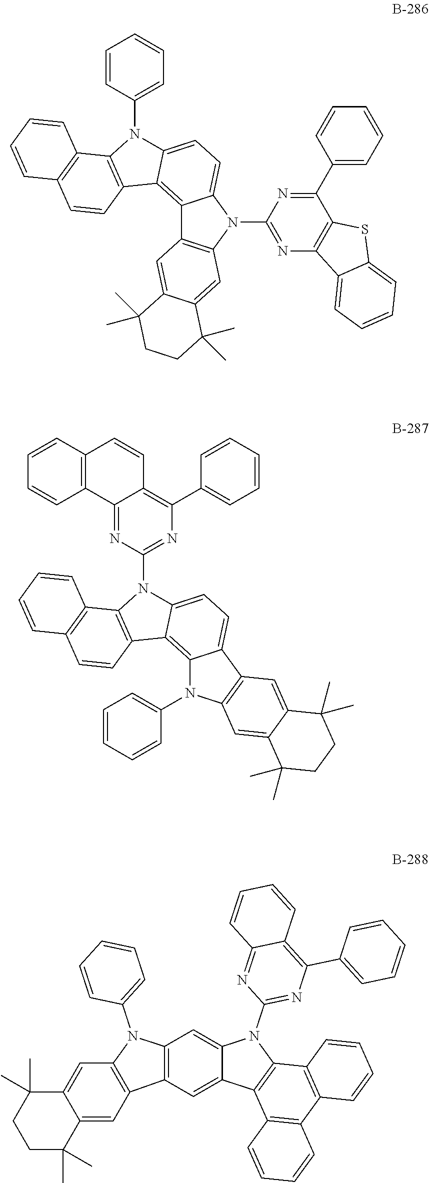

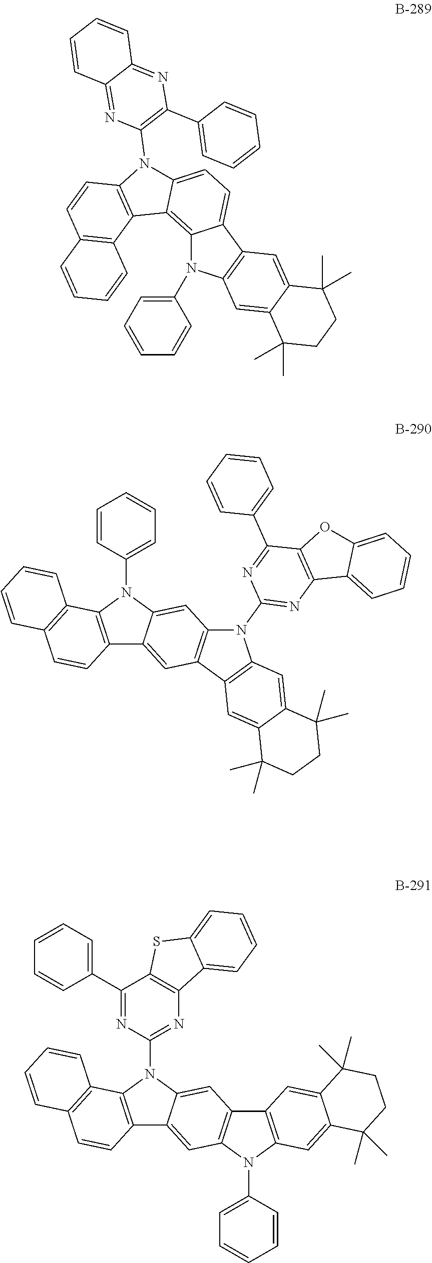 [SMILES: CC1(C)CCC(C)(C)c2cc3c(cc21)c1ccc2c(c4c5ccccc5ccc4n2-c2nc4ccccc4nc2-c2ccccc2)c1n3-c1ccccc1 ; CC1(C)CCC(C)(C)c2cc3c(cc21)c1cc2c4ccc5ccccc5c4n(-c4ccccc4)c2cc1n3-c1nc(-c2ccccc2)c2oc3ccccc3c2n1 ; CC1(C)CCC(C)(C)c2cc3c(cc21)c1cc2c(cc1n3-c1ccccc1)c1ccc3ccccc3c1n2-c1nc(-c2ccccc2)c2sc3ccccc3c2n1]

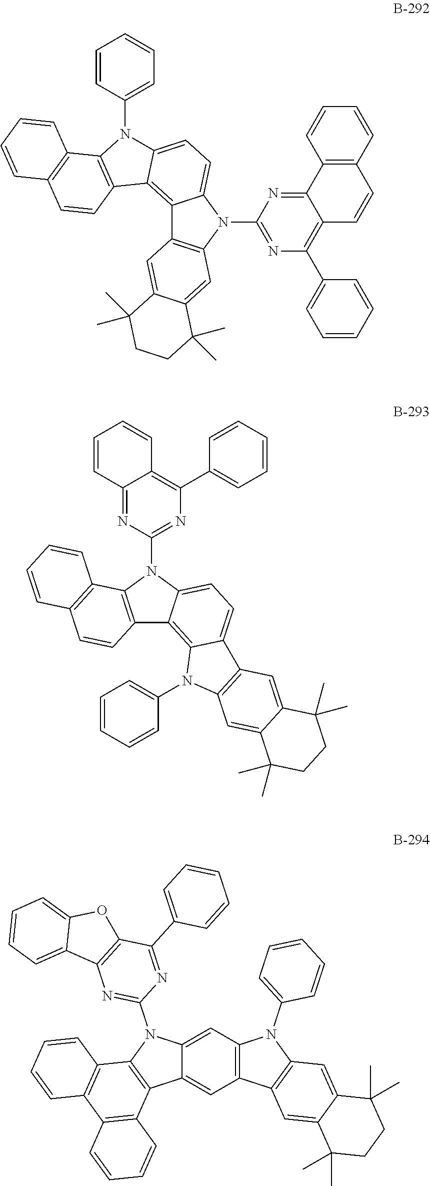 [SMILES: CC1(C)CCC(C)(C)c2cc3c(cc21)c1c2c4ccc5ccccc5c4n(-c4ccccc4)c2ccc1n3-c1nc(-c2ccccc2)c2ccc3ccccc3c2n1 ; CC1(C)CCC(C)(C)c2cc3c(cc21)c1ccc2c(c4ccc5ccccc5c4n2-c2nc(-c4ccccc4)c4ccccc4n2)c1n3-c1ccccc1 ; CC1(C)CCC(C)(C)c2cc3c(cc21)c1cc2c4c5ccccc5c5ccccc5c4n(-c4nc(-c5ccccc5)c5oc6ccccc6c5n4)c2cc1n3-c1ccccc1]

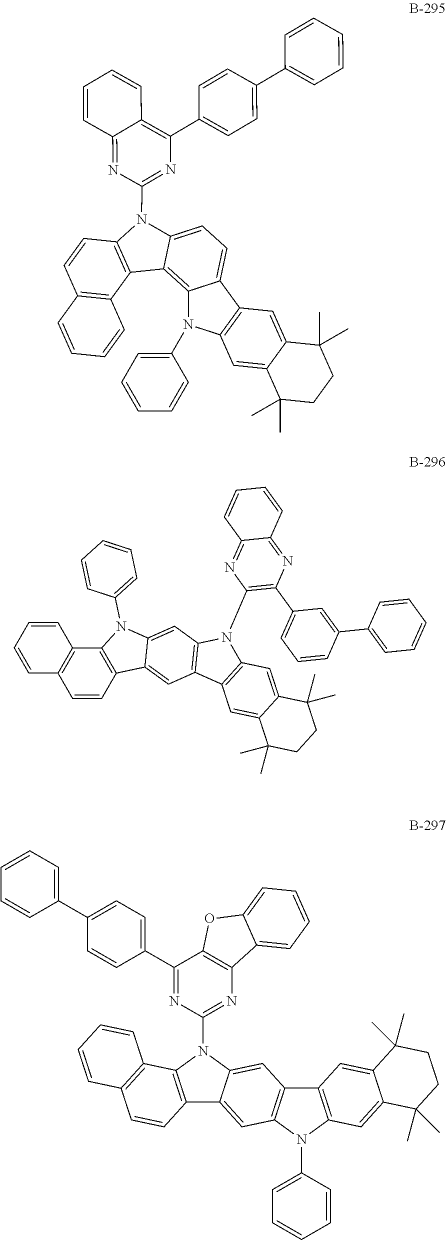 [SMILES: CC1(C)CCC(C)(C)c2cc3c(cc21)c1ccc2c(c4c5ccccc5ccc4n2-c2nc(-c4ccc(-c5ccccc5)cc4)c4ccccc4n2)c1n3-c1ccccc1 ; CC1(C)CCC(C)(C)c2cc3c(cc21)c1cc2c4ccc5ccccc5c4n(-c4ccccc4)c2cc1n3-c1nc2ccccc2nc1-c1cccc(-c2ccccc2)c1 ; CC1(C)CCC(C)(C)c2cc3c(cc21)c1cc2c(cc1n3-c1ccccc1)c1ccc3ccccc3c1n2-c1nc(-c2ccc(-c3ccccc3)cc2)c2oc3ccccc3c2n1]

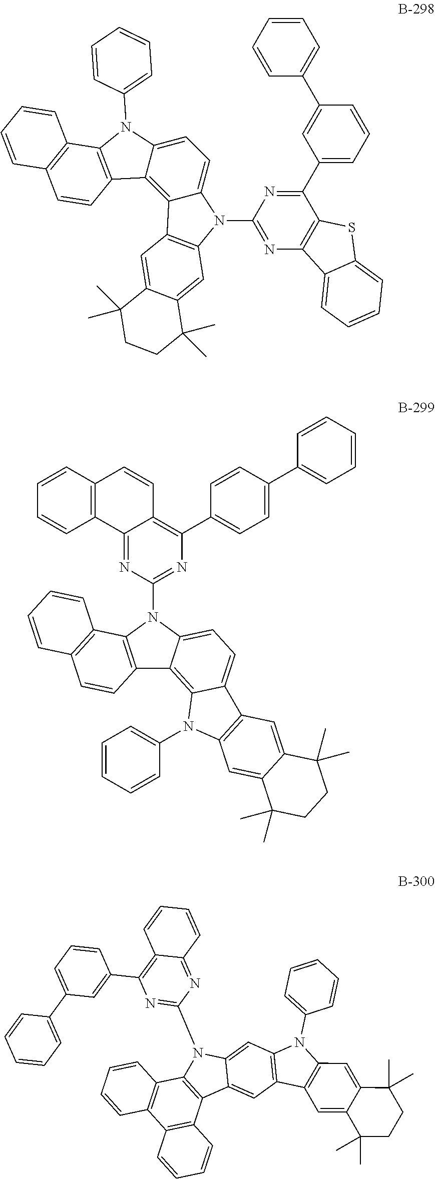 [SMILES: CC1(C)CCC(C)(C)c2cc3c(cc21)c1c2c4ccc5ccccc5c4n(-c4ccccc4)c2ccc1n3-c1nc(-c2cccc(-c3ccccc3)c2)c2sc3ccccc3c2n1 ; CC1(C)CCC(C)(C)c2cc3c(cc21)c1ccc2c(c4ccc5ccccc5c4n2-c2nc(-c4ccc(-c5ccccc5)cc4)c4ccc5ccccc5c4n2)c1n3-c1ccccc1 ; CC1(C)CCC(C)(C)c2cc3c(cc21)c1cc2c4c5ccccc5c5ccccc5c4n(-c4nc(-c5cccc(-c6ccccc6)c5)c5ccccc5n4)c2cc1n3-c1ccccc1]

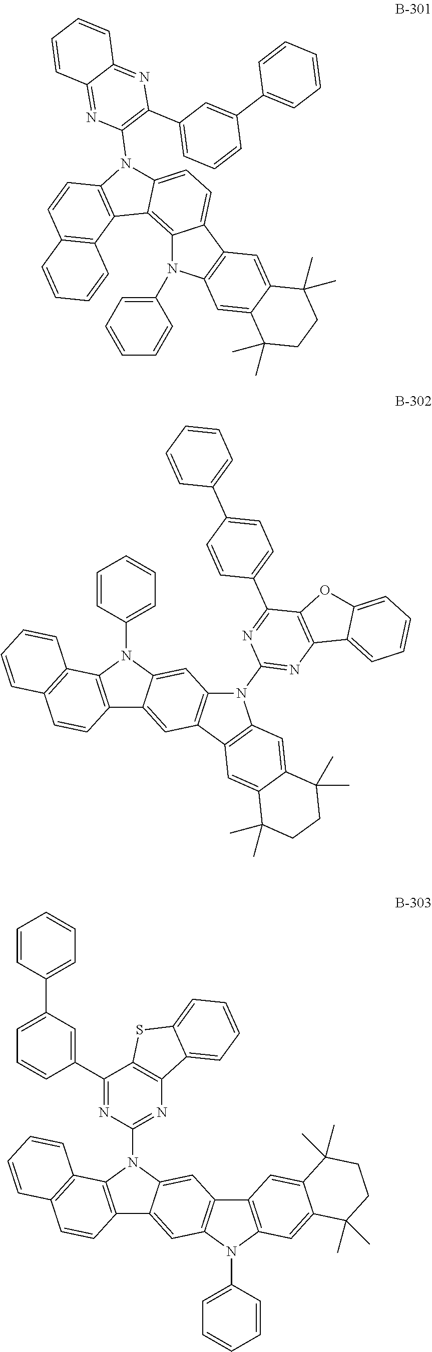 [SMILES: CC1(C)CCC(C)(C)c2cc3c(cc21)c1ccc2c(c4c5ccccc5ccc4n2-c2nc4ccccc4nc2-c2cccc(-c4ccccc4)c2)c1n3-c1ccccc1 ; CC1(C)CCC(C)(C)c2cc3c(cc21)c1cc2c4ccc5ccccc5c4n(-c4ccccc4)c2cc1n3-c1nc(-c2ccc(-c3ccccc3)cc2)c2oc3ccccc3c2n1 ; CC1(C)CCC(C)(C)c2cc3c(cc21)c1cc2c(cc1n3-c1ccccc1)c1ccc3ccccc3c1n2-c1nc(-c2cccc(-c3ccccc3)c2)c2sc3ccccc3c2n1]

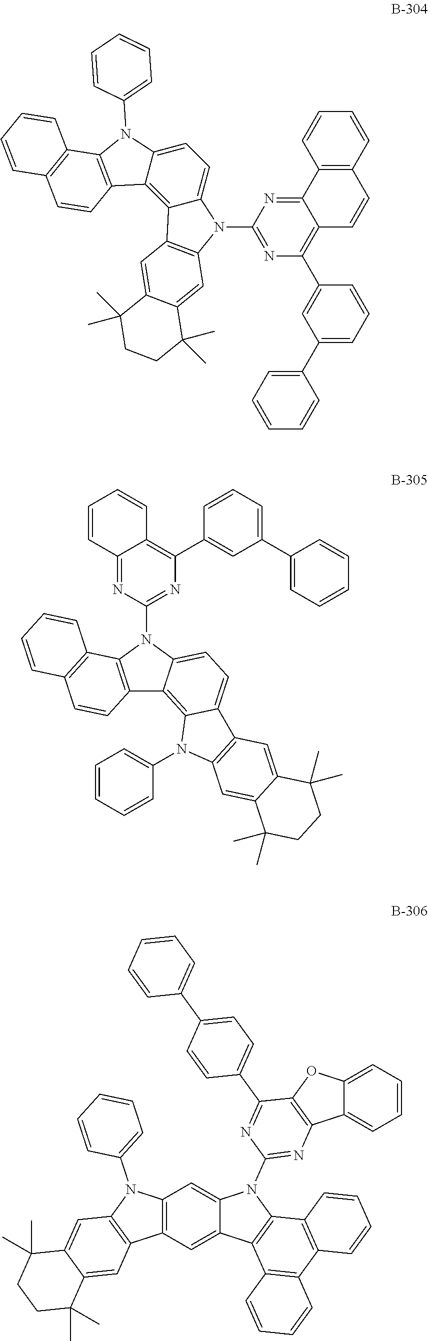 [SMILES: CC1(C)CCC(C)(C)c2cc3c(cc21)c1c2c4ccc5ccccc5c4n(-c4ccccc4)c2ccc1n3-c1nc(-c2cccc(-c3ccccc3)c2)c2ccc3ccccc3c2n1 ; CC1(C)CCC(C)(C)c2cc3c(cc21)c1ccc2c(c4ccc5ccccc5c4n2-c2nc(-c4cccc(-c5ccccc5)c4)c4ccccc4n2)c1n3-c1ccccc1 ; CC1(C)CCC(C)(C)c2cc3c(cc21)c1cc2c4c5ccccc5c5ccccc5c4n(-c4nc(-c5ccc(-c6ccccc6)cc5)c5oc6ccccc6c5n4)c2cc1n3-c1ccccc1]

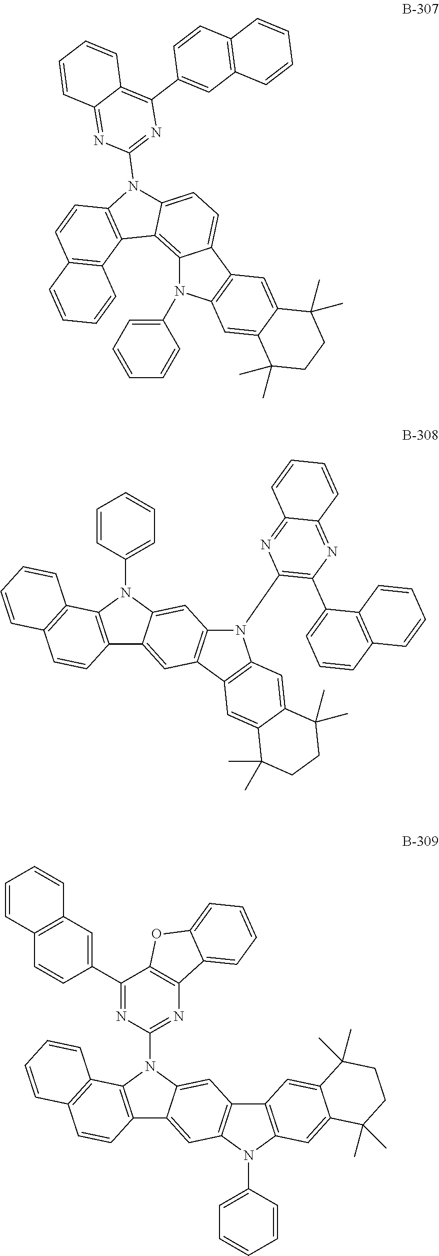 [SMILES: CC1(C)CCC(C)(C)c2cc3c(cc21)c1ccc2c(c4c5ccccc5ccc4n2-c2nc(-c4ccc5ccccc5c4)c4ccccc4n2)c1n3-c1ccccc1 ; CC1(C)CCC(C)(C)c2cc3c(cc21)c1cc2c4ccc5ccccc5c4n(-c4ccccc4)c2cc1n3-c1nc2ccccc2nc1-c1cccc2ccccc12 ; CC1(C)CCC(C)(C)c2cc3c(cc21)c1cc2c(cc1n3-c1ccccc1)c1ccc3ccccc3c1n2-c1nc(-c2ccc3ccccc3c2)c2oc3ccccc3c2n1]

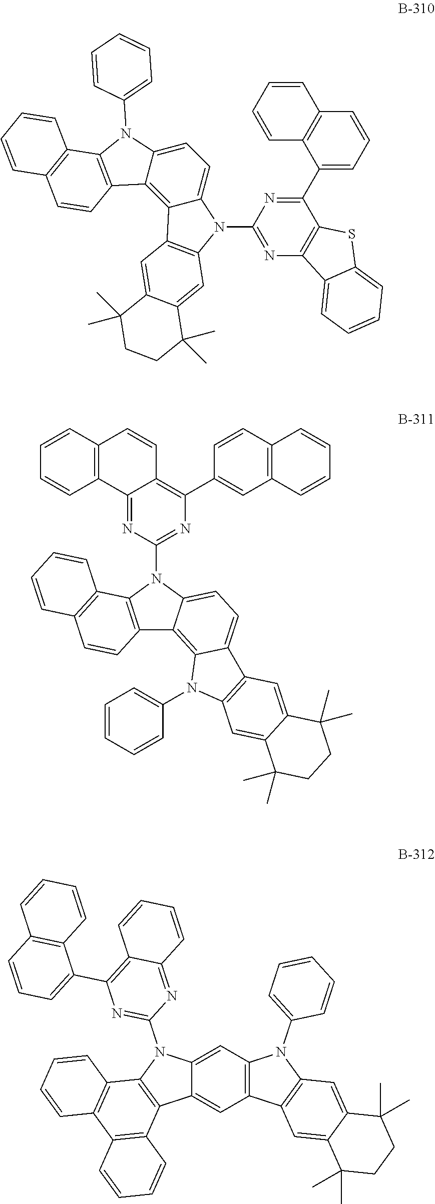 [SMILES: CC1(C)CCC(C)(C)c2cc3c(cc21)c1c2c4ccc5ccccc5c4n(-c4ccccc4)c2ccc1n3-c1nc(-c2cccc3ccccc23)c2sc3ccccc3c2n1 ; CC1(C)CCC(C)(C)c2cc3c(cc21)c1ccc2c(c4ccc5ccccc5c4n2-c2nc(-c4ccc5ccccc5c4)c4ccc5ccccc5c4n2)c1n3-c1ccccc1 ; CC1(C)CCC(C)(C)c2cc3c(cc21)c1cc2c4c5ccccc5c5ccccc5c4n(-c4nc(-c5cccc6ccccc56)c5ccccc5n4)c2cc1n3-c1ccccc1]

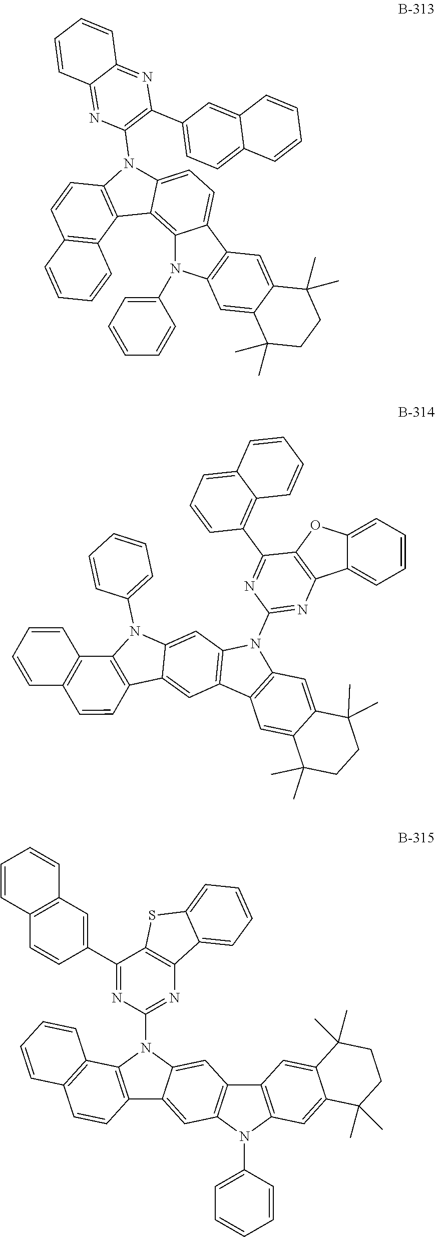 [SMILES: CC1(C)CCC(C)(C)c2cc3c(cc21)c1ccc2c(c4c5ccccc5ccc4n2-c2nc4ccccc4nc2-c2ccc4ccccc4c2)c1n3-c1ccccc1 ; CC1(C)CCC(C)(C)c2cc3c(cc21)c1cc2c4ccc5ccccc5c4n(-c4ccccc4)c2cc1n3-c1nc(-c2cccc3ccccc23)c2oc3ccccc3c2n1 ; CC1(C)CCC(C)(C)c2cc3c(cc21)c1cc2c(cc1n3-c1ccccc1)c1ccc3ccccc3c1n2-c1nc(-c2ccc3ccccc3c2)c2sc3ccccc3c2n1]

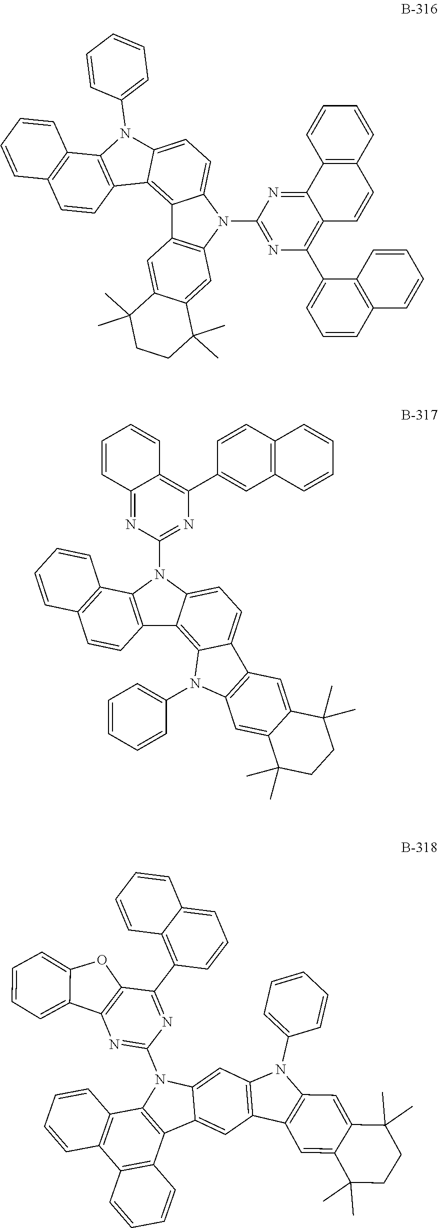 [SMILES: CC1(C)CCC(C)(C)c2cc3c(cc21)c1c2c4ccc5ccccc5c4n(-c4ccccc4)c2ccc1n3-c1nc(-c2cccc3ccccc23)c2ccc3ccccc3c2n1 ; CC1(C)CCC(C)(C)c2cc3c(cc21)c1ccc2c(c4ccc5ccccc5c4n2-c2nc(-c4ccc5ccccc5c4)c4ccccc4n2)c1n3-c1ccccc1 ; CC1(C)CCC(C)(C)c2cc3c(cc21)c1cc2c4c5ccccc5c5ccccc5c4n(-c4nc(-c5cccc6ccccc56)c5oc6ccccc6c5n4)c2cc1n3-c1ccccc1]

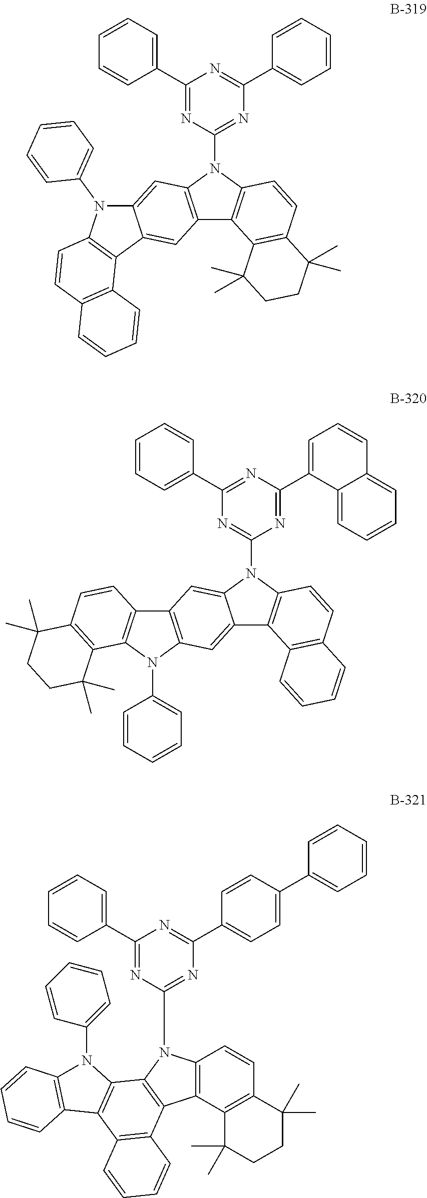

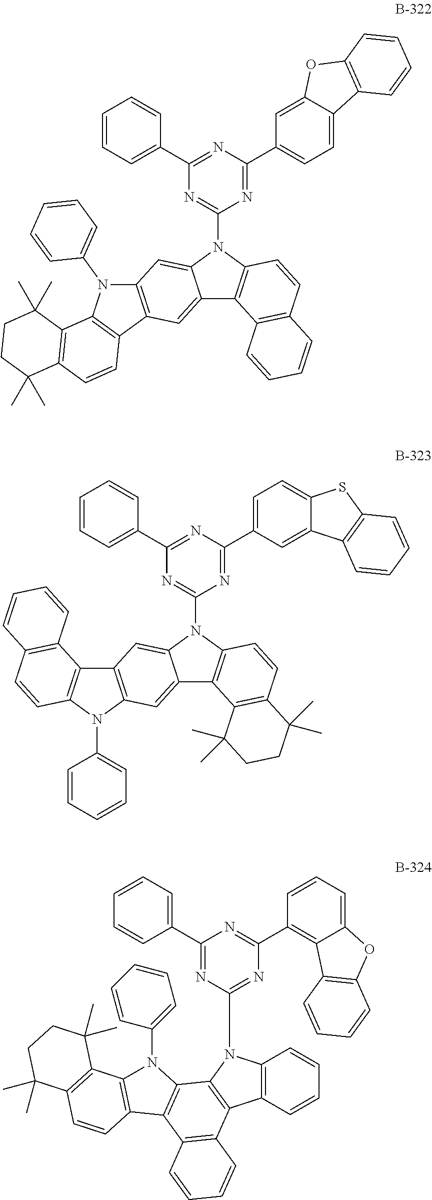 [SMILES: CC1(C)CCC(C)(C)c2c1ccc1c3cc4c5c6ccccc6ccc5n(-c5nc(-c6ccccc6)nc(-c6ccc7c(c6)oc6ccccc67)n5)c4cc3n(-c3ccccc3)c21 ; CC1(C)CCC(C)(C)c2c1ccc1c2c2cc3c(cc2n1-c1nc(-c2ccccc2)nc(-c2ccc4sc5ccccc5c4c2)n1)c1c2ccccc2ccc1n3-c1ccccc1 ; CC1(C)CCC(C)(C)c2c1ccc1c3c4ccccc4c4c5ccccc5n(-c5nc(-c6ccccc6)nc(-c6cccc7oc8ccccc8c67)n5)c4c3n(-c3ccccc3)c21]

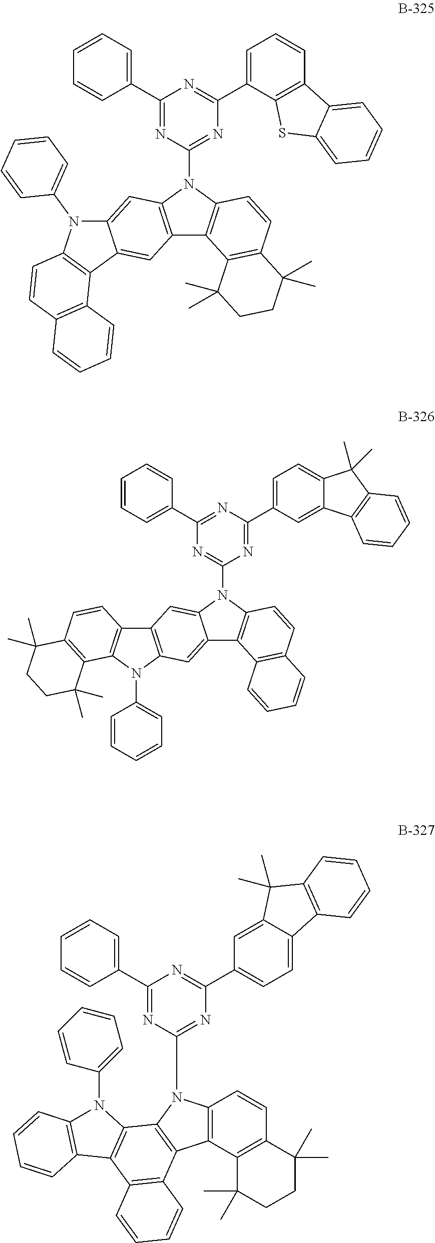 [SMILES: CC1(C)CCC(C)(C)c2c1ccc1c2c2cc3c4c5ccccc5ccc4n(-c4ccccc4)c3cc2n1-c1nc(-c2ccccc2)nc(-c2cccc3c2sc2ccccc23)n1 ; CC1(C)CCC(C)(C)c2c1ccc1c3cc4c(cc3n(-c3ccccc3)c21)c1c2ccccc2ccc1n4-c1nc(-c2ccccc2)nc(-c2ccc3c(c2)-c2ccccc2C3(C)C)n1 ; CC1(C)CCC(C)(C)c2c1ccc1c2c2c3ccccc3c3c4ccccc4n(-c4ccccc4)c3c2n1-c1nc(-c2ccccc2)nc(-c2ccc3c(c2)C(C)(C)c2ccccc2-3)n1]

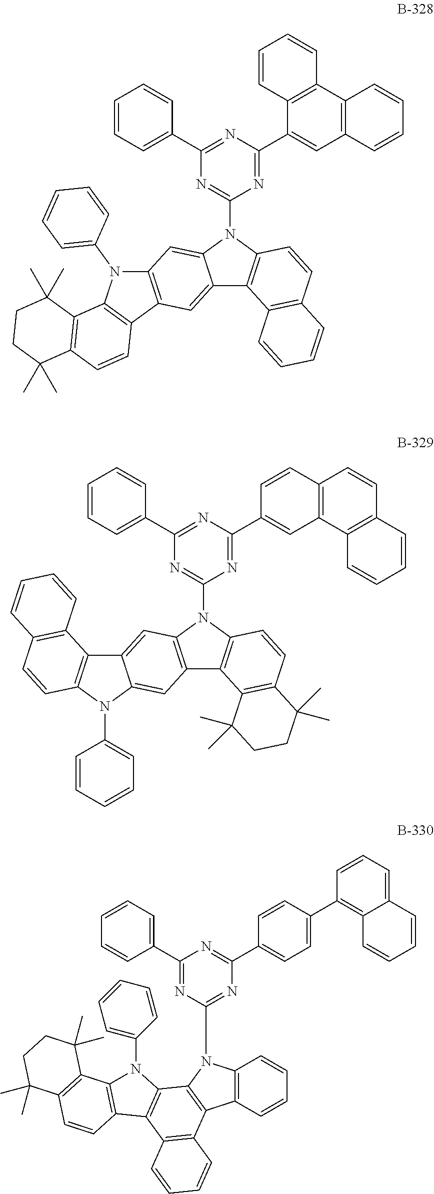 [SMILES: CC1(C)CCC(C)(C)c2c1ccc1c3cc4c5c6ccccc6ccc5n(-c5nc(-c6ccccc6)nc(-c6cc7ccccc7c7ccccc67)n5)c4cc3n(-c3ccccc3)c21 ; CC1(C)CCC(C)(C)c2c1ccc1c2c2cc3c(cc2n1-c1nc(-c2ccccc2)nc(-c2ccc4ccc5ccccc5c4c2)n1)c1c2ccccc2ccc1n3-c1ccccc1 ; CC1(C)CCC(C)(C)c2c1ccc1c3c4ccccc4c4c5ccccc5n(-c5nc(-c6ccccc6)nc(-c6ccc(-c7cccc8ccccc78)cc6)n5)c4c3n(-c3ccccc3)c21]

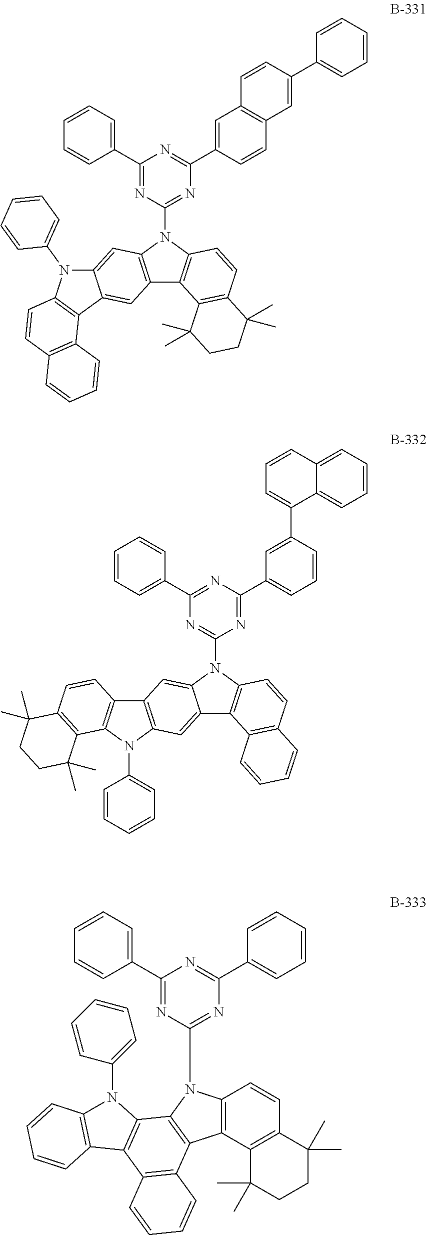 [SMILES: CC1(C)CCC(C)(C)c2c1ccc1c2c2cc3c4c5ccccc5ccc4n(-c4ccccc4)c3cc2n1-c1nc(-c2ccccc2)nc(-c2ccc3cc(-c4ccccc4)ccc3c2)n1 ; CC1(C)CCC(C)(C)c2c1ccc1c3cc4c(cc3n(-c3ccccc3)c21)c1c2ccccc2ccc1n4-c1nc(-c2ccccc2)nc(-c2cccc(-c3cccc4ccccc34)c2)n1 ; CC1(C)CCC(C)(C)c2c1ccc1c2c2c3ccccc3c3c4ccccc4n(-c4ccccc4)c3c2n1-c1nc(-c2ccccc2)nc(-c2ccccc2)n1]

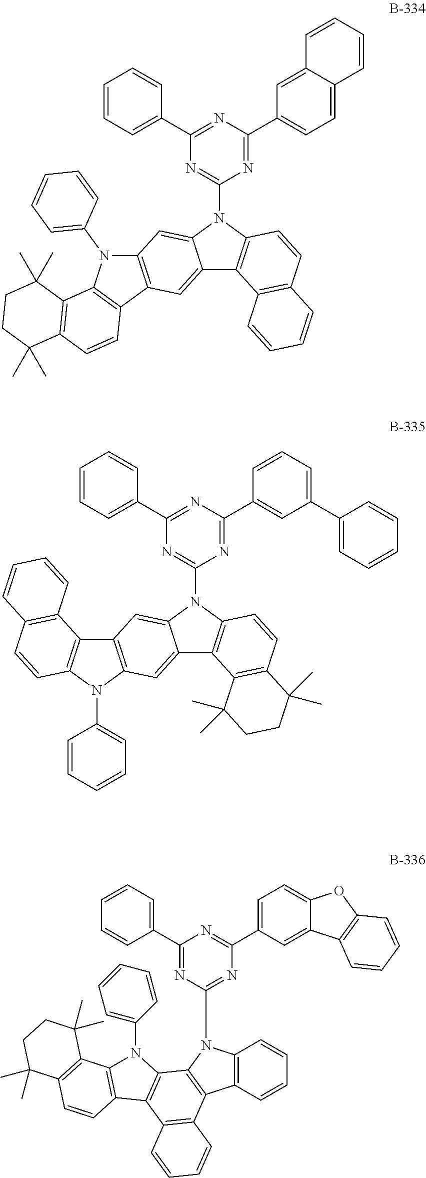 [SMILES: CC1(C)CCC(C)(C)c2c1ccc1c3cc4c5c6ccccc6ccc5n(-c5nc(-c6ccccc6)nc(-c6ccc7ccccc7c6)n5)c4cc3n(-c3ccccc3)c21 ; CC1(C)CCC(C)(C)c2c1ccc1c2c2cc3c(cc2n1-c1nc(-c2ccccc2)nc(-c2cccc(-c4ccccc4)c2)n1)c1c2ccccc2ccc1n3-c1ccccc1 ; CC1(C)CCC(C)(C)c2c1ccc1c3c4ccccc4c4c5ccccc5n(-c5nc(-c6ccccc6)nc(-c6ccc7oc8ccccc8c7c6)n5)c4c3n(-c3ccccc3)c21]

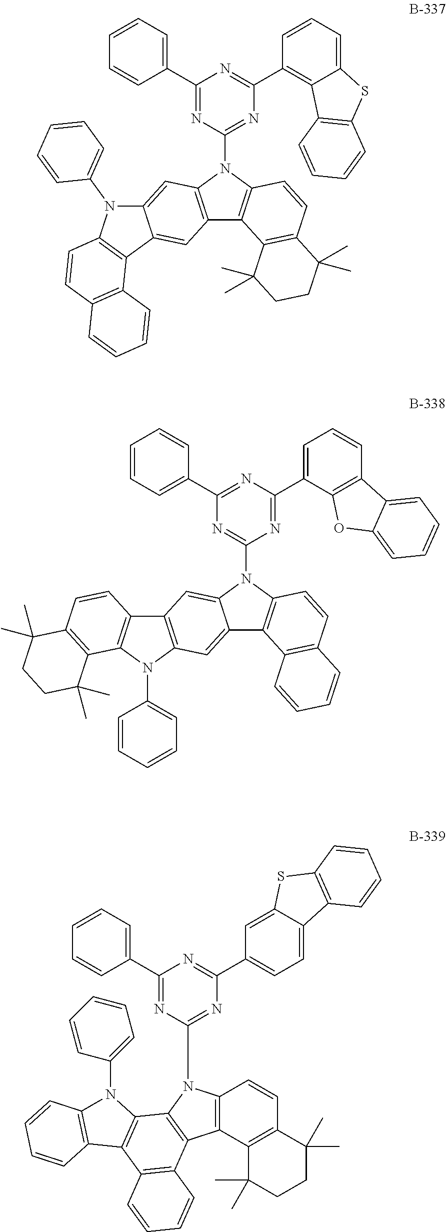 [SMILES: CC1(C)CCC(C)(C)c2c1ccc1c2c2cc3c4c5ccccc5ccc4n(-c4ccccc4)c3cc2n1-c1nc(-c2ccccc2)nc(-c2cccc3sc4ccccc4c23)n1 ; CC1(C)CCC(C)(C)c2c1ccc1c3cc4c(cc3n(-c3ccccc3)c21)c1c2ccccc2ccc1n4-c1nc(-c2ccccc2)nc(-c2cccc3c2oc2ccccc23)n1 ; CC1(C)CCC(C)(C)c2c1ccc1c2c2c3ccccc3c3c4ccccc4n(-c4ccccc4)c3c2n1-c1nc(-c2ccccc2)nc(-c2ccc3c(c2)sc2ccccc23)n1]

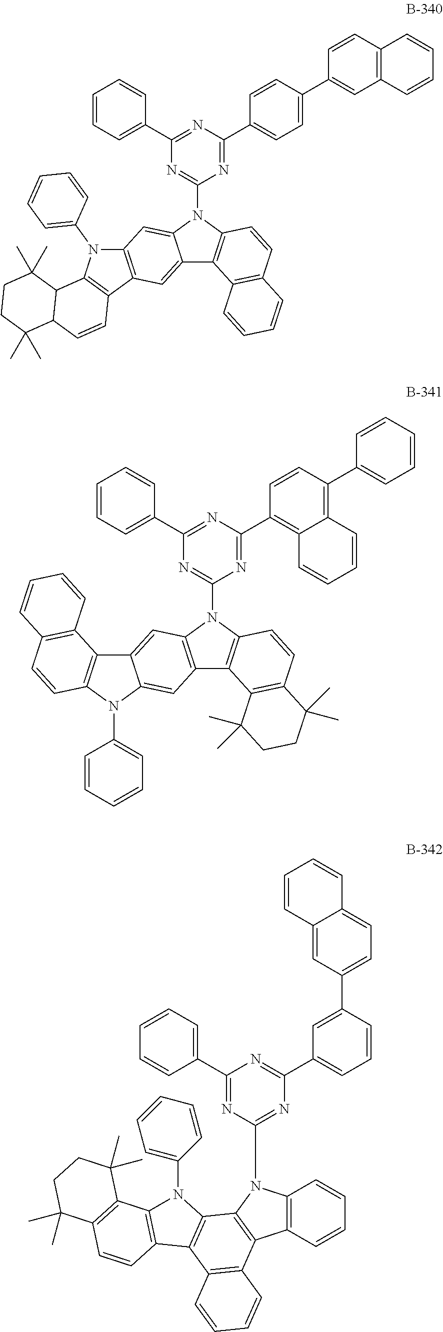 [SMILES: CC1(C)CCC(C)(C)C2c3c(c4cc5c6c7ccccc7ccc6n(-c6nc(-c7ccccc7)nc(-c7ccc(-c8ccc9ccccc9c8)cc7)n6)c5cc4n3-c3ccccc3)C=CC21 ; CC1(C)CCC(C)(C)c2c1ccc1c2c2cc3c(cc2n1-c1nc(-c2ccccc2)nc(-c2ccc(-c4ccccc4)c4ccccc24)n1)c1c2ccccc2ccc1n3-c1ccccc1 ; CC1(C)CCC(C)(C)c2c1ccc1c3c4ccccc4c4c5ccccc5n(-c5nc(-c6ccccc6)nc(-c6cccc(-c7ccc8ccccc8c7)c6)n5)c4c3n(-c3ccccc3)c21]

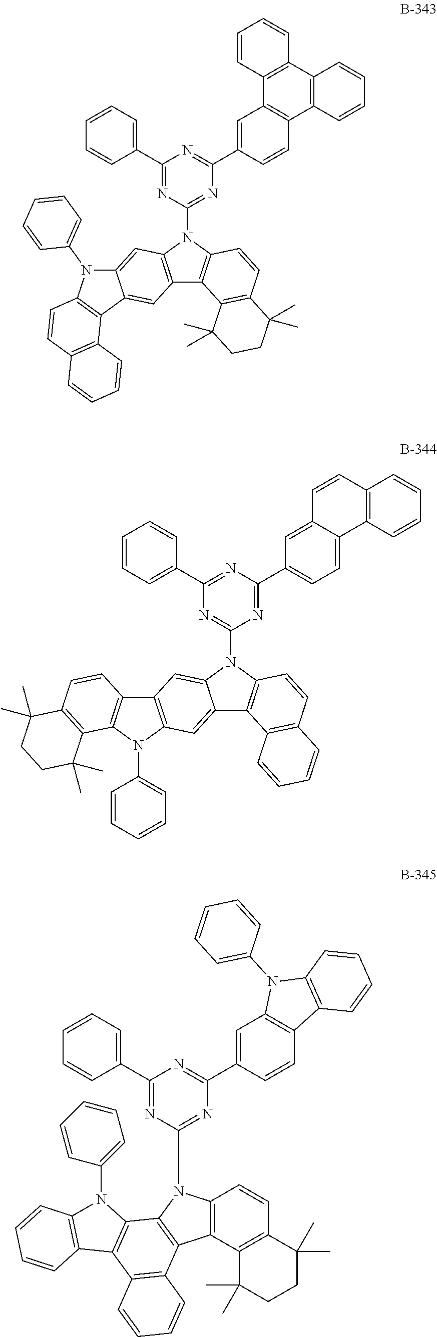 [SMILES: CC1(C)CCC(C)(C)c2c1ccc1c2c2cc3c4c5ccccc5ccc4n(-c4ccccc4)c3cc2n1-c1nc(-c2ccccc2)nc(-c2ccc3c4ccccc4c4ccccc4c3c2)n1 ; CC1(C)CCC(C)(C)c2c1ccc1c3cc4c(cc3n(-c3ccccc3)c21)c1c2ccccc2ccc1n4-c1nc(-c2ccccc2)nc(-c2ccc3c(ccc4ccccc43)c2)n1 ; CC1(C)CCC(C)(C)c2c1ccc1c2c2c3ccccc3c3c4ccccc4n(-c4ccccc4)c3c2n1-c1nc(-c2ccccc2)nc(-c2ccc3c4ccccc4n(-c4ccccc4)c3c2)n1]

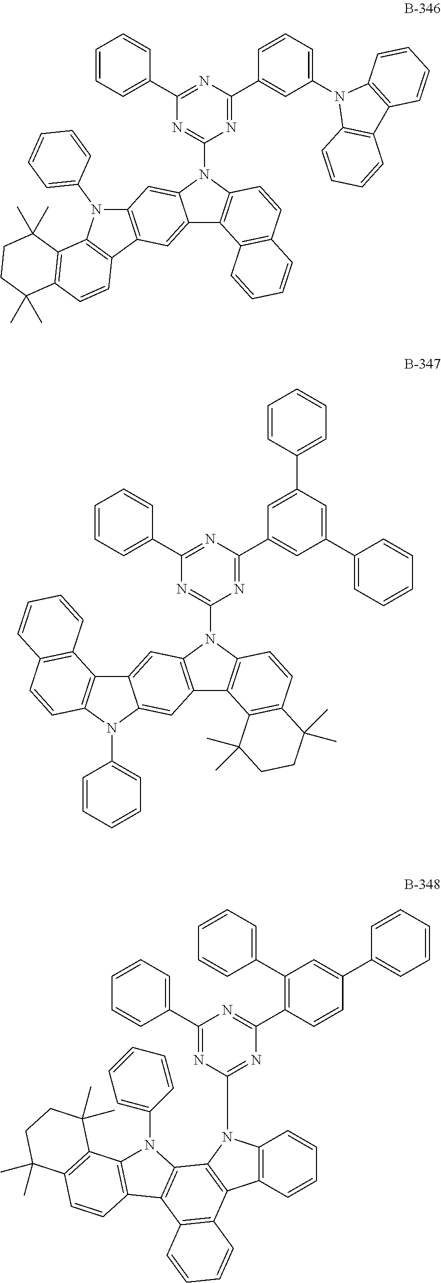 [SMILES: CC1(C)CCC(C)(C)c2c1ccc1c3cc4c5c6ccccc6ccc5n(-c5nc(-c6ccccc6)nc(-c6cccc(-n7c8ccccc8c8ccccc87)c6)n5)c4cc3n(-c3ccccc3)c21 ; CC1(C)CCC(C)(C)c2c1ccc1c2c2cc3c(cc2n1-c1nc(-c2ccccc2)nc(-c2cc(-c4ccccc4)cc(-c4ccccc4)c2)n1)c1c2ccccc2ccc1n3-c1ccccc1 ; CC1(C)CCC(C)(C)c2c1ccc1c3c4ccccc4c4c5ccccc5n(-c5nc(-c6ccccc6)nc(-c6ccc(-c7ccccc7)cc6-c6ccccc6)n5)c4c3n(-c3ccccc3)c21]

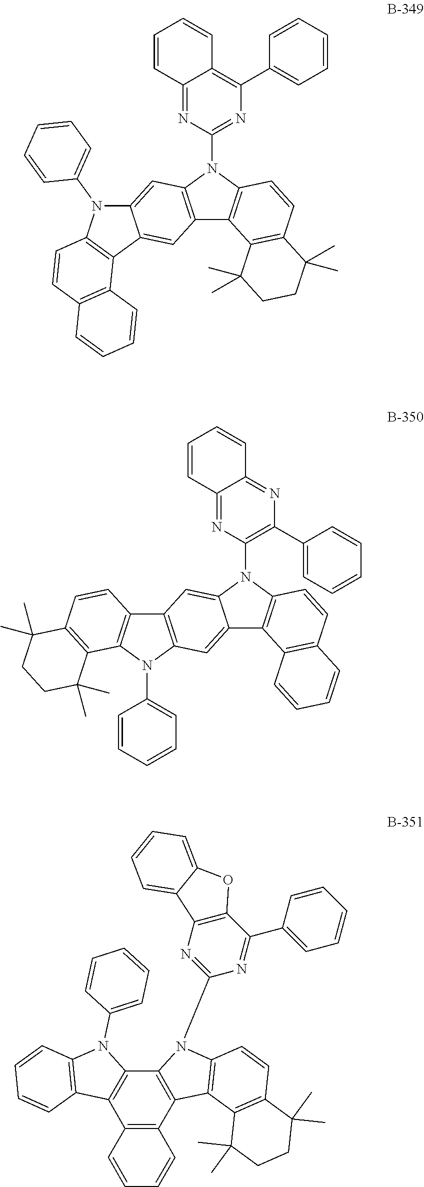

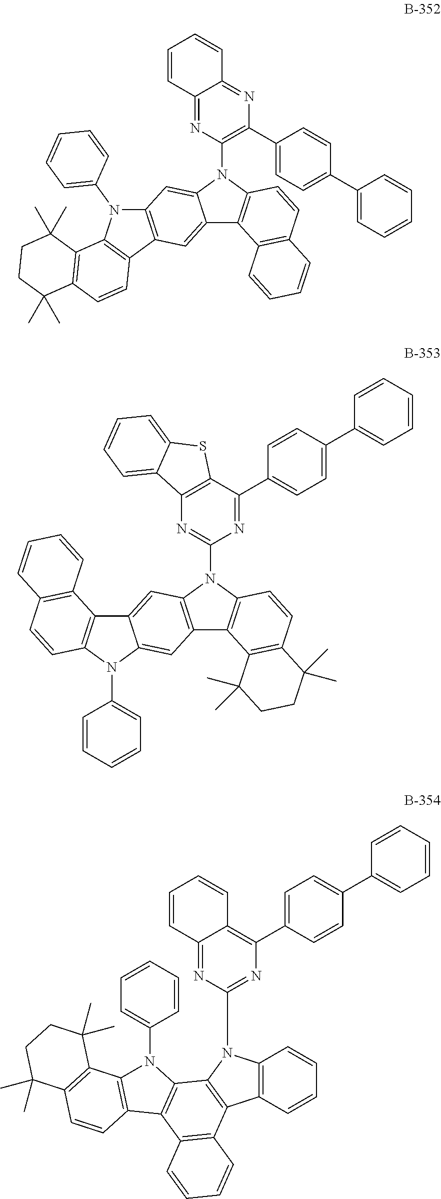 [SMILES: CC1(C)CCC(C)(C)c2c1ccc1c3cc4c5c6ccccc6ccc5n(-c5nc6ccccc6nc5-c5ccc(-c6ccccc6)cc5)c4cc3n(-c3ccccc3)c21 ; CC1(C)CCC(C)(C)c2c1ccc1c2c2cc3c(cc2n1-c1nc(-c2ccc(-c4ccccc4)cc2)c2sc4ccccc4c2n1)c1c2ccccc2ccc1n3-c1ccccc1 ; CC1(C)CCC(C)(C)c2c1ccc1c3c4ccccc4c4c5ccccc5n(-c5nc(-c6ccc(-c7ccccc7)cc6)c6ccccc6n5)c4c3n(-c3ccccc3)c21]

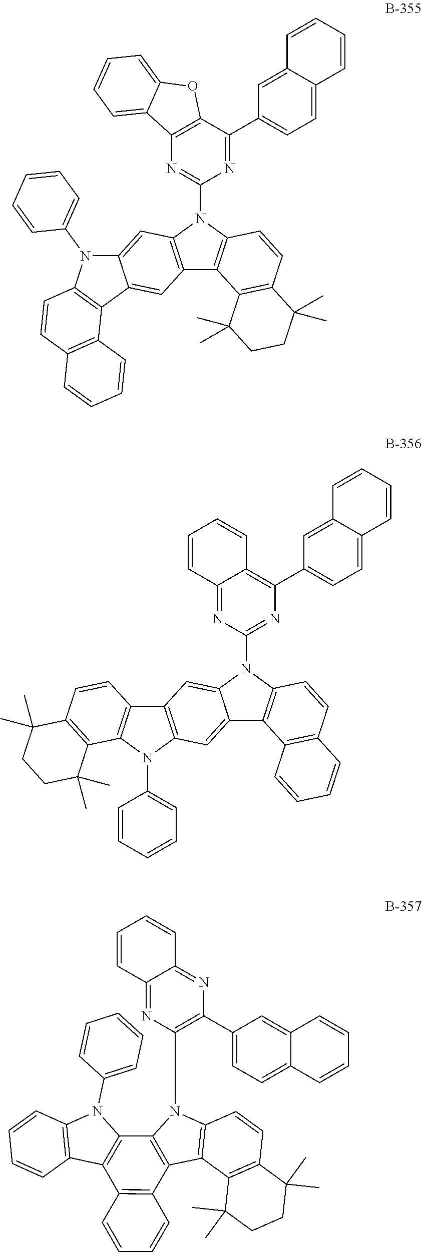 [SMILES: CC1(C)CCC(C)(C)c2c1ccc1c2c2cc3c4c5ccccc5ccc4n(-c4ccccc4)c3cc2n1-c1nc(-c2ccc3ccccc3c2)c2oc3ccccc3c2n1 ; CC1(C)CCC(C)(C)c2c1ccc1c3cc4c(cc3n(-c3ccccc3)c21)c1c2ccccc2ccc1n4-c1nc(-c2ccc3ccccc3c2)c2ccccc2n1 ; CC1(C)CCC(C)(C)c2c1ccc1c2c2c3ccccc3c3c4ccccc4n(-c4ccccc4)c3c2n1-c1nc2ccccc2nc1-c1ccc2ccccc2c1]

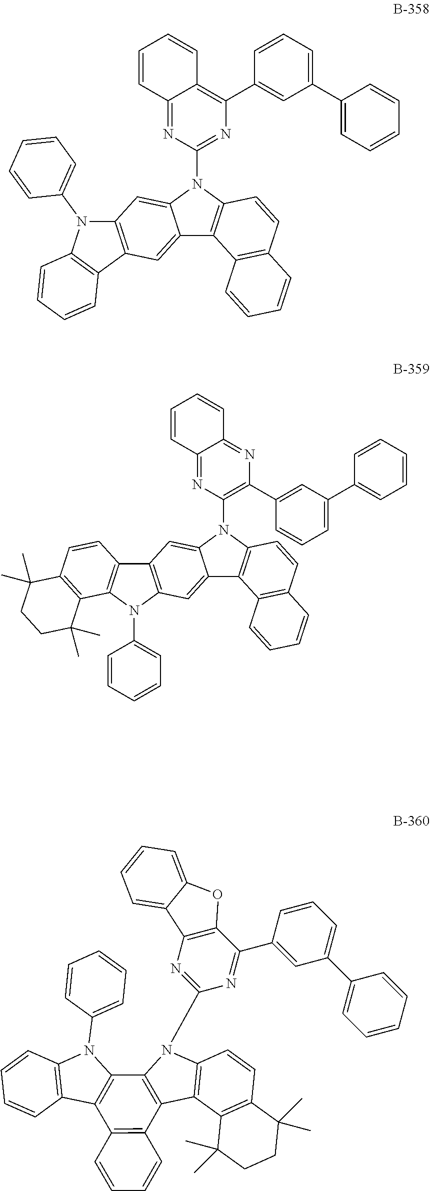 [SMILES: c1ccc(-c2cccc(-c3nc(-n4c5cc6c(cc5c5c7ccccc7ccc54)c4ccccc4n6-c4ccccc4)nc4ccccc34)c2)cc1 ; CC1(C)CCC(C)(C)c2c1ccc1c3cc4c(cc3n(-c3ccccc3)c21)c1c2ccccc2ccc1n4-c1nc2ccccc2nc1-c1cccc(-c2ccccc2)c1 ; CC1(C)CCC(C)(C)c2c1ccc1c2c2c3ccccc3c3c4ccccc4n(-c4ccccc4)c3c2n1-c1nc(-c2cccc(-c3ccccc3)c2)c2oc3ccccc3c2n1]

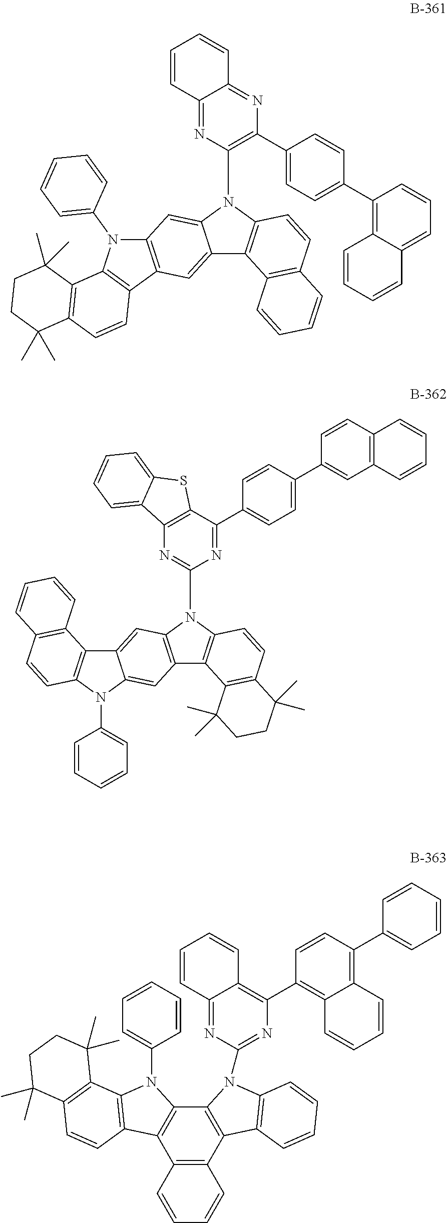 [SMILES: CC1(C)CCC(C)(C)c2c1ccc1c3cc4c5c6ccccc6ccc5n(-c5nc6ccccc6nc5-c5ccc(-c6cccc7ccccc67)cc5)c4cc3n(-c3ccccc3)c21 ; CC1(C)CCC(C)(C)c2c1ccc1c2c2cc3c(cc2n1-c1nc(-c2ccc(-c4ccc5ccccc5c4)cc2)c2sc4ccccc4c2n1)c1c2ccccc2ccc1n3-c1ccccc1 ; CC1(C)CCC(C)(C)c2c1ccc1c3c4ccccc4c4c5ccccc5n(-c5nc(-c6ccc(-c7ccccc7)c7ccccc67)c6ccccc6n5)c4c3n(-c3ccccc3)c21]

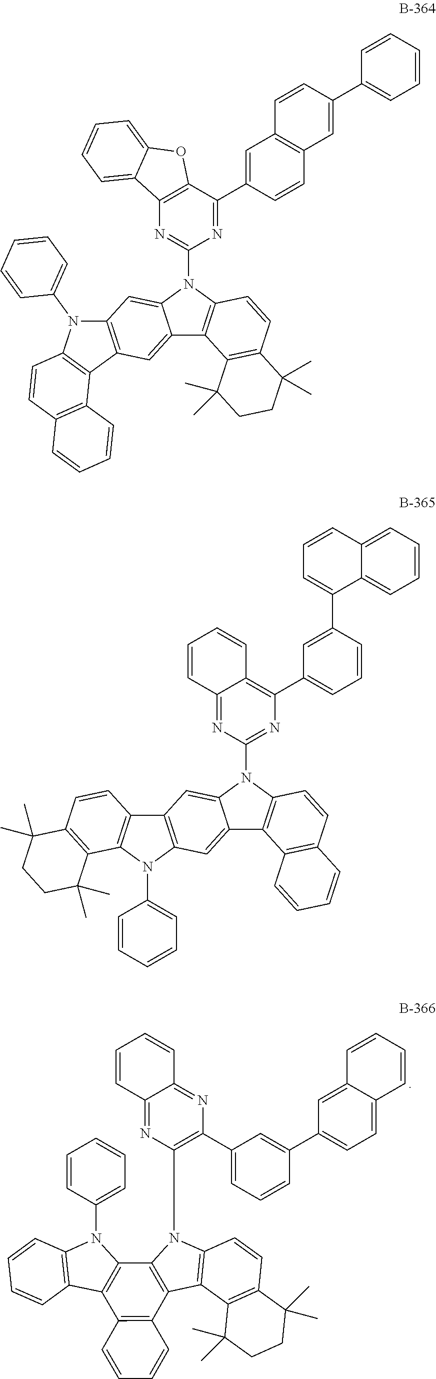 [SMILES: CC1(C)CCC(C)(C)c2c1ccc1c2c2cc3c4c5ccccc5ccc4n(-c4ccccc4)c3cc2n1-c1nc(-c2ccc3cc(-c4ccccc4)ccc3c2)c2oc3ccccc3c2n1 ; CC1(C)CCC(C)(C)c2c1ccc1c3cc4c(cc3n(-c3ccccc3)c21)c1c2ccccc2ccc1n4-c1nc(-c2cccc(-c3cccc4ccccc34)c2)c2ccccc2n1 ; CC1(C)CCC(C)(C)c2c1ccc1c2c2c3ccccc3c3c4ccccc4n(-c4ccccc4)c3c2n1-c1nc2ccccc2nc1-c1cccc(-c2ccc3ccccc3c2)c1]

In a second aspect, the present application provides an organic electroluminescent device comprising an anode, a cathode, and a functional layer disposed between the anode and the cathode. The functional layer comprises the nitrogen-containing compound described in the first aspect of the present application.

The nitrogen-containing compound provided in the present application may be used to form at least one organic film layer in the functional layer so as to improve properties of the organic electroluminescent device such as luminescence efficiency and service life.

Alternatively, the functional layer includes an organic light emitting layer comprising the nitrogen-containing compound. The organic light emitting layer may be composed of the nitrogen-containing compound provided in the present application, or may be composed of the nitrogen-containing compound provided in the present application together with other materials.

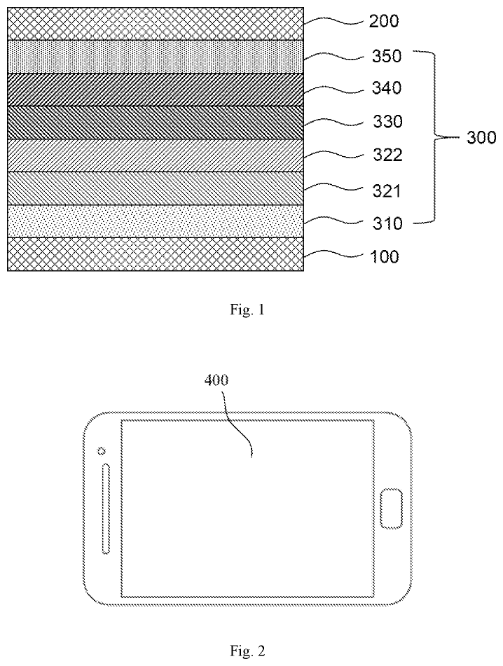

According to a specific embodiment, the organic electroluminescent device is as shown in FIG. 1. The organic electroluminescent device may include an anode 100, a hole injection layer 310, a first hole transport layer 321, a second hole transport layer (hole auxiliary layer) 322, an organic light emitting layer 330, an electron transport layer 340, an electron injection layer 350, and a cathode 200 that are stacked in sequence.

In the present application, the anode 100 comprises an anode material, which is preferably a large work function material contributing to injection of holes into the functional layer. Specific examples of the anode material include, but are not limited to: metals such as nickel, platinum, vanadium, chromium, copper, zinc, gold, and alloys thereof; metal oxides such as zinc oxide, indium oxide, indium tin oxide (ITO), and indium zinc oxide (IZO); combinations of metals and oxides, such as ZnO:Al or SnO₂:Sb; and conductive polymers such as poly(3-methylthiophene), poly[3,4-(ethylene-1,2-dioxy)thiophene] (PEDT), polypyrrole, and polyaniline. Preferably, a transparent electrode comprising indium tin oxide (ITO) is included as the anode.

In the present application, the hole transport layer may comprise one or more hole transport materials. The transport materials may be selected from carbazole polymers, carbazole-linked triarylamine compounds and other types of compounds, and may be specifically selected from the following compounds or any combinations thereof:

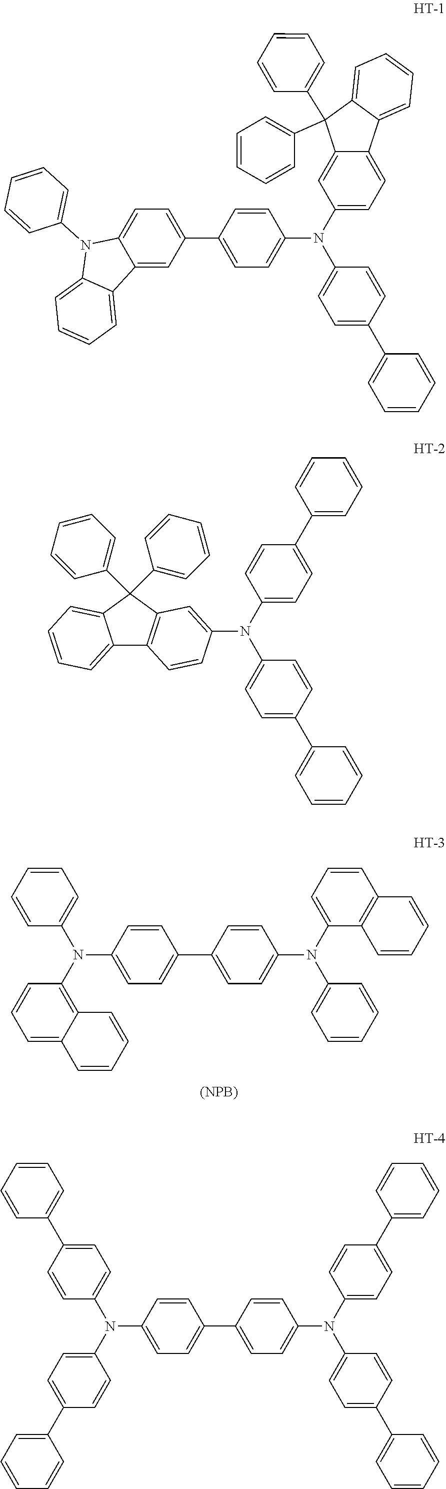 [SMILES: c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(-c2ccc(N(c3ccc(-c4ccccc4)cc3)c3ccc4c(c3)C(c3ccccc3)(c3ccccc3)c3ccccc3-4)cc2)cc1 ; c1ccc(N(c2ccc(-c3ccc(N(c4ccccc4)c4cccc5ccccc45)cc3)cc2)c2cccc3ccccc23)cc1 ; "(NPB)" ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1]

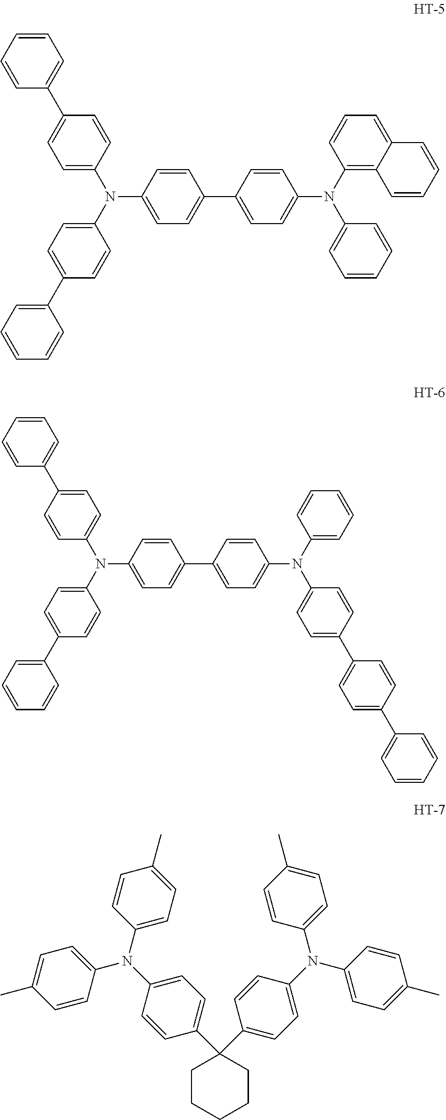

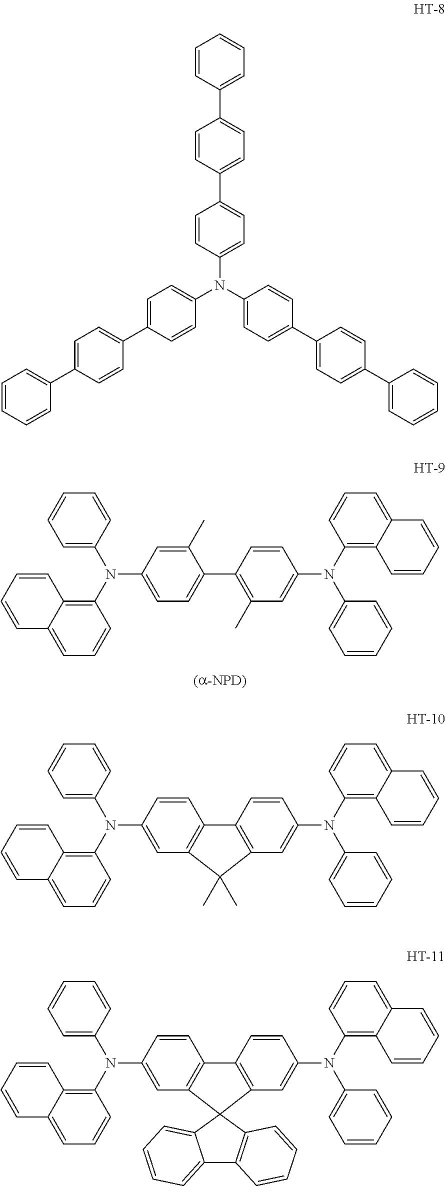 [SMILES: c1ccc(-c2ccc(-c3ccc(N(c4ccc(-c5ccc(-c6ccccc6)cc5)cc4)c4ccc(-c5ccc(-c6ccccc6)cc5)cc4)cc3)cc2)cc1 ; Cc1cc(N(c2ccccc2)c2cccc3ccccc23)ccc1-c1ccc(N(c2ccccc2)c2cccc3ccccc23)cc1C ; CC1(C)c2cc(N(c3ccccc3)c3cccc4ccccc34)ccc2-c2ccc(N(c3ccccc3)c3cccc4ccccc34)cc21 ; c1ccc(N(c2ccc3c(c2)C2(c4ccccc4-c4ccccc42)c2cc(N(c4ccccc4)c4cccc5ccccc45)ccc2-3)c2cccc3ccccc23)cc1]

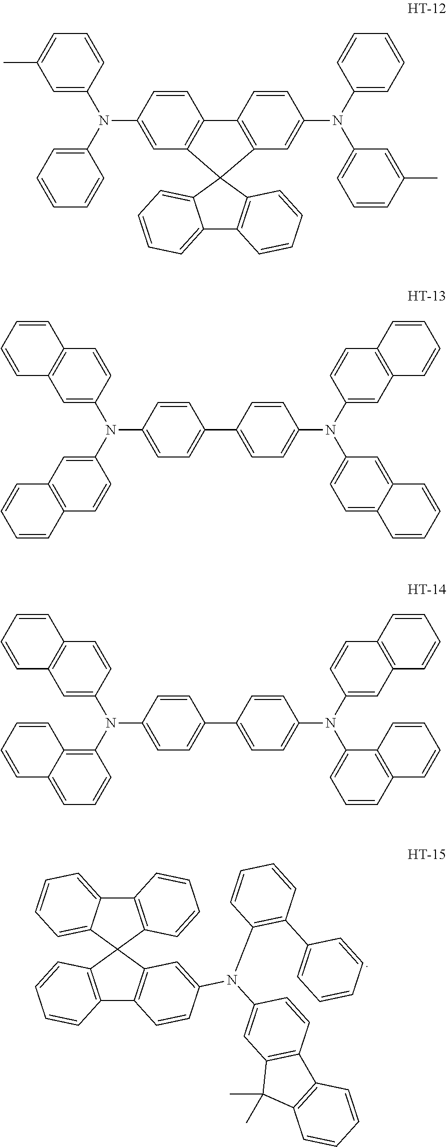

In one embodiment, the first hole transport layer 321 may be composed of HT-1 or NPB.

In one embodiment, the second hole transport layer 322 may be composed of HT-2 or HT-15.

Alternatively, a hole injection layer 310 is further provided between the anode 100 and the first hole transport layer 321 so as to enhance the ability to inject holes into the first hole transport layer 321. The hole injection layer 310 may be composed of a material selected from benzidine derivatives, starburst arylamine compounds, phthalocyanine derivatives, and other materials, which is not limited in the present application. The material of the hole injection layer 310 is, for example, selected from the following compounds or any combinations thereof:

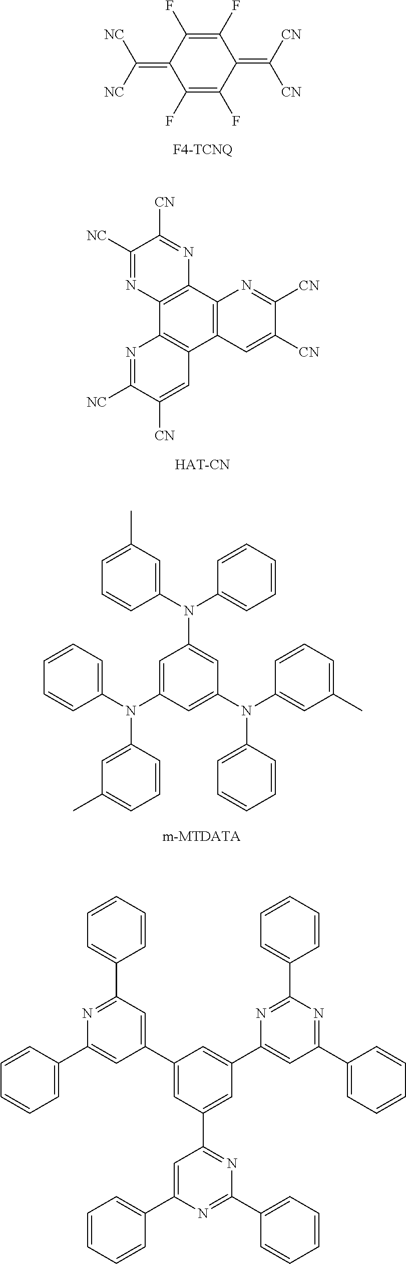 [SMILES: N#CC(C#N)=c1c(F)c(F)c(=C(C#N)C#N)c(F)c1F ; N#Cc1cc2c3cc(C#N)c(C#N)nc3c3nc(C#N)c(C#N)nc3c2nc1C#N ; Cc1cccc(N(c2ccccc2)c2cc(N(c3ccccc3)c3cccc(C)c3)cc(N(c3ccccc3)c3cccc(C)c3)c2)c1 ; c1ccc(-c2cc(-c3cc(-c4cc(-c5ccccc5)nc(-c5ccccc5)n4)cc(-c4cc(-c5ccccc5)nc(-c5ccccc5)n4)c3)cc(-c3ccccc3)n2)cc1]

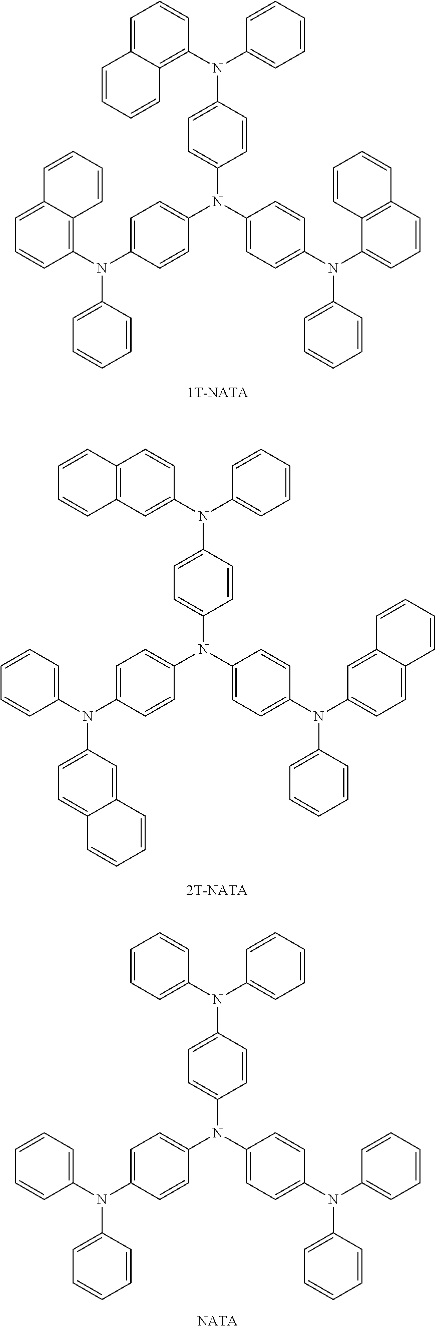

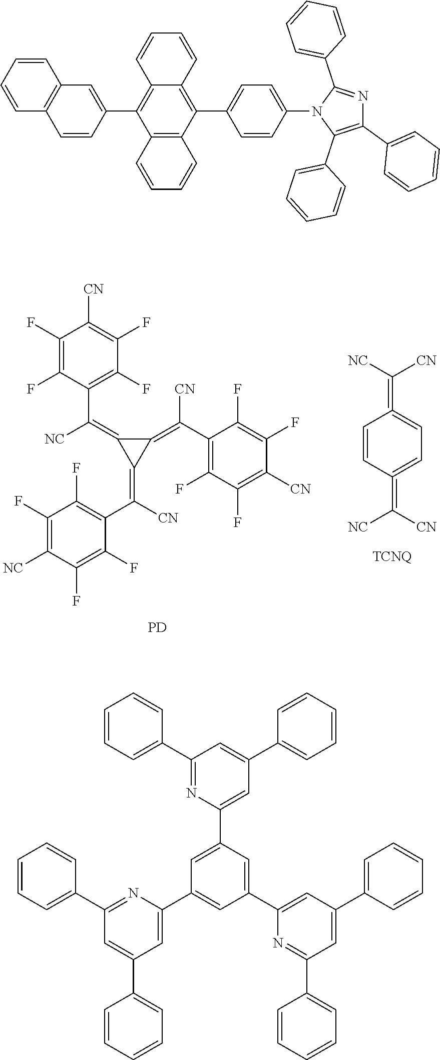

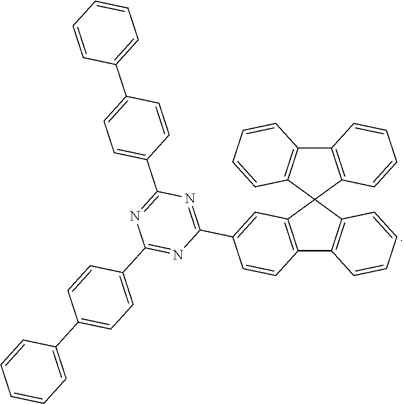 [SMILES: c1ccc(-c2ccc(-c3nc(-c4ccc(-c5ccccc5)cc4)nc(-c4ccc5c(c4)C4(c6ccccc6-c6ccccc64)c4ccccc4-5)n3)cc2)cc1]

In one embodiment, the hole injection layer 310 is composed of PD.

In the present application, the organic light emitting layer 330 may be composed of a single luminescent material, or may include a host material and a guest material. Alternatively, the organic light emitting layer 330 is composed of a host material and a guest material. Holes injected into the organic light emitting layer 330 and electrons injected into the organic light emitting layer 330 can be recombined in the organic light emitting layer 330 to form excitons. The excitons transmit energy to the host material, and the host material transmits the energy to the guest material, thereby enabling the guest material to emit light.

The host material of the organic light emitting layer 330 may comprise a metal chelating compound, a stilbene derivative, an aromatic amine derivative, a dibenzofuran derivative or other types of materials. Alternatively, the host material comprises the nitrogen-containing compound of the present application.

The guest material of the organic light emitting layer 330 may be a compound having a condensed aryl ring or a derivative thereof, a compound having a heteroaryl ring or a derivative thereof, an aromatic amine derivative, or other materials, which is not particularly limited in the present application. The guest material is also known as a doping material or a dopant, which can be categorized, according to its type of luminescence, as a fluorescent dopant or a phosphorescent dopant. Specific examples of the phosphorescent dopant include, but are not limited to:

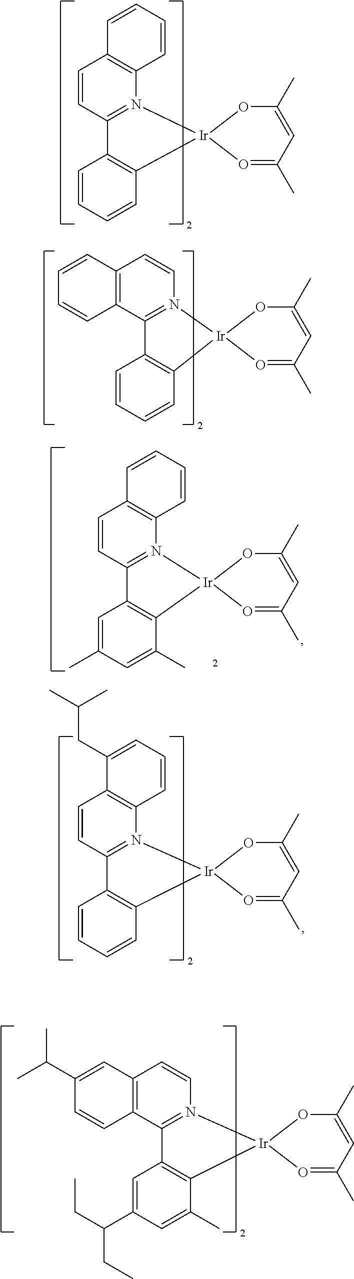

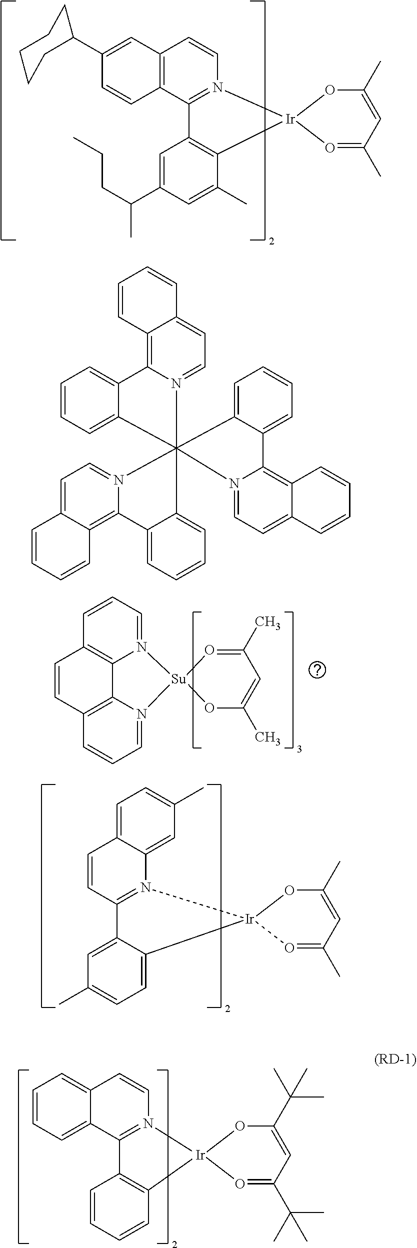

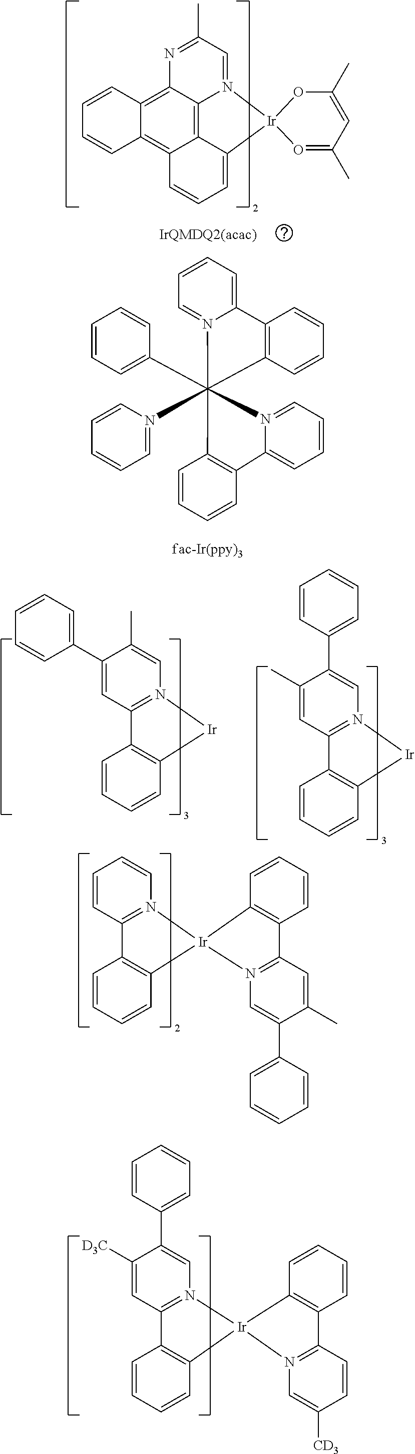

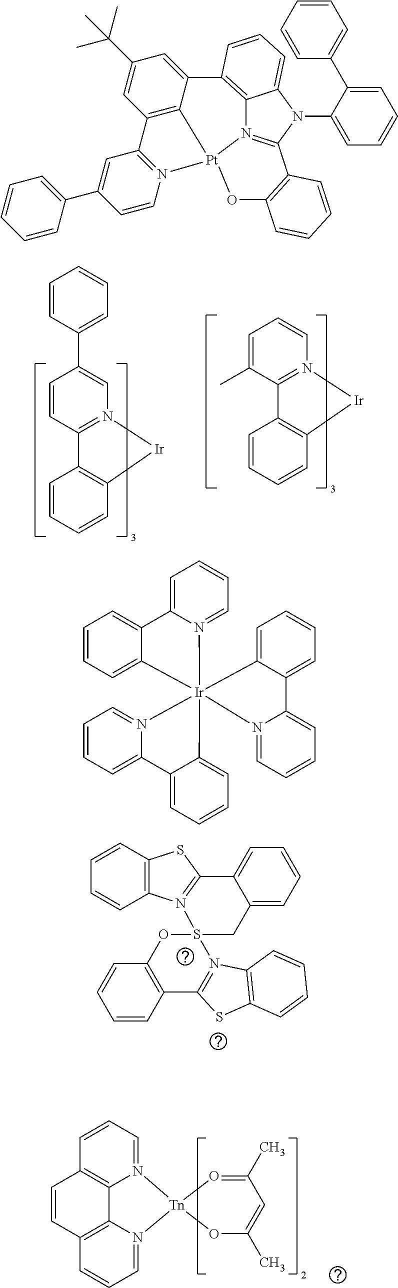

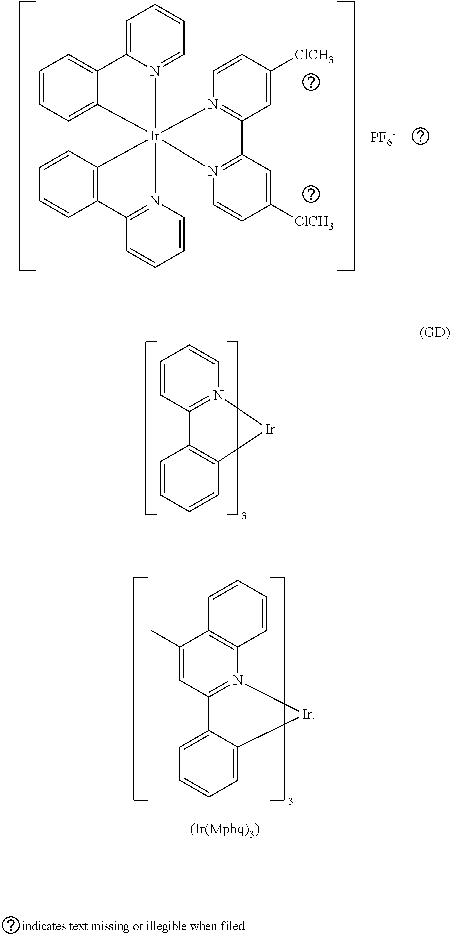

In one embodiment of the present application, the organic electroluminescent device is a red light organic electroluminescent device. In a more specific embodiment, the host material of the organic light emitting layer 330 comprises the nitrogen-containing compound of the present application. The guest material is, for example, RD-1 or GD.

In one embodiment of the present application, the organic electroluminescent device is a green light organic electroluminescent device. In a more specific embodiment, the host material of the organic light emitting layer 330 comprises the nitrogen-containing compound of the present application. The guest material is, for example, fac-Ir(ppy)₃.

The electron transport layer 340 may be a single-layer structure or a multi-layer structure, and may comprise one or more electron transport materials. The electron transport materials may be selected from, but are not limited to, BTB, LiQ, benzimidazole derivatives, oxadiazole derivatives, quinoxaline derivatives, or other electron transport materials, which is not particularly limited in the present application. The material of the electron transport layer 340 includes, but is not limited to, the following compounds:

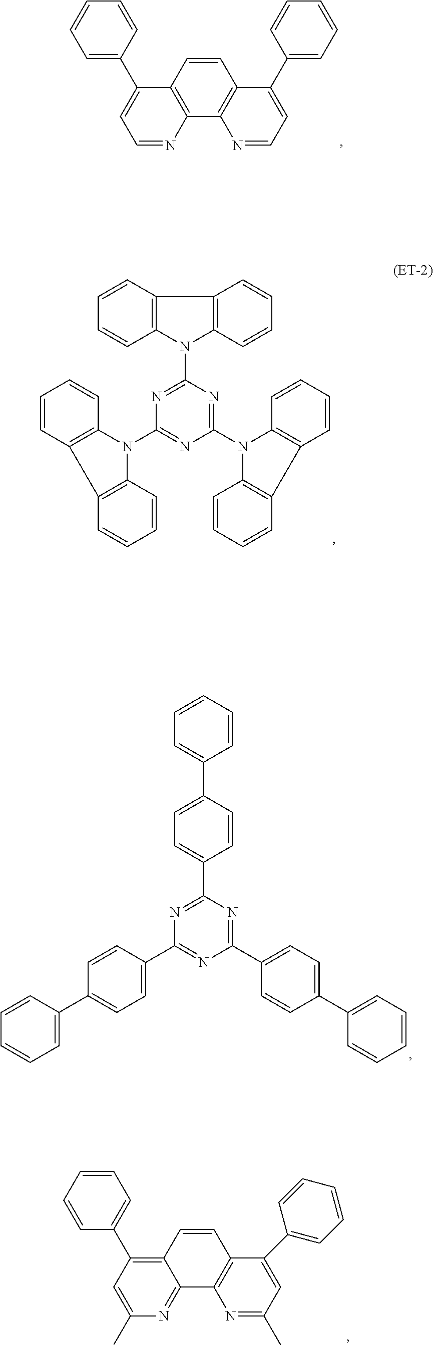 [SMILES: c1ccc(-c2ccnc3c2ccc2c(-c4ccccc4)ccnc23)cc1 ; c1ccc2c(c1)c1ccccc1n2-c1nc(-n2c3ccccc3c3ccccc32)nc(-n2c3ccccc3c3ccccc32)n1 ; c1ccc(-c2ccc(-c3nc(-c4ccc(-c5ccccc5)cc4)nc(-c4ccc(-c5ccccc5)cc4)n3)cc2)cc1 ; Cc1cc(-c2ccccc2)c2ccc3c(-c4ccccc4)cc(C)nc3c2n1]

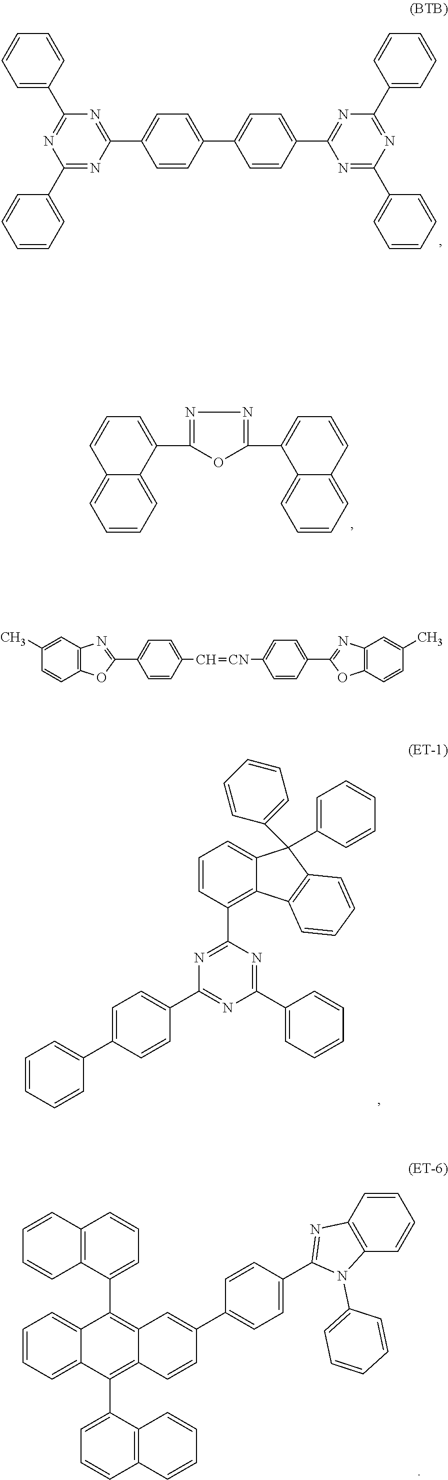

In one embodiment of the present application, the electron transport layer 340 may be composed of ET-1 and LiQ, or composed of ET-2 and LiQ, or composed of ET-6 and LiQ.

In the present application, the cathode 200 may comprise a cathode material, which is a small work function material contributing to injection of electrons into the functional layer. Specific examples of the cathode material include, but are not limited to, metals such as magnesium, calcium, sodium, potassium, titanium, indium, yttrium, lithium, gadolinium, aluminum, silver, tin, lead, and alloys thereof; or multilayer materials such as LiF/Al, Liq/Al, LiO₂/Al, LiF/Ca, LiF/Al, and BaF₂/Ca. Alternatively, a metal electrode comprising magnesium and silver is included as the cathode.

Alternatively, an electron injection layer 350 is further provided between the cathode 200 and the electron transport layer 340 so as to enhance the ability to inject electrons into the electron transport layer 340. The electron injection layer 350 may comprise an inorganic material such as an alkali metal sulfide, an alkali metal halide, and the like, or may include a complex of an alkali metal and an organic compound. In one embodiment of the present application, the electron injection layer 350 may comprise ytterbium (Yb).

In a third aspect, the present application provides an electronic apparatus comprising the organic electroluminescent device described in the second aspect of the present application.

According to one embodiment, as shown in FIG. 2, the electronic apparatus provided is an electronic apparatus 400 including the organic electroluminescent device described above. The electronic apparatus 400 may be, for example, a display device, a lighting device, an optical communication device or other type of electronic apparatus, including, but not limited to, for example, a computer screen, a mobile phone screen, a television, electronic paper, an emergency lamp, an optical module, etc.

A synthesis method of the nitrogen-containing compound of the present application is described in detail below in conjunction with synthesis examples, but the present application is not limited thereto in any way.

### SYNTHESIS EXAMPLES

Those skilled in the art should appreciate that chemical reactions described in the present application may be used properly to prepare many organic compounds of the present application, and other methods that can be used to prepare the compounds of the present application are all considered to be within the scope of the present application. For example, the synthesis of those non-exemplary compounds of the present application may be successfully accomplished by those skilled in the art by modifying the method, for example, by properly protecting an interfering group, by utilizing other known reagents other than those described in the present application, or by making some conventional modifications to reaction conditions. Compounds for which a synthesis method is not mentioned in the present application are raw material products obtained commercially.

### Synthesis of Sub-a1

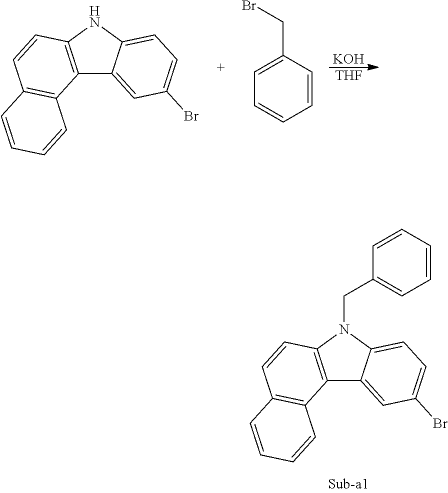

Under a nitrogen atmosphere, 10-bromo-7H-benzo[C]carbazole (29.62 g, 100 mmol), benzyl bromide (25.65 g, 150 mmol), potassium hydroxide (KOH, 11.22 g, 200 mmol) and tetrahydrofuran (THF, 300 mL) were added sequentially to a 500 mL of three-necked flask, stirred and heated to 60° C. for a reaction for 6 hours. After being cooled to room temperature, the system was extracted with tetrahydrofuran (100 mL×3 times). The resulting organic phases were combined and dried over anhydrous sodium sulfate, followed by filtration and vacuum distillation to remove the solvent, obtaining a crude product. The crude product was purified by silica gel column chromatography with n-heptane/dichloromethane as a mobile phase to yield Sub-a1 as a white solid (33.22 g, yield: 86%).

Sub-a2 to Sub-a10 were synthesized respectively with reference to the synthesis of Sub-a1, with 10-bromo-7H-benzo[C]carbazole being replaced with Reactant A shown in Table 1.

TABLE 1
Synthesis of Sub-a2 to Sub-a10

| Sub-a No. | Reactant A | Structure of Sub-a | Yield (%) |
|---|---|---|---|
| Sub-a2 | 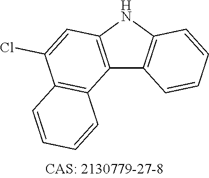 [SMILES: Clc1cc2[nH]c3ccccc3c2c2ccccc12] | 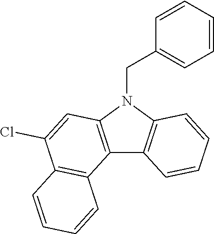 [SMILES: Clc1cc2c(c3ccccc13)c1ccccc1n2Cc1ccccc1] | 79 |
| Sub-a3 | 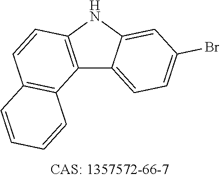 [SMILES: Brc1ccc2c(c1)[nH]c1ccc3ccccc3c12] | 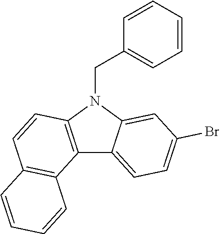 [SMILES: Brc1ccc2c3c4ccccc4ccc3n(Cc3ccccc3)c2c1] | 84 |
| Sub-a4 | 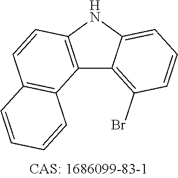 [SMILES: Brc1cccc2[nH]c3ccc4ccccc4c3c12] | 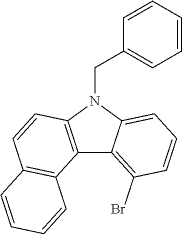 [SMILES: Brc1cccc2c1c1c3ccccc3ccc1n2Cc1ccccc1] | 85 |
| Sub-a5 | 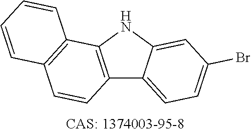 [SMILES: Brc1ccc2c(c1)[nH]c1c3ccccc3ccc21] | 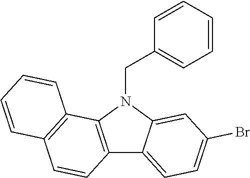 [SMILES: Brc1ccc2c3ccc4ccccc4c3n(Cc3ccccc3)c2c1] | 81 |
| Sub-a6 | 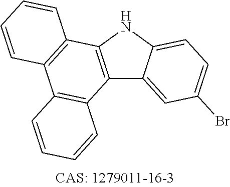 [SMILES: Brc1ccc2[nH]c3c4ccccc4c4ccccc4c3c2c1] | 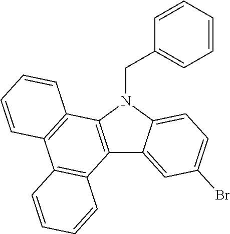 [SMILES: Brc1ccc2c(c1)c1c3ccccc3c3ccccc3c1n2Cc1ccccc1] | 82 |
| Sub-a7 | 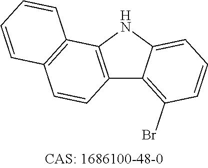 [SMILES: Brc1cccc2[nH]c3c4ccccc4ccc3c12] | 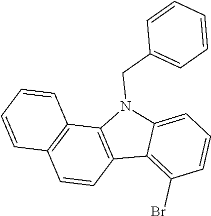 [SMILES: Brc1cccc2c1c1ccc3ccccc3c1n2Cc1ccccc1] | 81 |
| Sub-a8 | 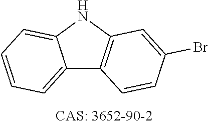 [SMILES: Brc1ccc2c(c1)[nH]c1ccccc12] | 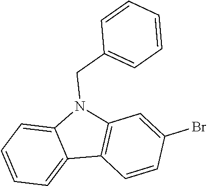 [SMILES: Brc1ccc2c3ccccc3n(Cc3ccccc3)c2c1] | 78 |
| Sub-a9 | 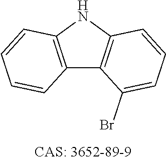 [SMILES: Brc1cccc2[nH]c3ccccc3c12] | 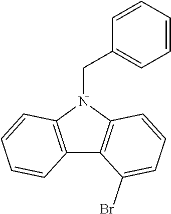 [SMILES: Brc1cccc2c1c1ccccc1n2Cc1ccccc1] | 76 |
| Sub-a10 | 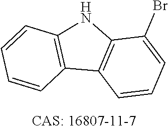 [SMILES: Brc1cccc2c1[nH]c1ccccc12] | 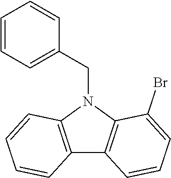 [SMILES: Brc1cccc2c3ccccc3n(Cc3ccccc3)c12] | 80 |

### Synthesis of Sub-b1

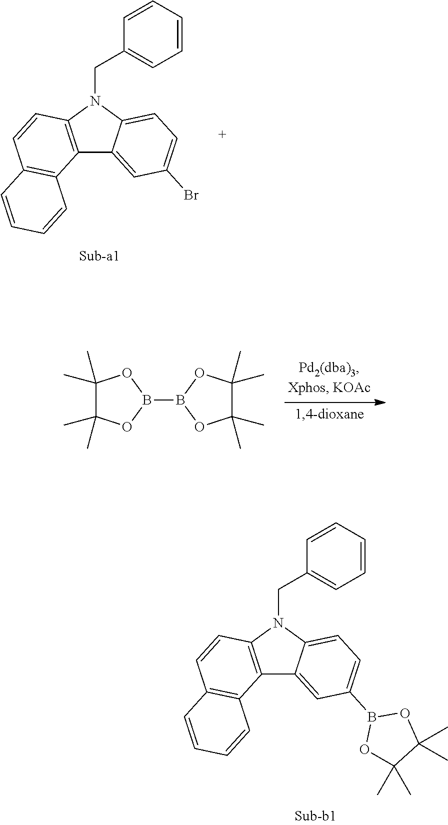

Under a nitrogen atmosphere, Sub-a1 (19.31 g, 50 mmol), bis(pinacolato)diboron (15.24 g, 60 mmol), potassium acetate (KOAc, 9.81 g, 100 mmol) and 1,4-dioxane (220 mL) were added sequentially to a 500 mL of three-necked flask, stirred and heated. When the system was heated to 40° C., tris(dibenzylideneacetone) dipalladium (Pd₂(dba)₂, 0.46 g, 0.5 mmol) and 2-dicyclohexylphosphino-2′,4′,6′-triisopropyl biphenyl (Xphos, 0.48 g, 1.0 mmol) were quickly added thereto. Heating was then continued to heat the system to a refluxing temperature for a reaction under stirring overnight. After the system was cooled to room temperature, 200 mL of water was added to the system. The resulting mixture was stirred thoroughly for 30 minutes, and filtered under reduced pressure. The resulting filter cake was washed with deionized water to neutral, and then rinsed with 100 mL of absolute ethanol to obtain a crude product as a gray solid. The crude product was triturated with n-heptane once, and then dissolved in 200 mL of toluene. After the resulting solution became clear, the solution was passed through a silica gel column to remove the catalyst, followed by concentration, yielding Sub-b1 as a white solid (16.46 g, yield: 76%).

Sub-b2 to Sub-b18 were synthesized respectively with reference to the synthesis of Sub-b1, with Sub-a1 being replaced with Reactant B shown in Table 2.

TABLE 2
Synthesis of Sub-b2 to Sub-b18

| Sub-b No. | Reactant B | Structure of Sub-b | Yield (%) |
|---|---|---|---|
| Sub-b2 | 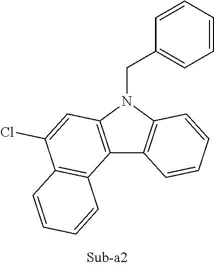 [SMILES: Clc1cc2c(c3ccccc13)c1ccccc1n2Cc1ccccc1] | 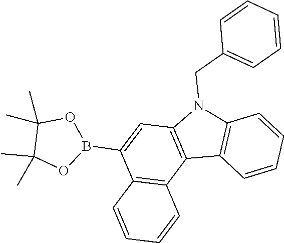 [SMILES: CC1(C)OB(c2cc3c(c4ccccc24)c2ccccc2n3Cc2ccccc2)OC1(C)C] | 73 |
| Sub-b3 | 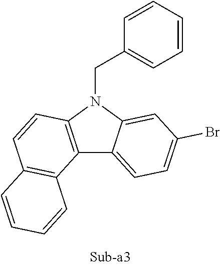 [SMILES: Brc1ccc2c3c4ccccc4ccc3n(Cc3ccccc3)c2c1] | 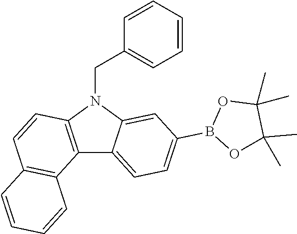 [SMILES: CC1(C)OB(c2ccc3c4c5ccccc5ccc4n(Cc4ccccc4)c3c2)OC1(C)C] | 76 |
| Sub-b4 | 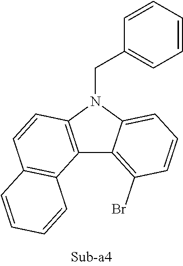 [SMILES: Brc1cccc2c1c1c3ccccc3ccc1n2Cc1ccccc1] | 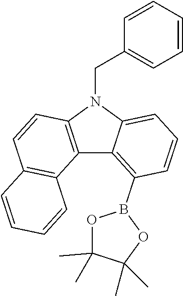 [SMILES: CC1(C)OB(c2cccc3c2c2c4ccccc4ccc2n3Cc2ccccc2)OC1(C)C] | 60 |
| Sub-b5 | 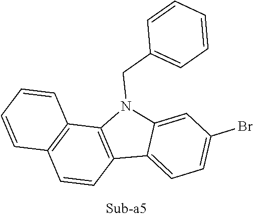 [SMILES: Brc1ccc2c3ccc4ccccc4c3n(Cc3ccccc3)c2c1] | 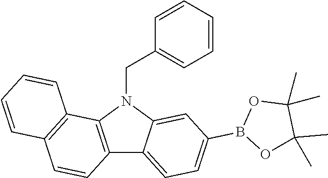 [SMILES: CC1(C)OB(c2ccc3c4ccc5ccccc5c4n(Cc4ccccc4)c3c2)OC1(C)C] | 73 |
| Sub-b6 | 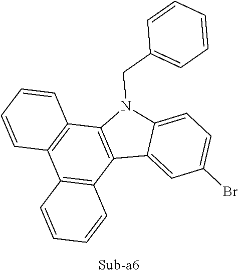 [SMILES: Brc1ccc2c(c1)c1c3ccccc3c3ccccc3c1n2Cc1ccccc1] | 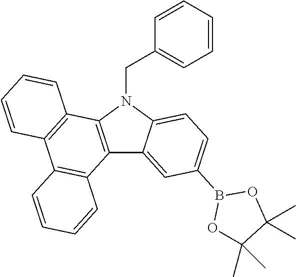 [SMILES: CC1(C)OB(c2ccc3c(c2)c2c4ccccc4c4ccccc4c2n3Cc2ccccc2)OC1(C)C] | 73 |
| Sub-b7 | 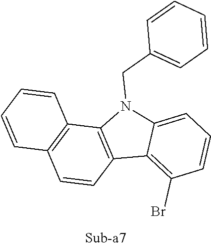 [SMILES: Brc1cccc2c1c1ccc3ccccc3c1n2Cc1ccccc1] | 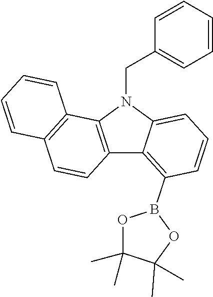 [SMILES: CC1(C)OB(c2cccc3c2c2ccc4ccccc4c2n3Cc2ccccc2)OC1(C)C] | 62 |
| Sub-b8 | 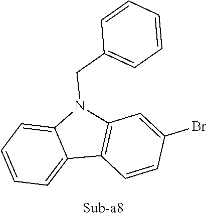 [SMILES: Brc1ccc2c3ccccc3n(Cc3ccccc3)c2c1] | 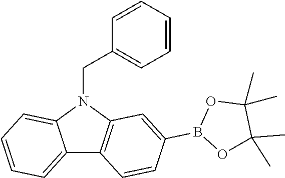 [SMILES: CC1(C)OB(c2ccc3c4ccccc4n(Cc4ccccc4)c3c2)OC1(C)C] | 61 |
| Sub-b9 | 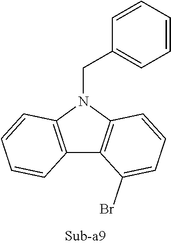 [SMILES: Brc1cccc2c1c1ccccc1n2Cc1ccccc1] | 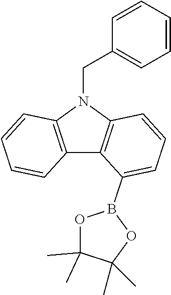 [SMILES: CC1(C)OB(c2cccc3c2c2ccccc2n3Cc2ccccc2)OC1(C)C] | 58 |
| Sub-b10 | 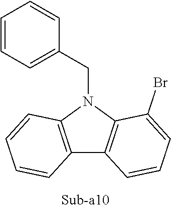 [SMILES: Brc1cccc2c3ccccc3n(Cc3ccccc3)c12] | 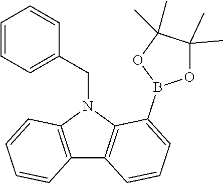 [SMILES: CC1(C)OB(c2cccc3c4ccccc4n(Cc4ccccc4)c23)OC1(C)C] | 54 |
| Sub-b11 | 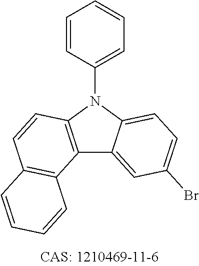 [SMILES: Brc1ccc2c(c1)c1c3ccccc3ccc1n2-c1ccccc1] | 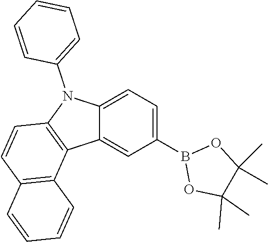 [SMILES: CC1(C)OB(c2ccc3c(c2)c2c4ccccc4ccc2n3-c2ccccc2)OC1(C)C] | 68 |
| Sub-b12 | 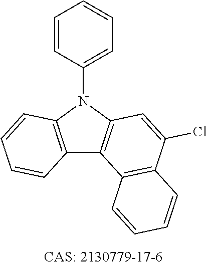 [SMILES: Clc1cc2c(c3ccccc13)c1ccccc1n2-c1ccccc1] | 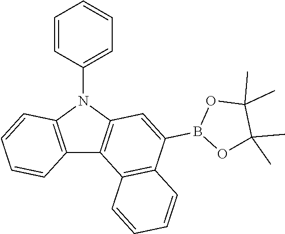 [SMILES: CC1(C)OB(c2cc3c(c4ccccc24)c2ccccc2n3-c2ccccc2)OC1(C)C] | 71 |
| Sub-b13 | 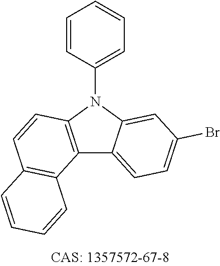 [SMILES: Brc1ccc2c3c4ccccc4ccc3n(-c3ccccc3)c2c1] | 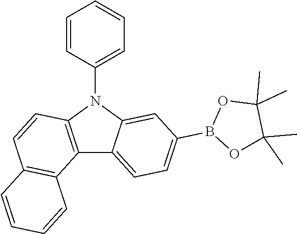 [SMILES: CC1(C)OB(c2ccc3c4c5ccccc5ccc4n(-c4ccccc4)c3c2)OC1(C)C] | 61 |
| Sub-b14 | 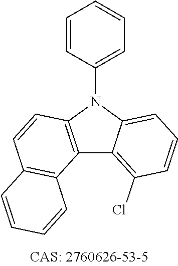 [SMILES: Clc1cccc2c1c1c3ccccc3ccc1n2-c1ccccc1] | 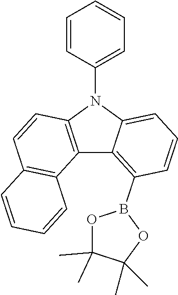 [SMILES: CC1(C)OB(c2cccc3c2c2c4ccccc4ccc2n3-c2ccccc2)OC1(C)C] | 59 |
| Sub-b15 | 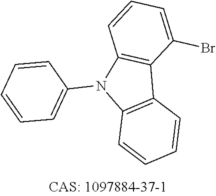 [SMILES: Brc1cccc2c1c1ccccc1n2-c1ccccc1] | 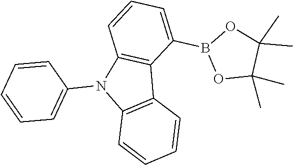 [SMILES: CC1(C)OB(c2cccc3c2c2ccccc2n3-c2ccccc2)OC1(C)C] | 65 |
| Sub-b16 | 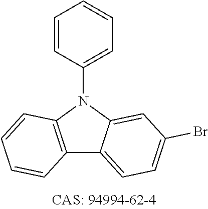 [SMILES: Brc1ccc2c3ccccc3n(-c3ccccc3)c2c1] | 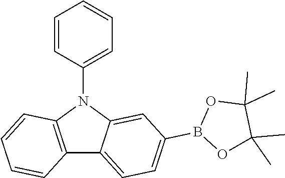 [SMILES: CC1(C)OB(c2ccc3c4ccccc4n(-c4ccccc4)c3c2)OC1(C)C] | 64 |
| Sub-b17 | 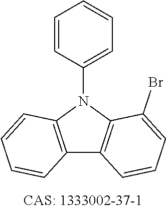 [SMILES: Brc1cccc2c3ccccc3n(-c3ccccc3)c12] | 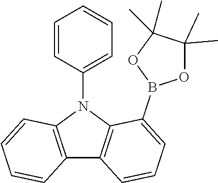 [SMILES: CC1(C)OB(c2cccc3c4ccccc4n(-c4ccccc4)c23)OC1(C)C] | 62 |
| Sub-b18 | 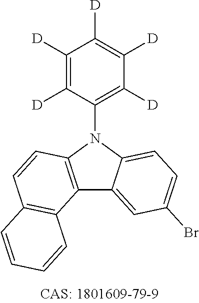 [SMILES: [2H]c1c([2H])c([2H])c(-n2c3ccc(Br)cc3c3c4ccccc4ccc32)c([2H])c1[2H]] | 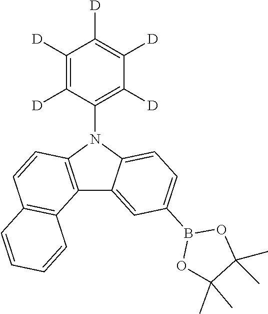 [SMILES: [2H]c1c([2H])c([2H])c(-n2c3ccc(B4OC(C)(C)C(C)(C)O4)cc3c3c4ccccc4ccc32)c([2H])c1[2H]] | 71 |

### Synthesis of Sub-c1

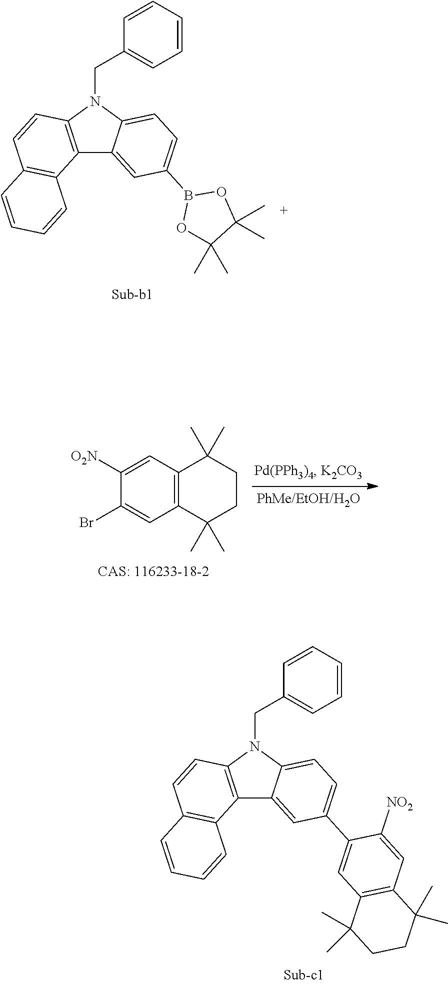 [SMILES: CC1(C)OB(c2ccc3c(c2)c2c4ccccc4ccc2n3Cc2ccccc2)OC1(C)C ; CC1(C)CCC(C)(C)c2cc([N+](=O)[O-])c(Br)cc21 ; CC1(C)CCC(C)(C)c2cc([N+](=O)[O-])c(-c3ccc4c(c3)c3c5ccccc5ccc3n4Cc3ccccc3)cc21]

Under a nitrogen atmosphere, Sub-b1 (23.83 g, 55 mmol), CAS:116233-18-2 (15.61 g, 50 mmol), tetrakis(triphenylphosphino)palladium (Pd(PPh₃)₄, 0.58 g, 0.5 mmol), anhydrous potassium carbonate (K₂CO₃, 13.82 g, 100 mmol), toluene (PhMe, 240 mL), absolute ethanol (EtOH, 60 mL) and deionized water (H₂O, 60 mL) were added sequentially to a 1000 mL of three-necked flask, stirred and heated to a refluxing temperature for a reaction for 16 hours. After being cooled to room temperature, the system was extracted with dichloromethane (100 mL×3 times). The resulting organic phases were combined and dried over anhydrous sodium sulfate, followed by filtration and vacuum distillation to remove the solvent to obtain a crude product. The crude product was purified by silica column chromatography with n-heptane/dichloromethane as a mobile phase to yield Sub-c1 as a white solid (21.0 g, yield: 78%).

Sub-c2 to Sub-c18 were synthesized respectively with reference to the synthesis of Sub-c1, with Sub-b1 being replaced with Reactant C shown in Table 3.

TABLE 3
Synthesis of Sub-c2 to Sub-18

| Sub-c No. | Reactant C | Structure of Sub-c | Yield (%) |
|---|---|---|---|
| Sub-c2 | 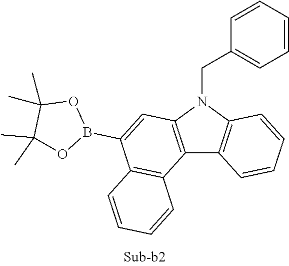 [SMILES: CC1(C)OB(c2cc3c(c4ccccc24)c2ccccc2n3Cc2ccccc2)OC1(C)C] | 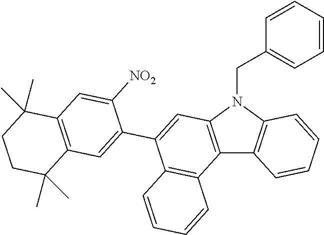 [SMILES: CC1(C)CCC(C)(C)c2cc([N+](=O)[O-])c(-c3cc4c(c5ccccc35)c3ccccc3n4Cc3ccccc3)cc21] | 75 |
| Sub-c3 | 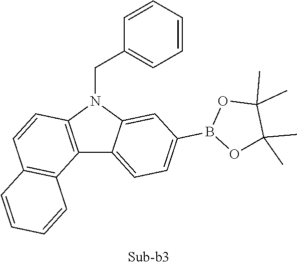 [SMILES: CC1(C)OB(c2ccc3c4c5ccccc5ccc4n(Cc4ccccc4)c3c2)OC1(C)C] | 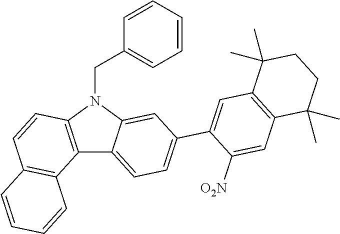 [SMILES: CC1(C)CCC(C)(C)c2cc([N+](=O)[O-])c(-c3ccc4c5c6ccccc6ccc5n(Cc5ccccc5)c4c3)cc21] | 78 |
| Sub-c4 | 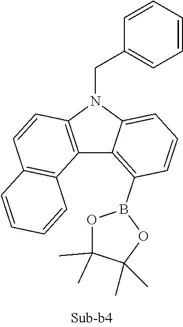 [SMILES: CC1(C)OB(c2cccc3c2c2c4ccccc4ccc2n3Cc2ccccc2)OC1(C)C] | 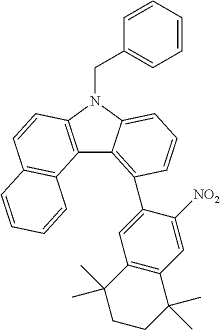 [SMILES: CC1(C)CCC(C)(C)c2cc([N+](=O)[O-])c(-c3cccc4c3c3c5ccccc5ccc3n4Cc3ccccc3)cc21] | 64 |
| Sub-c5 | 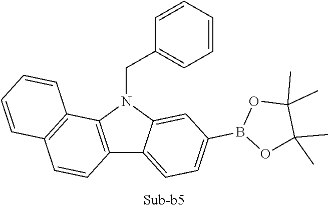 [SMILES: CC1(C)OB(c2ccc3c4ccc5ccccc5c4n(Cc4ccccc4)c3c2)OC1(C)C] | 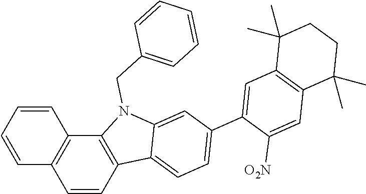 [SMILES: CC1(C)CCC(C)(C)c2cc([N+](=O)[O-])c(-c3ccc4c5ccc6ccccc6c5n(Cc5ccccc5)c4c3)cc21] | 73 |
| Sub-c6 | 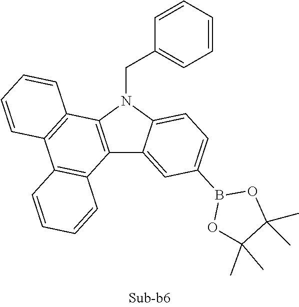 [SMILES: CC1(C)OB(c2ccc3c(c2)c2c4ccccc4c4ccccc4c2n3Cc2ccccc2)OC1(C)C] | 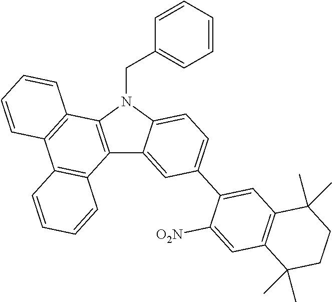 [SMILES: CC1(C)CCC(C)(C)c2cc([N+](=O)[O-])c(-c3ccc4c(c3)c3c5ccccc5c5ccccc5c3n4Cc3ccccc3)cc21] | 74 |
| Sub-c7 | 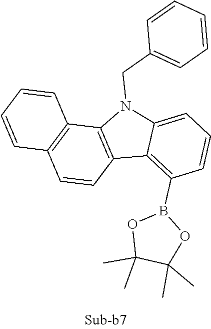 [SMILES: CC1(C)OB(c2cccc3c2c2ccc4ccccc4c2n3Cc2ccccc2)OC1(C)C] | 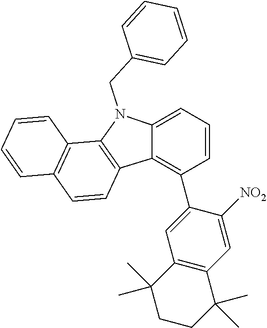 [SMILES: CC1(C)CCC(C)(C)c2cc([N+](=O)[O-])c(-c3cccc4c3c3ccc5ccccc5c3n4Cc3ccccc3)cc21] | 65 |
| Sub-c8 | 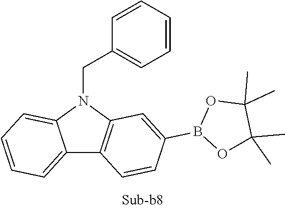 [SMILES: CC1(C)OB(c2ccc3c4ccccc4n(Cc4ccccc4)c3c2)OC1(C)C] | 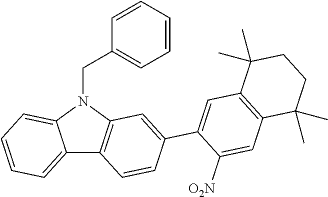 [SMILES: CC1(C)CCC(C)(C)c2cc([N+](=O)[O-])c(-c3ccc4c5ccccc5n(Cc5ccccc5)c4c3)cc21] | 71 |
| Sub-c9 | 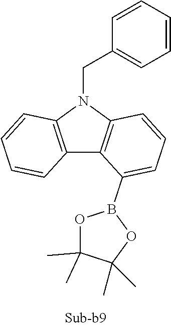 [SMILES: CC1(C)OB(c2cccc3c2c2ccccc2n3Cc2ccccc2)OC1(C)C] | 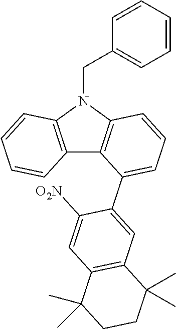 [SMILES: CC1(C)CCC(C)(C)c2cc([N+](=O)[O-])c(-c3cccc4c3c3ccccc3n4Cc3ccccc3)cc21] | 64 |
| Sub-c10 | 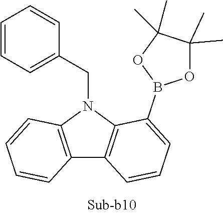 [SMILES: CC1(C)OB(c2cccc3c4ccccc4n(Cc4ccccc4)c23)OC1(C)C] | 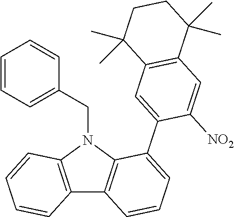 [SMILES: CC1(C)CCC(C)(C)c2cc([N+](=O)[O-])c(-c3cccc4c5ccccc5n(Cc5ccccc5)c34)cc21] | 63 |
| Sub-c11 | 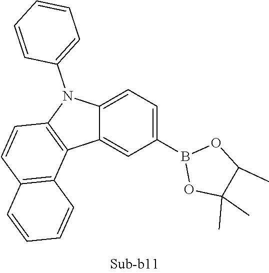 [SMILES: CC1OB(c2ccc3c(c2)c2c4ccccc4ccc2n3-c2ccccc2)OC1(C)C] | 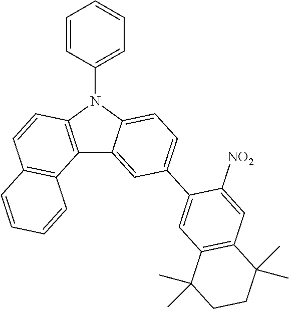 [SMILES: CC1(C)CCC(C)(C)c2cc([N+](=O)[O-])c(-c3ccc4c(c3)c3c5ccccc5ccc3n4-c3ccccc3)cc21] | 75 |
| Sub-c12 | 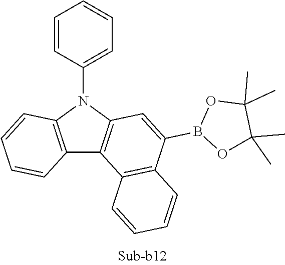 [SMILES: CC1(C)OB(c2cc3c(c4ccccc24)c2ccccc2n3-c2ccccc2)OC1(C)C] | 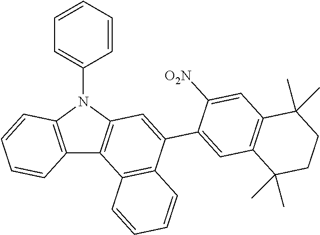 [SMILES: CC1(C)CCC(C)(C)c2cc([N+](=O)[O-])c(-c3cc4c(c5ccccc35)c3ccccc3n4-c3ccccc3)cc21] | 76 |
| Sub-c13 | 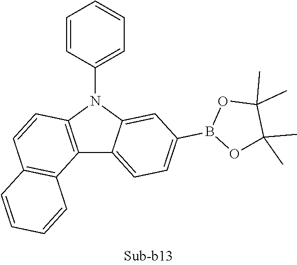 [SMILES: CC1(C)OB(c2ccc3c4c5ccccc5ccc4n(-c4ccccc4)c3c2)OC1(C)C] | 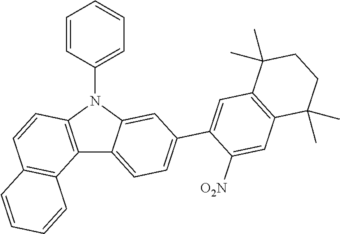 [SMILES: CC1(C)CCC(C)(C)c2cc([N+](=O)[O-])c(-c3ccc4c5c6ccccc6ccc5n(-c5ccccc5)c4c3)cc21] | 70 |
| Sub-c14 | 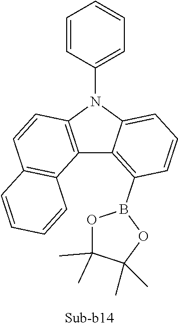 [SMILES: CC1(C)OB(c2cccc3c2c2c4ccccc4ccc2n3-c2ccccc2)OC1(C)C] | 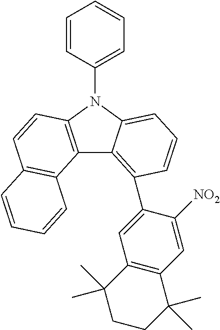 [SMILES: CC1(C)CCC(C)(C)c2cc([N+](=O)[O-])c(-c3cccc4c3c3c5ccccc5ccc3n4-c3ccccc3)cc21] | 61 |
| Sub-c15 | 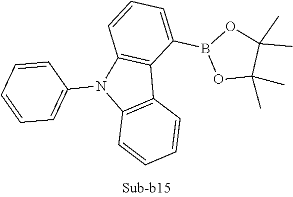 [SMILES: CC1(C)OB(c2cccc3c2c2ccccc2n3-c2ccccc2)OC1(C)C] | 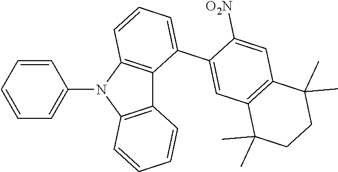 [SMILES: CC1(C)CCC(C)(C)c2cc([N+](=O)[O-])c(-c3cccc4c3c3ccccc3n4-c3ccccc3)cc21] | 63 |
| Sub-c16 | 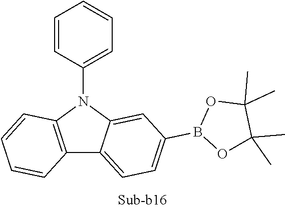 [SMILES: CC1(C)OB(c2ccc3c4ccccc4n(-c4ccccc4)c3c2)OC1(C)C] | 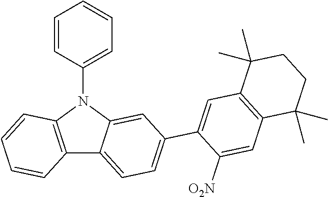 [SMILES: CC1(C)CCC(C)(C)c2cc([N+](=O)[O-])c(-c3ccc4c5ccccc5n(-c5ccccc5)c4c3)cc21] | 79 |
| Sub-c17 | 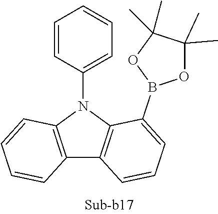 [SMILES: CC1(C)OB(c2cccc3c4ccccc4n(-c4ccccc4)c23)OC1(C)C] | 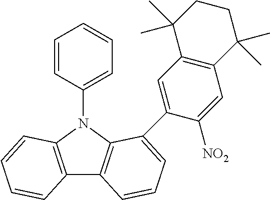 [SMILES: CC1(C)CCC(C)(C)c2cc([N+](=O)[O-])c(-c3cccc4c5ccccc5n(-c5ccccc5)c34)cc21] | 58 |
| Sub-c18 | 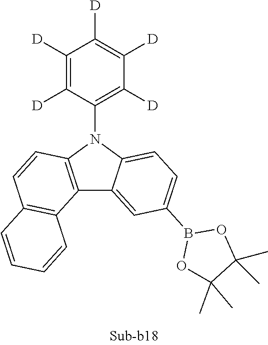 [SMILES: [2H]c1c([2H])c([2H])c(-n2c3ccc(B4OC(C)(C)C(C)(C)O4)cc3c3c4ccccc4ccc32)c([2H])c1[2H]] | 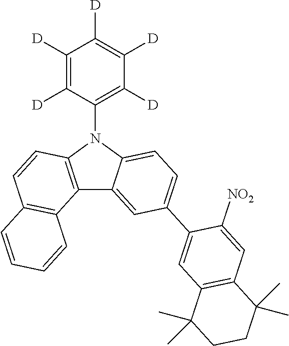 [SMILES: [2H]c1c([2H])c([2H])c(-n2c3ccc(-c4cc5c(cc4[N+](=O)[O-])C(C)(C)CCC5(C)C)cc3c3c4ccccc4ccc32)c([2H])c1[2H]] | 68 |

### Synthesis of Sub-d1

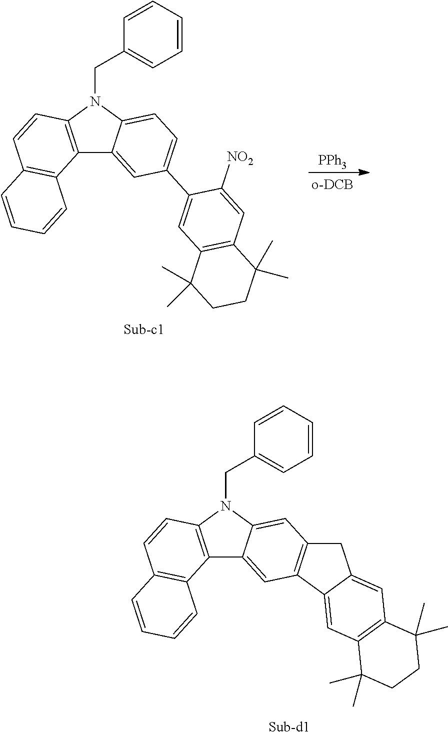 [SMILES: CC1(C)CCC(C)(C)c2cc([N+](=O)[O-])c(-c3ccc4c(c3)c3c5ccccc5ccc3n4Cc3ccccc3)cc21 ; CC1(C)CCC(C)(C)c2cc3c(cc21)Cc1cc2c(cc1-3)c1c3ccccc3ccc1n2Cc1ccccc1 ; "PPh3"]

Under a nitrogen atmosphere, Sub-c1 (26.93 g, 50 mmol), triphenylphosphine (PPh₃, 32.78 g, 125 mmol) and o-dichlorobenzene (o-DCB, 150 mL) were added sequentially to a 250 mL of three-necked flask, stirred and heated to a refluxing temperature for a reaction for 16 hours. After being cooled to room temperature, the system was subjected to vacuum distillation to remove the solvent, obtaining a crude product. The crude product was purified by silica column chromatography with n-heptane as a mobile phase to yield Sub-d1 as a gray-green solid (14.18 g, yield: 56%).

Sub-d2 to Sub-d18 were synthesized respectively with reference to the synthesis of Sub-d1, with Sub-c1 being replaced with Reactant D shown in Table 4.

TABLE 4
Synthesis of Sub-d2 to Sub-d18

| Sub-d | | | Yield |
|---|---|---|---|
| No. | Reactant D | Structure of Sub-d | (%) |
| Sub-d2 | 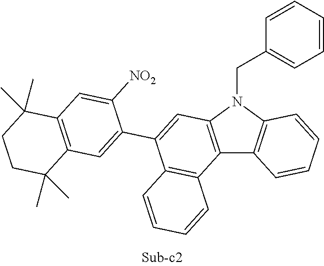 [SMILES: CC1(C)CCC(C)(C)c2cc([N+](=O)[O-])c(-c3cc4c(c5ccccc35)c3ccccc3n4Cc3ccccc3)cc21] | 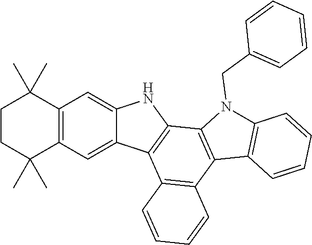 [SMILES: CC1(C)CCC(C)(C)c2cc3c(cc21)[nH]c1c3c2ccccc2c2c3ccccc3n(Cc3ccccc3)c12] | 53 |
| Sub-d3 | 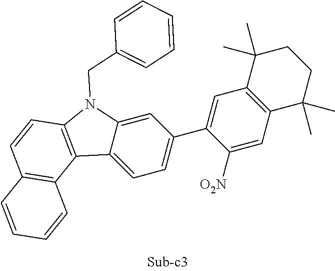 [SMILES: CC1(C)CCC(C)(C)c2cc([N+](=O)[O-])c(-c3ccc4c5c6ccccc6ccc5n(Cc5ccccc5)c4c3)cc21] | 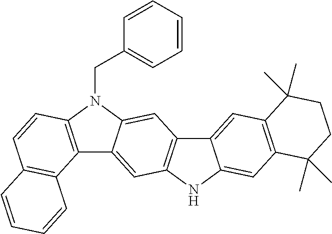 [SMILES: CC1(C)CCC(C)(C)c2cc3c(cc21)[nH]c1cc2c4c5ccccc5ccc4n(Cc4ccccc4)c2cc13] | 52 |
| Sub-d4 | 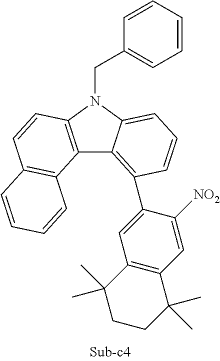 [SMILES: CC1(C)CCC(C)(C)c2cc([N+](=O)[O-])c(-c3cccc4c3c3c5ccccc5ccc3n4Cc3ccccc3)cc21] | 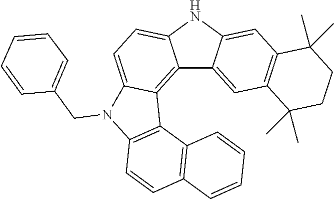 [SMILES: CC1(C)CCC(C)(C)c2cc3c(cc21)[nH]c1ccc2c(c13)c1c3ccccc3ccc1n2Cc1ccccc1] | 52 |
| Sub-d5 | 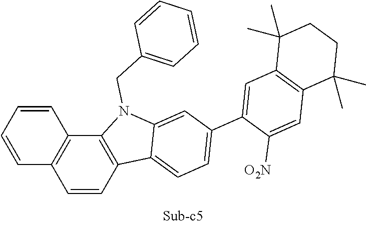 [SMILES: CC1(C)CCC(C)(C)c2cc([N+](=O)[O-])c(-c3ccc4c5ccc6ccccc6c5n(Cc5ccccc5)c4c3)cc21] | 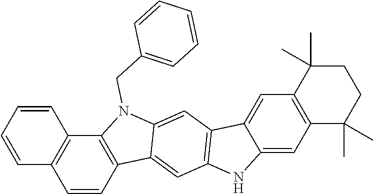 [SMILES: CC1(C)CCC(C)(C)c2cc3c(cc21)[nH]c1cc2c4ccc5ccccc5c4n(Cc4ccccc4)c2cc13] | 52 |
| Sub-d6 | 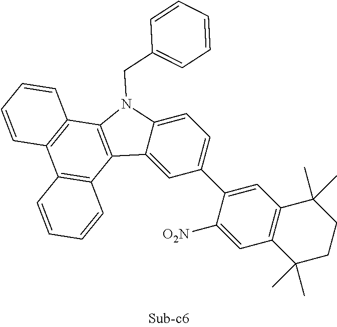 [SMILES: CC1(C)CCC(C)(C)c2cc([N+](=O)[O-])c(-c3ccc4c(c3)c3c5ccccc5c5ccccc5c3n4Cc3ccccc3)cc21] | 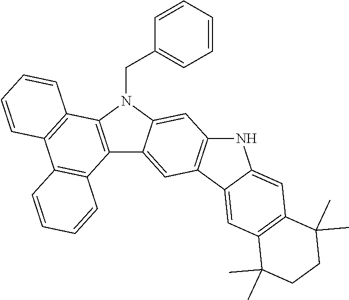 [SMILES: CC1(C)CCC(C)(C)c2cc3c(cc21)[nH]c1cc2c(cc13)c1c3ccccc3c3ccccc3c1n2Cc1ccccc1] | 51 |
| Sub-d7 | 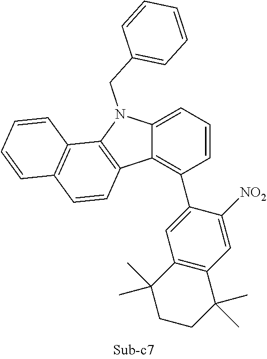 [SMILES: CC1(C)CCC(C)(C)c2cc([N+](=O)[O-])c(-c3cccc4c3c3ccc5ccccc5c3n4Cc3ccccc3)cc21] | 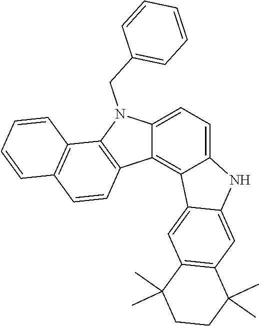 [SMILES: CC1(C)CCC(C)(C)c2cc3c(cc21)[nH]c1ccc2c(c4ccc5ccccc5c4n2Cc2ccccc2)c13] | 54 |
| Sub-d8 | 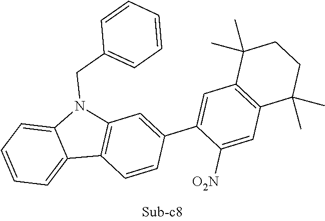 [SMILES: CC1(C)CCC(C)(C)c2cc([N+](=O)[O-])c(-c3ccc4c5ccccc5n(Cc5ccccc5)c4c3)cc21] | 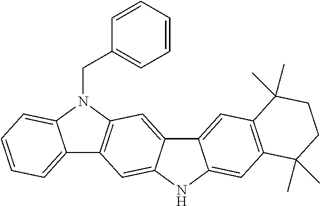 [SMILES: CC1(C)CCC(C)(C)c2cc3c(cc21)[nH]c1cc2c4ccccc4n(Cc4ccccc4)c2cc13] | 55 |
| Sub-d9 | 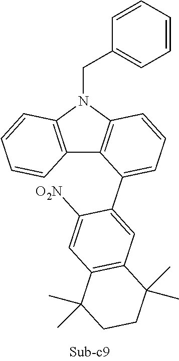 [SMILES: CC1(C)CCC(C)(C)c2cc([N+](=O)[O-])c(-c3cccc4c3c3ccccc3n4Cc3ccccc3)cc21] | 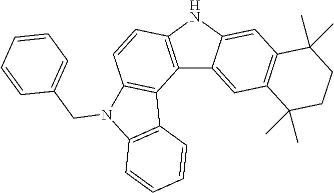 [SMILES: CC1(C)CCC(C)(C)c2cc3c(cc21)[nH]c1ccc2c(c4ccccc4n2Cc2ccccc2)c13] | 53 |
| Sub-d10 | 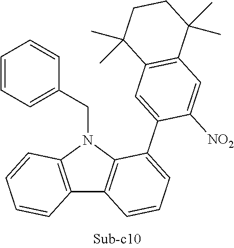 [SMILES: CC1(C)CCC(C)(C)c2cc([N+](=O)[O-])c(-c3cccc4c5ccccc5n(Cc5ccccc5)c34)cc21] | 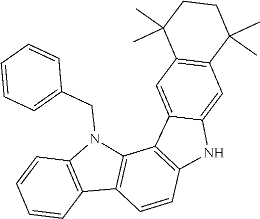 [SMILES: CC1(C)CCC(C)(C)c2cc3c(cc21)[nH]c1ccc2c4ccccc4n(Cc4ccccc4)c2c13] | 52 |
| Sub-d11 | 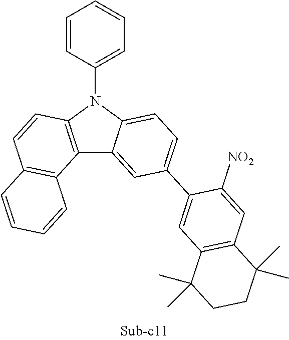 [SMILES: CC1(C)CCC(C)(C)c2cc([N+](=O)[O-])c(-c3ccc4c(c3)c3c5ccccc5ccc3n4-c3ccccc3)cc21] | 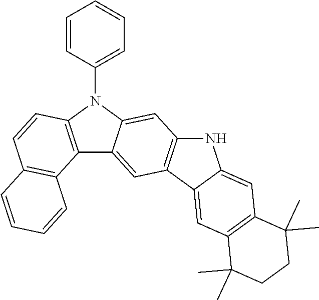 [SMILES: CC1(C)CCC(C)(C)c2cc3c(cc21)[nH]c1cc2c(cc13)c1c3ccccc3ccc1n2-c1ccccc1] | 54 |
| Sub-d12 | 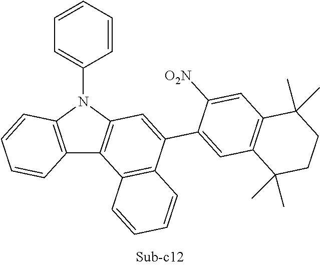 [SMILES: CC1(C)CCC(C)(C)c2cc([N+](=O)[O-])c(-c3cc4c(c5ccccc35)c3ccccc3n4-c3ccccc3)cc21] | 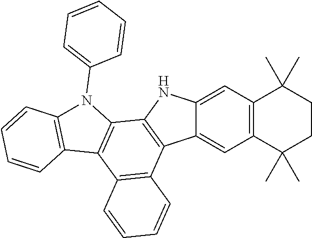 [SMILES: CC1(C)CCC(C)(C)c2cc3c(cc21)[nH]c1c3c2ccccc2c2c3ccccc3n(-c3ccccc3)c12] | 52 |
| Sub-d13 | 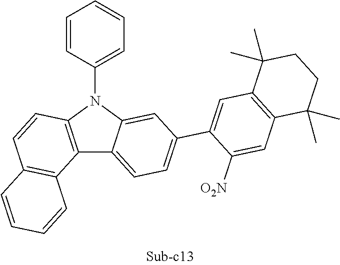 [SMILES: CC1(C)CCC(C)(C)c2cc([N+](=O)[O-])c(-c3ccc4c5c6ccccc6ccc5n(-c5ccccc5)c4c3)cc21] | 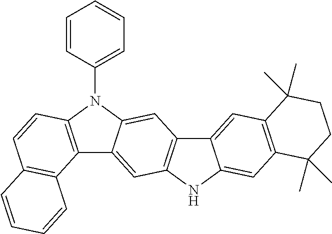 [SMILES: CC1(C)CCC(C)(C)c2cc3c(cc21)[nH]c1cc2c4c5ccccc5ccc4n(-c4ccccc4)c2cc13] | 52 |
| Sub-d14 | 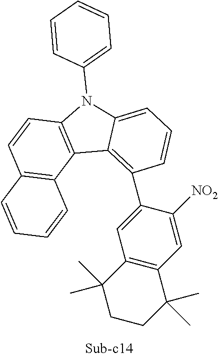 [SMILES: CC1(C)CCC(C)(C)c2cc([N+](=O)[O-])c(-c3cccc4c3c3c5ccccc5ccc3n4-c3ccccc3)cc21] | 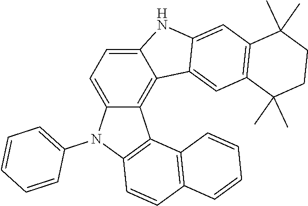 [SMILES: CC1(C)CCC(C)(C)c2cc3c(cc21)[nH]c1ccc2c(c13)c1c3ccccc3ccc1n2-c1ccccc1] | 51 |
| Sub-d15 | 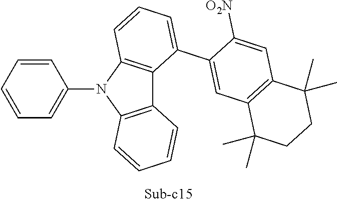 [SMILES: CC1(C)CCC(C)(C)c2cc([N+](=O)[O-])c(-c3cccc4c3c3ccccc3n4-c3ccccc3)cc21] | 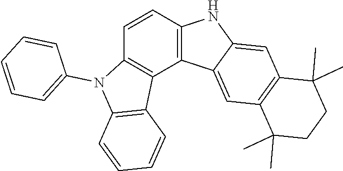 [SMILES: CC1(C)CCC(C)(C)c2cc3c(cc21)[nH]c1ccc2c(c4ccccc4n2-c2ccccc2)c13] | 52 |
| Sub-d16 | 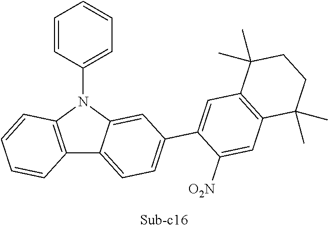 [SMILES: CC1(C)CCC(C)(C)c2cc([N+](=O)[O-])c(-c3ccc4c5ccccc5n(-c5ccccc5)c4c3)cc21] | 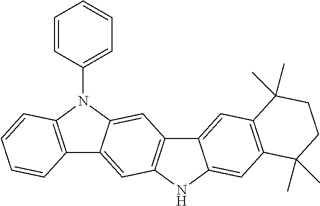 [SMILES: CC1(C)CCC(C)(C)c2cc3c(cc21)[nH]c1cc2c4ccccc4n(-c4ccccc4)c2cc13] | 50 |
| Sub-d17 | 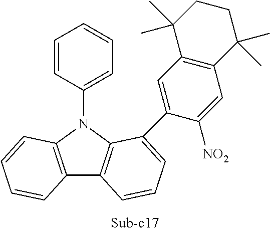 [SMILES: CC1(C)CCC(C)(C)c2cc([N+](=O)[O-])c(-c3cccc4c5ccccc5n(-c5ccccc5)c34)cc21] | 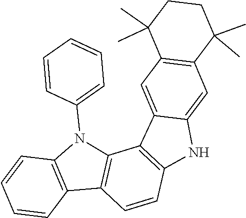 [SMILES: CC1(C)CCC(C)(C)c2cc3c(cc21)[nH]c1ccc2c4ccccc4n(-c4ccccc4)c2c13] | 51 |
| Sub-d18 | 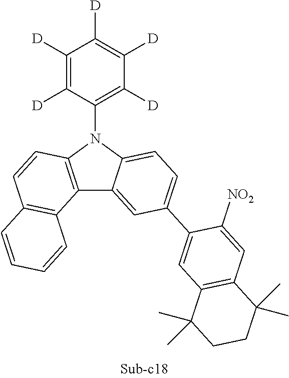 [SMILES: [2H]c1c([2H])c([2H])c(-n2c3ccc(-c4cc5c(cc4[N+](=O)[O-])C(C)(C)CCC5(C)C)cc3c3c4ccccc4ccc32)c([2H])c1[2H]] | 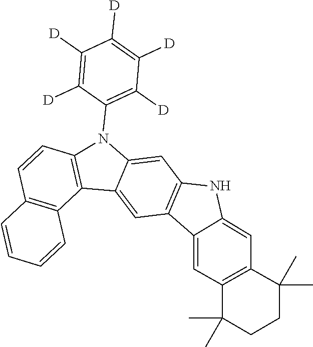 [SMILES: [2H]c1c([2H])c([2H])c(-n2c3cc4[nH]c5cc6c(cc5c4cc3c3c4ccccc4ccc32)C(C)(C)CCC6(C)C)c([2H])c1[2H]] | 52 |

### Synthesis of Sub-e1

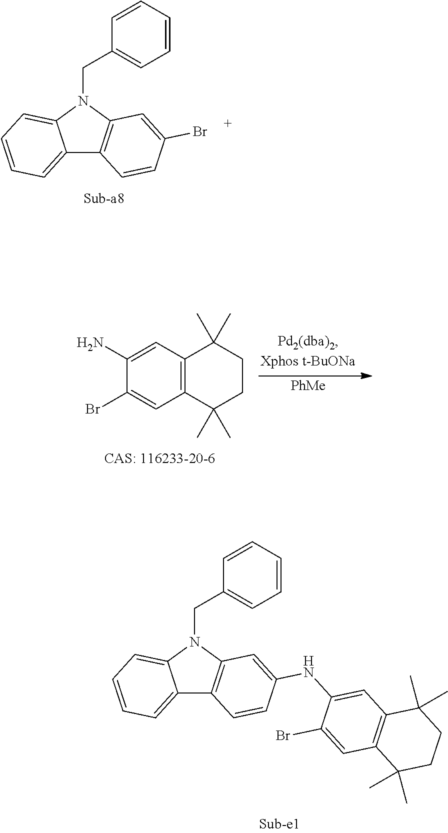 [SMILES: Brc1ccc2c3ccccc3n(Cc3ccccc3)c2c1 ; CC1(C)CCC(C)(C)c2cc(Br)c(N)cc21 ; "PhMe" ; CC1(C)CCC(C)(C)c2cc(Nc3ccc4c5ccccc5n(Cc5ccccc5)c4c3)c(Br)cc21]

Under a nitrogen atmosphere, Sub-a8 (16.81 g, 50 mmol), CAS:116233-20-6 (14.11 g, 50 mmol), tris (dibenzylideneacetone)palladium (Pd₂(dba)₂, 0.916 g, 1 mmol), 2-dicyclohexyl phosphino-2′,4′,6′-triisopropylbiphenyl (Xphos. 0.95 g. 2 mmol), sodium tert-butoxide (t-BuONa, 9.61 g, 100 mmol) and toluene (250 mL) were added sequentially to a 500 mL of three-necked flask, and heated to a refluxing temperature for a reaction under stirring overnight. After being cooled to room temperature, the system was extracted with dichloromethane (100 mL×3 times). The resulting organic phases were combined and dried over anhydrous sodium sulfate, followed by filtration and vacuum distillation to remove the solvent, obtaining a crude product. The crude product was purified by silica column chromatography with n-heptane/dichloromethane as a mobile phase to yield Sub-e1 as a gray-green solid (14.24 g, yield: 53%).

Sub-e2 to Sub-e7 were synthesized respectively with reference to the synthesis of Sub-e1, with Sub-a8 being replaced with Reactant E shown in Table 5.

TABLE 5
Synthesis of Sub-e2 to Sub-e7

| Sub-e No. | Reactant E | Structure of Sub-e | Yield (%) |
|---|---|---|---|
| Sub-e2 | 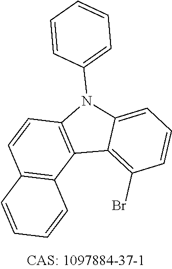 [SMILES: Brc1cccc2c1c1c3ccccc3ccc1n2-c1ccccc1] | 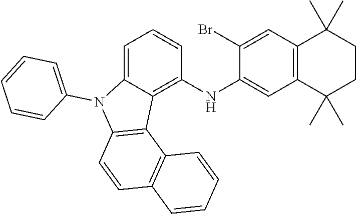 [SMILES: CC1(C)CCC(C)(C)c2cc(Nc3cccc4c3c3c5ccccc5ccc3n4-c3ccccc3)c(Br)cc21] | 54 |
| Sub-e3 | 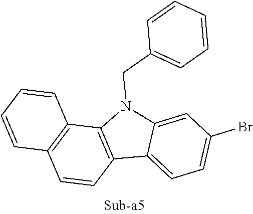 [SMILES: Brc1ccc2c3ccc4ccccc4c3n(Cc3ccccc3)c2c1] | 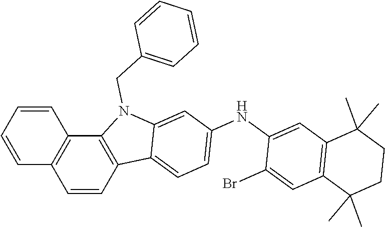 [SMILES: CC1(C)CCC(C)(C)c2cc(Nc3ccc4c5ccc6ccccc6c5n(Cc5ccccc5)c4c3)c(Br)cc21] | 45 |
| Sub-e4 | 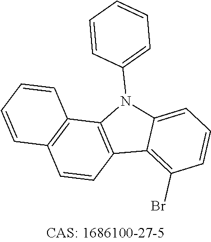 [SMILES: Brc1cccc2c1c1ccc3ccccc3c1n2-c1ccccc1] | 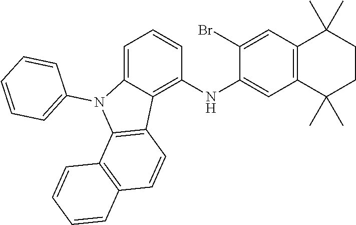 [SMILES: CC1(C)CCC(C)(C)c2cc(Nc3cccc4c3c3ccc5ccccc5c3n4-c3ccccc3)c(Br)cc21] | 50 |
| Sub-e5 | 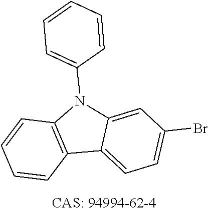 [SMILES: Brc1ccc2c3ccccc3n(-c3ccccc3)c2c1] | 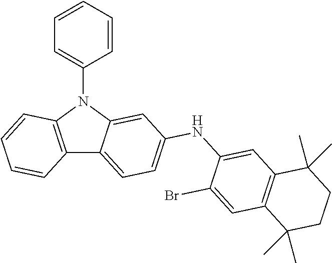 [SMILES: CC1(C)CCC(C)(C)c2cc(Nc3ccc4c5ccccc5n(-c5ccccc5)c4c3)c(Br)cc21] | 54 |
| Sub-e6 | 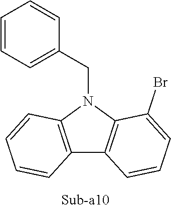 [SMILES: Brc1cccc2c3ccccc3n(Cc3ccccc3)c12] | 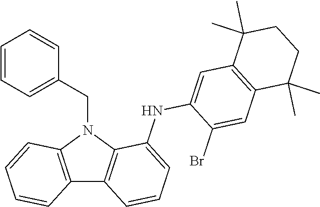 [SMILES: CC1(C)CCC(C)(C)c2cc(Nc3cccc4c5ccccc5n(Cc5ccccc5)c34)c(Br)cc21] | 49 |
| Sub-e7 | 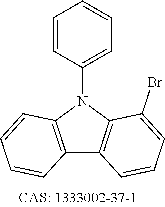 [SMILES: Brc1cccc2c3ccccc3n(-c3ccccc3)c12] | 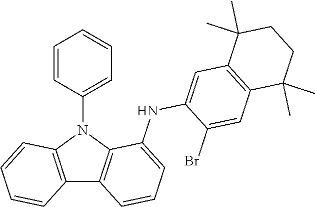 [SMILES: CC1(C)CCC(C)(C)c2cc(Nc3cccc4c5ccccc5n(-c5ccccc5)c34)c(Br)cc21] | 50 |

### Synthesis of Sub-f1

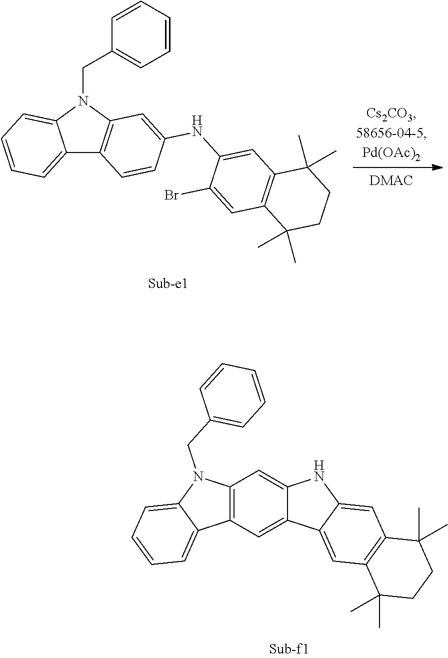 [SMILES: CC1(C)CCC(C)(C)c2cc(Nc3ccc4c5ccccc5n(Cc5ccccc5)c4c3)c(Br)cc21 ; CC1(C)CCC(C)(C)c2cc3c(cc21)[nH]c1cc2c(cc13)c1ccccc1n2Cc1ccccc1]

Under a nitrogen atmosphere, Sub-e1 (26.88 g, 50 mmol), palladium acetate (Pd(OAc)₂, 0.56 g, 2.5 mmol), tricyclohexylphosphine tetrafluoroborate (CAS: 58656-04-5, 1.84 g, 5 mmol), cesium carbonate (Cs₂CO₃, 32.58 g, 100 mmol) and N,N-dimethylacetamide (DMAC, 260 mL) were sequentially added to a 500 mL of three-necked flask, and heated to a refluxing temperature for a reaction under stirring overnight. After being cooled to room temperature, the system was extracted with dichloromethane (100 mL×3 times). The resulting organic phases were combined and dried over anhydrous sodium sulfate, followed by filtration and vacuum distillation to remove the solvent, obtaining a crude product. The crude product was purified by silica column chromatography with n-heptane/dichloromethane as a mobile phase to yield Sub-f1 as a white solid (10.27 g; yield: 45%).

Sub-f2 to Sub-f7 were synthesized respectively with reference to the synthesis of Sub-f1, with Sub-e1 being replaced with Reactant F shown in Table 6.

TABLE 6
Synthesis of Sub-f2 to Sub-f7

| Sub-f | | | Yield |
|---|---|---|---|
| No. | Reactant F | Structure of Sub-f | (%) |
| Sub-f2 | 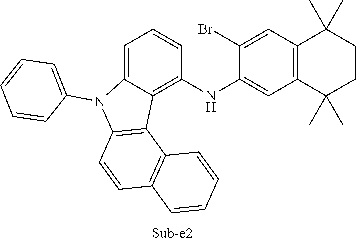 [SMILES: CC1(C)CCC(C)(C)c2cc(Nc3cccc4c3c3c5ccccc5ccc3n4-c3ccccc3)c(Br)cc21] | 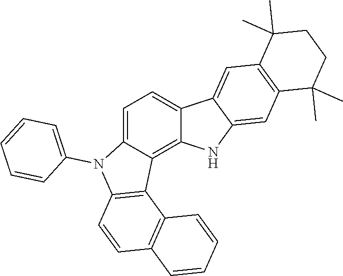 [SMILES: CC1(C)CCC(C)(C)c2cc3c(cc21)[nH]c1c3ccc2c1c1c3ccccc3ccc1n2-c1ccccc1] | 44 |
| Sub-f3 | 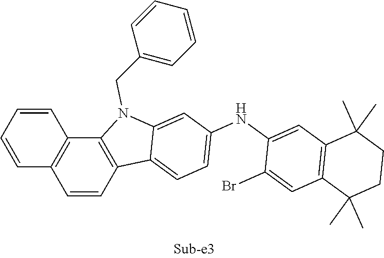 [SMILES: CC1(C)CCC(C)(C)c2cc(Nc3ccc4c5ccc6ccccc6c5n(Cc5ccccc5)c4c3)c(Br)cc21] | 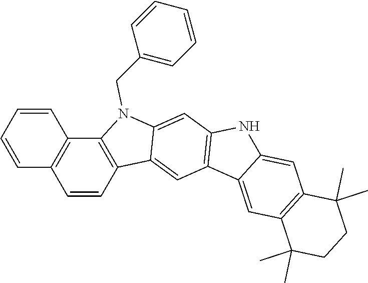 [SMILES: CC1(C)CCC(C)(C)c2cc3c(cc21)[nH]c1cc2c(cc13)c1ccc3ccccc3c1n2Cc1ccccc1] | 45 |
| Sub-f4 | 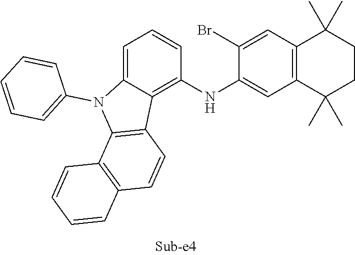 [SMILES: CC1(C)CCC(C)(C)c2cc(Nc3cccc4c3c3ccc5ccccc5c3n4-c3ccccc3)c(Br)cc21] | 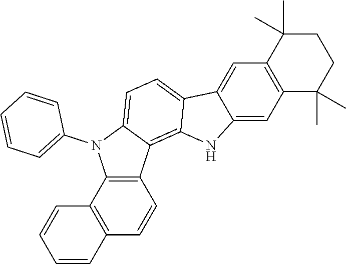 [SMILES: CC1(C)CCC(C)(C)c2cc3c(cc21)[nH]c1c3ccc2c1c1ccc3ccccc3c1n2-c1ccccc1] | 42 |
| Sub-f5 | 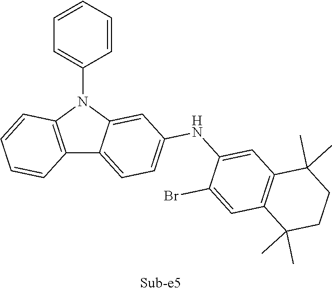 [SMILES: CC1(C)CCC(C)(C)c2cc(Nc3ccc4c5ccccc5n(-c5ccccc5)c4c3)c(Br)cc21] | 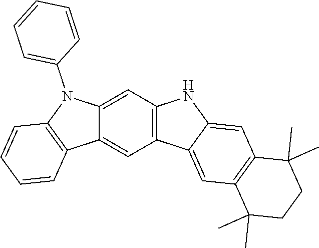 [SMILES: CC1(C)CCC(C)(C)c2cc3c(cc21)[nH]c1cc2c(cc13)c1ccccc1n2-c1ccccc1] | 41 |
| Sub-f6 | 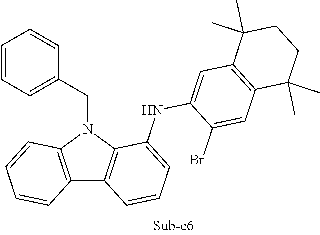 [SMILES: CC1(C)CCC(C)(C)c2cc(Nc3cccc4c5ccccc5n(Cc5ccccc5)c34)c(Br)cc21] | 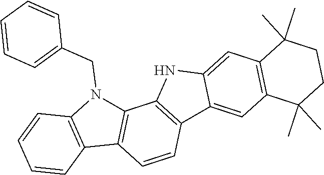 [SMILES: CC1(C)CCC(C)(C)c2cc3c(cc21)[nH]c1c3ccc2c3ccccc3n(Cc3ccccc3)c21] | 44 |
| Sub-f7 | 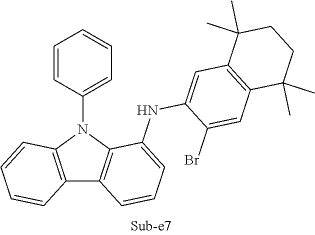 [SMILES: CC1(C)CCC(C)(C)c2cc(Nc3cccc4c5ccccc5n(-c5ccccc5)c34)c(Br)cc21] | 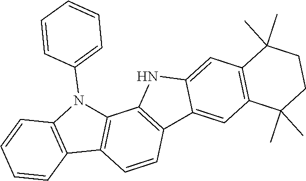 [SMILES: CC1(C)CCC(C)(C)c2cc3c(cc21)[nH]c1c3ccc2c3ccccc3n(-c3ccccc3)c21] | 40 |

### Synthesis of Sub-g1

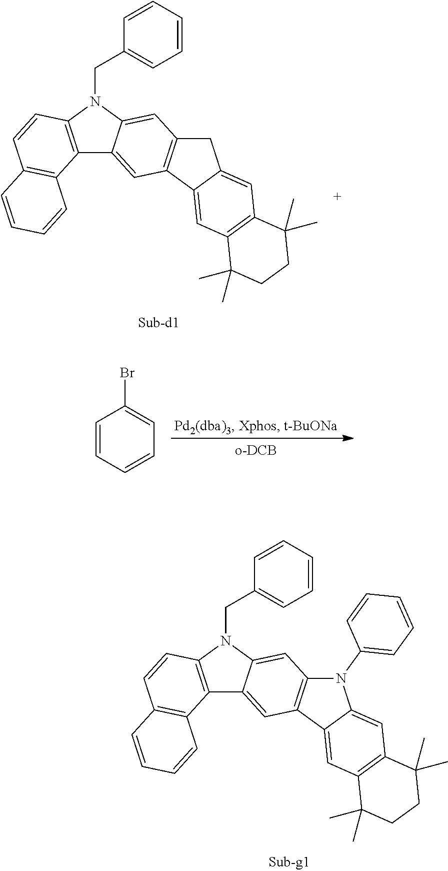

Under a nitrogen atmosphere, Sub-d1 (25.33 g, 50 mmol), bromobenzene (7.85 g, 55 mmol), tris(dibenzylideneacetone)dipalladium (Pd₂(dba)₂, 0.916 g, 1 mmol), 2-dicyclohexylphosphino-2′,4′,6′-triisopropylbiphenyl (XPhos, 0.95 g, 2 mmol), sodium tert-butanoate (t-BuONa, 9.61 g, 100 mmol) and xylene (250 mL) were sequentially added to a 500 mL of three-necked flask, and heated to a refluxing temperature for a reaction under stirring overnight. After being cooled to room temperature, the system was extracted with dichloromethane (100 mL×3 times). The resulting organic phases were combined and dried over anhydrous sodium sulfate, followed by filtration and vacuum distillation to remove the solvent, obtaining a crude product. The crude product was purified by silica column chromatography with n-heptane/dichloromethane as a mobile phase to yield sub-g1 as a white solid (15.05 g, yield: 73%).

Sub-g2 to Sub-g13 were synthesized respectively with reference to the synthesis of Sub-g1, with Sub-d1 being replaced with Reactant G shown in Table 7.

TABLE 7
Synthesis of Sub-g2 to Sub-g13

| Sub-g | | | Yield |
|---|---|---|---|
| No. | Reactant G | Structure of Sub-g | (%) |
| Sub-g2 | 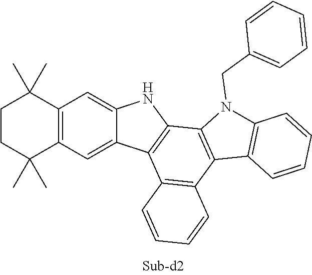 [SMILES: CC1(C)CCC(C)(C)c2cc3c(cc21)[nH]c1c3c2ccccc2c2c3ccccc3n(Cc3ccccc3)c12] | 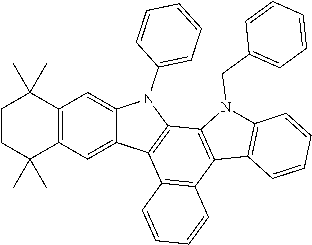 [SMILES: CC1(C)CCC(C)(C)c2cc3c(cc21)c1c2ccccc2c2c4ccccc4n(Cc4ccccc4)c2c1n3-c1ccccc1] | 67 |
| Sub-g3 | 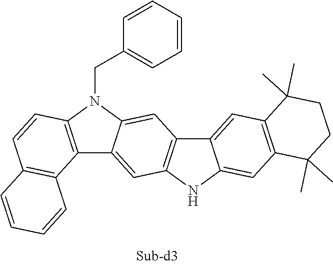 [SMILES: CC1(C)CCC(C)(C)c2cc3c(cc21)[nH]c1cc2c4c5ccccc5ccc4n(Cc4ccccc4)c2cc13] | 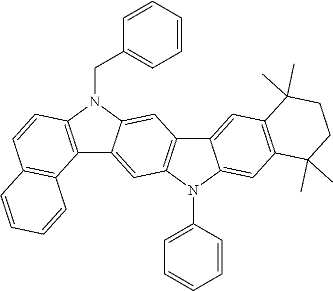 [SMILES: CC1(C)CCC(C)(C)c2cc3c(cc21)c1cc2c(cc1n3-c1ccccc1)c1c3ccccc3ccc1n2Cc1ccccc1] | 68 |
| Sub-g4 | 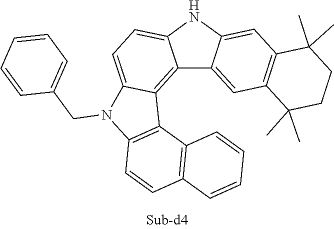 [SMILES: CC1(C)CCC(C)(C)c2cc3c(cc21)[nH]c1ccc2c(c13)c1c3ccccc3ccc1n2Cc1ccccc1] | 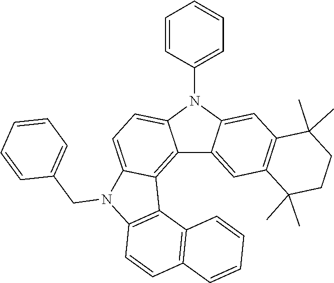 [SMILES: CC1(C)CCC(C)(C)c2cc3c(cc21)c1c2c4c5ccccc5ccc4n(Cc4ccccc4)c2ccc1n3-c1ccccc1] | 71 |
| Sub-g5 | 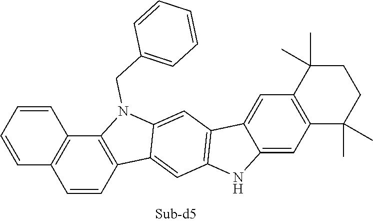 [SMILES: CC1(C)CCC(C)(C)c2cc3c(cc21)[nH]c1cc2c4ccc5ccccc5c4n(Cc4ccccc4)c2cc13] | 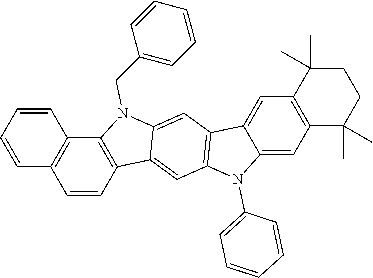 [SMILES: CC1(C)CCC(C)(C)c2cc3c(cc21)c1cc2c(cc1n3-c1ccccc1)c1ccc3ccccc3c1n2Cc1ccccc1] | 68 |
| Sub-g6 | 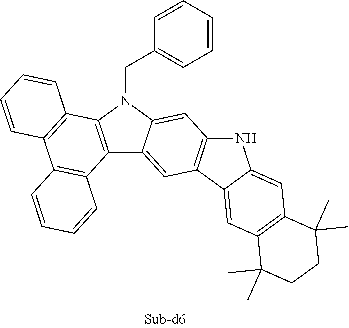 [SMILES: CC1(C)CCC(C)(C)c2cc3c(cc21)[nH]c1cc2c(cc13)c1c3ccccc3c3ccccc3c1n2Cc1ccccc1] | 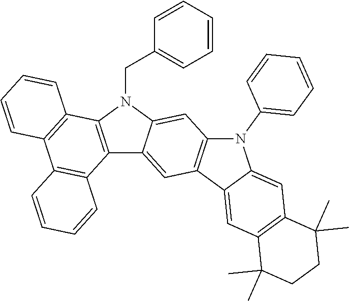 [SMILES: CC1(C)CCC(C)(C)c2cc3c(cc21)c1cc2c4c5ccccc5c5ccccc5c4n(Cc4ccccc4)c2cc1n3-c1ccccc1] | 65 |
| Sub-g7 | 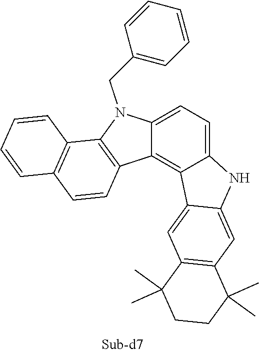 [SMILES: CC1(C)CCC(C)(C)c2cc3c(cc21)[nH]c1ccc2c(c4ccc5ccccc5c4n2Cc2ccccc2)c13] | 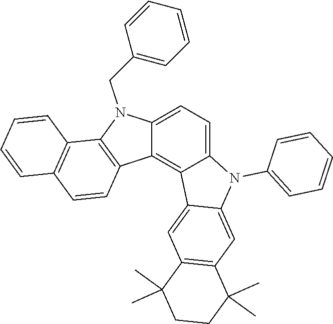 [SMILES: CC1(C)CCC(C)(C)c2cc3c(cc21)c1c2c4ccc5ccccc5c4n(Cc4ccccc4)c2ccc1n3-c1ccccc1] | 68 |
| Sub-g8 | 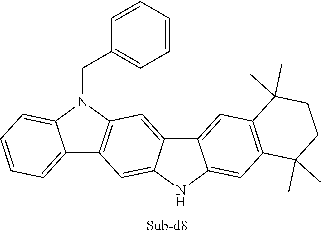 [SMILES: CC1(C)CCC(C)(C)c2cc3c(cc21)[nH]c1cc2c4ccccc4n(Cc4ccccc4)c2cc13] | 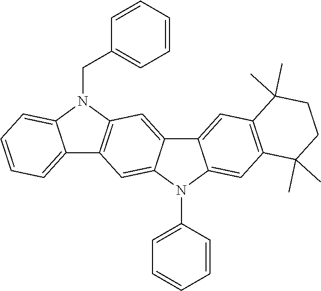 [SMILES: CC1(C)CCC(C)(C)c2cc3c(cc21)c1cc2c(cc1n3-c1ccccc1)c1ccccc1n2Cc1ccccc1] | 65 |
| Sub-g9 | 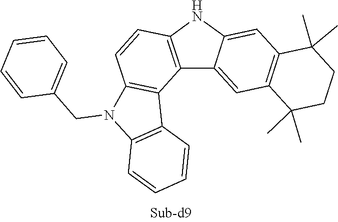 [SMILES: CC1(C)CCC(C)(C)c2cc3c(cc21)[nH]c1ccc2c(c4ccccc4n2Cc2ccccc2)c13] | 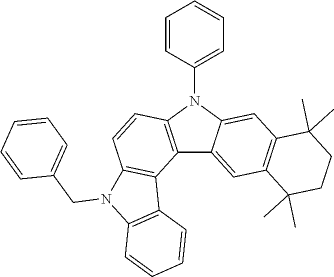 [SMILES: CC1(C)CCC(C)(C)c2cc3c(cc21)c1c2c4ccccc4n(Cc4ccccc4)c2ccc1n3-c1ccccc1] | 72 |
| Sub-g10 | 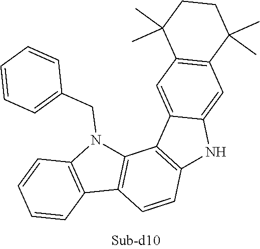 [SMILES: CC1(C)CCC(C)(C)c2cc3c(cc21)[nH]c1ccc2c4ccccc4n(Cc4ccccc4)c2c13] | 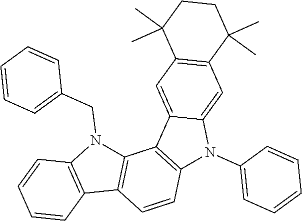 [SMILES: CC1(C)CCC(C)(C)c2cc3c(cc21)c1c2c(ccc1n3-c1ccccc1)c1ccccc1n2Cc1ccccc1] | 74 |
| Sub-g11 | 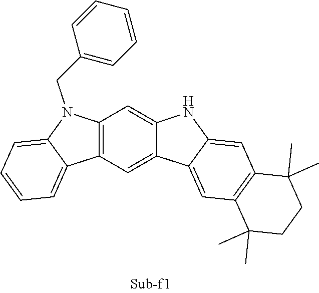 [SMILES: CC1(C)CCC(C)(C)c2cc3c(cc21)[nH]c1cc2c(cc13)c1ccccc1n2Cc1ccccc1] | 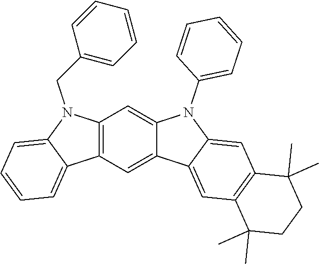 [SMILES: CC1(C)CCC(C)(C)c2cc3c(cc21)c1cc2c4ccccc4n(Cc4ccccc4)c2cc1n3-c1ccccc1] | 66 |
| Sub-g12 | 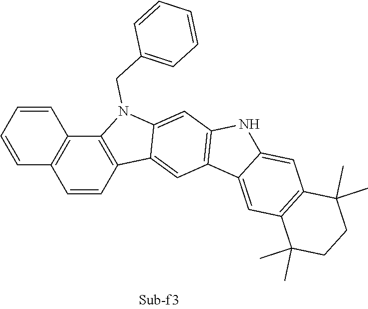 [SMILES: CC1(C)CCC(C)(C)c2cc3c(cc21)[nH]c1cc2c(cc13)c1ccc3ccccc3c1n2Cc1ccccc1] | 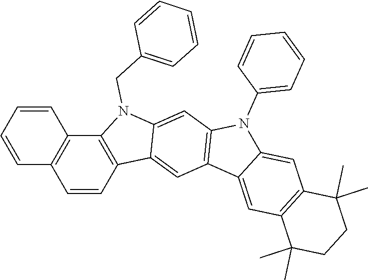 [SMILES: CC1(C)CCC(C)(C)c2cc3c(cc21)c1cc2c4ccc5ccccc5c4n(Cc4ccccc4)c2cc1n3-c1ccccc1] | 73 |
| Sub-g13 | 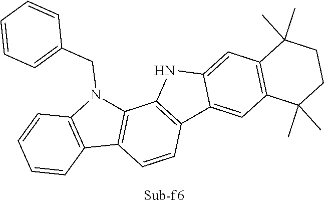 [SMILES: CC1(C)CCC(C)(C)c2cc3c(cc21)[nH]c1c3ccc2c3ccccc3n(Cc3ccccc3)c21] | 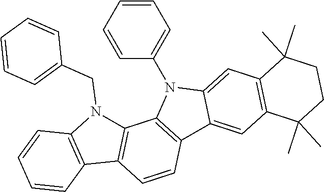 [SMILES: CC1(C)CCC(C)(C)c2cc3c(cc21)c1ccc2c4ccccc4n(Cc4ccccc4)c2c1n3-c1ccccc1] | 72 |

### Synthesis of Sub-g14

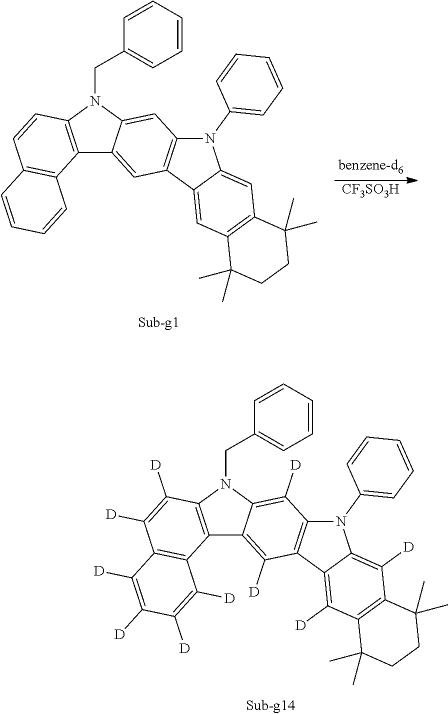

Sub-g1 (14.57 g, 25 mmol) and 200 mL of benzene-d₆ were added sequentially to a 100 mL of three-necked flask under a nitrogen atmosphere, and heated to 60° C., followed by adding trifluoromethanesulfonic acid (CF₃SO₃H, 22.51 g, 150 mmol), and continued heating to boiling for a reaction under stirring for 24 hours. After the reaction system was cooled to room temperature, 50 mL of heavy water was added thereto, followed by stirring for 10 minutes, and then adding a saturated aqueous solution of K₃PO₄ to neutralize the reaction solution. The resulting organic layers (50 mL×3 times) were extracted with dichloromethane, and the resulting organic phases were combined and dried over anhydrous sodium sulfate, followed by filtration and vacuum distillation to remove the solvent, obtaining a crude product. The crude product was purified by silica column chromatography with n-heptane/dichloromethane as a mobile phase to yield Sub-g14 as a white solid (9.48 g, yield: 64%).

Sub-g15 was synthesized with reference to the synthesis of Sub-g14, with Sub-g1 being replaced with Reactant H shown in Table 8.

TABLE 8
Synthesis of Sub-g15

| Sub-g No. | Reactant H | Structure of Sub-g | Yield (%) |
|---|---|---|---|
| Sub-g15 | 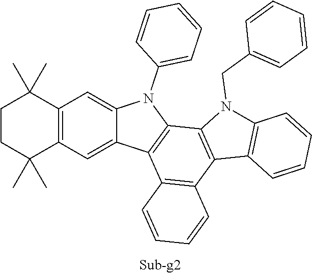 [SMILES: CC1(C)CCC(C)(C)c2cc3c(cc21)c1c2ccccc2c2c4ccccc4n(Cc4ccccc4)c2c1n3-c1ccccc1] | 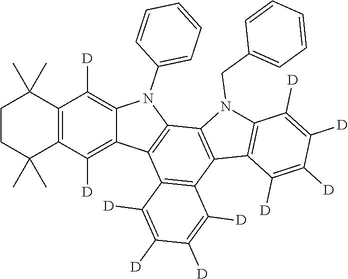 [SMILES: [2H]c1c([2H])c([2H])c2c(c1[2H])c1c3c([2H])c([2H])c([2H])c([2H])c3n(Cc3ccccc3)c1c1c2c2c([2H])c3c(c([2H])c2n1-c1ccccc1)C(C)(C)CCC3(C)C] | 61 |

### Synthesis of Sub-h1

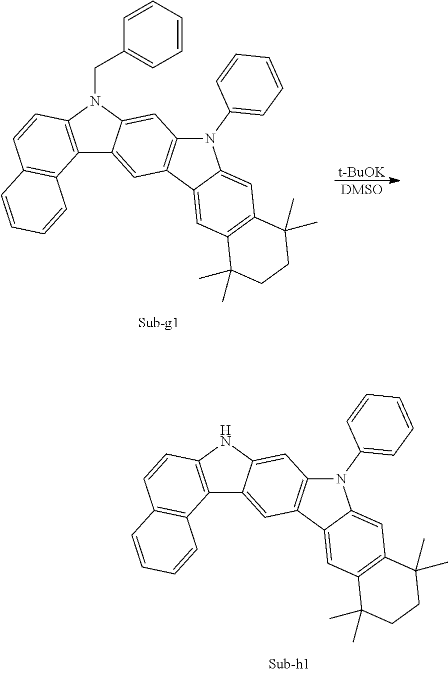

Under a nitrogen atmosphere, Sub-g1 (20.11 g, 50 mmol), potassium tert-butoxide (t-BuOK, 56.10 g, 500 mmol) and DMSO (dimethyl sulfoxide, 300 mL) were sequentially added to a 500 mL of three-necked flask, stirred and heated to 60° C. for a reaction for 4 hours. After the system was cooled to room temperature, the reaction solution was poured into 500 mL of deionized water, and a precipitate was formed. The precipitate was filtered and the resulting filter cake was collected. The filter cake was dissolved in dichloromethane (200 mL), then dried by adding anhydrous sodium sulfate, and filtered. The resulting filtrate was collected and subjected to vacuum distillation to remove the solvent, obtaining a crude product. The crude product was purified by silica column chromatography with n-heptane/dichloromethane as a mobile phase to yield Sub-h1 as a white solid (13.11 g, yield: 84%).

Sub-h2 to Sub-h15 were synthesized with reference to following the synthesis of Sub-h1, with Sub-g1 being replaced with Reactant J shown in Table 9.

TABLE 9
Synthesis of Sub-h2 to Sub-h15

| Sub-h | | | Yield |
|---|---|---|---|
| No. | Reactant J | Structure of Sub-h | (%) |
| Sub-h2 | 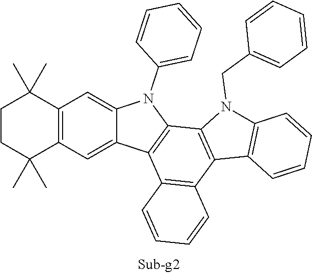 [SMILES: CC1(C)CCC(C)(C)c2cc3c(cc21)c1c2ccccc2c2c4ccccc4n(Cc4ccccc4)c2c1n3-c1ccccc1] | 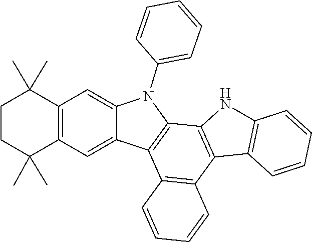 [SMILES: CC1(C)CCC(C)(C)c2cc3c(cc21)c1c2ccccc2c2c4ccccc4[nH]c2c1n3-c1ccccc1] | 83 |
| Sub-h3 | 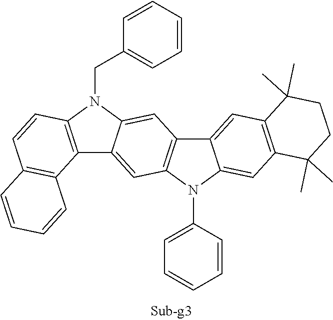 [SMILES: CC1(C)CCC(C)(C)c2cc3c(cc21)c1cc2c(cc1n3-c1ccccc1)c1c3ccccc3ccc1n2Cc1ccccc1] | 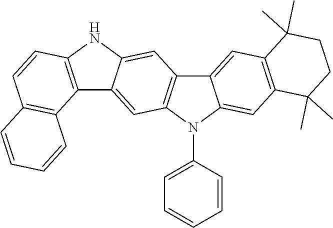 [SMILES: CC1(C)CCC(C)(C)c2cc3c(cc21)c1cc2[nH]c4ccc5ccccc5c4c2cc1n3-c1ccccc1] | 80 |
| Sub-h4 | 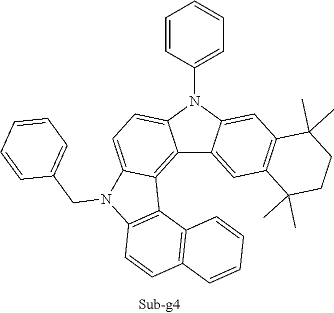 [SMILES: CC1(C)CCC(C)(C)c2cc3c(cc21)c1c2c4c5ccccc5ccc4n(Cc4ccccc4)c2ccc1n3-c1ccccc1] | 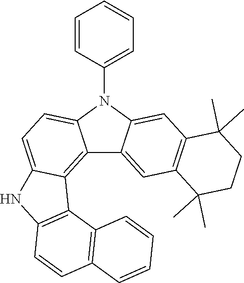 [SMILES: CC1(C)CCC(C)(C)c2cc3c(cc21)c1c2c(ccc1n3-c1ccccc1)[nH]c1ccc3ccccc3c12] | 81 |
| Sub-h5 | 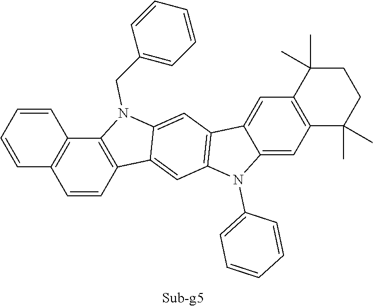 [SMILES: CC1(C)CCC(C)(C)c2cc3c(cc21)c1cc2c(cc1n3-c1ccccc1)c1ccc3ccccc3c1n2Cc1ccccc1] | 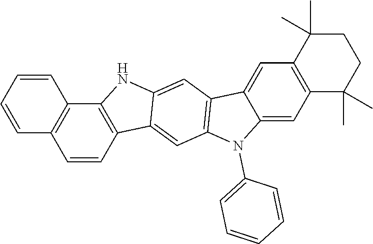 [SMILES: CC1(C)CCC(C)(C)c2cc3c(cc21)c1cc2[nH]c4c5ccccc5ccc4c2cc1n3-c1ccccc1] | 81 |
| Sub-h6 | 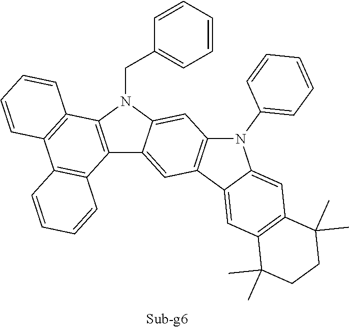 [SMILES: CC1(C)CCC(C)(C)c2cc3c(cc21)c1cc2c4c5ccccc5c5ccccc5c4n(Cc4ccccc4)c2cc1n3-c1ccccc1] | 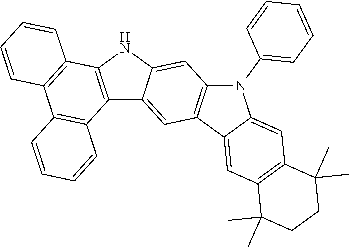 [SMILES: CC1(C)CCC(C)(C)c2cc3c(cc21)c1cc2c(cc1n3-c1ccccc1)[nH]c1c3ccccc3c3ccccc3c21] | 81 |
| Sub-h7 | 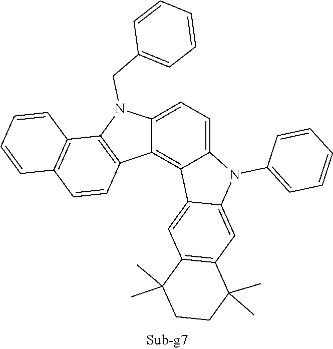 [SMILES: CC1(C)CCC(C)(C)c2cc3c(cc21)c1c2c4ccc5ccccc5c4n(Cc4ccccc4)c2ccc1n3-c1ccccc1] | 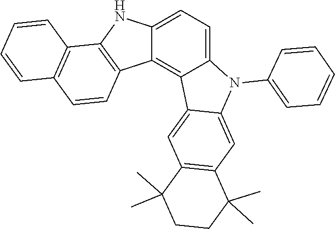 [SMILES: CC1(C)CCC(C)(C)c2cc3c(cc21)c1c2c(ccc1n3-c1ccccc1)[nH]c1c3ccccc3ccc12] | 83 |
| Sub-h8 | 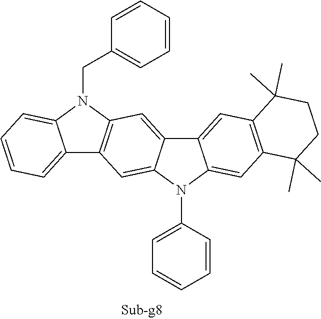 [SMILES: CC1(C)CCC(C)(C)c2cc3c(cc21)c1cc2c(cc1n3-c1ccccc1)c1ccccc1n2Cc1ccccc1] | 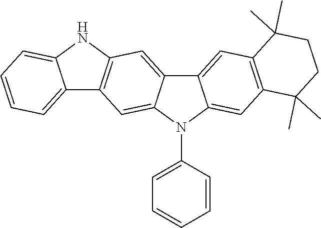 [SMILES: CC1(C)CCC(C)(C)c2cc3c(cc21)c1cc2[nH]c4ccccc4c2cc1n3-c1ccccc1] | 82 |
| Sub-h9 | 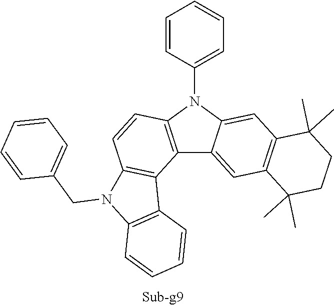 [SMILES: CC1(C)CCC(C)(C)c2cc3c(cc21)c1c2c4ccccc4n(Cc4ccccc4)c2ccc1n3-c1ccccc1] | 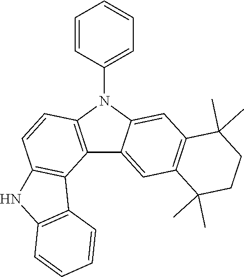 [SMILES: CC1(C)CCC(C)(C)c2cc3c(cc21)c1c2c(ccc1n3-c1ccccc1)[nH]c1ccccc12] | 80 |
| Sub-h10 | 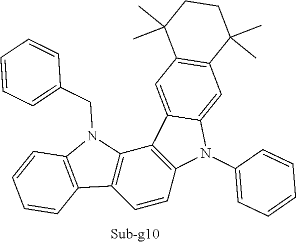 [SMILES: CC1(C)CCC(C)(C)c2cc3c(cc21)c1c2c(ccc1n3-c1ccccc1)c1ccccc1n2Cc1ccccc1] | 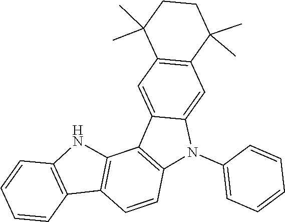 [SMILES: CC1(C)CCC(C)(C)c2cc3c(cc21)c1c2[nH]c4ccccc4c2ccc1n3-c1ccccc1] | 82 |
| Sub-h11 | 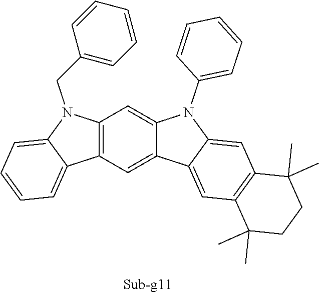 [SMILES: CC1(C)CCC(C)(C)c2cc3c(cc21)c1cc2c4ccccc4n(Cc4ccccc4)c2cc1n3-c1ccccc1] | 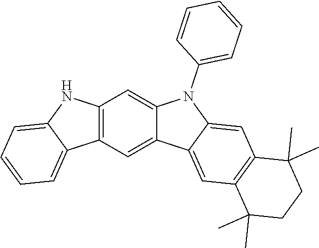 [SMILES: CC1(C)CCC(C)(C)c2cc3c(cc21)c1cc2c(cc1n3-c1ccccc1)[nH]c1ccccc12] | 84 |
| Sub-h12 | 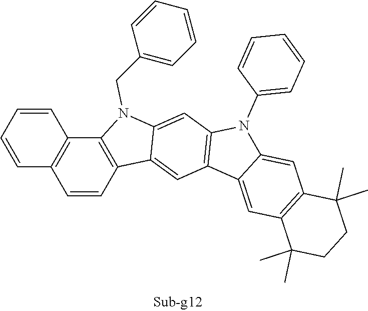 [SMILES: CC1(C)CCC(C)(C)c2cc3c(cc21)c1cc2c4ccc5ccccc5c4n(Cc4ccccc4)c2cc1n3-c1ccccc1] | 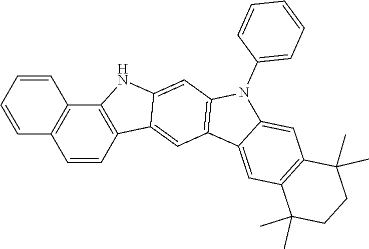 [SMILES: CC1(C)CCC(C)(C)c2cc3c(cc21)c1cc2c(cc1n3-c1ccccc1)[nH]c1c3ccccc3ccc21] | 80 |
| Sub-h13 | 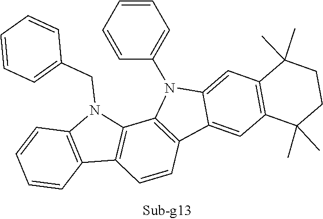 [SMILES: CC1(C)CCC(C)(C)c2cc3c(cc21)c1ccc2c4ccccc4n(Cc4ccccc4)c2c1n3-c1ccccc1] | 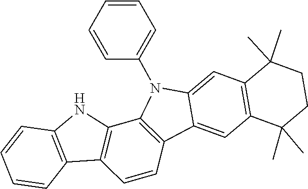 [SMILES: CC1(C)CCC(C)(C)c2cc3c(cc21)c1ccc2c4ccccc4[nH]c2c1n3-c1ccccc1] | 83 |
| Sub-h14 | 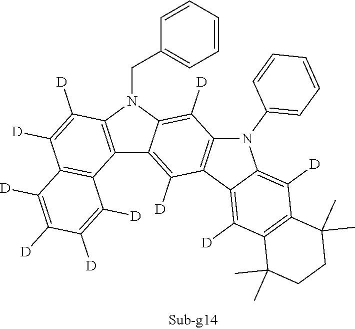 [SMILES: [2H]c1c([2H])c([2H])c2c(c1[2H])c([2H])c([2H])c1c2c2c([2H])c3c4c([2H])c5c(c([2H])c4n(-c4ccccc4)c3c([2H])c2n1Cc1ccccc1)C(C)(C)CCC5(C)C] | 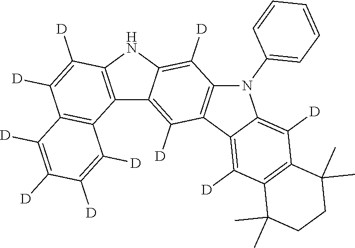 [SMILES: [2H]c1c([2H])c([2H])c2c(c1[2H])c([2H])c([2H])c1[nH]c3c([2H])c4c(c([2H])c3c12)c1c([2H])c2c(c([2H])c1n4-c1ccccc1)C(C)(C)CCC2(C)C] | 82 |
| Sub-h15 | 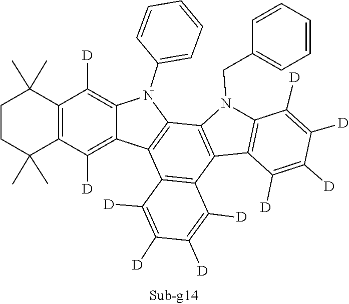 [SMILES: [2H]c1c([2H])c([2H])c2c(c1[2H])c1c3c([2H])c([2H])c([2H])c([2H])c3n(Cc3ccccc3)c1c1c2c2c([2H])c3c(c([2H])c2n1-c1ccccc1)C(C)(C)CCC3(C)C] | 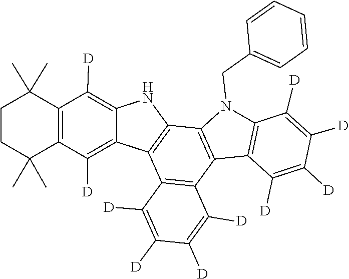 [SMILES: [2H]c1c([2H])c([2H])c2c(c1[2H])c1c([nH]c3c([2H])c4c(c([2H])c31)C(C)(C)CCC4(C)C)c1c2c2c([2H])c([2H])c([2H])c([2H])c2n1Cc1ccccc1] | 82 |

### Synthesis of Compound A-3

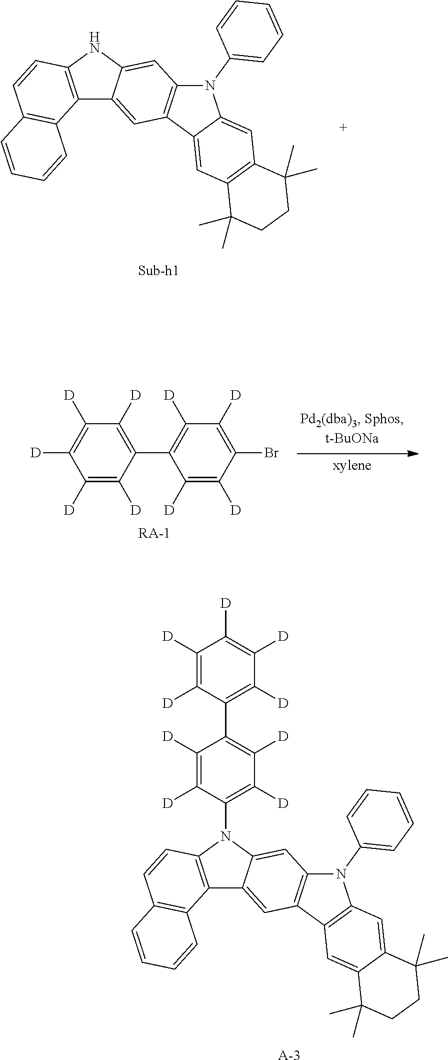

Under a nitrogen atmosphere, Sub-h1 (12.32 g, 25 mmol), RA-1 (CAS: 142475-00-1, 7.26 g, mmol), tris(dibenzylideneacetone)dipalladium (Pd₂(dba)₃, 0.46 g, 0.5 mmol), 2-dicyclohexylphosphino-2′,4′,6′-triisopropylbiphenyl (SPhos, 0.41 g, 1 mmol), sodium tert-butoxide (t-BuONa, 4.80 g, 50 mmol), and xylene (120 ML) were added sequentially to a 250 mL of three-necked flask, heated to a refluxing temperature for a reaction under stirring overnight. After being cooled to room temperature, the system was extracted with dichloromethane (100 mL×3 times). The resulting organic phases were combined and dried over anhydrous sodium sulfate, followed by filtration and vacuum distillation to remove the solvent, obtaining a crude product. The crude product was purified by silica column chromatography with n-heptane/dichloromethane as a mobile phase to yield Compound A-3 as a white solid (10.29 g; yield: 63%), m/z=654.4 [M+H]⁺.

The following compounds of the present application were synthesized respectively with reference to the synthesis of Compound A-3, with Sub-h1 being replaced with Reactant K shown in Table 10 and RA-1 being replaced with Reactant L shown in Table 10.

TABLE 10
Synthesis of compounds of the present application

| | | Structure and No. of | m/z | Yield |
|---|---|---|---|---|
| Reactant K | Reactant L | Compound | ([M + H]⁺) | (%) |
| 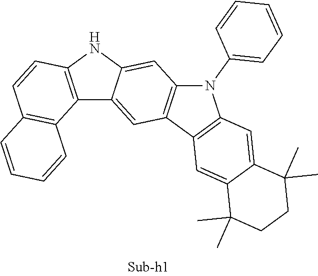 [SMILES: CC1(C)CCC(C)(C)c2cc3c(cc21)c1cc2c(cc1n3-c1ccccc1)[nH]c1ccc3ccccc3c12] | 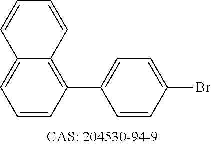 [SMILES: Brc1ccc(-c2cccc3ccccc23)cc1] | 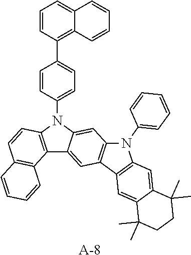 [SMILES: CC1(C)CCC(C)(C)c2cc3c(cc21)c1cc2c4c5ccccc5ccc4n(-c4ccc(-c5cccc6ccccc56)cc4)c2cc1n3-c1ccccc1 ; "A-8"] | 695.3 | 65 |
| 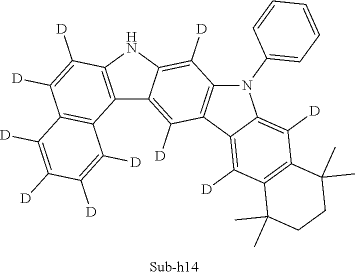 [SMILES: [2H]c1c([2H])c([2H])c2c(c1[2H])c([2H])c([2H])c1[nH]c3c([2H])c4c(c([2H])c3c12)c1c([2H])c2c(c([2H])c1n4-c1ccccc1)C(C)(C)CCC2(C)C] | 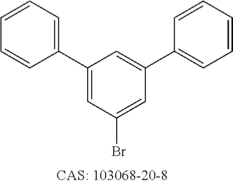 [SMILES: Brc1cc(-c2ccccc2)cc(-c2ccccc2)c1] | 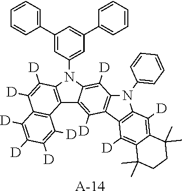 [SMILES: [2H]c1c([2H])c([2H])c2c(c1[2H])c([2H])c([2H])c1c2c2c([2H])c3c4c([2H])c5c(c([2H])c4n(-c4ccccc4)c3c([2H])c2n1-c1cc(-c2ccccc2)cc(-c2ccccc2)c1)C(C)(C)CCC5(C)C ; "A-14"] | 731.4 | 54 |
| 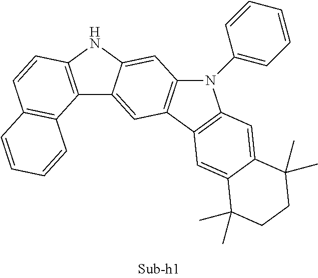 [SMILES: CC1(C)CCC(C)(C)c2cc3c(cc21)c1cc2c(cc1n3-c1ccccc1)[nH]c1ccc3ccccc3c12] | 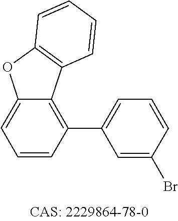 [SMILES: Brc1cccc(-c2cccc3oc4ccccc4c23)c1] | 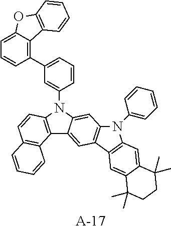 [SMILES: CC1(C)CCC(C)(C)c2cc3c(cc21)c1cc2c4c5ccccc5ccc4n(-c4cccc(-c5cccc6oc7ccccc7c56)c4)c2cc1n3-c1ccccc1 ; "A-17"] | 735.3 | 55 |
| 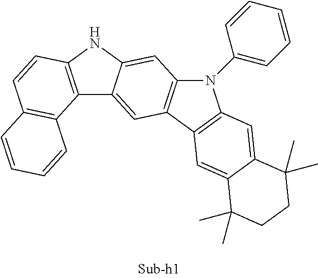 [SMILES: CC1(C)CCC(C)(C)c2cc3c(cc21)c1cc2c(cc1n3-c1ccccc1)[nH]c1ccc3ccccc3c12] | 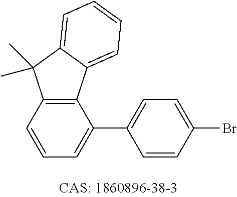 [SMILES: CC1(C)c2ccccc2-c2c(-c3ccc(Br)cc3)cccc21] | 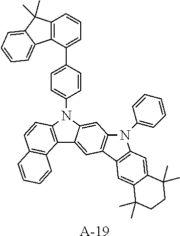 [SMILES: CC1(C)CCC(C)(C)c2cc3c(cc21)c1cc2c4c5ccccc5ccc4n(-c4ccc(-c5cccc6c5-c5ccccc5C6(C)C)cc4)c2cc1n3-c1ccccc1 ; "A-19"] | 761.4 | 60 |
| 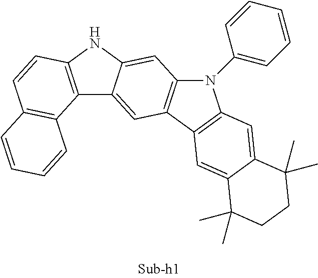 [SMILES: CC1(C)CCC(C)(C)c2cc3c(cc21)c1cc2c(cc1n3-c1ccccc1)[nH]c1ccc3ccccc3c12] | 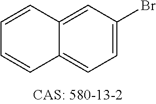 [SMILES: Brc1ccc2ccccc2c1] | 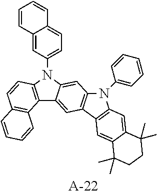 [SMILES: CC1(C)CCC(C)(C)c2cc3c(cc21)c1cc2c4c5ccccc5ccc4n(-c4ccc5ccccc5c4)c2cc1n3-c1ccccc1 ; "A-22"] | 619.3 | 52 |
| 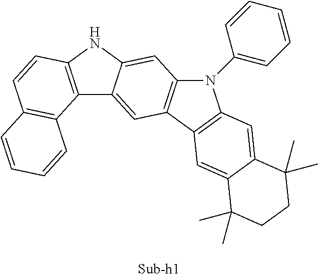 [SMILES: CC1(C)CCC(C)(C)c2cc3c(cc21)c1cc2c(cc1n3-c1ccccc1)[nH]c1ccc3ccccc3c12] | 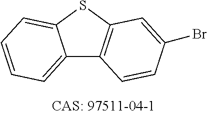 [SMILES: Brc1ccc2c(c1)sc1ccccc12] | 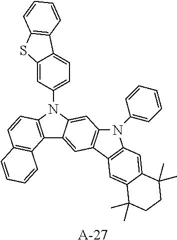 [SMILES: CC1(C)CCC(C)(C)c2cc3c(cc21)c1cc2c4c5ccccc5ccc4n(-c4ccc5c(c4)sc4ccccc45)c2cc1n3-c1ccccc1 ; "A-27"] | 675.3 | 52 |
| 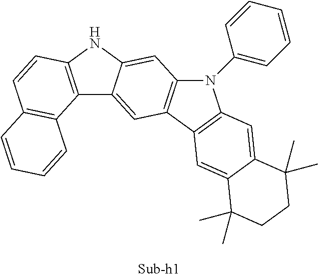 [SMILES: CC1(C)CCC(C)(C)c2cc3c(cc21)c1cc2c(cc1n3-c1ccccc1)[nH]c1ccc3ccccc3c12] | 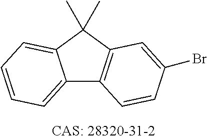 [SMILES: CC1(C)c2ccccc2-c2ccc(Br)cc21] | 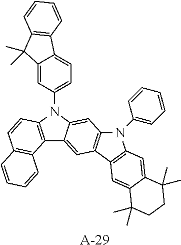 [SMILES: CC1(C)CCC(C)(C)c2cc3c(cc21)c1cc2c4c5ccccc5ccc4n(-c4ccc5c(c4)C(C)(C)c4ccccc4-5)c2cc1n3-c1ccccc1 ; "A-29"] | 685.4 | 54 |
| 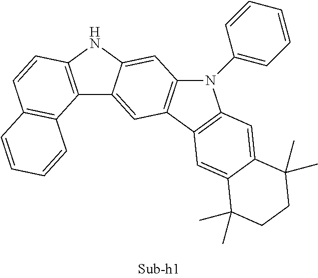 [SMILES: CC1(C)CCC(C)(C)c2cc3c(cc21)c1cc2c(cc1n3-c1ccccc1)[nH]c1ccc3ccccc3c12] | 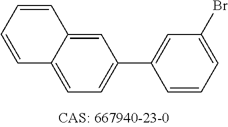 [SMILES: Brc1cccc(-c2ccc3ccccc3c2)c1] | 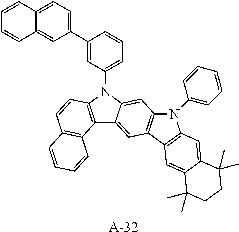 [SMILES: CC1(C)CCC(C)(C)c2cc3c(cc21)c1cc2c4c5ccccc5ccc4n(-c4cccc(-c5ccc6ccccc6c5)c4)c2cc1n3-c1ccccc1 ; "A-32"] | 695.3 | 63 |
| 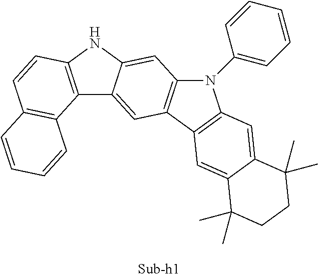 [SMILES: CC1(C)CCC(C)(C)c2cc3c(cc21)c1cc2c(cc1n3-c1ccccc1)[nH]c1ccc3ccccc3c12] | 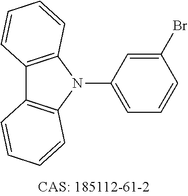 [SMILES: Brc1cccc(-n2c3ccccc3c3ccccc32)c1] | 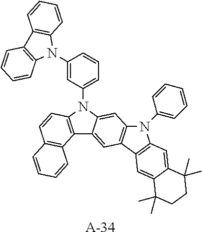 [SMILES: CC1(C)CCC(C)(C)c2cc3c(cc21)c1cc2c4c5ccccc5ccc4n(-c4cccc(-n5c6ccccc6c6ccccc65)c4)c2cc1n3-c1ccccc1 ; "A-34"] | 734.4 | 52 |
| 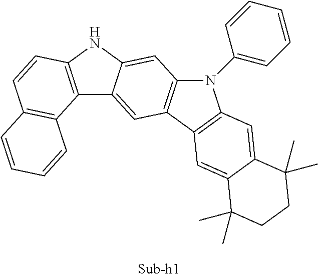 [SMILES: CC1(C)CCC(C)(C)c2cc3c(cc21)c1cc2c(cc1n3-c1ccccc1)[nH]c1ccc3ccccc3c12] | 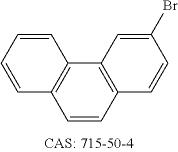 [SMILES: Brc1ccc2ccc3ccccc3c2c1] | 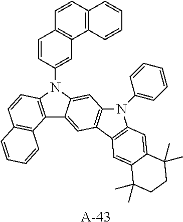 [SMILES: CC1(C)CCC(C)(C)c2cc3c(cc21)c1cc2c4c5ccccc5ccc4n(-c4ccc5ccc6ccccc6c5c4)c2cc1n3-c1ccccc1 ; "A-43"] | 669.3 | 64 |
| 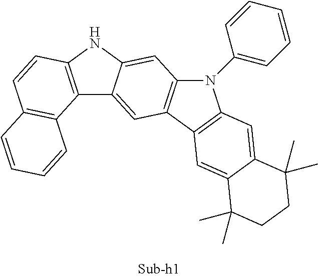 [SMILES: CC1(C)CCC(C)(C)c2cc3c(cc21)c1cc2c(cc1n3-c1ccccc1)[nH]c1ccc3ccccc3c12] | 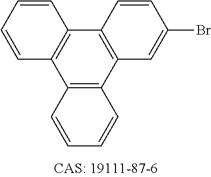 [SMILES: Brc1ccc2c3ccccc3c3ccccc3c2c1] | 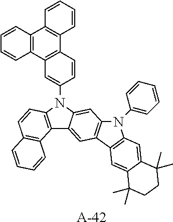 [SMILES: CC1(C)CCC(C)(C)c2cc3c(cc21)c1cc2c4c5ccccc5ccc4n(-c4ccc5c6ccccc6c6ccccc6c5c4)c2cc1n3-c1ccccc1 ; "A-42"] | 719.3 | 57 |
| 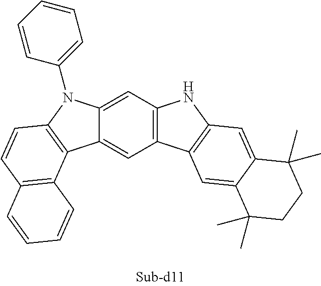 [SMILES: CC1(C)CCC(C)(C)c2cc3c(cc21)[nH]c1cc2c(cc13)c1c3ccccc3ccc1n2-c1ccccc1] | 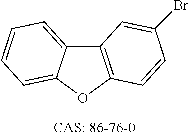 [SMILES: Brc1ccc2oc3ccccc3c2c1] | 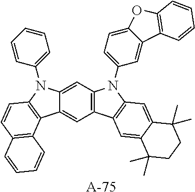 [SMILES: CC1(C)CCC(C)(C)c2cc3c(cc21)c1cc2c4c5ccccc5ccc4n(-c4ccccc4)c2cc1n3-c1ccc2oc3ccccc3c2c1 ; "A-75"] | 659.3 | 54 |
| 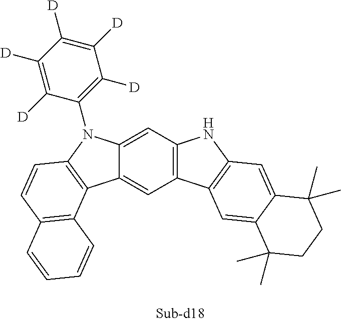 [SMILES: [2H]c1c([2H])c([2H])c(-n2c3cc4[nH]c5cc6c(cc5c4cc3c3c4ccccc4ccc32)C(C)(C)CCC6(C)C)c([2H])c1[2H]] | 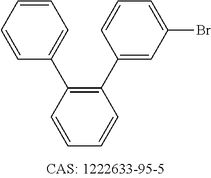 [SMILES: Brc1cccc(-c2ccccc2-c2ccccc2)c1] | 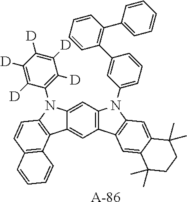 [SMILES: [2H]c1c([2H])c([2H])c(-n2c3cc4c(cc3c3c5ccccc5ccc32)c2cc3c(cc2n4-c2cccc(-c4ccccc4-c4ccccc4)c2)C(C)(C)CCC3(C)C)c([2H])c1[2H] ; "A-86"] | 726.4 | 51 |
| 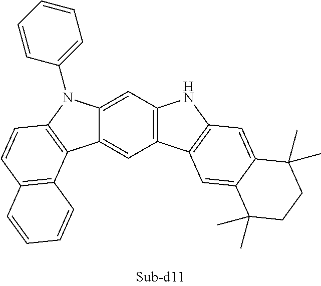 [SMILES: CC1(C)CCC(C)(C)c2cc3c(cc21)[nH]c1cc2c(cc13)c1c3ccccc3ccc1n2-c1ccccc1] | 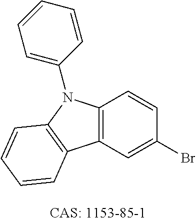 [SMILES: Brc1ccc2c(c1)c1ccccc1n2-c1ccccc1] | 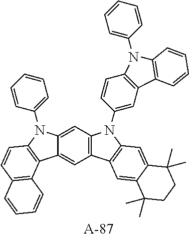 [SMILES: CC1(C)CCC(C)(C)c2cc3c(cc21)c1cc2c4c5ccccc5ccc4n(-c4ccccc4)c2cc1n3-c1ccc2c(c1)c1ccccc1n2-c1ccccc1 ; "A-87"] | 734.4 | 63 |
| 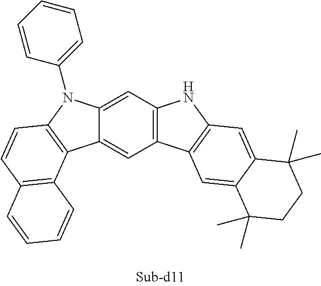 [SMILES: CC1(C)CCC(C)(C)c2cc3c(cc21)[nH]c1cc2c(cc13)c1c3ccccc3ccc1n2-c1ccccc1] | 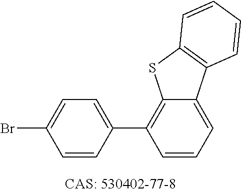 [SMILES: Brc1ccc(-c2cccc3c2sc2ccccc23)cc1] | 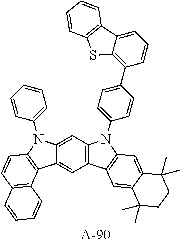 [SMILES: CC1(C)CCC(C)(C)c2cc3c(cc21)c1cc2c4c5ccccc5ccc4n(-c4ccccc4)c2cc1n3-c1ccc(-c2cccc3c2sc2ccccc23)cc1 ; "A-90"] | 751.3 | 65 |
| 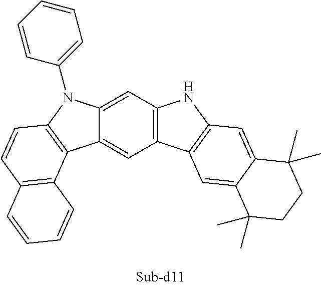 [SMILES: CC1(C)CCC(C)(C)c2cc3c(cc21)[nH]c1cc2c(cc13)c1c3ccccc3ccc1n2-c1ccccc1] | 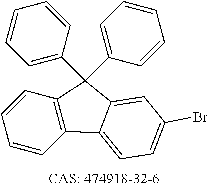 [SMILES: Brc1ccc2c(c1)C(c1ccccc1)(c1ccccc1)c1ccccc1-2] | 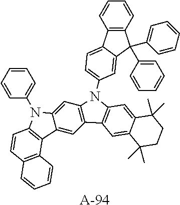 [SMILES: CC1(C)CCC(C)(C)c2cc3c(cc21)c1cc2c4c5ccccc5ccc4n(-c4ccccc4)c2cc1n3-c1ccc2c(c1)C(c1ccccc1)(c1ccccc1)c1ccccc1-2 ; "A-94"] | 809.4 | 57 |
| 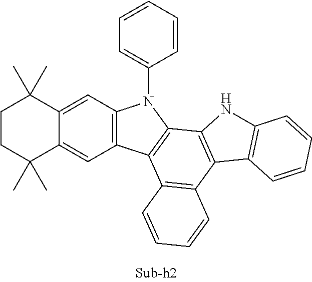 [SMILES: CC1(C)CCC(C)(C)c2cc3c(cc21)c1c2ccccc2c2c4ccccc4[nH]c2c1n3-c1ccccc1] | 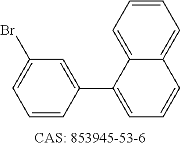 [SMILES: Brc1cccc(-c2cccc3ccccc23)c1] | 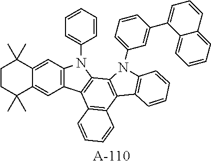 | 695.3 | 64 |
| 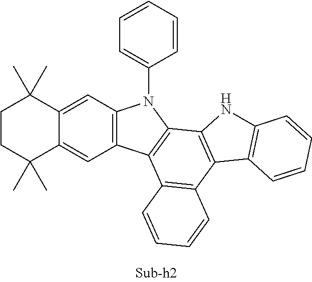 [SMILES: CC1(C)CCC(C)(C)c2cc3c(cc21)c1c2ccccc2c2c4ccccc4[nH]c2c1n3-c1ccccc1] | 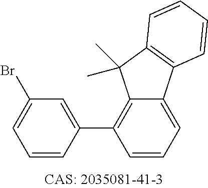 [SMILES: CC1(C)c2ccccc2-c2cccc(-c3cccc(Br)c3)c21] | 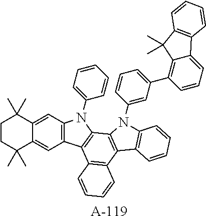 | 761.4 | 65 |
| 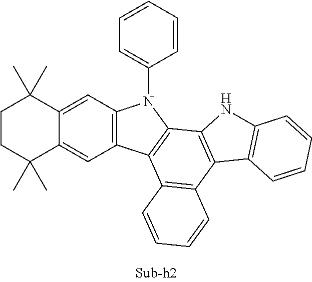 [SMILES: CC1(C)CCC(C)(C)c2cc3c(cc21)c1c2ccccc2c2c4ccccc4[nH]c2c1n3-c1ccccc1] | 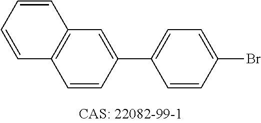 [SMILES: Brc1ccc(-c2ccc3ccccc3c2)cc1] | 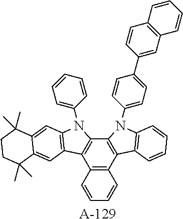 | 695.3 | 55 |
| 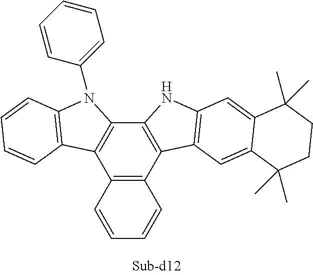 [SMILES: CC1(C)CCC(C)(C)c2cc3c(cc21)[nH]c1c3c2ccccc2c2c3ccccc3n(-c3ccccc3)c12] | 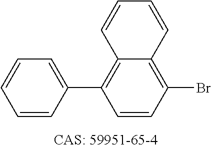 [SMILES: Brc1ccc(-c2ccccc2)c2ccccc12] | 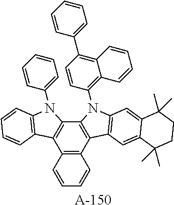 | 695.3 | 57 |
| 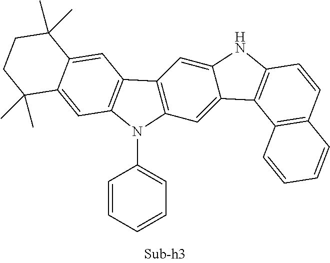 [SMILES: CC1(C)CCC(C)(C)c2cc3c(cc21)c1cc2[nH]c4ccc5ccccc5c4c2cc1n3-c1ccccc1] | 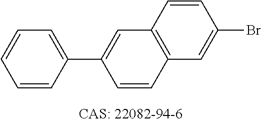 [SMILES: Brc1ccc2cc(-c3ccccc3)ccc2c1] | 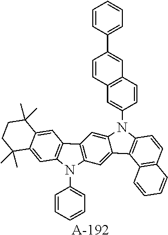 | 695.3 | 52 |
| 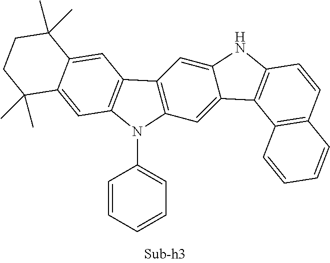 [SMILES: CC1(C)CCC(C)(C)c2cc3c(cc21)c1cc2[nH]c4ccc5ccccc5c4c2cc1n3-c1ccccc1] | 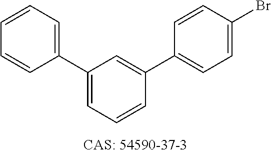 [SMILES: Brc1ccc(-c2cccc(-c3ccccc3)c2)cc1] | 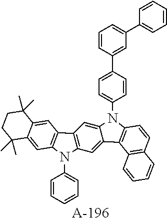 | 721.4 | 53 |
| 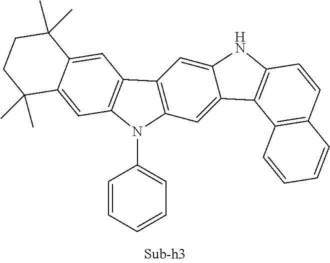 [SMILES: CC1(C)CCC(C)(C)c2cc3c(cc21)c1cc2[nH]c4ccc5ccccc5c4c2cc1n3-c1ccccc1] | 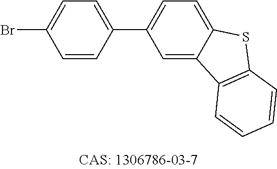 [SMILES: Brc1ccc(-c2ccc3sc4ccccc4c3c2)cc1] | 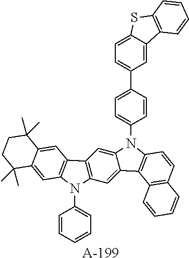 | 751.3 | 56 |
| 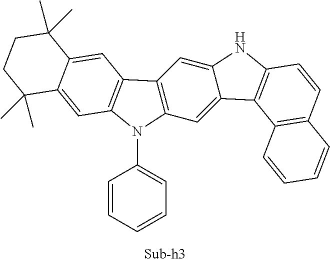 [SMILES: CC1(C)CCC(C)(C)c2cc3c(cc21)c1cc2[nH]c4ccc5ccccc5c4c2cc1n3-c1ccccc1] | 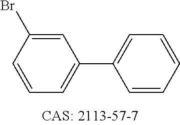 [SMILES: Brc1cccc(-c2ccccc2)c1] | 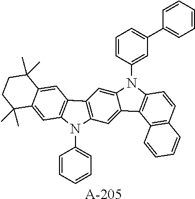 | 645.3 | 57 |
| 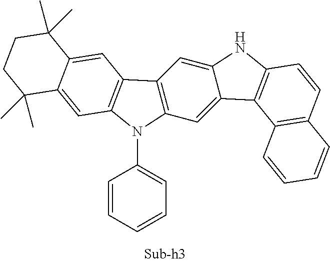 [SMILES: CC1(C)CCC(C)(C)c2cc3c(cc21)c1cc2[nH]c4ccc5ccccc5c4c2cc1n3-c1ccccc1] | 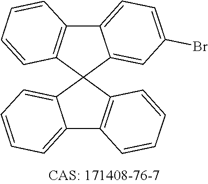 [SMILES: Brc1ccc2c(c1)C1(c3ccccc3-c3ccccc31)c1ccccc1-2] | 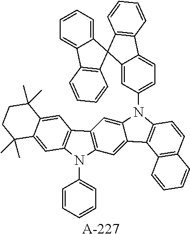 | 807.4 | 54 |
| 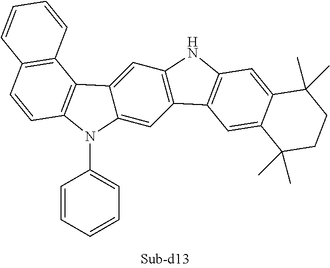 [SMILES: CC1(C)CCC(C)(C)c2cc3c(cc21)[nH]c1cc2c4c5ccccc5ccc4n(-c4ccccc4)c2cc13] | 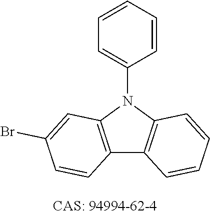 [SMILES: Brc1ccc2c3ccccc3n(-c3ccccc3)c2c1] | 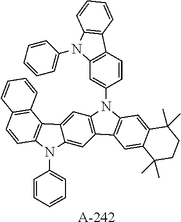 [SMILES: CC1(C)CCC(C)(C)c2cc3c(cc21)c1cc2c(cc1n3-c1ccc3c4ccccc4n(-c4ccccc4)c3c1)c1c3ccccc3ccc1n2-c1ccccc1 ; "A-242"] | 734.4 | 62 |
| 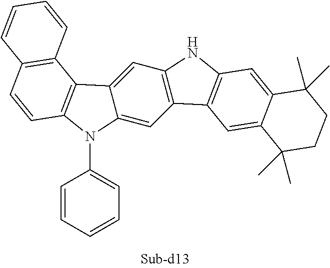 [SMILES: CC1(C)CCC(C)(C)c2cc3c(cc21)[nH]c1cc2c4c5ccccc5ccc4n(-c4ccccc4)c2cc13] | 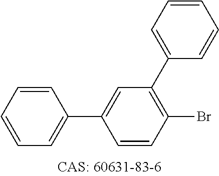 [SMILES: Brc1ccc(-c2ccccc2)cc1-c1ccccc1] | 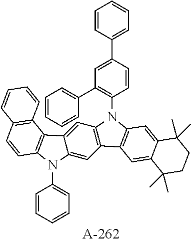 [SMILES: CC1(C)CCC(C)(C)c2cc3c(cc21)c1cc2c(cc1n3-c1ccc(-c3ccccc3)cc1-c1ccccc1)c1c3ccccc3ccc1n2-c1ccccc1 ; "A-262"] | 721.4 | 57 |
| 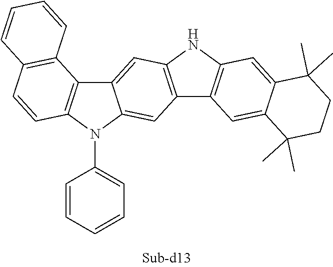 [SMILES: CC1(C)CCC(C)(C)c2cc3c(cc21)[nH]c1cc2c4c5ccccc5ccc4n(-c4ccccc4)c2cc13] | 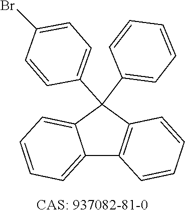 [SMILES: Brc1ccc(C2(c3ccccc3)c3ccccc3-c3ccccc32)cc1] | 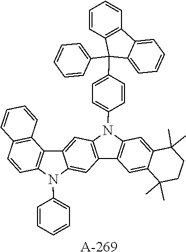 [SMILES: CC1(C)CCC(C)(C)c2cc3c(cc21)c1cc2c(cc1n3-c1ccc(C3(c4ccccc4)c4ccccc4-c4ccccc43)cc1)c1c3ccccc3ccc1n2-c1ccccc1 ; "A-269"] | 809.4 | 61 |
| 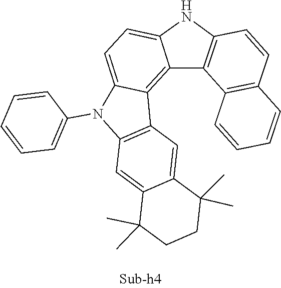 [SMILES: CC1(C)CCC(C)(C)c2cc3c(cc21)c1c2c(ccc1n3-c1ccccc1)[nH]c1ccc3ccccc3c12] | 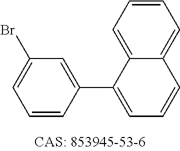 [SMILES: Brc1cccc(-c2cccc3ccccc23)c1] | 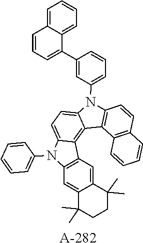 | 695.3 | 60 |
| 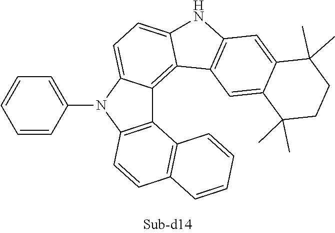 [SMILES: CC1(C)CCC(C)(C)c2cc3c(cc21)[nH]c1ccc2c(c13)c1c3ccccc3ccc1n2-c1ccccc1] | 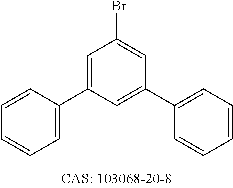 [SMILES: Brc1cc(-c2ccccc2)cc(-c2ccccc2)c1] | 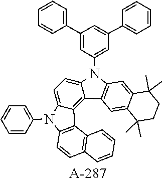 | 721.4 | 52 |
| 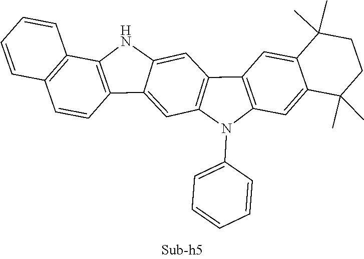 [SMILES: CC1(C)CCC(C)(C)c2cc3c(cc21)c1cc2[nH]c4c5ccccc5ccc4c2cc1n3-c1ccccc1] | 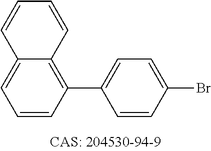 [SMILES: Brc1ccc(-c2cccc3ccccc23)cc1] | 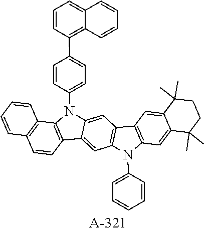 [SMILES: CC1(C)CCC(C)(C)c2cc3c(cc21)c1cc2c(cc1n3-c1ccccc1)c1ccc3ccccc3c1n2-c1ccc(-c2cccc3ccccc23)cc1 ; "A-321"] | 695.3 | 52 |
| 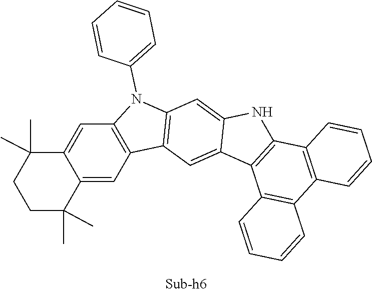 [SMILES: CC1(C)CCC(C)(C)c2cc3c(cc21)c1cc2c(cc1n3-c1ccccc1)[nH]c1c3ccccc3c3ccccc3c21] | 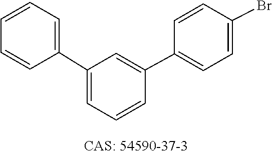 [SMILES: Brc1ccc(-c2cccc(-c3ccccc3)c2)cc1] | 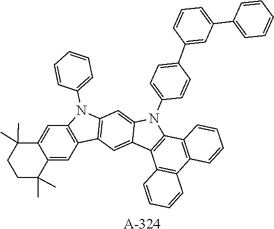 [SMILES: CC1(C)CCC(C)(C)c2cc3c(cc21)c1cc2c4c5ccccc5c5ccccc5c4n(-c4ccc(-c5cccc(-c6ccccc6)c5)cc4)c2cc1n3-c1ccccc1 ; "A-324"] | 771.4 | 57 |
| 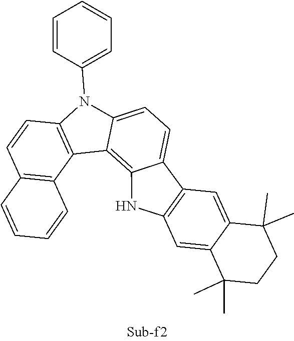 [SMILES: CC1(C)CCC(C)(C)c2cc3c(cc21)[nH]c1c3ccc2c1c1c3ccccc3ccc1n2-c1ccccc1] | 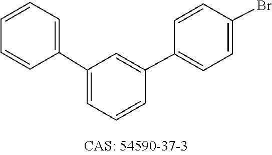 [SMILES: Brc1ccc(-c2cccc(-c3ccccc3)c2)cc1] | 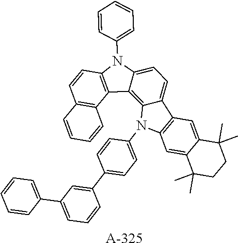 [SMILES: CC1(C)CCC(C)(C)c2cc3c(cc21)c1ccc2c(c4c5ccccc5ccc4n2-c2ccccc2)c1n3-c1ccc(-c2cccc(-c3ccccc3)c2)cc1 ; "A-325"] | 721.4 | 57 |
| 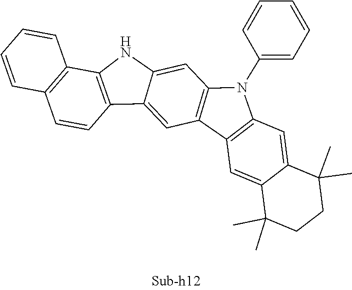 [SMILES: CC1(C)CCC(C)(C)c2cc3c(cc21)c1cc2c(cc1n3-c1ccccc1)[nH]c1c3ccccc3ccc21] | 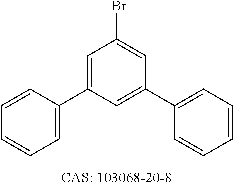 [SMILES: Brc1cc(-c2ccccc2)cc(-c2ccccc2)c1] | 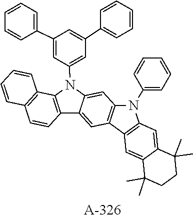 [SMILES: CC1(C)CCC(C)(C)c2cc3c(cc21)c1cc2c4ccc5ccccc5c4n(-c4cc(-c5ccccc5)cc(-c5ccccc5)c4)c2cc1n3-c1ccccc1 ; "A-326"] | 721.4 | 60 |
| 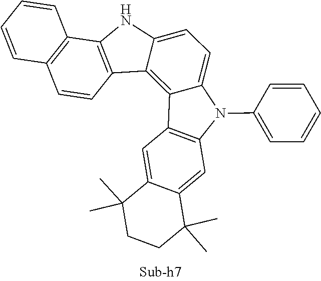 [SMILES: CC1(C)CCC(C)(C)c2cc3c(cc21)c1c2c(ccc1n3-c1ccccc1)[nH]c1c3ccccc3ccc12] | 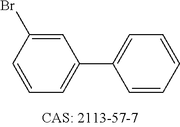 [SMILES: Brc1cccc(-c2ccccc2)c1] | 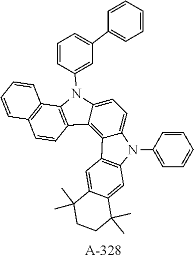 | 645.3 | 62 |
| 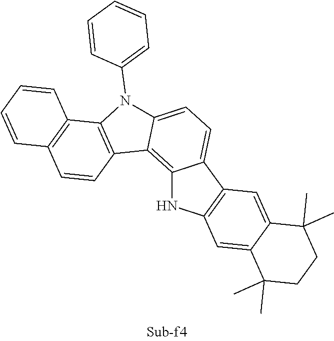 [SMILES: CC1(C)CCC(C)(C)c2cc3c(cc21)[nH]c1c3ccc2c1c1ccc3ccccc3c1n2-c1ccccc1] | 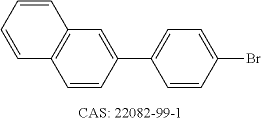 [SMILES: Brc1ccc(-c2ccc3ccccc3c2)cc1] | 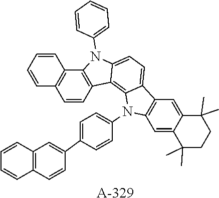 [SMILES: CC1(C)CCC(C)(C)c2cc3c(cc21)c1ccc2c(c4ccc5ccccc5c4n2-c2ccccc2)c1n3-c1ccc(-c2ccc3ccccc3c2)cc1 ; "A-329"] | 695.3 | 56 |
| 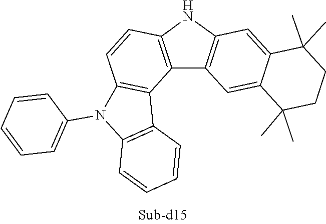 [SMILES: CC1(C)CCC(C)(C)c2cc3c(cc21)[nH]c1ccc2c(c4ccccc4n2-c2ccccc2)c13] | 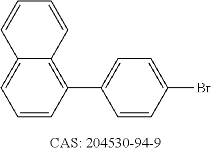 [SMILES: Brc1ccc(-c2cccc3ccccc23)cc1] | 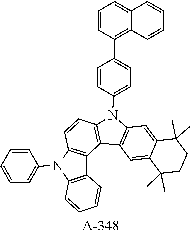 | 645.3 | 63 |
| 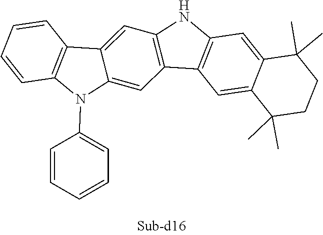 [SMILES: CC1(C)CCC(C)(C)c2cc3c(cc21)[nH]c1cc2c4ccccc4n(-c4ccccc4)c2cc13] | 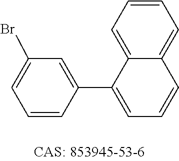 [SMILES: Brc1cccc(-c2cccc3ccccc23)c1] | 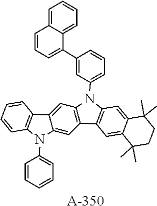 [SMILES: CC1(C)CCC(C)(C)c2cc3c(cc21)c1cc2c(cc1n3-c1cccc(-c3cccc4ccccc34)c1)c1ccccc1n2-c1ccccc1 ; "A-350"] | 645.3 | 63 |
| 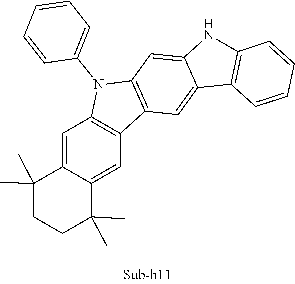 [SMILES: CC1(C)CCC(C)(C)c2cc3c(cc21)c1cc2c(cc1n3-c1ccccc1)[nH]c1ccccc12] | 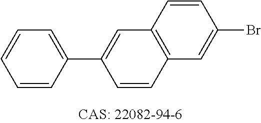 [SMILES: Brc1ccc2cc(-c3ccccc3)ccc2c1] | 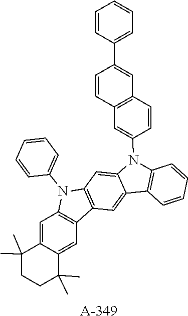 | 645.3 | 55 |
| 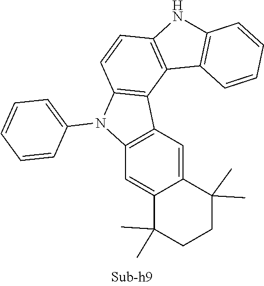 [SMILES: CC1(C)CCC(C)(C)c2cc3c(cc21)c1c2c(ccc1n3-c1ccccc1)[nH]c1ccccc12] | 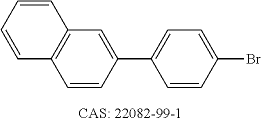 [SMILES: Brc1ccc(-c2ccc3ccccc3c2)cc1] | 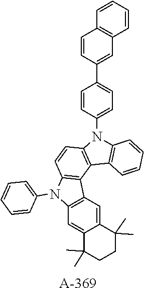 | 645.3 | 64 |
| 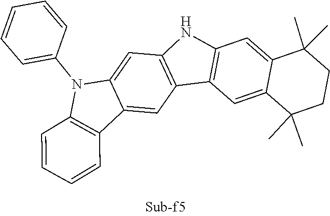 [SMILES: CC1(C)CCC(C)(C)c2cc3c(cc21)[nH]c1cc2c(cc13)c1ccccc1n2-c1ccccc1] | 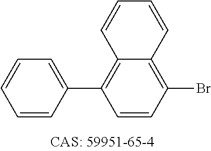 [SMILES: Brc1ccc(-c2ccccc2)c2ccccc12] | 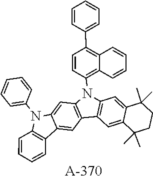 [SMILES: CC1(C)CCC(C)(C)c2cc3c(cc21)c1cc2c4ccccc4n(-c4ccccc4)c2cc1n3-c1ccc(-c2ccccc2)c2ccccc12 ; "A-370"] | 645.3 | 51 |
| 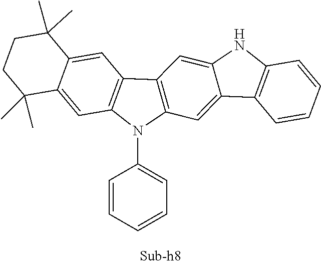 [SMILES: CC1(C)CCC(C)(C)c2cc3c(cc21)c1cc2[nH]c4ccccc4c2cc1n3-c1ccccc1] | 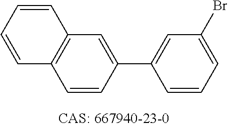 [SMILES: Brc1cccc(-c2ccc3ccccc3c2)c1] | 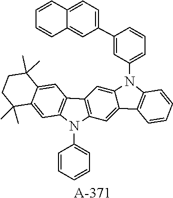 | 645.3 | 63 |
| 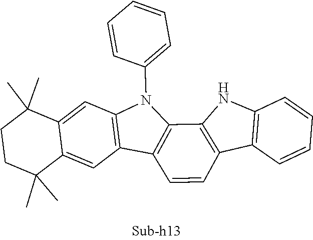 [SMILES: CC1(C)CCC(C)(C)c2cc3c(cc21)c1ccc2c4ccccc4[nH]c2c1n3-c1ccccc1] | 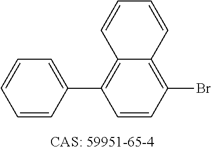 [SMILES: Brc1ccc(-c2ccccc2)c2ccccc12] | 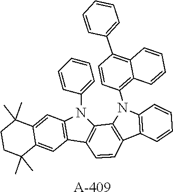 [SMILES: CC1(C)CCC(C)(C)c2cc3c(cc21)c1ccc2c4ccccc4n(-c4ccc(-c5ccccc5)c5ccccc45)c2c1n3-c1ccccc1 ; "A-409"] | 645.3 | 50 |
| 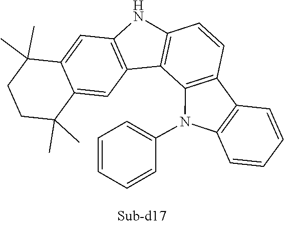 [SMILES: CC1(C)CCC(C)(C)c2cc3c(cc21)[nH]c1ccc2c4ccccc4n(-c4ccccc4)c2c13] | 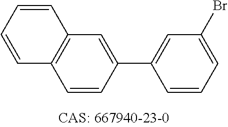 [SMILES: Brc1cccc(-c2ccc3ccccc3c2)c1] | 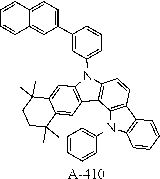 | 645.3 | 56 |

### Synthesis of Compound 1B-3

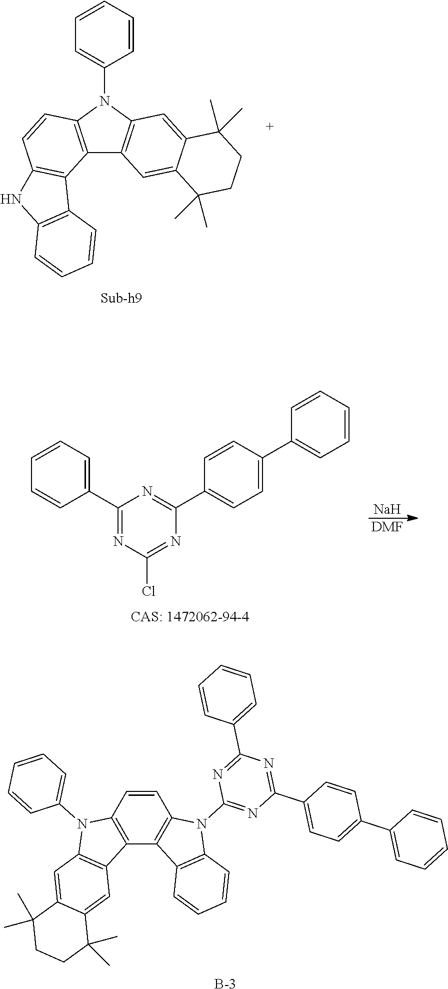

Under a nitrogen atmosphere, Sub-h9 (11.06 g, 25 mmol), 2-chloro-4-(biphenyl-4-yl)-6-phenyl-1,3,5-triazine (12.89 g, 37.5 mmol), and dried DMF (200 mL) were sequentially added to a 500 mL of three-necked flask. The system was cooled to −10° C., and sodium hydride (NaH, 60% w/w, 1.1 g, 55 mmol) was quickly added, followed by a reaction under stirring overnight. The reaction solution was poured into 200 mL of deionized water, stirred thoroughly for 30 minutes, and then filtered. The resulting solid was collected, washed with deionized water to neutral, and then rinsed with absolute ethanol (200 mL), obtaining a crude product. The crude product was purified by silica column chromatography with n-heptane/dichloromethane as a mobile phase to yielding Compound B-3 as a white solid (14.43 g, yield: 77%), m/z=750.4[M+H]⁺.

Compounds of the present application shown in Table 11 were synthesized respectively with reference to the synthesis of Compound B-3, with Sub-h9 being replaced with Reactant M shown in Table 11 and 2-chloro-4-(biphenyl-4-yl)-6-phenyl-1,3,5-triazine being replaced with Reactant N shown in Table 11.

TABLE 11
Synthesis of compounds of the present application

| | | | m/z | |
|---|---|---|---|---|
| | | Structure and No. of | ([M + | Yield |
| Reactant M | Reactant N | Compound | H]⁺) | (%) |
| 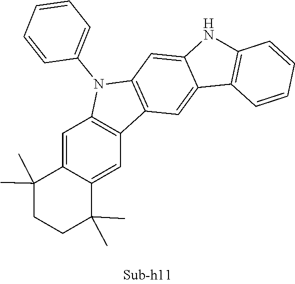 [SMILES: CC1(C)CCC(C)(C)c2cc3c(cc21)c1cc2c(cc1n3-c1ccccc1)[nH]c1ccccc12] | 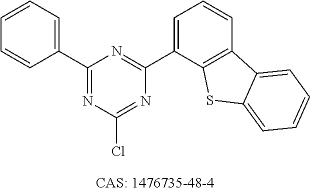 [SMILES: Clc1nc(-c2ccccc2)nc(-c2cccc3c2sc2ccccc23)n1] | 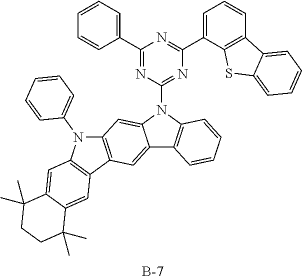 | 780.3 | 72 |
| 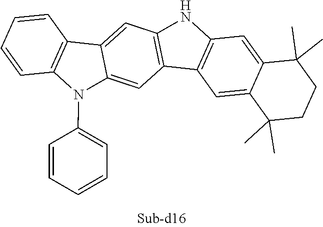 [SMILES: CC1(C)CCC(C)(C)c2cc3c(cc21)[nH]c1cc2c4ccccc4n(-c4ccccc4)c2cc13] | 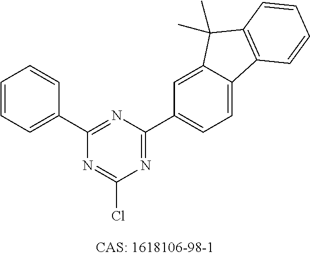 [SMILES: CC1(C)c2ccccc2-c2ccc(-c3nc(Cl)nc(-c4ccccc4)n3)cc21] | 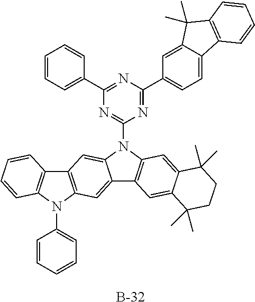 | 790.4 | 73 |
| 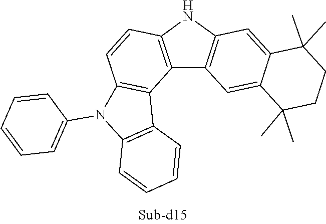 [SMILES: CC1(C)CCC(C)(C)c2cc3c(cc21)[nH]c1ccc2c(c4ccccc4n2-c2ccccc2)c13] | 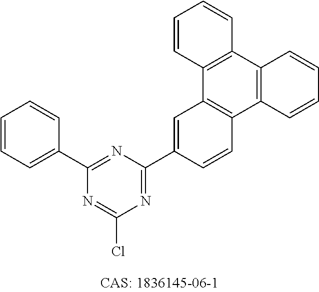 [SMILES: Clc1nc(-c2ccccc2)nc(-c2ccc3c4ccccc4c4ccccc4c3c2)n1] | 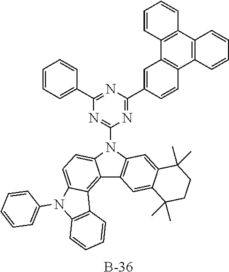 | 824.4 | 73 |
| 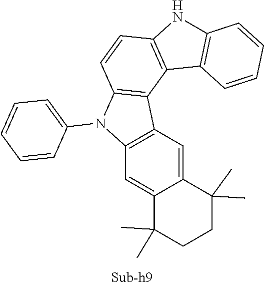 [SMILES: CC1(C)CCC(C)(C)c2cc3c(cc21)c1c2c(ccc1n3-c1ccccc1)[nH]c1ccccc12] | 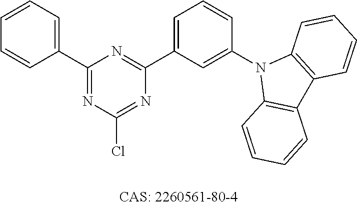 [SMILES: Clc1nc(-c2ccccc2)nc(-c2cccc(-n3c4ccccc4c4ccccc43)c2)n1] | 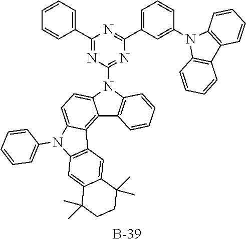 | 839.4 | 60 |
| 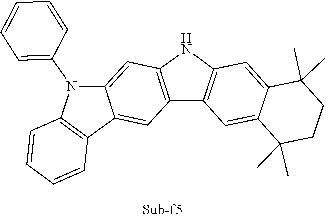 [SMILES: CC1(C)CCC(C)(C)c2cc3c(cc21)[nH]c1cc2c(cc13)c1ccccc1n2-c1ccccc1] | 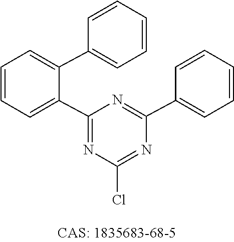 [SMILES: Clc1nc(-c2ccccc2)nc(-c2ccccc2-c2ccccc2)n1] | 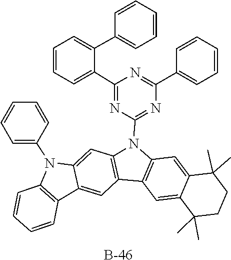 | 750.4 | 60 |
| 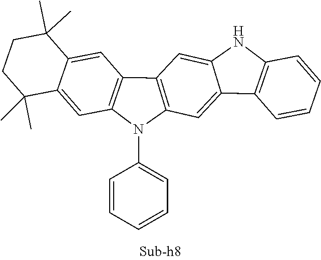 [SMILES: CC1(C)CCC(C)(C)c2cc3c(cc21)c1cc2[nH]c4ccccc4c2cc1n3-c1ccccc1] | 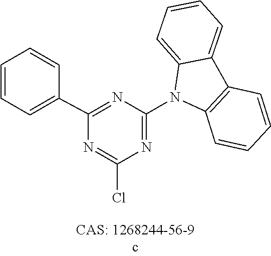 [SMILES: Clc1nc(-c2ccccc2)nc(-n2c3ccccc3c3ccccc32)n1] | 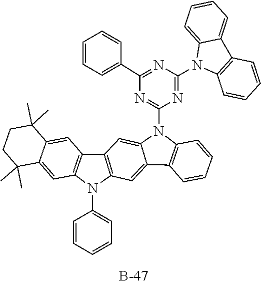 | 763.4 | 68 |
| 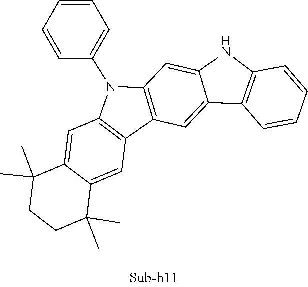 [SMILES: CC1(C)CCC(C)(C)c2cc3c(cc21)c1cc2c(cc1n3-c1ccccc1)[nH]c1ccccc12] | 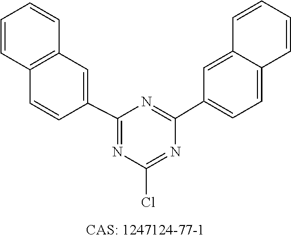 [SMILES: Clc1nc(-c2ccc3ccccc3c2)nc(-c2ccc3ccccc3c2)n1] | 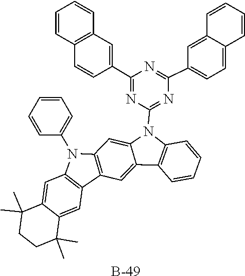 | 774.4 | 69 |
| 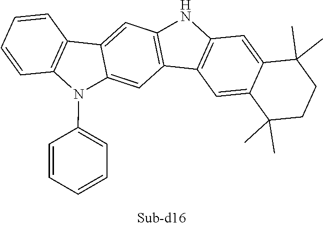 [SMILES: CC1(C)CCC(C)(C)c2cc3c(cc21)[nH]c1cc2c4ccccc4n(-c4ccccc4)c2cc13] | 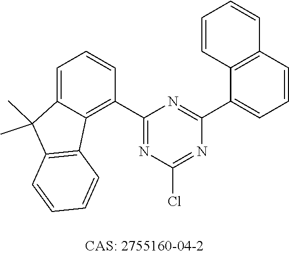 [SMILES: CC1(C)c2ccccc2-c2c(-c3nc(Cl)nc(-c4cccc5ccccc45)n3)cccc21] | 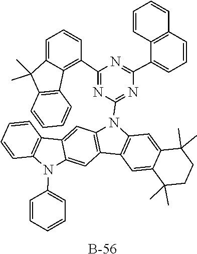 | 840.4 | 63 |
| 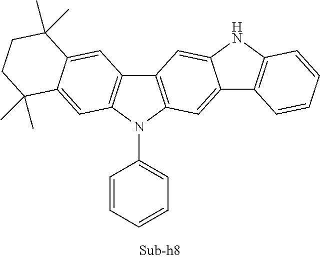 [SMILES: CC1(C)CCC(C)(C)c2cc3c(cc21)c1cc2[nH]c4ccccc4c2cc1n3-c1ccccc1] | 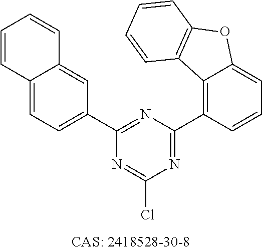 [SMILES: Clc1nc(-c2ccc3ccccc3c2)nc(-c2cccc3oc4ccccc4c23)n1] | 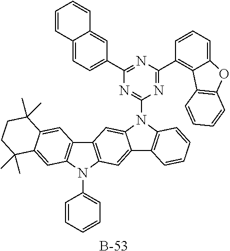 | 814.4 | 69 |
| 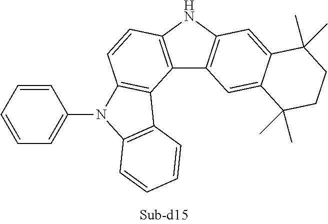 [SMILES: CC1(C)CCC(C)(C)c2cc3c(cc21)[nH]c1ccc2c(c4ccccc4n2-c2ccccc2)c13] | 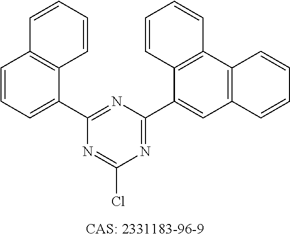 [SMILES: Clc1nc(-c2cccc3ccccc23)nc(-c2cc3ccccc3c3ccccc23)n1] | 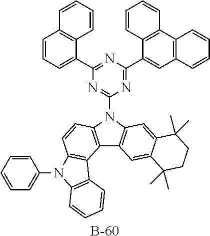 | 824.4 | 68 |
| 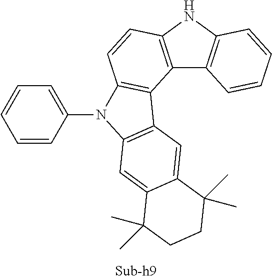 [SMILES: CC1(C)CCC(C)(C)c2cc3c(cc21)c1c2c(ccc1n3-c1ccccc1)[nH]c1ccccc12] | 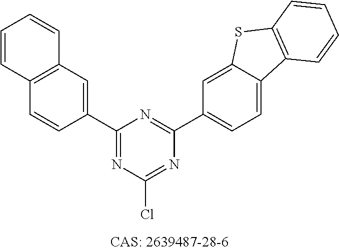 [SMILES: Clc1nc(-c2ccc3ccccc3c2)nc(-c2ccc3c(c2)sc2ccccc23)n1] | 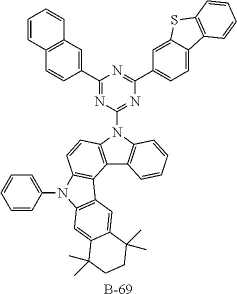 | 830.3 | 70 |
| 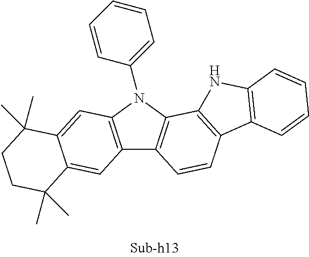 [SMILES: CC1(C)CCC(C)(C)c2cc3c(cc21)c1ccc2c4ccccc4[nH]c2c1n3-c1ccccc1] | 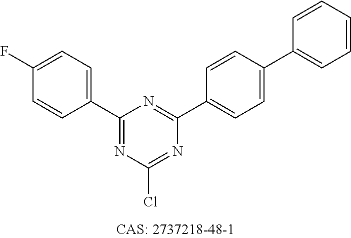 [SMILES: Fc1ccc(-c2nc(Cl)nc(-c3ccc(-c4ccccc4)cc3)n2)cc1] | 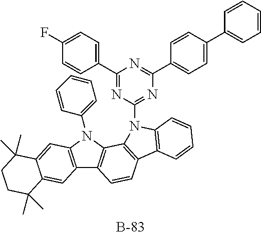 | 768.4 | 61 |
| 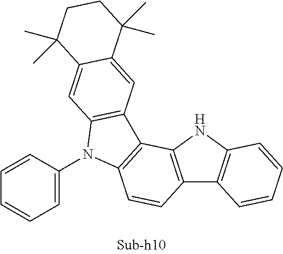 [SMILES: CC1(C)CCC(C)(C)c2cc3c(cc21)c1c2[nH]c4ccccc4c2ccc1n3-c1ccccc1] | 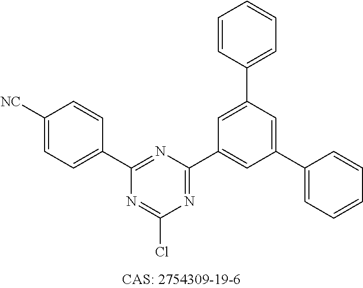 [SMILES: N#Cc1ccc(-c2nc(Cl)nc(-c3cc(-c4ccccc4)cc(-c4ccccc4)c3)n2)cc1] | 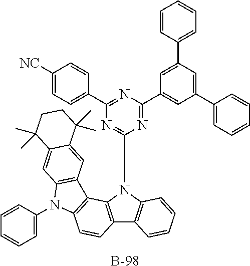 | 851.4 | 60 |
| 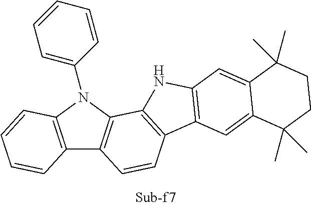 [SMILES: CC1(C)CCC(C)(C)c2cc3c(cc21)[nH]c1c3ccc2c3ccccc3n(-c3ccccc3)c21] | 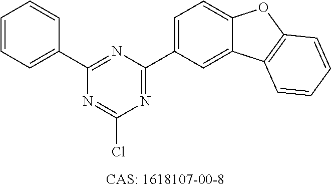 [SMILES: Clc1nc(-c2ccccc2)nc(-c2ccc3oc4ccccc4c3c2)n1] | 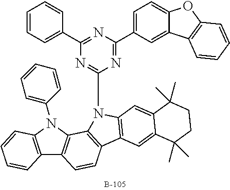 [SMILES: CC1(C)CCC(C)(C)c2cc3c(cc21)c1ccc2c4ccccc4n(-c4ccccc4)c2c1n3-c1nc(-c2ccccc2)nc(-c2ccc3oc4ccccc4c3c2)n1] | 764.3 | 71 |
| 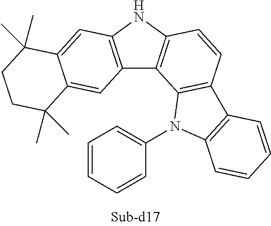 [SMILES: CC1(C)CCC(C)(C)c2cc3c(cc21)[nH]c1ccc2c4ccccc4n(-c4ccccc4)c2c13] | 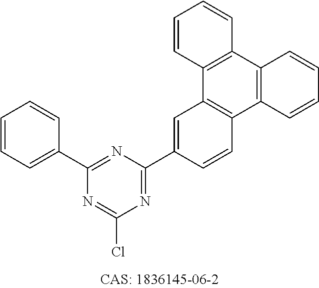 [SMILES: Clc1nc(-c2ccccc2)nc(-c2ccc3c4ccccc4c4ccccc4c3c2)n1] | 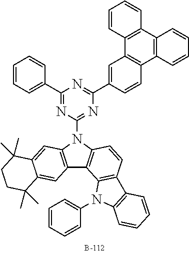 [SMILES: CC1(C)CCC(C)(C)c2cc3c(cc21)c1c(ccc2c4ccccc4n(-c4ccccc4)c21)n3-c1nc(-c2ccccc2)nc(-c2ccc3c4ccccc4c4ccccc4c3c2)n1] | 824.4 | 69 |
| 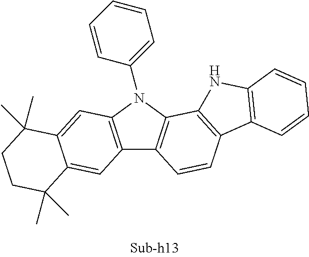 [SMILES: CC1(C)CCC(C)(C)c2cc3c(cc21)c1ccc2c4ccccc4[nH]c2c1n3-c1ccccc1] | 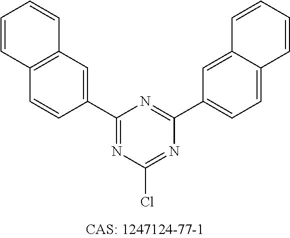 [SMILES: Clc1nc(-c2ccc3ccccc3c2)nc(-c2ccc3ccccc3c2)n1] | 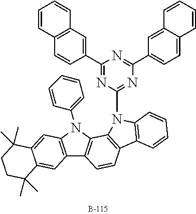 [SMILES: CC1(C)CCC(C)(C)c2cc3c(cc21)c1ccc2c4ccccc4n(-c4nc(-c5ccc6ccccc6c5)nc(-c5ccc6ccccc6c5)n4)c2c1n3-c1ccccc1] | 774.4 | 67 |
| 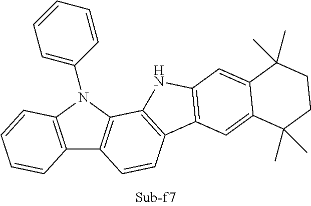 [SMILES: CC1(C)CCC(C)(C)c2cc3c(cc21)[nH]c1c3ccc2c3ccccc3n(-c3ccccc3)c21] | 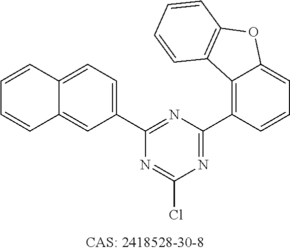 [SMILES: Clc1nc(-c2ccc3ccccc3c2)nc(-c2cccc3oc4ccccc4c23)n1] | 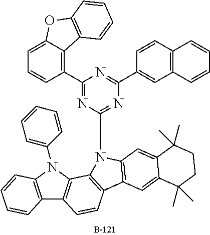 [SMILES: CC1(C)CCC(C)(C)c2cc3c(cc21)c1ccc2c4ccccc4n(-c4ccccc4)c2c1n3-c1nc(-c2ccc3ccccc3c2)nc(-c2cccc3oc4ccccc4c23)n1] | 814.4 | 75 |
| 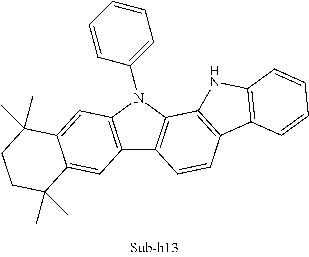 [SMILES: CC1(C)CCC(C)(C)c2cc3c(cc21)c1ccc2c4ccccc4[nH]c2c1n3-c1ccccc1] | 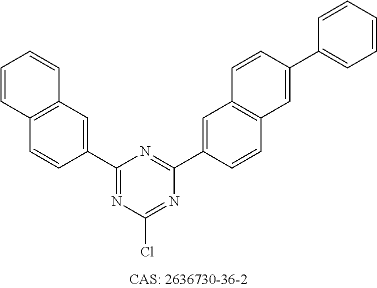 [SMILES: Clc1nc(-c2ccc3ccccc3c2)nc(-c2ccc3cc(-c4ccccc4)ccc3c2)n1] | 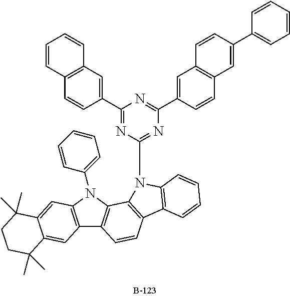 [SMILES: CC1(C)CCC(C)(C)c2cc3c(cc21)c1ccc2c4ccccc4n(-c4nc(-c5ccc6ccccc6c5)nc(-c5ccc6cc(-c7ccccc7)ccc6c5)n4)c2c1n3-c1ccccc1] | 850.4 | 65 |
| 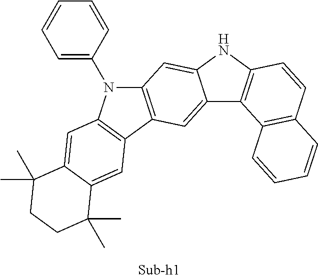 [SMILES: CC1(C)CCC(C)(C)c2cc3c(cc21)c1cc2c(cc1n3-c1ccccc1)[nH]c1ccc3ccccc3c12] | 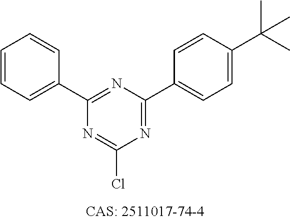 [SMILES: CC(C)(C)c1ccc(-c2nc(Cl)nc(-c3ccccc3)n2)cc1] | 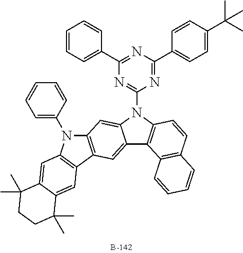 [SMILES: CC(C)(C)c1ccc(-c2nc(-c3ccccc3)nc(-n3c4cc5c(cc4c4c6ccccc6ccc43)c3cc4c(cc3n5-c3ccccc3)C(C)(C)CCC4(C)C)n2)cc1] | 780.4 | 71 |
| 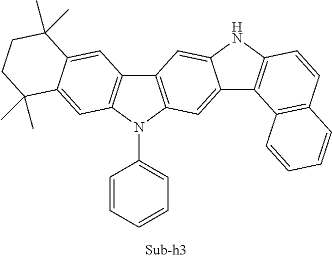 [SMILES: CC1(C)CCC(C)(C)c2cc3c(cc21)c1cc2[nH]c4ccc5ccccc5c4c2cc1n3-c1ccccc1] | 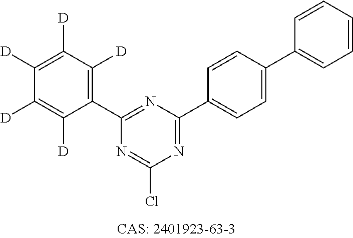 [SMILES: [2H]c1c([2H])c([2H])c(-c2nc(Cl)nc(-c3ccc(-c4ccccc4)cc3)n2)c([2H])c1[2H]] | 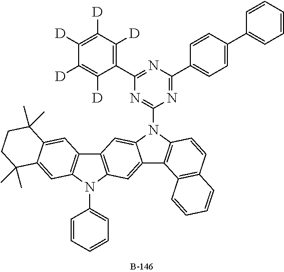 [SMILES: [2H]c1c([2H])c([2H])c(-c2nc(-c3ccc(-c4ccccc4)cc3)nc(-n3c4cc5c6cc7c(cc6n(-c6ccccc6)c5cc4c4c5ccccc5ccc43)C(C)(C)CCC7(C)C)n2)c([2H])c1[2H]] | 805.4 | 73 |
| 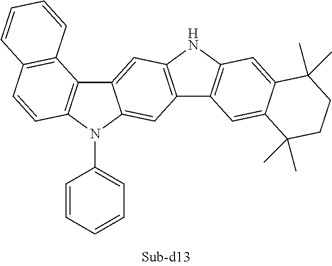 [SMILES: CC1(C)CCC(C)(C)c2cc3c(cc21)[nH]c1cc2c4c5ccccc5ccc4n(-c4ccccc4)c2cc13] | 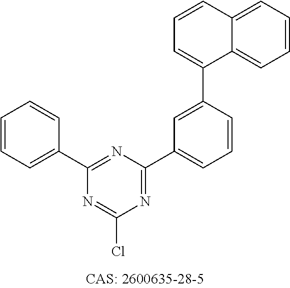 [SMILES: Clc1nc(-c2ccccc2)nc(-c2cccc(-c3cccc4ccccc34)c2)n1] | 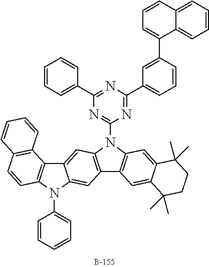 [SMILES: CC1(C)CCC(C)(C)c2cc3c(cc21)c1cc2c(cc1n3-c1nc(-c3ccccc3)nc(-c3cccc(-c4cccc5ccccc45)c3)n1)c1c3ccccc3ccc1n2-c1ccccc1] | 850.4 | 67 |
| 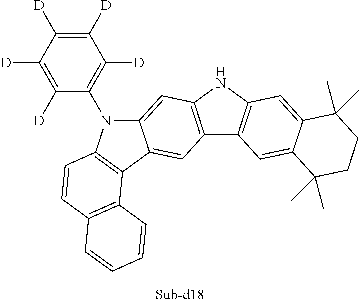 [SMILES: [2H]c1c([2H])c([2H])c(-n2c3cc4[nH]c5cc6c(cc5c4cc3c3c4ccccc4ccc32)C(C)(C)CCC6(C)C)c([2H])c1[2H]] | 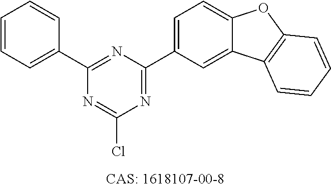 [SMILES: Clc1nc(-c2ccccc2)nc(-c2ccc3oc4ccccc4c3c2)n1] | 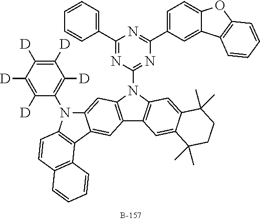 [SMILES: [2H]c1c([2H])c([2H])c(-n2c3cc4c(cc3c3c5ccccc5ccc32)c2cc3c(cc2n4-c2nc(-c4ccccc4)nc(-c4ccc5oc6ccccc6c5c4)n2)C(C)(C)CCC3(C)C)c([2H])c1[2H]] | 819.4 | 69 |
| 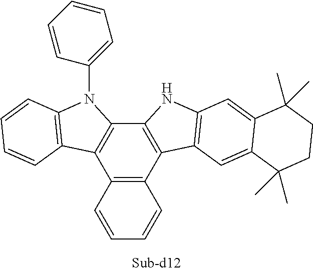 [SMILES: CC1(C)CCC(C)(C)c2cc3c(cc21)[nH]c1c3c2ccccc2c2c3ccccc3n(-c3ccccc3)c12] | 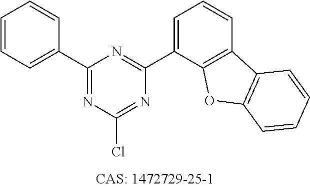 [SMILES: Clc1nc(-c2ccccc2)nc(-c2cccc3c2oc2ccccc23)n1] | 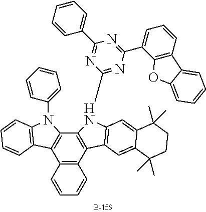 | 815.4 | 60 |
| 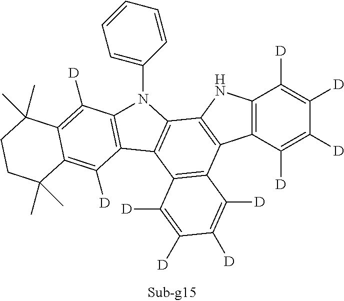 [SMILES: [2H]c1c([2H])c([2H])c2c([nH]c3c2c2c([2H])c([2H])c([2H])c([2H])c2c2c4c([2H])c5c(c([2H])c4n(-c4ccccc4)c32)C(C)(C)CCC5(C)C)c1[2H]] | 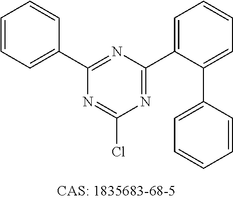 [SMILES: Clc1nc(-c2ccccc2)nc(-c2ccccc2-c2ccccc2)n1] | 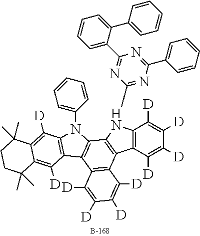 | 810.4 | 62 |
| 1 | | | | |
| 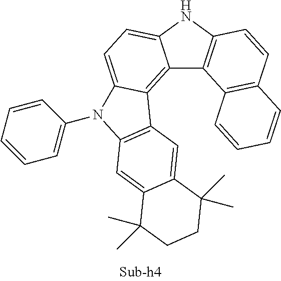 [SMILES: CC1(C)CCC(C)(C)c2cc3c(cc21)c1c2c(ccc1n3-c1ccccc1)[nH]c1ccc3ccccc3c12] | 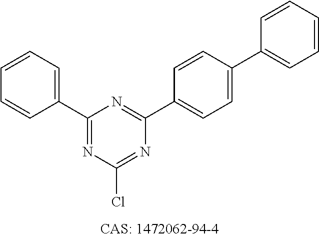 [SMILES: Clc1nc(-c2ccccc2)nc(-c2ccc(-c3ccccc3)cc2)n1] | 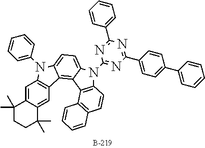 [SMILES: CC1(C)CCC(C)(C)c2cc3c(cc21)c1c2c4c5ccccc5ccc4n(-c4nc(-c5ccccc5)nc(-c5ccc(-c6ccccc6)cc5)n4)c2ccc1n3-c1ccccc1] | 800.4 | 70 |
| 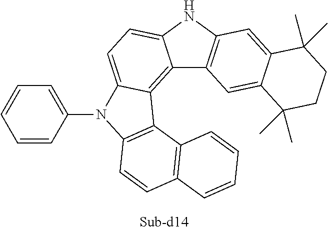 [SMILES: CC1(C)CCC(C)(C)c2cc3c(cc21)[nH]c1ccc2c(c13)c1c3ccccc3ccc1n2-c1ccccc1] | 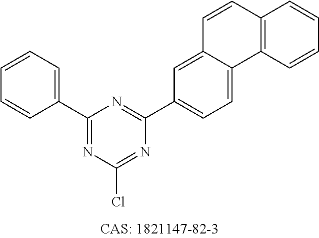 [SMILES: Clc1nc(-c2ccccc2)nc(-c2ccc3c(ccc4ccccc43)c2)n1] | 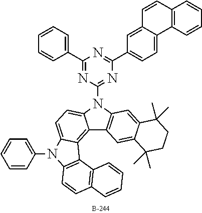 [SMILES: CC1(C)CCC(C)(C)c2cc3c(cc21)c1c2c4c5ccccc5ccc4n(-c4ccccc4)c2ccc1n3-c1nc(-c2ccccc2)nc(-c2ccc3c(ccc4ccccc43)c2)n1] | 824.4 | 60 |

### Synthesis of Compound B-172

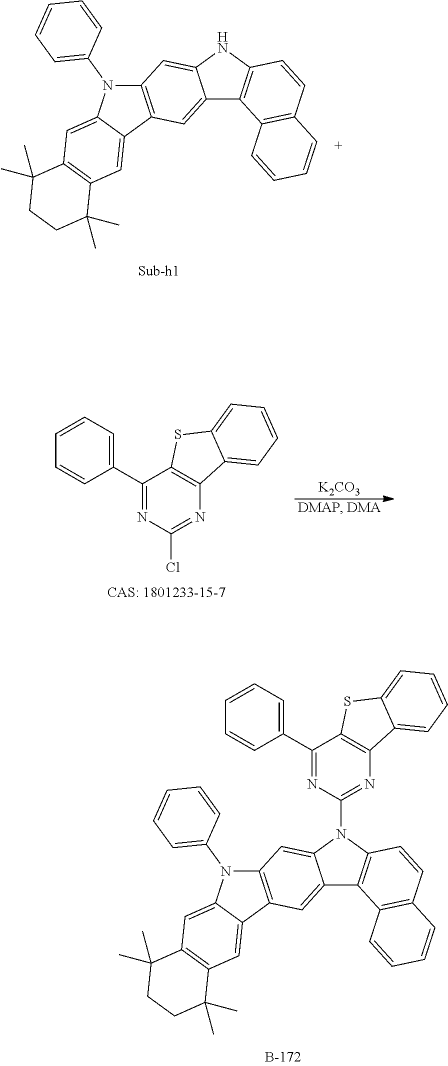

Under a nitrogen atmosphere, Sub-h1 (14.78 g, 30 mmol), CAS: 1801233-15-7 (9.8 g, 33 mmol), anhydrous potassium carbonate (K₂CO₃, 4.15 g, 30 mmol), 4-dimethylaminopyridine (1.83 g, 15 mmol), and N,N-dimethylacetamide (100 mL) were added sequentially to a 500 mL of three-necked flask, and heated to a refluxing temperature for a reaction for 16 hours. After being cooled to room temperature, the system was extracted with dichloromethane (100 mL×3 times). The resulting organic phases were combined and dried over anhydrous sodium sulfate, followed by filtration and vacuum distillation to remove the solvent, obtaining a crude product. The crude product was purified by silica column chromatography with n-heptane/dichloromethane as a mobile phase to yield Compound B-172 as a white solid (13.10 g, yield: 58%), m/z=753.3[M+H]⁺.

Compounds of the present application shown in Table 12 were synthesized respectively with reference to the synthesis of Compound B-172, with Sub-h1 being replaced with Reactant O shown in Table 12 and with CAS:1801233-15-7 being replaced with reactant P shown in Table 12.

TABLE 12
Synthesis of compounds of the present application

| | | | m/z | |
|---|---|---|---|---|
| | | Structure and No. of | ([M + | Yield |
| Reactant O | Reactant P | Compound | H]⁺) | (%) |
| 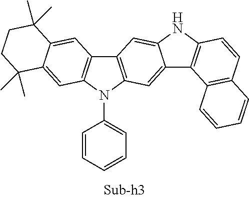 [SMILES: CC1(C)CCC(C)(C)c2cc3c(cc21)c1cc2[nH]c4ccc5ccccc5c4c2cc1n3-c1ccccc1] | 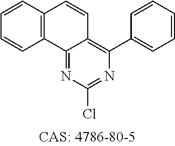 [SMILES: Clc1nc(-c2ccccc2)c2ccc3ccccc3c2n1] | 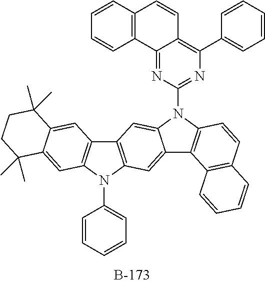 [SMILES: CC1(C)CCC(C)(C)c2cc3c(cc21)c1cc2c(cc1n3-c1ccccc1)c1c3ccccc3ccc1n2-c1nc(-c2ccccc2)c2ccc3ccccc3c2n1] | 747.3 | 49 |
| 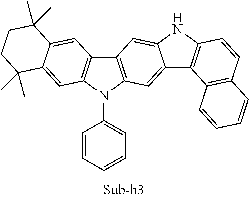 [SMILES: CC1(C)CCC(C)(C)c2cc3c(cc21)c1cc2[nH]c4ccc5ccccc5c4c2cc1n3-c1ccccc1] | 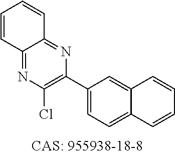 [SMILES: Clc1nc2ccccc2nc1-c1ccc2ccccc2c1] | 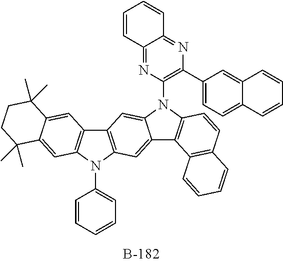 [SMILES: CC1(C)CCC(C)(C)c2cc3c(cc21)c1cc2c(cc1n3-c1ccccc1)c1c3ccccc3ccc1n2-c1nc2ccccc2nc1-c1ccc2ccccc2c1] | 747.3 | 45 |
| 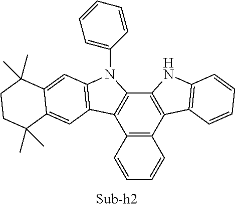 [SMILES: CC1(C)CCC(C)(C)c2cc3c(cc21)c1c2ccccc2c2c4ccccc4[nH]c2c1n3-c1ccccc1] | 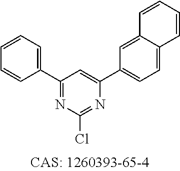 [SMILES: Clc1nc(-c2ccccc2)cc(-c2ccc3ccccc3c2)n1] | 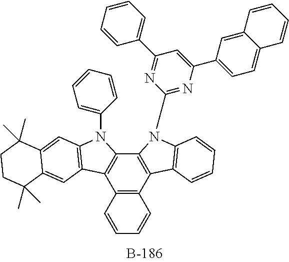 [SMILES: CC1(C)CCC(C)(C)c2cc3c(cc21)c1c2ccccc2c2c4ccccc4n(-c4nc(-c5ccccc5)cc(-c5ccc6ccccc6c5)n4)c2c1n3-c1ccccc1] | 773.4 | 53 |
| 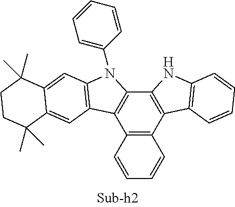 [SMILES: CC1(C)CCC(C)(C)c2cc3c(cc21)c1c2ccccc2c2c4ccccc4[nH]c2c1n3-c1ccccc1] | 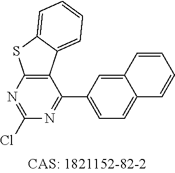 [SMILES: Clc1nc(-c2ccc3ccccc3c2)c2c(n1)sc1ccccc12] | 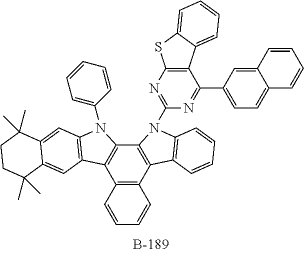 [SMILES: CC1(C)CCC(C)(C)c2cc3c(cc21)c1c2ccccc2c2c4ccccc4n(-c4nc(-c5ccc6ccccc6c5)c5c(n4)sc4ccccc45)c2c1n3-c1ccccc1] | 803.3 | 46 |
| 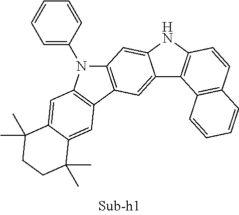 [SMILES: CC1(C)CCC(C)(C)c2cc3c(cc21)c1cc2c(cc1n3-c1ccccc1)[nH]c1ccc3ccccc3c12] | 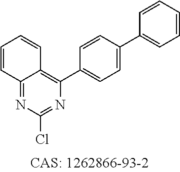 [SMILES: Clc1nc(-c2ccc(-c3ccccc3)cc2)c2ccccc2n1] | 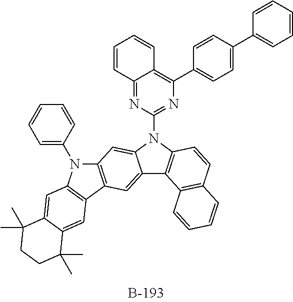 [SMILES: CC1(C)CCC(C)(C)c2cc3c(cc21)c1cc2c4c5ccccc5ccc4n(-c4nc(-c5ccc(-c6ccccc6)cc5)c5ccccc5n4)c2cc1n3-c1ccccc1] | 773.4 | 45 |
| 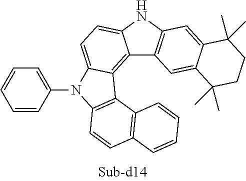 [SMILES: CC1(C)CCC(C)(C)c2cc3c(cc21)[nH]c1ccc2c(c13)c1c3ccccc3ccc1n2-c1ccccc1] | 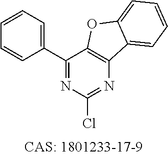 [SMILES: Clc1nc(-c2ccccc2)c2oc3ccccc3c2n1] | 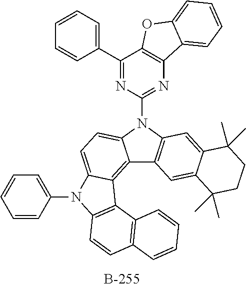 [SMILES: CC1(C)CCC(C)(C)c2cc3c(cc21)c1c2c4c5ccccc5ccc4n(-c4ccccc4)c2ccc1n3-c1nc(-c2ccccc2)c2oc3ccccc3c2n1] | 737.3 | 49 |
| 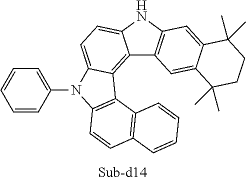 [SMILES: CC1(C)CCC(C)(C)c2cc3c(cc21)[nH]c1ccc2c(c13)c1c3ccccc3ccc1n2-c1ccccc1] | 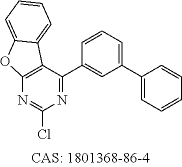 [SMILES: Clc1nc(-c2cccc(-c3ccccc3)c2)c2c(n1)oc1ccccc12] | 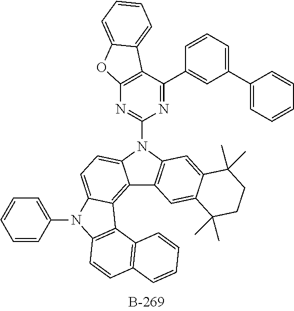 [SMILES: CC1(C)CCC(C)(C)c2cc3c(cc21)c1c2c4c5ccccc5ccc4n(-c4ccccc4)c2ccc1n3-c1nc(-c2cccc(-c3ccccc3)c2)c2c(n1)oc1ccccc12] | 813.4 | 54 |

NMR data of Compound A-8: ¹H-NMR (400 MHz, CD₂Cl₂) δ ppm 8.91 (d, 1H), 8.21 (d, 1H), 7.99 (d, 1H), 7.91 (d, 1H), 7.87 (d, 1H), 7.77 (d, 1H), 7.74 (d, 1H), 7.63 (t, 1H), 7.59-7.35 (m, 15H), 7.30 (t, 1H), 7.27 (s, 1H), 6.88 (s, 1H), 1.77 (s, 4H), 1.39 (d, 12H).

NMR data of Compound A-129: ¹H-NMR (400 MHz, CD₂Cl₂) δ ppm 8.22 (d, 1H), 7.96 (s, 1H), 7.89-7.77 (m, 4H), 7.70-7.63 (m, 6H), 7.58 (t, 2H), 7.52-7.40 (m, 5H), 7.38-7.25 (m, 5H), 7.15 (d, 2H), 1.74 (s, 4H), 1.34 (d, 12H).

NMR data of Compound A-324: ¹H-NMR (400 MHz, CD₂Cl₂) δ ppm 8.92 (s, 1H), 8.60 (d, 1H), 8.22 (d, 1H), 8.12 (d, 1H), 8.06 (s, 1H), 7.68 (d, 2H), 7.63 (d, 1H), 7.59-7.26 (m, 18H), 7.19 (t, 1H), 7/07 (d, 2H), 6.97 (s, 1H), 1.78 (s, 4H), 1.40 (d, 12H).

NMR data of Compound B-157: ¹H-NMR (400 MHz, CD₂Cl₂) δ ppm 10.02 (s, 1H), 8.84 (s, 1H), 8.61 (d, 1H), 8.52 (d, 2H), 8.21 (d, 1H), 8.10 (s, 1H), 8.04 (d, 1H), 8.00 (d, 1H), 7.91 (d, 1H), 7.84 (s, 1H), 7.81 (d, 1H), 7.68 (s, 1H), 7.66-7.31 (m, 9H), 1.75 (s, 4H), 1.36 (d, 12H).

NMR data of Compound B-182: ¹H-NMR (400 MHz, CD₂Cl₂) δ ppm 8.54 (d, 1H), 8.36 (s, 1H), 8.21 (s, 1H), 8.15 (s, 1H), 8.12-7.98 (m, 6H), 7.95 (d, 1H), 7.91-7.80 (m, 4H), 7.66-7.46 (m, 8H), 7.44-7.38 (m, 2H), 7.27 (s, 1H), 1.77 (s, 4H), 1.38 (d, 12H).

### Fabrication and Evaluation of Organic Electroluminescent Devices:

### Example 1: Red Light Organic Electroluminescent Device

First, anode pretreatment was performed in the following process. A surface of an ITO/Ag/ITO substrate, with thicknesses of ITO/Ag/ITO being 100 Å, 1000 Å, and 100 Å, respectively, was treated using ultraviolet ozone and O₂:N₂ plasma to increase the work function of the anode, and then cleaned with an organic solvent to remove impurities and oil on the ITO substrate.

Compound PD was deposited by vacuum evaporation on the experimental substrate (anode) to form a hole injection layer (HIL) having a thickness of 100 Å, and then Compound HT-1 was deposited by vacuum evaporation on the hole injection layer to form a first hole transport layer having a thickness of 1080 Å.

Compound HT-2 was deposited by vacuum evaporation on the first hole transport layer to form a second hole transport layer having a thickness of 890 Å.

Next, Compound A-3, Compound RH—N, and Compound RD-1 were co-deposited by evaporation on the second hole transport layer at an evaporation rate ratio of 49%:49%:2% to form a red light-emitting light emitting layer (EML) having a thickness of 400 Å.

Compound ET-1 and Compound LiQ were mixed at a 1:1 weight ratio and deposited by evaporation on the light emitting layer to form an electron transport layer (ETL) having a thickness of 350 Å; Yb was deposited by evaporation on the electron transport layer to form an electron injection layer (EIL) having a thickness of 10 Å; and then magnesium (Mg) and silver (Ag) were mixed at an evaporation rate ratio of 1:9, and deposited by vacuum evaporation on the electron injection layer to form a cathode having a thickness of 130 Å.

Further, Compound CPL-1 was deposited by vacuum evaporation on the above cathode to form a capping layer having a thickness of 805 Å, completing the fabrication of a red-light emitting organic electroluminescent device.

### Examples 2-45

Organic electroluminescent devices were fabricated by the same method as used in Example 1, except that Compound A-3 in Example 1 was replaced with Compound X shown in the following Table 13 when an light emitting layer was formed.

### Comparative Examples 1-3

Organic electroluminescent devices were fabricated by the same method as used in Example 1, except that Compound A-3 in Example 1 was replaced with a respective one of Compound A, Compound B, and Compound C when an light emitting layer was formed.

In various examples and comparative examples, structures of the compounds used are as follows:

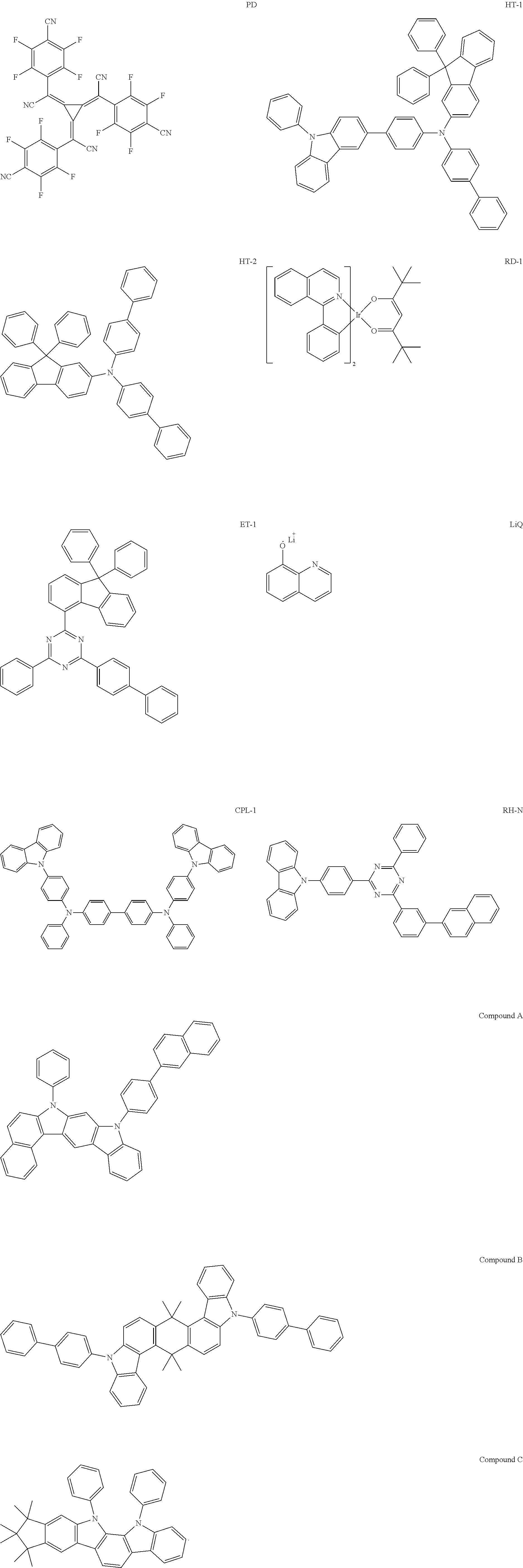

The red-light organic electroluminescent devices fabricated in Examples 1-45 and Comparative Examples 1-3 were tested for their performance. Specifically, the IVL characteristics of the devices were tested under the condition of 10 mA/cm², and the T95 lifetime of the devices was tested under the condition of 20 mA/cm². Test results are shown in Table 13.

TABLE 13

| | Light emitting layer | | | | | |
|---|---|---|---|---|---|---|
| Examples | Compound X: | Operating | | | | T95(hrs) |
| NO. | RH-N:RD-1 | Voltage (V) | Cd/A | CIEx | CIEy | @20 mA/cm² |
| Example 1 | Compound A-3 | 3.45 | 61.9 | 0.680 | 0.320 | 529 |
| Example 2 | Compound A-8 | 3.42 | 61.4 | 0.680 | 0.320 | 539 |
| Example 3 | Compound A-14 | 3.46 | 61.2 | 0.680 | 0.320 | 516 |
| Example 4 | Compound A-17 | 3.46 | 61.8 | 0.680 | 0.320 | 518 |
| Example 5 | Compound A-19 | 3.46 | 61.3 | 0.680 | 0.320 | 538 |
| Example 6 | Compound A-22 | 3.45 | 60.8 | 0.680 | 0.320 | 525 |
| Example 7 | Compound A-27 | 3.45 | 61.2 | 0.680 | 0.320 | 540 |
| Example 8 | Compound A-29 | 3.45 | 61.5 | 0.680 | 0.320 | 538 |
| Example 9 | Compound A-32 | 3.43 | 60.5 | 0.680 | 0.320 | 528 |
| Example 10 | Compound A-34 | 3.45 | 60.3 | 0.680 | 0.320 | 522 |
| Example 11 | Compound A-43 | 3.44 | 60.6 | 0.680 | 0.320 | 536 |
| Example 12 | Compound A-42 | 3.44 | 60.3 | 0.680 | 0.320 | 526 |
| Example 13 | Compound A-75 | 3.46 | 61.3 | 0.680 | 0.320 | 518 |
| Example 14 | Compound A-86 | 3.45 | 61.7 | 0.680 | 0.320 | 525 |
| Example 15 | Compound A-87 | 3.47 | 60.5 | 0.680 | 0.320 | 534 |
| Example 16 | Compound A-90 | 3.42 | 61.6 | 0.680 | 0.320 | 513 |
| Example 17 | Compound A-94 | 3.44 | 60.5 | 0.680 | 0.320 | 519 |
| Example 18 | Compound A-110 | 3.42 | 60.2 | 0.680 | 0.320 | 533 |
| Example 19 | Compound A-119 | 3.47 | 60.3 | 0.680 | 0.320 | 520 |
| Example 20 | Compound A-129 | 3.42 | 61.6 | 0.680 | 0.320 | 528 |
| Example 21 | Compound A-150 | 3.42 | 62.1 | 0.680 | 0.320 | 525 |
| Example 22 | Compound A-192 | 3.46 | 60.9 | 0.680 | 0.320 | 530 |
| Example 23 | Compound A-196 | 3.47 | 61.8 | 0.680 | 0.320 | 532 |
| Example 24 | Compound A-199 | 3.43 | 61.0 | 0.680 | 0.320 | 521 |
| Example 25 | Compound A-205 | 3.47 | 61.7 | 0.680 | 0.320 | 523 |
| Example 26 | Compound A-227 | 3.46 | 60.5 | 0.680 | 0.320 | 524 |
| Example 27 | Compound A-242 | 3.42 | 60.5 | 0.680 | 0.320 | 531 |
| Example 28 | Compound A-262 | 3.47 | 61.8 | 0.680 | 0.320 | 528 |
| Example 29 | Compound A-269 | 3.43 | 60.3 | 0.680 | 0.320 | 527 |
| Example 30 | Compound A-282 | 3.46 | 56.9 | 0.680 | 0.320 | 523 |
| Example 31 | Compound A-287 | 3.45 | 56.8 | 0.680 | 0.320 | 520 |
| Example 32 | Compound A-321 | 3.44 | 57.0 | 0.680 | 0.320 | 530 |
| Example 33 | Compound A-324 | 3.45 | 61.4 | 0.680 | 0.320 | 522 |
| Example 34 | Compound A-325 | 3.47 | 57.3 | 0.680 | 0.320 | 529 |
| Example 35 | Compound A-326 | 3.42 | 57.1 | 0.680 | 0.320 | 538 |
| Example 36 | Compound A-328 | 3.42 | 57.2 | 0.680 | 0.320 | 536 |
| Example 37 | Compound A-329 | 3.47 | 57.1 | 0.680 | 0.320 | 523 |
| Example 38 | Compound A-348 | 3.44 | 57.5 | 0.680 | 0.320 | 480 |
| Example 39 | Compound A-350 | 3.42 | 57.3 | 0.680 | 0.320 | 483 |
| Example 40 | Compound A-349 | 3.47 | 57.4 | 0.680 | 0.320 | 474 |
| Example 41 | Compound A-369 | 3.44 | 57.5 | 0.680 | 0.320 | 478 |
| Example 42 | Compound A-370 | 3.45 | 58.0 | 0.680 | 0.320 | 480 |
| Example 43 | Compound A-371 | 3.44 | 57.9 | 0.680 | 0.320 | 481 |
| Example 44 | Compound A-409 | 3.45 | 57.5 | 0.680 | 0.320 | 471 |
| Example 45 | Compound A-410 | 3.45 | 57.8 | 0.680 | 0.320 | 482 |
| Comparative | Compound A | 3.46 | 51.3 | 0.680 | 0.320 | 425 |
| Example 1 | | | | | | |
| Comparative | Compound B | 3.48 | 47.8 | 0.680 | 0.320 | 408 |
| Example 2 | | | | | | |
| Comparative | Compound C | 3.49 | 46.6 | 0.680 | 0.320 | 403 |
| Example 3 | | | | | | |

As can be seen from Table 13 above, the use of the compounds of the present application as a hole transport-type host material in a mixed-type red light-emitting host material improves the efficiency by at least 10.7%, and prolongs the service life by at least 10.8%.

### Example 46: Red Light-emitting Organic Electroluminescent Devices

First, anode pretreatment was performed in the following process. A surface of an ITO/Ag/ITO substrate, with thicknesses of ITO/Ag/ITO being 100 Å, 1000 Å, and 100 Å, respectively, was treated using ultraviolet ozone and O₂:N₂ plasma to increase the work function of the anode, and then cleaned with an organic solvent to remove impurities and oil on the ITO substrate.

Compound PD was deposited by vacuum evaporation on the experimental substrate (anode) to form a hole injection layer (HIL) having a thickness of 100 Å, and then Compound HT-1 was deposited by vacuum evaporation on the hole injection layer to form a first hole transport layer having a thickness of 1040 Å.

Compound HT-2 was deposited by vacuum evaporation on the first hole transport layer to form a second hole transport layer having a thickness of 860 Å.

Next, Compound B-49 and Compound RD-1 were co-deposited by evaporation on the second hole transport layer at an evaporation rate ratio of 98%:2% to form a red light light emitting layer (EML) having a thickness of 410 Å.

Compound ET-1 and Compound LiQ were mixed at a 1:1 weight ratio and deposited by evaporation on the light emitting layer to form an electron transport layer (ETL) having a thickness of 350 Å; Yb was deposited by evaporation on the electron transport layer to form an electron injection layer (EIL) having a thickness of 10 Å; and then magnesium (Mg) and silver (Ag) were mixed at an evaporation rate ratio of 1:9, and deposited by vacuum evaporation on the electron injection layer to form a cathode having a thickness of 130 Å.

Further, Compound CPL-1 was deposited by vacuum evaporation on the above cathode to form a capping layer having a thickness of 800 Å, completing the fabrication of a red-light organic electroluminescent device.

### Example 47-69

Organic electroluminescent devices were fabricated by the same method as used in Example 46, except that Compound B-49 in Example 46 was replaced with Compound Y shown in the following Table 14 when an light emitting layer was formed.

### Comparative Examples 4-5

Organic electroluminescent devices were fabricated by the same method as used in Example 46, except that Compound B-49 in Example 46 was replaced with a respective one of Compound D and Compound E when an light emitting layer was formed.

In Examples 46-69 and Comparative Examples 4-5, structures of the compounds used are as follows:

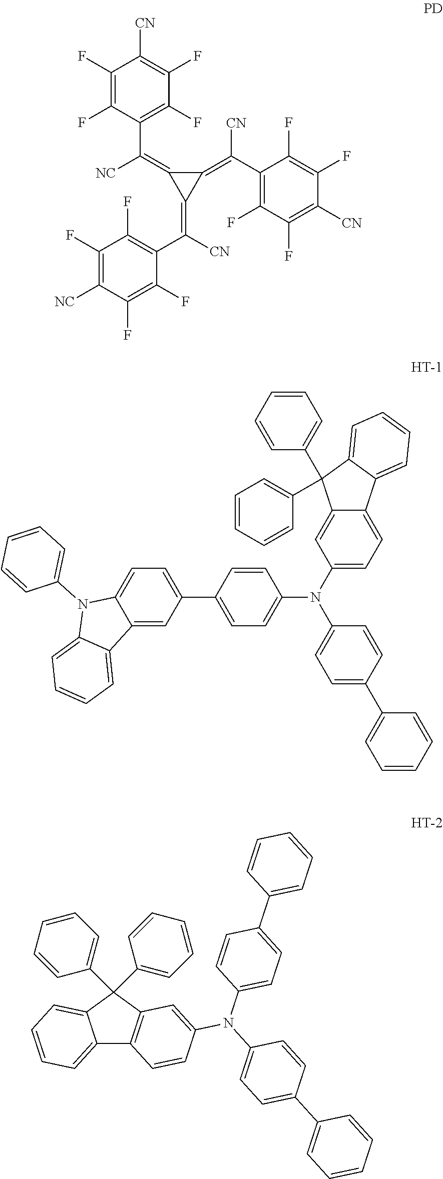 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(-c2ccc(N(c3ccc(-c4ccccc4)cc3)c3ccc4c(c3)C(c3ccccc3)(c3ccccc3)c3ccccc3-4)cc2)cc1]

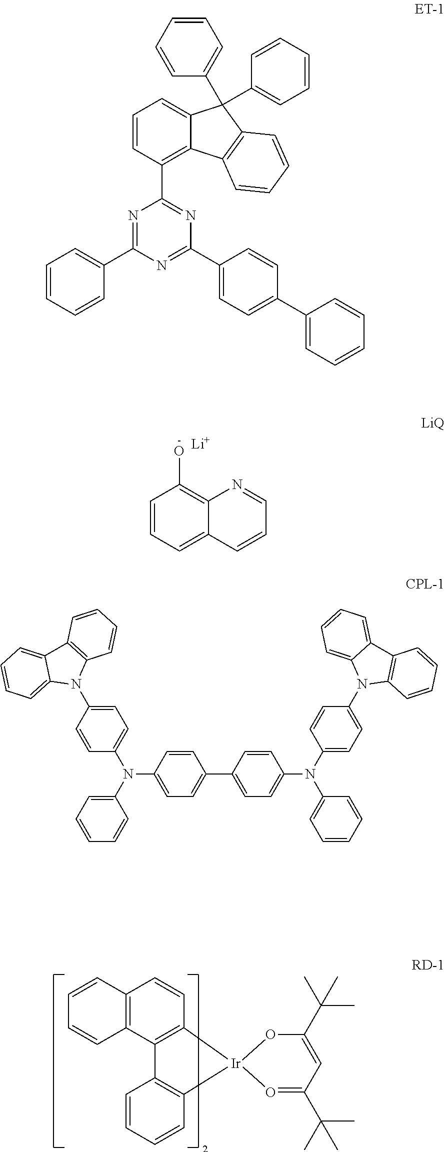

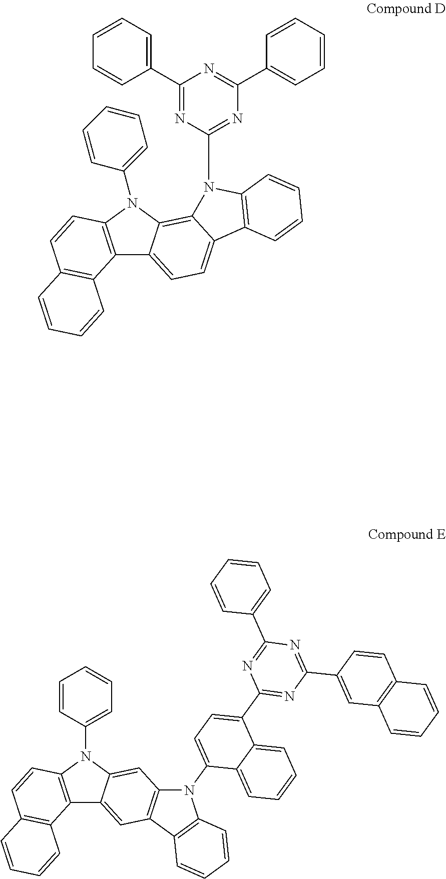 [SMILES: c1ccc(-c2nc(-c3ccccc3)nc(-n3c4ccccc4c4ccc5c6c7ccccc7ccc6n(-c6ccccc6)c5c43)n2)cc1 ; c1ccc(-c2nc(-c3ccc4ccccc4c3)nc(-c3ccc(-n4c5ccccc5c5cc6c7c8ccccc8ccc7n(-c7ccccc7)c6cc54)c4ccccc34)n2)cc1]

The red-light organic electroluminescent devices fabricated in Examples 46-69 and Comparative Examples 4-5 were tested for their performance. Specifically, the IVL characteristics of the devices were tested under the condition of 10 mA/cm², and the T95 lifetime of the devices was tested under the condition of 20 mA/cm². Test results are shown in Table 14.

TABLE 14

| | Light emitting layer | | | | | |
|---|---|---|---|---|---|---|
| Examples | Compound Y: | Operating | | | | T95(hrs)@ |
| NO. | RD-1 | Voltage (V) | Cd/A | CIEx | CIEy | 20 mA/cm² |
| Example 46 | Compound B-49 | 3.45 | 52.9 | 0.680 | 0.320 | 525 |
| Example 47 | Compound B-56 | 3.46 | 52.7 | 0.680 | 0.320 | 528 |
| Example 48 | Compound B-53 | 3.47 | 52.1 | 0.680 | 0.320 | 527 |
| Example 49 | Compound B-60 | 3.46 | 52.3 | 0.680 | 0.320 | 523 |
| Example 50 | Compound B-69 | 3.45 | 52.8 | 0.680 | 0.320 | 532 |
| Example 51 | Compound B-115 | 3.47 | 52.4 | 0.680 | 0.320 | 528 |
| Example 52 | Compound B-121 | 3.44 | 52.6 | 0.680 | 0.320 | 522 |
| Example 53 | Compound B-123 | 3.45 | 52.5 | 0.680 | 0.320 | 530 |
| Example 54 | Compound B-142 | 3.47 | 56.1 | 0.680 | 0.320 | 552 |
| Example 55 | Compound B-146 | 3.47 | 56.2 | 0.680 | 0.320 | 558 |
| Example 56 | Compound B-155 | 3.43 | 56.7 | 0.680 | 0.320 | 565 |
| Example 57 | Compound B-157 | 3.43 | 56.2 | 0.680 | 0.320 | 567 |
| Example 58 | Compound B-159 | 3.47 | 57.1 | 0.680 | 0.320 | 557 |
| Example 59 | Compound B-168 | 3.47 | 56.0 | 0.680 | 0.320 | 552 |
| Example 60 | Compound B-172 | 3.47 | 56.3 | 0.680 | 0.320 | 574 |
| Example 61 | Compound B-173 | 3.43 | 56.5 | 0.680 | 0.320 | 564 |
| Example 62 | Compound B-182 | 3.45 | 56.3 | 0.680 | 0.320 | 566 |
| Example 63 | Compound B-186 | 3.42 | 57.5 | 0.680 | 0.320 | 556 |
| Example 64 | Compound B-189 | 3.47 | 57.1 | 0.680 | 0.320 | 567 |
| Example 65 | Compound B-193 | 3.44 | 56.2 | 0.680 | 0.320 | 559 |
| Example 66 | Compound B-219 | 3.42 | 52.0 | 0.680 | 0.320 | 561 |
| Example 67 | Compound B-244 | 3.46 | 52.2 | 0.680 | 0.320 | 552 |
| Example 68 | Compound B-255 | 3.45 | 52.7 | 0.680 | 0.320 | 574 |
| Example 69 | Compound B-269 | 3.45 | 52.2 | 0.680 | 0.320 | 550 |
| Comparative | Compound D | 3.49 | 45.2 | 0.680 | 0.320 | 468 |
| Example 4 | | | | | | |
| Comparative | Compound E | 3.51 | 44.3 | 0.680 | 0.320 | 472 |
| Example 5 | | | | | | |

As can be seen from Table 14 above, when the compounds of the present application are used as a bipolar red light-emitting host material, the efficiency of the devices is improved by at least 15.0%, and the service life thereof is prolonged by at least 10.6%.

### Example 70: Green Light-emitting Organic Electroluminescent Devices

First, anode pretreatment was performed in the following process. A surface of an ITO/Ag/ITO substrate, with thicknesses of ITO/Ag/ITO being 100 Å, 1000 Å, and 100 Å, respectively, was treated using ultraviolet ozone and O₂:N₂ plasma to increase the work function of the anode, and then cleaned with an organic solvent to remove impurities and oil on the ITO substrate.

Compound PD was deposited by vacuum evaporation on the experimental substrate (anode) to form a hole injection layer (HIL) having a thickness of 100 Å, and then NPB was deposited by evaporation on the hole injection layer to form a first hole transport layer having a thickness of 930 Å.

Compound HT-15 was deposited by vacuum evaporation on the first hole transport layer to form a second hole transport layer having a thickness of 760 Å.

Compound B-3, Compound GH-P, and Compound GD were co-deposited by evaporation on the second hole transport layer at an evaporation rate ratio of 40%:55%:5% to form a green light-emitting light emitting layer (EML) having a thickness of 340 Å.

Compound ET-6 and Compound LiQ were mixed at a 1:1 weight ratio and deposited by evaporation to form an electron transport layer (ETL) having a thickness of 300 Å; Yb was deposited by evaporation on the electron transport layer to form an electron injection layer (EIL) having a thickness of 15 Å; and then magnesium (Mg) and silver (Ag) were mixed at an evaporation rate ratio of 1:9, and deposited by vacuum evaporation on the electron injection layer to form a cathode having a thickness of 120 Å.

Further, Compound CPL-1 was deposited by vacuum evaporation on the above cathode to form a capping layer having a thickness of 650 Å, completing the fabrication of an organic electroluminescent device. The device fabricated was marked as Example 70.

### Examples 71-80

Organic electroluminescent devices were fabricated by the same method as used in Example 70, except that Compound B-3 in Example 70 was replaced with Compound Z shown in the following Table 15 when an light emitting layer was formed.

### Comparative Examples 6-7

Organic electroluminescent devices were fabricated by the same method as used in Example 70, except that Compound B-3 in Example 70 was replaced with a respective one of Compound G and Compound F when an light emitting layer was formed.

In the fabrication of various examples and comparative examples, structures of the compounds used are as follows:

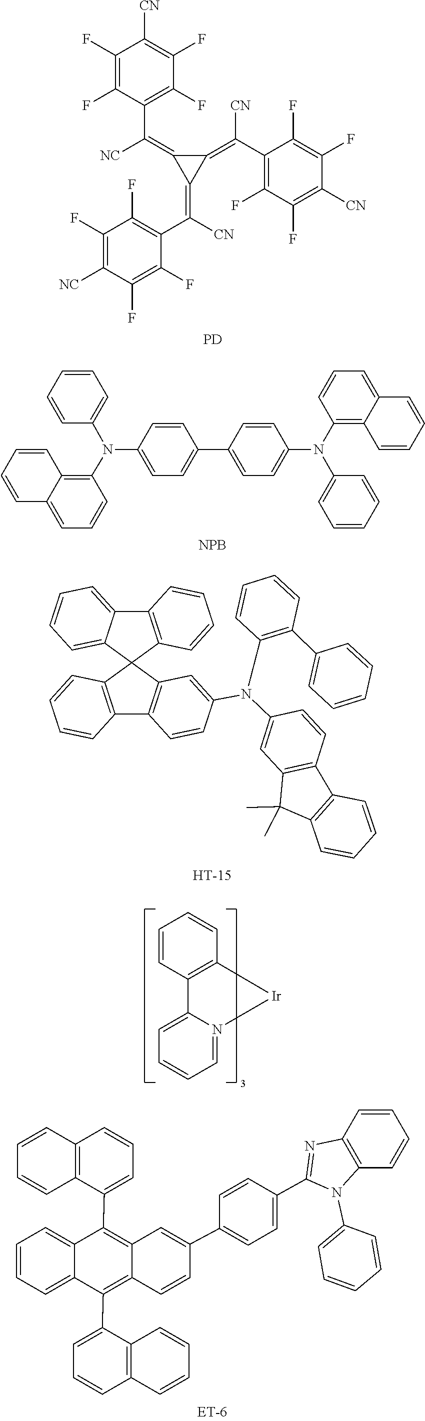

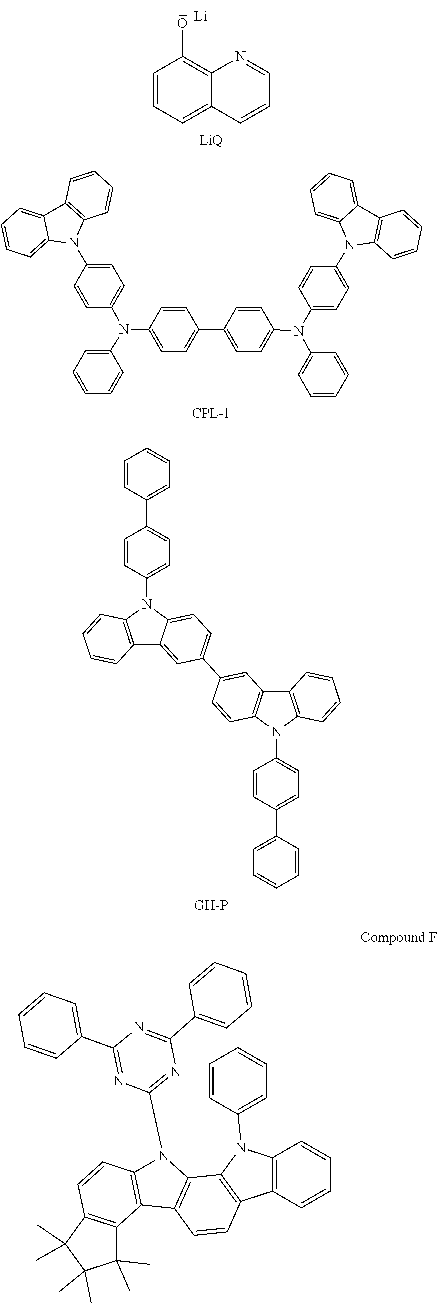

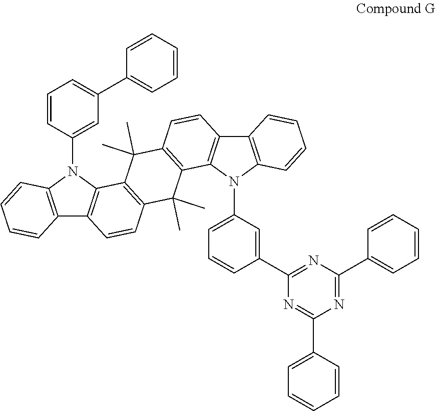 [SMILES: CC1(C)c2ccc3c4ccccc4n(-c4cccc(-c5nc(-c6ccccc6)nc(-c6ccccc6)n5)c4)c3c2C(C)(C)c2ccc3c4ccccc4n(-c4cccc(-c5ccccc5)c4)c3c21]

The green-light organic electroluminescent devices fabricated in Examples 70-80 and Comparative Examples 6-7 were tested for their performance. Specifically, the IVL characteristics of the devices were tested under the condition of 10 mA/cm², and the T95 lifetime of the devices was tested under the condition of 20 mA/cm². Test results are shown in Table 15.

TABLE 15

| | Light emitting layer | | | | | |
|---|---|---|---|---|---|---|
| Examples | Compound Z: | Operating | | | | T95(hrs) |
| NO. | GH-P:GD | Voltage (V) | Cd/A | CIEx | CIEy | @20 mA/cm² |
| Example 70 | Compound B-3 | 3.87 | 87.7 | 0.250 | 0.680 | 295 |
| Example 71 | Compound B-7 | 3.85 | 87.3 | 0.250 | 0.680 | 279 |
| Example 72 | Compound B-32 | 3.87 | 88.0 | 0.250 | 0.680 | 288 |
| Example 73 | Compound B-36 | 3.87 | 87.9 | 0.250 | 0.680 | 291 |
| Example 74 | Compound B-39 | 3.84 | 87.0 | 0.250 | 0.680 | 274 |
| Example 75 | Compound B-46 | 3.87 | 86.6 | 0.250 | 0.680 | 287 |
| Example 76 | Compound B-47 | 3.84 | 86.1 | 0.250 | 0.680 | 286 |
| Example 77 | Compound B-83 | 3.84 | 87.8 | 0.250 | 0.680 | 287 |
| Example 78 | Compound B-98 | 3.87 | 86.1 | 0.250 | 0.680 | 279 |
| Example 79 | Compound B-105 | 3.83 | 88.1 | 0.250 | 0.680 | 277 |
| Example 80 | Compound B-112 | 3.87 | 87.0 | 0.250 | 0.680 | 294 |
| Comparative | Compound F | 3.88 | 74.1 | 0.250 | 0.680 | 240 |
| Example 6 | | | | | | |
| Comparative | Compound G | 3.89 | 73.4 | 0.250 | 0.680 | 235 |
| Example 7 | | | | | | |

As can be seen from Table 15 above, when the compounds of the present application are used as an electron transport-type host material in a mixed-type green light-emitting host material, the efficiency of the devices is improved by at least 16.2%, and the service life thereof is prolonged by at least 14.2%.

The reason is that structures of the compounds of the present application each include a core structure that is tetramethylcyclohexane-fused indolocarbazole, which is further linked with an aryl or heteroaryl group via two nitrogen atoms in indolocarbazole, and linked with an electron-deficient heteroarylene group containing at least two nitrogen atoms. The indolocarbazolyl group has an excellent hole transport ability, and the structure of the substituted cyclohexane can further enhance the hole transport ability of the carbazolyl group as a result of the hyperconjugation effect, thereby giving the compounds of the present application an excellent hole transport ability. When tetramethylcyclohexane-fused indolocarbazole is linked with an electron-rich aryl or heteroaryl group, it can form a material with an excellent hole transport property; and when tetramethylcyclohexane-fused indolocarbazole is linked with an electron-deficient heteroarylene group containing at least two nitrogen atoms, it can form a material with an electron transport property or a bipolar material. In addition, the four methyl groups, in spatial configuration, are outside the conjugate plane of the carbazolyl group, and thus form a certain degree of steric hindrance, which can result in fine regulation of intermolecular accumulation in the compounds, and make it possible to form the compounds into better amorphous thin films. The use of the compounds of the present application as a host material of an light emitting layer can improve balance of charge carriers in the light emitting layer, expand a region where the carriers recombine, improve generation and utilization efficiency of excitons as well as luminescence efficiency and service life of the device.

The above describes preferred embodiments of the present application in detail with reference to the accompanying drawings. The present application, however, is not limited to the specific details in the above embodiments. Many simple variations can be made to the technical solutions of the present application within the scope of the technical concepts of the present application, and these simple variations are within the scope of protection of the present application.

1. A nitrogen-containing compound having a structure formed by fusing a structure of the following Formula 1 and a structure of the following Formula 2 together, wherein the structure of Formula 2 is fused via * to positions of any two adjacent * on Ring B in the structure of Formula 1; 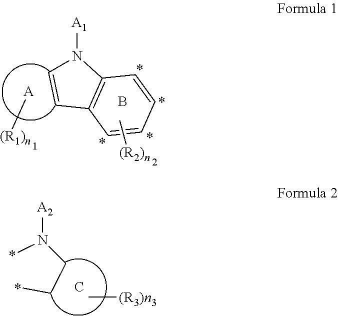
Ring A is benzocyclohexane;
Ring C is selected from an C₆₋₁₄ aromatic ring;
groups A₁ and A₂ are each independently selected from a structure shown in Formula a-1 or a structure shown in Formula a-2, and at least one of A₁ and A₂ is the structure shown in Formula a-1; 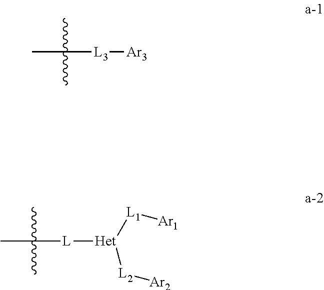
L, L₁, L₂ and L₃ are identical or different, and are each independently selected from a single bond, a substituted or unsubstituted C₆₋₃₀ arylene, or a substituted or unsubstituted C₃₋₃₀ heteroarylene;
Ar₃ is selected from a substituted or unsubstituted C₆₋₄₀ aryl, or a substituted or unsubstituted C₃₋₄₀ heteroaryl;
Het is a C₃₋₂₀ nitrogen-containing heteroarylene;
Ar₁ and Ar₂ are identical or different, and are each independently selected from hydrogen, a substituted or unsubstituted C₆₋₄₀ aryl, or a substituted or unsubstituted C₃₋₄₀ heteroaryl;
each R₁, each R₂ and each R₃ are identical or different, and are each independently selected from deuterium, a cyano, a halogen, an C₁₋₁₀ alkyl, a C₁₋₁₀ haloalkyl, a C₁₋₁₀ deuterated alkyl, a C₃₋₁₂ trialkylsilyl, a triphenylsilyl, an C₆₋₂₀ aryl, a C₆₋₂₀ deuterated aryl, a C₆₋₂₀ halogenated aryl, a C₃₋₂₀ heteroaryl or a C₃₋₁₀ cycloalkyl; and optionally, any two adjacent R₂s form a 6-membered to 14-membered aromatic ring, the 6-membered to 14-membered aromatic ring being optionally substituted by zero, one, two, three, four, five or six R₄s;
each R₄ is independently selected from deuterium, a cyano, a halogen, an C₁₋₁₀ alkyl, a C₁₋₁₀ haloalkyl, a C₁₋₁₀ deuterated alkyl, or a C₃₋₁₂ trialkylsilyl;
n₁ is selected from 1, 2, 3, 4, 5, 6, 7, 8, 9 or 10;
n₂ is selected from 0, 1 or 2;
n₃ is selected from 0, 1, 2, 3, 4, 5, 6, 7 or 8;
substituents of L, L₁, L₂, L₃, Ar₁, Ar₂ and Ar₃ are identical or different, and are each independently selected from deuterium, a cyano, a halogen, an C₁₋₁₀ alkyl, a C₁₋₁₀ haloalkyl, a C₁₋₁₀ deuterated alkyl, a C₃₋₁₂ trialkylsilyl, a triphenylsilyl, an C₆₋₂₀ aryl, a C₆₋₂₀ deuterated aryl, a C₆₋₂₀ halogenated aryl, a C₃₋₂₀ heteroaryl or a C₃₋₁₀ cycloalkyl; and optionally, any two adjacent substituents may form a saturated or unsaturated 3-membered to 15-membered ring.

2. The nitrogen-containing compound according to claim 1, wherein the structure of Formula 1 is selected from structures shown in the following Formulae (i-1)-(i-3):

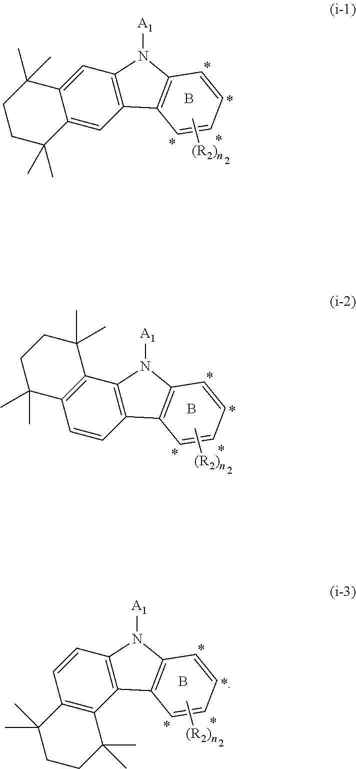

3. The nitrogen-containing compound according to claim 1, wherein Ring C is selected from a benzene ring, a naphthalene ring, or a phenanthrene ring.

4. The nitrogen-containing compound according to claim 1, wherein each R₁, each R₂ and each R₃ are identical or different, and are each independently selected from deuterium, a cyano, a fluorine, a trideuteromethyl, a trimethylsilyl, a trifluoromethyl, a cyclopentyl, a cyclohexyl, an adamantly, a methyl, an ethyl, an isopropyl, a tert-butyl, a phenyl or a naphthyl;
optionally, any two adjacent R₂s form a benzene ring, the benzene ring being substituted by zero, one, two, three, four, five or six R₄s; and each R₄ is each independently selected from deuterium, a cyano, a fluorine, a trideuteromethyl or a trifluoromethyl.

5. The nitrogen-containing compound according to claim 1, wherein Het is selected from the following groups: 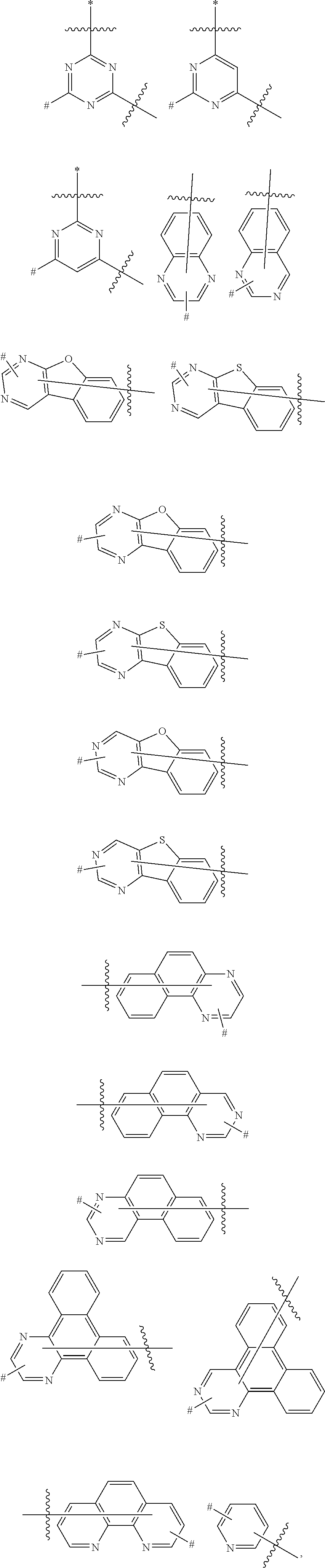
wherein [symbol] represents a bond linked with L; [symbol] represents a bond linked with L₁; [symbol] represents a bond linked with L₂; where a formula contains no [symbol] it means that, in 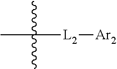
linked at this position, L₂ is a single bond and Ar₂ is hydrogen.

6. The nitrogen-containing compound according to claim 1, wherein Het is selected from the following groups: 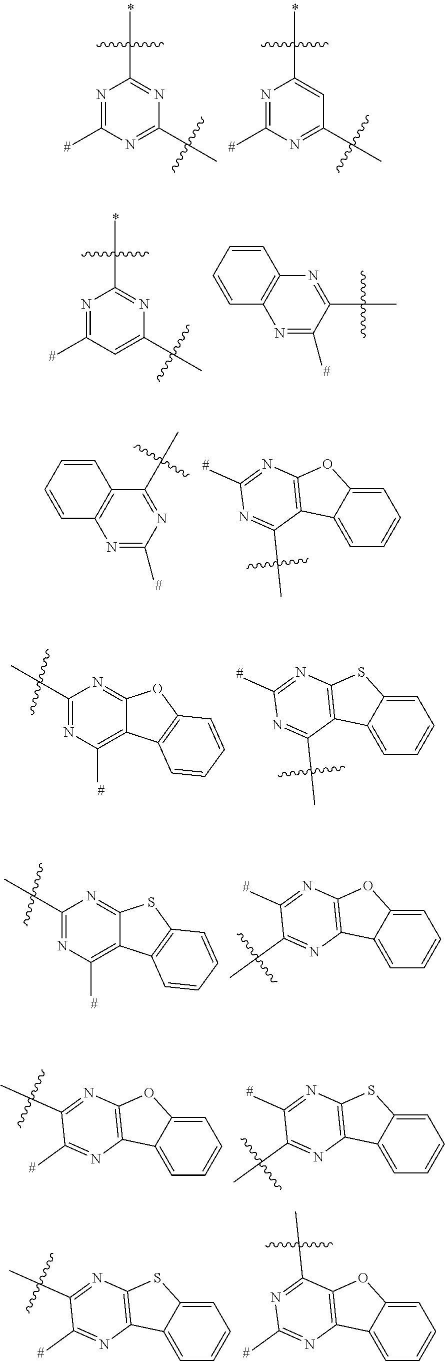 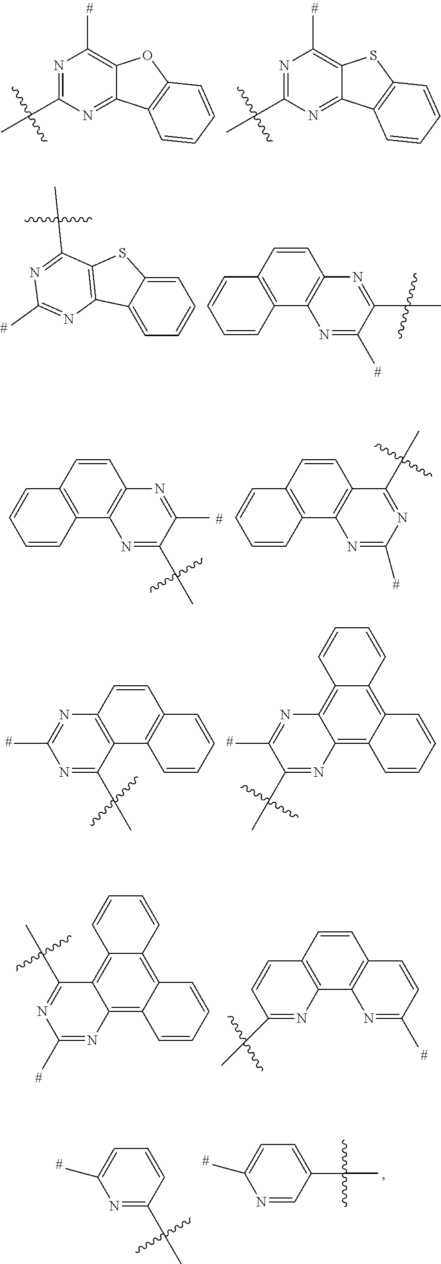
wherein [symbol] represents a bond linked with L; [symbol] represents a bond linked with L₁; [symbol] represents a bond linked with L₂; where a formula contains no [symbol] it means that, in 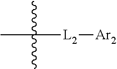
linked at this position, L₂ is a single bond and Ar₂ is hydrogen.

7. The nitrogen-containing compound according to claim 1, wherein L, L₁, L₂ and L₃ are identical or different, and are each independently selected from a single bond, a substituted or unsubstituted phenylene, a substituted or unsubstituted naphthylene, a substituted or unsubstituted biphenylene, a substituted or unsubstituted anthrylene, a substituted or unsubstituted phenanthrylene, a substituted or unsubstituted fluorenylidene, a substituted or unsubstituted pyridylidene, a substituted or unsubstituted dibenzothiophenylene, a substituted or unsubstituted dibenzofuranylene, or a substituted or unsubstituted carbazolylene;
alternatively, substituents of L, L₁, L₂ and L₃ are identical or different, and are each independently selected from deuterium, a fluorine, a cyano, a methyl, an ethyl, an isopropyl, a tert-butyl, a trifluoromethyl, a trideuteromethyl, a trimethylsilyl or a phenyl.

8. The nitrogen-containing compound according to claim 1, wherein Ar₁ and Ar₃ are each independently selected from a substituted or unsubstituted C₆₋₂₅ aryl or a substituted or unsubstituted C₇₋₂₀ heteroaryl; and Ar₂ is selected from hydrogen, a substituted or unsubstituted C₆₋₂₅ aryl, or a substituted or unsubstituted C₇₋₂₀ heteroaryl;
alternatively, substituents of Ar₁, Ar₂ and Ar₃ are each independently selected from deuterium, a halogen, a cyano, a C₁₋₄ haloalkyl, a C₁₋₄ deuterated alkyl, an C₁₋₄ alkyl, a C₅₋₁₀ cycloalkyl, an C₆₋₁₂ aryl, a C₅₋₁₂ heteroaryl or a C₃₋₈ trialkylsilyl; and optionally, any two adjacent substituents form a benzene ring or a fluorene ring.

9. The nitrogen-containing compound according to claim 1, wherein: Ar₁ and Ar₃ are each independently selected from substituted or unsubstituted groups V; and Ar₂ is selected from hydrogen and substituted or unsubstituted groups V; wherein the unsubstituted groups V are selected from the following groups: 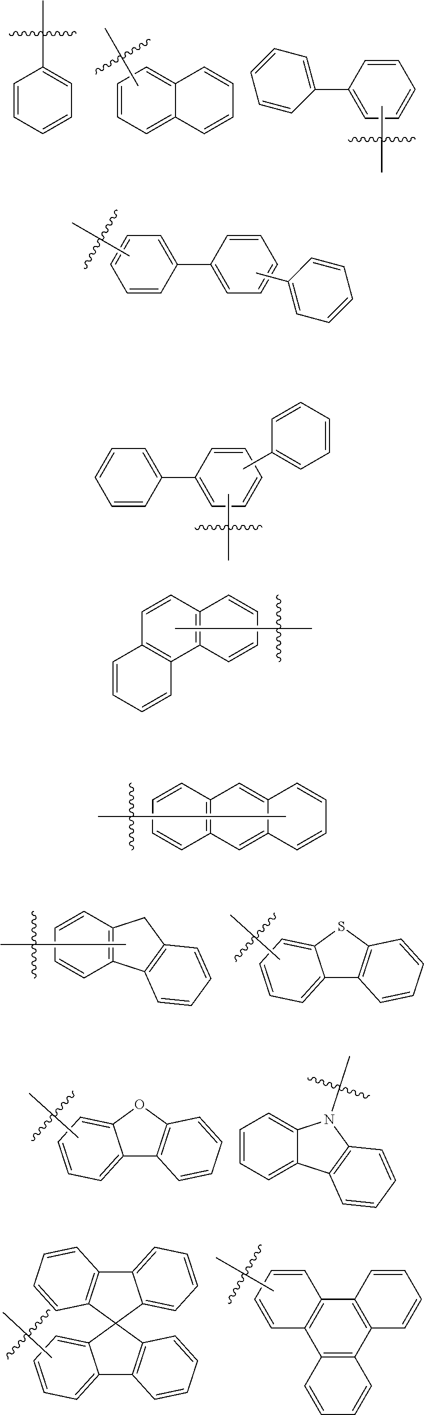 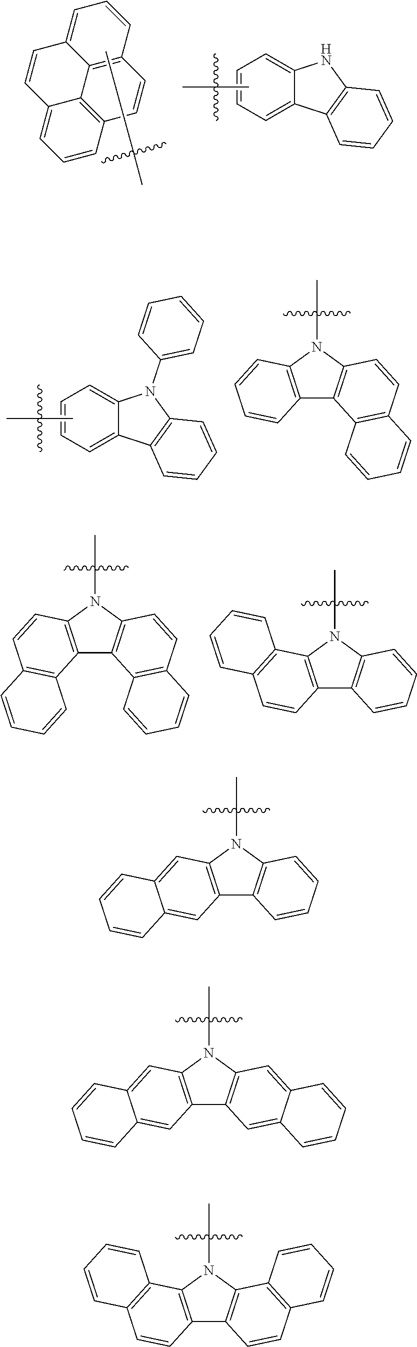 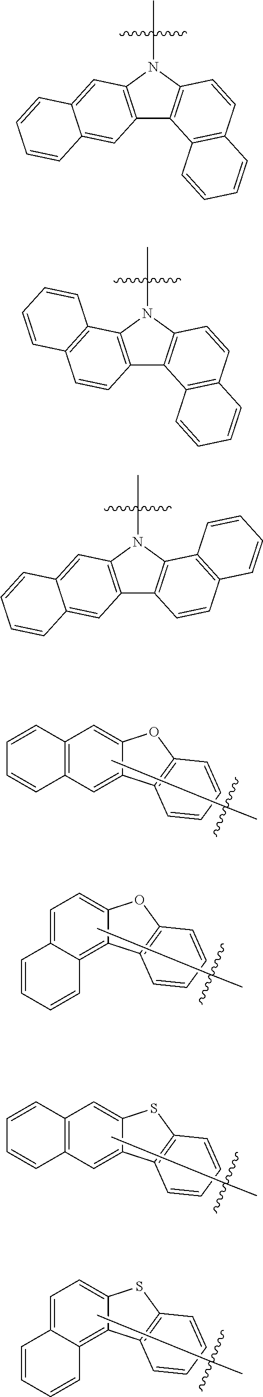 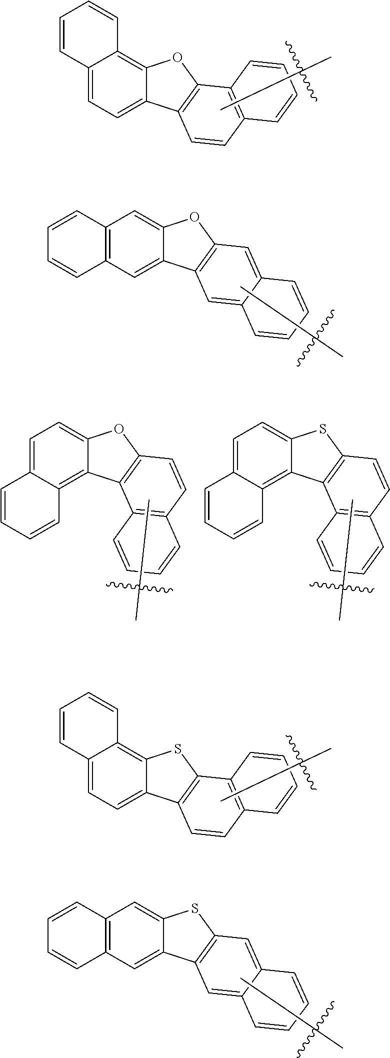 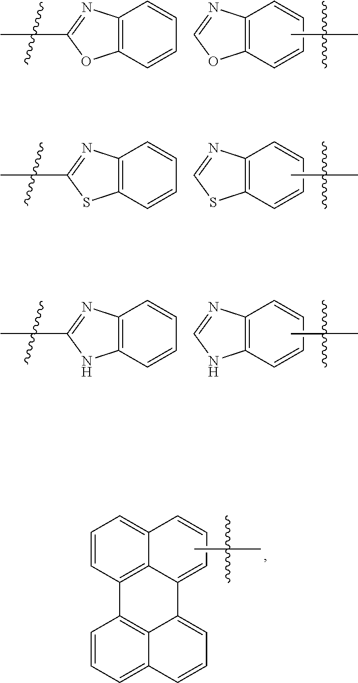
the substituted groups V each have one or more substituents, the substituents of the groups V being each independently selected from deuterium, a fluorine, a cyano, a trideuteromethyl, a trimethylsilyl, a trifluoromethyl, a cyclopentyl, a cyclohexyl, an adamantly, a methyl, an ethyl, an isopropyl, a tert-butyl, a phenyl, a naphthyl, a pyridyl, a dibenzofuranyl, a dibenzothiophenyl, a carbazolyl, a benzoxazolyl or a benzothiazolyl, and when each group V has more than one substituents, the substituents are identical or different.

10. The nitrogen-containing compound according to claim 1, wherein at least one of A₁ and A₂ is the structure shown in Formula a-1, the structure 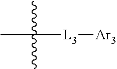
in Formula a-1 being selected from the following groups:

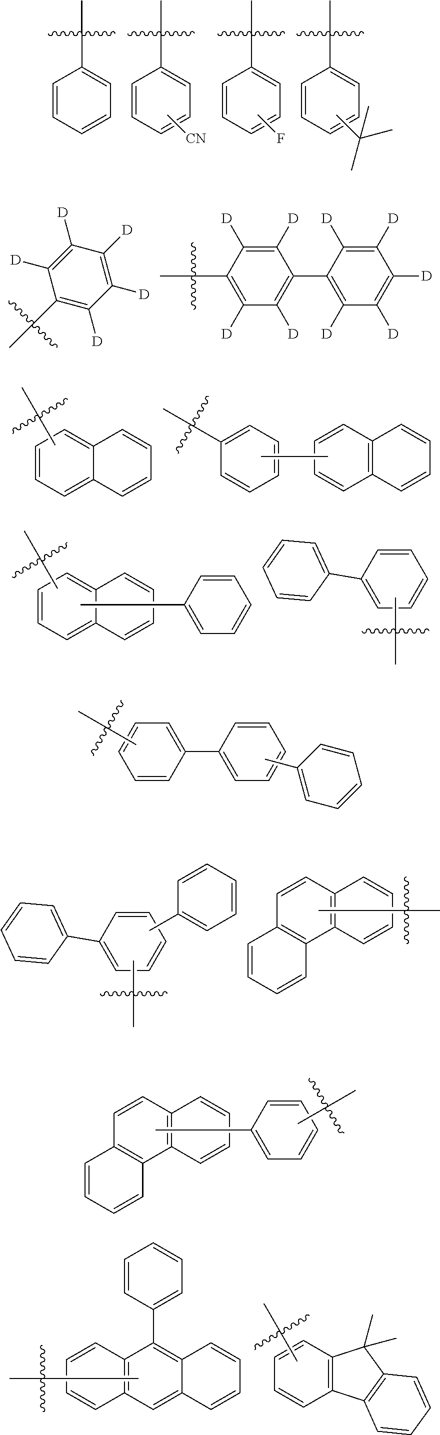

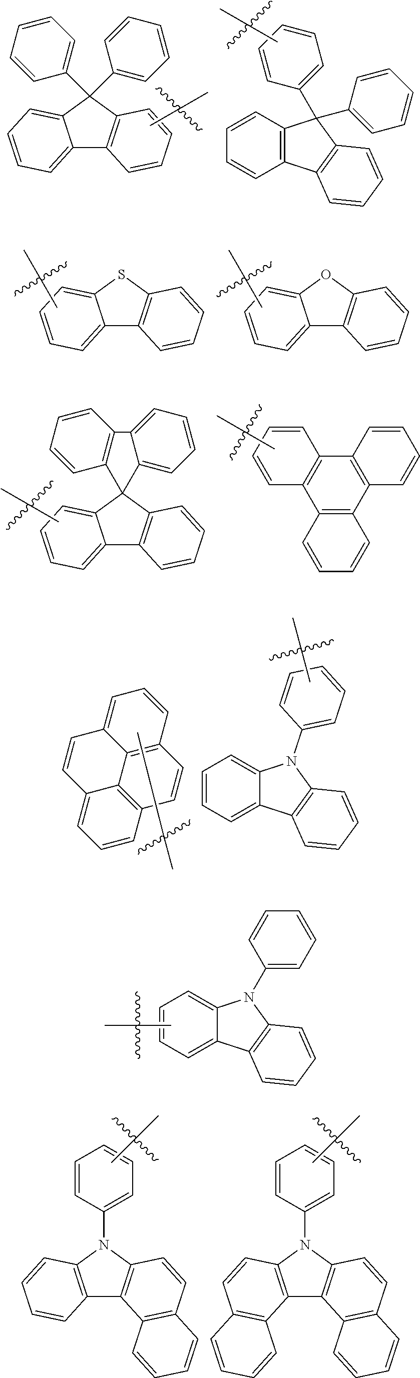

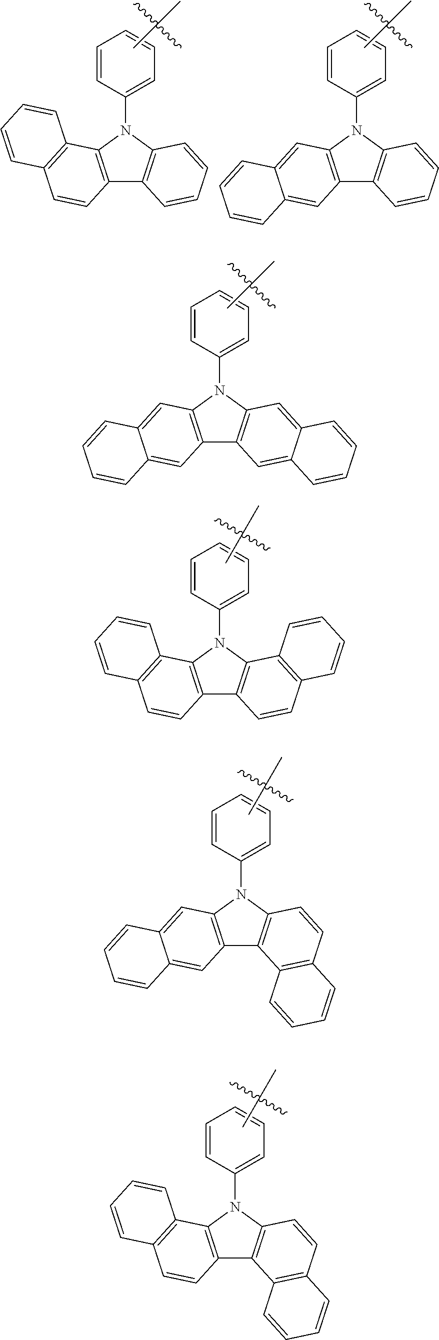

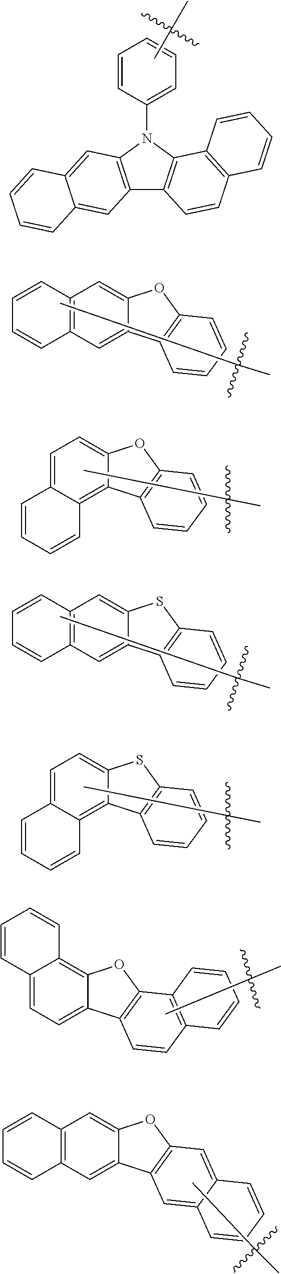

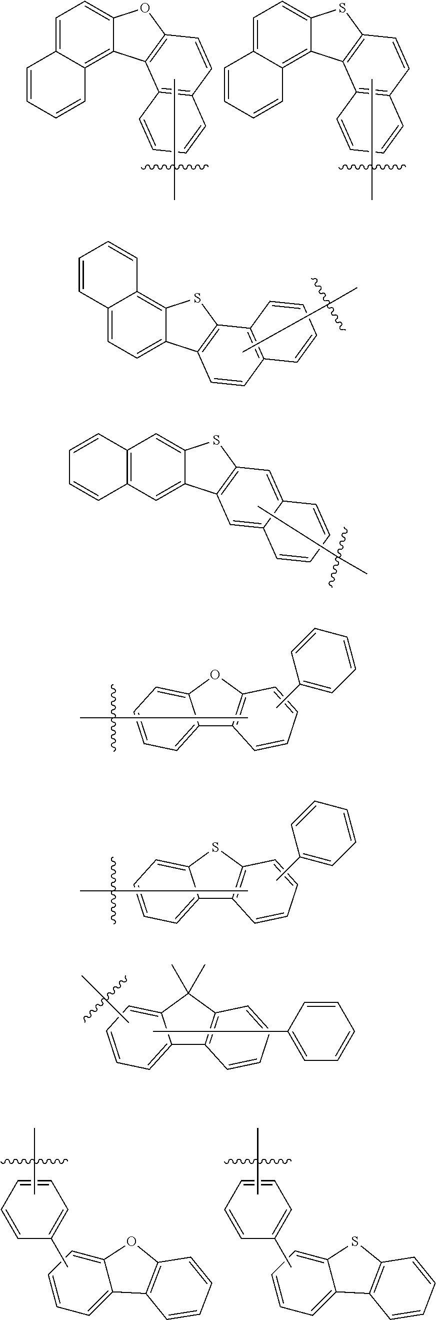

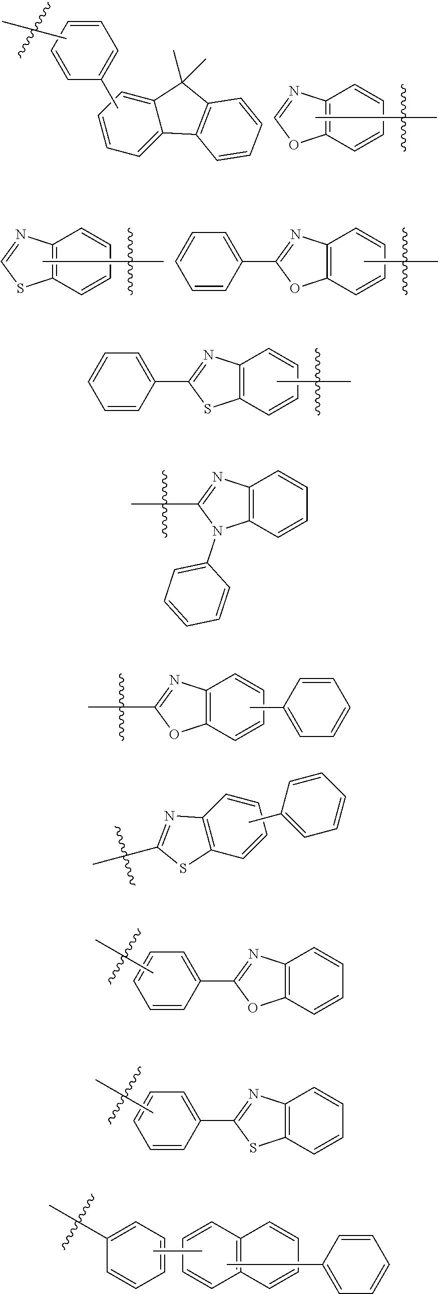

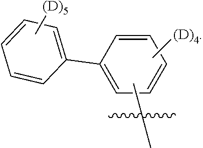

11. The nitrogen-containing compound according to claim 1, wherein 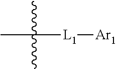
is selected from the following groups, and 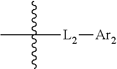
is selected from hydrogen or the following groups:

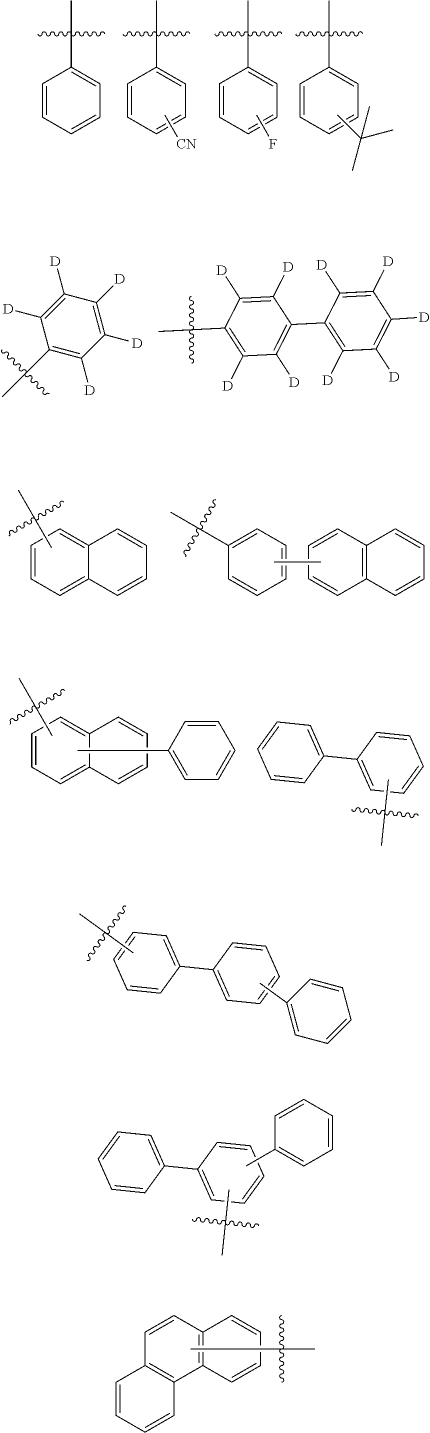

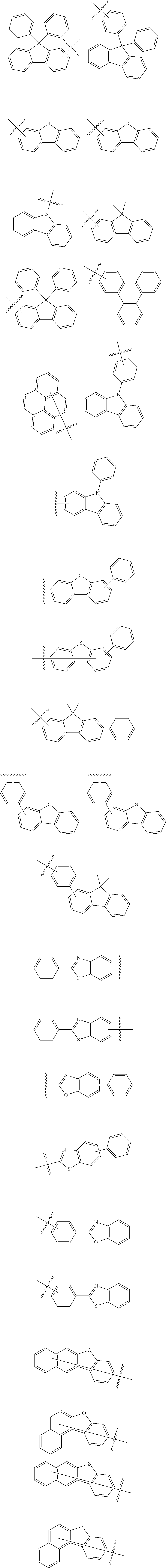

12. The nitrogen-containing compound according to claim 1, wherein the nitrogen-containing compound is selected from the group consisting of the following compounds:

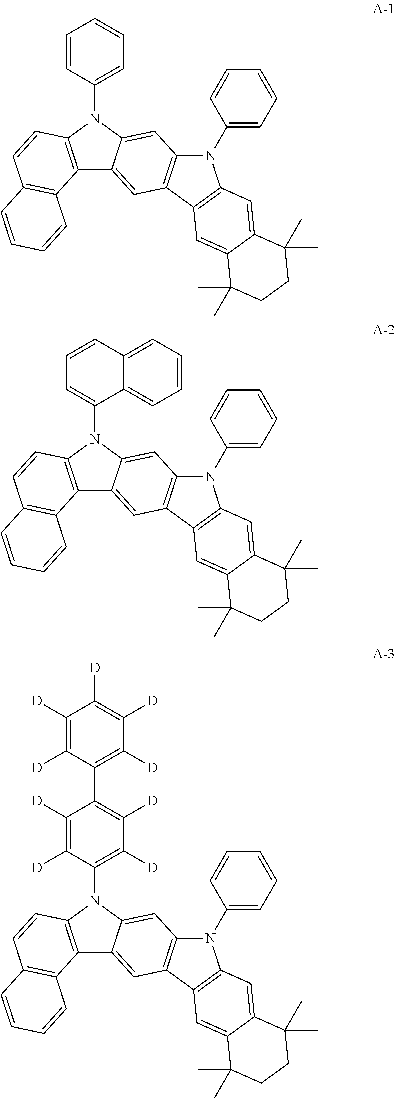 [SMILES: CC1(C)CCC(C)(C)c2cc3c(cc21)c1cc2c4c5ccccc5ccc4n(-c4ccccc4)c2cc1n3-c1ccccc1 ; CC1(C)CCC(C)(C)c2cc3c(cc21)c1cc2c4c5ccccc5ccc4n(-c4cccc5ccccc45)c2cc1n3-c1ccccc1 ; [2H]c1c([2H])c([2H])c(-c2c([2H])c([2H])c(-n3c4cc5c(cc4c4c6ccccc6ccc43)c3cc4c(cc3n5-c3ccccc3)C(C)(C)CCC4(C)C)c([2H])c2[2H])c([2H])c1[2H]]

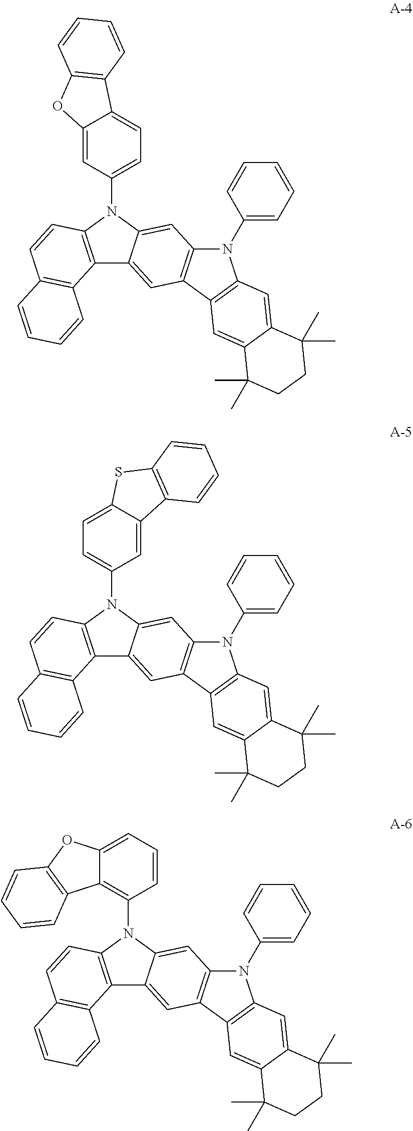

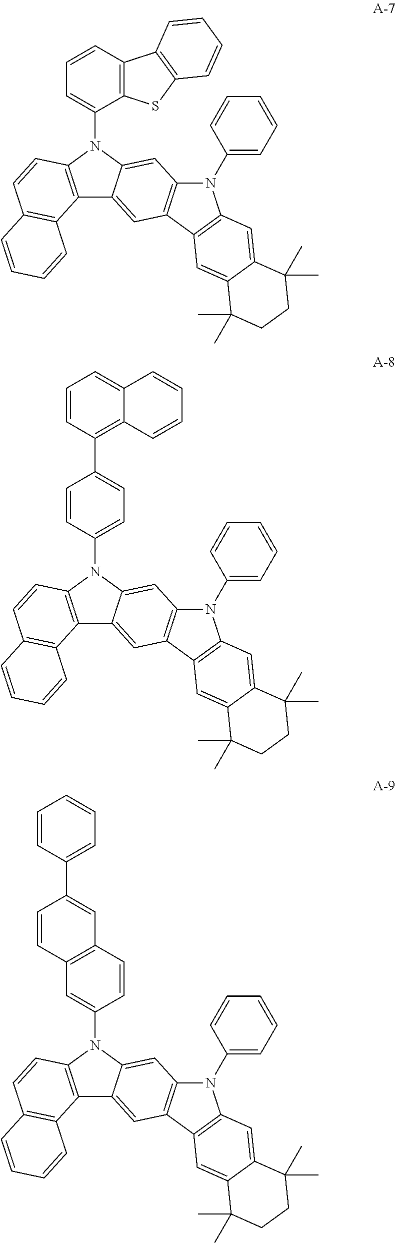 [SMILES: CC1(C)CCC(C)(C)c2cc3c(cc21)c1cc2c4c5ccccc5ccc4n(-c4cccc5c4sc4ccccc45)c2cc1n3-c1ccccc1 ; CC1(C)CCC(C)(C)c2cc3c(cc21)c1cc2c4c5ccccc5ccc4n(-c4ccc(-c5cccc6ccccc56)cc4)c2cc1n3-c1ccccc1 ; CC1(C)CCC(C)(C)c2cc3c(cc21)c1cc2c4c5ccccc5ccc4n(-c4ccc5cc(-c6ccccc6)ccc5c4)c2cc1n3-c1ccccc1]

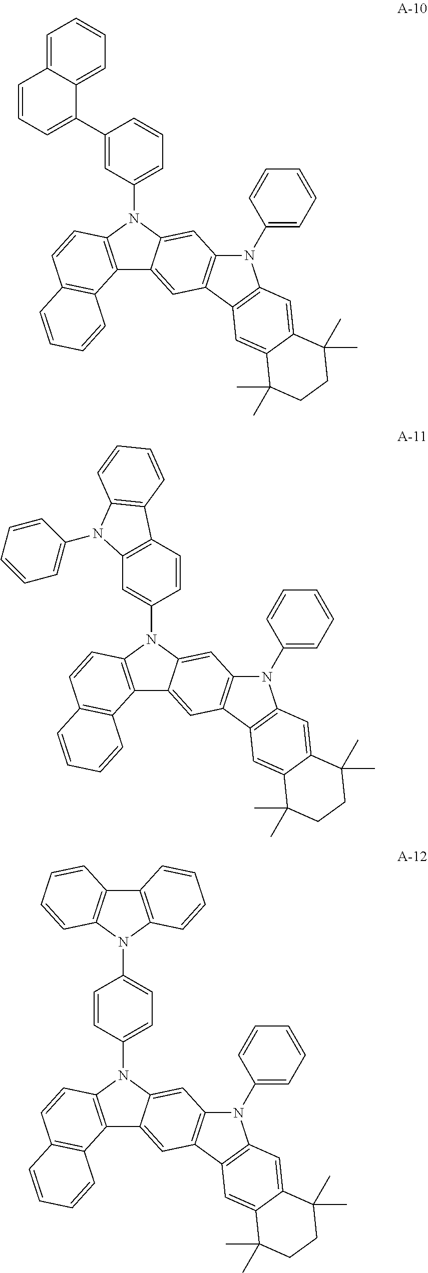 [SMILES: CC1(C)CCC(C)(C)c2cc3c(cc21)c1cc2c4c5ccccc5ccc4n(-c4cccc(-c5cccc6ccccc56)c4)c2cc1n3-c1ccccc1 ; CC1(C)CCC(C)(C)c2cc3c(cc21)c1cc2c4c5ccccc5ccc4n(-c4ccc5c6ccccc6n(-c6ccccc6)c5c4)c2cc1n3-c1ccccc1 ; CC1(C)CCC(C)(C)c2cc3c(cc21)c1cc2c4c5ccccc5ccc4n(-c4ccc(-n5c6ccccc6c6ccccc65)cc4)c2cc1n3-c1ccccc1]

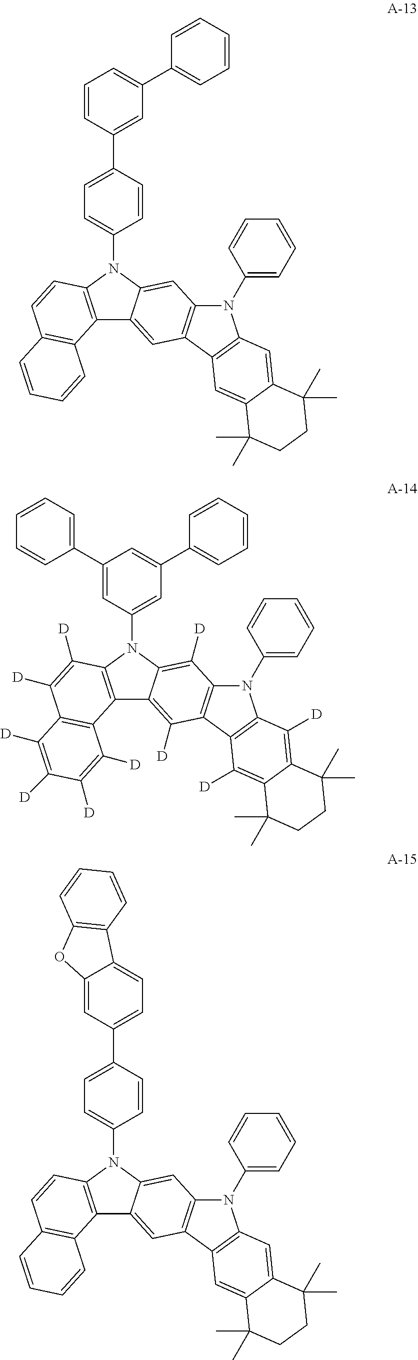

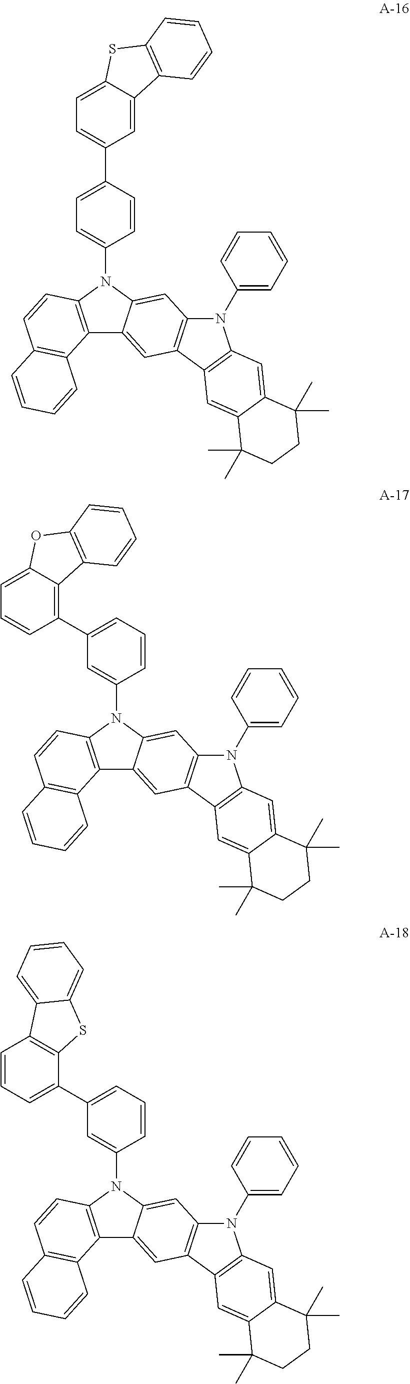 [SMILES: CC1(C)CCC(C)(C)c2cc3c(cc21)c1cc2c4c5ccccc5ccc4n(-c4ccc(-c5ccc6sc7ccccc7c6c5)cc4)c2cc1n3-c1ccccc1 ; CC1(C)CCC(C)(C)c2cc3c(cc21)c1cc2c4c5ccccc5ccc4n(-c4cccc(-c5cccc6oc7ccccc7c56)c4)c2cc1n3-c1ccccc1 ; CC1(C)CCC(C)(C)c2cc3c(cc21)c1cc2c4c5ccccc5ccc4n(-c4cccc(-c5cccc6c5sc5ccccc56)c4)c2cc1n3-c1ccccc1]

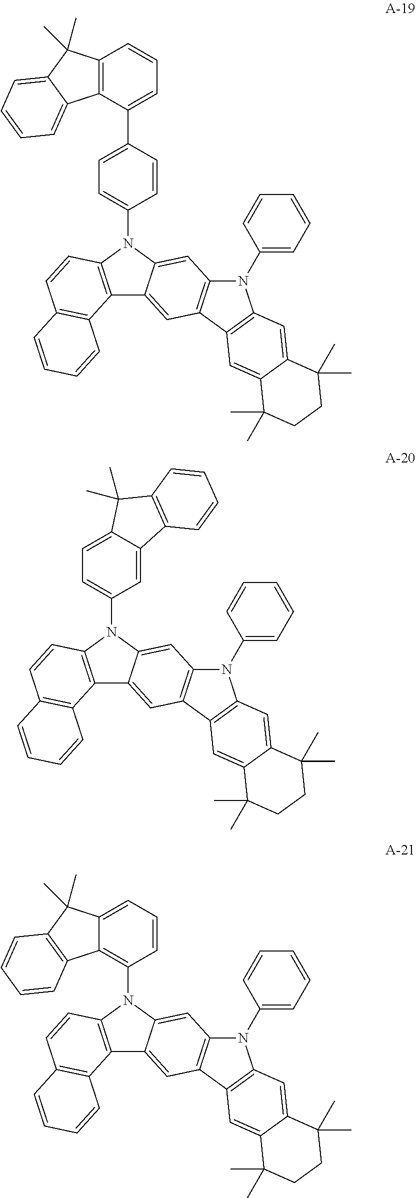 [SMILES: CC1(C)CCC(C)(C)c2cc3c(cc21)c1cc2c4c5ccccc5ccc4n(-c4ccc(-c5cccc6c5-c5ccccc5C6(C)C)cc4)c2cc1n3-c1ccccc1 ; CC1(C)CCC(C)(C)c2cc3c(cc21)c1cc2c4c5ccccc5ccc4n(-c4ccc5c(c4)-c4ccccc4C5(C)C)c2cc1n3-c1ccccc1 ; CC1(C)CCC(C)(C)c2cc3c(cc21)c1cc2c4c5ccccc5ccc4n(-c4cccc5c4-c4ccccc4C5(C)C)c2cc1n3-c1ccccc1]

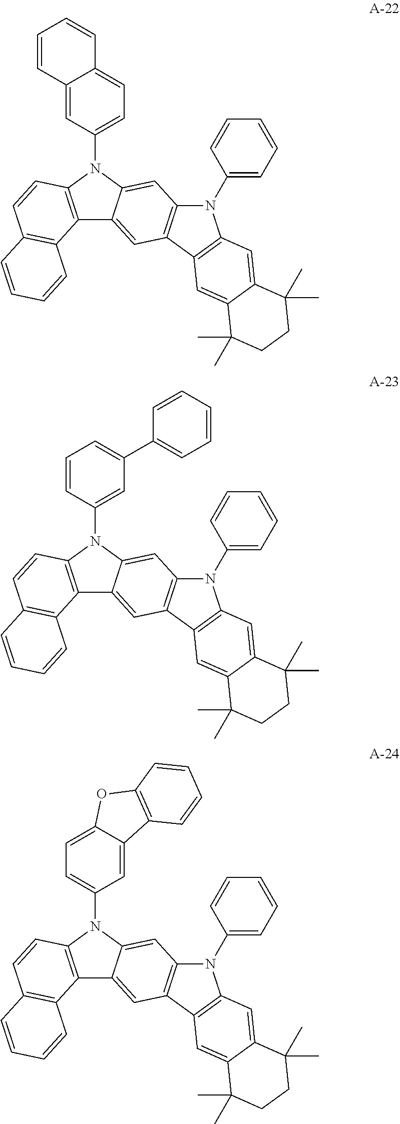

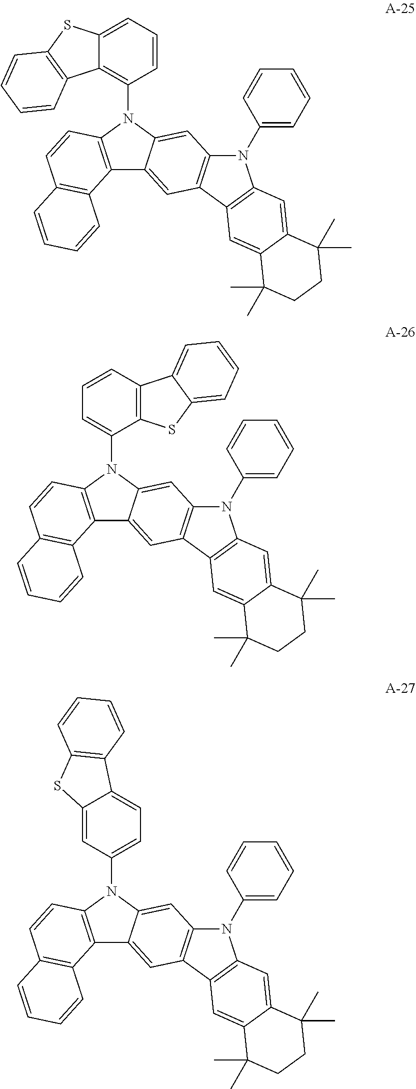

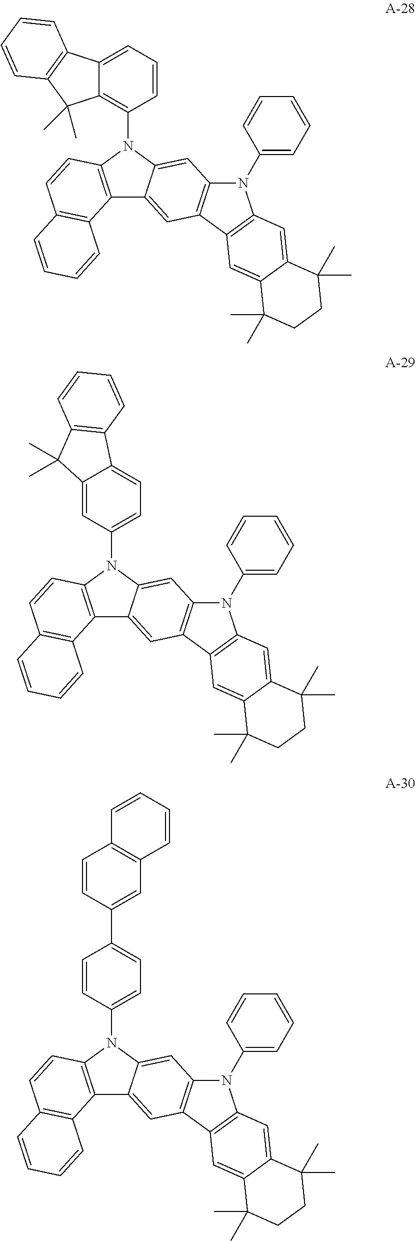 [SMILES: CC1(C)CCC(C)(C)c2cc3c(cc21)c1cc2c4c5ccccc5ccc4n(-c4cccc5c4C(C)(C)c4ccccc4-5)c2cc1n3-c1ccccc1 ; CC1(C)CCC(C)(C)c2cc3c(cc21)c1cc2c4c5ccccc5ccc4n(-c4ccc5c(c4)C(C)(C)c4ccccc4-5)c2cc1n3-c1ccccc1 ; CC1(C)CCC(C)(C)c2cc3c(cc21)c1cc2c4c5ccccc5ccc4n(-c4ccc(-c5ccc6ccccc6c5)cc4)c2cc1n3-c1ccccc1]

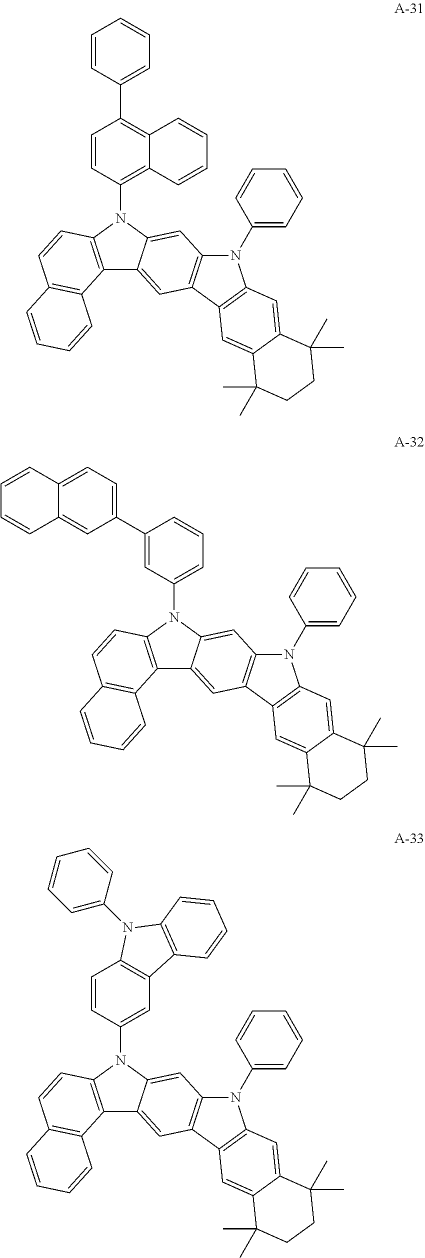 [SMILES: CC1(C)CCC(C)(C)c2cc3c(cc21)c1cc2c4c5ccccc5ccc4n(-c4ccc(-c5ccccc5)c5ccccc45)c2cc1n3-c1ccccc1 ; CC1(C)CCC(C)(C)c2cc3c(cc21)c1cc2c4c5ccccc5ccc4n(-c4cccc(-c5ccc6ccccc6c5)c4)c2cc1n3-c1ccccc1 ; CC1(C)CCC(C)(C)c2cc3c(cc21)c1cc2c4c5ccccc5ccc4n(-c4ccc5c(c4)c4ccccc4n5-c4ccccc4)c2cc1n3-c1ccccc1]

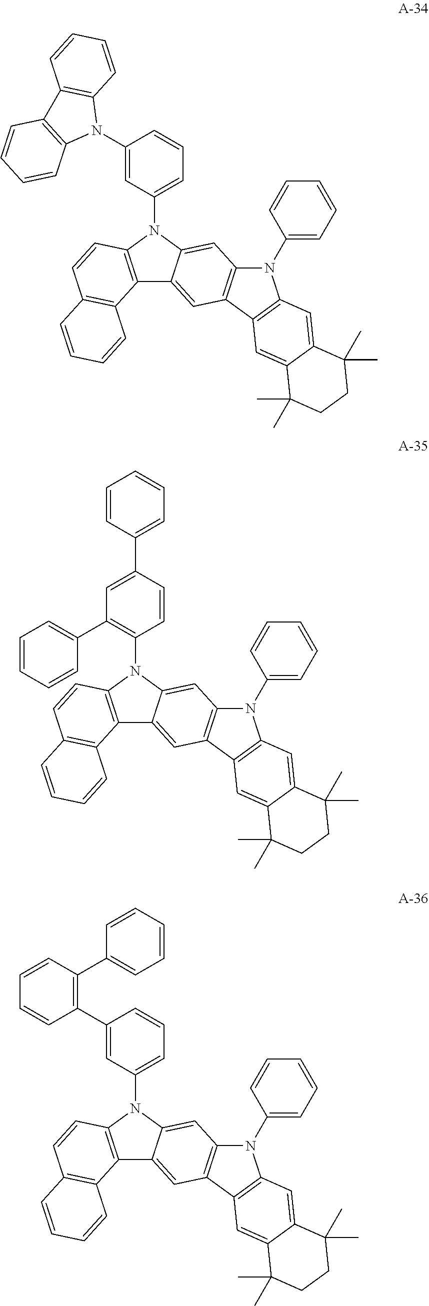 [SMILES: CC1(C)CCC(C)(C)c2cc3c(cc21)c1cc2c4c5ccccc5ccc4n(-c4cccc(-n5c6ccccc6c6ccccc65)c4)c2cc1n3-c1ccccc1 ; CC1(C)CCC(C)(C)c2cc3c(cc21)c1cc2c4c5ccccc5ccc4n(-c4ccc(-c5ccccc5)cc4-c4ccccc4)c2cc1n3-c1ccccc1 ; CC1(C)CCC(C)(C)c2cc3c(cc21)c1cc2c4c5ccccc5ccc4n(-c4cccc(-c5ccccc5-c5ccccc5)c4)c2cc1n3-c1ccccc1]

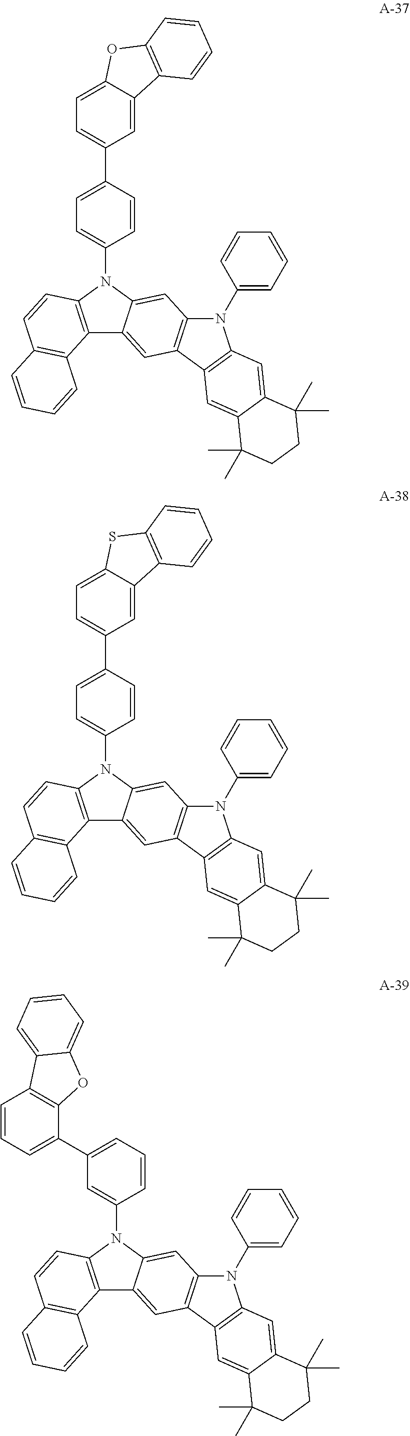

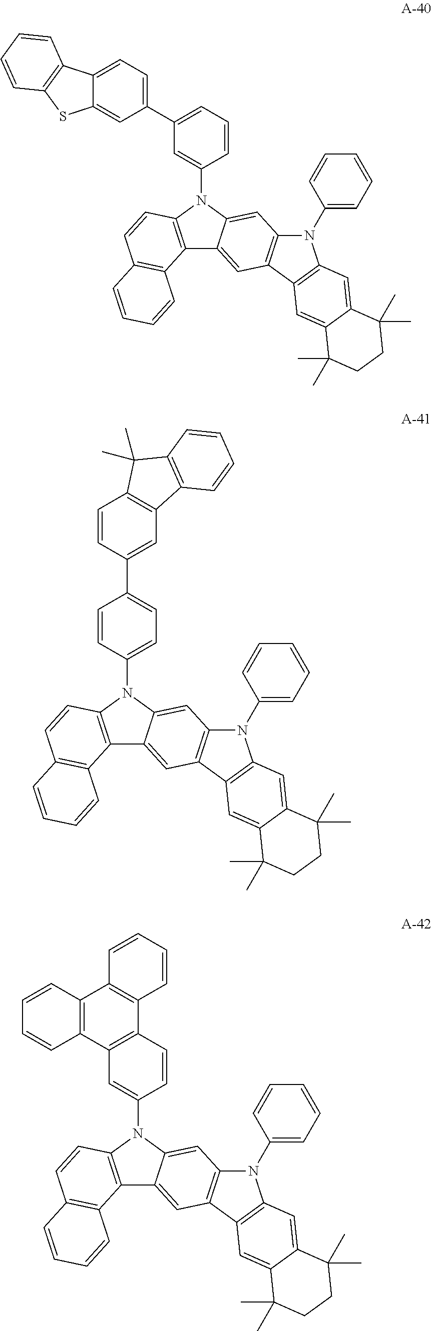 [SMILES: CC1(C)CCC(C)(C)c2cc3c(cc21)c1cc2c4c5ccccc5ccc4n(-c4cccc(-c5ccc6c(c5)sc5ccccc56)c4)c2cc1n3-c1ccccc1 ; CC1(C)CCC(C)(C)c2cc3c(cc21)c1cc2c4c5ccccc5ccc4n(-c4ccc(-c5ccc6c(c5)-c5ccccc5C6(C)C)cc4)c2cc1n3-c1ccccc1 ; CC1(C)CCC(C)(C)c2cc3c(cc21)c1cc2c4c5ccccc5ccc4n(-c4ccc5c6ccccc6c6ccccc6c5c4)c2cc1n3-c1ccccc1]

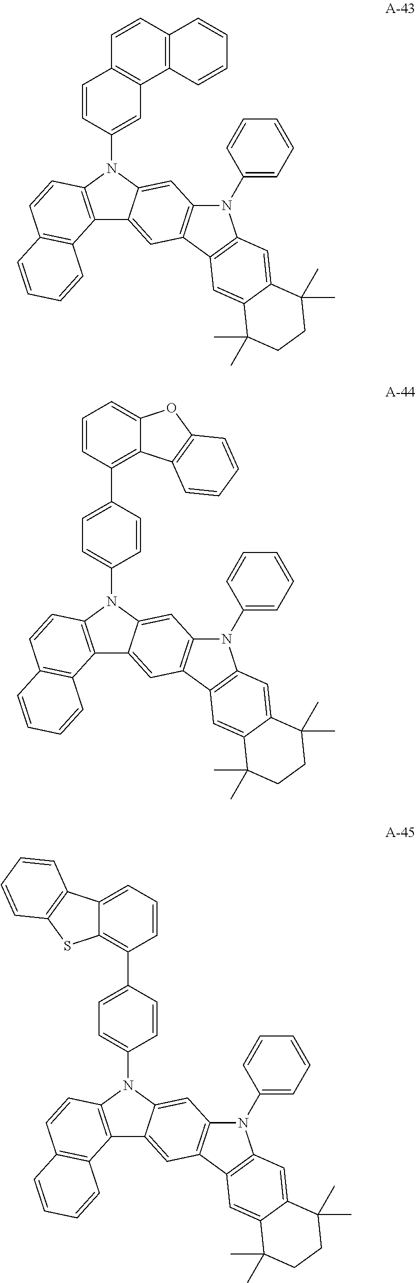 [SMILES: CC1(C)CCC(C)(C)c2cc3c(cc21)c1cc2c4c5ccccc5ccc4n(-c4ccc5ccc6ccccc6c5c4)c2cc1n3-c1ccccc1 ; CC1(C)CCC(C)(C)c2cc3c(cc21)c1cc2c4c5ccccc5ccc4n(-c4ccc(-c5cccc6oc7ccccc7c56)cc4)c2cc1n3-c1ccccc1 ; CC1(C)CCC(C)(C)c2cc3c(cc21)c1cc2c4c5ccccc5ccc4n(-c4ccc(-c5cccc6c5sc5ccccc56)cc4)c2cc1n3-c1ccccc1]

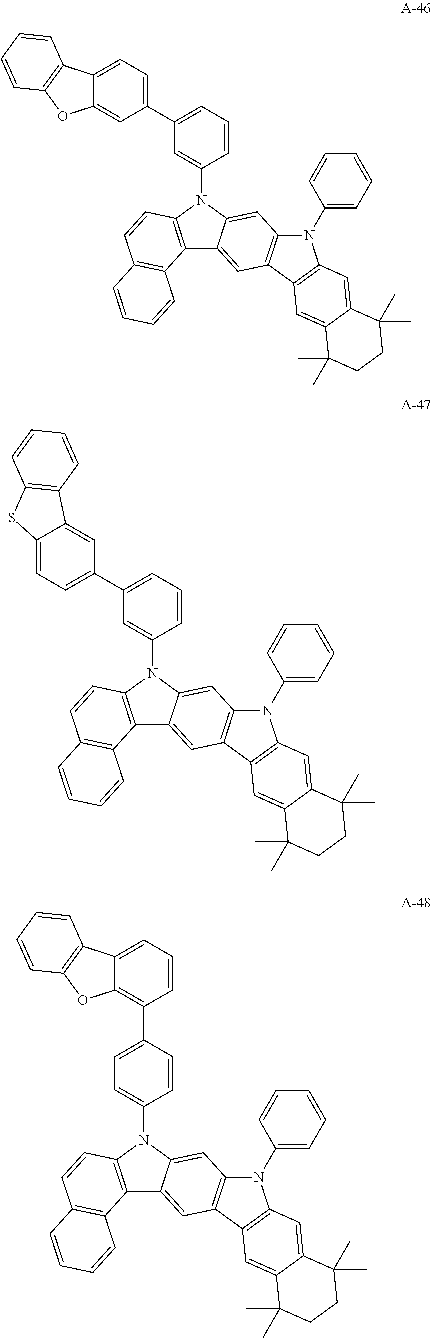 [SMILES: CC1(C)CCC(C)(C)c2cc3c(cc21)c1cc2c4c5ccccc5ccc4n(-c4cccc(-c5ccc6c(c5)oc5ccccc56)c4)c2cc1n3-c1ccccc1 ; CC1(C)CCC(C)(C)c2cc3c(cc21)c1cc2c4c5ccccc5ccc4n(-c4cccc(-c5ccc6sc7ccccc7c6c5)c4)c2cc1n3-c1ccccc1 ; CC1(C)CCC(C)(C)c2cc3c(cc21)c1cc2c4c5ccccc5ccc4n(-c4ccc(-c5cccc6c5oc5ccccc56)cc4)c2cc1n3-c1ccccc1]

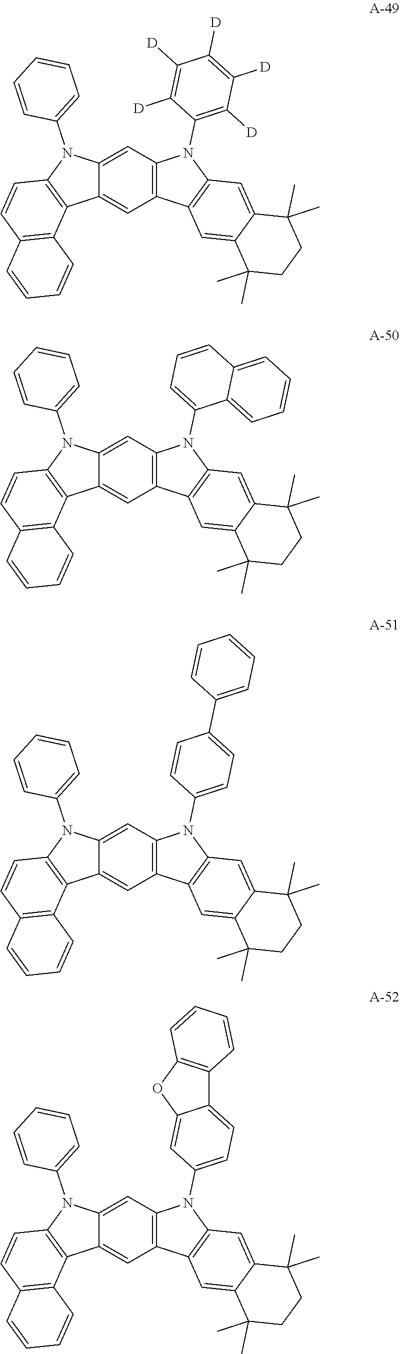

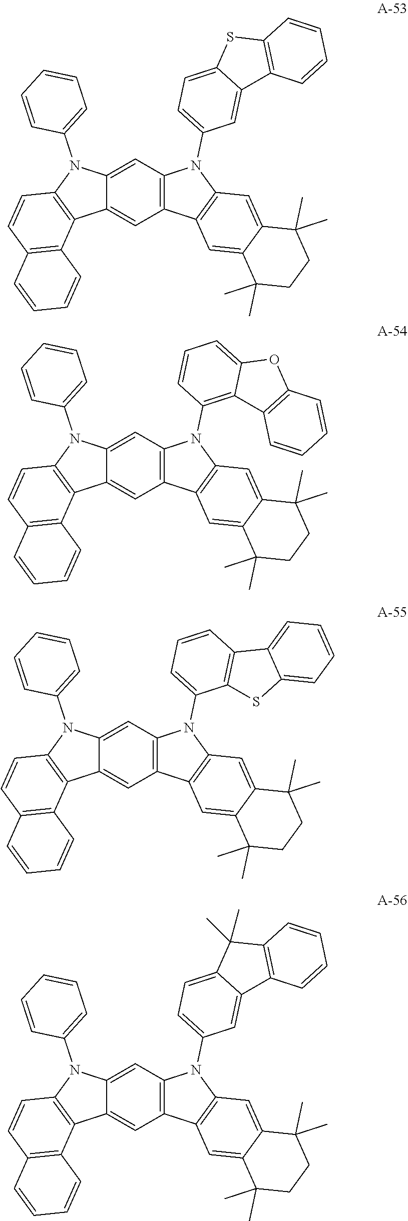

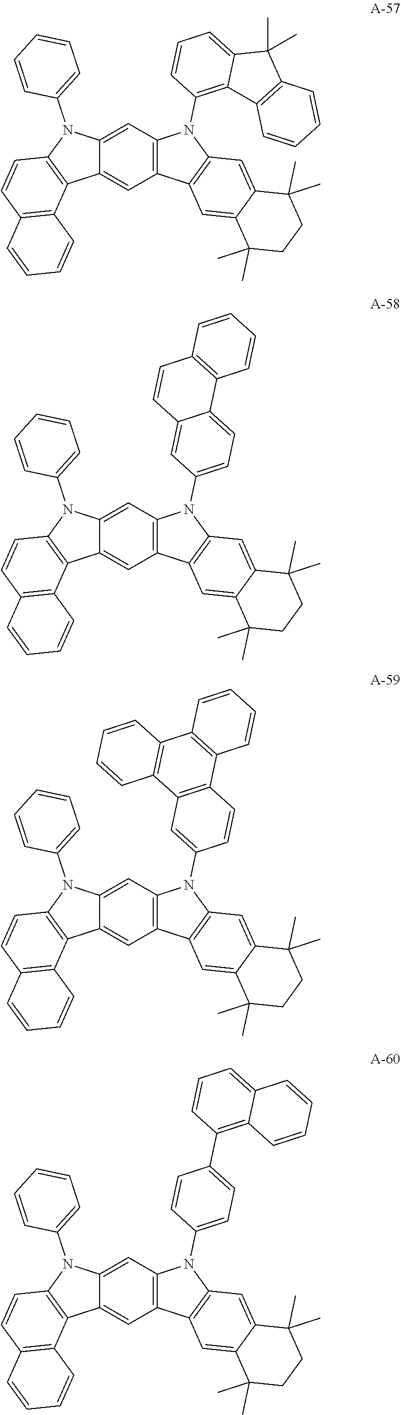

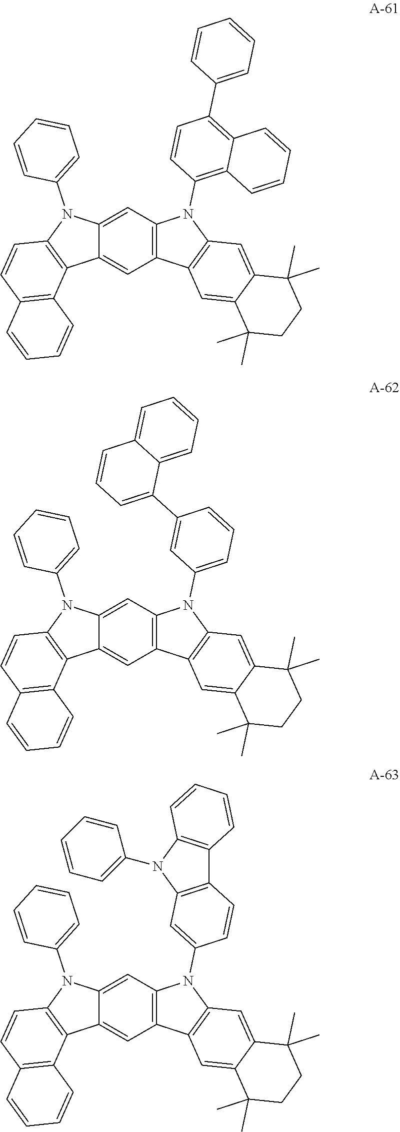

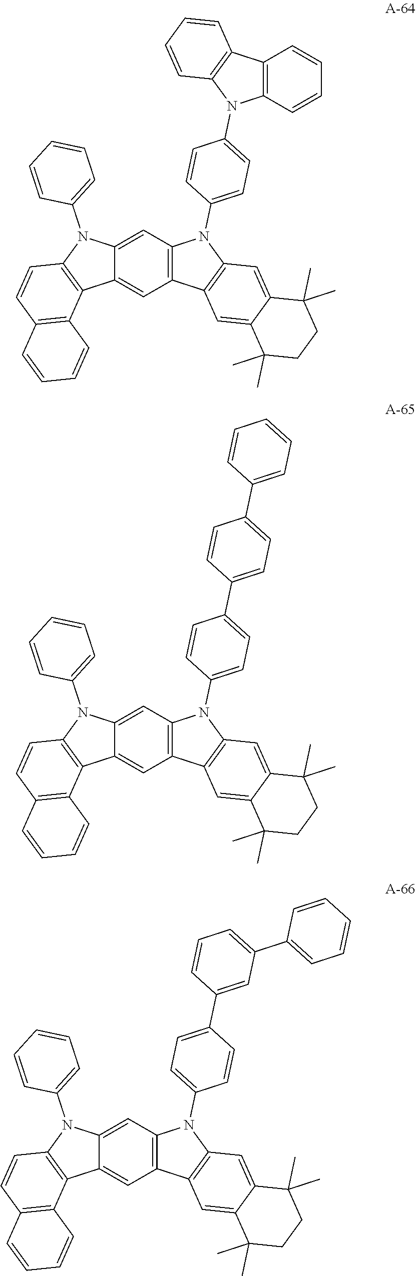

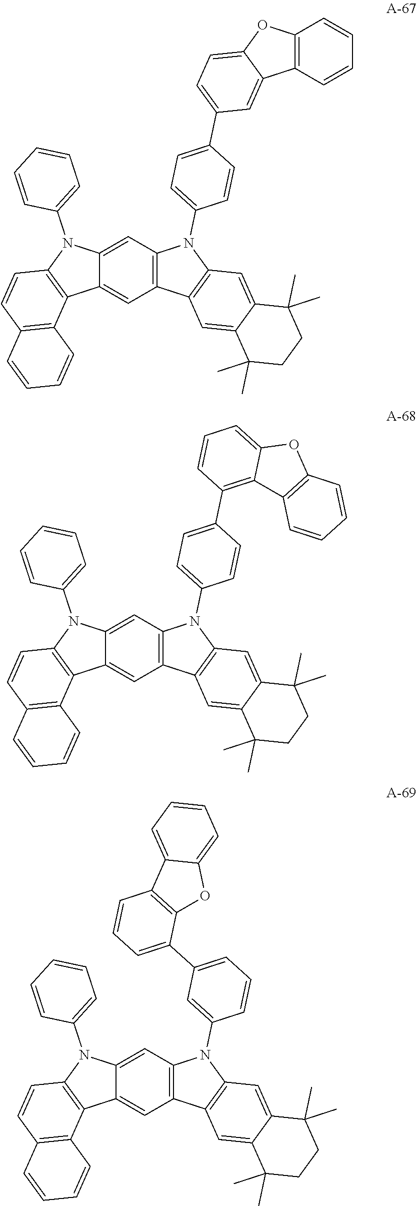

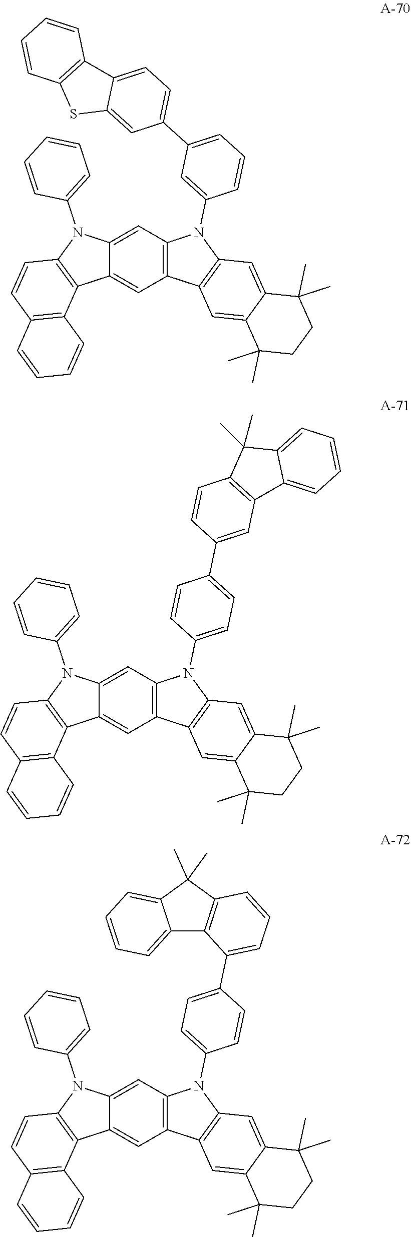

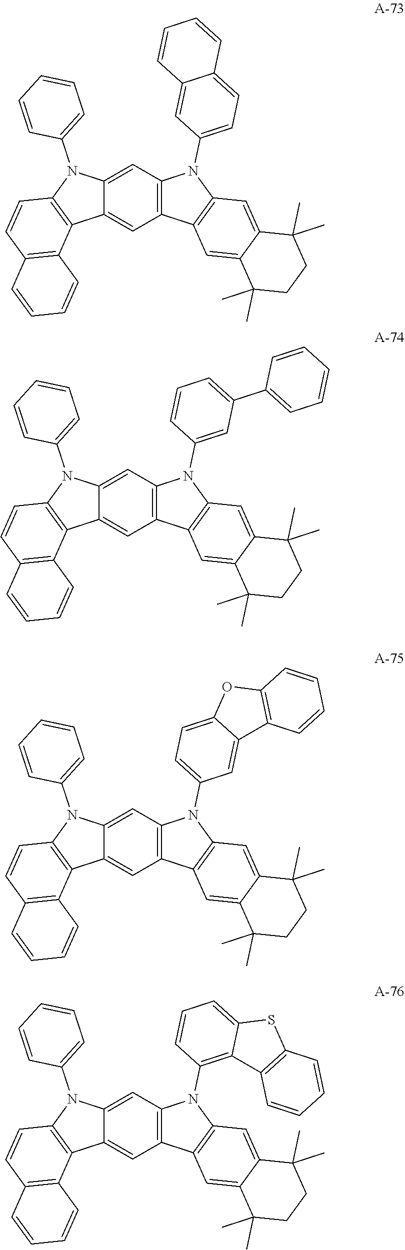

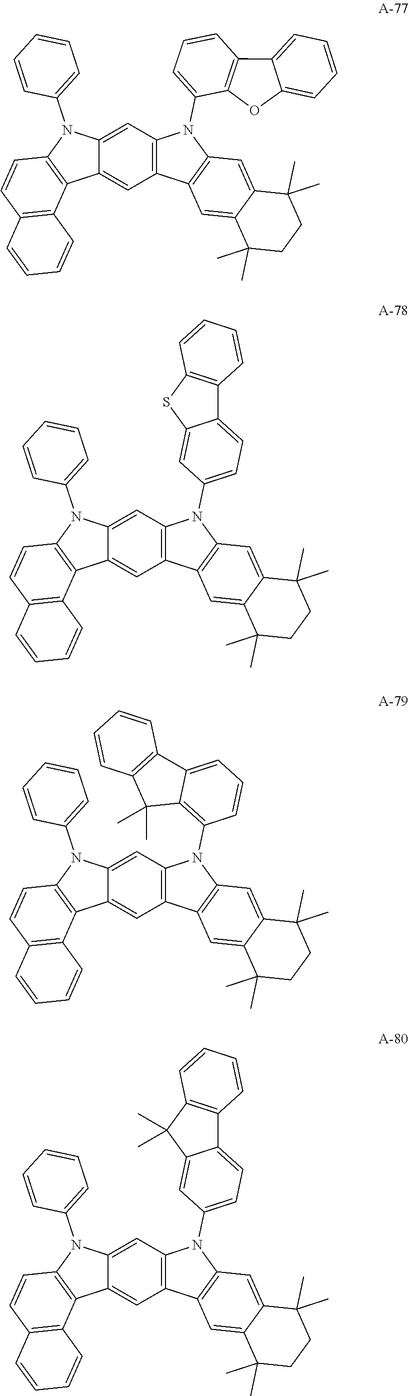 [SMILES: CC1(C)CCC(C)(C)c2cc3c(cc21)c1cc2c4c5ccccc5ccc4n(-c4ccccc4)c2cc1n3-c1cccc2c1oc1ccccc12 ; CC1(C)CCC(C)(C)c2cc3c(cc21)c1cc2c4c5ccccc5ccc4n(-c4ccccc4)c2cc1n3-c1ccc2c(c1)sc1ccccc12 ; CC1(C)CCC(C)(C)c2cc3c(cc21)c1cc2c4c5ccccc5ccc4n(-c4ccccc4)c2cc1n3-c1cccc2c1C(C)(C)c1ccccc1-2 ; CC1(C)CCC(C)(C)c2cc3c(cc21)c1cc2c4c5ccccc5ccc4n(-c4ccccc4)c2cc1n3-c1ccc2c(c1)C(C)(C)c1ccccc1-2]

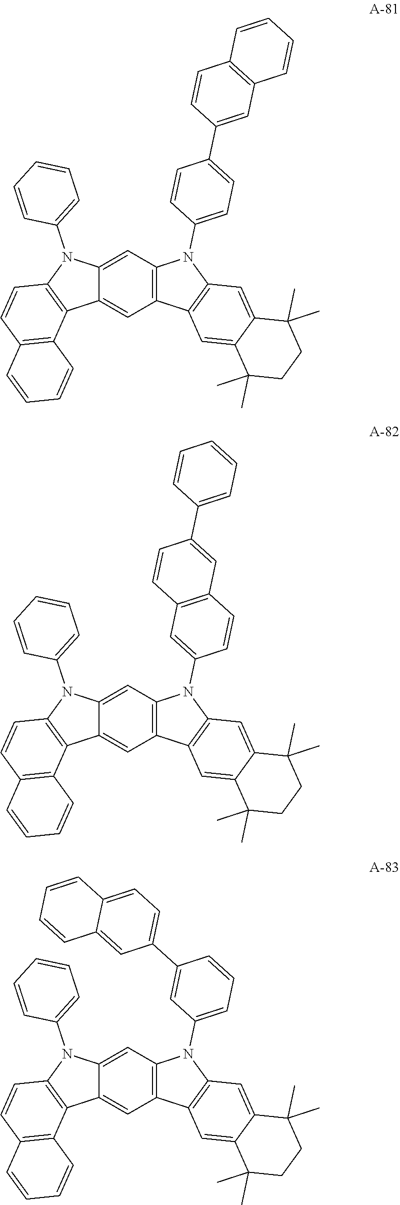

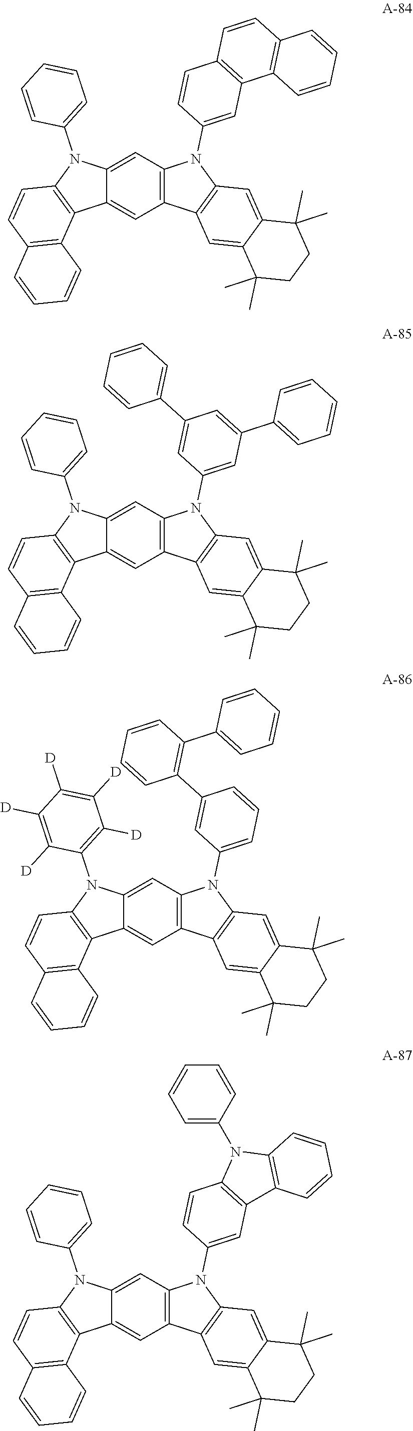

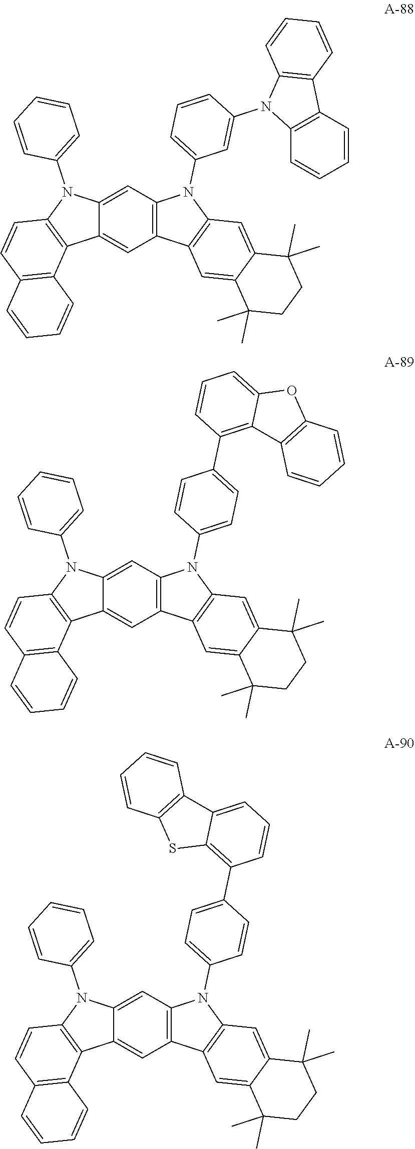

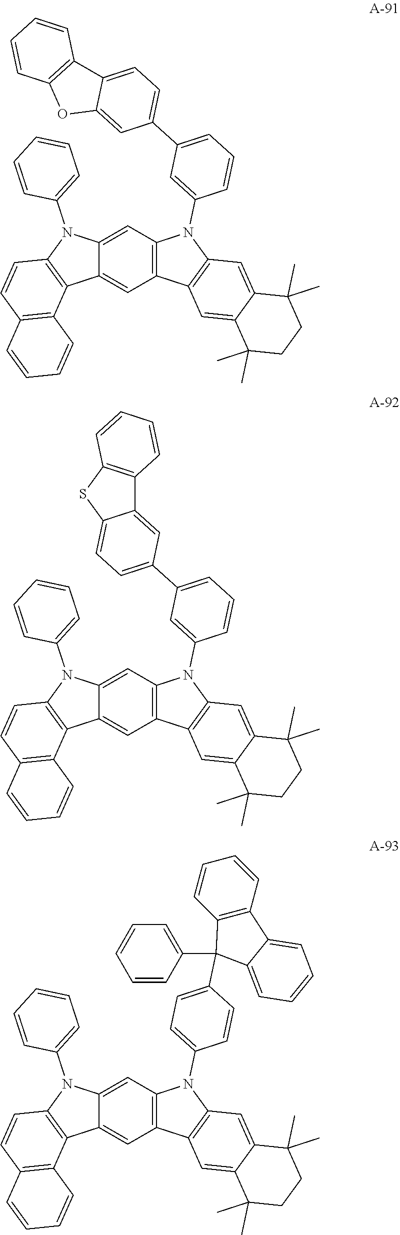

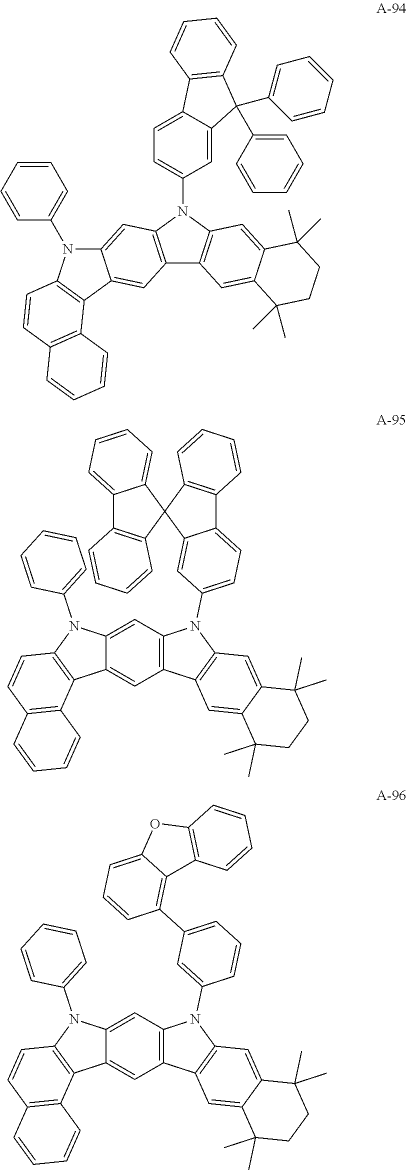

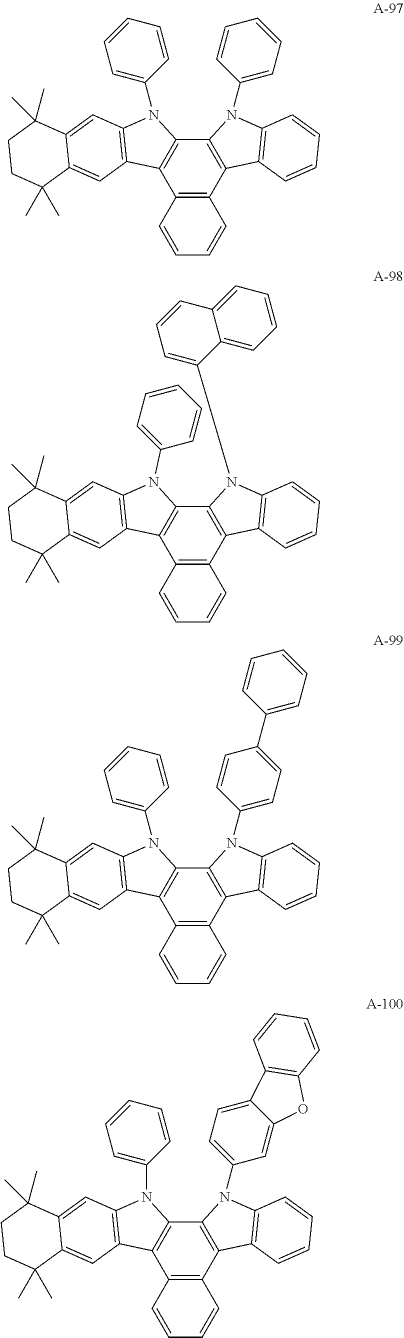

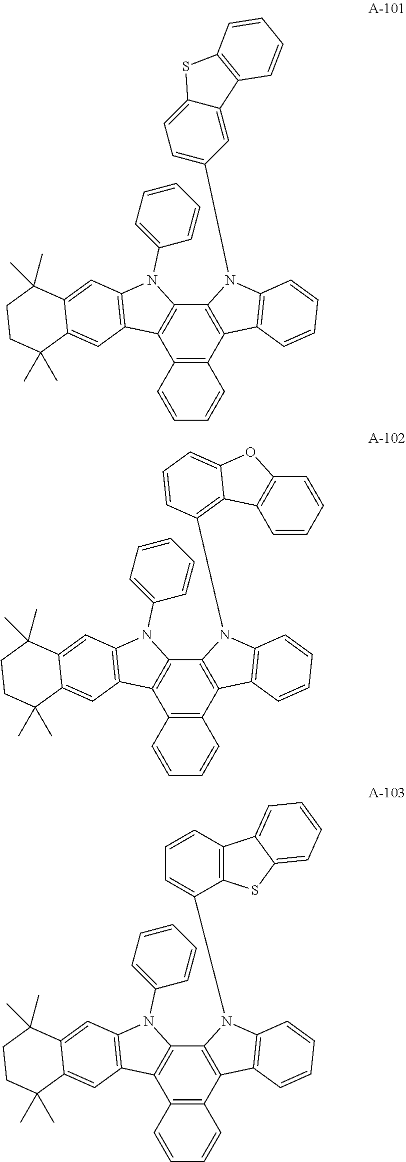

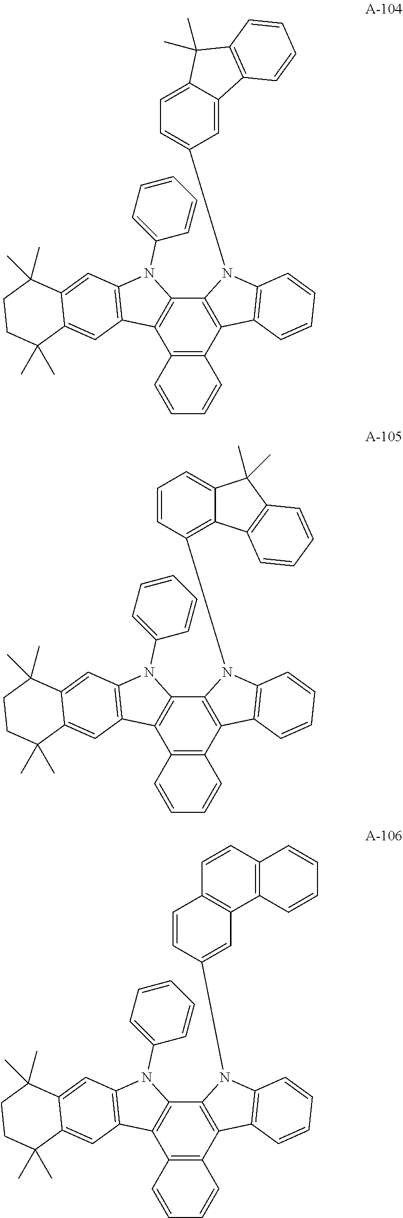

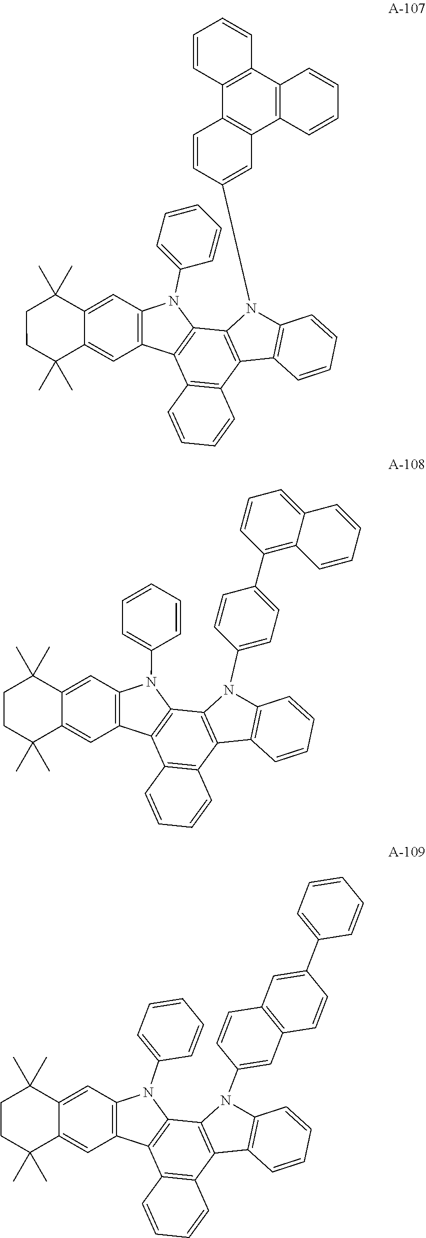 [SMILES: CC1(C)CCC(C)(C)c2cc3c(cc21)c1c2ccccc2c2c4ccccc4n(-c4ccc5c6ccccc6c6ccccc6c5c4)c2c1n3-c1ccccc1 ; CC1(C)CCC(C)(C)c2cc3c(cc21)c1c2ccccc2c2c4ccccc4n(-c4ccc(-c5cccc6ccccc56)cc4)c2c1n3-c1ccccc1 ; CC1(C)CCC(C)(C)c2cc3c(cc21)c1c2ccccc2c2c4ccccc4n(-c4ccc5cc(-c6ccccc6)ccc5c4)c2c1n3-c1ccccc1]

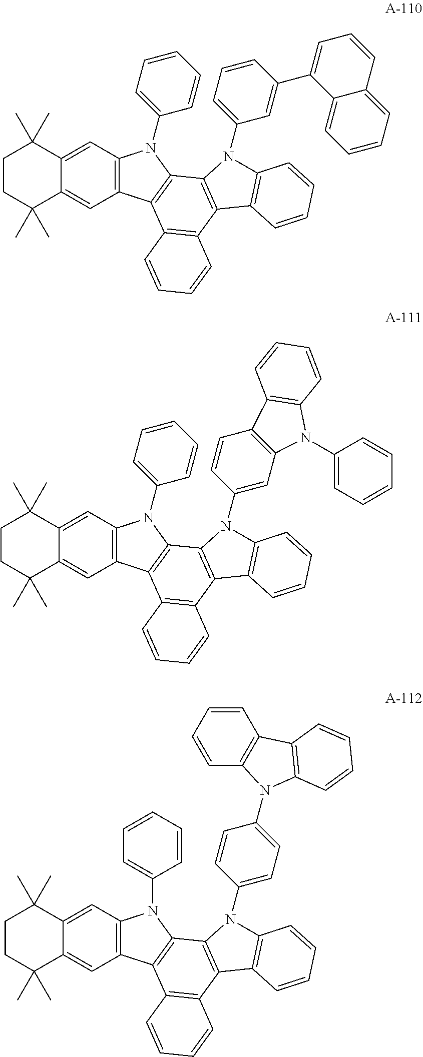 [SMILES: CC1(C)CCC(C)(C)c2cc3c(cc21)c1c2ccccc2c2c4ccccc4n(-c4cccc(-c5cccc6ccccc56)c4)c2c1n3-c1ccccc1 ; CC1(C)CCC(C)(C)c2cc3c(cc21)c1c2ccccc2c2c4ccccc4n(-c4ccc5c6ccccc6n(-c6ccccc6)c5c4)c2c1n3-c1ccccc1 ; CC1(C)CCC(C)(C)c2cc3c(cc21)c1c2ccccc2c2c4ccccc4n(-c4ccc(-n5c6ccccc6c6ccccc65)cc4)c2c1n3-c1ccccc1]

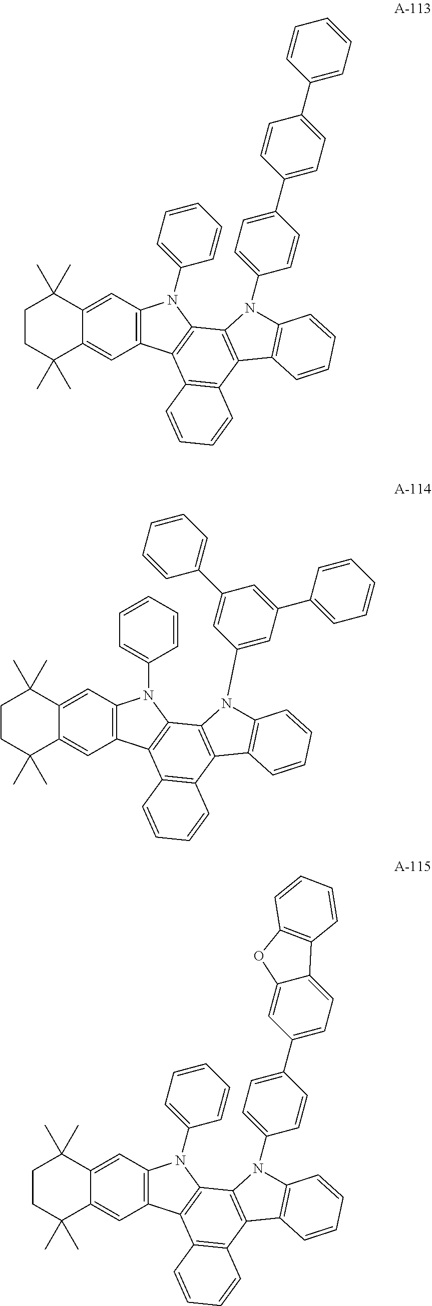 [SMILES: CC1(C)CCC(C)(C)c2cc3c(cc21)c1c2ccccc2c2c4ccccc4n(-c4ccc(-c5ccc(-c6ccccc6)cc5)cc4)c2c1n3-c1ccccc1 ; CC1(C)CCC(C)(C)c2cc3c(cc21)c1c2ccccc2c2c4ccccc4n(-c4cc(-c5ccccc5)cc(-c5ccccc5)c4)c2c1n3-c1ccccc1 ; CC1(C)CCC(C)(C)c2cc3c(cc21)c1c2ccccc2c2c4ccccc4n(-c4ccc(-c5ccc6c(c5)oc5ccccc56)cc4)c2c1n3-c1ccccc1]

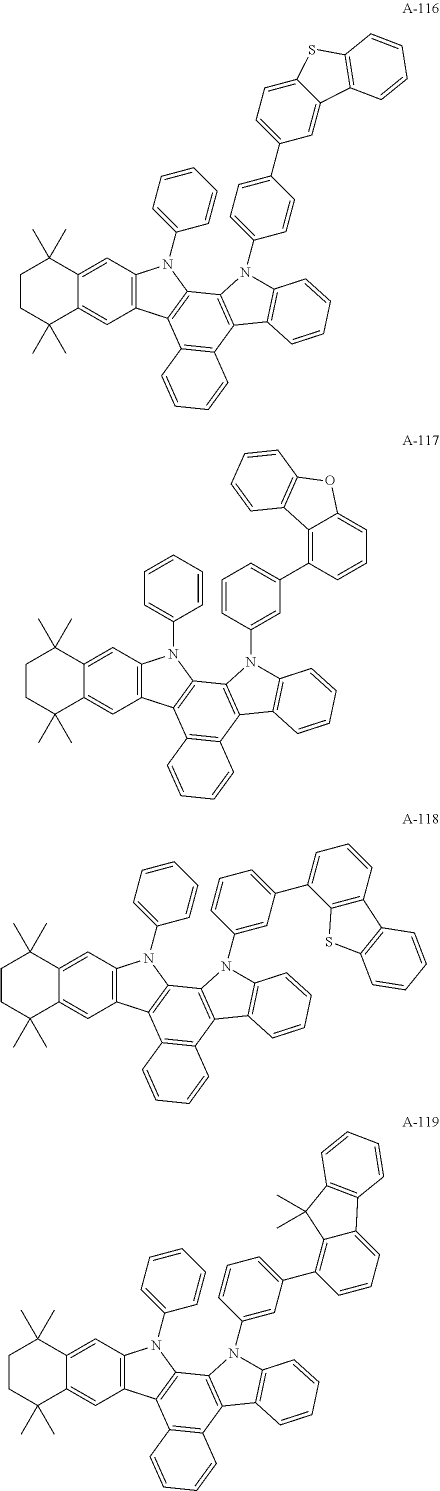 [SMILES: CC1(C)CCC(C)(C)c2cc3c(cc21)c1c2ccccc2c2c4ccccc4n(-c4ccc(-c5ccc6sc7ccccc7c6c5)cc4)c2c1n3-c1ccccc1 ; CC1(C)CCC(C)(C)c2cc3c(cc21)c1c2ccccc2c2c4ccccc4n(-c4cccc(-c5cccc6oc7ccccc7c56)c4)c2c1n3-c1ccccc1 ; CC1(C)CCC(C)(C)c2cc3c(cc21)c1c2ccccc2c2c4ccccc4n(-c4cccc(-c5cccc6c5sc5ccccc56)c4)c2c1n3-c1ccccc1 ; CC1(C)CCC(C)(C)c2cc3c(cc21)c1c2ccccc2c2c4ccccc4n(-c4cccc(-c5cccc6c5C(C)(C)c5ccccc5-6)c4)c2c1n3-c1ccccc1]

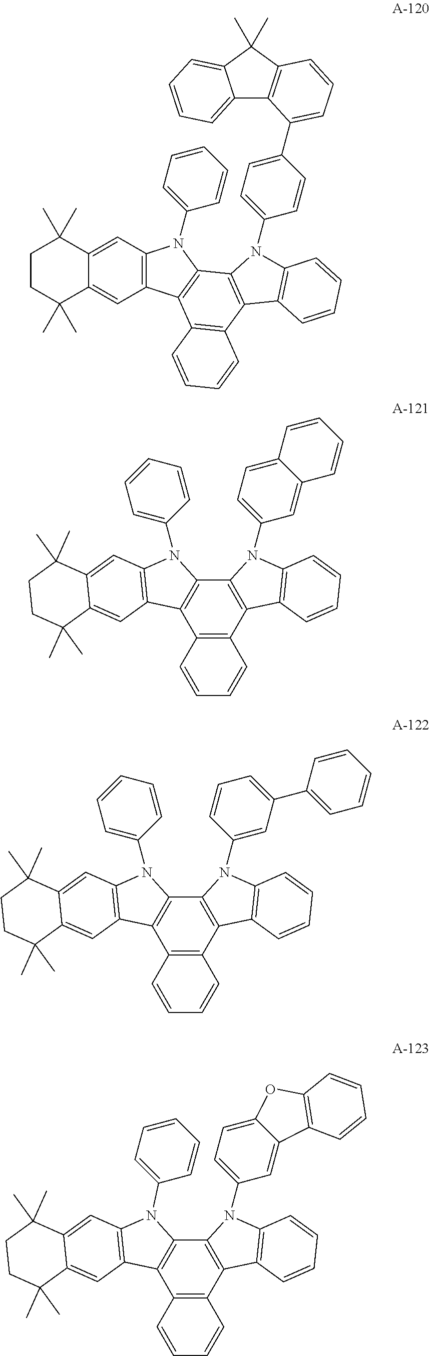 [SMILES: CC1(C)CCC(C)(C)c2cc3c(cc21)c1c2ccccc2c2c4ccccc4n(-c4ccc(-c5cccc6c5-c5ccccc5C6(C)C)cc4)c2c1n3-c1ccccc1 ; CC1(C)CCC(C)(C)c2cc3c(cc21)c1c2ccccc2c2c4ccccc4n(-c4ccc5ccccc5c4)c2c1n3-c1ccccc1 ; CC1(C)CCC(C)(C)c2cc3c(cc21)c1c2ccccc2c2c4ccccc4n(-c4cccc(-c5ccccc5)c4)c2c1n3-c1ccccc1 ; CC1(C)CCC(C)(C)c2cc3c(cc21)c1c2ccccc2c2c4ccccc4n(-c4ccc5oc6ccccc6c5c4)c2c1n3-c1ccccc1]

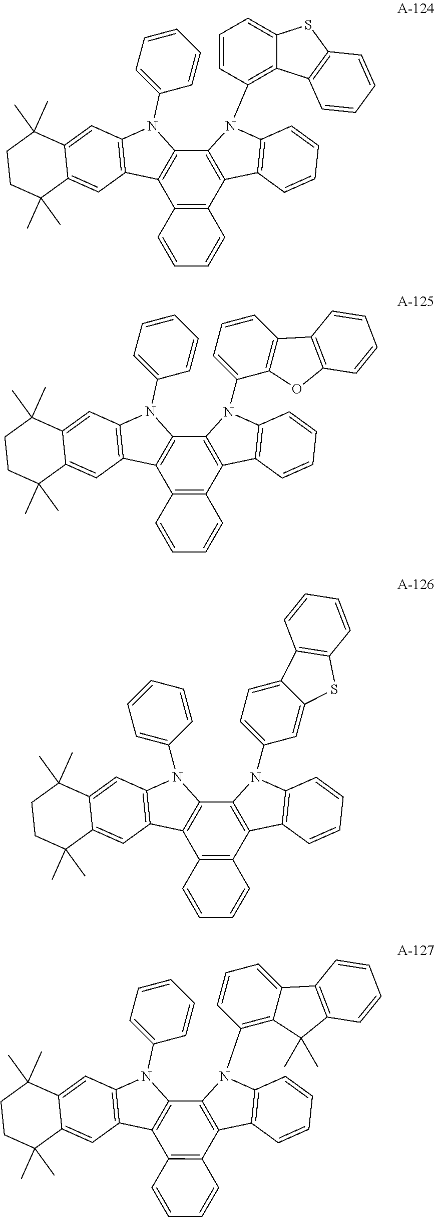 [SMILES: CC1(C)CCC(C)(C)c2cc3c(cc21)c1c2ccccc2c2c4ccccc4n(-c4cccc5sc6ccccc6c45)c2c1n3-c1ccccc1 ; CC1(C)CCC(C)(C)c2cc3c(cc21)c1c2ccccc2c2c4ccccc4n(-c4cccc5c4oc4ccccc45)c2c1n3-c1ccccc1 ; CC1(C)CCC(C)(C)c2cc3c(cc21)c1c2ccccc2c2c4ccccc4n(-c4ccc5c(c4)sc4ccccc45)c2c1n3-c1ccccc1 ; CC1(C)CCC(C)(C)c2cc3c(cc21)c1c2ccccc2c2c4ccccc4n(-c4cccc5c4C(C)(C)c4ccccc4-5)c2c1n3-c1ccccc1]

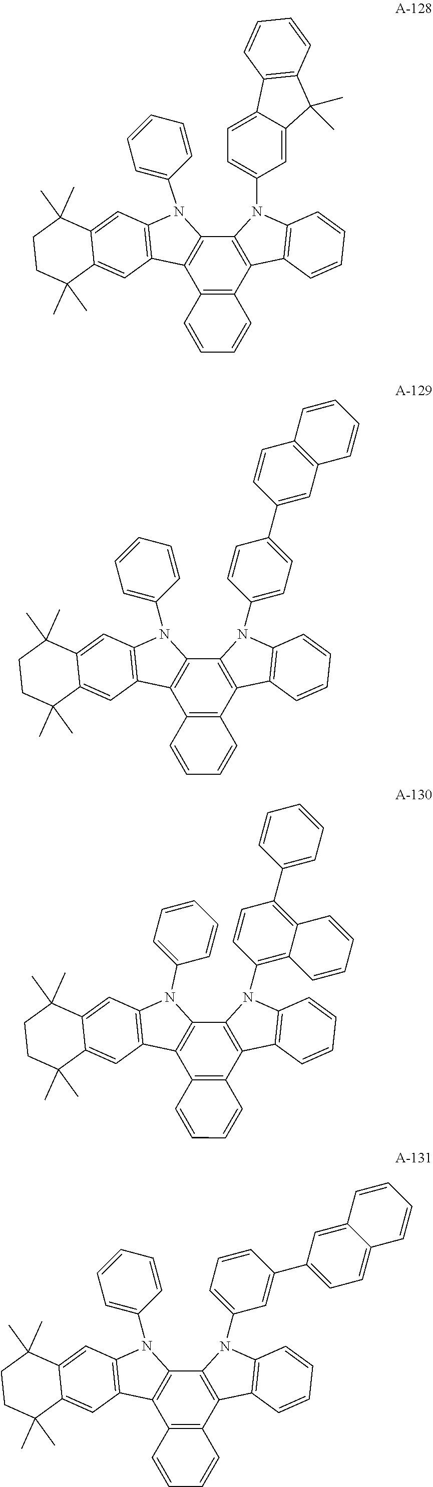 [SMILES: CC1(C)CCC(C)(C)c2cc3c(cc21)c1c2ccccc2c2c4ccccc4n(-c4ccc5c(c4)C(C)(C)c4ccccc4-5)c2c1n3-c1ccccc1 ; CC1(C)CCC(C)(C)c2cc3c(cc21)c1c2ccccc2c2c4ccccc4n(-c4ccc(-c5ccc6ccccc6c5)cc4)c2c1n3-c1ccccc1 ; CC1(C)CCC(C)(C)c2cc3c(cc21)c1c2ccccc2c2c4ccccc4n(-c4ccc(-c5ccccc5)c5ccccc45)c2c1n3-c1ccccc1 ; CC1(C)CCC(C)(C)c2cc3c(cc21)c1c2ccccc2c2c4ccccc4n(-c4cccc(-c5ccc6ccccc6c5)c4)c2c1n3-c1ccccc1]

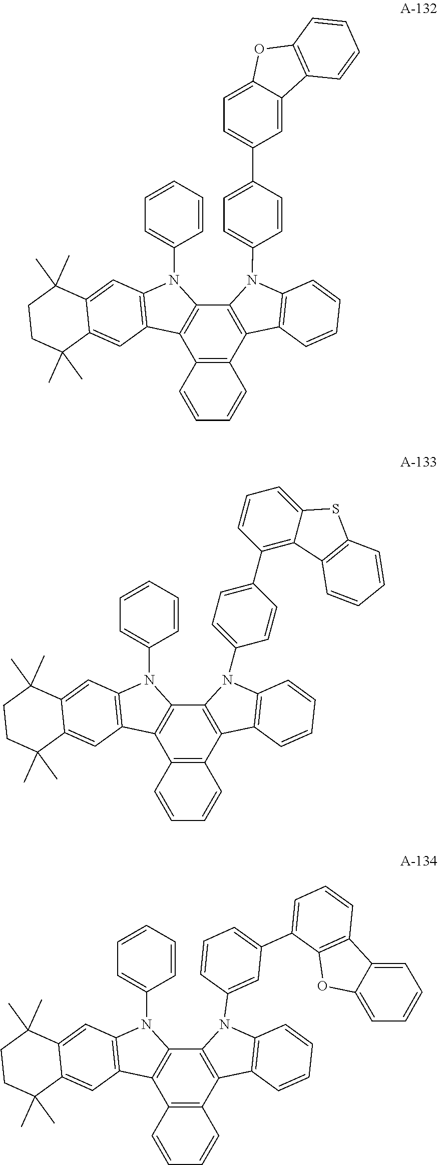 [SMILES: CC1(C)CCC(C)(C)c2cc3c(cc21)c1c2ccccc2c2c4ccccc4n(-c4ccc(-c5ccc6oc7ccccc7c6c5)cc4)c2c1n3-c1ccccc1 ; CC1(C)CCC(C)(C)c2cc3c(cc21)c1c2ccccc2c2c4ccccc4n(-c4ccc(-c5cccc6sc7ccccc7c56)cc4)c2c1n3-c1ccccc1 ; CC1(C)CCC(C)(C)c2cc3c(cc21)c1c2ccccc2c2c4ccccc4n(-c4cccc(-c5cccc6c5oc5ccccc56)c4)c2c1n3-c1ccccc1]

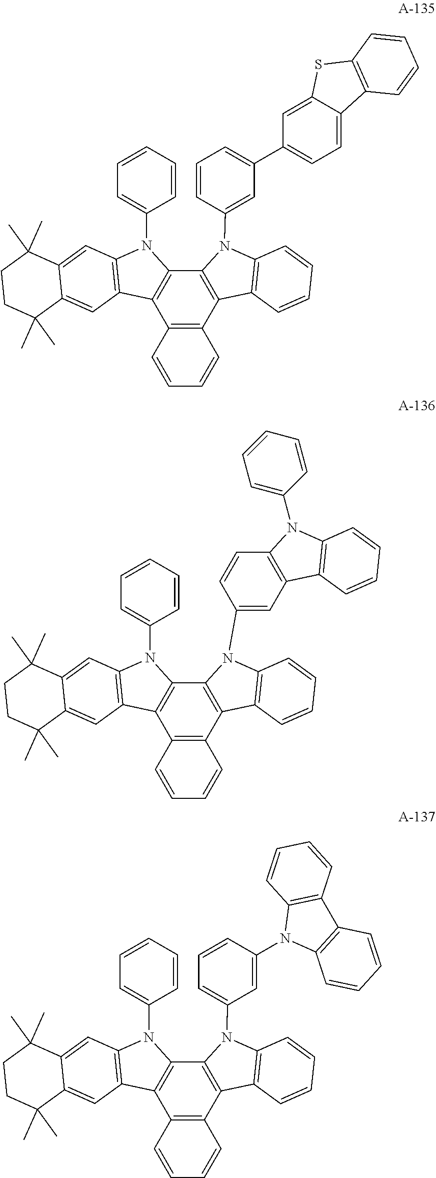 [SMILES: CC1(C)CCC(C)(C)c2cc3c(cc21)c1c2ccccc2c2c4ccccc4n(-c4cccc(-c5ccc6c(c5)sc5ccccc56)c4)c2c1n3-c1ccccc1 ; CC1(C)CCC(C)(C)c2cc3c(cc21)c1c2ccccc2c2c4ccccc4n(-c4ccc5c(c4)c4ccccc4n5-c4ccccc4)c2c1n3-c1ccccc1 ; CC1(C)CCC(C)(C)c2cc3c(cc21)c1c2ccccc2c2c4ccccc4n(-c4cccc(-n5c6ccccc6c6ccccc65)c4)c2c1n3-c1ccccc1]

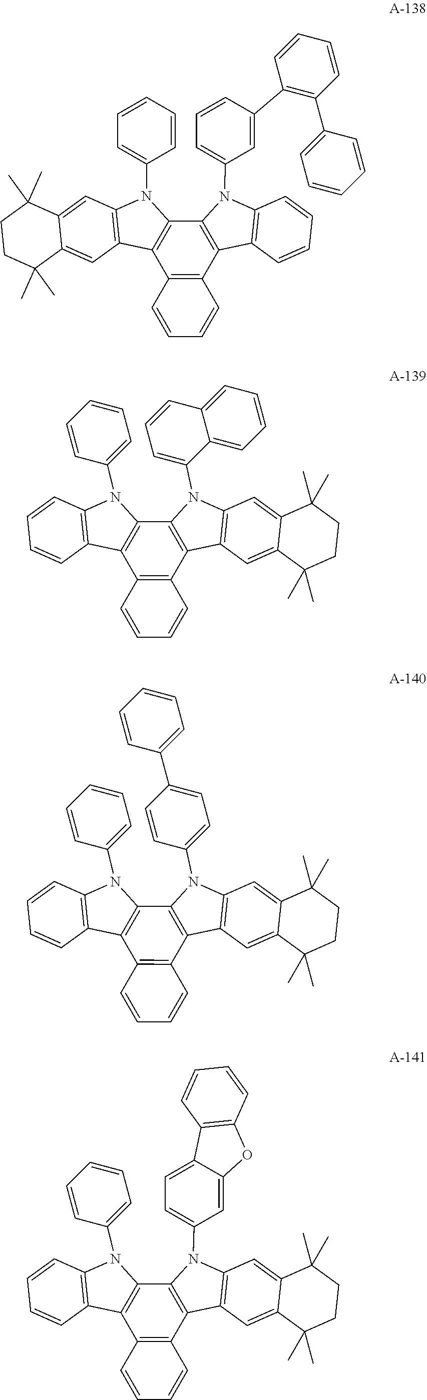 [SMILES: CC1(C)CCC(C)(C)c2cc3c(cc21)c1c2ccccc2c2c4ccccc4n(-c4cccc(-c5ccccc5-c5ccccc5)c4)c2c1n3-c1ccccc1 ; CC1(C)CCC(C)(C)c2cc3c(cc21)c1c2ccccc2c2c4ccccc4n(-c4ccccc4)c2c1n3-c1cccc2ccccc12 ; CC1(C)CCC(C)(C)c2cc3c(cc21)c1c2ccccc2c2c4ccccc4n(-c4ccccc4)c2c1n3-c1ccc(-c2ccccc2)cc1 ; CC1(C)CCC(C)(C)c2cc3c(cc21)c1c2ccccc2c2c4ccccc4n(-c4ccccc4)c2c1n3-c1ccc2c(c1)oc1ccccc12]

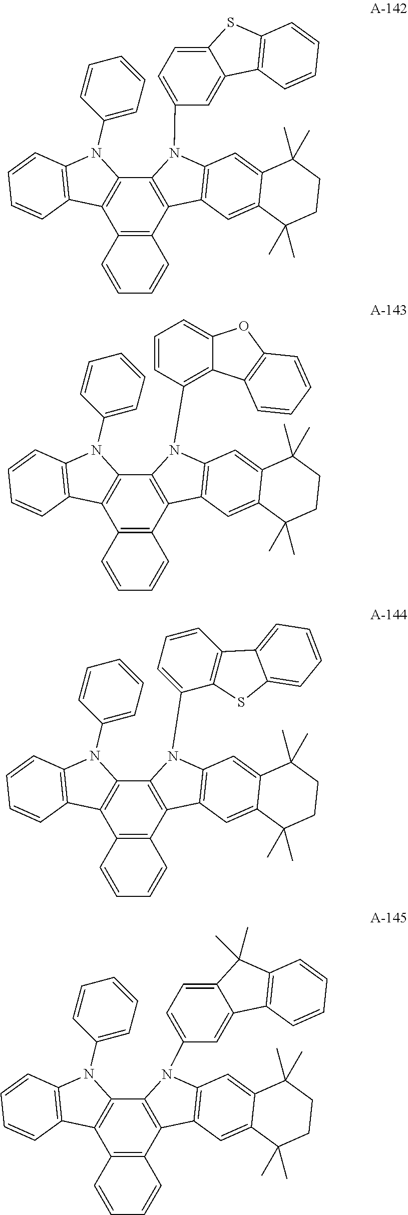

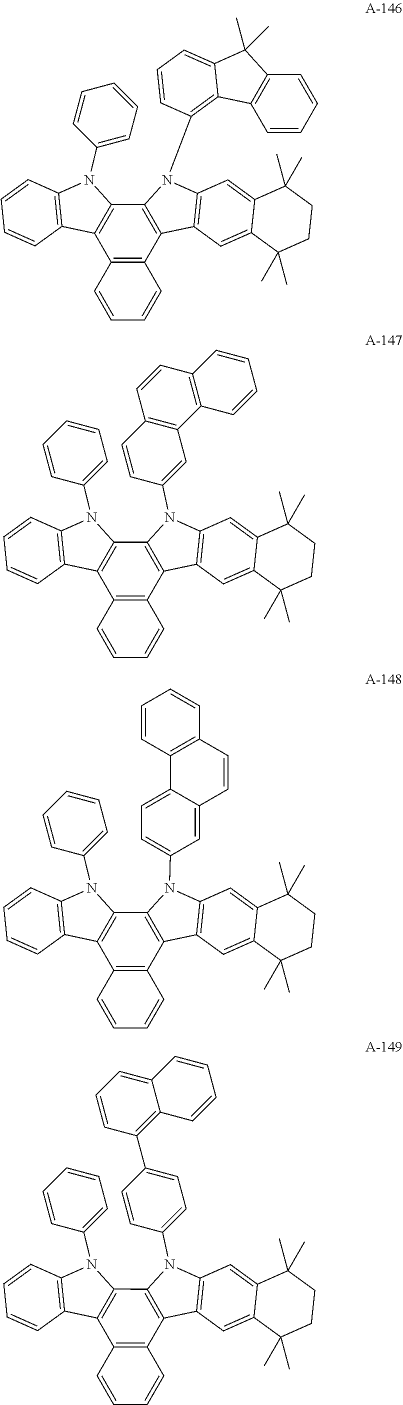

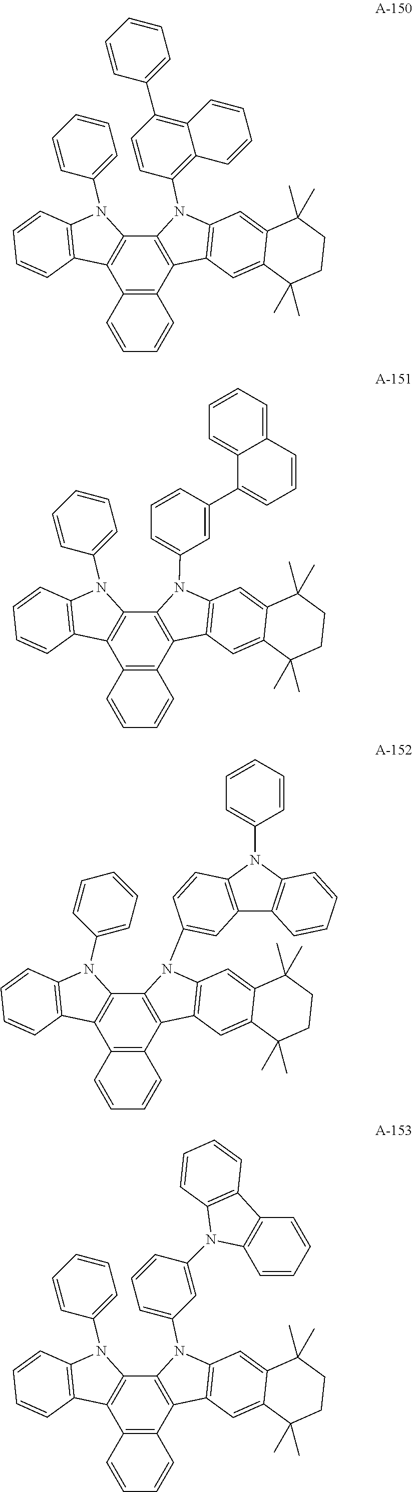 [SMILES: CC1(C)CCC(C)(C)c2cc3c(cc21)c1c2ccccc2c2c4ccccc4n(-c4ccccc4)c2c1n3-c1ccc(-c2ccccc2)c2ccccc12 ; CC1(C)CCC(C)(C)c2cc3c(cc21)c1c2ccccc2c2c4ccccc4n(-c4ccccc4)c2c1n3-c1cccc(-c2cccc3ccccc23)c1 ; CC1(C)CCC(C)(C)c2cc3c(cc21)c1c2ccccc2c2c4ccccc4n(-c4ccccc4)c2c1n3-c1ccc2c(c1)c1ccccc1n2-c1ccccc1 ; CC1(C)CCC(C)(C)c2cc3c(cc21)c1c2ccccc2c2c4ccccc4n(-c4ccccc4)c2c1n3-c1cccc(-n2c3ccccc3c3ccccc32)c1]

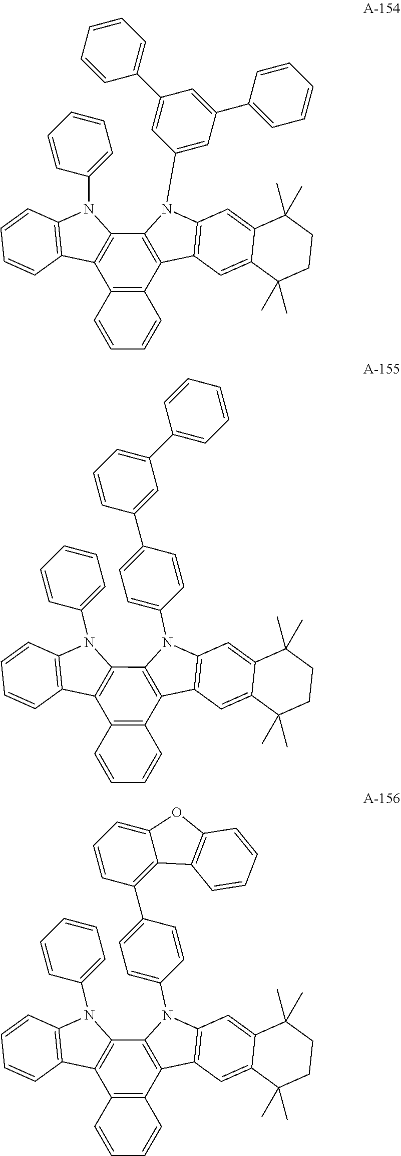

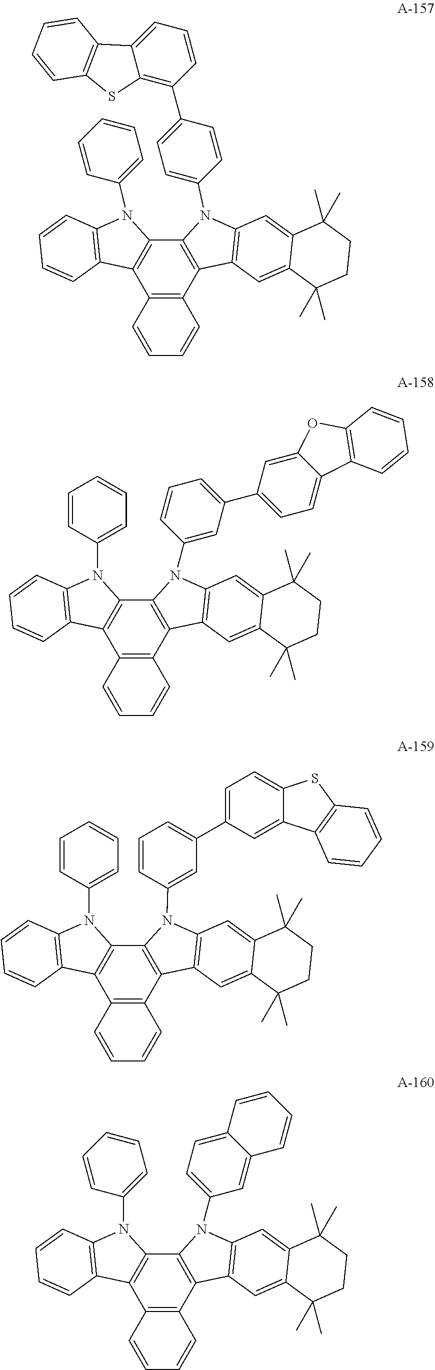 [SMILES: CC1(C)CCC(C)(C)c2cc3c(cc21)c1c2ccccc2c2c4ccccc4n(-c4ccccc4)c2c1n3-c1ccc(-c2cccc3c2sc2ccccc23)cc1 ; CC1(C)CCC(C)(C)c2cc3c(cc21)c1c2ccccc2c2c4ccccc4n(-c4ccccc4)c2c1n3-c1cccc(-c2ccc3c(c2)oc2ccccc23)c1 ; CC1(C)CCC(C)(C)c2cc3c(cc21)c1c2ccccc2c2c4ccccc4n(-c4ccccc4)c2c1n3-c1cccc(-c2ccc3sc4ccccc4c3c2)c1 ; CC1(C)CCC(C)(C)c2cc3c(cc21)c1c2ccccc2c2c4ccccc4n(-c4ccccc4)c2c1n3-c1ccc2ccccc2c1]

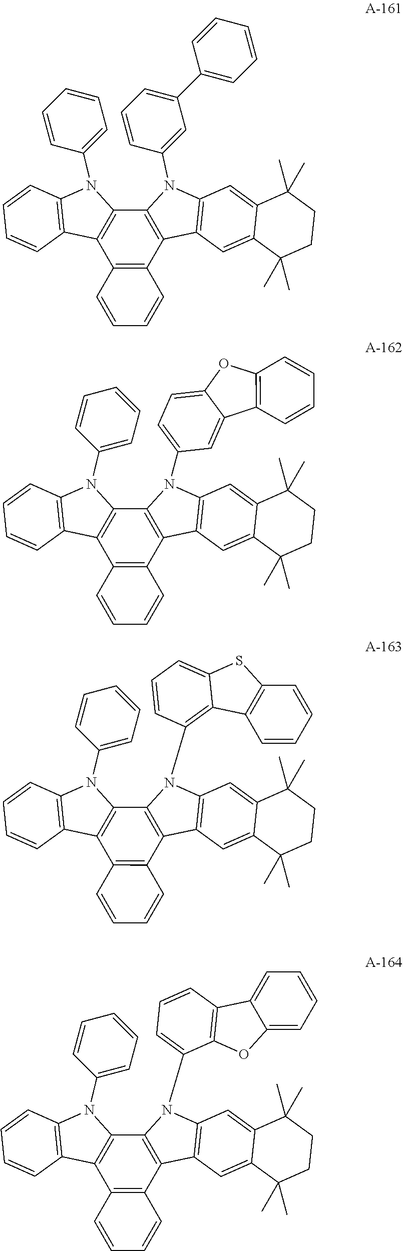 [SMILES: CC1(C)CCC(C)(C)c2cc3c(cc21)c1c2ccccc2c2c4ccccc4n(-c4ccccc4)c2c1n3-c1cccc(-c2ccccc2)c1 ; CC1(C)CCC(C)(C)c2cc3c(cc21)c1c2ccccc2c2c4ccccc4n(-c4ccccc4)c2c1n3-c1ccc2oc3ccccc3c2c1 ; CC1(C)CCC(C)(C)c2cc3c(cc21)c1c2ccccc2c2c4ccccc4n(-c4ccccc4)c2c1n3-c1cccc2sc3ccccc3c12 ; CC1(C)CCC(C)(C)c2cc3c(cc21)c1c2ccccc2c2c4ccccc4n(-c4ccccc4)c2c1n3-c1cccc2c1oc1ccccc12]

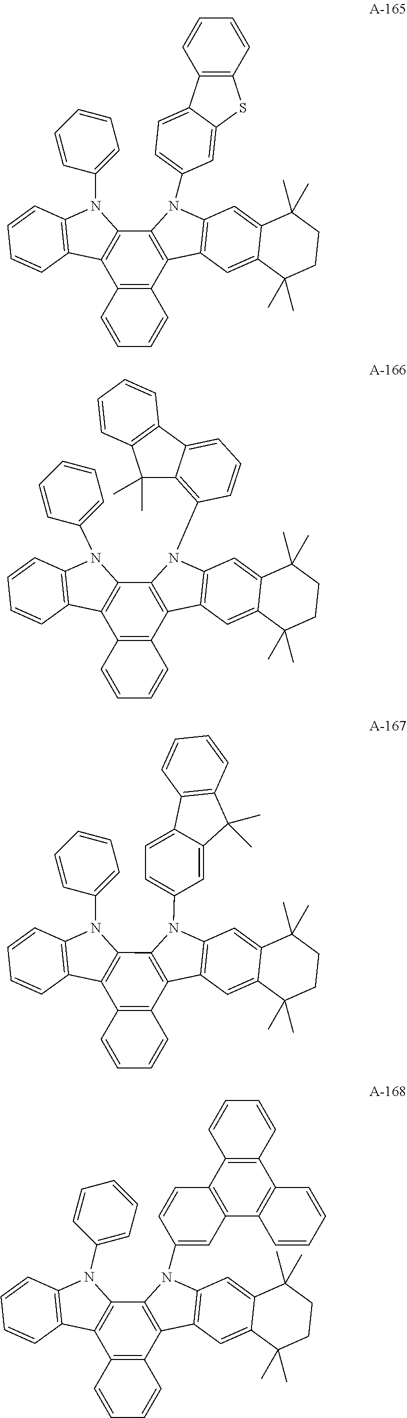

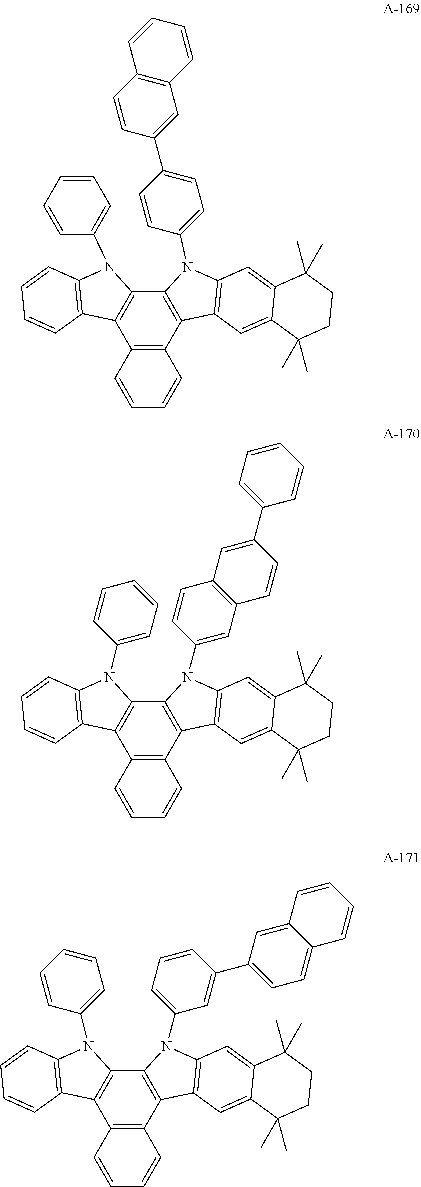 [SMILES: CC1(C)CCC(C)(C)c2cc3c(cc21)c1c2ccccc2c2c4ccccc4n(-c4ccccc4)c2c1n3-c1ccc(-c2ccc3ccccc3c2)cc1 ; CC1(C)CCC(C)(C)c2cc3c(cc21)c1c2ccccc2c2c4ccccc4n(-c4ccccc4)c2c1n3-c1ccc2cc(-c3ccccc3)ccc2c1 ; CC1(C)CCC(C)(C)c2cc3c(cc21)c1c2ccccc2c2c4ccccc4n(-c4ccccc4)c2c1n3-c1cccc(-c2ccc3ccccc3c2)c1]

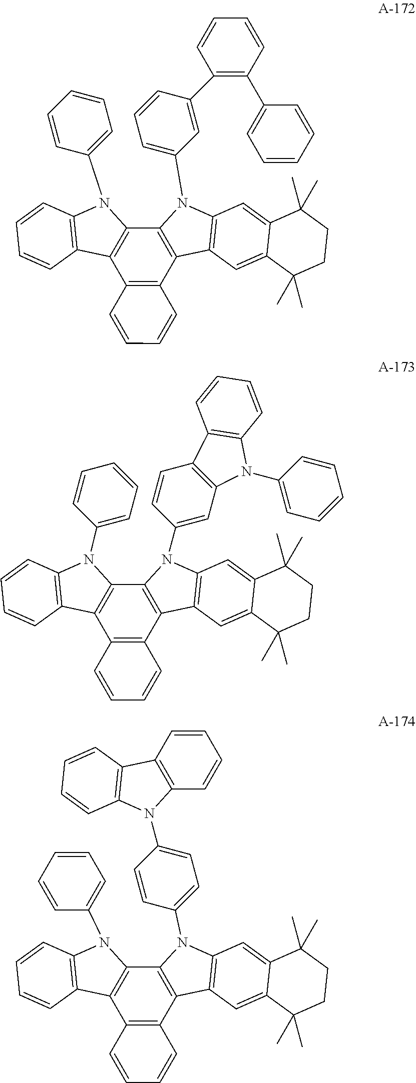

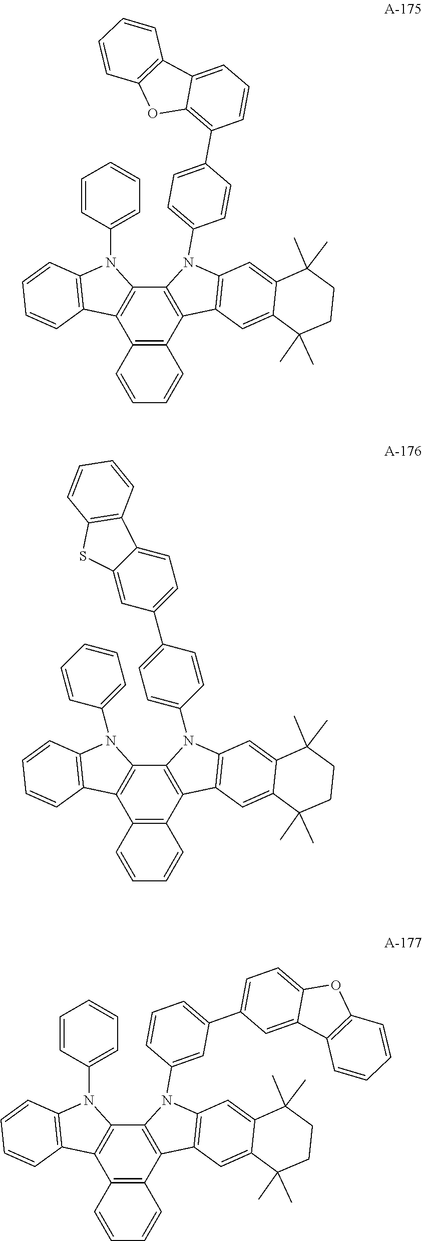 [SMILES: CC1(C)CCC(C)(C)c2cc3c(cc21)c1c2ccccc2c2c4ccccc4n(-c4ccccc4)c2c1n3-c1ccc(-c2cccc3c2oc2ccccc23)cc1 ; CC1(C)CCC(C)(C)c2cc3c(cc21)c1c2ccccc2c2c4ccccc4n(-c4ccccc4)c2c1n3-c1ccc(-c2ccc3c(c2)sc2ccccc23)cc1 ; CC1(C)CCC(C)(C)c2cc3c(cc21)c1c2ccccc2c2c4ccccc4n(-c4ccccc4)c2c1n3-c1cccc(-c2ccc3oc4ccccc4c3c2)c1]

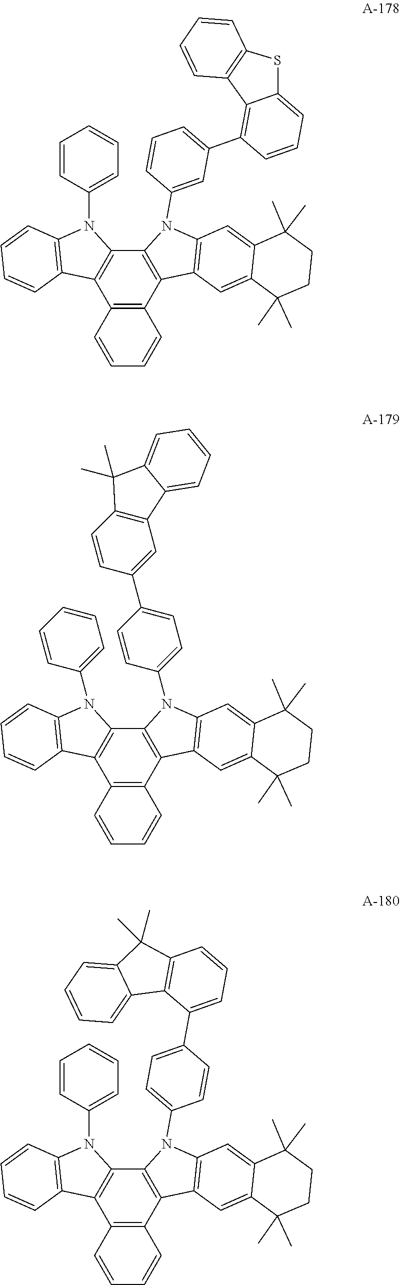 [SMILES: CC1(C)CCC(C)(C)c2cc3c(cc21)c1c2ccccc2c2c4ccccc4n(-c4ccccc4)c2c1n3-c1cccc(-c2cccc3sc4ccccc4c23)c1 ; CC1(C)CCC(C)(C)c2cc3c(cc21)c1c2ccccc2c2c4ccccc4n(-c4ccccc4)c2c1n3-c1ccc(-c2ccc3c(c2)-c2ccccc2C3(C)C)cc1 ; CC1(C)CCC(C)(C)c2cc3c(cc21)c1c2ccccc2c2c4ccccc4n(-c4ccccc4)c2c1n3-c1ccc(-c2cccc3c2-c2ccccc2C3(C)C)cc1]

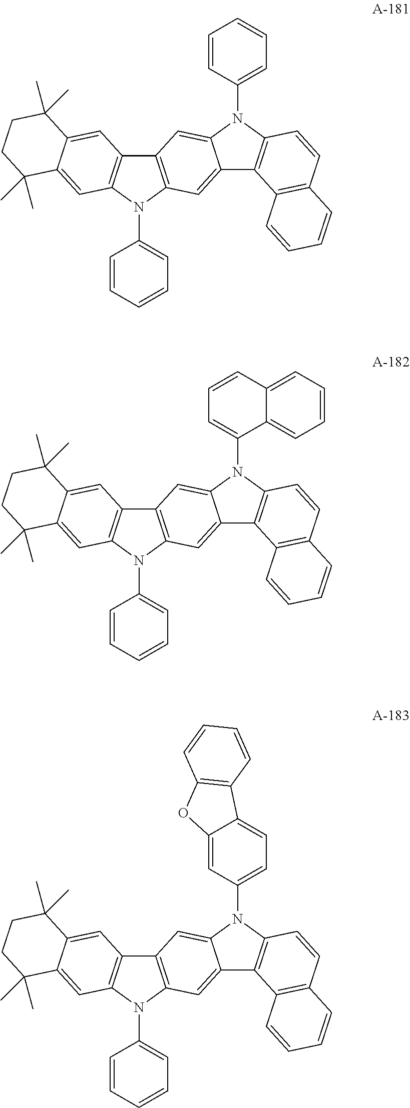 [SMILES: CC1(C)CCC(C)(C)c2cc3c(cc21)c1cc2c(cc1n3-c1ccccc1)c1c3ccccc3ccc1n2-c1ccccc1 ; CC1(C)CCC(C)(C)c2cc3c(cc21)c1cc2c(cc1n3-c1ccccc1)c1c3ccccc3ccc1n2-c1cccc2ccccc12 ; CC1(C)CCC(C)(C)c2cc3c(cc21)c1cc2c(cc1n3-c1ccccc1)c1c3ccccc3ccc1n2-c1ccc2c(c1)oc1ccccc12]

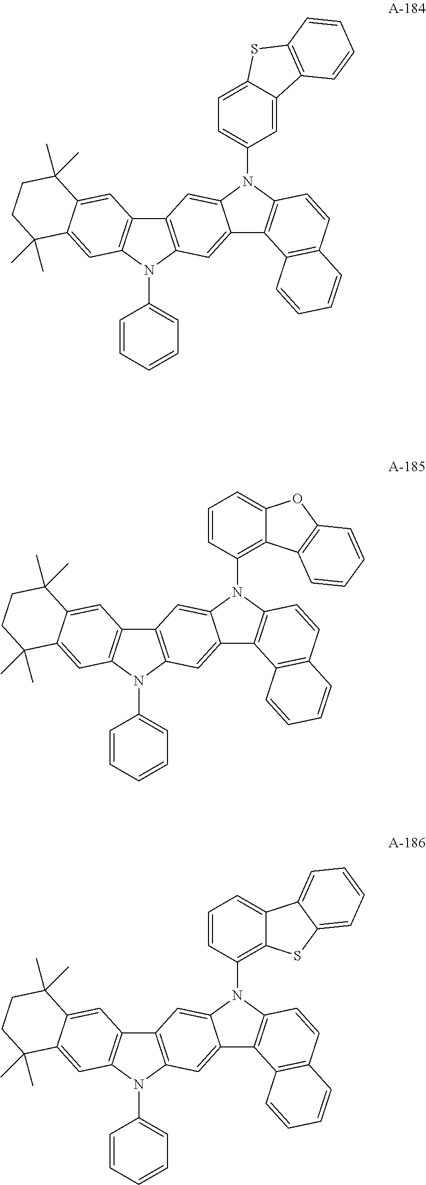 [SMILES: CC1(C)CCC(C)(C)c2cc3c(cc21)c1cc2c(cc1n3-c1ccccc1)c1c3ccccc3ccc1n2-c1ccc2sc3ccccc3c2c1 ; CC1(C)CCC(C)(C)c2cc3c(cc21)c1cc2c(cc1n3-c1ccccc1)c1c3ccccc3ccc1n2-c1cccc2oc3ccccc3c12 ; CC1(C)CCC(C)(C)c2cc3c(cc21)c1cc2c(cc1n3-c1ccccc1)c1c3ccccc3ccc1n2-c1cccc2c1sc1ccccc12]

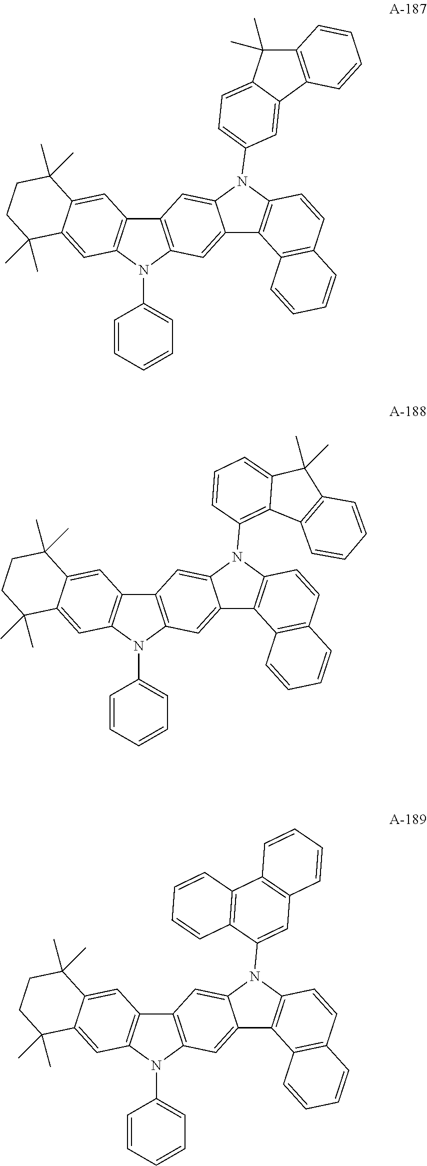 [SMILES: CC1(C)CCC(C)(C)c2cc3c(cc21)c1cc2c(cc1n3-c1ccccc1)c1c3ccccc3ccc1n2-c1ccc2c(c1)-c1ccccc1C2(C)C ; CC1(C)CCC(C)(C)c2cc3c(cc21)c1cc2c(cc1n3-c1ccccc1)c1c3ccccc3ccc1n2-c1cccc2c1-c1ccccc1C2(C)C ; CC1(C)CCC(C)(C)c2cc3c(cc21)c1cc2c(cc1n3-c1ccccc1)c1c3ccccc3ccc1n2-c1cc2ccccc2c2ccccc12]

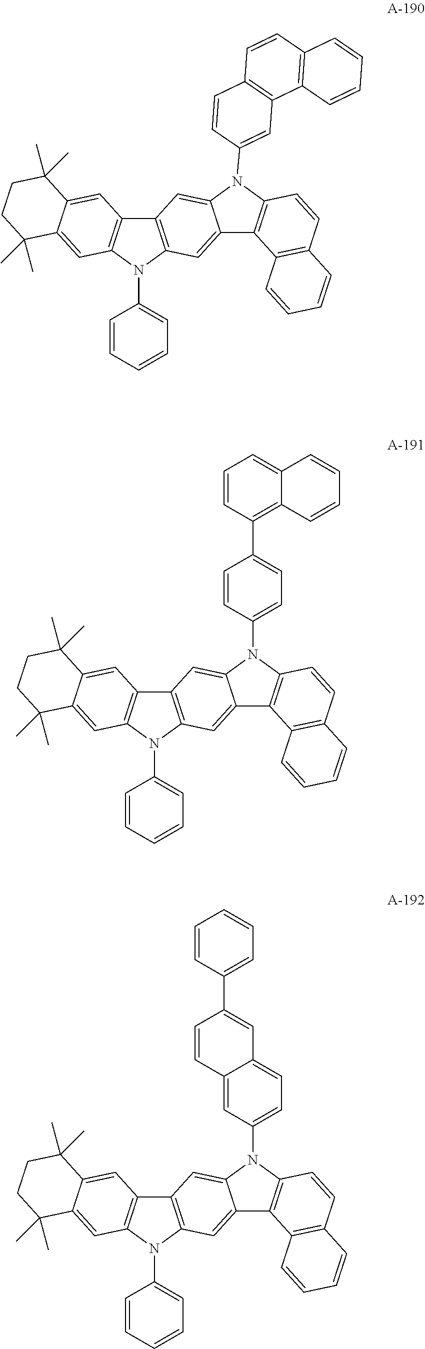 [SMILES: CC1(C)CCC(C)(C)c2cc3c(cc21)c1cc2c(cc1n3-c1ccccc1)c1c3ccccc3ccc1n2-c1ccc2ccc3ccccc3c2c1 ; CC1(C)CCC(C)(C)c2cc3c(cc21)c1cc2c(cc1n3-c1ccccc1)c1c3ccccc3ccc1n2-c1ccc(-c2cccc3ccccc23)cc1 ; CC1(C)CCC(C)(C)c2cc3c(cc21)c1cc2c(cc1n3-c1ccccc1)c1c3ccccc3ccc1n2-c1ccc2cc(-c3ccccc3)ccc2c1]

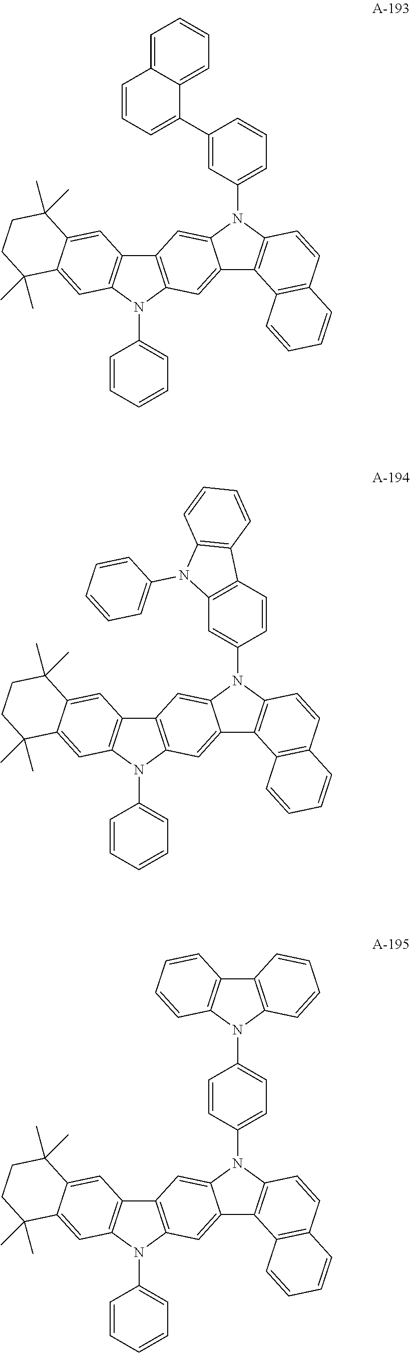 [SMILES: CC1(C)CCC(C)(C)c2cc3c(cc21)c1cc2c(cc1n3-c1ccccc1)c1c3ccccc3ccc1n2-c1cccc(-c2cccc3ccccc23)c1 ; CC1(C)CCC(C)(C)c2cc3c(cc21)c1cc2c(cc1n3-c1ccccc1)c1c3ccccc3ccc1n2-c1ccc2c3ccccc3n(-c3ccccc3)c2c1 ; CC1(C)CCC(C)(C)c2cc3c(cc21)c1cc2c(cc1n3-c1ccccc1)c1c3ccccc3ccc1n2-c1ccc(-n2c3ccccc3c3ccccc32)cc1]

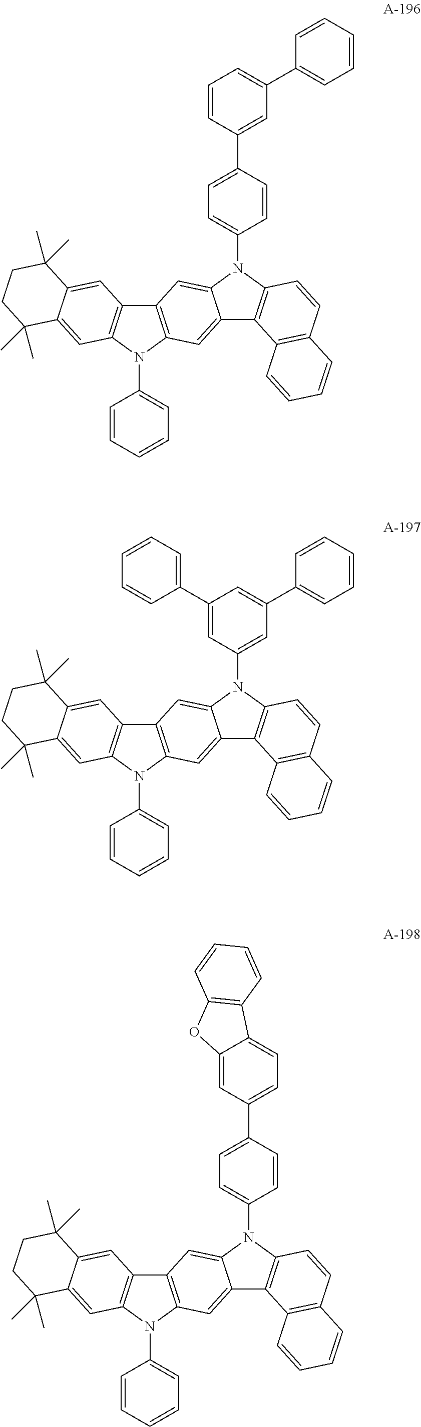 [SMILES: CC1(C)CCC(C)(C)c2cc3c(cc21)c1cc2c(cc1n3-c1ccccc1)c1c3ccccc3ccc1n2-c1ccc(-c2cccc(-c3ccccc3)c2)cc1 ; CC1(C)CCC(C)(C)c2cc3c(cc21)c1cc2c(cc1n3-c1ccccc1)c1c3ccccc3ccc1n2-c1cc(-c2ccccc2)cc(-c2ccccc2)c1 ; CC1(C)CCC(C)(C)c2cc3c(cc21)c1cc2c(cc1n3-c1ccccc1)c1c3ccccc3ccc1n2-c1ccc(-c2ccc3c(c2)oc2ccccc23)cc1]

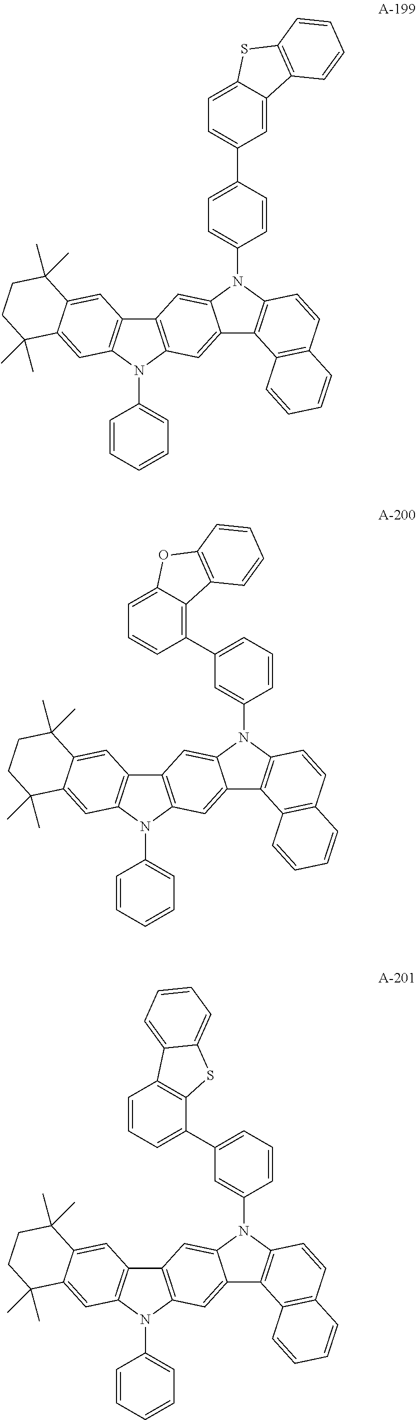 [SMILES: CC1(C)CCC(C)(C)c2cc3c(cc21)c1cc2c(cc1n3-c1ccccc1)c1c3ccccc3ccc1n2-c1ccc(-c2ccc3sc4ccccc4c3c2)cc1 ; CC1(C)CCC(C)(C)c2cc3c(cc21)c1cc2c(cc1n3-c1ccccc1)c1c3ccccc3ccc1n2-c1cccc(-c2cccc3oc4ccccc4c23)c1 ; CC1(C)CCC(C)(C)c2cc3c(cc21)c1cc2c(cc1n3-c1ccccc1)c1c3ccccc3ccc1n2-c1cccc(-c2cccc3c2sc2ccccc23)c1]

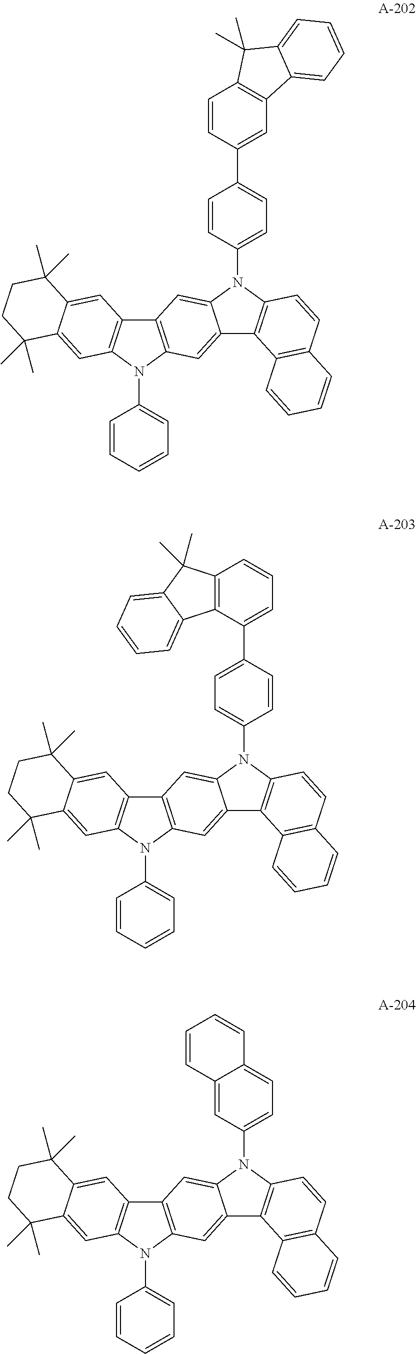 [SMILES: CC1(C)CCC(C)(C)c2cc3c(cc21)c1cc2c(cc1n3-c1ccccc1)c1c3ccccc3ccc1n2-c1ccc(-c2ccc3c(c2)-c2ccccc2C3(C)C)cc1 ; CC1(C)CCC(C)(C)c2cc3c(cc21)c1cc2c(cc1n3-c1ccccc1)c1c3ccccc3ccc1n2-c1ccc(-c2cccc3c2-c2ccccc2C3(C)C)cc1 ; CC1(C)CCC(C)(C)c2cc3c(cc21)c1cc2c(cc1n3-c1ccccc1)c1c3ccccc3ccc1n2-c1ccc2ccccc2c1]

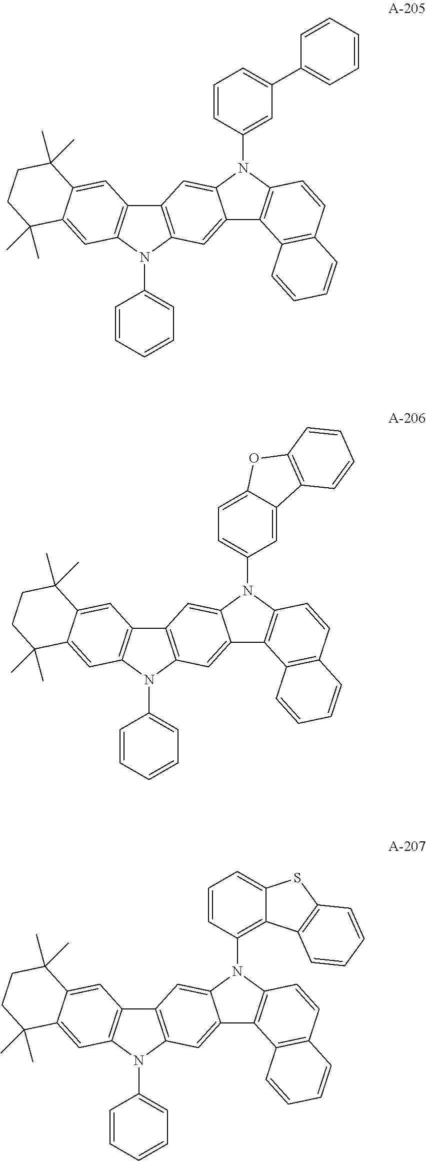 [SMILES: CC1(C)CCC(C)(C)c2cc3c(cc21)c1cc2c(cc1n3-c1ccccc1)c1c3ccccc3ccc1n2-c1cccc(-c2ccccc2)c1 ; CC1(C)CCC(C)(C)c2cc3c(cc21)c1cc2c(cc1n3-c1ccccc1)c1c3ccccc3ccc1n2-c1ccc2oc3ccccc3c2c1 ; CC1(C)CCC(C)(C)c2cc3c(cc21)c1cc2c(cc1n3-c1ccccc1)c1c3ccccc3ccc1n2-c1cccc2sc3ccccc3c12]

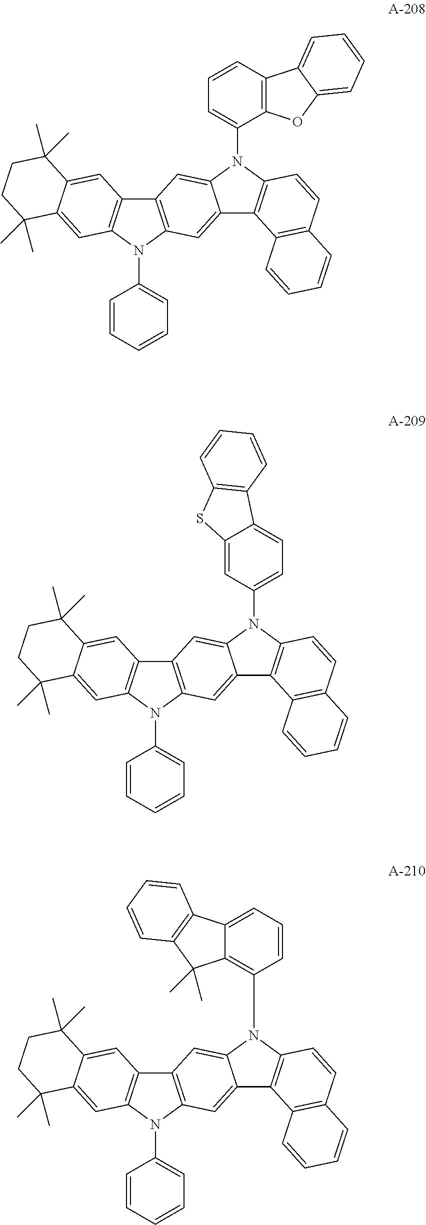 [SMILES: CC1(C)CCC(C)(C)c2cc3c(cc21)c1cc2c(cc1n3-c1ccccc1)c1c3ccccc3ccc1n2-c1cccc2c1oc1ccccc12 ; CC1(C)CCC(C)(C)c2cc3c(cc21)c1cc2c(cc1n3-c1ccccc1)c1c3ccccc3ccc1n2-c1ccc2c(c1)sc1ccccc12 ; CC1(C)CCC(C)(C)c2cc3c(cc21)c1cc2c(cc1n3-c1ccccc1)c1c3ccccc3ccc1n2-c1cccc2c1C(C)(C)c1ccccc1-2]

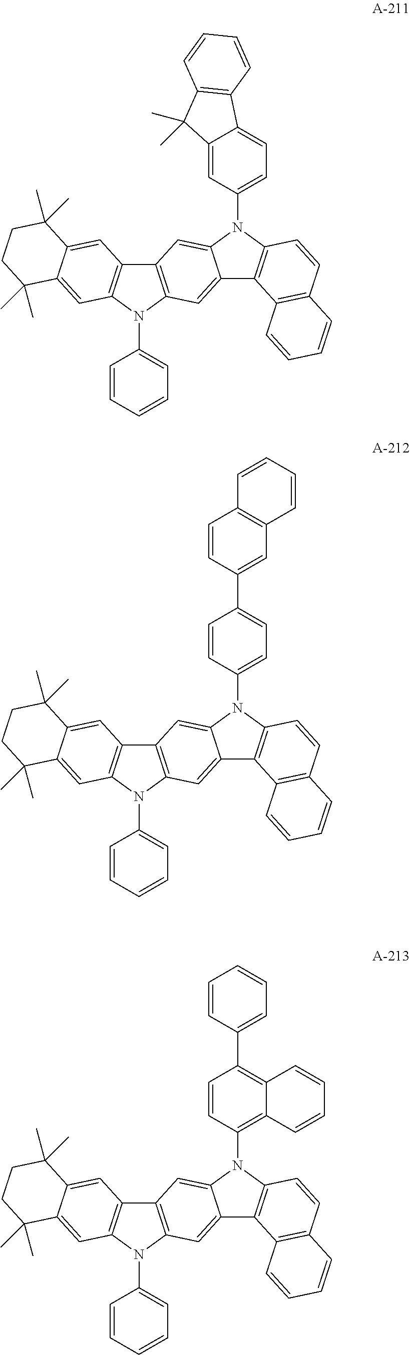 [SMILES: CC1(C)CCC(C)(C)c2cc3c(cc21)c1cc2c(cc1n3-c1ccccc1)c1c3ccccc3ccc1n2-c1ccc2c(c1)C(C)(C)c1ccccc1-2 ; CC1(C)CCC(C)(C)c2cc3c(cc21)c1cc2c(cc1n3-c1ccccc1)c1c3ccccc3ccc1n2-c1ccc(-c2ccc3ccccc3c2)cc1 ; CC1(C)CCC(C)(C)c2cc3c(cc21)c1cc2c(cc1n3-c1ccccc1)c1c3ccccc3ccc1n2-c1ccc(-c2ccccc2)c2ccccc12]

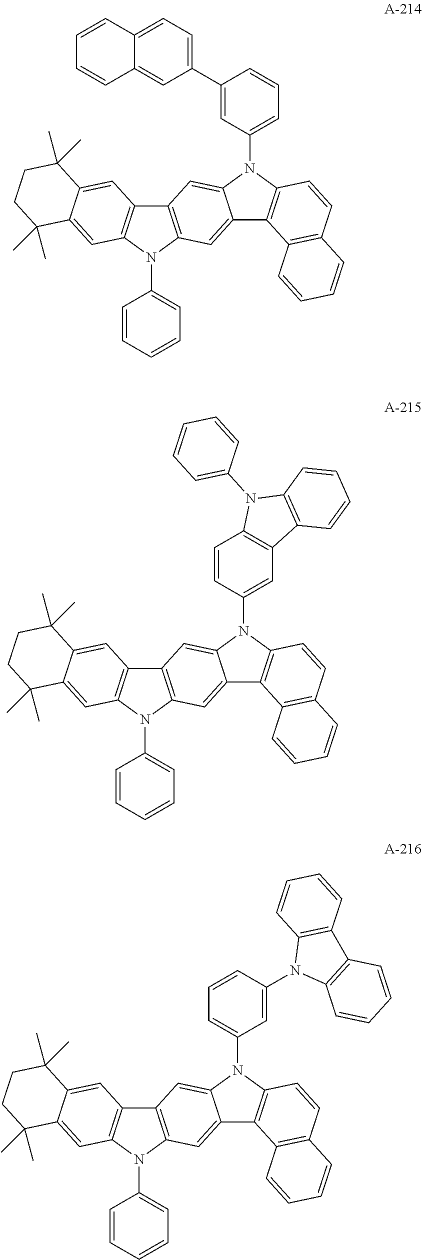 [SMILES: CC1(C)CCC(C)(C)c2cc3c(cc21)c1cc2c(cc1n3-c1ccccc1)c1c3ccccc3ccc1n2-c1cccc(-c2ccc3ccccc3c2)c1 ; CC1(C)CCC(C)(C)c2cc3c(cc21)c1cc2c(cc1n3-c1ccccc1)c1c3ccccc3ccc1n2-c1ccc2c(c1)c1ccccc1n2-c1ccccc1 ; CC1(C)CCC(C)(C)c2cc3c(cc21)c1cc2c(cc1n3-c1ccccc1)c1c3ccccc3ccc1n2-c1cccc(-n2c3ccccc3c3ccccc32)c1]

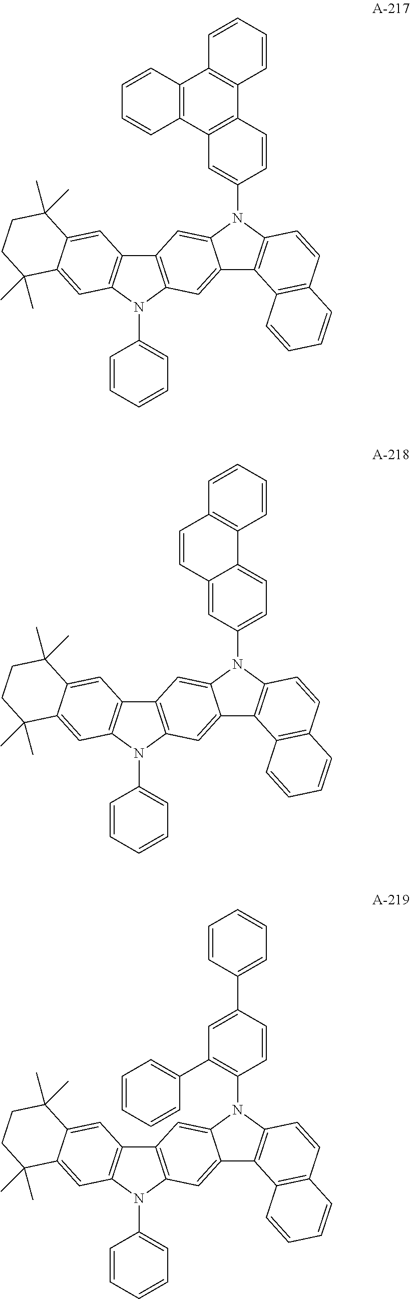 [SMILES: CC1(C)CCC(C)(C)c2cc3c(cc21)c1cc2c(cc1n3-c1ccccc1)c1c3ccccc3ccc1n2-c1ccc2c3ccccc3c3ccccc3c2c1 ; CC1(C)CCC(C)(C)c2cc3c(cc21)c1cc2c(cc1n3-c1ccccc1)c1c3ccccc3ccc1n2-c1ccc2c(ccc3ccccc32)c1 ; CC1(C)CCC(C)(C)c2cc3c(cc21)c1cc2c(cc1n3-c1ccccc1)c1c3ccccc3ccc1n2-c1ccc(-c2ccccc2)cc1-c1ccccc1]

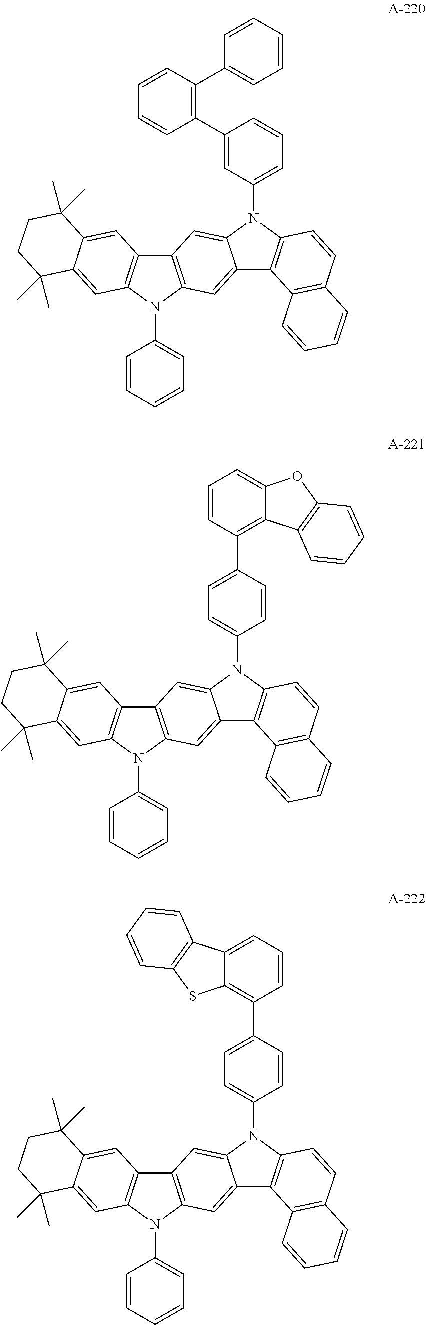 [SMILES: CC1(C)CCC(C)(C)c2cc3c(cc21)c1cc2c(cc1n3-c1ccccc1)c1c3ccccc3ccc1n2-c1cccc(-c2ccccc2-c2ccccc2)c1 ; CC1(C)CCC(C)(C)c2cc3c(cc21)c1cc2c(cc1n3-c1ccccc1)c1c3ccccc3ccc1n2-c1ccc(-c2cccc3oc4ccccc4c23)cc1 ; CC1(C)CCC(C)(C)c2cc3c(cc21)c1cc2c(cc1n3-c1ccccc1)c1c3ccccc3ccc1n2-c1ccc(-c2cccc3c2sc2ccccc23)cc1]

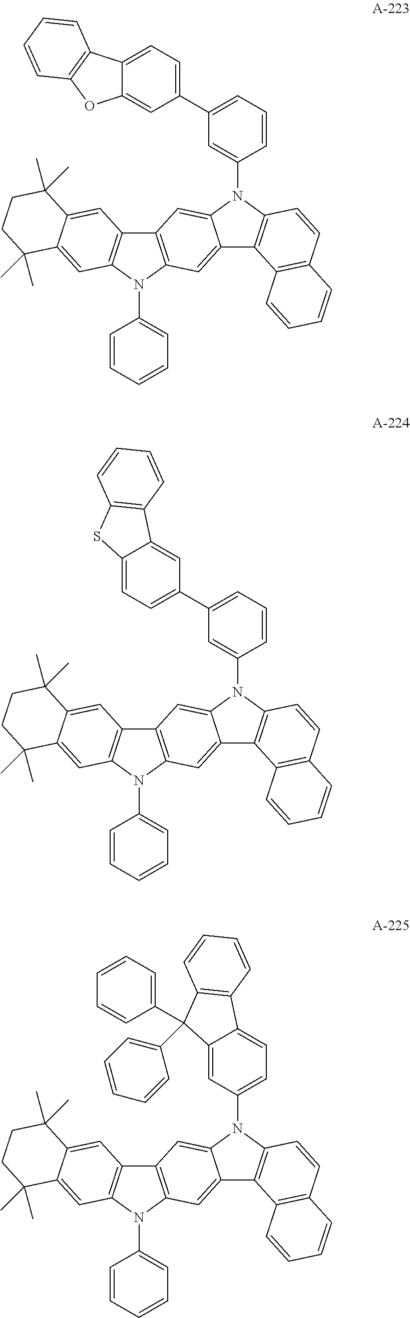 [SMILES: CC1(C)CCC(C)(C)c2cc3c(cc21)c1cc2c(cc1n3-c1ccccc1)c1c3ccccc3ccc1n2-c1cccc(-c2ccc3c(c2)oc2ccccc23)c1 ; CC1(C)CCC(C)(C)c2cc3c(cc21)c1cc2c(cc1n3-c1ccccc1)c1c3ccccc3ccc1n2-c1cccc(-c2ccc3sc4ccccc4c3c2)c1 ; CC1(C)CCC(C)(C)c2cc3c(cc21)c1cc2c(cc1n3-c1ccccc1)c1c3ccccc3ccc1n2-c1ccc2c(c1)C(c1ccccc1)(c1ccccc1)c1ccccc1-2]

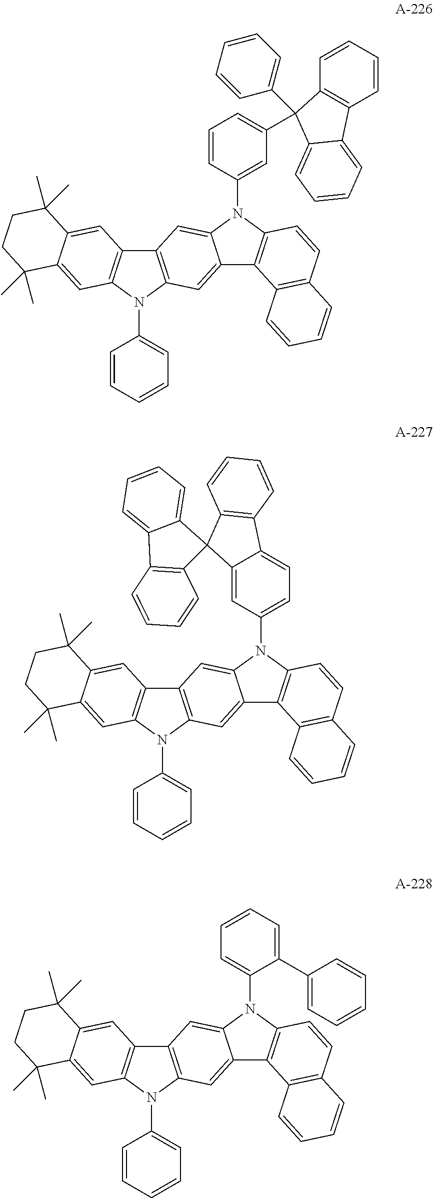 [SMILES: CC1(C)CCC(C)(C)c2cc3c(cc21)c1cc2c(cc1n3-c1ccccc1)c1c3ccccc3ccc1n2-c1cccc(C2(c3ccccc3)c3ccccc3-c3ccccc32)c1 ; CC1(C)CCC(C)(C)c2cc3c(cc21)c1cc2c(cc1n3-c1ccccc1)c1c3ccccc3ccc1n2-c1ccc2c(c1)C1(c3ccccc3-c3ccccc31)c1ccccc1-2 ; CC1(C)CCC(C)(C)c2cc3c(cc21)c1cc2c(cc1n3-c1ccccc1)c1c3ccccc3ccc1n2-c1ccccc1-c1ccccc1]

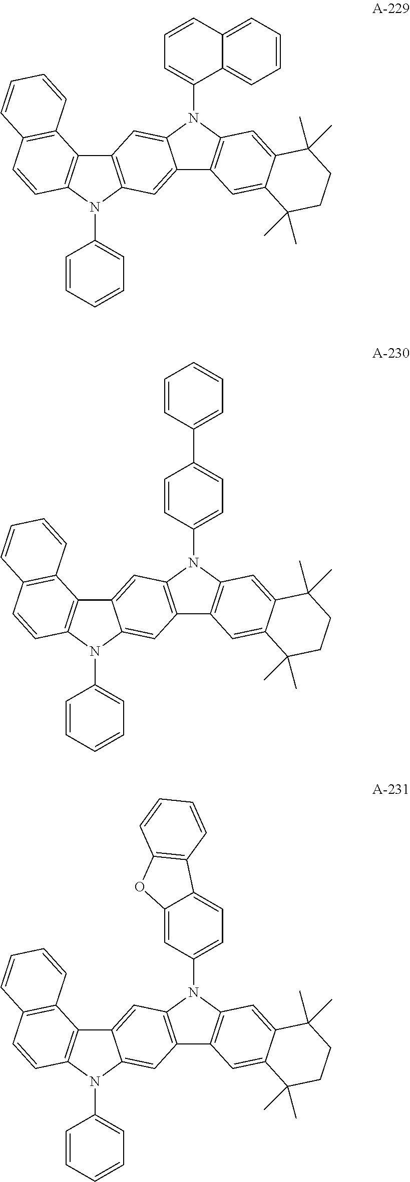 [SMILES: CC1(C)CCC(C)(C)c2cc3c(cc21)c1cc2c(cc1n3-c1cccc3ccccc13)c1c3ccccc3ccc1n2-c1ccccc1 ; CC1(C)CCC(C)(C)c2cc3c(cc21)c1cc2c(cc1n3-c1ccc(-c3ccccc3)cc1)c1c3ccccc3ccc1n2-c1ccccc1 ; CC1(C)CCC(C)(C)c2cc3c(cc21)c1cc2c(cc1n3-c1ccc3c(c1)oc1ccccc13)c1c3ccccc3ccc1n2-c1ccccc1]

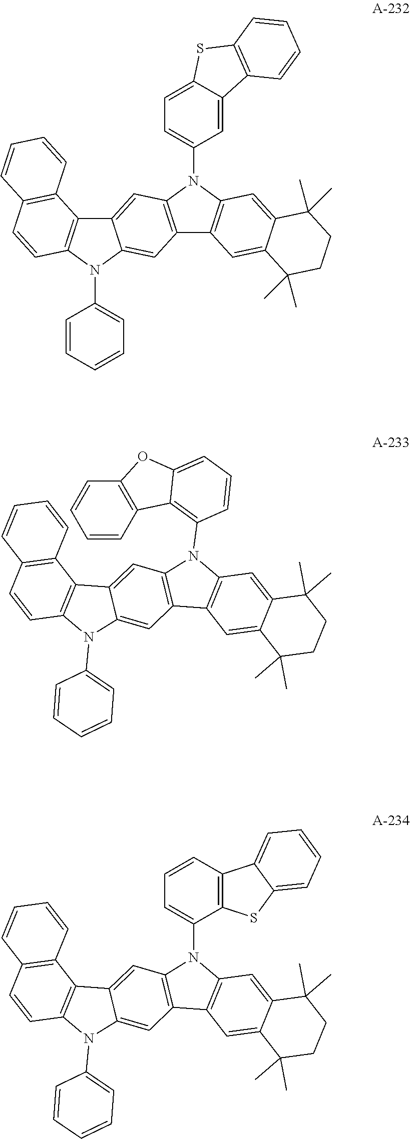 [SMILES: CC1(C)CCC(C)(C)c2cc3c(cc21)c1cc2c(cc1n3-c1ccc3sc4ccccc4c3c1)c1c3ccccc3ccc1n2-c1ccccc1 ; CC1(C)CCC(C)(C)c2cc3c(cc21)c1cc2c(cc1n3-c1cccc3oc4ccccc4c13)c1c3ccccc3ccc1n2-c1ccccc1 ; CC1(C)CCC(C)(C)c2cc3c(cc21)c1cc2c(cc1n3-c1cccc3c1sc1ccccc13)c1c3ccccc3ccc1n2-c1ccccc1]

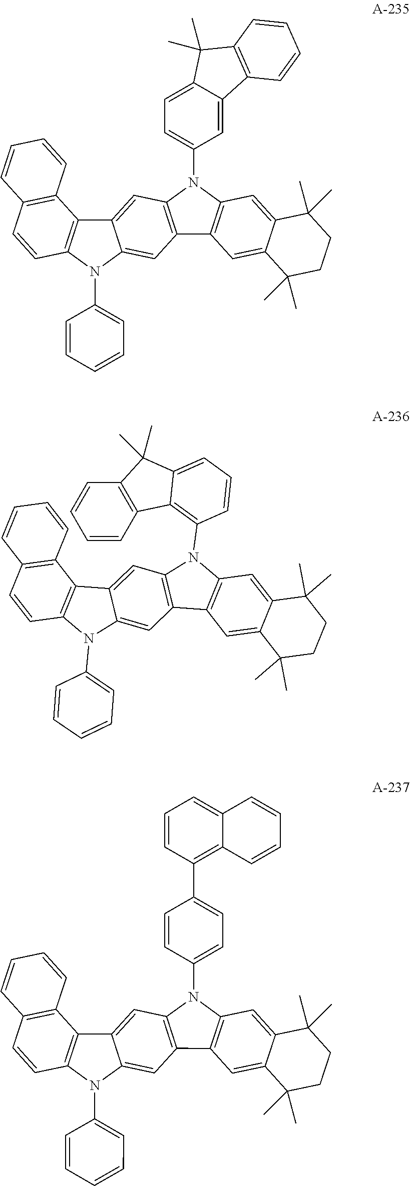 [SMILES: CC1(C)CCC(C)(C)c2cc3c(cc21)c1cc2c(cc1n3-c1ccc3c(c1)-c1ccccc1C3(C)C)c1c3ccccc3ccc1n2-c1ccccc1 ; CC1(C)CCC(C)(C)c2cc3c(cc21)c1cc2c(cc1n3-c1cccc3c1-c1ccccc1C3(C)C)c1c3ccccc3ccc1n2-c1ccccc1 ; CC1(C)CCC(C)(C)c2cc3c(cc21)c1cc2c(cc1n3-c1ccc(-c3cccc4ccccc34)cc1)c1c3ccccc3ccc1n2-c1ccccc1]

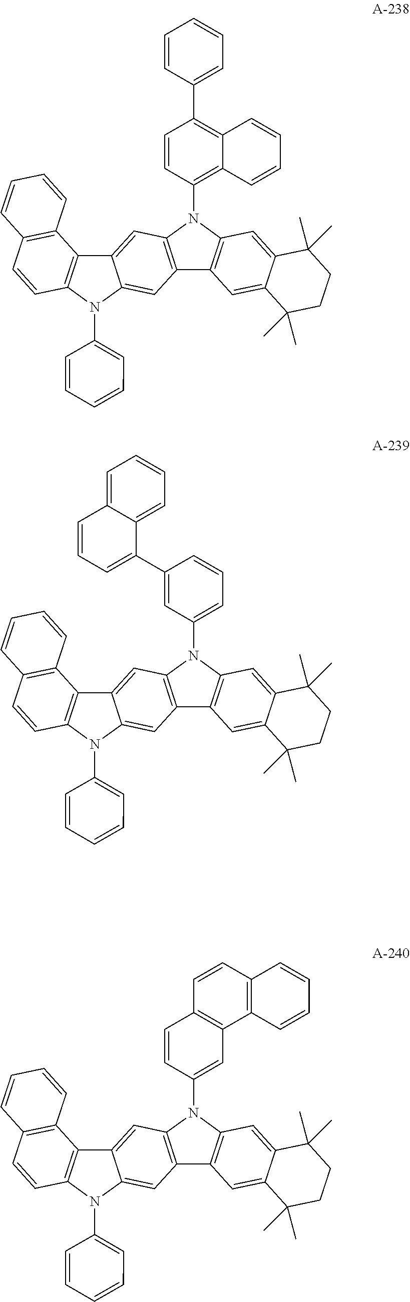 [SMILES: CC1(C)CCC(C)(C)c2cc3c(cc21)c1cc2c(cc1n3-c1ccc(-c3ccccc3)c3ccccc13)c1c3ccccc3ccc1n2-c1ccccc1 ; CC1(C)CCC(C)(C)c2cc3c(cc21)c1cc2c(cc1n3-c1cccc(-c3cccc4ccccc34)c1)c1c3ccccc3ccc1n2-c1ccccc1 ; CC1(C)CCC(C)(C)c2cc3c(cc21)c1cc2c(cc1n3-c1ccc3ccc4ccccc4c3c1)c1c3ccccc3ccc1n2-c1ccccc1]

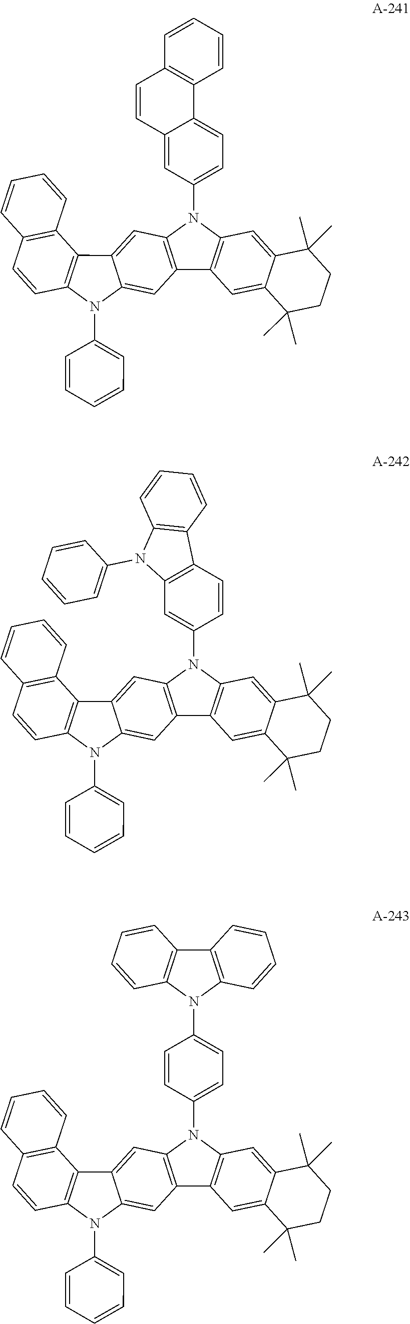 [SMILES: CC1(C)CCC(C)(C)c2cc3c(cc21)c1cc2c(cc1n3-c1ccc3c(ccc4ccccc43)c1)c1c3ccccc3ccc1n2-c1ccccc1 ; CC1(C)CCC(C)(C)c2cc3c(cc21)c1cc2c(cc1n3-c1ccc3c4ccccc4n(-c4ccccc4)c3c1)c1c3ccccc3ccc1n2-c1ccccc1 ; CC1(C)CCC(C)(C)c2cc3c(cc21)c1cc2c(cc1n3-c1ccc(-n3c4ccccc4c4ccccc43)cc1)c1c3ccccc3ccc1n2-c1ccccc1]

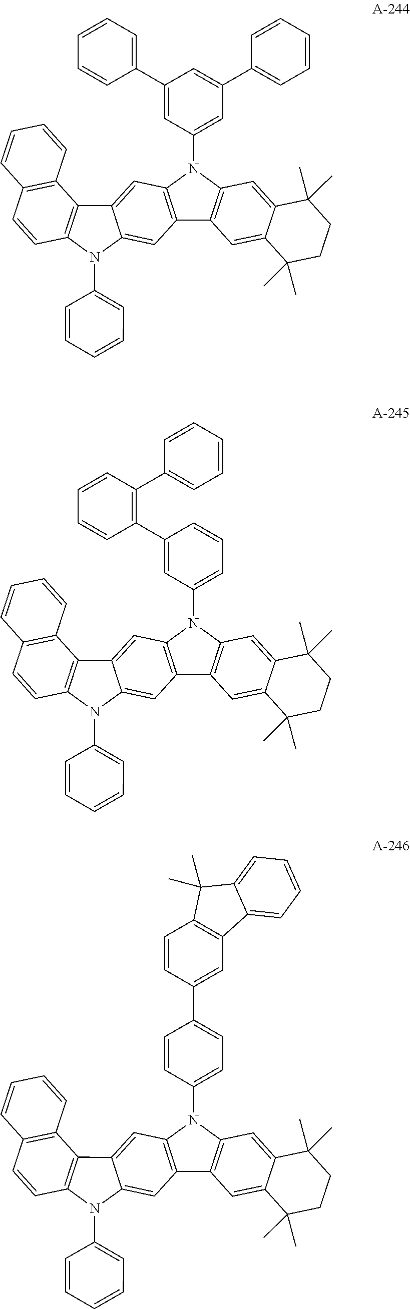 [SMILES: CC1(C)CCC(C)(C)c2cc3c(cc21)c1cc2c(cc1n3-c1cc(-c3ccccc3)cc(-c3ccccc3)c1)c1c3ccccc3ccc1n2-c1ccccc1 ; CC1(C)CCC(C)(C)c2cc3c(cc21)c1cc2c(cc1n3-c1cccc(-c3ccccc3-c3ccccc3)c1)c1c3ccccc3ccc1n2-c1ccccc1 ; CC1(C)CCC(C)(C)c2cc3c(cc21)c1cc2c(cc1n3-c1ccc(-c3ccc4c(c3)-c3ccccc3C4(C)C)cc1)c1c3ccccc3ccc1n2-c1ccccc1]

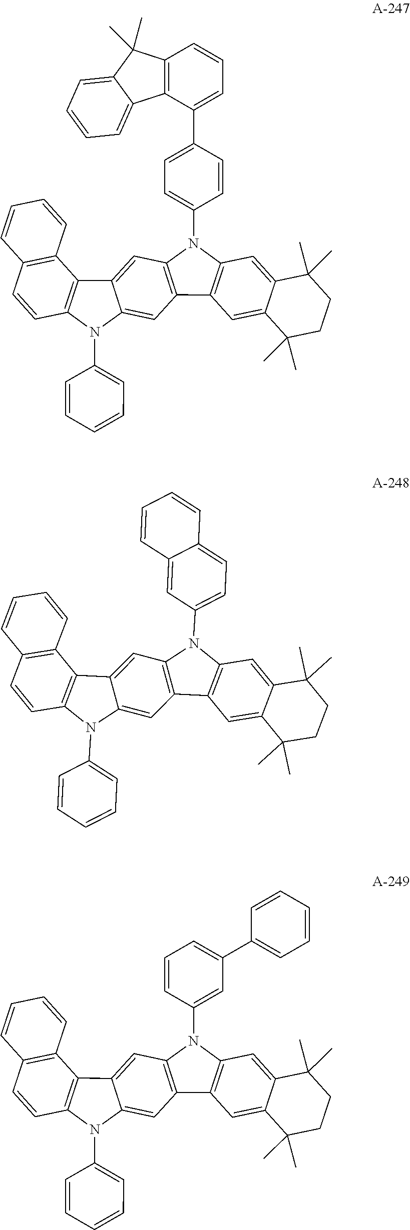 [SMILES: CC1(C)CCC(C)(C)c2cc3c(cc21)c1cc2c(cc1n3-c1ccc(-c3cccc4c3-c3ccccc3C4(C)C)cc1)c1c3ccccc3ccc1n2-c1ccccc1 ; CC1(C)CCC(C)(C)c2cc3c(cc21)c1cc2c(cc1n3-c1ccc3ccccc3c1)c1c3ccccc3ccc1n2-c1ccccc1 ; CC1(C)CCC(C)(C)c2cc3c(cc21)c1cc2c(cc1n3-c1cccc(-c3ccccc3)c1)c1c3ccccc3ccc1n2-c1ccccc1]

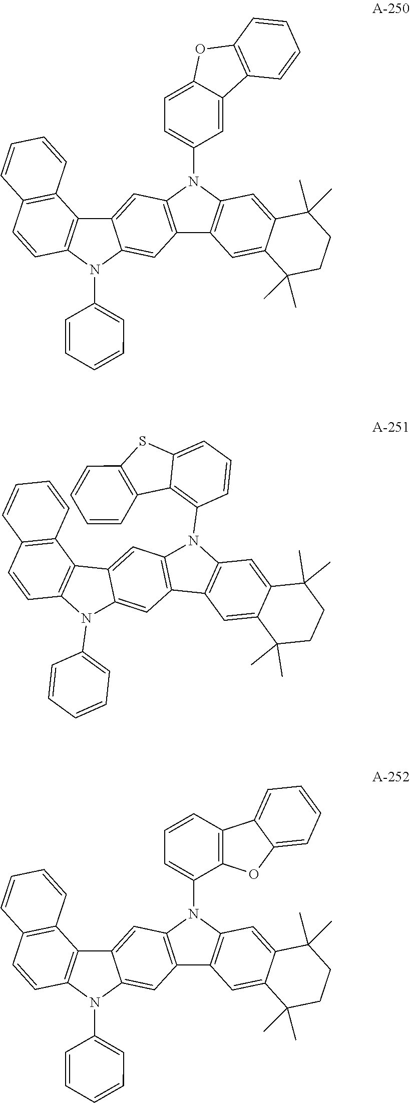 [SMILES: CC1(C)CCC(C)(C)c2cc3c(cc21)c1cc2c(cc1n3-c1ccc3oc4ccccc4c3c1)c1c3ccccc3ccc1n2-c1ccccc1 ; CC1(C)CCC(C)(C)c2cc3c(cc21)c1cc2c(cc1n3-c1cccc3sc4ccccc4c13)c1c3ccccc3ccc1n2-c1ccccc1 ; CC1(C)CCC(C)(C)c2cc3c(cc21)c1cc2c(cc1n3-c1cccc3c1oc1ccccc13)c1c3ccccc3ccc1n2-c1ccccc1]

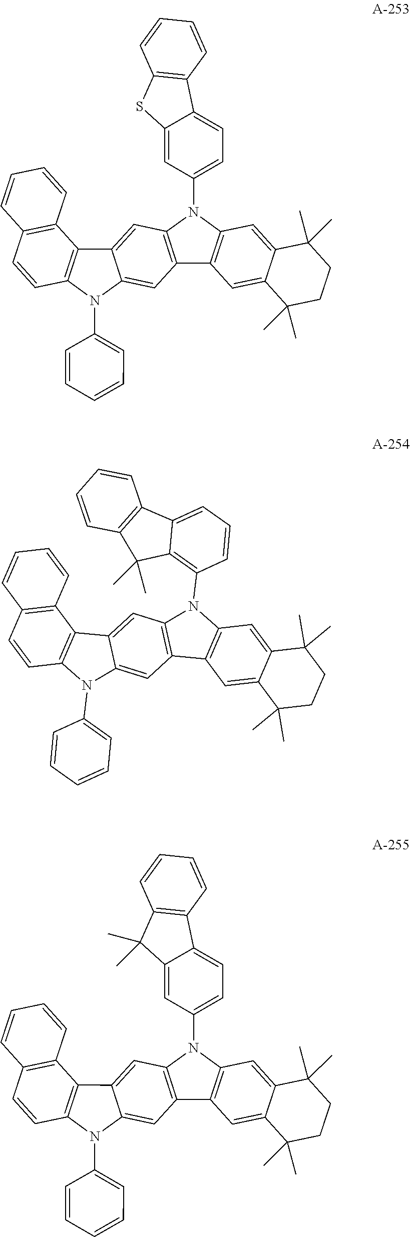 [SMILES: CC1(C)CCC(C)(C)c2cc3c(cc21)c1cc2c(cc1n3-c1ccc3c(c1)sc1ccccc13)c1c3ccccc3ccc1n2-c1ccccc1 ; CC1(C)CCC(C)(C)c2cc3c(cc21)c1cc2c(cc1n3-c1cccc3c1C(C)(C)c1ccccc1-3)c1c3ccccc3ccc1n2-c1ccccc1 ; CC1(C)CCC(C)(C)c2cc3c(cc21)c1cc2c(cc1n3-c1ccc3c(c1)C(C)(C)c1ccccc1-3)c1c3ccccc3ccc1n2-c1ccccc1]

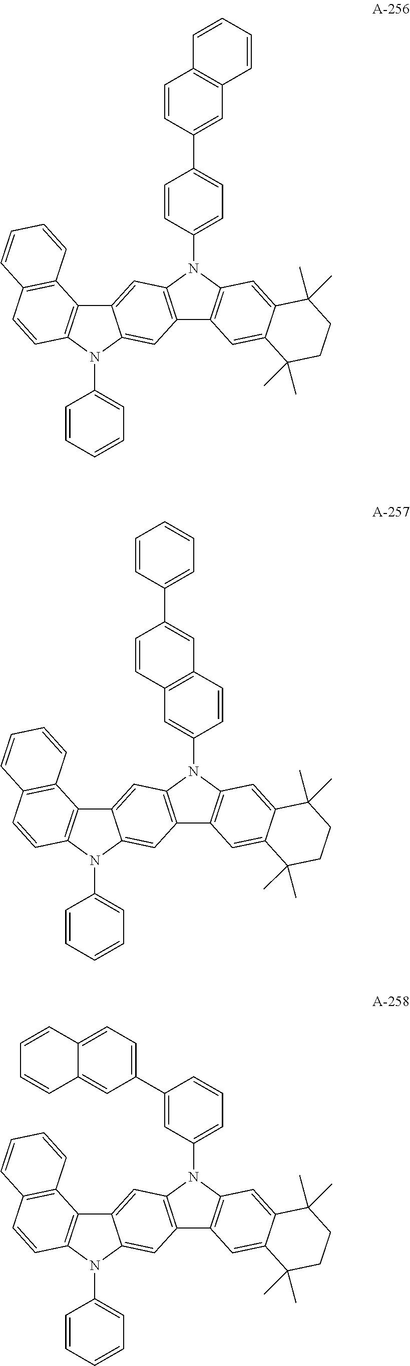 [SMILES: CC1(C)CCC(C)(C)c2cc3c(cc21)c1cc2c(cc1n3-c1ccc(-c3ccc4ccccc4c3)cc1)c1c3ccccc3ccc1n2-c1ccccc1 ; CC1(C)CCC(C)(C)c2cc3c(cc21)c1cc2c(cc1n3-c1ccc3cc(-c4ccccc4)ccc3c1)c1c3ccccc3ccc1n2-c1ccccc1 ; CC1(C)CCC(C)(C)c2cc3c(cc21)c1cc2c(cc1n3-c1cccc(-c3ccc4ccccc4c3)c1)c1c3ccccc3ccc1n2-c1ccccc1]

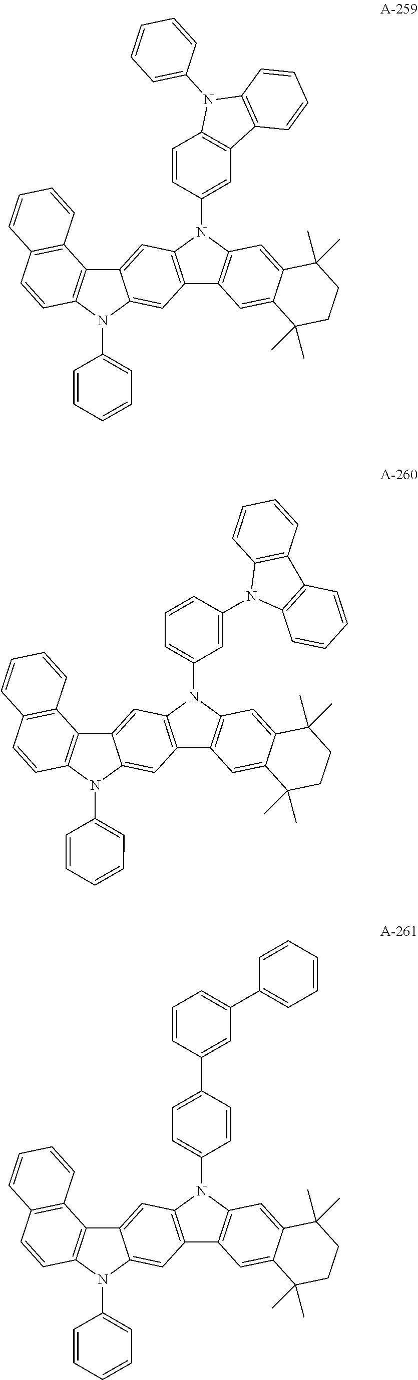 [SMILES: CC1(C)CCC(C)(C)c2cc3c(cc21)c1cc2c(cc1n3-c1ccc3c(c1)c1ccccc1n3-c1ccccc1)c1c3ccccc3ccc1n2-c1ccccc1 ; CC1(C)CCC(C)(C)c2cc3c(cc21)c1cc2c(cc1n3-c1cccc(-n3c4ccccc4c4ccccc43)c1)c1c3ccccc3ccc1n2-c1ccccc1 ; CC1(C)CCC(C)(C)c2cc3c(cc21)c1cc2c(cc1n3-c1ccc(-c3cccc(-c4ccccc4)c3)cc1)c1c3ccccc3ccc1n2-c1ccccc1]

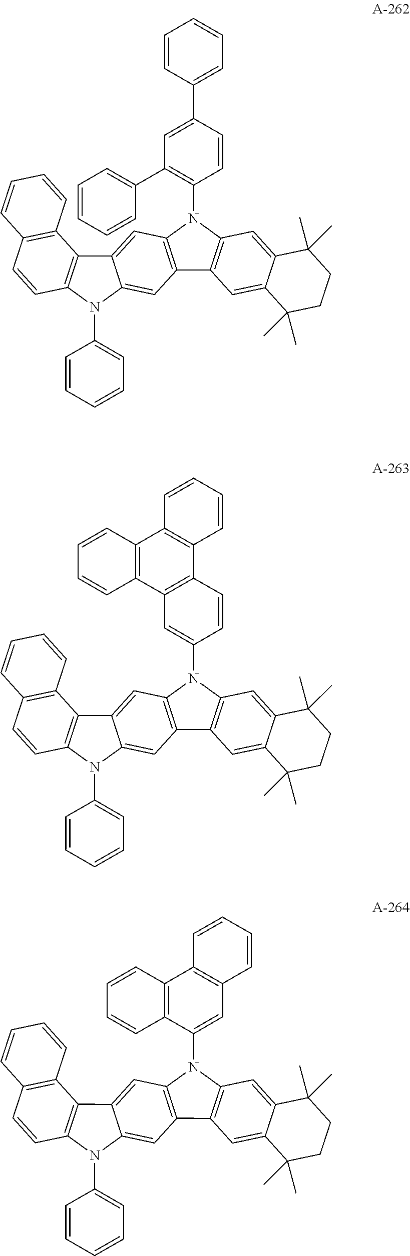 [SMILES: CC1(C)CCC(C)(C)c2cc3c(cc21)c1cc2c(cc1n3-c1ccc(-c3ccccc3)cc1-c1ccccc1)c1c3ccccc3ccc1n2-c1ccccc1 ; CC1(C)CCC(C)(C)c2cc3c(cc21)c1cc2c(cc1n3-c1ccc3c4ccccc4c4ccccc4c3c1)c1c3ccccc3ccc1n2-c1ccccc1 ; CC1(C)CCC(C)(C)c2cc3c(cc21)c1cc2c(cc1n3-c1cc3ccccc3c3ccccc13)c1c3ccccc3ccc1n2-c1ccccc1]

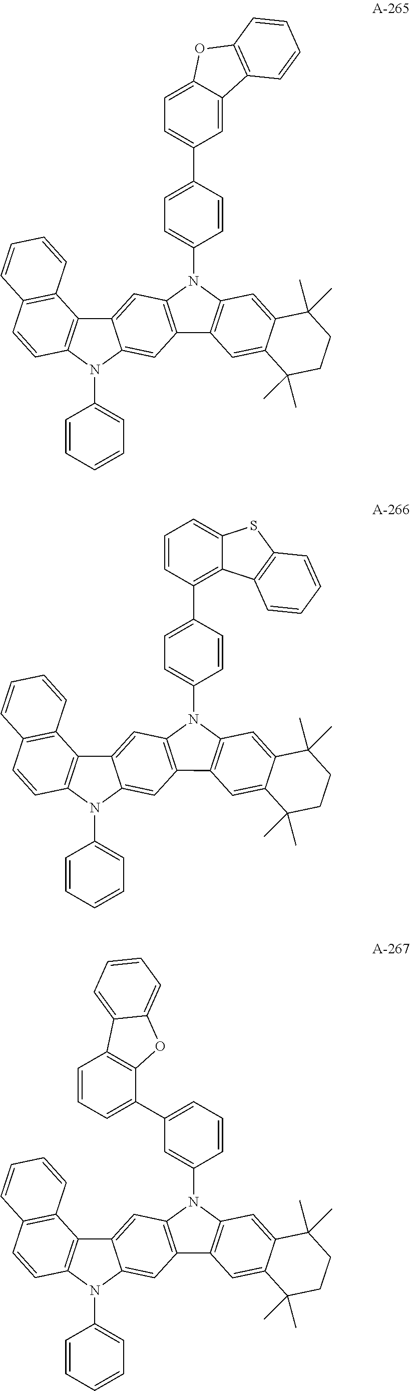 [SMILES: CC1(C)CCC(C)(C)c2cc3c(cc21)c1cc2c(cc1n3-c1ccc(-c3ccc4oc5ccccc5c4c3)cc1)c1c3ccccc3ccc1n2-c1ccccc1 ; CC1(C)CCC(C)(C)c2cc3c(cc21)c1cc2c(cc1n3-c1ccc(-c3cccc4sc5ccccc5c34)cc1)c1c3ccccc3ccc1n2-c1ccccc1 ; CC1(C)CCC(C)(C)c2cc3c(cc21)c1cc2c(cc1n3-c1cccc(-c3cccc4c3oc3ccccc34)c1)c1c3ccccc3ccc1n2-c1ccccc1]

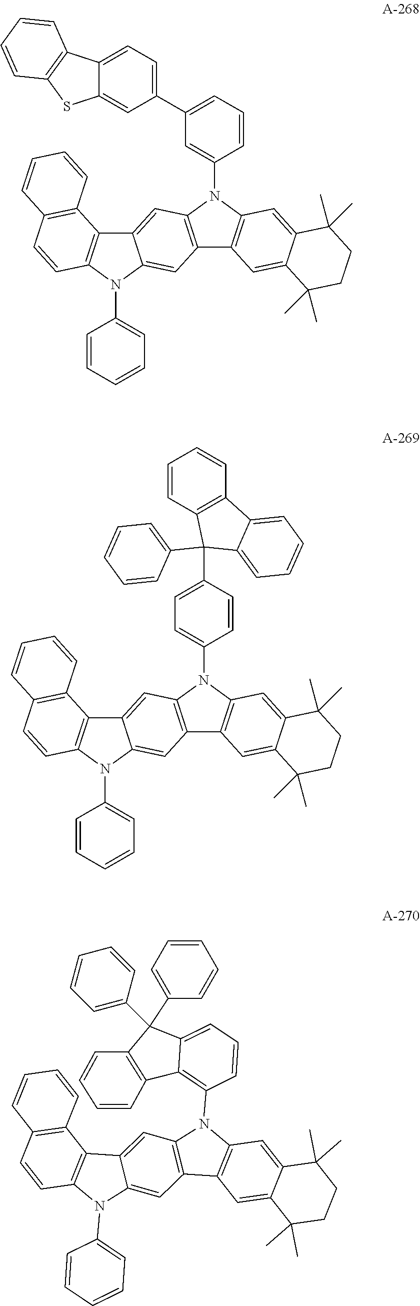 [SMILES: CC1(C)CCC(C)(C)c2cc3c(cc21)c1cc2c(cc1n3-c1cccc(-c3ccc4c(c3)sc3ccccc34)c1)c1c3ccccc3ccc1n2-c1ccccc1 ; CC1(C)CCC(C)(C)c2cc3c(cc21)c1cc2c(cc1n3-c1ccc(C3(c4ccccc4)c4ccccc4-c4ccccc43)cc1)c1c3ccccc3ccc1n2-c1ccccc1 ; CC1(C)CCC(C)(C)c2cc3c(cc21)c1cc2c(cc1n3-c1cccc3c1-c1ccccc1C3(c1ccccc1)c1ccccc1)c1c3ccccc3ccc1n2-c1ccccc1]

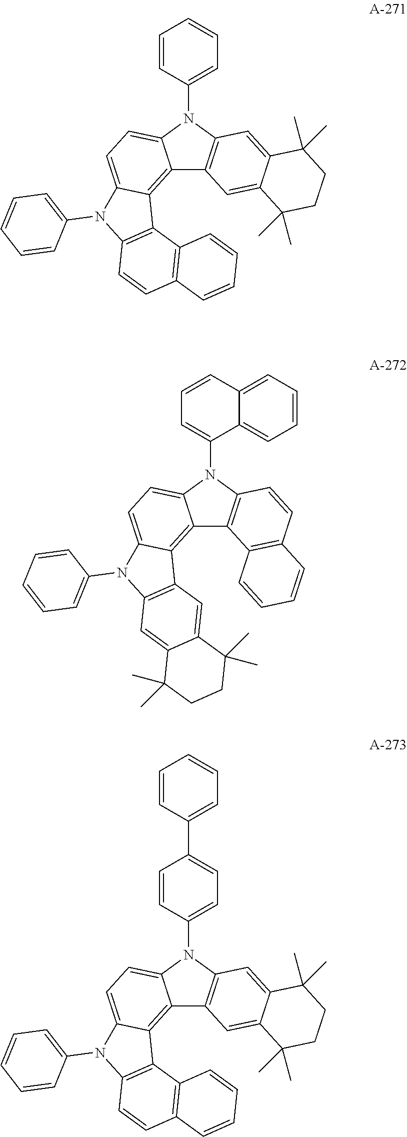 [SMILES: CC1(C)CCC(C)(C)c2cc3c(cc21)c1c2c4c5ccccc5ccc4n(-c4ccccc4)c2ccc1n3-c1ccccc1 ; CC1(C)CCC(C)(C)c2cc3c(cc21)c1c2c4c5ccccc5ccc4n(-c4cccc5ccccc45)c2ccc1n3-c1ccccc1 ; CC1(C)CCC(C)(C)c2cc3c(cc21)c1c2c4c5ccccc5ccc4n(-c4ccccc4)c2ccc1n3-c1ccc(-c2ccccc2)cc1]

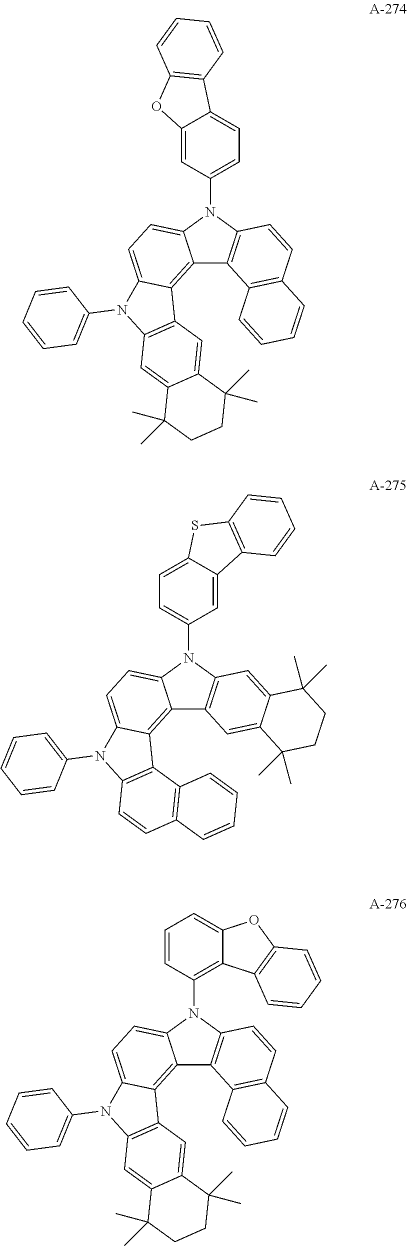 [SMILES: CC1(C)CCC(C)(C)c2cc3c(cc21)c1c2c4c5ccccc5ccc4n(-c4ccc5c(c4)oc4ccccc45)c2ccc1n3-c1ccccc1 ; CC1(C)CCC(C)(C)c2cc3c(cc21)c1c2c4c5ccccc5ccc4n(-c4ccccc4)c2ccc1n3-c1ccc2sc3ccccc3c2c1 ; CC1(C)CCC(C)(C)c2cc3c(cc21)c1c2c4c5ccccc5ccc4n(-c4cccc5oc6ccccc6c45)c2ccc1n3-c1ccccc1]

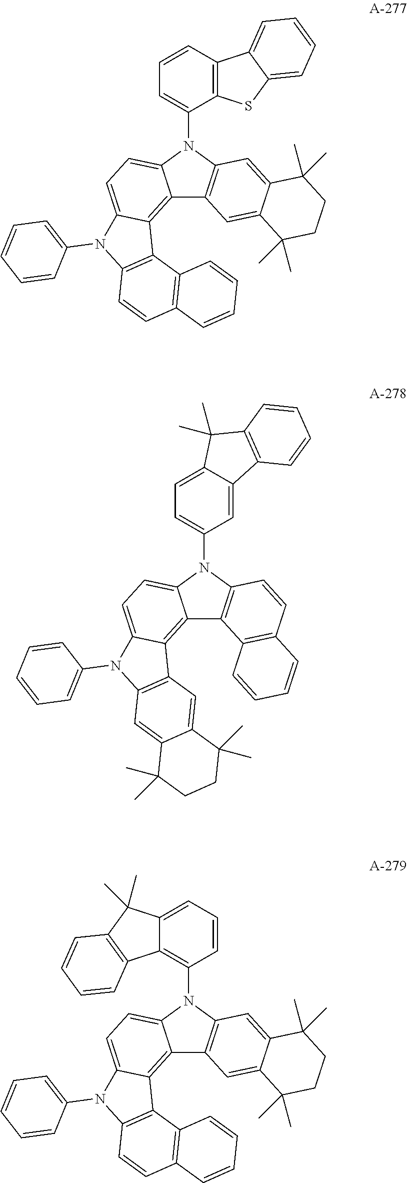 [SMILES: CC1(C)CCC(C)(C)c2cc3c(cc21)c1c2c4c5ccccc5ccc4n(-c4ccccc4)c2ccc1n3-c1cccc2c1sc1ccccc12 ; CC1(C)CCC(C)(C)c2cc3c(cc21)c1c2c4c5ccccc5ccc4n(-c4ccc5c(c4)-c4ccccc4C5(C)C)c2ccc1n3-c1ccccc1 ; CC1(C)CCC(C)(C)c2cc3c(cc21)c1c2c4c5ccccc5ccc4n(-c4ccccc4)c2ccc1n3-c1cccc2c1-c1ccccc1C2(C)C]

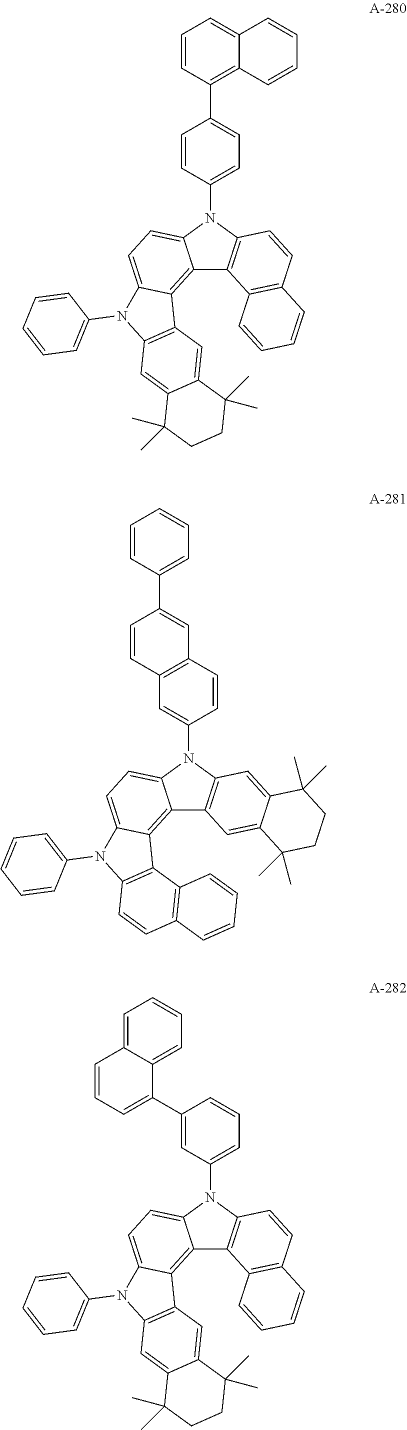 [SMILES: CC1(C)CCC(C)(C)c2cc3c(cc21)c1c2c4c5ccccc5ccc4n(-c4ccc(-c5cccc6ccccc56)cc4)c2ccc1n3-c1ccccc1 ; CC1(C)CCC(C)(C)c2cc3c(cc21)c1c2c4c5ccccc5ccc4n(-c4ccccc4)c2ccc1n3-c1ccc2cc(-c3ccccc3)ccc2c1 ; CC1(C)CCC(C)(C)c2cc3c(cc21)c1c2c4c5ccccc5ccc4n(-c4cccc(-c5cccc6ccccc56)c4)c2ccc1n3-c1ccccc1]

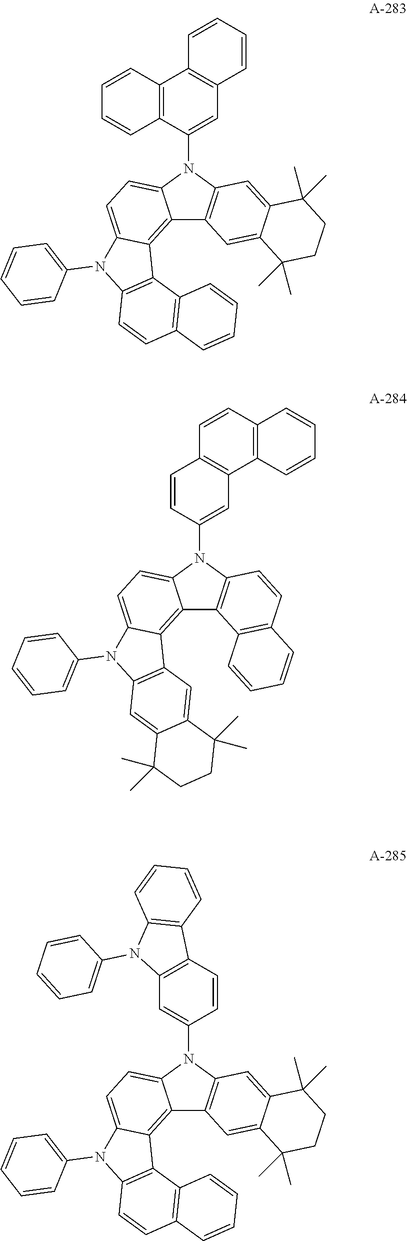 [SMILES: CC1(C)CCC(C)(C)c2cc3c(cc21)c1c2c4c5ccccc5ccc4n(-c4ccccc4)c2ccc1n3-c1cc2ccccc2c2ccccc12 ; CC1(C)CCC(C)(C)c2cc3c(cc21)c1c2c4c5ccccc5ccc4n(-c4ccc5ccc6ccccc6c5c4)c2ccc1n3-c1ccccc1 ; CC1(C)CCC(C)(C)c2cc3c(cc21)c1c2c4c5ccccc5ccc4n(-c4ccccc4)c2ccc1n3-c1ccc2c3ccccc3n(-c3ccccc3)c2c1]

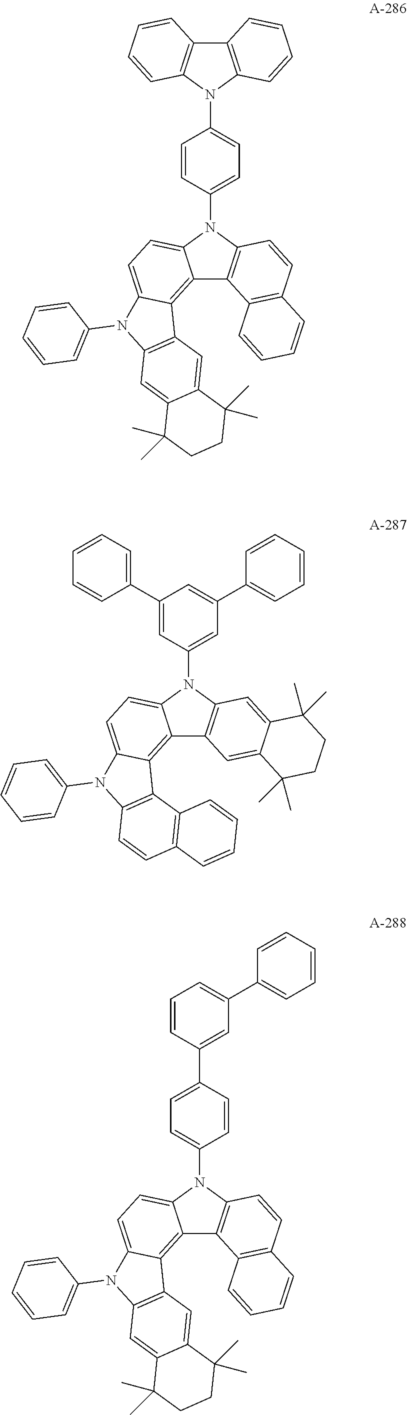 [SMILES: CC1(C)CCC(C)(C)c2cc3c(cc21)c1c2c4c5ccccc5ccc4n(-c4ccc(-n5c6ccccc6c6ccccc65)cc4)c2ccc1n3-c1ccccc1 ; CC1(C)CCC(C)(C)c2cc3c(cc21)c1c2c4c5ccccc5ccc4n(-c4ccccc4)c2ccc1n3-c1cc(-c2ccccc2)cc(-c2ccccc2)c1 ; CC1(C)CCC(C)(C)c2cc3c(cc21)c1c2c4c5ccccc5ccc4n(-c4ccc(-c5cccc(-c6ccccc6)c5)cc4)c2ccc1n3-c1ccccc1]

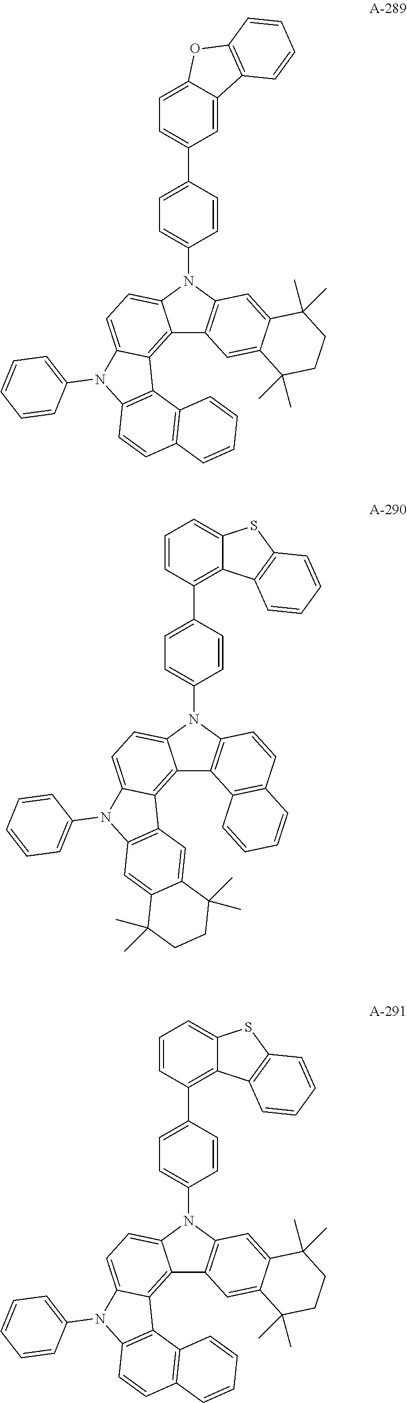 [SMILES: CC1(C)CCC(C)(C)c2cc3c(cc21)c1c2c4c5ccccc5ccc4n(-c4ccccc4)c2ccc1n3-c1ccc(-c2ccc3oc4ccccc4c3c2)cc1 ; CC1(C)CCC(C)(C)c2cc3c(cc21)c1c2c4c5ccccc5ccc4n(-c4ccc(-c5cccc6sc7ccccc7c56)cc4)c2ccc1n3-c1ccccc1 ; CC1(C)CCC(C)(C)c2cc3c(cc21)c1c2c4c5ccccc5ccc4n(-c4ccccc4)c2ccc1n3-c1ccc(-c2cccc3sc4ccccc4c23)cc1]

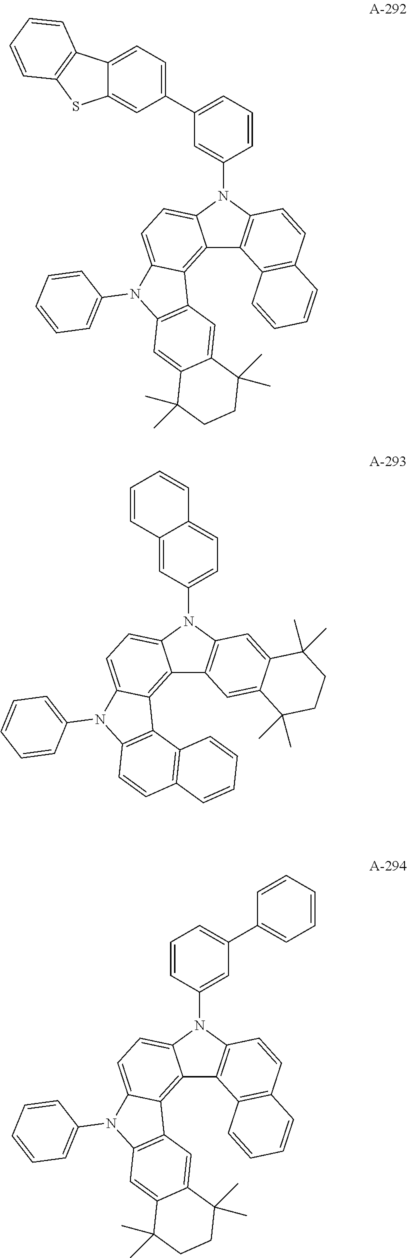

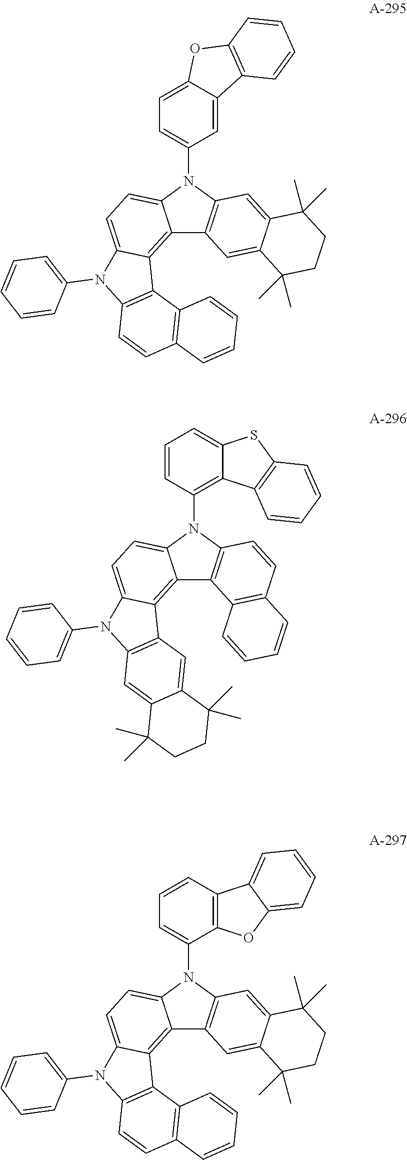 [SMILES: CC1(C)CCC(C)(C)c2cc3c(cc21)c1c2c4c5ccccc5ccc4n(-c4ccccc4)c2ccc1n3-c1ccc2oc3ccccc3c2c1 ; CC1(C)CCC(C)(C)c2cc3c(cc21)c1c2c4c5ccccc5ccc4n(-c4cccc5sc6ccccc6c45)c2ccc1n3-c1ccccc1 ; CC1(C)CCC(C)(C)c2cc3c(cc21)c1c2c4c5ccccc5ccc4n(-c4ccccc4)c2ccc1n3-c1cccc2c1oc1ccccc12]

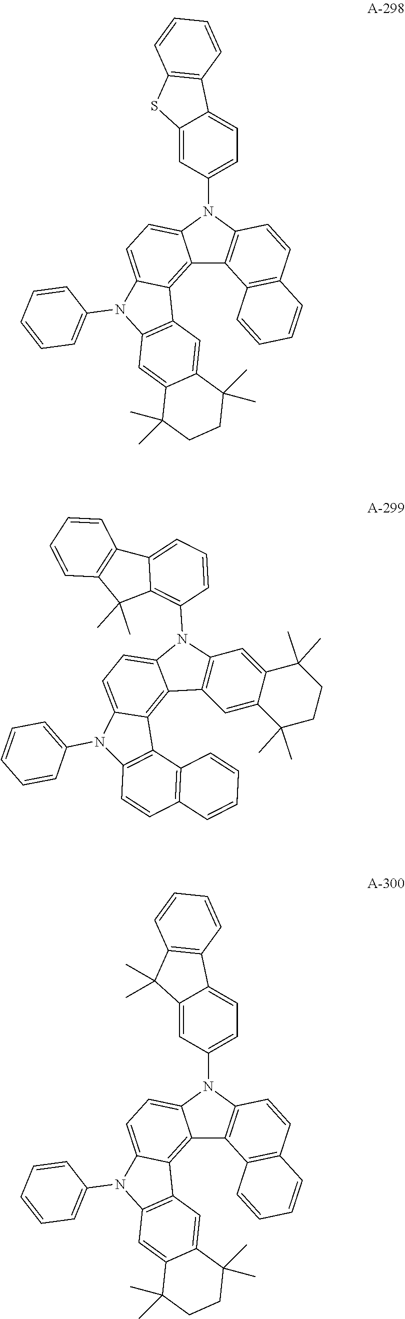 [SMILES: CC1(C)CCC(C)(C)c2cc3c(cc21)c1c2c4c5ccccc5ccc4n(-c4ccc5c(c4)sc4ccccc45)c2ccc1n3-c1ccccc1 ; CC1(C)CCC(C)(C)c2cc3c(cc21)c1c2c4c5ccccc5ccc4n(-c4ccccc4)c2ccc1n3-c1cccc2c1C(C)(C)c1ccccc1-2 ; CC1(C)CCC(C)(C)c2cc3c(cc21)c1c2c4c5ccccc5ccc4n(-c4ccc5c(c4)C(C)(C)c4ccccc4-5)c2ccc1n3-c1ccccc1]

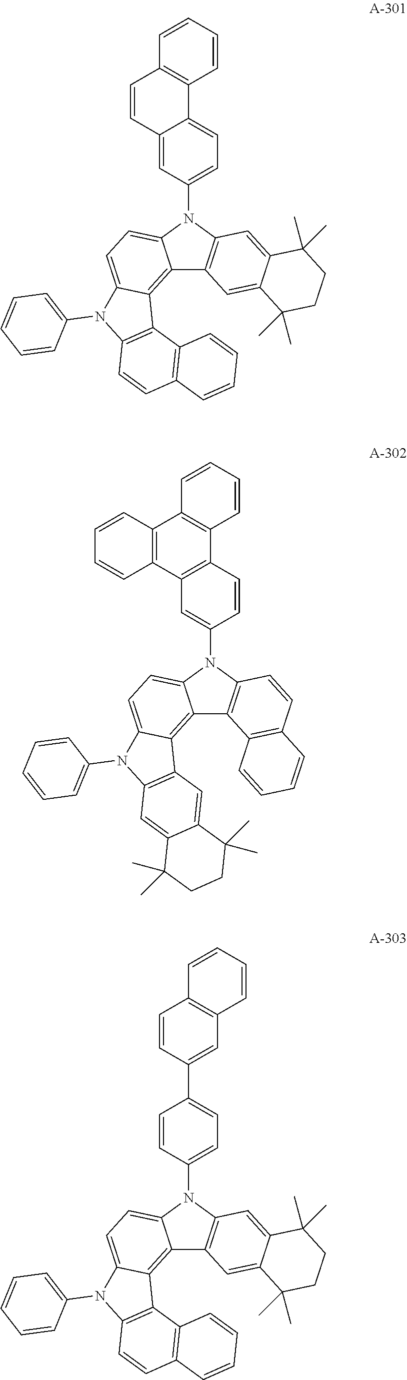 [SMILES: CC1(C)CCC(C)(C)c2cc3c(cc21)c1c2c4c5ccccc5ccc4n(-c4ccccc4)c2ccc1n3-c1ccc2c(ccc3ccccc32)c1 ; CC1(C)CCC(C)(C)c2cc3c(cc21)c1c2c4c5ccccc5ccc4n(-c4ccc5c6ccccc6c6ccccc6c5c4)c2ccc1n3-c1ccccc1 ; CC1(C)CCC(C)(C)c2cc3c(cc21)c1c2c4c5ccccc5ccc4n(-c4ccccc4)c2ccc1n3-c1ccc(-c2ccc3ccccc3c2)cc1]

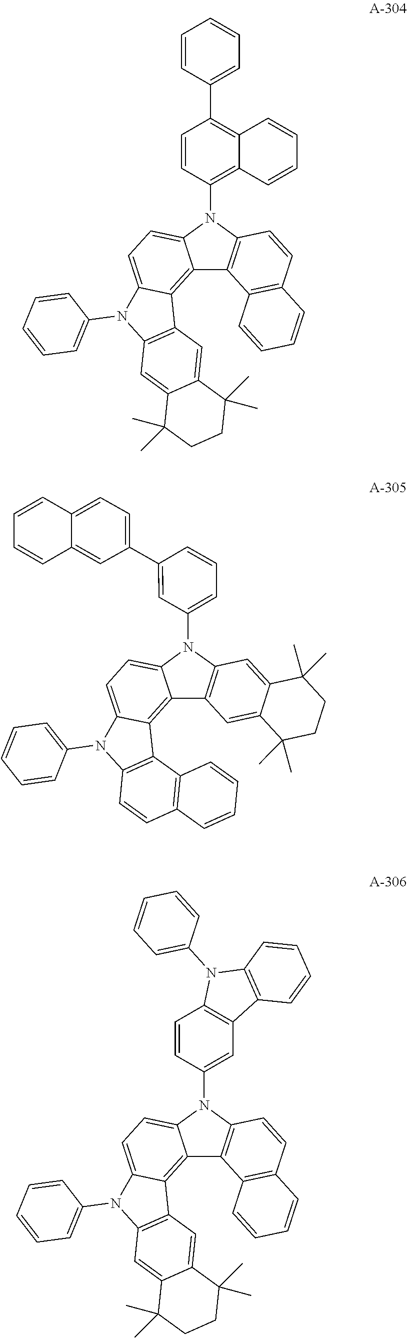 [SMILES: CC1(C)CCC(C)(C)c2cc3c(cc21)c1c2c4c5ccccc5ccc4n(-c4ccc(-c5ccccc5)c5ccccc45)c2ccc1n3-c1ccccc1 ; CC1(C)CCC(C)(C)c2cc3c(cc21)c1c2c4c5ccccc5ccc4n(-c4ccccc4)c2ccc1n3-c1cccc(-c2ccc3ccccc3c2)c1 ; CC1(C)CCC(C)(C)c2cc3c(cc21)c1c2c4c5ccccc5ccc4n(-c4ccc5c(c4)c4ccccc4n5-c4ccccc4)c2ccc1n3-c1ccccc1]

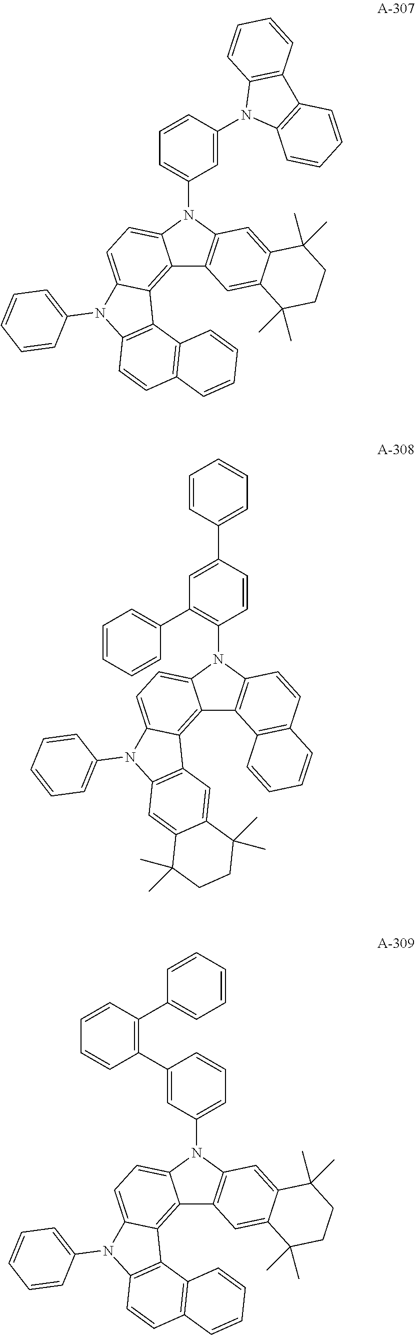 [SMILES: CC1(C)CCC(C)(C)c2cc3c(cc21)c1c2c4c5ccccc5ccc4n(-c4ccccc4)c2ccc1n3-c1cccc(-n2c3ccccc3c3ccccc32)c1 ; CC1(C)CCC(C)(C)c2cc3c(cc21)c1c2c4c5ccccc5ccc4n(-c4ccc(-c5ccccc5)cc4-c4ccccc4)c2ccc1n3-c1ccccc1 ; CC1(C)CCC(C)(C)c2cc3c(cc21)c1c2c4c5ccccc5ccc4n(-c4ccccc4)c2ccc1n3-c1cccc(-c2ccccc2-c2ccccc2)c1]

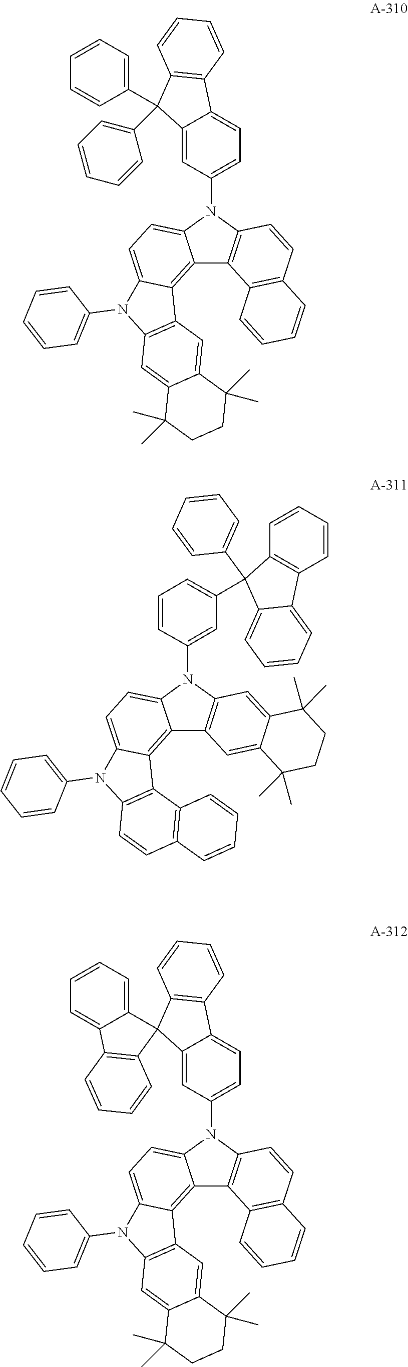 [SMILES: CC1(C)CCC(C)(C)c2cc3c(cc21)c1c2c4c5ccccc5ccc4n(-c4ccc5c(c4)C(c4ccccc4)(c4ccccc4)c4ccccc4-5)c2ccc1n3-c1ccccc1 ; CC1(C)CCC(C)(C)c2cc3c(cc21)c1c2c4c5ccccc5ccc4n(-c4ccccc4)c2ccc1n3-c1cccc(C2(c3ccccc3)c3ccccc3-c3ccccc32)c1 ; CC1(C)CCC(C)(C)c2cc3c(cc21)c1c2c4c5ccccc5ccc4n(-c4ccc5c(c4)C4(c6ccccc6-c6ccccc64)c4ccccc4-5)c2ccc1n3-c1ccccc1]

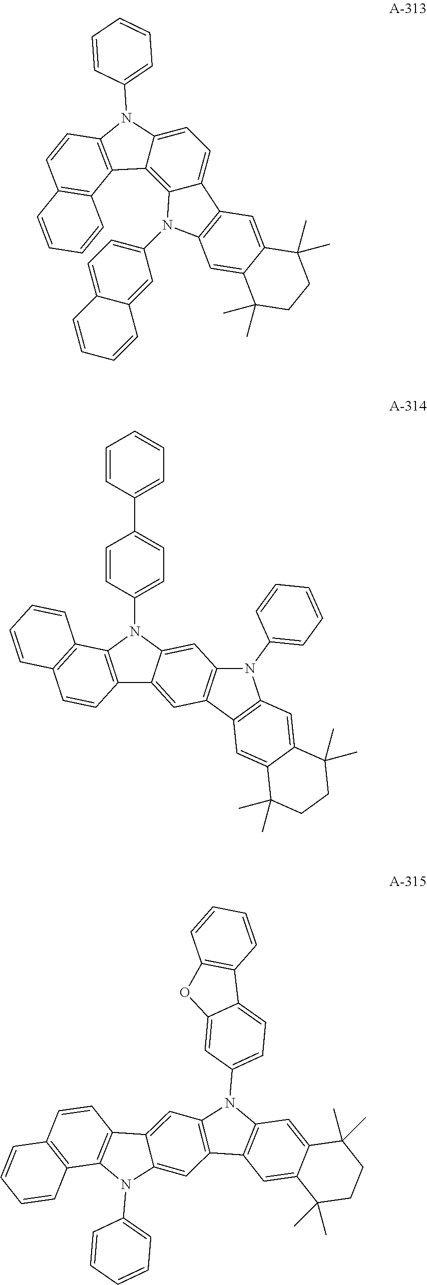 [SMILES: CC1(C)CCC(C)(C)c2cc3c(cc21)c1ccc2c(c4c5ccccc5ccc4n2-c2ccccc2)c1n3-c1ccc2ccccc2c1 ; CC1(C)CCC(C)(C)c2cc3c(cc21)c1cc2c4ccc5ccccc5c4n(-c4ccc(-c5ccccc5)cc4)c2cc1n3-c1ccccc1 ; CC1(C)CCC(C)(C)c2cc3c(cc21)c1cc2c(cc1n3-c1ccc3c(c1)oc1ccccc13)c1ccc3ccccc3c1n2-c1ccccc1]

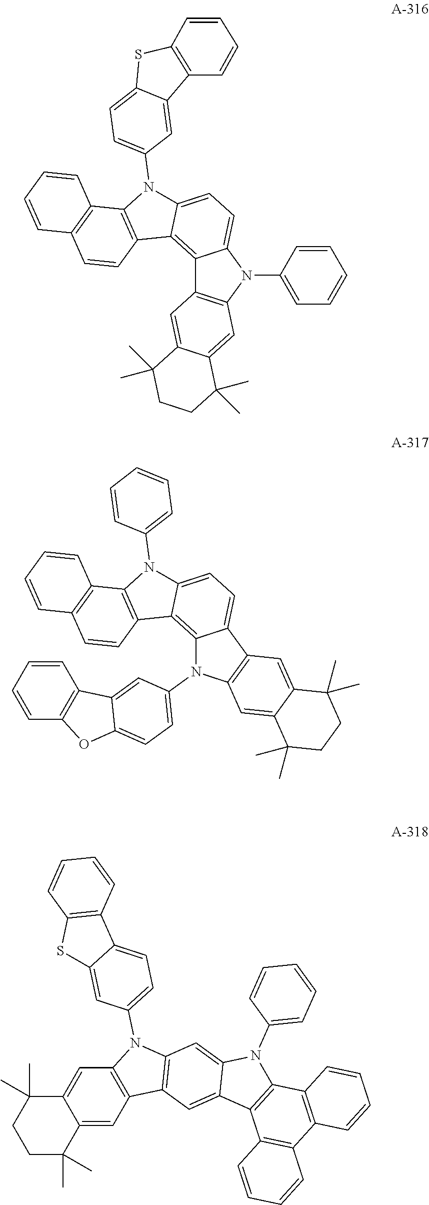

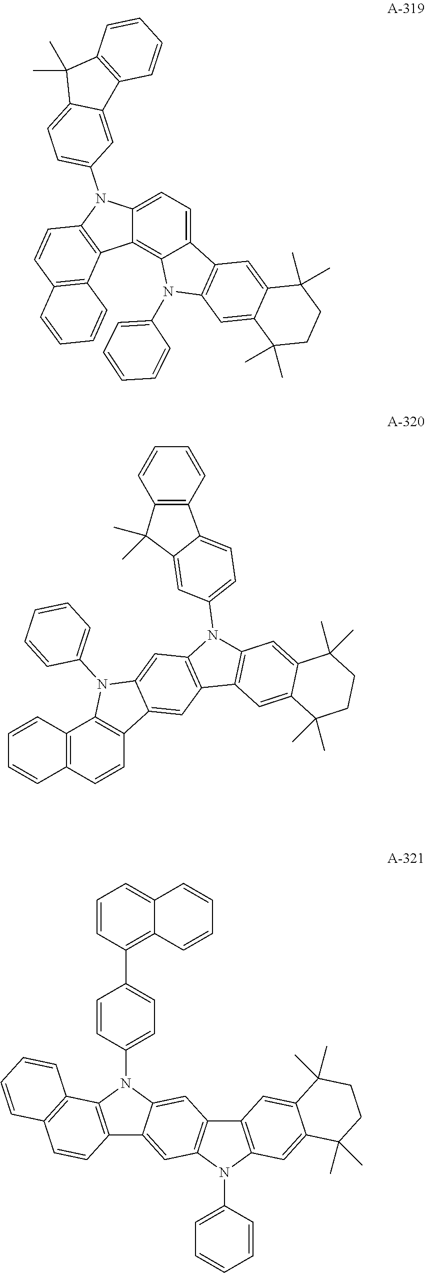

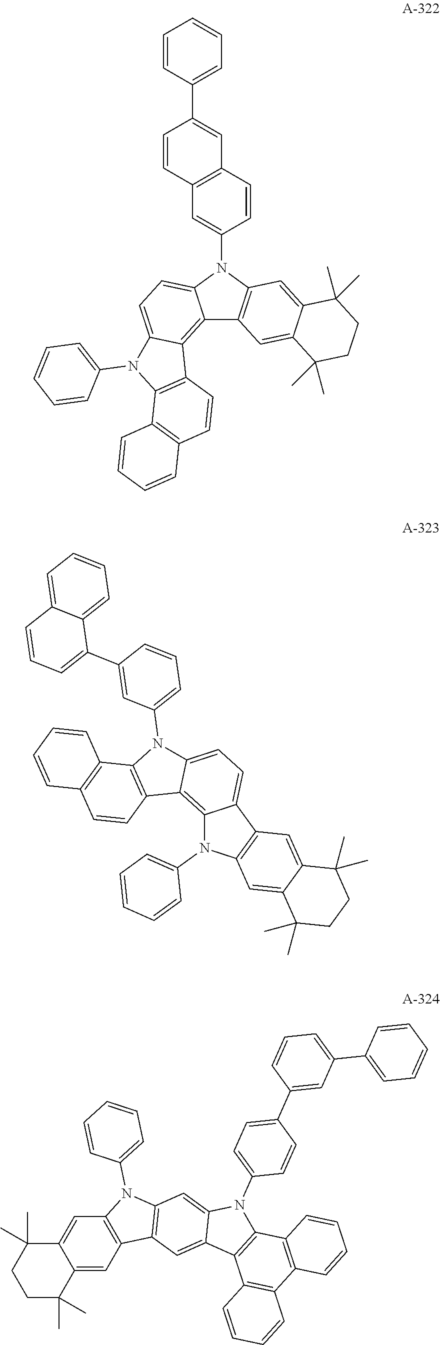

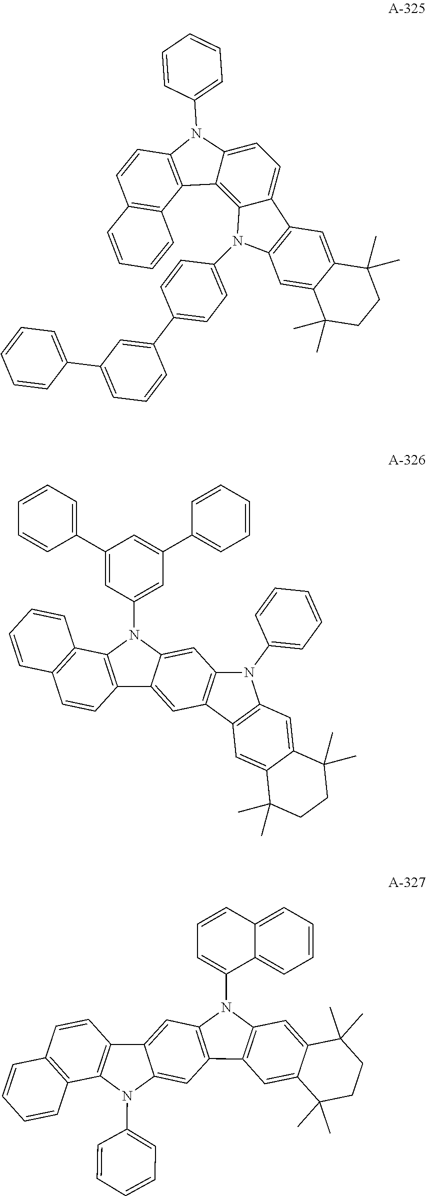 [SMILES: CC1(C)CCC(C)(C)c2cc3c(cc21)c1ccc2c(c4c5ccccc5ccc4n2-c2ccccc2)c1n3-c1ccc(-c2cccc(-c3ccccc3)c2)cc1 ; CC1(C)CCC(C)(C)c2cc3c(cc21)c1cc2c4ccc5ccccc5c4n(-c4cc(-c5ccccc5)cc(-c5ccccc5)c4)c2cc1n3-c1ccccc1 ; CC1(C)CCC(C)(C)c2cc3c(cc21)c1cc2c(cc1n3-c1cccc3ccccc13)c1ccc3ccccc3c1n2-c1ccccc1]

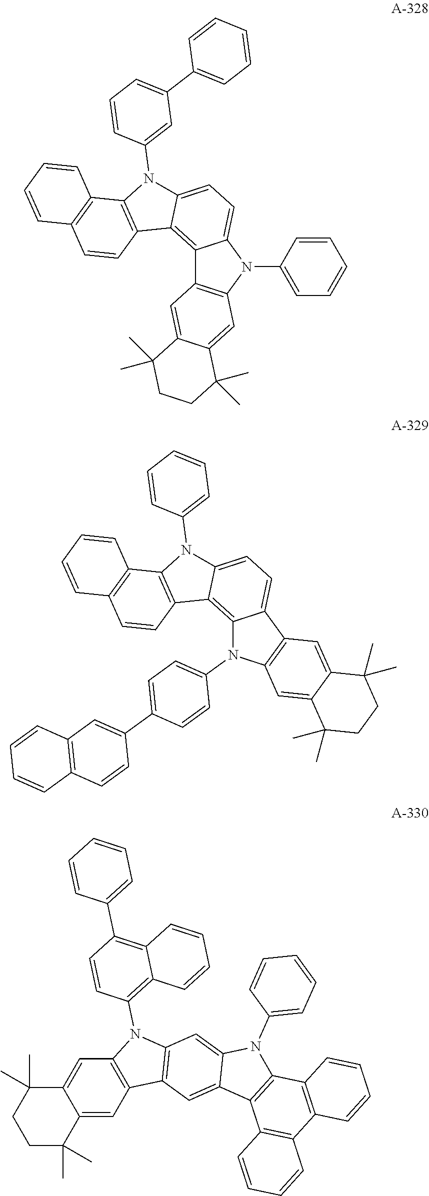

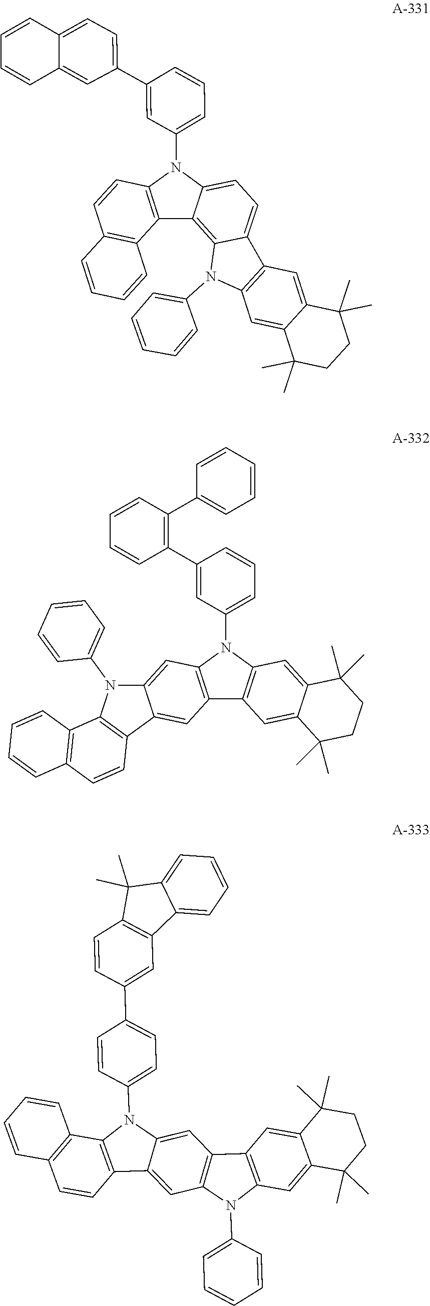 [SMILES: CC1(C)CCC(C)(C)c2cc3c(cc21)c1ccc2c(c4c5ccccc5ccc4n2-c2cccc(-c4ccc5ccccc5c4)c2)c1n3-c1ccccc1 ; CC1(C)CCC(C)(C)c2cc3c(cc21)c1cc2c4ccc5ccccc5c4n(-c4ccccc4)c2cc1n3-c1cccc(-c2ccccc2-c2ccccc2)c1 ; CC1(C)CCC(C)(C)c2cc3c(cc21)c1cc2c(cc1n3-c1ccccc1)c1ccc3ccccc3c1n2-c1ccc(-c2ccc3c(c2)-c2ccccc2C3(C)C)cc1]

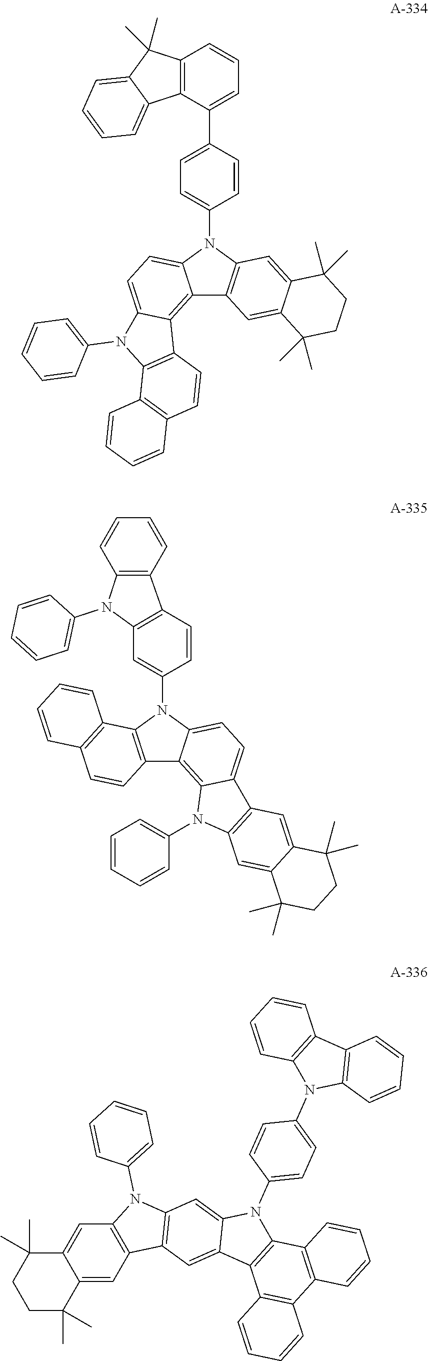

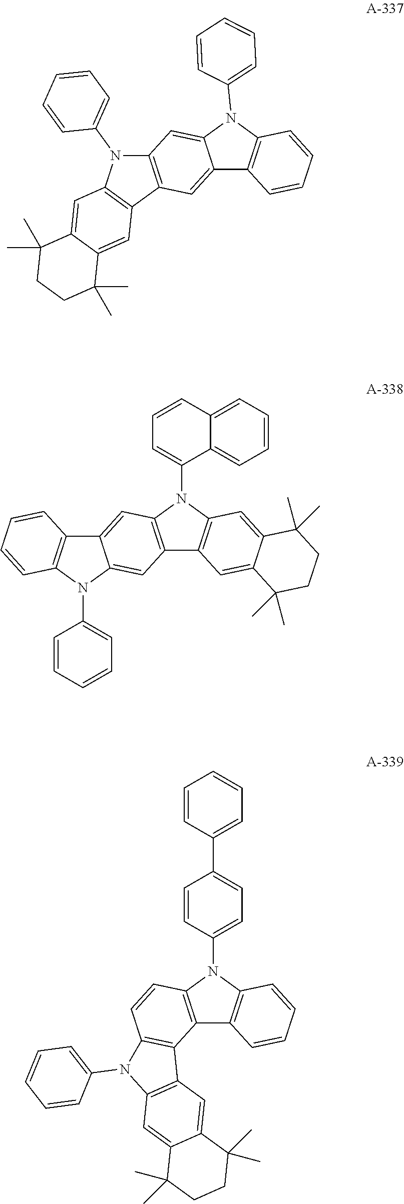 [SMILES: CC1(C)CCC(C)(C)c2cc3c(cc21)c1cc2c4ccccc4n(-c4ccccc4)c2cc1n3-c1ccccc1 ; CC1(C)CCC(C)(C)c2cc3c(cc21)c1cc2c(cc1n3-c1cccc3ccccc13)c1ccccc1n2-c1ccccc1 ; CC1(C)CCC(C)(C)c2cc3c(cc21)c1c2c4ccccc4n(-c4ccc(-c5ccccc5)cc4)c2ccc1n3-c1ccccc1]

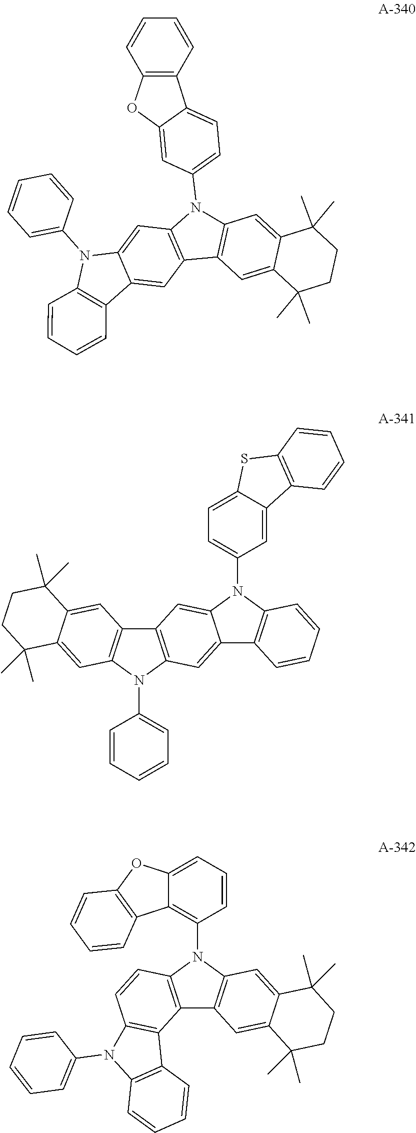 [SMILES: CC1(C)CCC(C)(C)c2cc3c(cc21)c1cc2c4ccccc4n(-c4ccccc4)c2cc1n3-c1ccc2c(c1)oc1ccccc12 ; CC1(C)CCC(C)(C)c2cc3c(cc21)c1cc2c(cc1n3-c1ccccc1)c1ccccc1n2-c1ccc2sc3ccccc3c2c1 ; CC1(C)CCC(C)(C)c2cc3c(cc21)c1c2c4ccccc4n(-c4ccccc4)c2ccc1n3-c1cccc2oc3ccccc3c12]

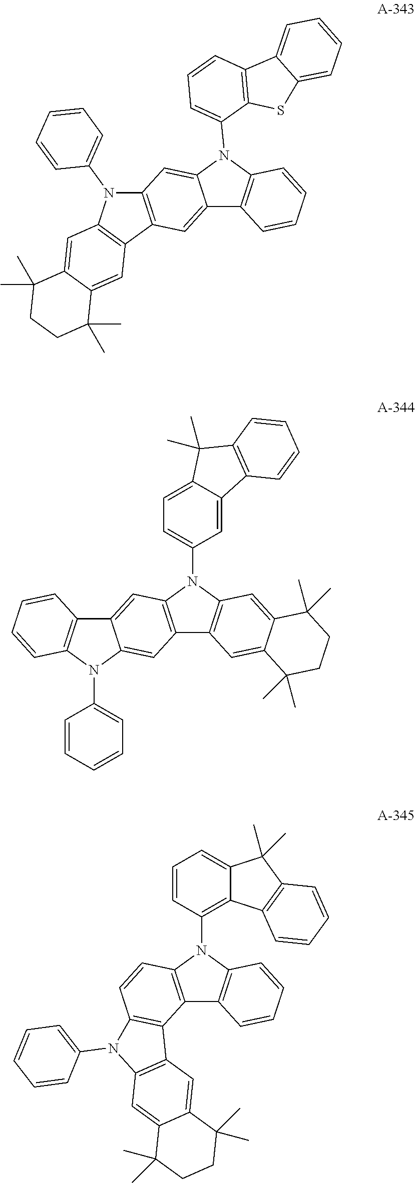 [SMILES: CC1(C)CCC(C)(C)c2cc3c(cc21)c1cc2c4ccccc4n(-c4cccc5c4sc4ccccc45)c2cc1n3-c1ccccc1 ; CC1(C)CCC(C)(C)c2cc3c(cc21)c1cc2c(cc1n3-c1ccc3c(c1)-c1ccccc1C3(C)C)c1ccccc1n2-c1ccccc1 ; CC1(C)CCC(C)(C)c2cc3c(cc21)c1c2c4ccccc4n(-c4cccc5c4-c4ccccc4C5(C)C)c2ccc1n3-c1ccccc1]

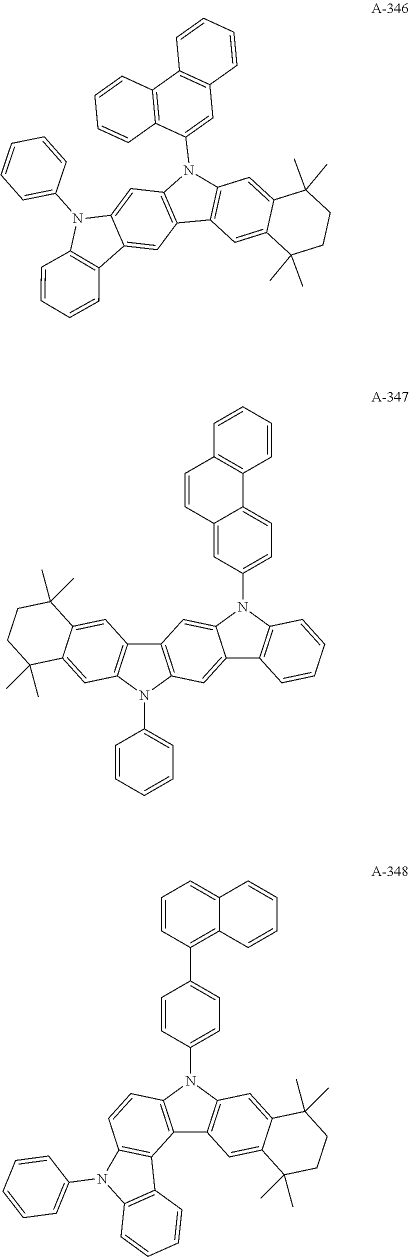 [SMILES: CC1(C)CCC(C)(C)c2cc3c(cc21)c1cc2c4ccccc4n(-c4ccccc4)c2cc1n3-c1cc2ccccc2c2ccccc12 ; CC1(C)CCC(C)(C)c2cc3c(cc21)c1cc2c(cc1n3-c1ccccc1)c1ccccc1n2-c1ccc2c(ccc3ccccc32)c1 ; CC1(C)CCC(C)(C)c2cc3c(cc21)c1c2c4ccccc4n(-c4ccccc4)c2ccc1n3-c1ccc(-c2cccc3ccccc23)cc1]

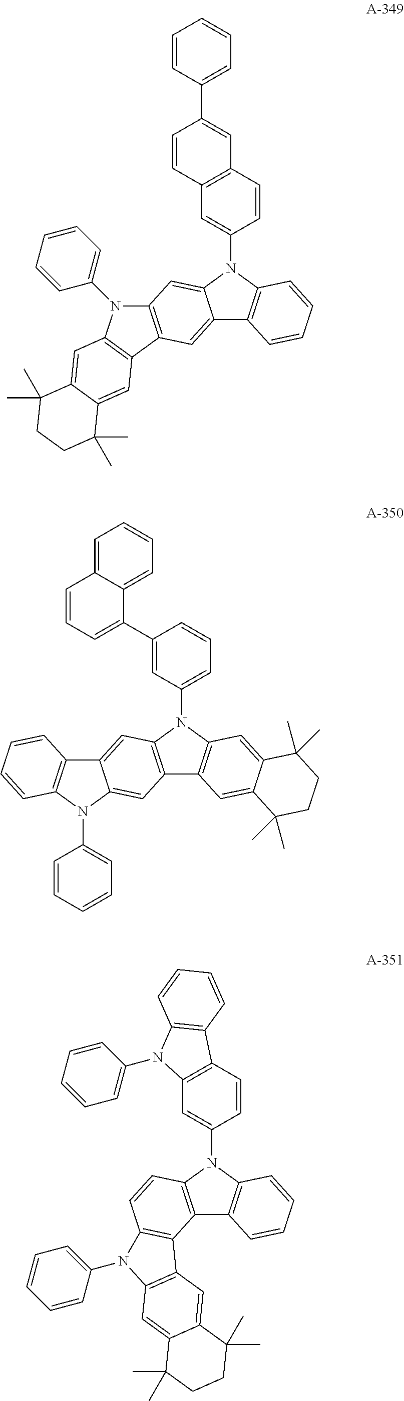 [SMILES: CC1(C)CCC(C)(C)c2cc3c(cc21)c1cc2c4ccccc4n(-c4ccc5cc(-c6ccccc6)ccc5c4)c2cc1n3-c1ccccc1 ; CC1(C)CCC(C)(C)c2cc3c(cc21)c1cc2c(cc1n3-c1cccc(-c3cccc4ccccc34)c1)c1ccccc1n2-c1ccccc1 ; CC1(C)CCC(C)(C)c2cc3c(cc21)c1c2c4ccccc4n(-c4ccc5c6ccccc6n(-c6ccccc6)c5c4)c2ccc1n3-c1ccccc1]

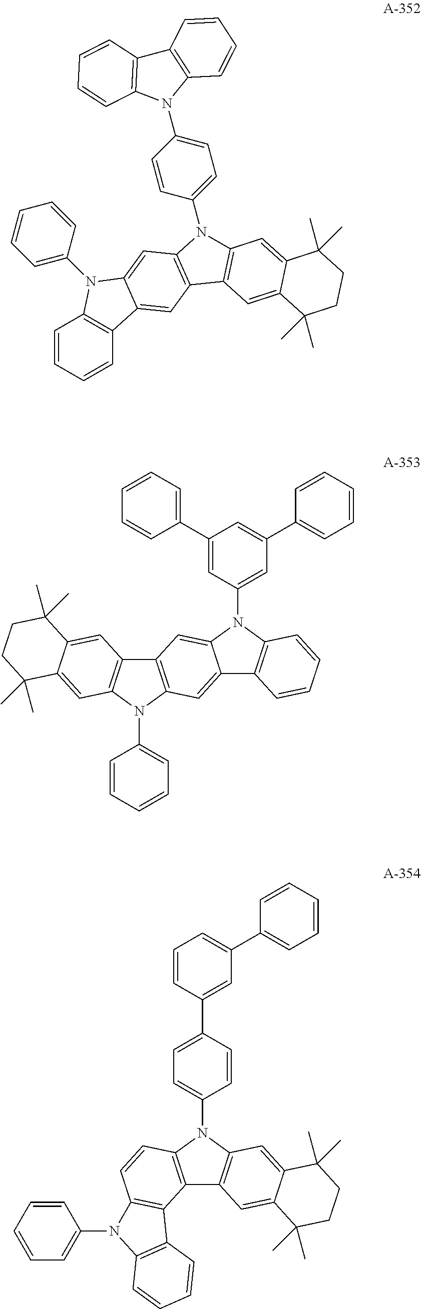 [SMILES: CC1(C)CCC(C)(C)c2cc3c(cc21)c1cc2c4ccccc4n(-c4ccccc4)c2cc1n3-c1ccc(-n2c3ccccc3c3ccccc32)cc1 ; CC1(C)CCC(C)(C)c2cc3c(cc21)c1cc2c(cc1n3-c1ccccc1)c1ccccc1n2-c1cc(-c2ccccc2)cc(-c2ccccc2)c1 ; CC1(C)CCC(C)(C)c2cc3c(cc21)c1c2c4ccccc4n(-c4ccccc4)c2ccc1n3-c1ccc(-c2cccc(-c3ccccc3)c2)cc1]

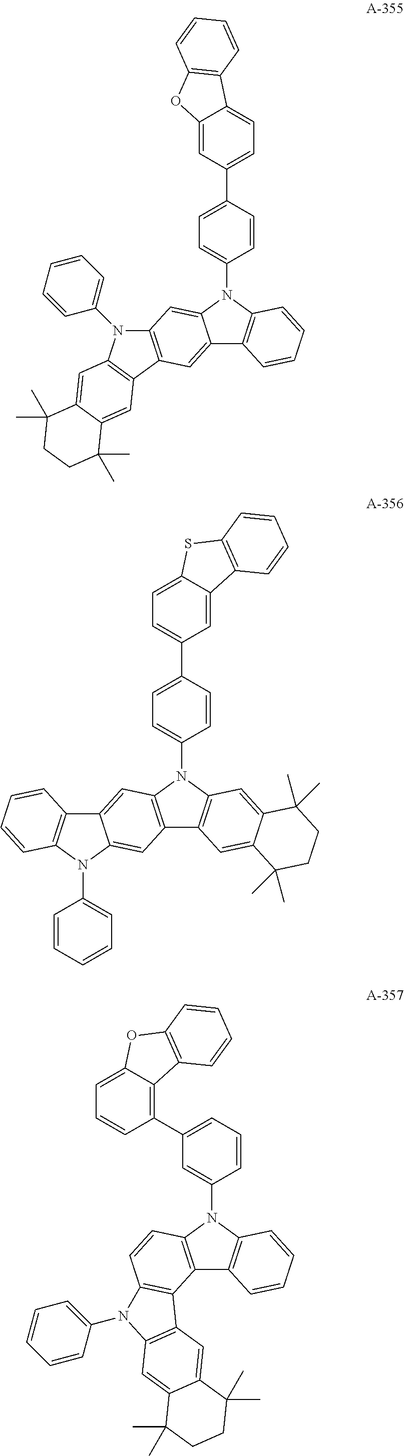 [SMILES: CC1(C)CCC(C)(C)c2cc3c(cc21)c1cc2c4ccccc4n(-c4ccc(-c5ccc6c(c5)oc5ccccc56)cc4)c2cc1n3-c1ccccc1 ; CC1(C)CCC(C)(C)c2cc3c(cc21)c1cc2c(cc1n3-c1ccc(-c3ccc4sc5ccccc5c4c3)cc1)c1ccccc1n2-c1ccccc1 ; CC1(C)CCC(C)(C)c2cc3c(cc21)c1c2c4ccccc4n(-c4cccc(-c5cccc6oc7ccccc7c56)c4)c2ccc1n3-c1ccccc1]

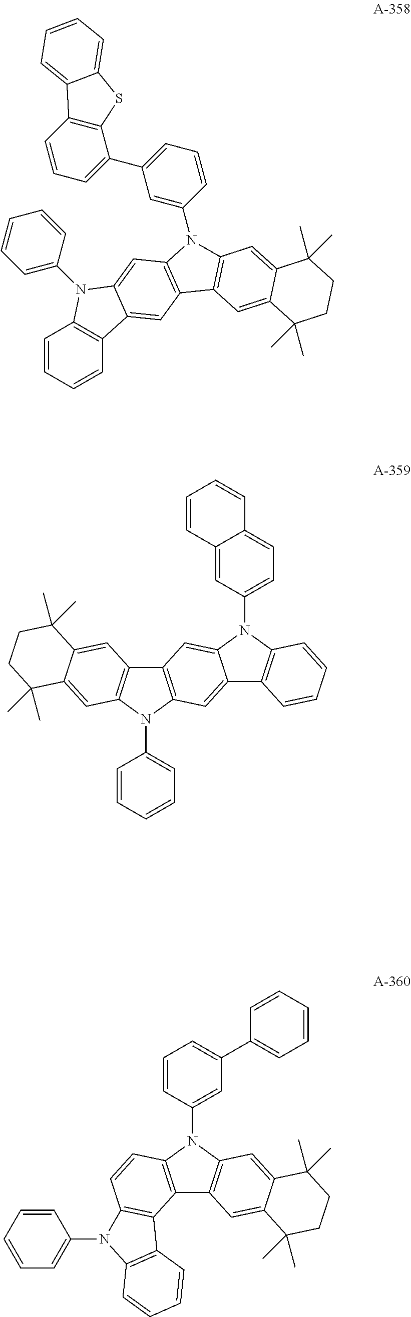 [SMILES: CC1(C)CCC(C)(C)c2cc3c(cc21)c1cc2c4ccccc4n(-c4ccccc4)c2cc1n3-c1cccc(-c2cccc3c2sc2ccccc23)c1 ; CC1(C)CCC(C)(C)c2cc3c(cc21)c1cc2c(cc1n3-c1ccccc1)c1ccccc1n2-c1ccc2ccccc2c1 ; CC1(C)CCC(C)(C)c2cc3c(cc21)c1c2c4ccccc4n(-c4ccccc4)c2ccc1n3-c1cccc(-c2ccccc2)c1]

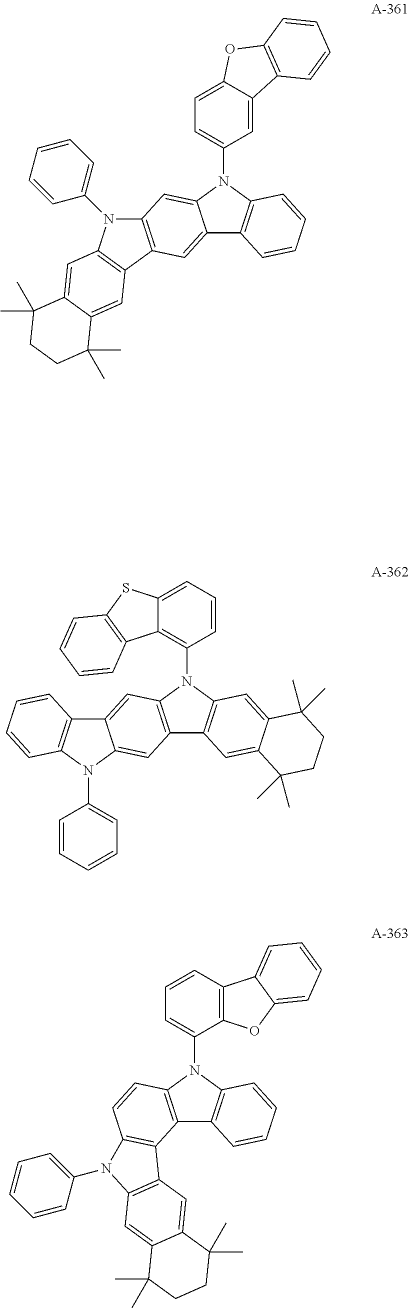 [SMILES: CC1(C)CCC(C)(C)c2cc3c(cc21)c1cc2c4ccccc4n(-c4ccc5oc6ccccc6c5c4)c2cc1n3-c1ccccc1 ; CC1(C)CCC(C)(C)c2cc3c(cc21)c1cc2c(cc1n3-c1cccc3sc4ccccc4c13)c1ccccc1n2-c1ccccc1 ; CC1(C)CCC(C)(C)c2cc3c(cc21)c1c2c4ccccc4n(-c4cccc5c4oc4ccccc45)c2ccc1n3-c1ccccc1]

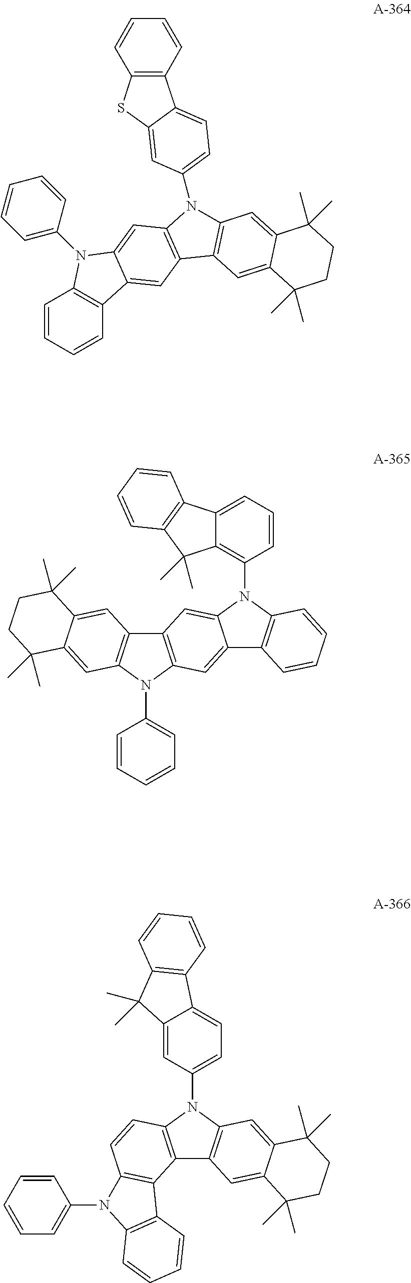 [SMILES: CC1(C)CCC(C)(C)c2cc3c(cc21)c1cc2c4ccccc4n(-c4ccccc4)c2cc1n3-c1ccc2c(c1)sc1ccccc12 ; CC1(C)CCC(C)(C)c2cc3c(cc21)c1cc2c(cc1n3-c1ccccc1)c1ccccc1n2-c1cccc2c1C(C)(C)c1ccccc1-2 ; CC1(C)CCC(C)(C)c2cc3c(cc21)c1c2c4ccccc4n(-c4ccccc4)c2ccc1n3-c1ccc2c(c1)C(C)(C)c1ccccc1-2]

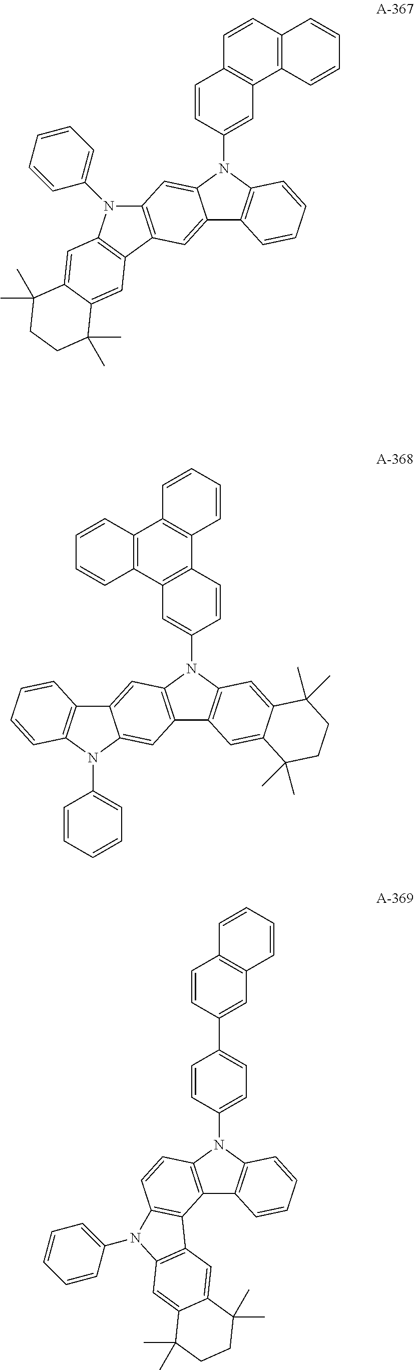 [SMILES: CC1(C)CCC(C)(C)c2cc3c(cc21)c1cc2c4ccccc4n(-c4ccc5ccc6ccccc6c5c4)c2cc1n3-c1ccccc1 ; CC1(C)CCC(C)(C)c2cc3c(cc21)c1cc2c(cc1n3-c1ccc3c4ccccc4c4ccccc4c3c1)c1ccccc1n2-c1ccccc1 ; CC1(C)CCC(C)(C)c2cc3c(cc21)c1c2c4ccccc4n(-c4ccc(-c5ccc6ccccc6c5)cc4)c2ccc1n3-c1ccccc1]

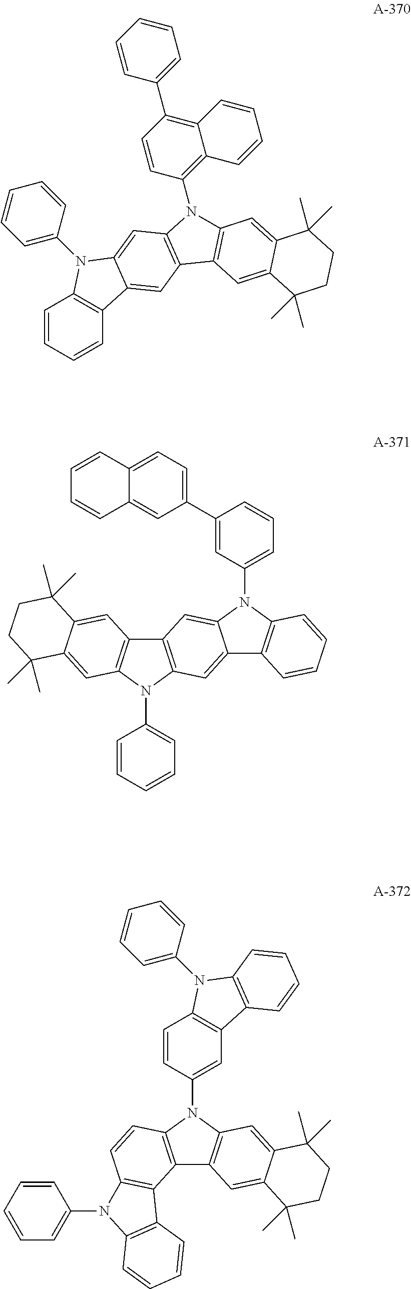 [SMILES: CC1(C)CCC(C)(C)c2cc3c(cc21)c1cc2c4ccccc4n(-c4ccccc4)c2cc1n3-c1ccc(-c2ccccc2)c2ccccc12 ; CC1(C)CCC(C)(C)c2cc3c(cc21)c1cc2c(cc1n3-c1ccccc1)c1ccccc1n2-c1cccc(-c2ccc3ccccc3c2)c1 ; CC1(C)CCC(C)(C)c2cc3c(cc21)c1c2c4ccccc4n(-c4ccccc4)c2ccc1n3-c1ccc2c(c1)c1ccccc1n2-c1ccccc1]

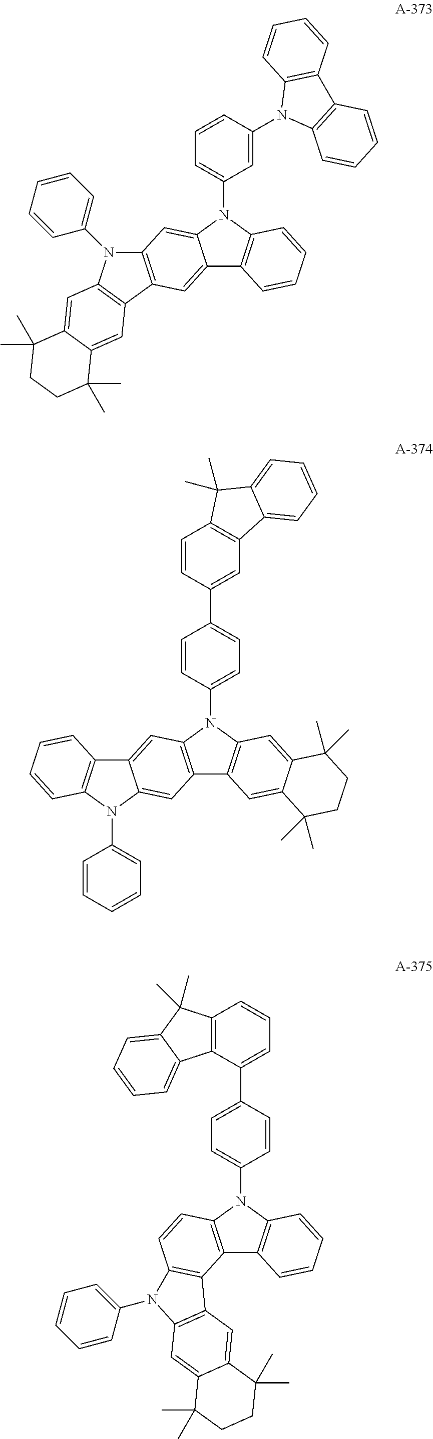 [SMILES: CC1(C)CCC(C)(C)c2cc3c(cc21)c1cc2c4ccccc4n(-c4cccc(-n5c6ccccc6c6ccccc65)c4)c2cc1n3-c1ccccc1 ; CC1(C)CCC(C)(C)c2cc3c(cc21)c1cc2c(cc1n3-c1ccc(-c3ccc4c(c3)-c3ccccc3C4(C)C)cc1)c1ccccc1n2-c1ccccc1 ; CC1(C)CCC(C)(C)c2cc3c(cc21)c1c2c4ccccc4n(-c4ccc(-c5cccc6c5-c5ccccc5C6(C)C)cc4)c2ccc1n3-c1ccccc1]

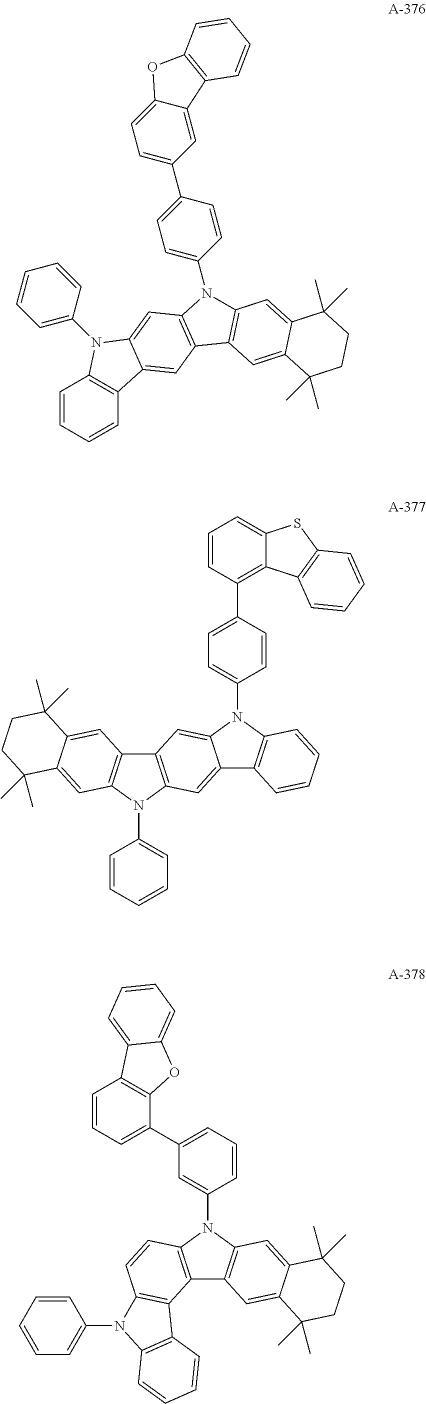 [SMILES: CC1(C)CCC(C)(C)c2cc3c(cc21)c1cc2c4ccccc4n(-c4ccccc4)c2cc1n3-c1ccc(-c2ccc3oc4ccccc4c3c2)cc1 ; CC1(C)CCC(C)(C)c2cc3c(cc21)c1cc2c(cc1n3-c1ccccc1)c1ccccc1n2-c1ccc(-c2cccc3sc4ccccc4c23)cc1 ; CC1(C)CCC(C)(C)c2cc3c(cc21)c1c2c4ccccc4n(-c4ccccc4)c2ccc1n3-c1cccc(-c2cccc3c2oc2ccccc23)c1]

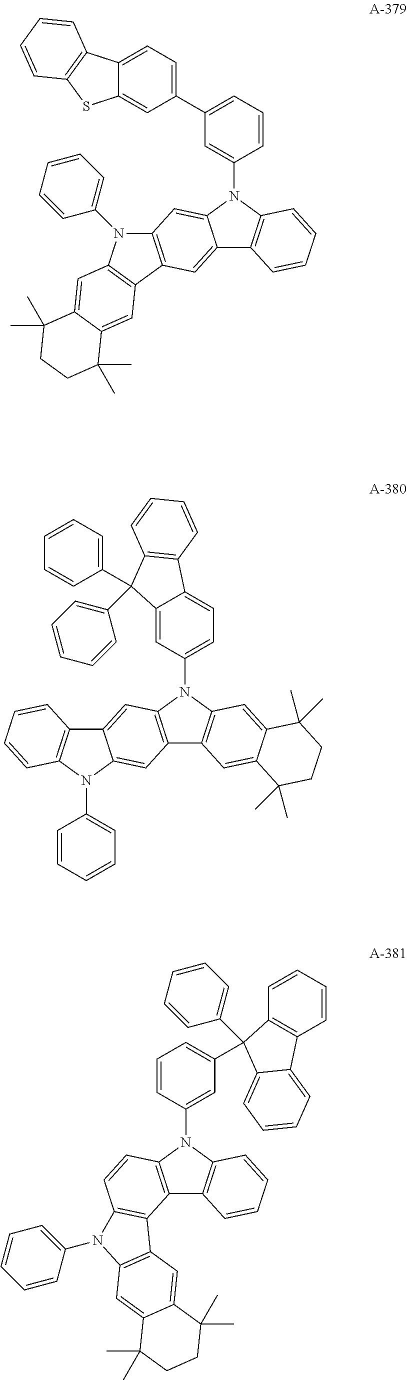 [SMILES: CC1(C)CCC(C)(C)c2cc3c(cc21)c1cc2c4ccccc4n(-c4cccc(-c5ccc6c(c5)sc5ccccc56)c4)c2cc1n3-c1ccccc1 ; CC1(C)CCC(C)(C)c2cc3c(cc21)c1cc2c(cc1n3-c1ccc3c(c1)C(c1ccccc1)(c1ccccc1)c1ccccc1-3)c1ccccc1n2-c1ccccc1 ; CC1(C)CCC(C)(C)c2cc3c(cc21)c1c2c4ccccc4n(-c4cccc(C5(c6ccccc6)c6ccccc6-c6ccccc65)c4)c2ccc1n3-c1ccccc1]

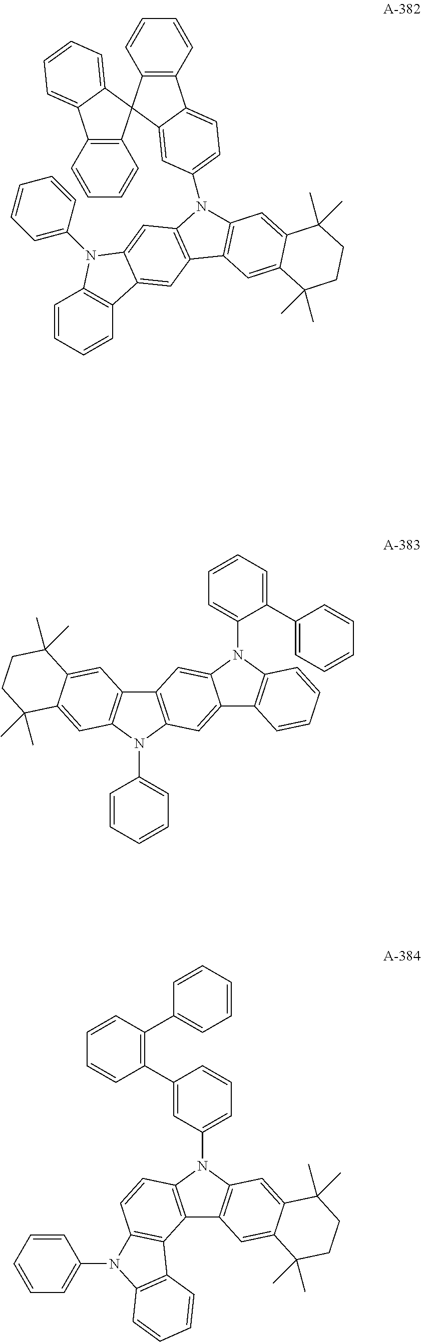 [SMILES: CC1(C)CCC(C)(C)c2cc3c(cc21)c1cc2c4ccccc4n(-c4ccccc4)c2cc1n3-c1ccc2c(c1)C1(c3ccccc3-c3ccccc31)c1ccccc1-2 ; CC1(C)CCC(C)(C)c2cc3c(cc21)c1cc2c(cc1n3-c1ccccc1)c1ccccc1n2-c1ccccc1-c1ccccc1 ; CC1(C)CCC(C)(C)c2cc3c(cc21)c1c2c4ccccc4n(-c4ccccc4)c2ccc1n3-c1cccc(-c2ccccc2-c2ccccc2)c1]

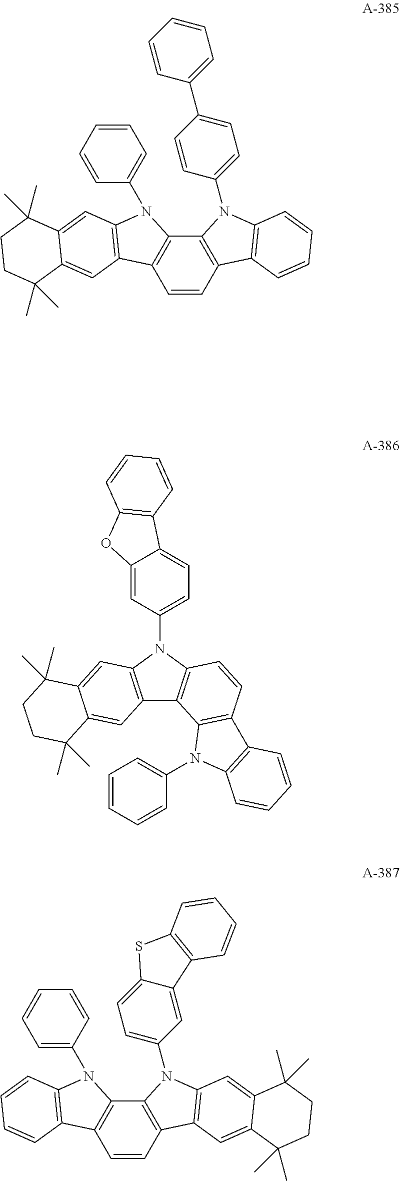 [SMILES: CC1(C)CCC(C)(C)c2cc3c(cc21)c1ccc2c4ccccc4n(-c4ccc(-c5ccccc5)cc4)c2c1n3-c1ccccc1 ; CC1(C)CCC(C)(C)c2cc3c(cc21)c1c(ccc2c4ccccc4n(-c4ccccc4)c21)n3-c1ccc2c(c1)oc1ccccc12 ; CC1(C)CCC(C)(C)c2cc3c(cc21)c1ccc2c4ccccc4n(-c4ccccc4)c2c1n3-c1ccc2sc3ccccc3c2c1]

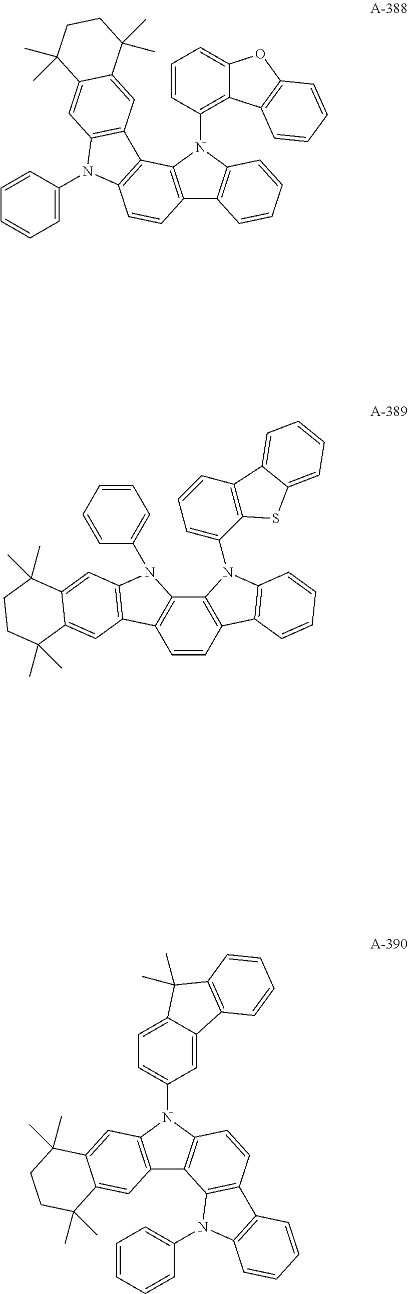 [SMILES: CC1(C)CCC(C)(C)c2cc3c(cc21)c1c(ccc2c4ccccc4n(-c4cccc5oc6ccccc6c45)c21)n3-c1ccccc1 ; CC1(C)CCC(C)(C)c2cc3c(cc21)c1ccc2c4ccccc4n(-c4cccc5c4sc4ccccc45)c2c1n3-c1ccccc1 ; CC1(C)CCC(C)(C)c2cc3c(cc21)c1c(ccc2c4ccccc4n(-c4ccccc4)c21)n3-c1ccc2c(c1)-c1ccccc1C2(C)C]

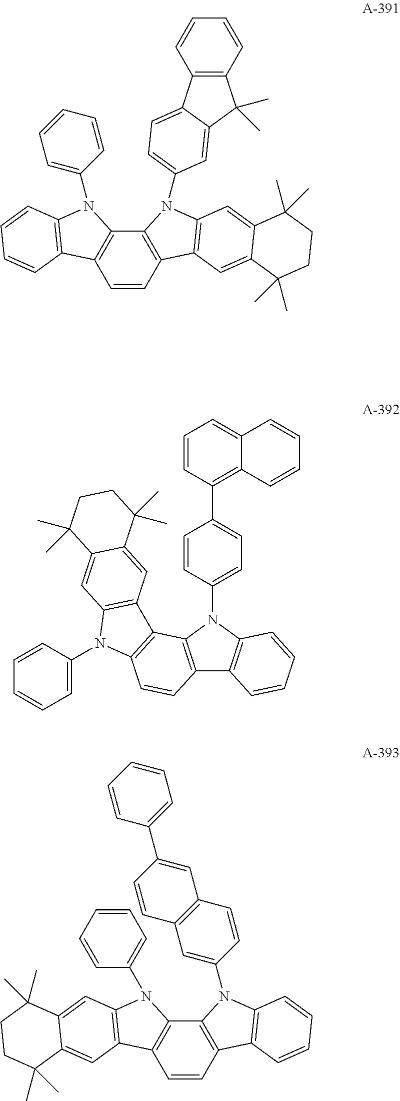 [SMILES: CC1(C)CCC(C)(C)c2cc3c(cc21)c1ccc2c4ccccc4n(-c4ccccc4)c2c1n3-c1ccc2c(c1)C(C)(C)c1ccccc1-2 ; CC1(C)CCC(C)(C)c2cc3c(cc21)c1c(ccc2c4ccccc4n(-c4ccc(-c5cccc6ccccc56)cc4)c21)n3-c1ccccc1 ; CC1(C)CCC(C)(C)c2cc3c(cc21)c1ccc2c4ccccc4n(-c4ccc5cc(-c6ccccc6)ccc5c4)c2c1n3-c1ccccc1]

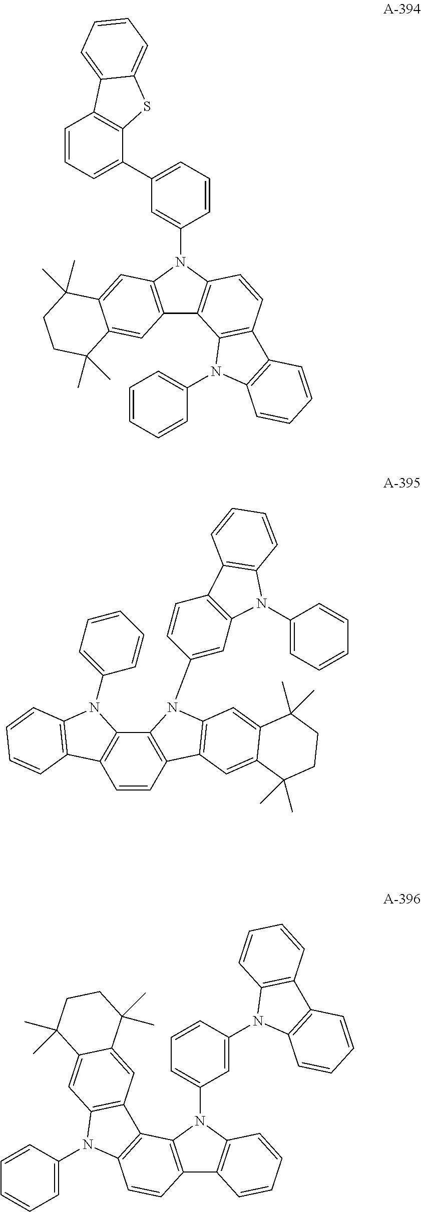 [SMILES: CC1(C)CCC(C)(C)c2cc3c(cc21)c1c(ccc2c4ccccc4n(-c4ccccc4)c21)n3-c1cccc(-c2cccc3c2sc2ccccc23)c1 ; CC1(C)CCC(C)(C)c2cc3c(cc21)c1ccc2c4ccccc4n(-c4ccccc4)c2c1n3-c1ccc2c3ccccc3n(-c3ccccc3)c2c1 ; CC1(C)CCC(C)(C)c2cc3c(cc21)c1c(ccc2c4ccccc4n(-c4cccc(-n5c6ccccc6c6ccccc65)c4)c21)n3-c1ccccc1]

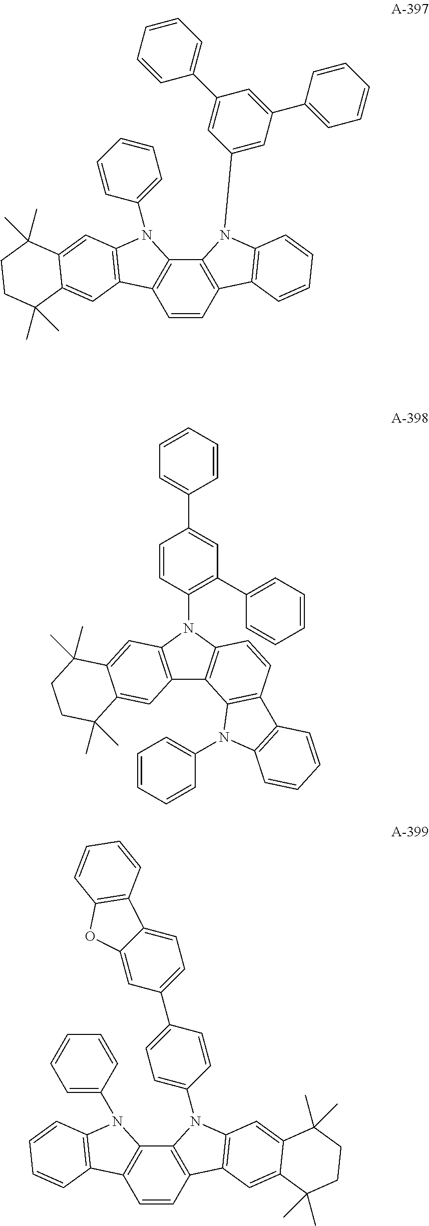 [SMILES: CC1(C)CCC(C)(C)c2cc3c(cc21)c1ccc2c4ccccc4n(-c4cc(-c5ccccc5)cc(-c5ccccc5)c4)c2c1n3-c1ccccc1 ; CC1(C)CCC(C)(C)c2cc3c(cc21)c1c(ccc2c4ccccc4n(-c4ccccc4)c21)n3-c1ccc(-c2ccccc2)cc1-c1ccccc1 ; CC1(C)CCC(C)(C)c2cc3c(cc21)c1ccc2c4ccccc4n(-c4ccccc4)c2c1n3-c1ccc(-c2ccc3c(c2)oc2ccccc23)cc1]

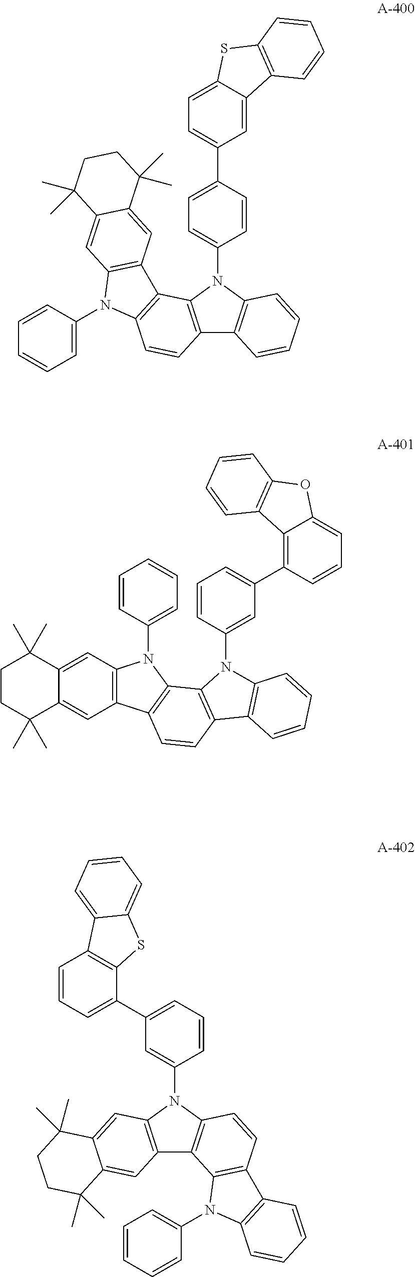 [SMILES: CC1(C)CCC(C)(C)c2cc3c(cc21)c1c(ccc2c4ccccc4n(-c4ccc(-c5ccc6sc7ccccc7c6c5)cc4)c21)n3-c1ccccc1 ; CC1(C)CCC(C)(C)c2cc3c(cc21)c1ccc2c4ccccc4n(-c4cccc(-c5cccc6oc7ccccc7c56)c4)c2c1n3-c1ccccc1 ; CC1(C)CCC(C)(C)c2cc3c(cc21)c1c(ccc2c4ccccc4n(-c4ccccc4)c21)n3-c1cccc(-c2cccc3c2sc2ccccc23)c1]

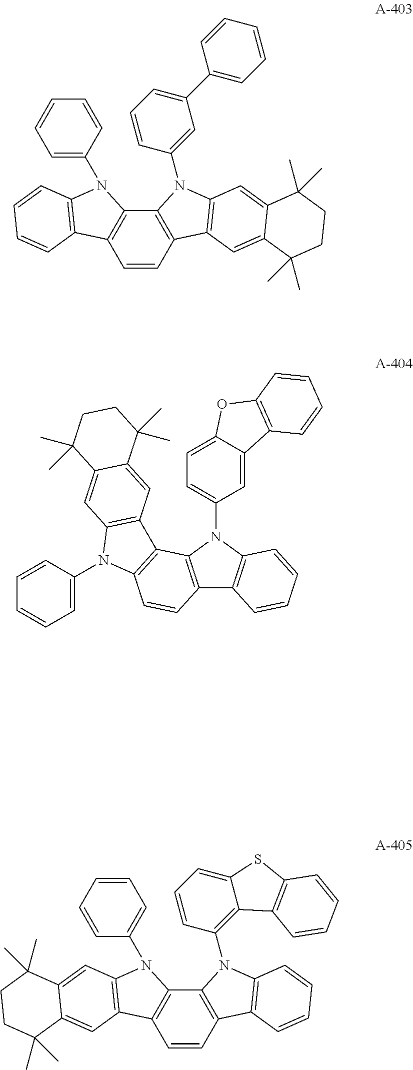 [SMILES: CC1(C)CCC(C)(C)c2cc3c(cc21)c1ccc2c4ccccc4n(-c4ccccc4)c2c1n3-c1cccc(-c2ccccc2)c1 ; CC1(C)CCC(C)(C)c2cc3c(cc21)c1c(ccc2c4ccccc4n(-c4ccc5oc6ccccc6c5c4)c21)n3-c1ccccc1 ; CC1(C)CCC(C)(C)c2cc3c(cc21)c1ccc2c4ccccc4n(-c4cccc5sc6ccccc6c45)c2c1n3-c1ccccc1]

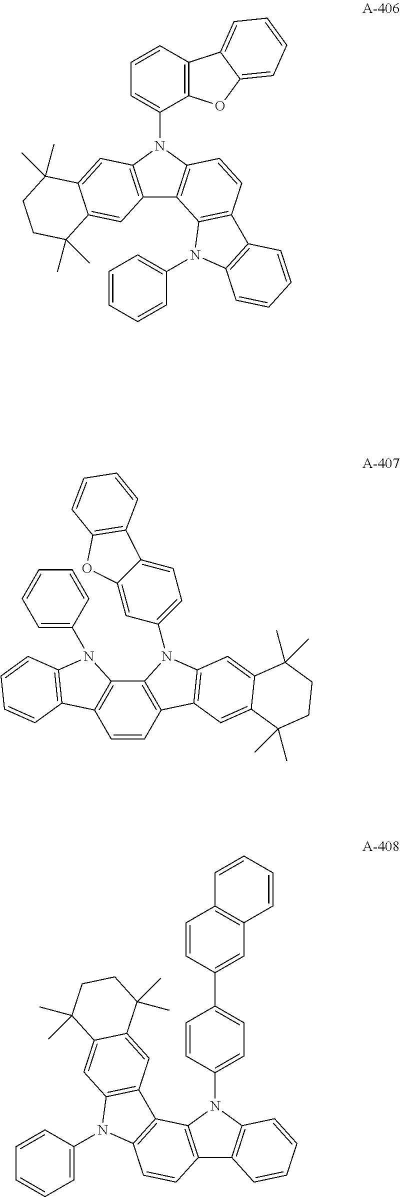 [SMILES: CC1(C)CCC(C)(C)c2cc3c(cc21)c1c(ccc2c4ccccc4n(-c4ccccc4)c21)n3-c1cccc2c1oc1ccccc12 ; CC1(C)CCC(C)(C)c2cc3c(cc21)c1ccc2c4ccccc4n(-c4ccccc4)c2c1n3-c1ccc2c(c1)oc1ccccc12 ; CC1(C)CCC(C)(C)c2cc3c(cc21)c1c(ccc2c4ccccc4n(-c4ccc(-c5ccc6ccccc6c5)cc4)c21)n3-c1ccccc1]

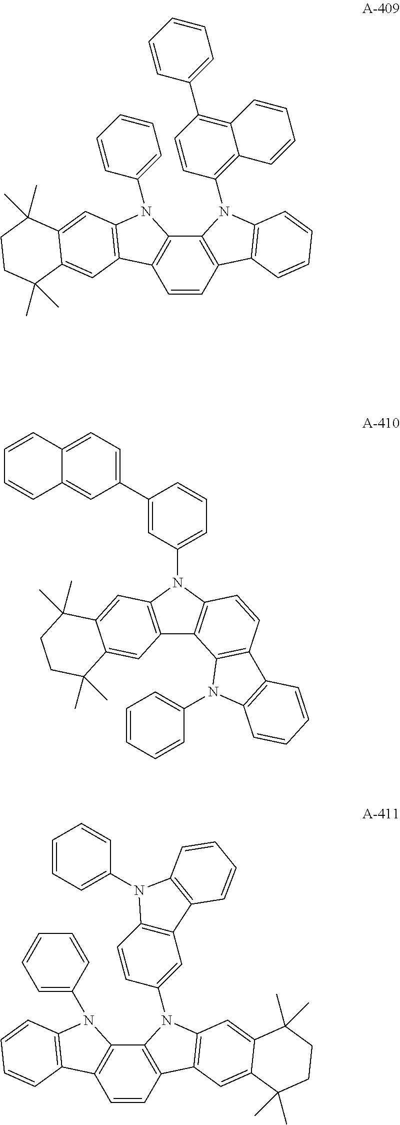 [SMILES: CC1(C)CCC(C)(C)c2cc3c(cc21)c1ccc2c4ccccc4n(-c4ccc(-c5ccccc5)c5ccccc45)c2c1n3-c1ccccc1 ; CC1(C)CCC(C)(C)c2cc3c(cc21)c1c(ccc2c4ccccc4n(-c4ccccc4)c21)n3-c1cccc(-c2ccc3ccccc3c2)c1 ; CC1(C)CCC(C)(C)c2cc3c(cc21)c1ccc2c4ccccc4n(-c4ccccc4)c2c1n3-c1ccc2c(c1)c1ccccc1n2-c1ccccc1]

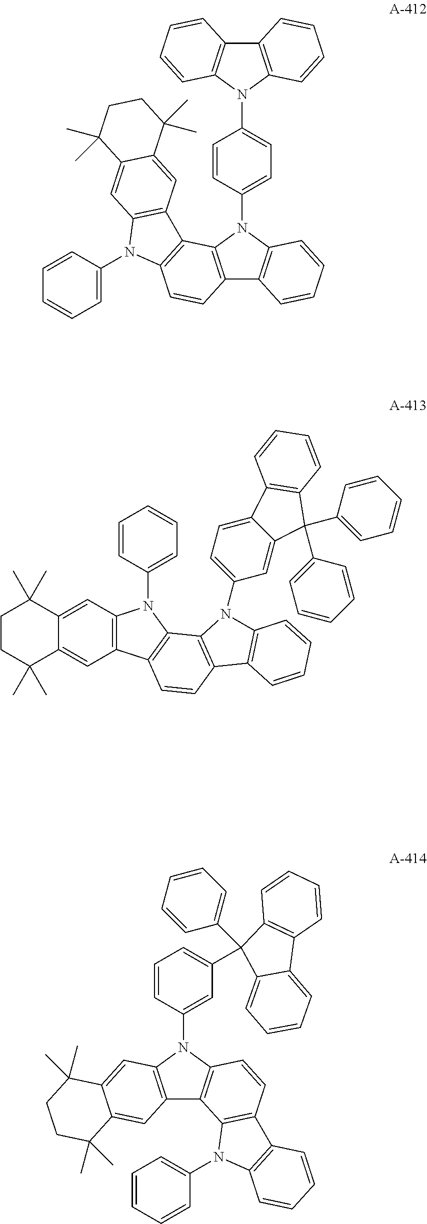 [SMILES: CC1(C)CCC(C)(C)c2cc3c(cc21)c1c(ccc2c4ccccc4n(-c4ccc(-n5c6ccccc6c6ccccc65)cc4)c21)n3-c1ccccc1 ; CC1(C)CCC(C)(C)c2cc3c(cc21)c1ccc2c4ccccc4n(-c4ccc5c(c4)C(c4ccccc4)(c4ccccc4)c4ccccc4-5)c2c1n3-c1ccccc1 ; CC1(C)CCC(C)(C)c2cc3c(cc21)c1c(ccc2c4ccccc4n(-c4ccccc4)c21)n3-c1cccc(C2(c3ccccc3)c3ccccc3-c3ccccc32)c1]

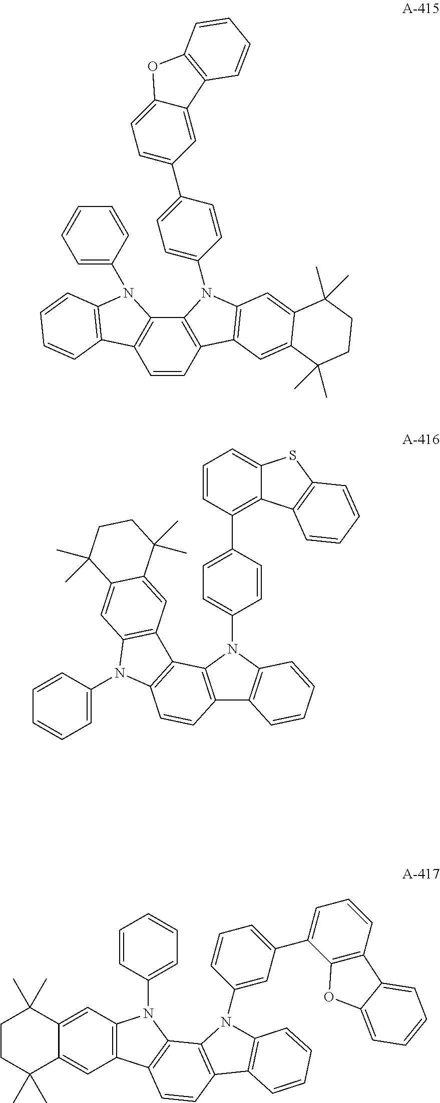 [SMILES: CC1(C)CCC(C)(C)c2cc3c(cc21)c1ccc2c4ccccc4n(-c4ccccc4)c2c1n3-c1ccc(-c2ccc3oc4ccccc4c3c2)cc1 ; CC1(C)CCC(C)(C)c2cc3c(cc21)c1c(ccc2c4ccccc4n(-c4ccc(-c5cccc6sc7ccccc7c56)cc4)c21)n3-c1ccccc1 ; CC1(C)CCC(C)(C)c2cc3c(cc21)c1ccc2c4ccccc4n(-c4cccc(-c5cccc6c5oc5ccccc56)c4)c2c1n3-c1ccccc1]

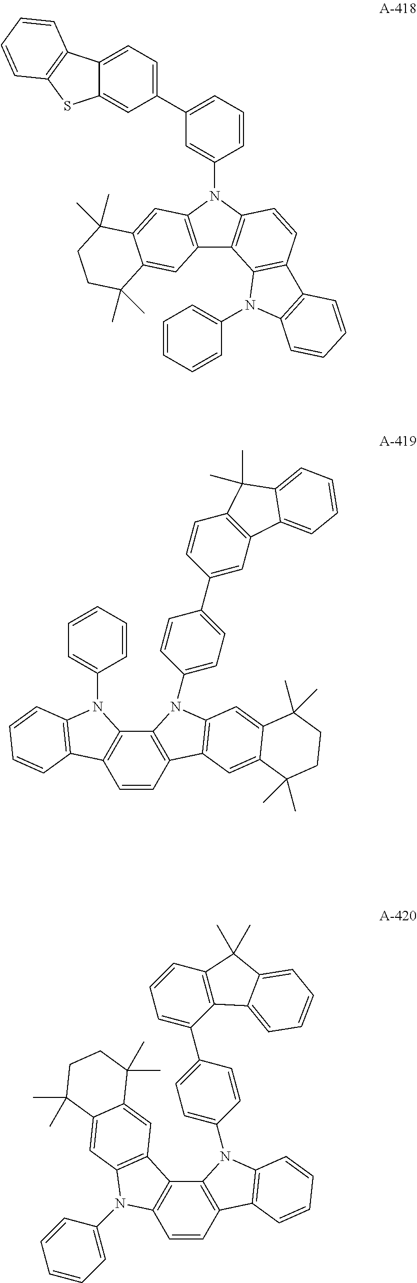 [SMILES: CC1(C)CCC(C)(C)c2cc3c(cc21)c1c(ccc2c4ccccc4n(-c4ccccc4)c21)n3-c1cccc(-c2ccc3c(c2)sc2ccccc23)c1 ; CC1(C)CCC(C)(C)c2cc3c(cc21)c1ccc2c4ccccc4n(-c4ccccc4)c2c1n3-c1ccc(-c2ccc3c(c2)-c2ccccc2C3(C)C)cc1 ; CC1(C)CCC(C)(C)c2cc3c(cc21)c1c(ccc2c4ccccc4n(-c4ccc(-c5cccc6c5-c5ccccc5C6(C)C)cc4)c21)n3-c1ccccc1]

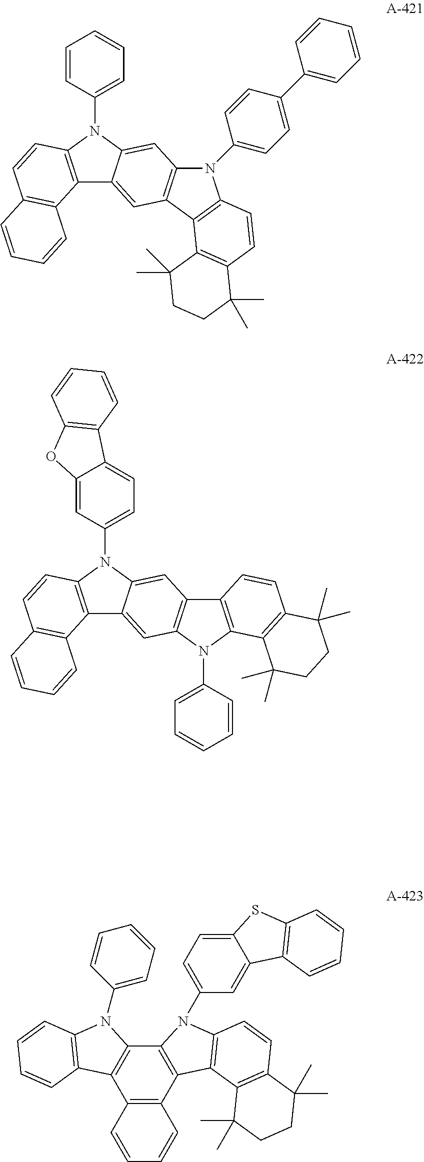 [SMILES: CC1(C)CCC(C)(C)c2c1ccc1c2c2cc3c4c5ccccc5ccc4n(-c4ccccc4)c3cc2n1-c1ccc(-c2ccccc2)cc1 ; CC1(C)CCC(C)(C)c2c1ccc1c3cc4c(cc3n(-c3ccccc3)c21)c1c2ccccc2ccc1n4-c1ccc2c(c1)oc1ccccc12 ; CC1(C)CCC(C)(C)c2c1ccc1c2c2c3ccccc3c3c4ccccc4n(-c4ccccc4)c3c2n1-c1ccc2sc3ccccc3c2c1]

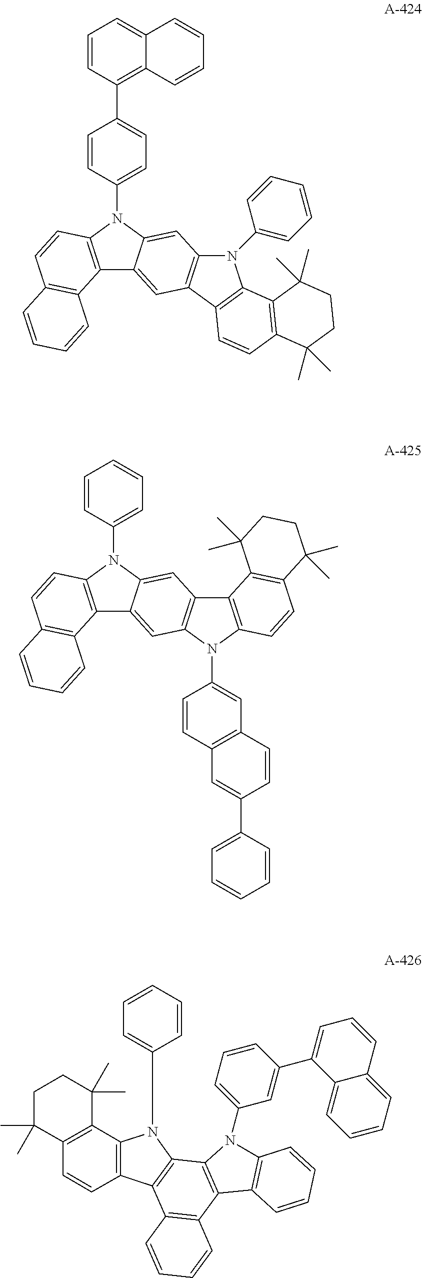

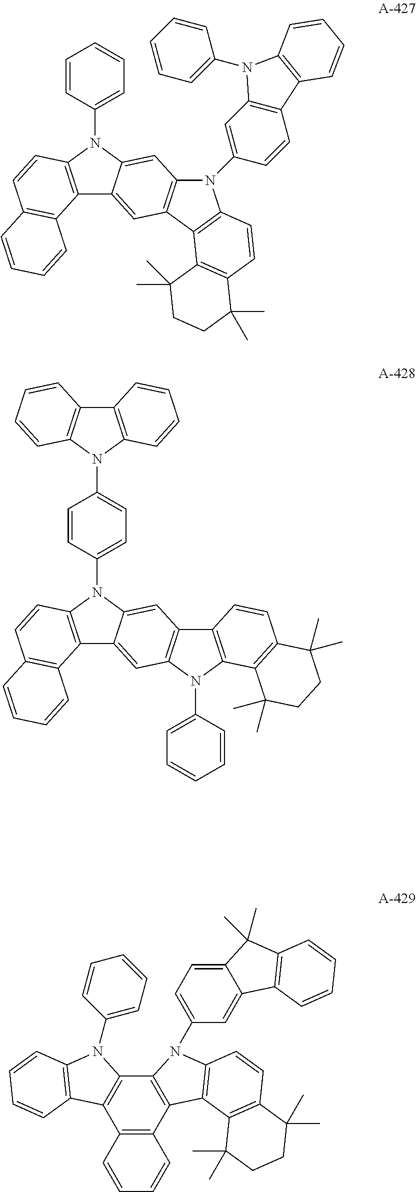 [SMILES: CC1(C)CCC(C)(C)c2c1ccc1c2c2cc3c4c5ccccc5ccc4n(-c4ccccc4)c3cc2n1-c1ccc2c3ccccc3n(-c3ccccc3)c2c1 ; CC1(C)CCC(C)(C)c2c1ccc1c3cc4c(cc3n(-c3ccccc3)c21)c1c2ccccc2ccc1n4-c1ccc(-n2c3ccccc3c3ccccc32)cc1 ; CC1(C)CCC(C)(C)c2c1ccc1c2c2c3ccccc3c3c4ccccc4n(-c4ccccc4)c3c2n1-c1ccc2c(c1)-c1ccccc1C2(C)C]

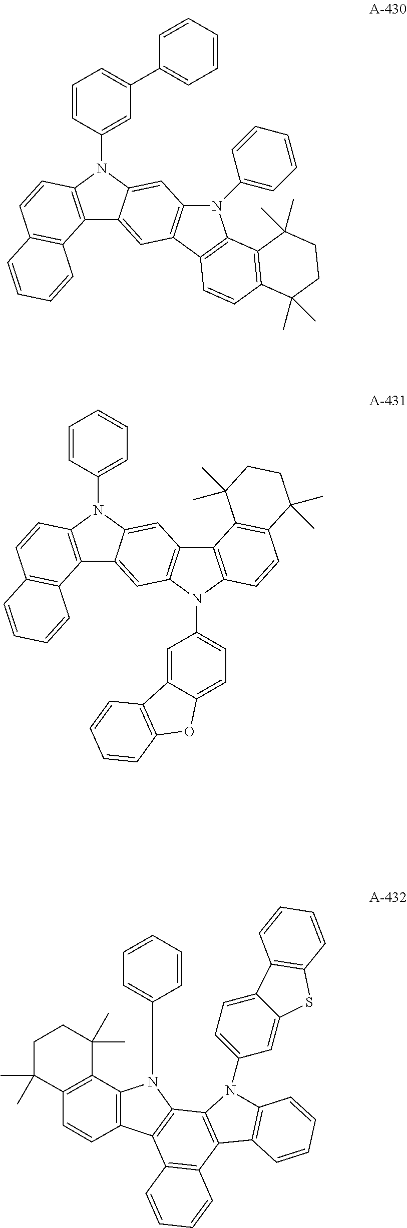

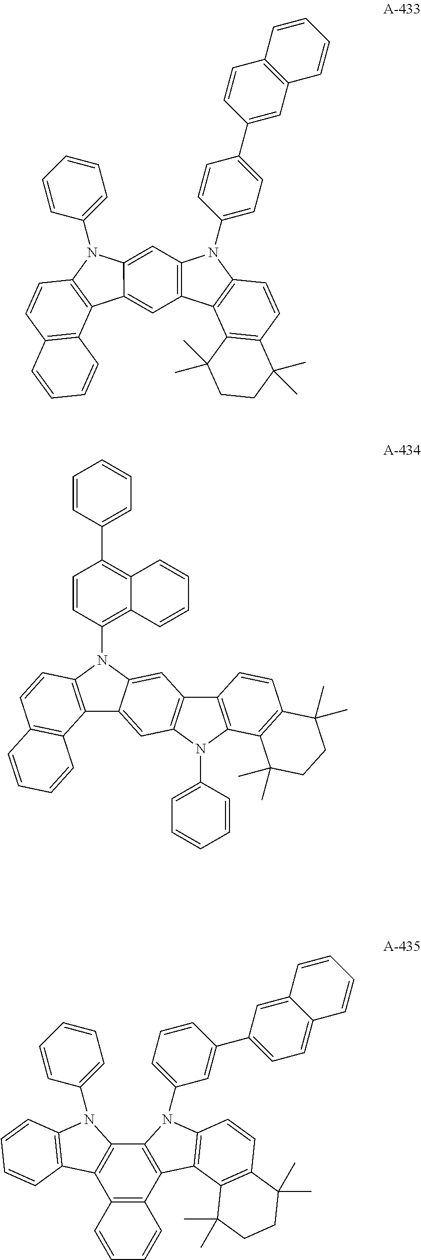 [SMILES: CC1(C)CCC(C)(C)c2c1ccc1c2c2cc3c4c5ccccc5ccc4n(-c4ccccc4)c3cc2n1-c1ccc(-c2ccc3ccccc3c2)cc1 ; CC1(C)CCC(C)(C)c2c1ccc1c3cc4c(cc3n(-c3ccccc3)c21)c1c2ccccc2ccc1n4-c1ccc(-c2ccccc2)c2ccccc12 ; CC1(C)CCC(C)(C)c2c1ccc1c2c2c3ccccc3c3c4ccccc4n(-c4ccccc4)c3c2n1-c1cccc(-c2ccc3ccccc3c2)c1]

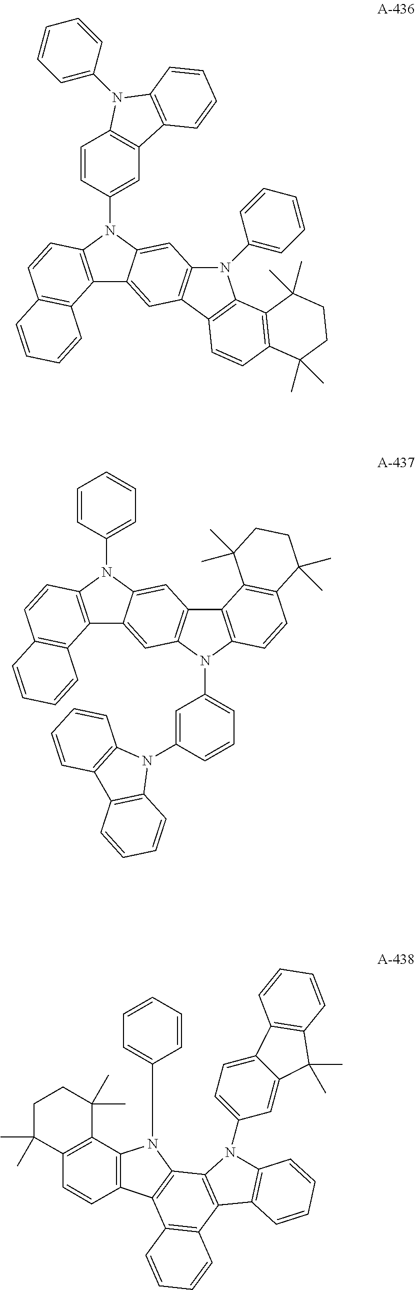

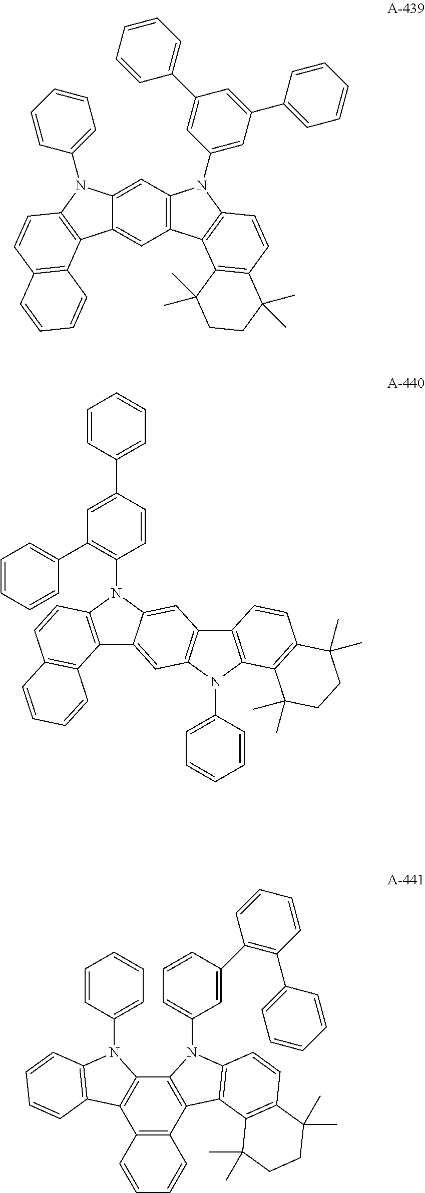 [SMILES: CC1(C)CCC(C)(C)c2c1ccc1c2c2cc3c4c5ccccc5ccc4n(-c4ccccc4)c3cc2n1-c1cc(-c2ccccc2)cc(-c2ccccc2)c1 ; CC1(C)CCC(C)(C)c2c1ccc1c3cc4c(cc3n(-c3ccccc3)c21)c1c2ccccc2ccc1n4-c1ccc(-c2ccccc2)cc1-c1ccccc1 ; CC1(C)CCC(C)(C)c2c1ccc1c2c2c3ccccc3c3c4ccccc4n(-c4ccccc4)c3c2n1-c1cccc(-c2ccccc2-c2ccccc2)c1]

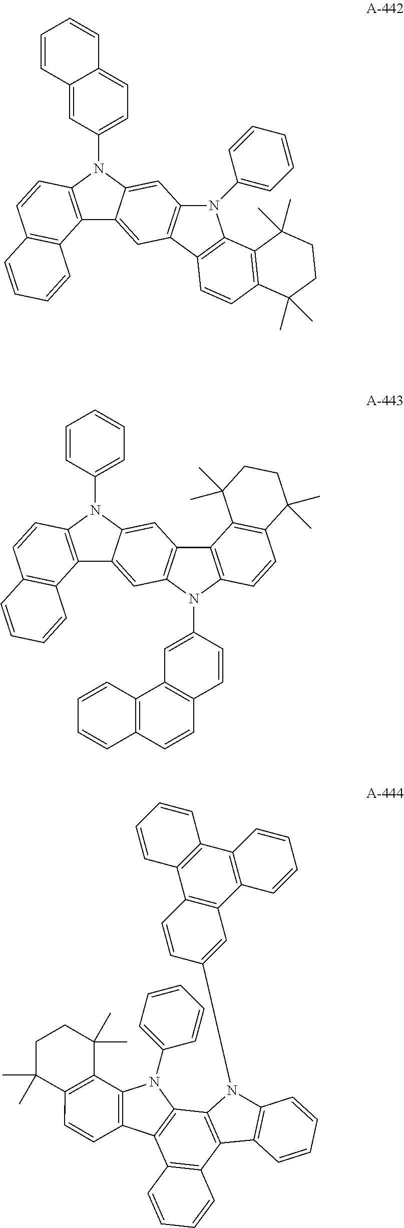

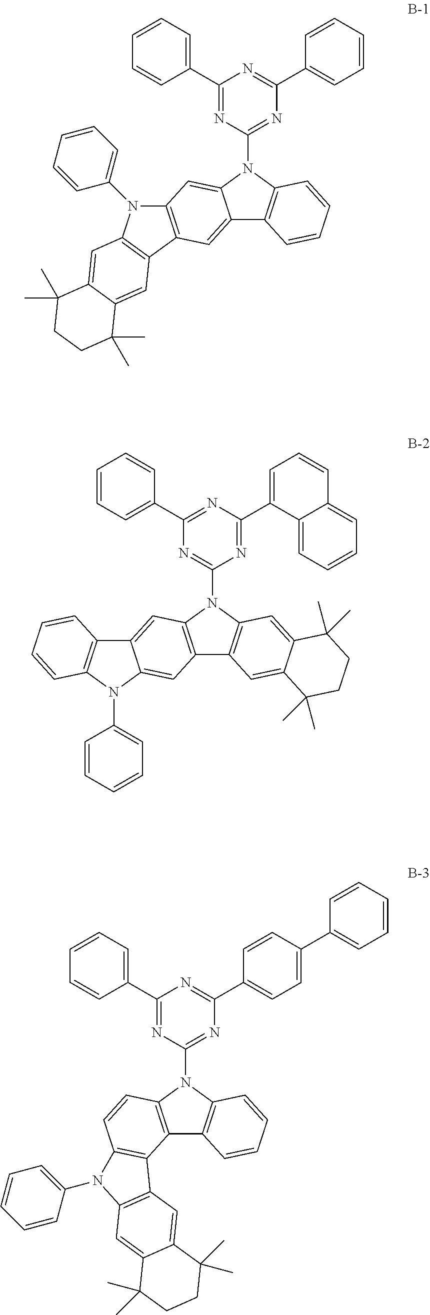 [SMILES: CC1(C)CCC(C)(C)c2cc3c(cc21)c1cc2c4ccccc4n(-c4nc(-c5ccccc5)nc(-c5ccccc5)n4)c2cc1n3-c1ccccc1 ; CC1(C)CCC(C)(C)c2cc3c(cc21)c1cc2c(cc1n3-c1nc(-c3ccccc3)nc(-c3cccc4ccccc34)n1)c1ccccc1n2-c1ccccc1 ; CC1(C)CCC(C)(C)c2cc3c(cc21)c1c2c4ccccc4n(-c4nc(-c5ccccc5)nc(-c5ccc(-c6ccccc6)cc5)n4)c2ccc1n3-c1ccccc1]

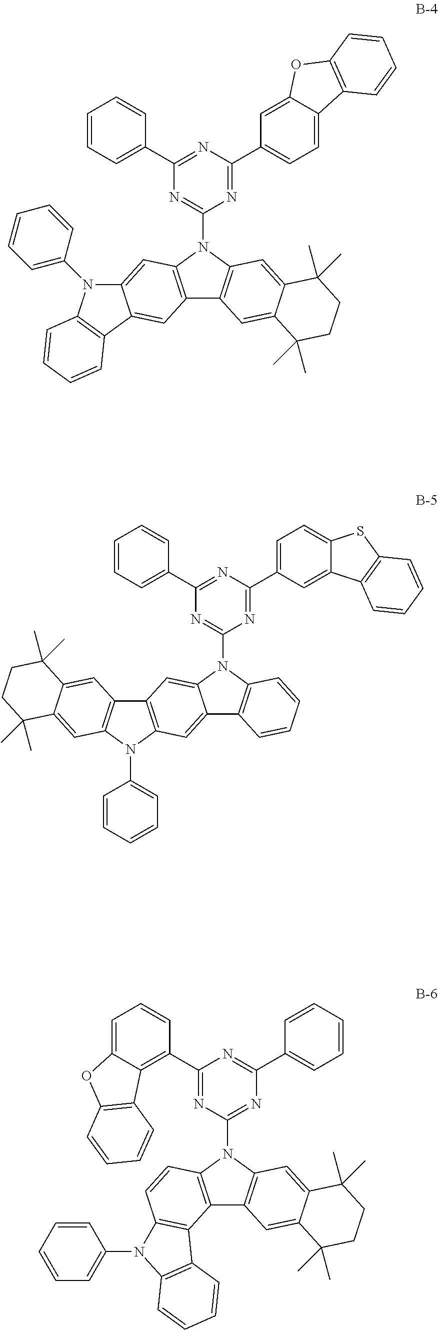 [SMILES: CC1(C)CCC(C)(C)c2cc3c(cc21)c1cc2c4ccccc4n(-c4ccccc4)c2cc1n3-c1nc(-c2ccccc2)nc(-c2ccc3c(c2)oc2ccccc23)n1 ; CC1(C)CCC(C)(C)c2cc3c(cc21)c1cc2c(cc1n3-c1ccccc1)c1ccccc1n2-c1nc(-c2ccccc2)nc(-c2ccc3sc4ccccc4c3c2)n1 ; CC1(C)CCC(C)(C)c2cc3c(cc21)c1c2c4ccccc4n(-c4ccccc4)c2ccc1n3-c1nc(-c2ccccc2)nc(-c2cccc3oc4ccccc4c23)n1]

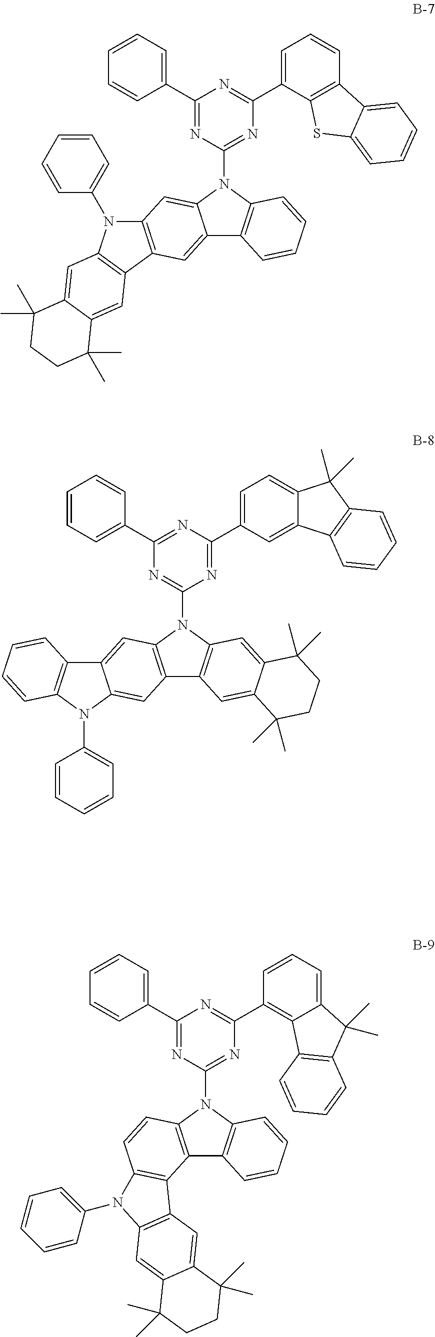 [SMILES: CC1(C)CCC(C)(C)c2cc3c(cc21)c1cc2c4ccccc4n(-c4nc(-c5ccccc5)nc(-c5cccc6c5sc5ccccc56)n4)c2cc1n3-c1ccccc1 ; CC1(C)CCC(C)(C)c2cc3c(cc21)c1cc2c(cc1n3-c1nc(-c3ccccc3)nc(-c3ccc4c(c3)-c3ccccc3C4(C)C)n1)c1ccccc1n2-c1ccccc1 ; CC1(C)CCC(C)(C)c2cc3c(cc21)c1c2c4ccccc4n(-c4nc(-c5ccccc5)nc(-c5cccc6c5-c5ccccc5C6(C)C)n4)c2ccc1n3-c1ccccc1]

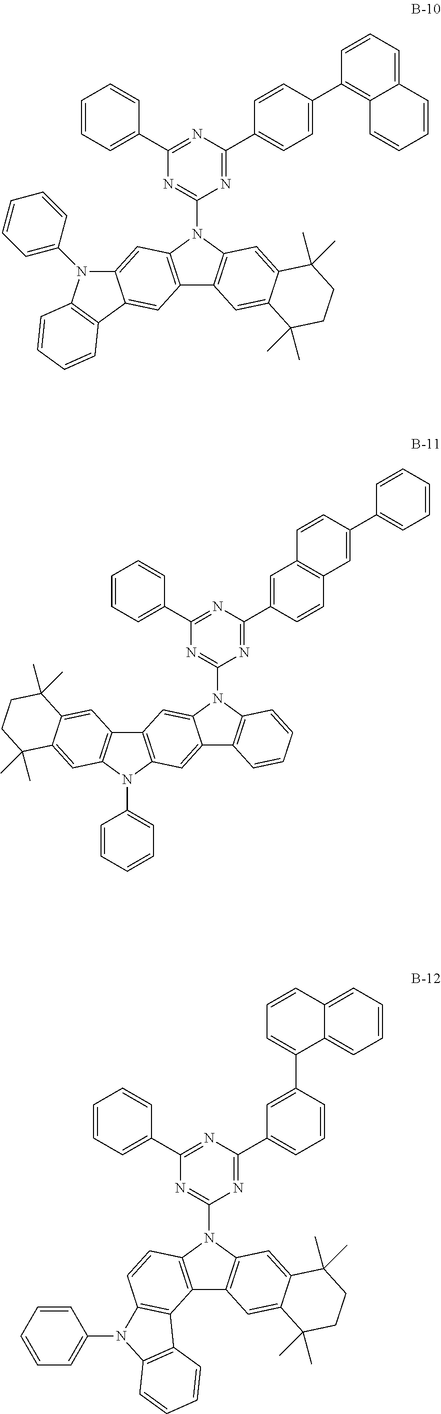 [SMILES: CC1(C)CCC(C)(C)c2cc3c(cc21)c1cc2c4ccccc4n(-c4ccccc4)c2cc1n3-c1nc(-c2ccccc2)nc(-c2ccc(-c3cccc4ccccc34)cc2)n1 ; CC1(C)CCC(C)(C)c2cc3c(cc21)c1cc2c(cc1n3-c1ccccc1)c1ccccc1n2-c1nc(-c2ccccc2)nc(-c2ccc3cc(-c4ccccc4)ccc3c2)n1 ; CC1(C)CCC(C)(C)c2cc3c(cc21)c1c2c4ccccc4n(-c4ccccc4)c2ccc1n3-c1nc(-c2ccccc2)nc(-c2cccc(-c3cccc4ccccc34)c2)n1]

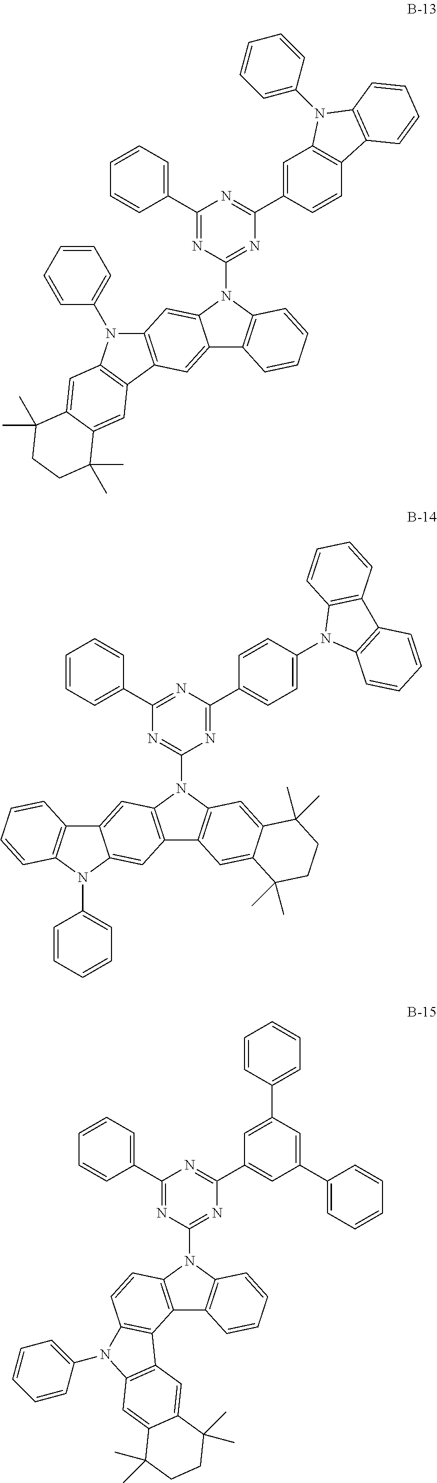 [SMILES: CC1(C)CCC(C)(C)c2cc3c(cc21)c1cc2c4ccccc4n(-c4nc(-c5ccccc5)nc(-c5ccc6c7ccccc7n(-c7ccccc7)c6c5)n4)c2cc1n3-c1ccccc1 ; CC1(C)CCC(C)(C)c2cc3c(cc21)c1cc2c(cc1n3-c1nc(-c3ccccc3)nc(-c3ccc(-n4c5ccccc5c5ccccc54)cc3)n1)c1ccccc1n2-c1ccccc1 ; CC1(C)CCC(C)(C)c2cc3c(cc21)c1c2c4ccccc4n(-c4nc(-c5ccccc5)nc(-c5cc(-c6ccccc6)cc(-c6ccccc6)c5)n4)c2ccc1n3-c1ccccc1]

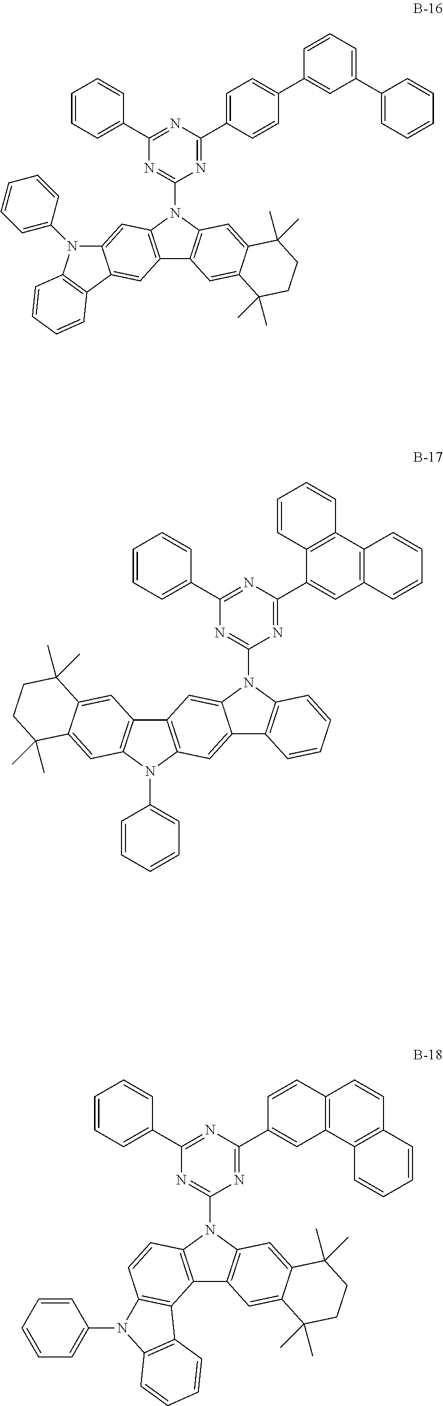 [SMILES: CC1(C)CCC(C)(C)c2cc3c(cc21)c1cc2c4ccccc4n(-c4ccccc4)c2cc1n3-c1nc(-c2ccccc2)nc(-c2ccc(-c3cccc(-c4ccccc4)c3)cc2)n1 ; CC1(C)CCC(C)(C)c2cc3c(cc21)c1cc2c(cc1n3-c1ccccc1)c1ccccc1n2-c1nc(-c2ccccc2)nc(-c2cc3ccccc3c3ccccc23)n1 ; CC1(C)CCC(C)(C)c2cc3c(cc21)c1c2c4ccccc4n(-c4ccccc4)c2ccc1n3-c1nc(-c2ccccc2)nc(-c2ccc3ccc4ccccc4c3c2)n1]

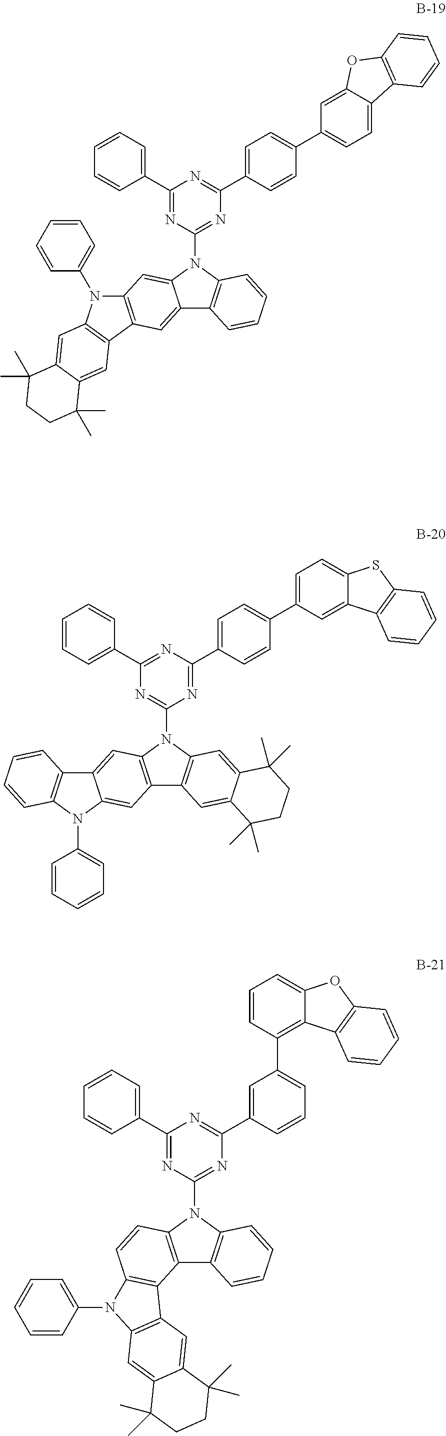 [SMILES: CC1(C)CCC(C)(C)c2cc3c(cc21)c1cc2c4ccccc4n(-c4nc(-c5ccccc5)nc(-c5ccc(-c6ccc7c(c6)oc6ccccc67)cc5)n4)c2cc1n3-c1ccccc1 ; CC1(C)CCC(C)(C)c2cc3c(cc21)c1cc2c(cc1n3-c1nc(-c3ccccc3)nc(-c3ccc(-c4ccc5sc6ccccc6c5c4)cc3)n1)c1ccccc1n2-c1ccccc1 ; CC1(C)CCC(C)(C)c2cc3c(cc21)c1c2c4ccccc4n(-c4nc(-c5ccccc5)nc(-c5cccc(-c6cccc7oc8ccccc8c67)c5)n4)c2ccc1n3-c1ccccc1]

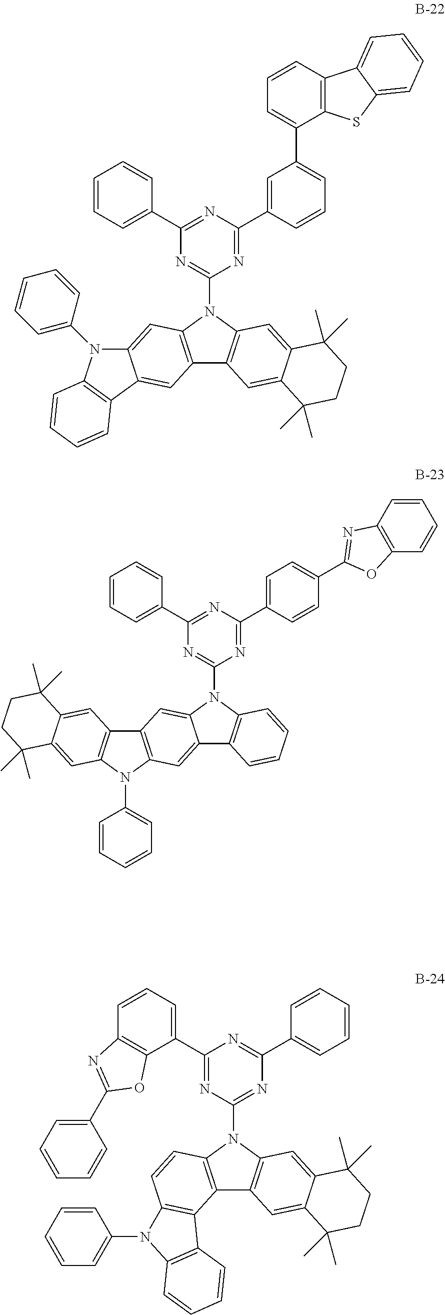 [SMILES: CC1(C)CCC(C)(C)c2cc3c(cc21)c1cc2c4ccccc4n(-c4ccccc4)c2cc1n3-c1nc(-c2ccccc2)nc(-c2cccc(-c3cccc4c3sc3ccccc34)c2)n1 ; CC1(C)CCC(C)(C)c2cc3c(cc21)c1cc2c(cc1n3-c1ccccc1)c1ccccc1n2-c1nc(-c2ccccc2)nc(-c2ccc(-c3nc4ccccc4o3)cc2)n1 ; CC1(C)CCC(C)(C)c2cc3c(cc21)c1c2c4ccccc4n(-c4ccccc4)c2ccc1n3-c1nc(-c2ccccc2)nc(-c2cccc3nc(-c4ccccc4)oc23)n1]

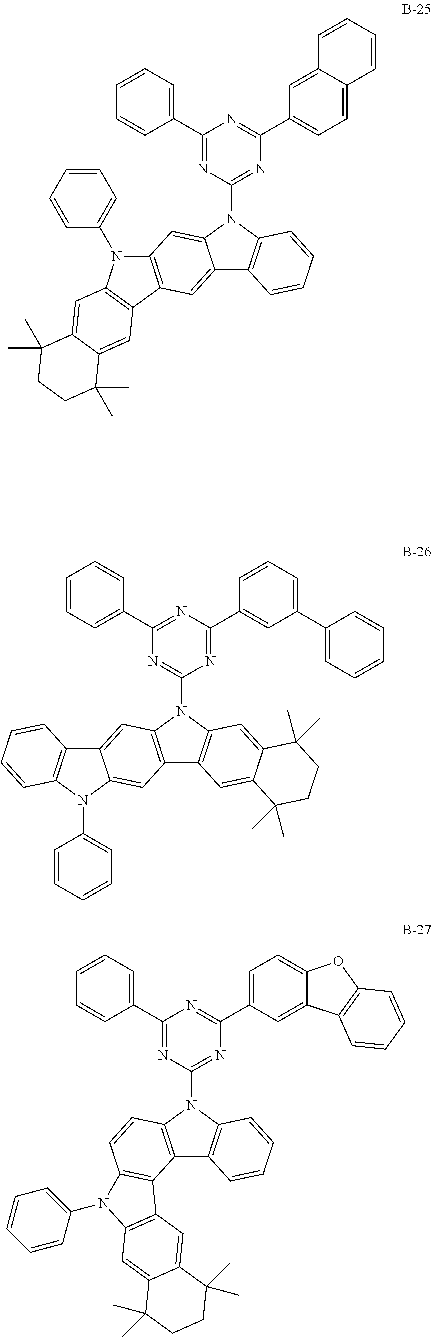 [SMILES: CC1(C)CCC(C)(C)c2cc3c(cc21)c1cc2c4ccccc4n(-c4nc(-c5ccccc5)nc(-c5ccc6ccccc6c5)n4)c2cc1n3-c1ccccc1 ; CC1(C)CCC(C)(C)c2cc3c(cc21)c1cc2c(cc1n3-c1nc(-c3ccccc3)nc(-c3cccc(-c4ccccc4)c3)n1)c1ccccc1n2-c1ccccc1 ; CC1(C)CCC(C)(C)c2cc3c(cc21)c1c2c4ccccc4n(-c4nc(-c5ccccc5)nc(-c5ccc6oc7ccccc7c6c5)n4)c2ccc1n3-c1ccccc1]

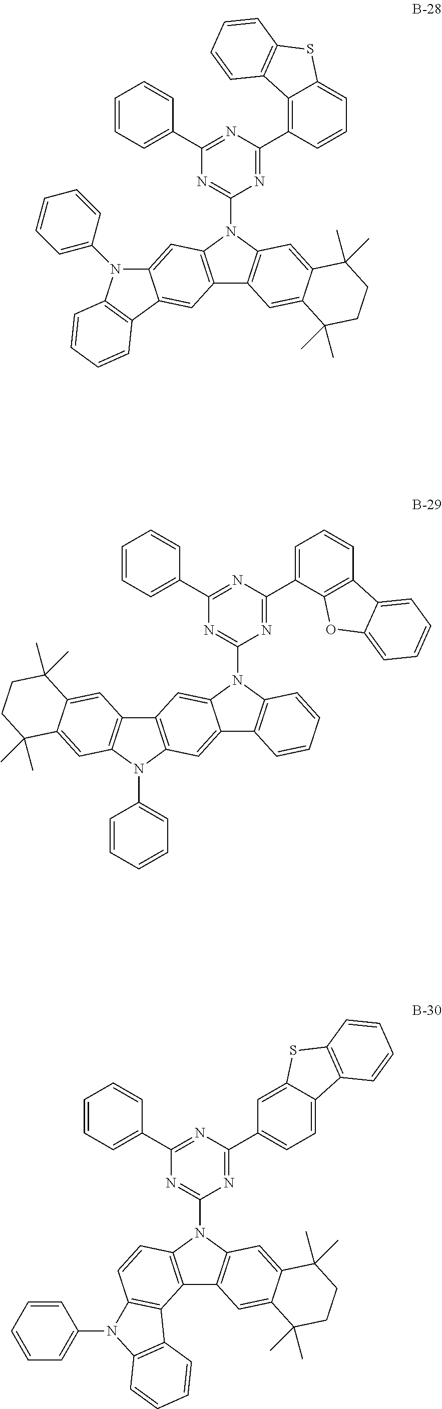

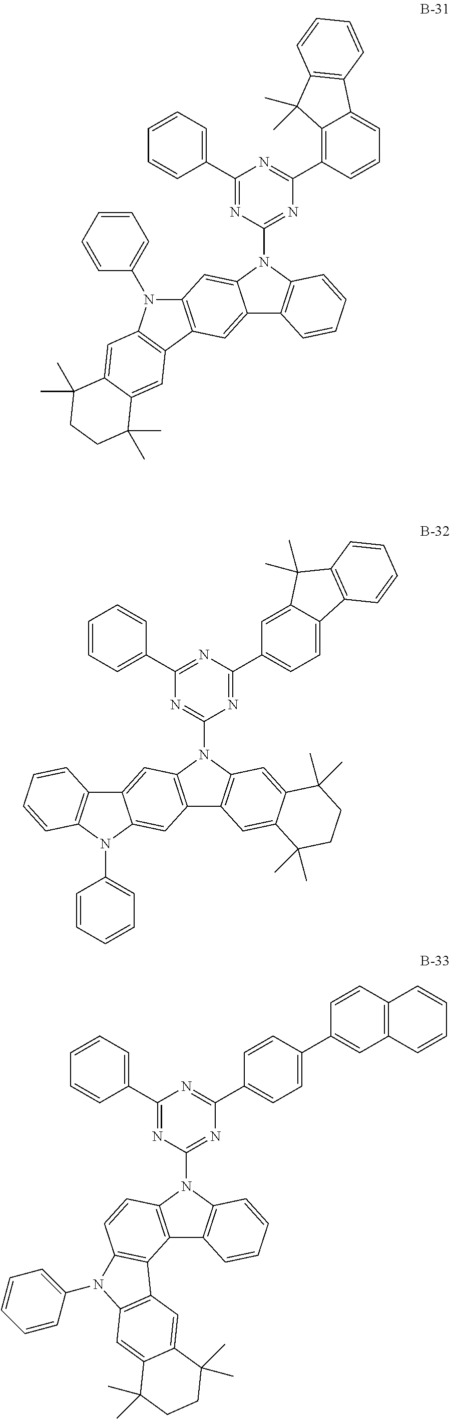 [SMILES: CC1(C)CCC(C)(C)c2cc3c(cc21)c1cc2c4ccccc4n(-c4nc(-c5ccccc5)nc(-c5cccc6c5C(C)(C)c5ccccc5-6)n4)c2cc1n3-c1ccccc1 ; CC1(C)CCC(C)(C)c2cc3c(cc21)c1cc2c(cc1n3-c1nc(-c3ccccc3)nc(-c3ccc4c(c3)C(C)(C)c3ccccc3-4)n1)c1ccccc1n2-c1ccccc1 ; CC1(C)CCC(C)(C)c2cc3c(cc21)c1c2c4ccccc4n(-c4nc(-c5ccccc5)nc(-c5ccc(-c6ccc7ccccc7c6)cc5)n4)c2ccc1n3-c1ccccc1]

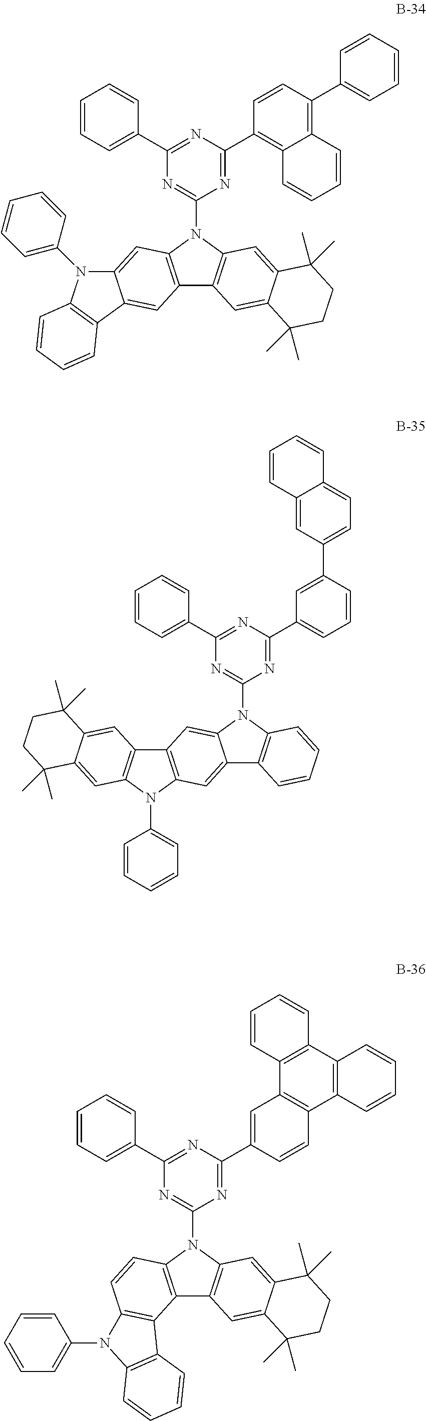 [SMILES: CC1(C)CCC(C)(C)c2cc3c(cc21)c1cc2c4ccccc4n(-c4ccccc4)c2cc1n3-c1nc(-c2ccccc2)nc(-c2ccc(-c3ccccc3)c3ccccc23)n1 ; CC1(C)CCC(C)(C)c2cc3c(cc21)c1cc2c(cc1n3-c1ccccc1)c1ccccc1n2-c1nc(-c2ccccc2)nc(-c2cccc(-c3ccc4ccccc4c3)c2)n1 ; CC1(C)CCC(C)(C)c2cc3c(cc21)c1c2c4ccccc4n(-c4ccccc4)c2ccc1n3-c1nc(-c2ccccc2)nc(-c2ccc3c4ccccc4c4ccccc4c3c2)n1]

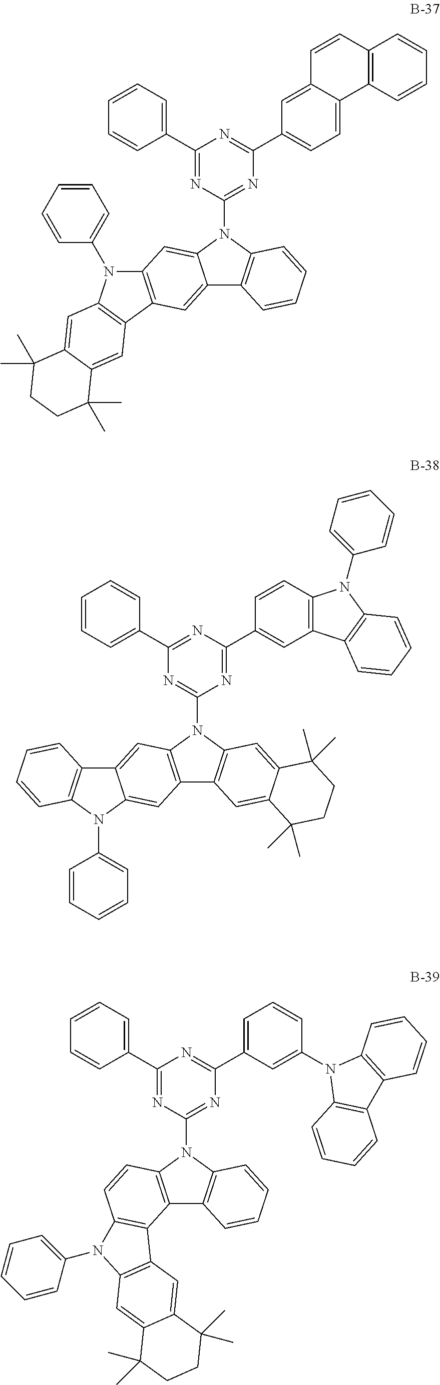 [SMILES: CC1(C)CCC(C)(C)c2cc3c(cc21)c1cc2c4ccccc4n(-c4nc(-c5ccccc5)nc(-c5ccc6c(ccc7ccccc76)c5)n4)c2cc1n3-c1ccccc1 ; CC1(C)CCC(C)(C)c2cc3c(cc21)c1cc2c(cc1n3-c1nc(-c3ccccc3)nc(-c3ccc4c(c3)c3ccccc3n4-c3ccccc3)n1)c1ccccc1n2-c1ccccc1 ; CC1(C)CCC(C)(C)c2cc3c(cc21)c1c2c4ccccc4n(-c4nc(-c5ccccc5)nc(-c5cccc(-n6c7ccccc7c7ccccc76)c5)n4)c2ccc1n3-c1ccccc1]

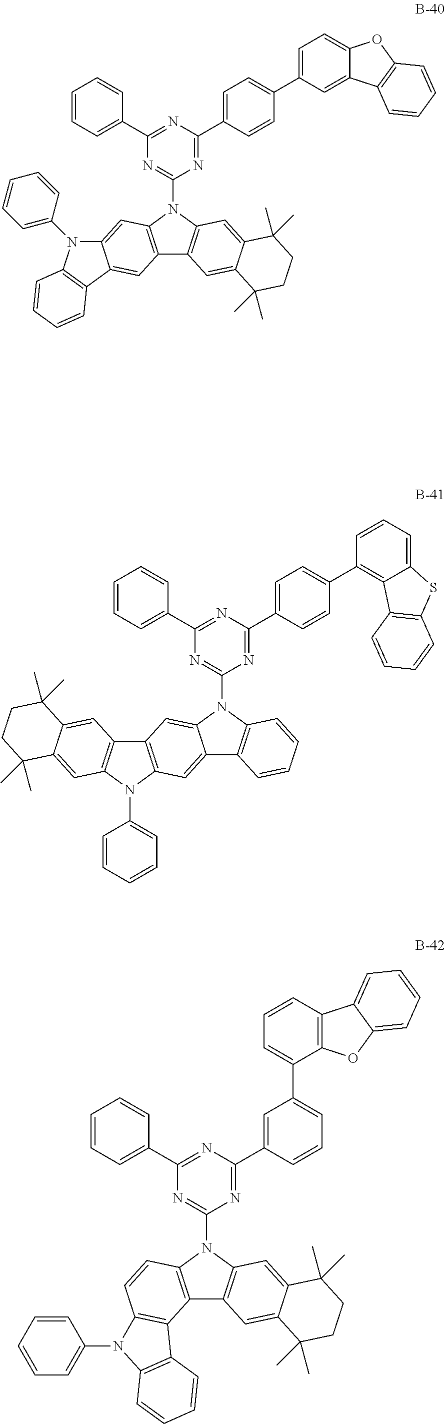 [SMILES: CC1(C)CCC(C)(C)c2cc3c(cc21)c1cc2c4ccccc4n(-c4ccccc4)c2cc1n3-c1nc(-c2ccccc2)nc(-c2ccc(-c3ccc4oc5ccccc5c4c3)cc2)n1 ; CC1(C)CCC(C)(C)c2cc3c(cc21)c1cc2c(cc1n3-c1ccccc1)c1ccccc1n2-c1nc(-c2ccccc2)nc(-c2ccc(-c3cccc4sc5ccccc5c34)cc2)n1 ; CC1(C)CCC(C)(C)c2cc3c(cc21)c1c2c4ccccc4n(-c4ccccc4)c2ccc1n3-c1nc(-c2ccccc2)nc(-c2cccc(-c3cccc4c3oc3ccccc34)c2)n1]

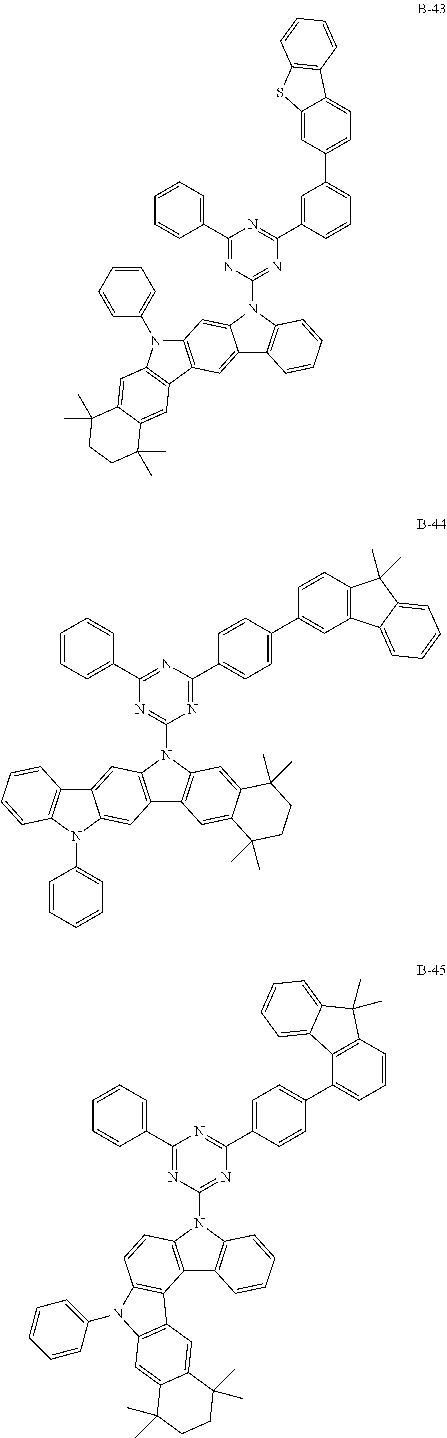 [SMILES: CC1(C)CCC(C)(C)c2cc3c(cc21)c1cc2c4ccccc4n(-c4nc(-c5ccccc5)nc(-c5cccc(-c6ccc7c(c6)sc6ccccc67)c5)n4)c2cc1n3-c1ccccc1 ; CC1(C)CCC(C)(C)c2cc3c(cc21)c1cc2c(cc1n3-c1nc(-c3ccccc3)nc(-c3ccc(-c4ccc5c(c4)-c4ccccc4C5(C)C)cc3)n1)c1ccccc1n2-c1ccccc1 ; CC1(C)CCC(C)(C)c2cc3c(cc21)c1c2c4ccccc4n(-c4nc(-c5ccccc5)nc(-c5ccc(-c6cccc7c6-c6ccccc6C7(C)C)cc5)n4)c2ccc1n3-c1ccccc1]

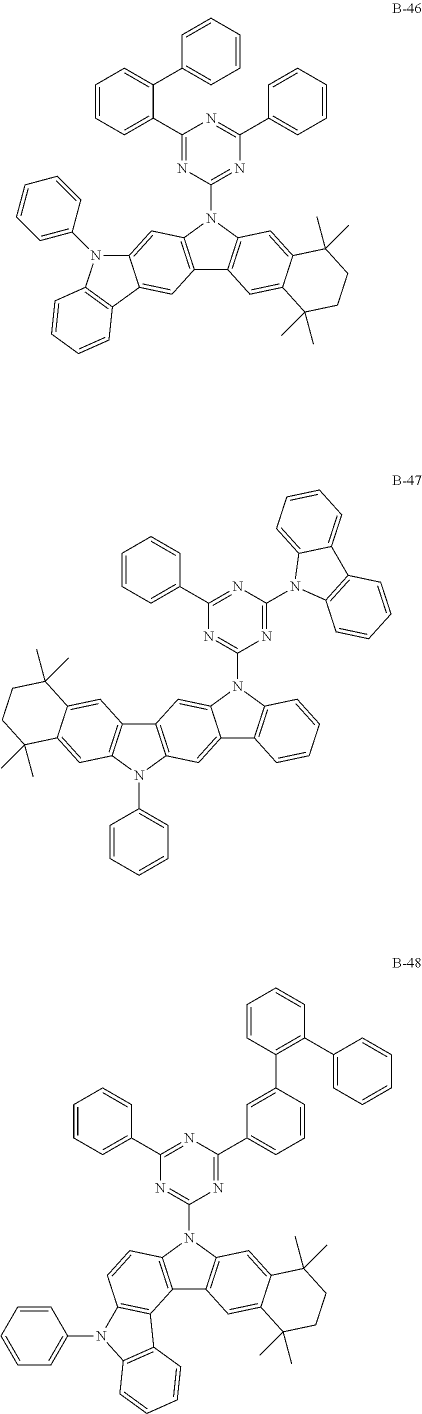

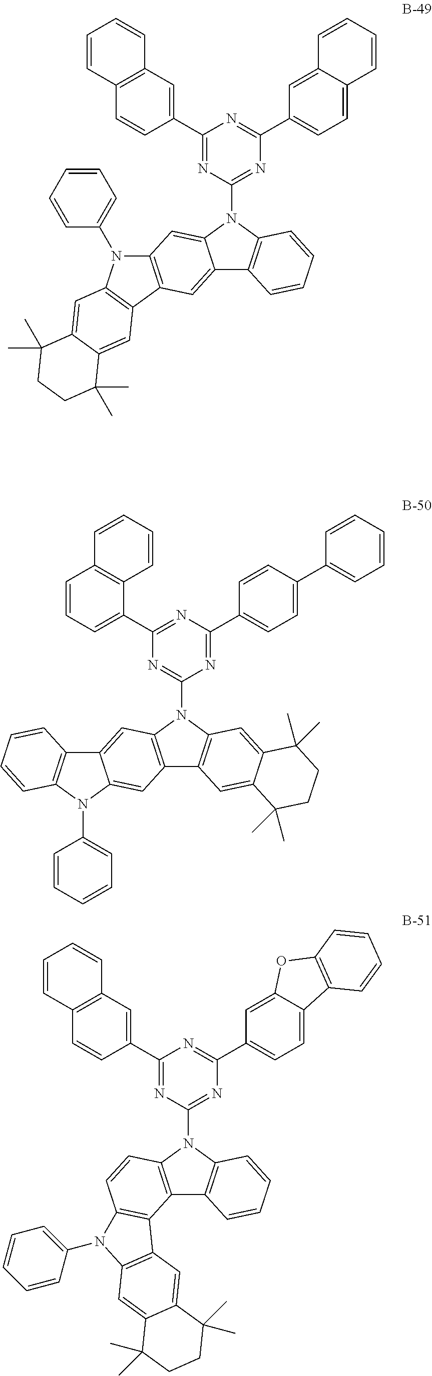

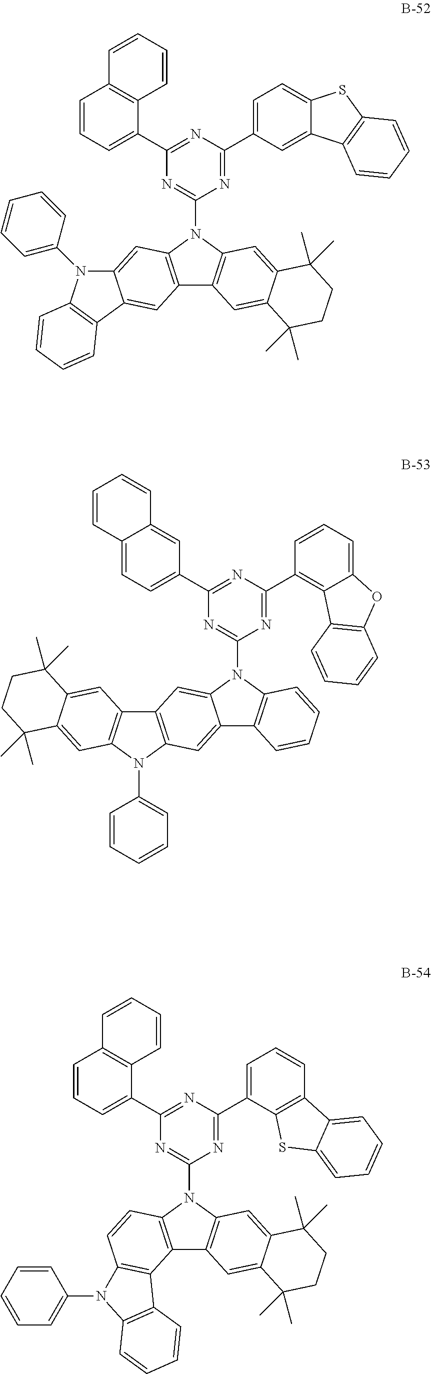 [SMILES: CC1(C)CCC(C)(C)c2cc3c(cc21)c1cc2c4ccccc4n(-c4ccccc4)c2cc1n3-c1nc(-c2ccc3sc4ccccc4c3c2)nc(-c2cccc3ccccc23)n1 ; CC1(C)CCC(C)(C)c2cc3c(cc21)c1cc2c(cc1n3-c1ccccc1)c1ccccc1n2-c1nc(-c2ccc3ccccc3c2)nc(-c2cccc3oc4ccccc4c23)n1 ; CC1(C)CCC(C)(C)c2cc3c(cc21)c1c2c4ccccc4n(-c4ccccc4)c2ccc1n3-c1nc(-c2cccc3ccccc23)nc(-c2cccc3c2sc2ccccc23)n1]

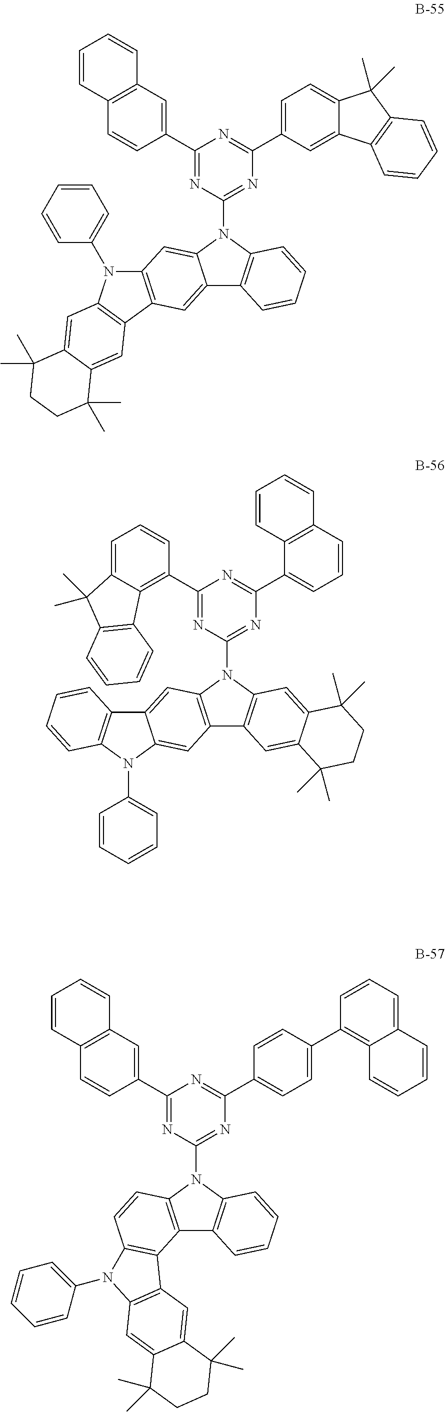 [SMILES: CC1(C)CCC(C)(C)c2cc3c(cc21)c1cc2c4ccccc4n(-c4nc(-c5ccc6c(c5)-c5ccccc5C6(C)C)nc(-c5ccc6ccccc6c5)n4)c2cc1n3-c1ccccc1 ; CC1(C)CCC(C)(C)c2cc3c(cc21)c1cc2c(cc1n3-c1nc(-c3cccc4c3-c3ccccc3C4(C)C)nc(-c3cccc4ccccc34)n1)c1ccccc1n2-c1ccccc1 ; CC1(C)CCC(C)(C)c2cc3c(cc21)c1c2c4ccccc4n(-c4nc(-c5ccc(-c6cccc7ccccc67)cc5)nc(-c5ccc6ccccc6c5)n4)c2ccc1n3-c1ccccc1]

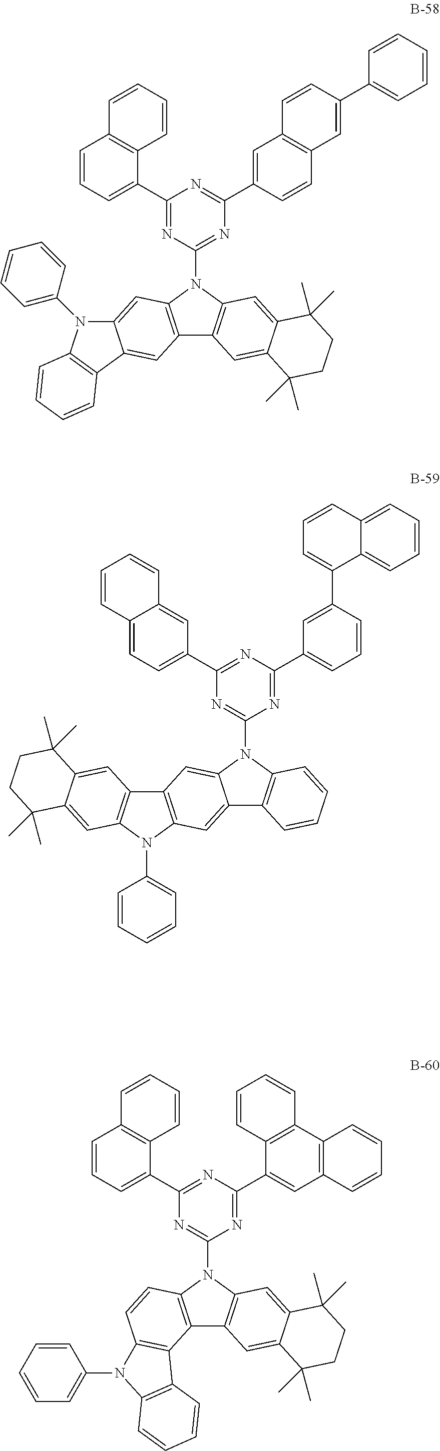 [SMILES: CC1(C)CCC(C)(C)c2cc3c(cc21)c1cc2c4ccccc4n(-c4ccccc4)c2cc1n3-c1nc(-c2ccc3cc(-c4ccccc4)ccc3c2)nc(-c2cccc3ccccc23)n1 ; CC1(C)CCC(C)(C)c2cc3c(cc21)c1cc2c(cc1n3-c1ccccc1)c1ccccc1n2-c1nc(-c2cccc(-c3cccc4ccccc34)c2)nc(-c2ccc3ccccc3c2)n1 ; CC1(C)CCC(C)(C)c2cc3c(cc21)c1c2c4ccccc4n(-c4ccccc4)c2ccc1n3-c1nc(-c2cccc3ccccc23)nc(-c2cc3ccccc3c3ccccc23)n1]

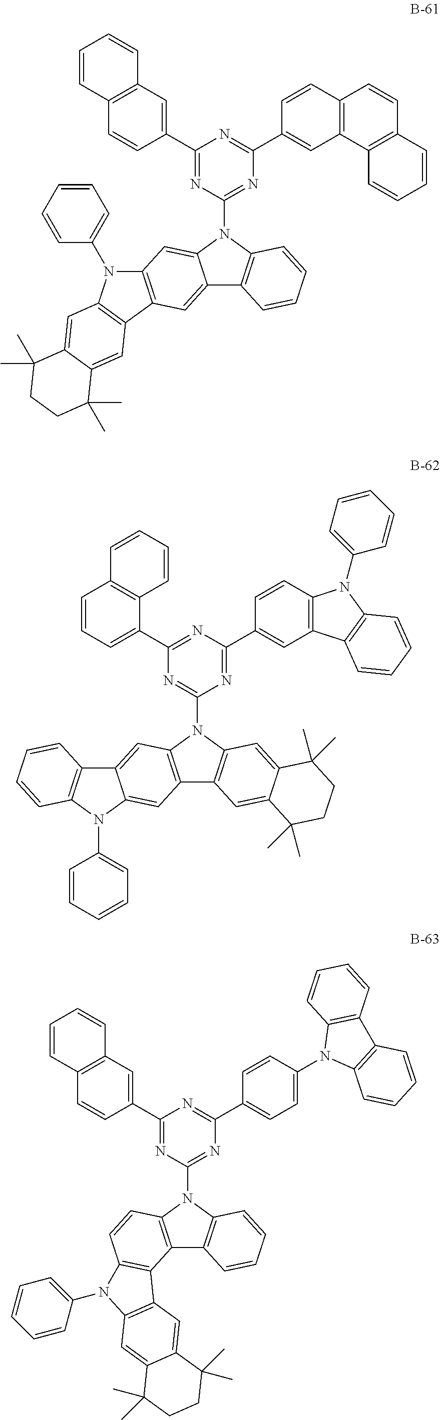 [SMILES: CC1(C)CCC(C)(C)c2cc3c(cc21)c1cc2c4ccccc4n(-c4nc(-c5ccc6ccccc6c5)nc(-c5ccc6ccc7ccccc7c6c5)n4)c2cc1n3-c1ccccc1 ; CC1(C)CCC(C)(C)c2cc3c(cc21)c1cc2c(cc1n3-c1nc(-c3ccc4c(c3)c3ccccc3n4-c3ccccc3)nc(-c3cccc4ccccc34)n1)c1ccccc1n2-c1ccccc1 ; CC1(C)CCC(C)(C)c2cc3c(cc21)c1c2c4ccccc4n(-c4nc(-c5ccc(-n6c7ccccc7c7ccccc76)cc5)nc(-c5ccc6ccccc6c5)n4)c2ccc1n3-c1ccccc1]

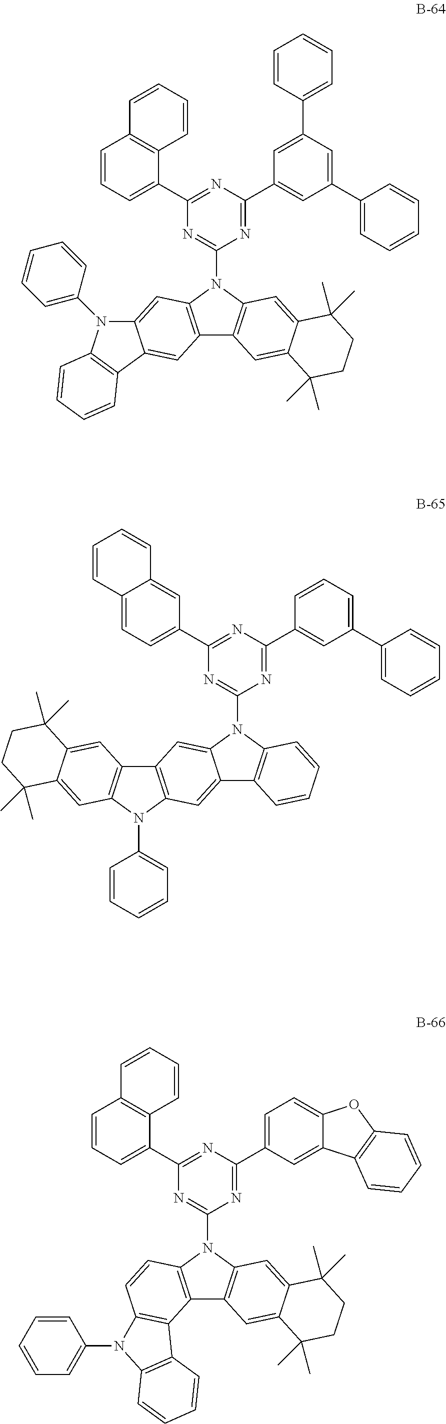 [SMILES: CC1(C)CCC(C)(C)c2cc3c(cc21)c1cc2c4ccccc4n(-c4ccccc4)c2cc1n3-c1nc(-c2cc(-c3ccccc3)cc(-c3ccccc3)c2)nc(-c2cccc3ccccc23)n1 ; CC1(C)CCC(C)(C)c2cc3c(cc21)c1cc2c(cc1n3-c1ccccc1)c1ccccc1n2-c1nc(-c2cccc(-c3ccccc3)c2)nc(-c2ccc3ccccc3c2)n1 ; CC1(C)CCC(C)(C)c2cc3c(cc21)c1c2c4ccccc4n(-c4ccccc4)c2ccc1n3-c1nc(-c2ccc3oc4ccccc4c3c2)nc(-c2cccc3ccccc23)n1]

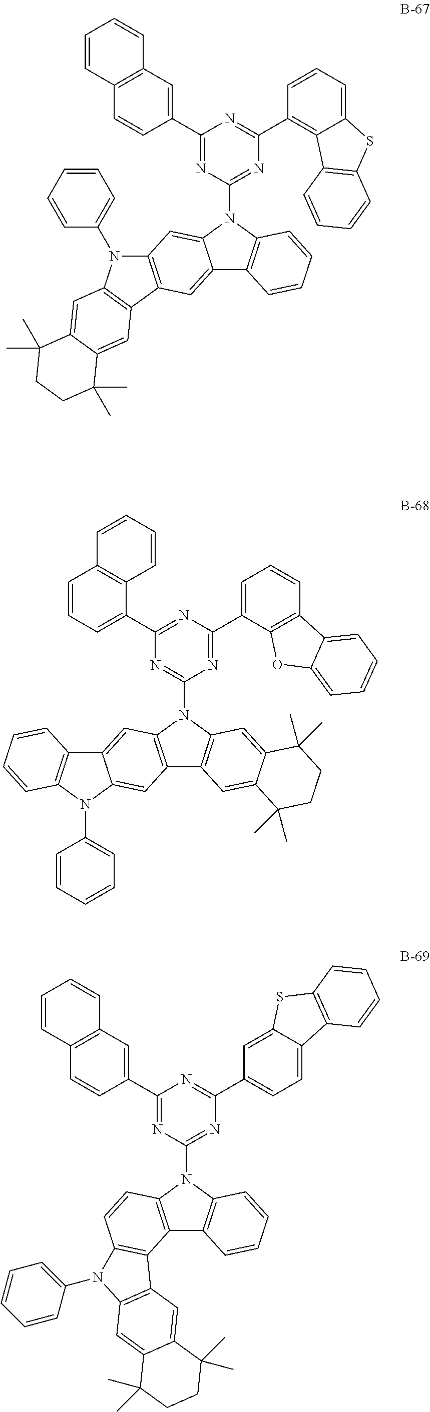 [SMILES: CC1(C)CCC(C)(C)c2cc3c(cc21)c1cc2c4ccccc4n(-c4nc(-c5ccc6ccccc6c5)nc(-c5cccc6sc7ccccc7c56)n4)c2cc1n3-c1ccccc1 ; CC1(C)CCC(C)(C)c2cc3c(cc21)c1cc2c(cc1n3-c1nc(-c3cccc4ccccc34)nc(-c3cccc4c3oc3ccccc34)n1)c1ccccc1n2-c1ccccc1 ; CC1(C)CCC(C)(C)c2cc3c(cc21)c1c2c4ccccc4n(-c4nc(-c5ccc6ccccc6c5)nc(-c5ccc6c(c5)sc5ccccc56)n4)c2ccc1n3-c1ccccc1]

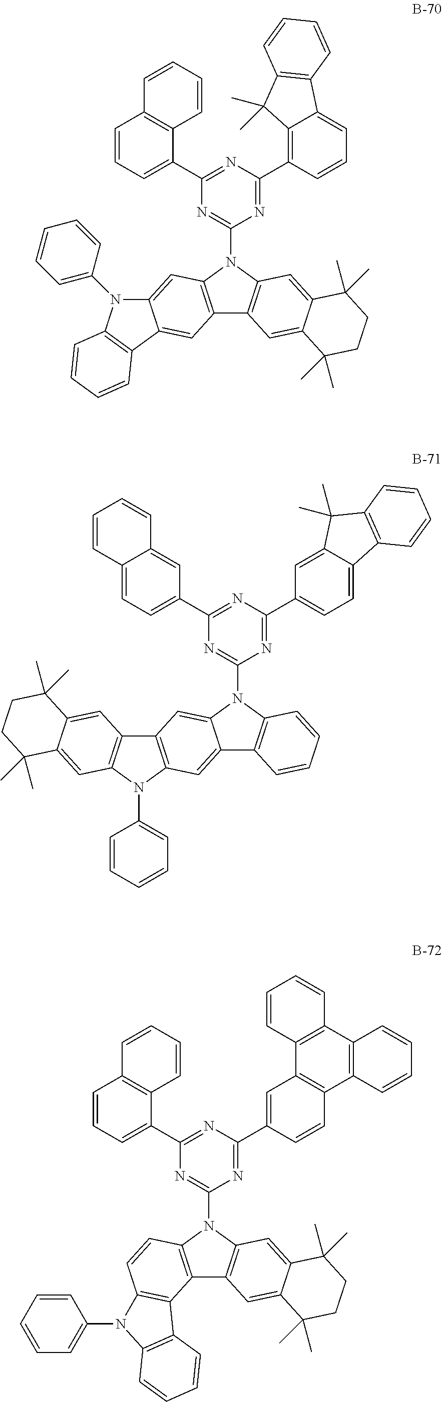

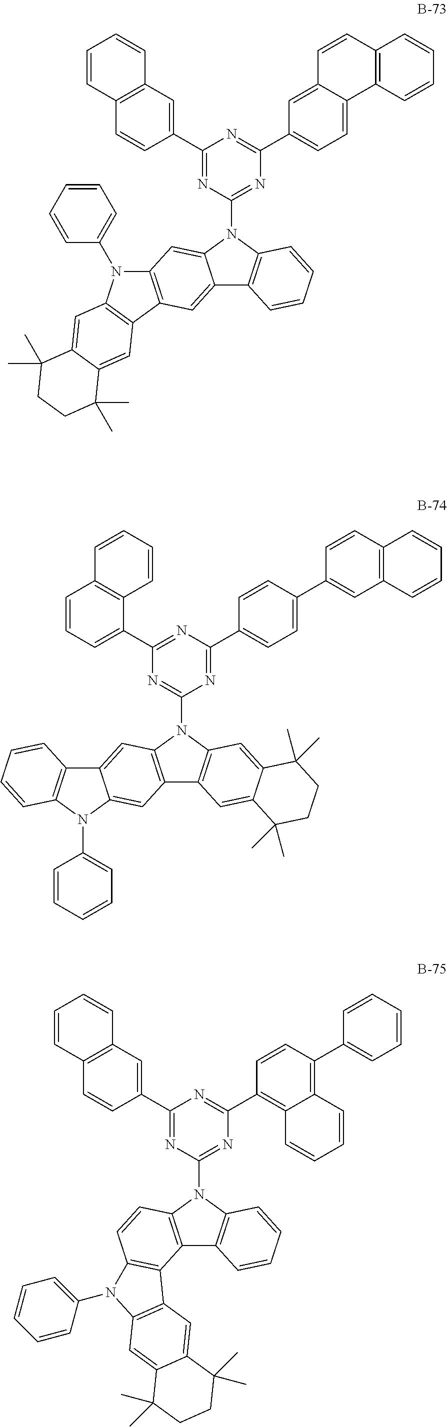 [SMILES: CC1(C)CCC(C)(C)c2cc3c(cc21)c1cc2c4ccccc4n(-c4nc(-c5ccc6ccccc6c5)nc(-c5ccc6c(ccc7ccccc76)c5)n4)c2cc1n3-c1ccccc1 ; CC1(C)CCC(C)(C)c2cc3c(cc21)c1cc2c(cc1n3-c1nc(-c3ccc(-c4ccc5ccccc5c4)cc3)nc(-c3cccc4ccccc34)n1)c1ccccc1n2-c1ccccc1 ; CC1(C)CCC(C)(C)c2cc3c(cc21)c1c2c4ccccc4n(-c4nc(-c5ccc6ccccc6c5)nc(-c5ccc(-c6ccccc6)c6ccccc56)n4)c2ccc1n3-c1ccccc1]

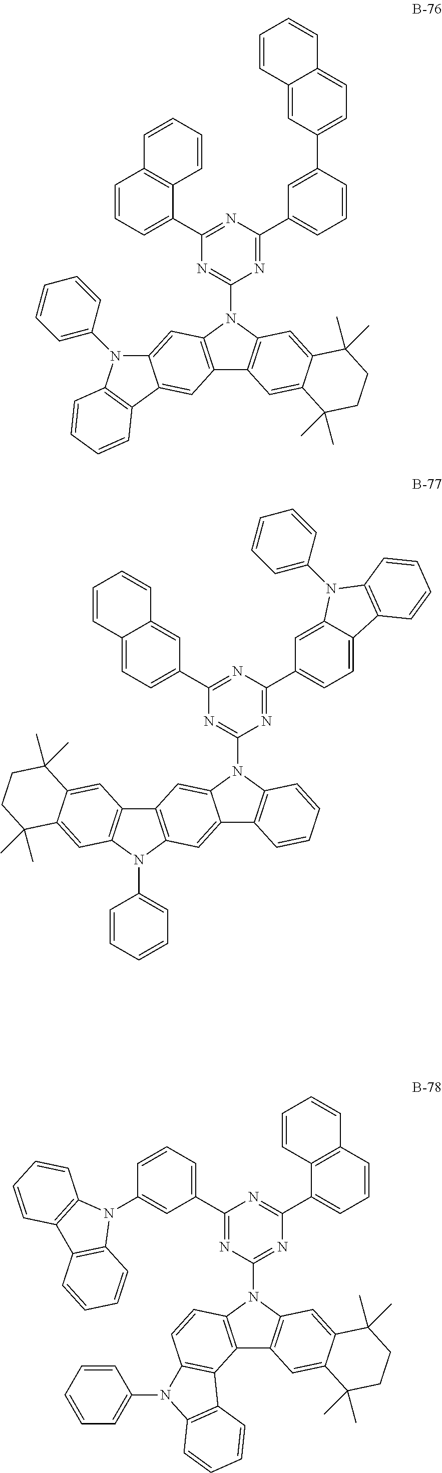

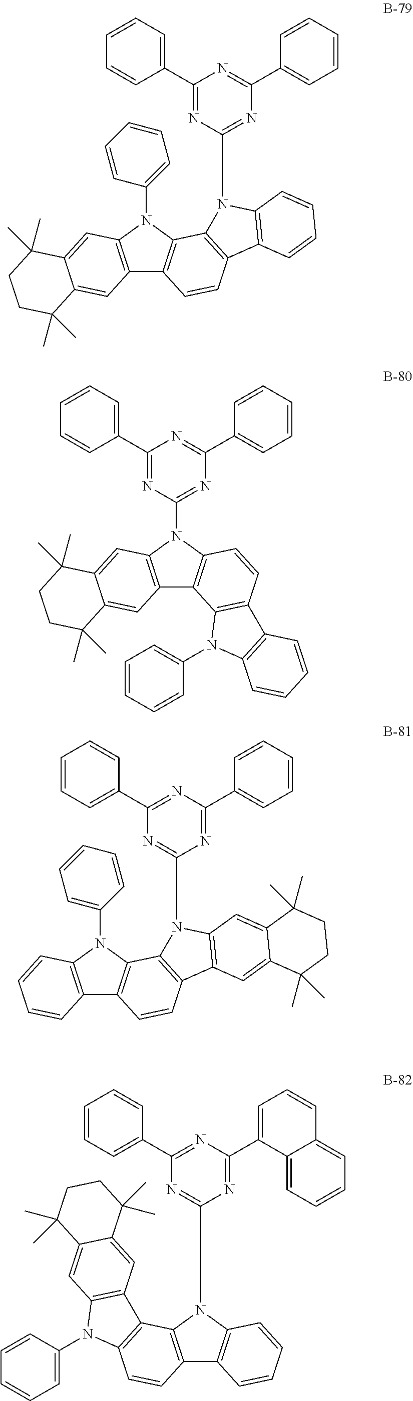 [SMILES: CC1(C)CCC(C)(C)c2cc3c(cc21)c1ccc2c4ccccc4n(-c4nc(-c5ccccc5)nc(-c5ccccc5)n4)c2c1n3-c1ccccc1 ; CC1(C)CCC(C)(C)c2cc3c(cc21)c1c(ccc2c4ccccc4n(-c4ccccc4)c21)n3-c1nc(-c2ccccc2)nc(-c2ccccc2)n1 ; CC1(C)CCC(C)(C)c2cc3c(cc21)c1ccc2c4ccccc4n(-c4ccccc4)c2c1n3-c1nc(-c2ccccc2)nc(-c2ccccc2)n1 ; CC1(C)CCC(C)(C)c2cc3c(cc21)c1c(ccc2c4ccccc4n(-c4nc(-c5ccccc5)nc(-c5cccc6ccccc56)n4)c21)n3-c1ccccc1]

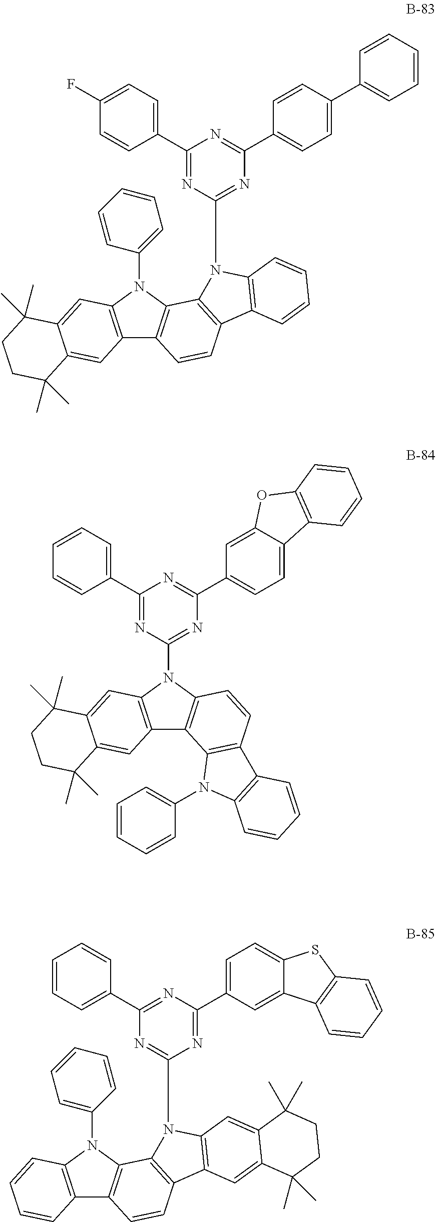 [SMILES: CC1(C)CCC(C)(C)c2cc3c(cc21)c1ccc2c4ccccc4n(-c4nc(-c5ccc(F)cc5)nc(-c5ccc(-c6ccccc6)cc5)n4)c2c1n3-c1ccccc1 ; CC1(C)CCC(C)(C)c2cc3c(cc21)c1c(ccc2c4ccccc4n(-c4ccccc4)c21)n3-c1nc(-c2ccccc2)nc(-c2ccc3c(c2)oc2ccccc23)n1 ; CC1(C)CCC(C)(C)c2cc3c(cc21)c1ccc2c4ccccc4n(-c4ccccc4)c2c1n3-c1nc(-c2ccccc2)nc(-c2ccc3sc4ccccc4c3c2)n1]

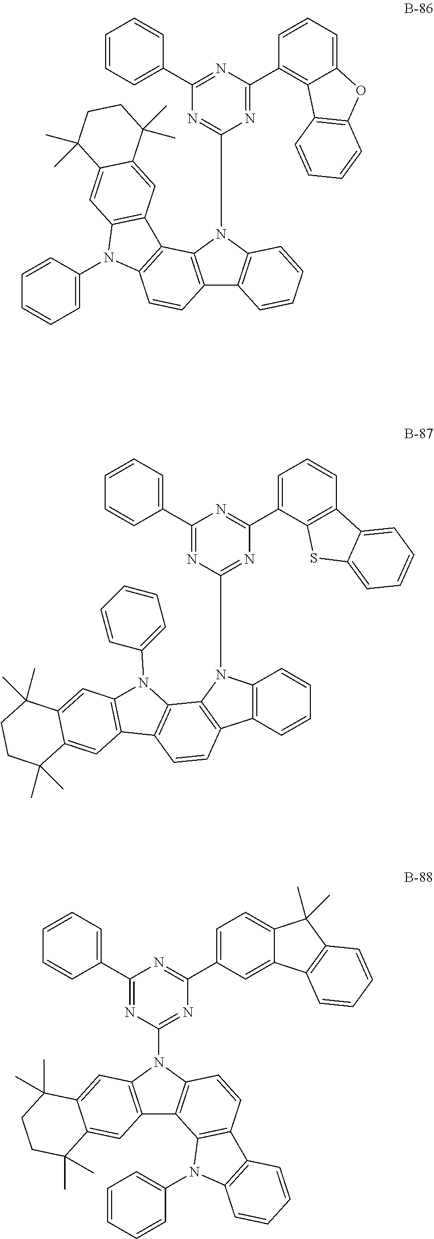 [SMILES: CC1(C)CCC(C)(C)c2cc3c(cc21)c1c(ccc2c4ccccc4n(-c4nc(-c5ccccc5)nc(-c5cccc6oc7ccccc7c56)n4)c21)n3-c1ccccc1 ; CC1(C)CCC(C)(C)c2cc3c(cc21)c1ccc2c4ccccc4n(-c4nc(-c5ccccc5)nc(-c5cccc6c5sc5ccccc56)n4)c2c1n3-c1ccccc1 ; CC1(C)CCC(C)(C)c2cc3c(cc21)c1c(ccc2c4ccccc4n(-c4ccccc4)c21)n3-c1nc(-c2ccccc2)nc(-c2ccc3c(c2)-c2ccccc2C3(C)C)n1]

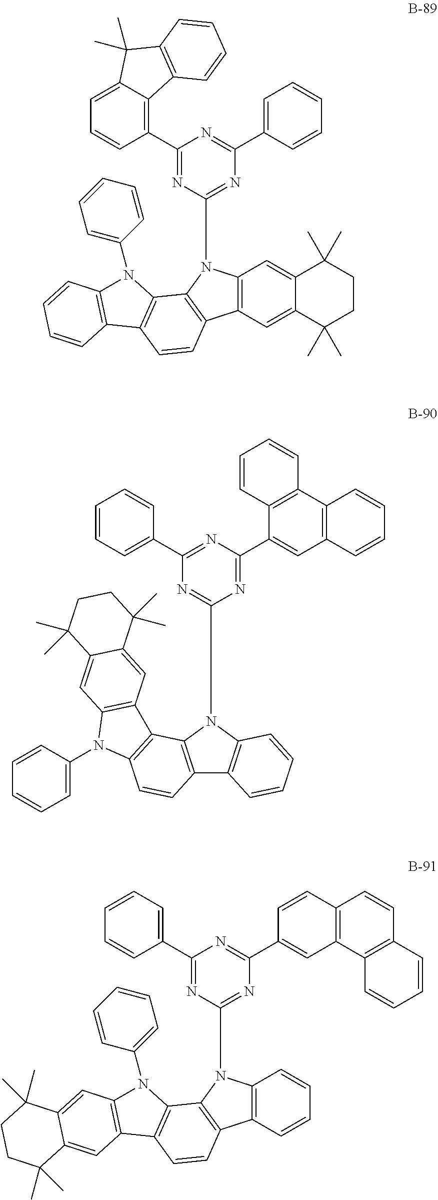 [SMILES: CC1(C)CCC(C)(C)c2cc3c(cc21)c1ccc2c4ccccc4n(-c4ccccc4)c2c1n3-c1nc(-c2ccccc2)nc(-c2cccc3c2-c2ccccc2C3(C)C)n1 ; CC1(C)CCC(C)(C)c2cc3c(cc21)c1c(ccc2c4ccccc4n(-c4nc(-c5ccccc5)nc(-c5cc6ccccc6c6ccccc56)n4)c21)n3-c1ccccc1 ; CC1(C)CCC(C)(C)c2cc3c(cc21)c1ccc2c4ccccc4n(-c4nc(-c5ccccc5)nc(-c5ccc6ccc7ccccc7c6c5)n4)c2c1n3-c1ccccc1]

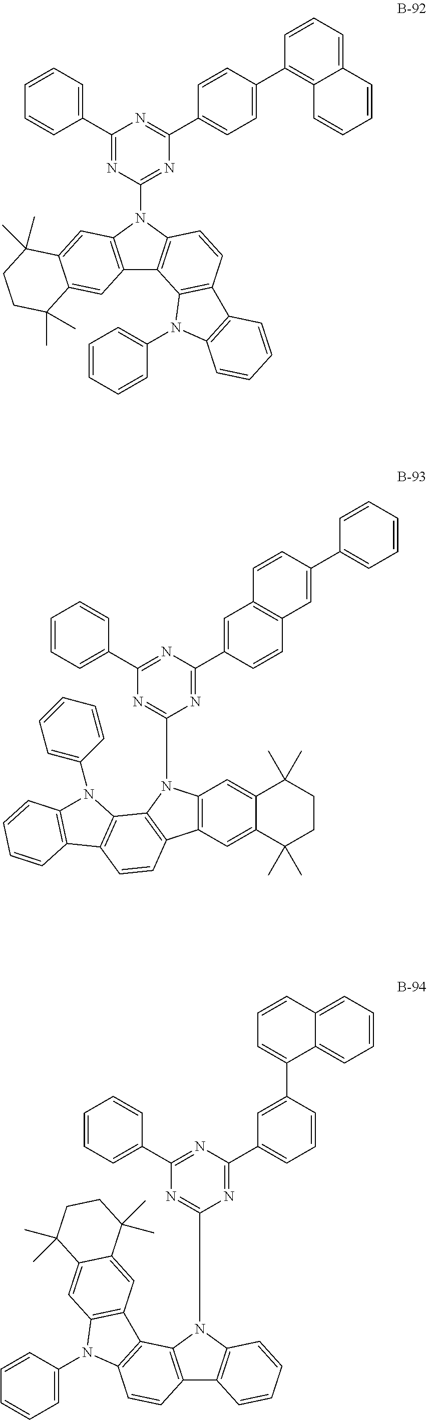 [SMILES: CC1(C)CCC(C)(C)c2cc3c(cc21)c1c(ccc2c4ccccc4n(-c4ccccc4)c21)n3-c1nc(-c2ccccc2)nc(-c2ccc(-c3cccc4ccccc34)cc2)n1 ; CC1(C)CCC(C)(C)c2cc3c(cc21)c1ccc2c4ccccc4n(-c4ccccc4)c2c1n3-c1nc(-c2ccccc2)nc(-c2ccc3cc(-c4ccccc4)ccc3c2)n1 ; CC1(C)CCC(C)(C)c2cc3c(cc21)c1c(ccc2c4ccccc4n(-c4nc(-c5ccccc5)nc(-c5cccc(-c6cccc7ccccc67)c5)n4)c21)n3-c1ccccc1]

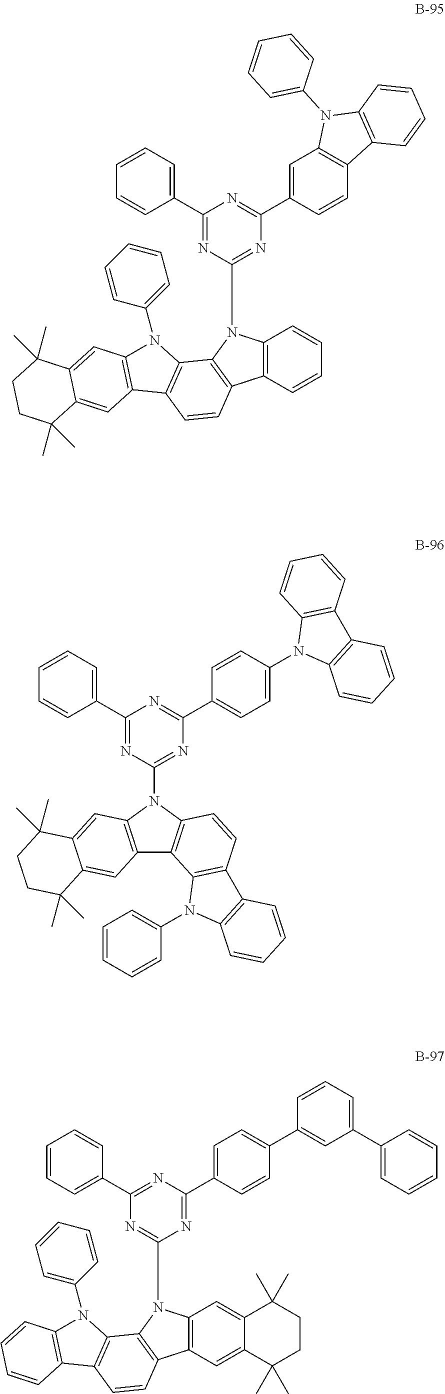 [SMILES: CC1(C)CCC(C)(C)c2cc3c(cc21)c1ccc2c4ccccc4n(-c4nc(-c5ccccc5)nc(-c5ccc6c7ccccc7n(-c7ccccc7)c6c5)n4)c2c1n3-c1ccccc1 ; CC1(C)CCC(C)(C)c2cc3c(cc21)c1c(ccc2c4ccccc4n(-c4ccccc4)c21)n3-c1nc(-c2ccccc2)nc(-c2ccc(-n3c4ccccc4c4ccccc43)cc2)n1 ; CC1(C)CCC(C)(C)c2cc3c(cc21)c1ccc2c4ccccc4n(-c4ccccc4)c2c1n3-c1nc(-c2ccccc2)nc(-c2ccc(-c3cccc(-c4ccccc4)c3)cc2)n1]

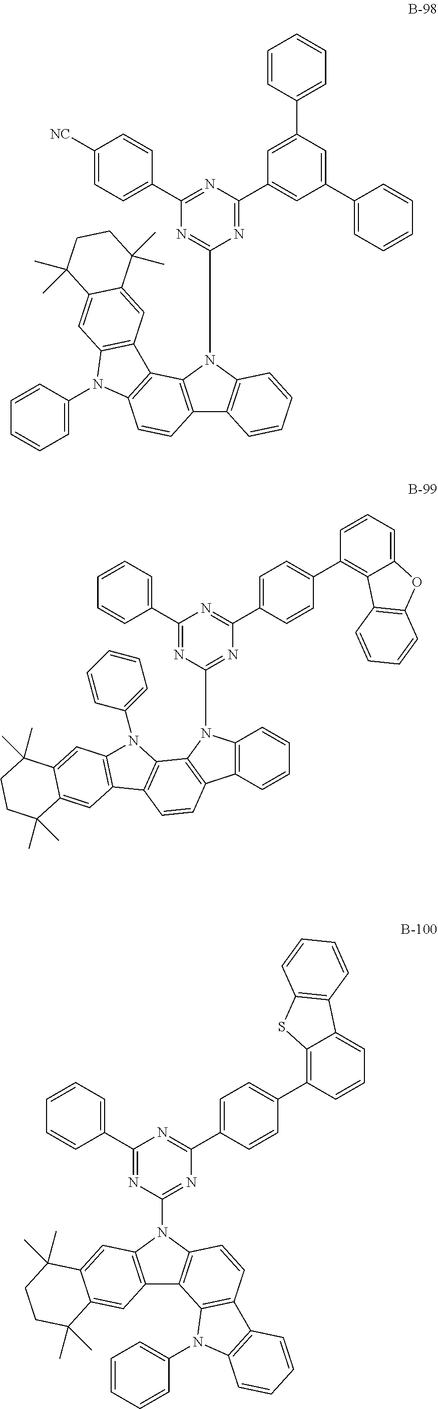 [SMILES: CC1(C)CCC(C)(C)c2cc3c(cc21)c1c(ccc2c4ccccc4n(-c4nc(-c5ccc(C#N)cc5)nc(-c5cc(-c6ccccc6)cc(-c6ccccc6)c5)n4)c21)n3-c1ccccc1 ; CC1(C)CCC(C)(C)c2cc3c(cc21)c1ccc2c4ccccc4n(-c4nc(-c5ccccc5)nc(-c5ccc(-c6cccc7oc8ccccc8c67)cc5)n4)c2c1n3-c1ccccc1 ; CC1(C)CCC(C)(C)c2cc3c(cc21)c1c(ccc2c4ccccc4n(-c4ccccc4)c21)n3-c1nc(-c2ccccc2)nc(-c2ccc(-c3cccc4c3sc3ccccc34)cc2)n1]

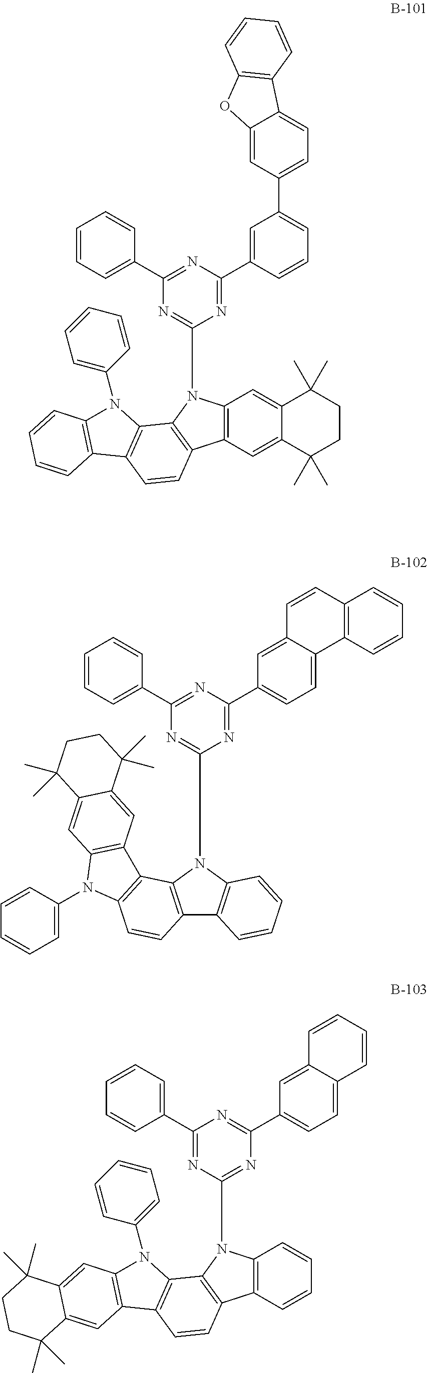 [SMILES: CC1(C)CCC(C)(C)c2cc3c(cc21)c1ccc2c4ccccc4n(-c4ccccc4)c2c1n3-c1nc(-c2ccccc2)nc(-c2cccc(-c3ccc4c(c3)oc3ccccc34)c2)n1 ; CC1(C)CCC(C)(C)c2cc3c(cc21)c1c(ccc2c4ccccc4n(-c4nc(-c5ccccc5)nc(-c5ccc6c(ccc7ccccc76)c5)n4)c21)n3-c1ccccc1 ; CC1(C)CCC(C)(C)c2cc3c(cc21)c1ccc2c4ccccc4n(-c4nc(-c5ccccc5)nc(-c5ccc6ccccc6c5)n4)c2c1n3-c1ccccc1]

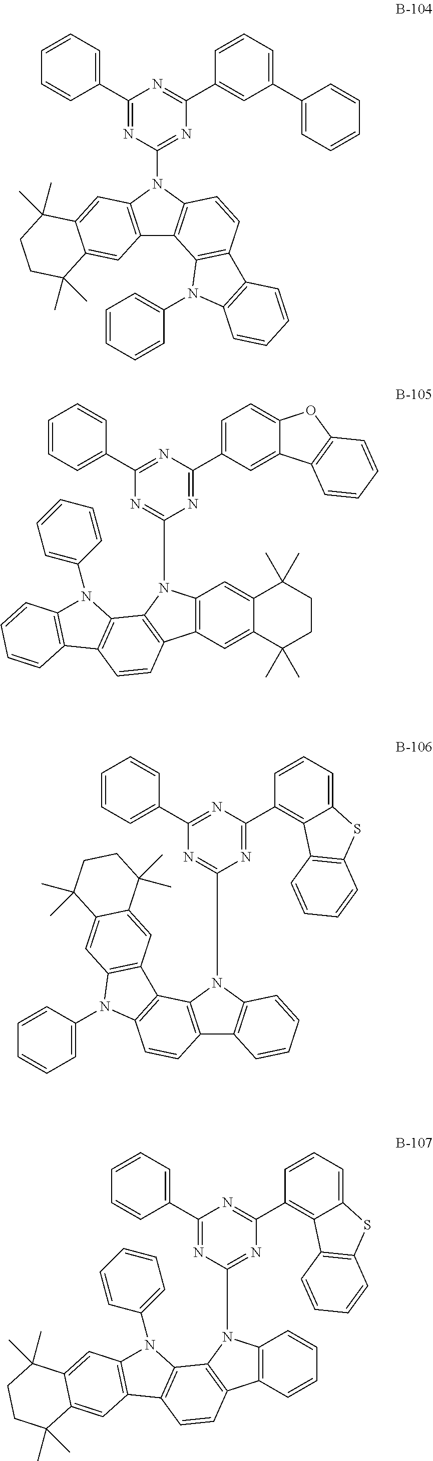 [SMILES: CC1(C)CCC(C)(C)c2cc3c(cc21)c1c(ccc2c4ccccc4n(-c4ccccc4)c21)n3-c1nc(-c2ccccc2)nc(-c2cccc(-c3ccccc3)c2)n1 ; CC1(C)CCC(C)(C)c2cc3c(cc21)c1ccc2c4ccccc4n(-c4ccccc4)c2c1n3-c1nc(-c2ccccc2)nc(-c2ccc3oc4ccccc4c3c2)n1 ; CC1(C)CCC(C)(C)c2cc3c(cc21)c1c(ccc2c4ccccc4n(-c4nc(-c5ccccc5)nc(-c5cccc6sc7ccccc7c56)n4)c21)n3-c1ccccc1 ; CC1(C)CCC(C)(C)c2cc3c(cc21)c1ccc2c4ccccc4n(-c4nc(-c5ccccc5)nc(-c5cccc6sc7ccccc7c56)n4)c2c1n3-c1ccccc1]

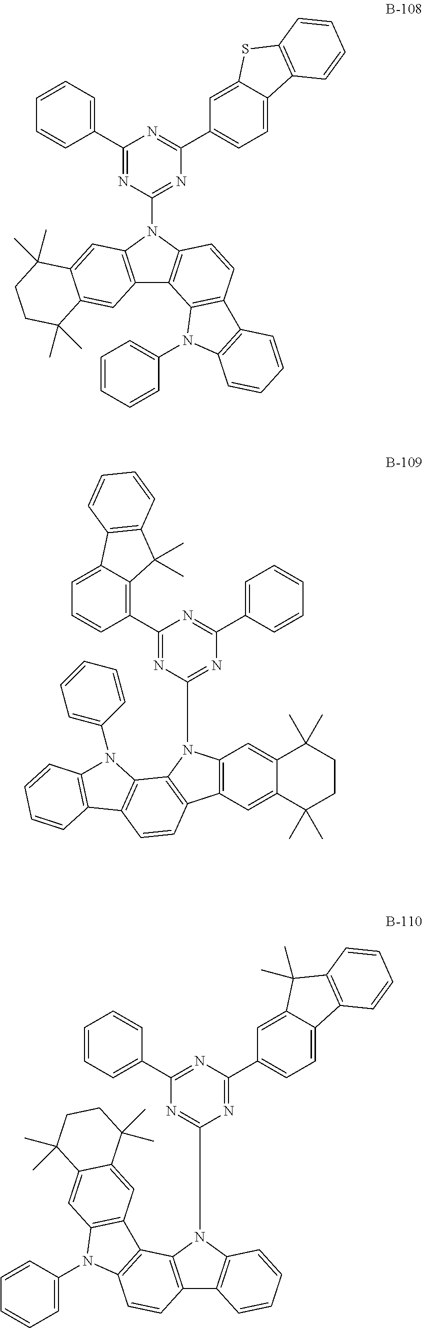 [SMILES: CC1(C)CCC(C)(C)c2cc3c(cc21)c1c(ccc2c4ccccc4n(-c4ccccc4)c21)n3-c1nc(-c2ccccc2)nc(-c2ccc3c(c2)sc2ccccc23)n1 ; CC1(C)CCC(C)(C)c2cc3c(cc21)c1ccc2c4ccccc4n(-c4ccccc4)c2c1n3-c1nc(-c2ccccc2)nc(-c2cccc3c2C(C)(C)c2ccccc2-3)n1 ; CC1(C)CCC(C)(C)c2cc3c(cc21)c1c(ccc2c4ccccc4n(-c4nc(-c5ccccc5)nc(-c5ccc6c(c5)C(C)(C)c5ccccc5-6)n4)c21)n3-c1ccccc1]

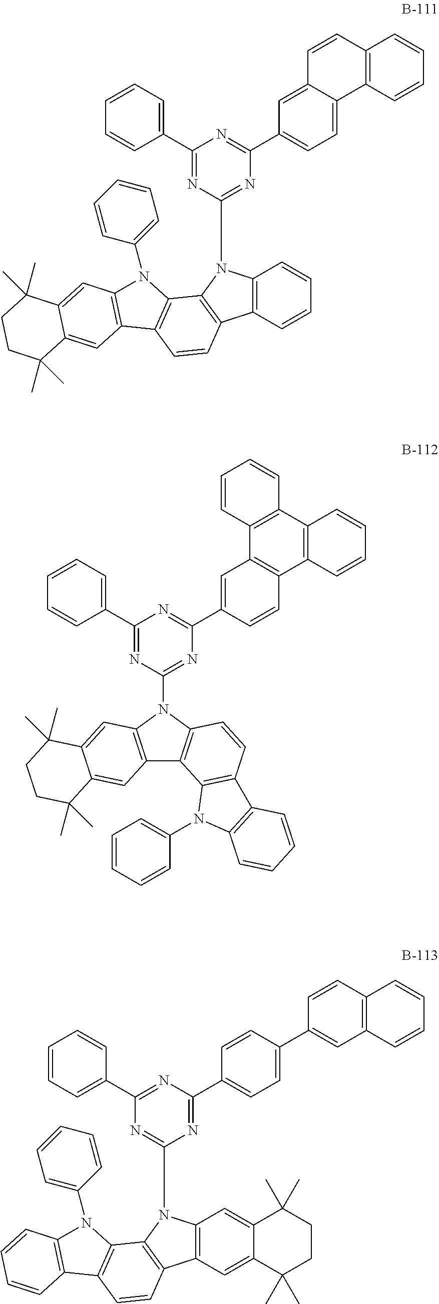 [SMILES: CC1(C)CCC(C)(C)c2cc3c(cc21)c1ccc2c4ccccc4n(-c4nc(-c5ccccc5)nc(-c5ccc6c(ccc7ccccc76)c5)n4)c2c1n3-c1ccccc1 ; CC1(C)CCC(C)(C)c2cc3c(cc21)c1c(ccc2c4ccccc4n(-c4ccccc4)c21)n3-c1nc(-c2ccccc2)nc(-c2ccc3c4ccccc4c4ccccc4c3c2)n1 ; CC1(C)CCC(C)(C)c2cc3c(cc21)c1ccc2c4ccccc4n(-c4ccccc4)c2c1n3-c1nc(-c2ccccc2)nc(-c2ccc(-c3ccc4ccccc4c3)cc2)n1]

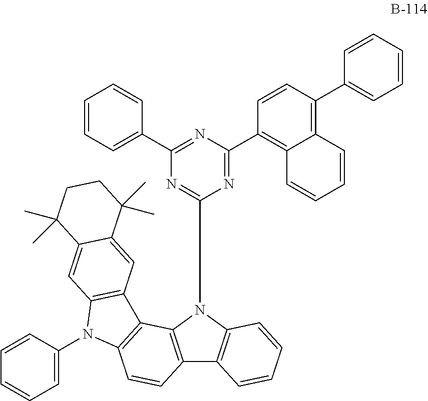 [SMILES: CC1(C)CCC(C)(C)c2cc3c(cc21)c1c(ccc2c4ccccc4n(-c4nc(-c5ccccc5)nc(-c5ccc(-c6ccccc6)c6ccccc56)n4)c21)n3-c1ccccc1]

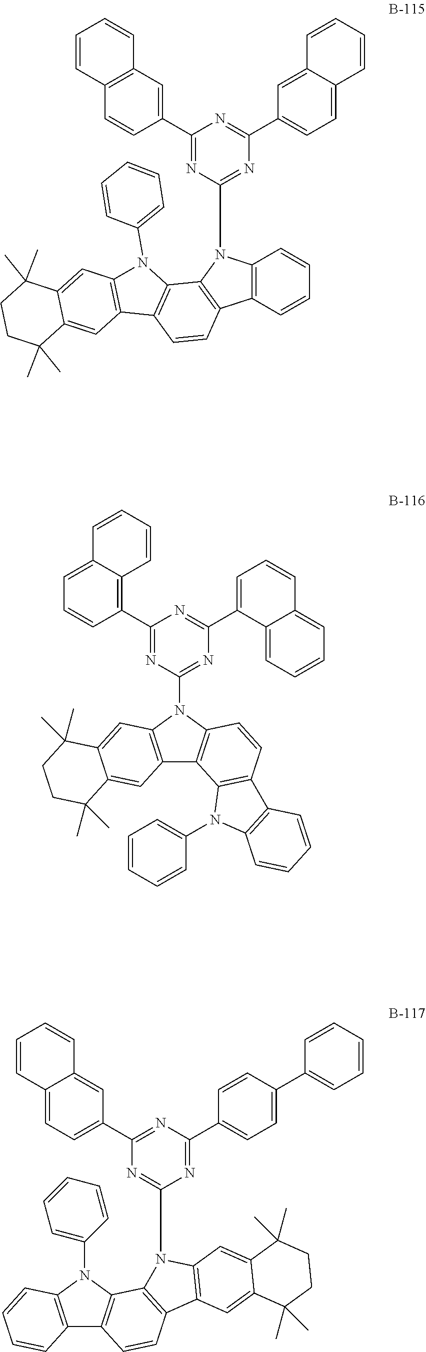 [SMILES: CC1(C)CCC(C)(C)c2cc3c(cc21)c1ccc2c4ccccc4n(-c4nc(-c5ccc6ccccc6c5)nc(-c5ccc6ccccc6c5)n4)c2c1n3-c1ccccc1 ; CC1(C)CCC(C)(C)c2cc3c(cc21)c1c(ccc2c4ccccc4n(-c4ccccc4)c21)n3-c1nc(-c2cccc3ccccc23)nc(-c2cccc3ccccc23)n1 ; CC1(C)CCC(C)(C)c2cc3c(cc21)c1ccc2c4ccccc4n(-c4ccccc4)c2c1n3-c1nc(-c2ccc(-c3ccccc3)cc2)nc(-c2ccc3ccccc3c2)n1]

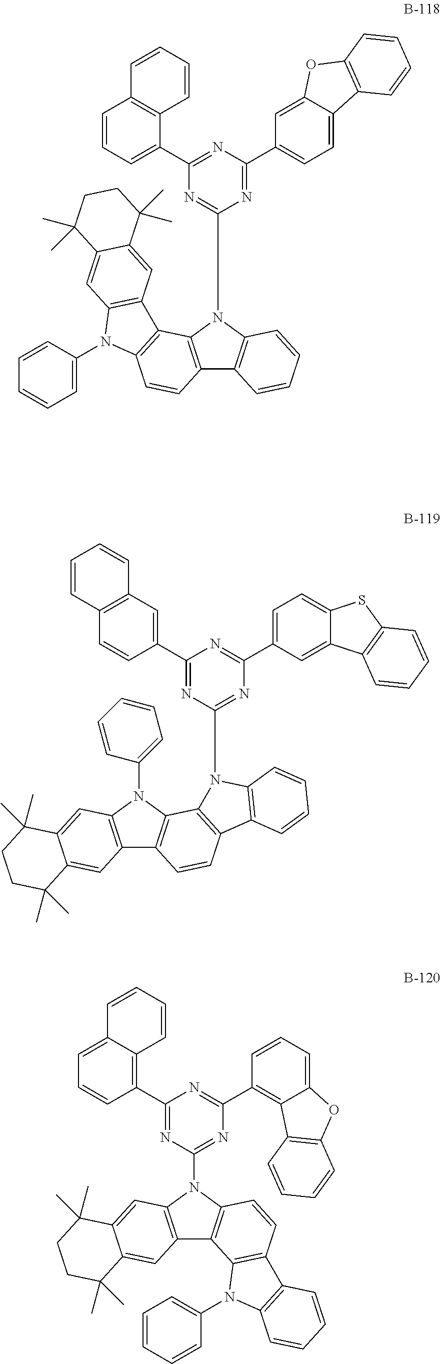 [SMILES: CC1(C)CCC(C)(C)c2cc3c(cc21)c1c(ccc2c4ccccc4n(-c4nc(-c5ccc6c(c5)oc5ccccc56)nc(-c5cccc6ccccc56)n4)c21)n3-c1ccccc1 ; CC1(C)CCC(C)(C)c2cc3c(cc21)c1ccc2c4ccccc4n(-c4nc(-c5ccc6ccccc6c5)nc(-c5ccc6sc7ccccc7c6c5)n4)c2c1n3-c1ccccc1 ; CC1(C)CCC(C)(C)c2cc3c(cc21)c1c(ccc2c4ccccc4n(-c4ccccc4)c21)n3-c1nc(-c2cccc3ccccc23)nc(-c2cccc3oc4ccccc4c23)n1]

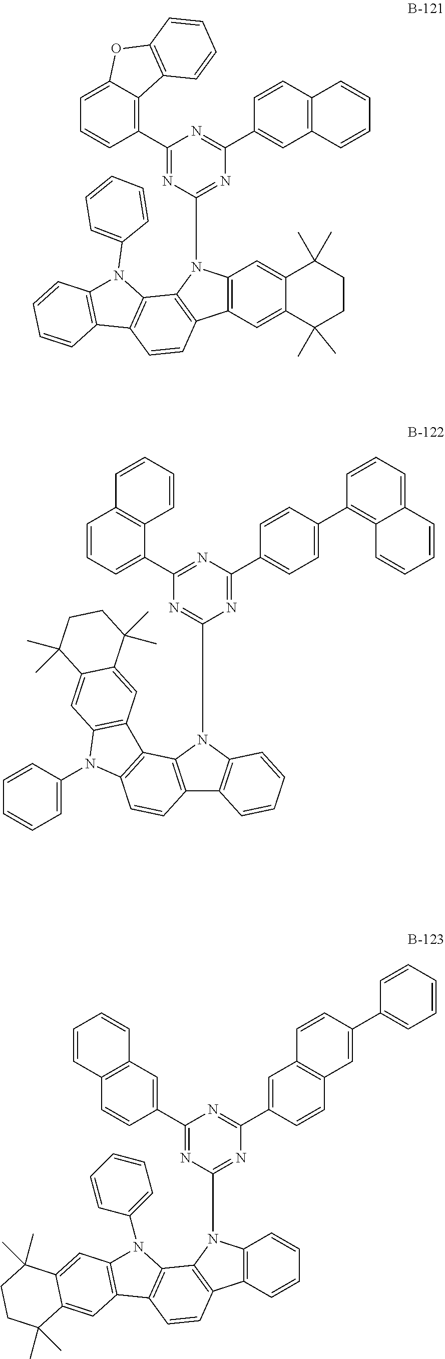 [SMILES: CC1(C)CCC(C)(C)c2cc3c(cc21)c1ccc2c4ccccc4n(-c4ccccc4)c2c1n3-c1nc(-c2ccc3ccccc3c2)nc(-c2cccc3oc4ccccc4c23)n1 ; CC1(C)CCC(C)(C)c2cc3c(cc21)c1c(ccc2c4ccccc4n(-c4nc(-c5ccc(-c6cccc7ccccc67)cc5)nc(-c5cccc6ccccc56)n4)c21)n3-c1ccccc1 ; CC1(C)CCC(C)(C)c2cc3c(cc21)c1ccc2c4ccccc4n(-c4nc(-c5ccc6ccccc6c5)nc(-c5ccc6cc(-c7ccccc7)ccc6c5)n4)c2c1n3-c1ccccc1]

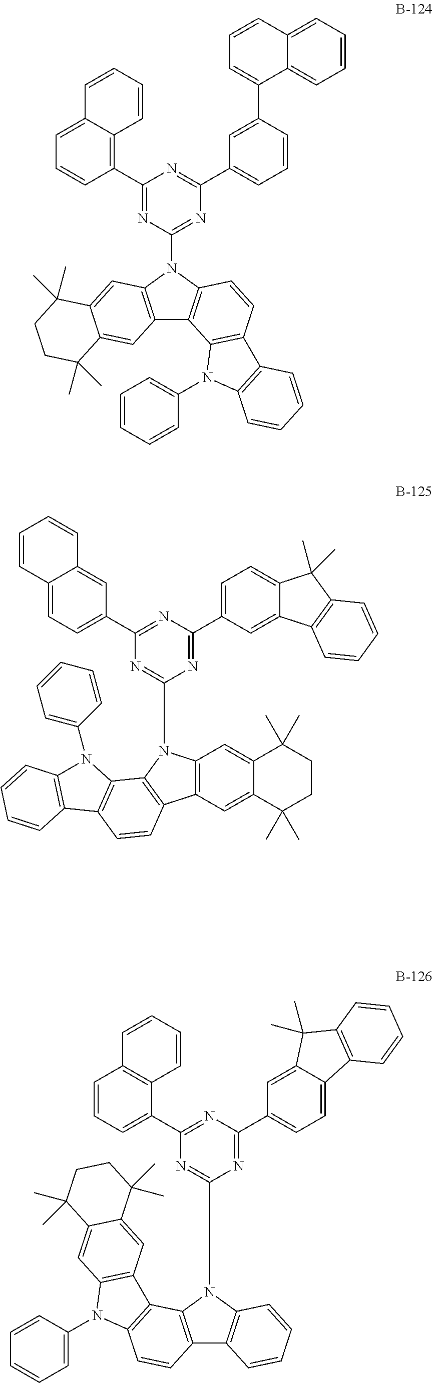 [SMILES: CC1(C)CCC(C)(C)c2cc3c(cc21)c1c(ccc2c4ccccc4n(-c4ccccc4)c21)n3-c1nc(-c2cccc(-c3cccc4ccccc34)c2)nc(-c2cccc3ccccc23)n1 ; CC1(C)CCC(C)(C)c2cc3c(cc21)c1ccc2c4ccccc4n(-c4ccccc4)c2c1n3-c1nc(-c2ccc3c(c2)-c2ccccc2C3(C)C)nc(-c2ccc3ccccc3c2)n1 ; CC1(C)CCC(C)(C)c2cc3c(cc21)c1c(ccc2c4ccccc4n(-c4nc(-c5ccc6c(c5)C(C)(C)c5ccccc5-6)nc(-c5cccc6ccccc56)n4)c21)n3-c1ccccc1]

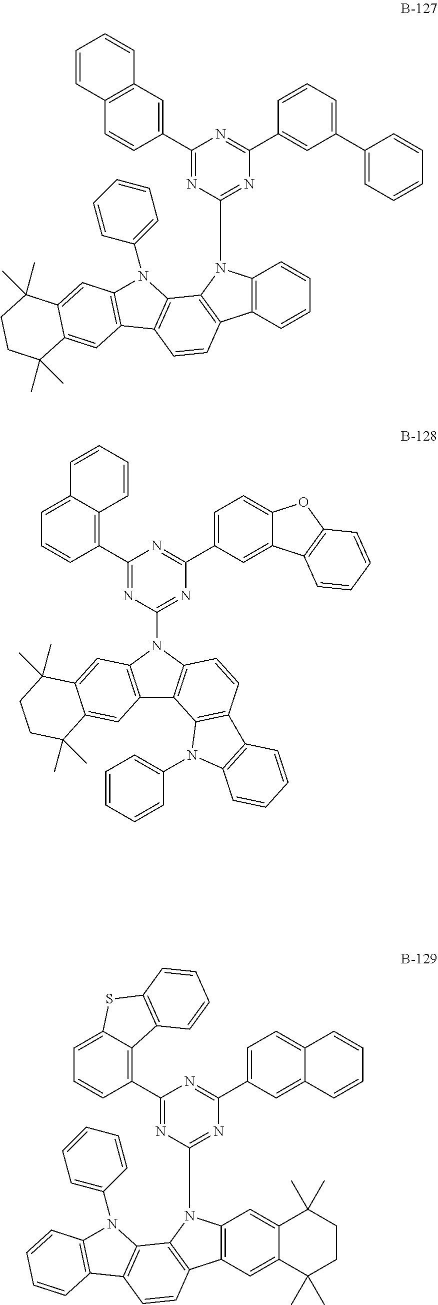 [SMILES: CC1(C)CCC(C)(C)c2cc3c(cc21)c1ccc2c4ccccc4n(-c4nc(-c5cccc(-c6ccccc6)c5)nc(-c5ccc6ccccc6c5)n4)c2c1n3-c1ccccc1 ; CC1(C)CCC(C)(C)c2cc3c(cc21)c1c(ccc2c4ccccc4n(-c4ccccc4)c21)n3-c1nc(-c2ccc3oc4ccccc4c3c2)nc(-c2cccc3ccccc23)n1 ; CC1(C)CCC(C)(C)c2cc3c(cc21)c1ccc2c4ccccc4n(-c4ccccc4)c2c1n3-c1nc(-c2ccc3ccccc3c2)nc(-c2cccc3sc4ccccc4c23)n1]

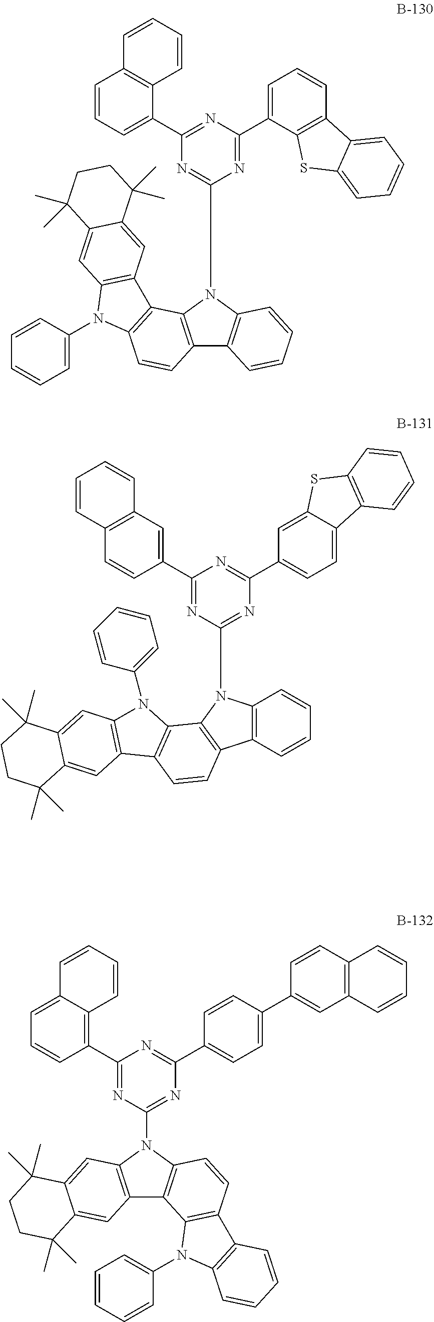 [SMILES: CC1(C)CCC(C)(C)c2cc3c(cc21)c1c(ccc2c4ccccc4n(-c4nc(-c5cccc6ccccc56)nc(-c5cccc6c5sc5ccccc56)n4)c21)n3-c1ccccc1 ; CC1(C)CCC(C)(C)c2cc3c(cc21)c1ccc2c4ccccc4n(-c4nc(-c5ccc6ccccc6c5)nc(-c5ccc6c(c5)sc5ccccc56)n4)c2c1n3-c1ccccc1 ; CC1(C)CCC(C)(C)c2cc3c(cc21)c1c(ccc2c4ccccc4n(-c4ccccc4)c21)n3-c1nc(-c2ccc(-c3ccc4ccccc4c3)cc2)nc(-c2cccc3ccccc23)n1]

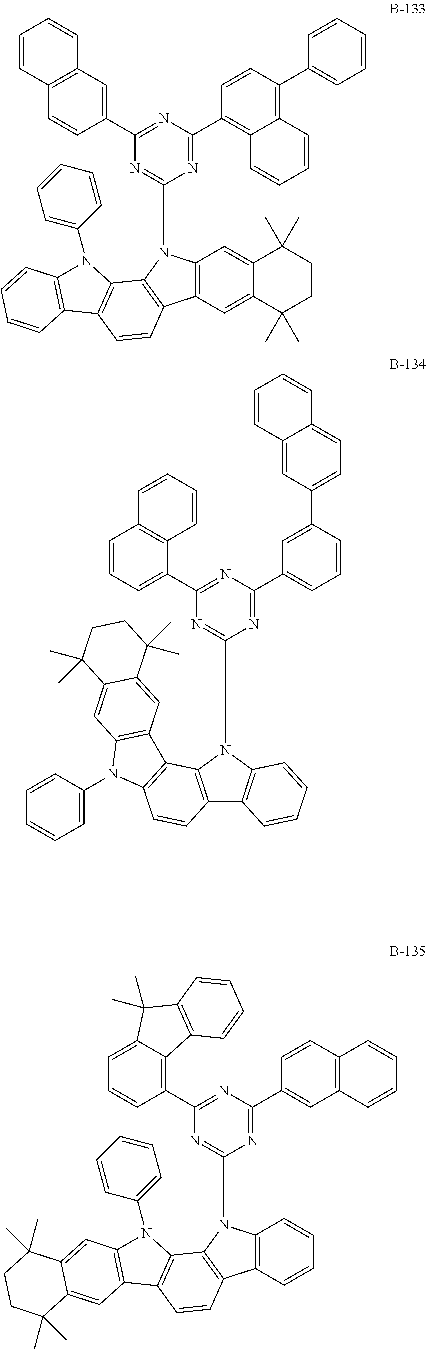

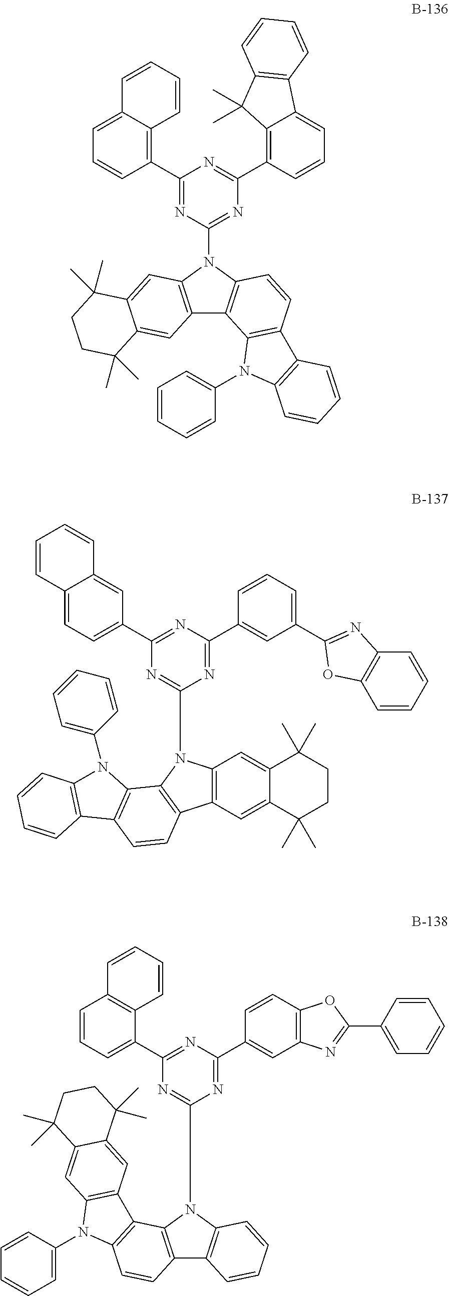 [SMILES: CC1(C)CCC(C)(C)c2cc3c(cc21)c1c(ccc2c4ccccc4n(-c4ccccc4)c21)n3-c1nc(-c2cccc3c2C(C)(C)c2ccccc2-3)nc(-c2cccc3ccccc23)n1 ; CC1(C)CCC(C)(C)c2cc3c(cc21)c1ccc2c4ccccc4n(-c4ccccc4)c2c1n3-c1nc(-c2cccc(-c3nc4ccccc4o3)c2)nc(-c2ccc3ccccc3c2)n1 ; CC1(C)CCC(C)(C)c2cc3c(cc21)c1c(ccc2c4ccccc4n(-c4nc(-c5ccc6oc(-c7ccccc7)nc6c5)nc(-c5cccc6ccccc56)n4)c21)n3-c1ccccc1]

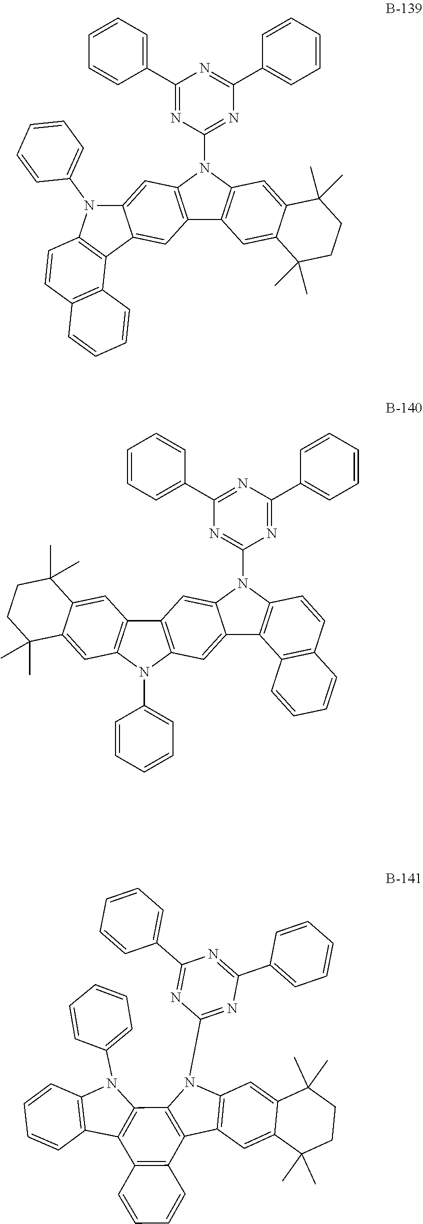

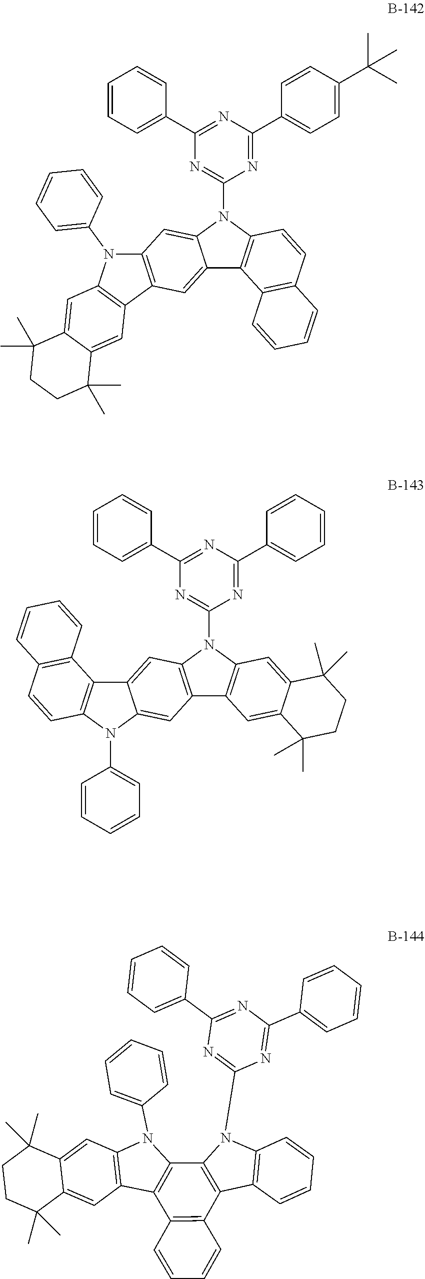 [SMILES: CC(C)(C)c1ccc(-c2nc(-c3ccccc3)nc(-n3c4cc5c(cc4c4c6ccccc6ccc43)c3cc4c(cc3n5-c3ccccc3)C(C)(C)CCC4(C)C)n2)cc1 ; CC1(C)CCC(C)(C)c2cc3c(cc21)c1cc2c(cc1n3-c1nc(-c3ccccc3)nc(-c3ccccc3)n1)c1c3ccccc3ccc1n2-c1ccccc1 ; CC1(C)CCC(C)(C)c2cc3c(cc21)c1c2ccccc2c2c4ccccc4n(-c4nc(-c5ccccc5)nc(-c5ccccc5)n4)c2c1n3-c1ccccc1]

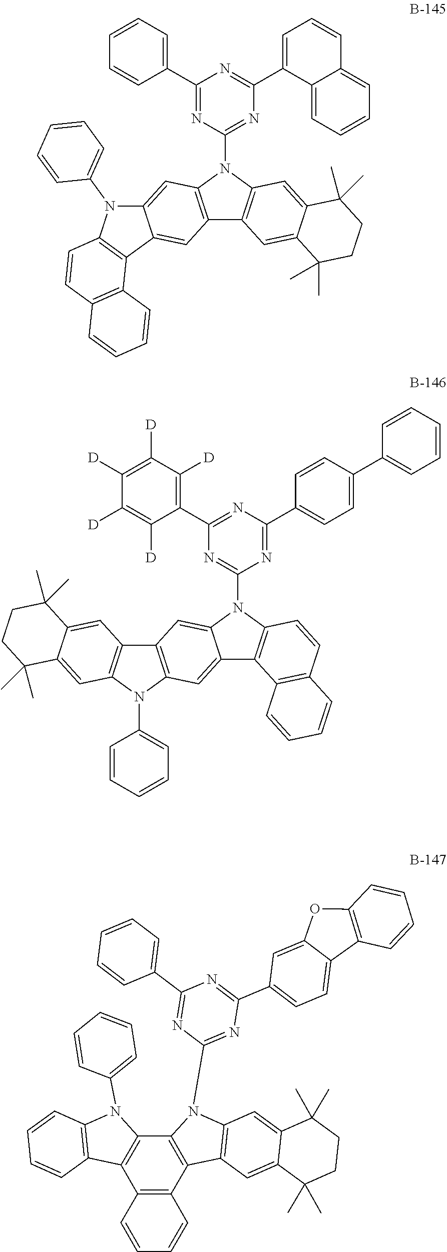 [SMILES: CC1(C)CCC(C)(C)c2cc3c(cc21)c1cc2c4c5ccccc5ccc4n(-c4ccccc4)c2cc1n3-c1nc(-c2ccccc2)nc(-c2cccc3ccccc23)n1 ; [2H]c1c([2H])c([2H])c(-c2nc(-c3ccc(-c4ccccc4)cc3)nc(-n3c4cc5c6cc7c(cc6n(-c6ccccc6)c5cc4c4c5ccccc5ccc43)C(C)(C)CCC7(C)C)n2)c([2H])c1[2H] ; CC1(C)CCC(C)(C)c2cc3c(cc21)c1c2ccccc2c2c4ccccc4n(-c4ccccc4)c2c1n3-c1nc(-c2ccccc2)nc(-c2ccc3c(c2)oc2ccccc23)n1]

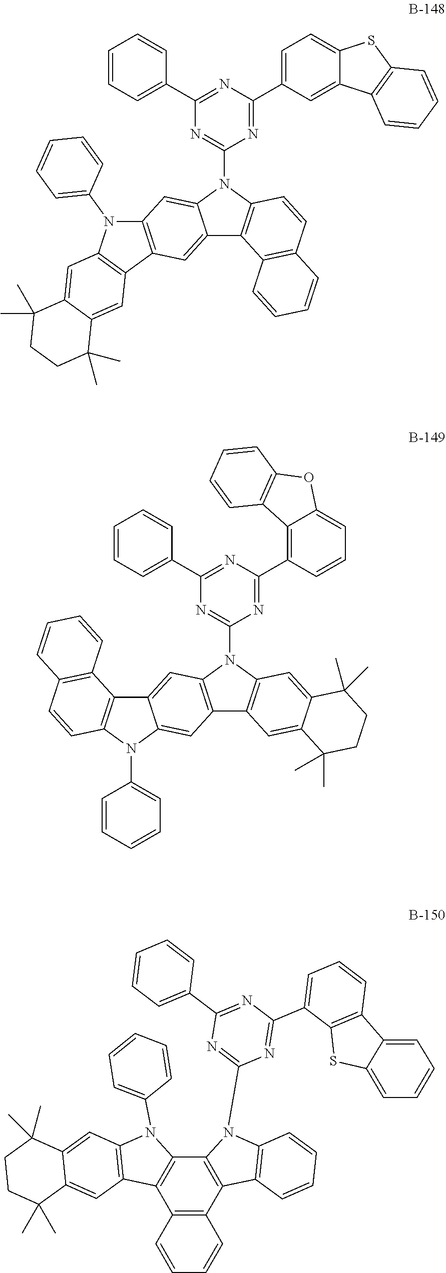 [SMILES: CC1(C)CCC(C)(C)c2cc3c(cc21)c1cc2c4c5ccccc5ccc4n(-c4nc(-c5ccccc5)nc(-c5ccc6sc7ccccc7c6c5)n4)c2cc1n3-c1ccccc1 ; CC1(C)CCC(C)(C)c2cc3c(cc21)c1cc2c(cc1n3-c1nc(-c3ccccc3)nc(-c3cccc4oc5ccccc5c34)n1)c1c3ccccc3ccc1n2-c1ccccc1 ; CC1(C)CCC(C)(C)c2cc3c(cc21)c1c2ccccc2c2c4ccccc4n(-c4nc(-c5ccccc5)nc(-c5cccc6c5sc5ccccc56)n4)c2c1n3-c1ccccc1]

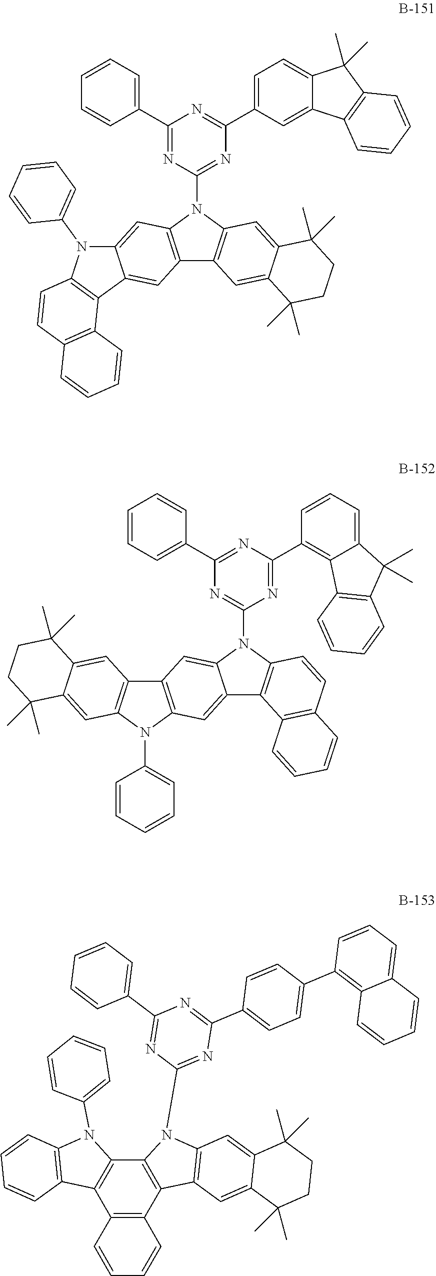

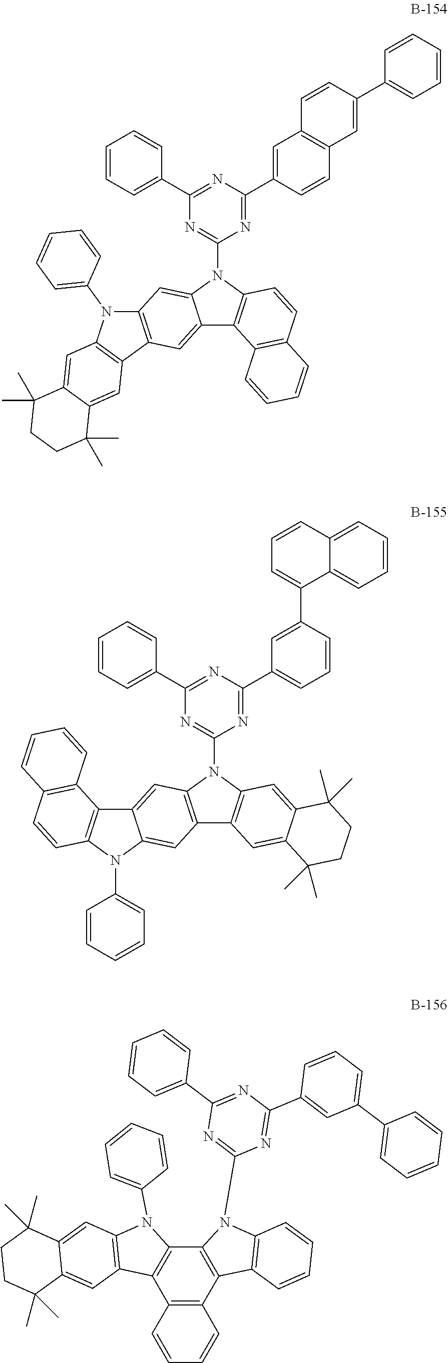 [SMILES: CC1(C)CCC(C)(C)c2cc3c(cc21)c1cc2c4c5ccccc5ccc4n(-c4nc(-c5ccccc5)nc(-c5ccc6cc(-c7ccccc7)ccc6c5)n4)c2cc1n3-c1ccccc1 ; CC1(C)CCC(C)(C)c2cc3c(cc21)c1cc2c(cc1n3-c1nc(-c3ccccc3)nc(-c3cccc(-c4cccc5ccccc45)c3)n1)c1c3ccccc3ccc1n2-c1ccccc1 ; CC1(C)CCC(C)(C)c2cc3c(cc21)c1c2ccccc2c2c4ccccc4n(-c4nc(-c5ccccc5)nc(-c5cccc(-c6ccccc6)c5)n4)c2c1n3-c1ccccc1]

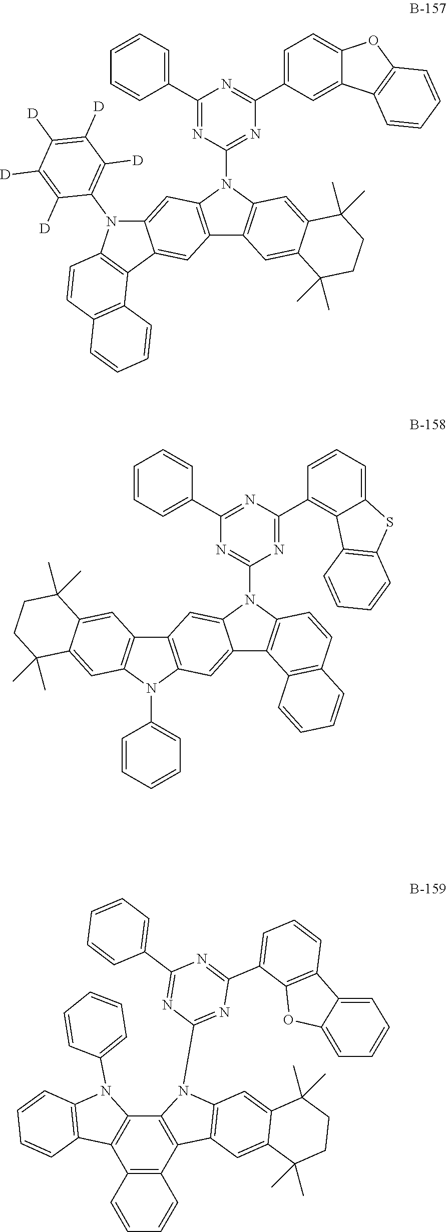 [SMILES: [2H]c1c([2H])c([2H])c(-n2c3cc4c(cc3c3c5ccccc5ccc32)c2cc3c(cc2n4-c2nc(-c4ccccc4)nc(-c4ccc5oc6ccccc6c5c4)n2)C(C)(C)CCC3(C)C)c([2H])c1[2H] ; CC1(C)CCC(C)(C)c2cc3c(cc21)c1cc2c(cc1n3-c1ccccc1)c1c3ccccc3ccc1n2-c1nc(-c2ccccc2)nc(-c2cccc3sc4ccccc4c23)n1 ; CC1(C)CCC(C)(C)c2cc3c(cc21)c1c2ccccc2c2c4ccccc4n(-c4ccccc4)c2c1n3-c1nc(-c2ccccc2)nc(-c2cccc3c2oc2ccccc23)n1]

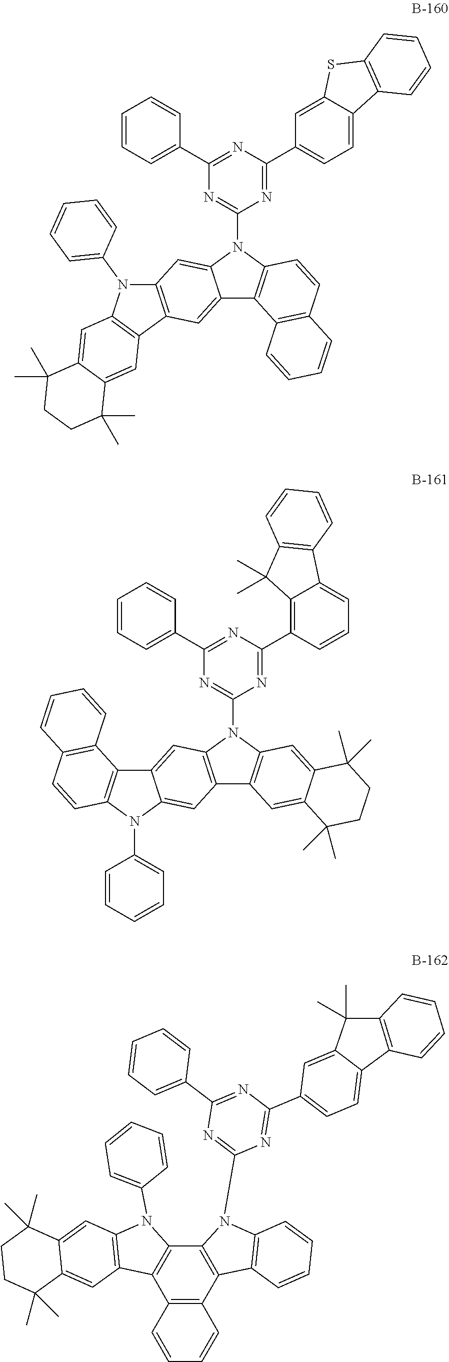 [SMILES: CC1(C)CCC(C)(C)c2cc3c(cc21)c1cc2c4c5ccccc5ccc4n(-c4nc(-c5ccccc5)nc(-c5ccc6c(c5)sc5ccccc56)n4)c2cc1n3-c1ccccc1 ; CC1(C)CCC(C)(C)c2cc3c(cc21)c1cc2c(cc1n3-c1nc(-c3ccccc3)nc(-c3cccc4c3C(C)(C)c3ccccc3-4)n1)c1c3ccccc3ccc1n2-c1ccccc1 ; CC1(C)CCC(C)(C)c2cc3c(cc21)c1c2ccccc2c2c4ccccc4n(-c4nc(-c5ccccc5)nc(-c5ccc6c(c5)C(C)(C)c5ccccc5-6)n4)c2c1n3-c1ccccc1]

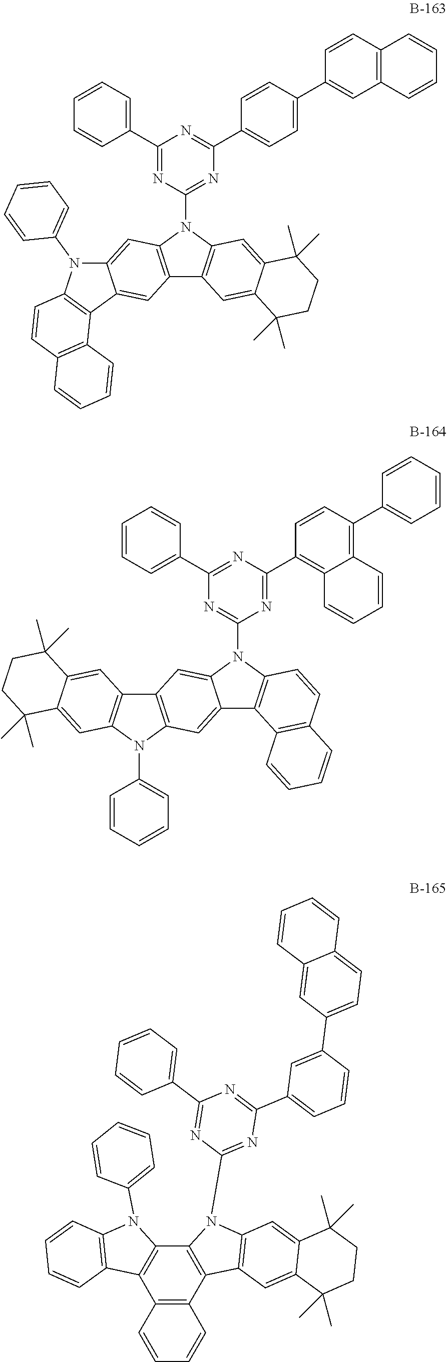 [SMILES: CC1(C)CCC(C)(C)c2cc3c(cc21)c1cc2c4c5ccccc5ccc4n(-c4ccccc4)c2cc1n3-c1nc(-c2ccccc2)nc(-c2ccc(-c3ccc4ccccc4c3)cc2)n1 ; CC1(C)CCC(C)(C)c2cc3c(cc21)c1cc2c(cc1n3-c1ccccc1)c1c3ccccc3ccc1n2-c1nc(-c2ccccc2)nc(-c2ccc(-c3ccccc3)c3ccccc23)n1 ; CC1(C)CCC(C)(C)c2cc3c(cc21)c1c2ccccc2c2c4ccccc4n(-c4ccccc4)c2c1n3-c1nc(-c2ccccc2)nc(-c2cccc(-c3ccc4ccccc4c3)c2)n1]

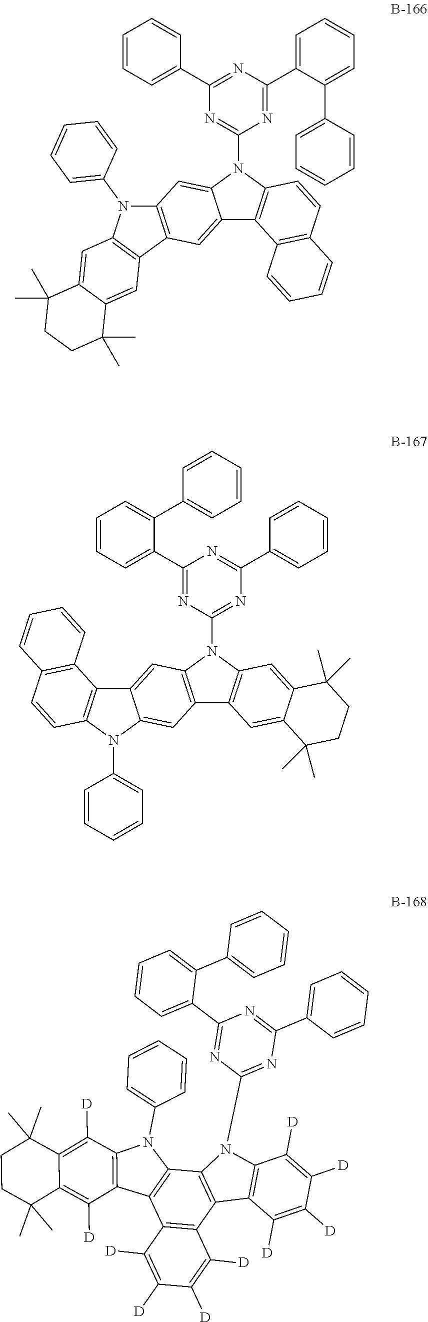 [SMILES: CC1(C)CCC(C)(C)c2cc3c(cc21)c1cc2c4c5ccccc5ccc4n(-c4nc(-c5ccccc5)nc(-c5ccccc5-c5ccccc5)n4)c2cc1n3-c1ccccc1 ; CC1(C)CCC(C)(C)c2cc3c(cc21)c1cc2c(cc1n3-c1nc(-c3ccccc3)nc(-c3ccccc3-c3ccccc3)n1)c1c3ccccc3ccc1n2-c1ccccc1 ; [2H]c1c([2H])c([2H])c2c(c1[2H])c1c3c([2H])c4c(c([2H])c3n(-c3ccccc3)c1c1c2c2c([2H])c([2H])c([2H])c([2H])c2n1-c1nc(-c2ccccc2)nc(-c2ccccc2-c2ccccc2)n1)C(C)(C)CCC4(C)C]

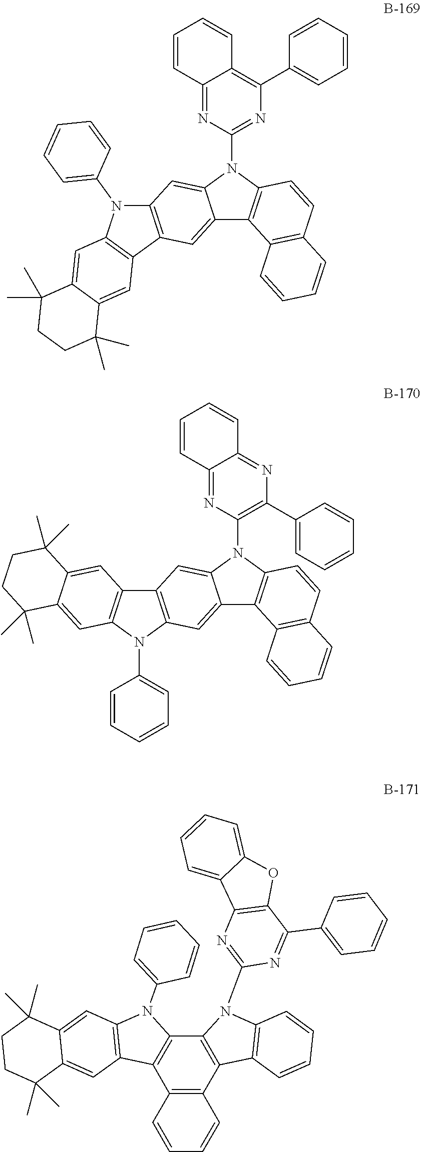 [SMILES: CC1(C)CCC(C)(C)c2cc3c(cc21)c1cc2c4c5ccccc5ccc4n(-c4nc(-c5ccccc5)c5ccccc5n4)c2cc1n3-c1ccccc1 ; CC1(C)CCC(C)(C)c2cc3c(cc21)c1cc2c(cc1n3-c1ccccc1)c1c3ccccc3ccc1n2-c1nc2ccccc2nc1-c1ccccc1 ; CC1(C)CCC(C)(C)c2cc3c(cc21)c1c2ccccc2c2c4ccccc4n(-c4nc(-c5ccccc5)c5oc6ccccc6c5n4)c2c1n3-c1ccccc1]

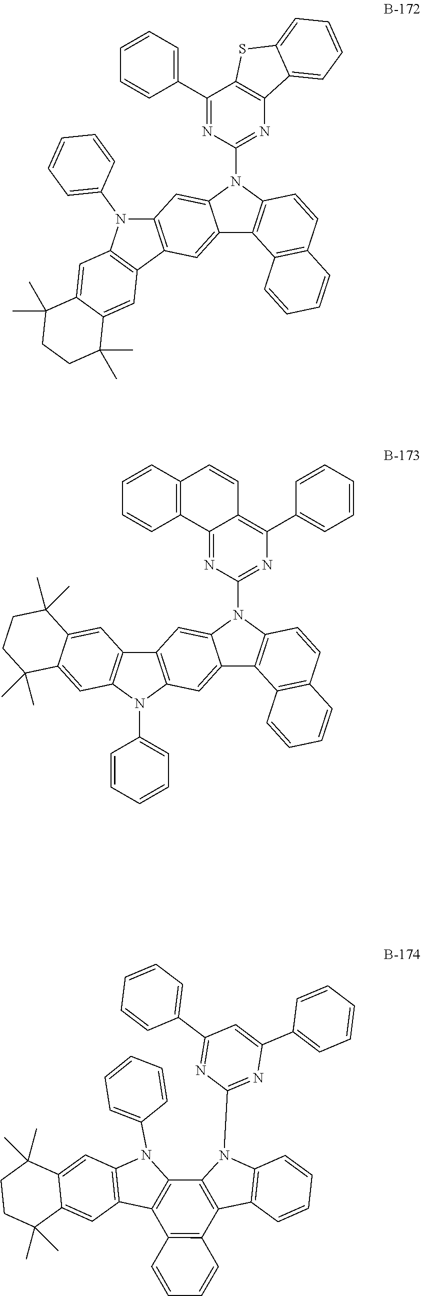 [SMILES: CC1(C)CCC(C)(C)c2cc3c(cc21)c1cc2c4c5ccccc5ccc4n(-c4nc(-c5ccccc5)c5sc6ccccc6c5n4)c2cc1n3-c1ccccc1 ; CC1(C)CCC(C)(C)c2cc3c(cc21)c1cc2c(cc1n3-c1ccccc1)c1c3ccccc3ccc1n2-c1nc(-c2ccccc2)c2ccc3ccccc3c2n1 ; CC1(C)CCC(C)(C)c2cc3c(cc21)c1c2ccccc2c2c4ccccc4n(-c4nc(-c5ccccc5)cc(-c5ccccc5)n4)c2c1n3-c1ccccc1]

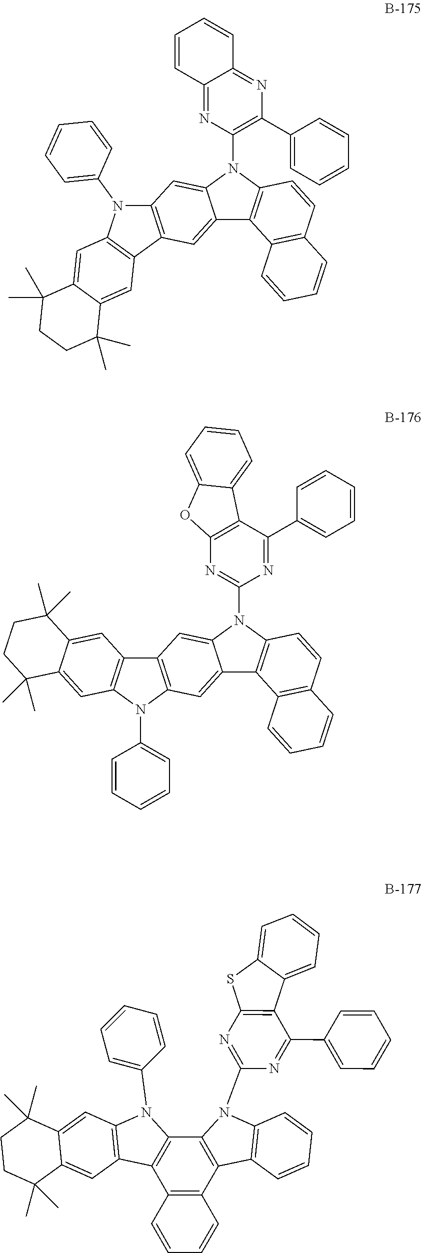 [SMILES: CC1(C)CCC(C)(C)c2cc3c(cc21)c1cc2c4c5ccccc5ccc4n(-c4nc5ccccc5nc4-c4ccccc4)c2cc1n3-c1ccccc1 ; CC1(C)CCC(C)(C)c2cc3c(cc21)c1cc2c(cc1n3-c1ccccc1)c1c3ccccc3ccc1n2-c1nc(-c2ccccc2)c2c(n1)oc1ccccc12 ; CC1(C)CCC(C)(C)c2cc3c(cc21)c1c2ccccc2c2c4ccccc4n(-c4nc(-c5ccccc5)c5c(n4)sc4ccccc45)c2c1n3-c1ccccc1]

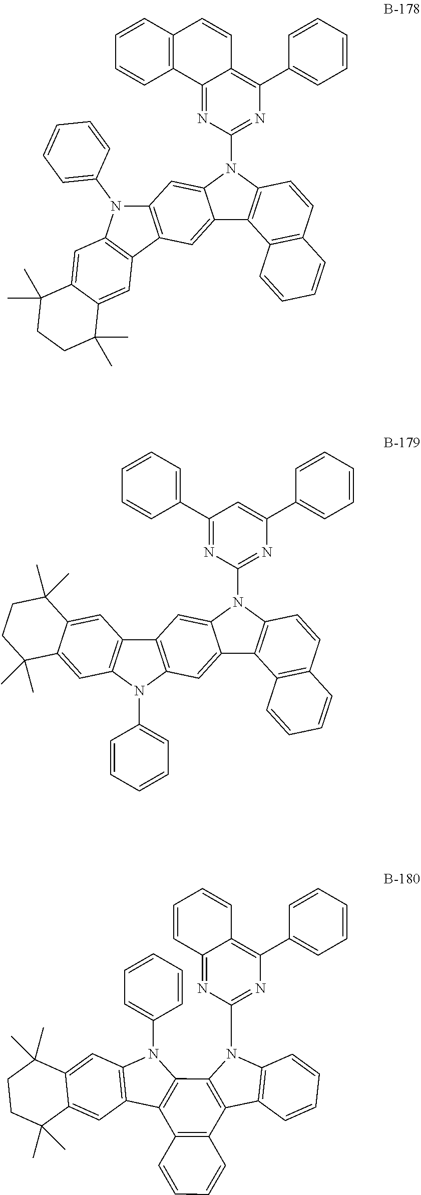 [SMILES: CC1(C)CCC(C)(C)c2cc3c(cc21)c1cc2c4c5ccccc5ccc4n(-c4nc(-c5ccccc5)c5ccc6ccccc6c5n4)c2cc1n3-c1ccccc1 ; CC1(C)CCC(C)(C)c2cc3c(cc21)c1cc2c(cc1n3-c1ccccc1)c1c3ccccc3ccc1n2-c1nc(-c2ccccc2)cc(-c2ccccc2)n1 ; CC1(C)CCC(C)(C)c2cc3c(cc21)c1c2ccccc2c2c4ccccc4n(-c4nc(-c5ccccc5)c5ccccc5n4)c2c1n3-c1ccccc1]

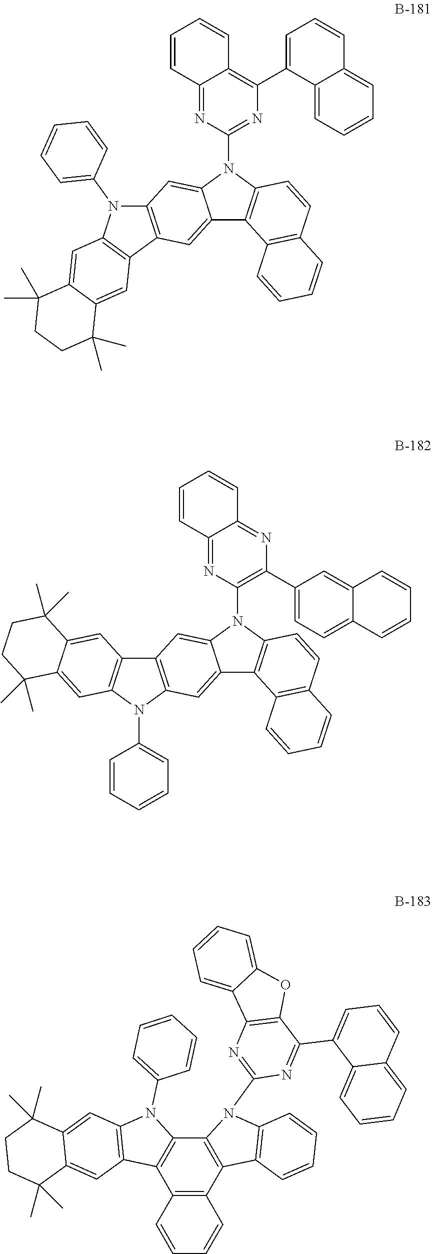 [SMILES: CC1(C)CCC(C)(C)c2cc3c(cc21)c1cc2c4c5ccccc5ccc4n(-c4nc(-c5cccc6ccccc56)c5ccccc5n4)c2cc1n3-c1ccccc1 ; CC1(C)CCC(C)(C)c2cc3c(cc21)c1cc2c(cc1n3-c1ccccc1)c1c3ccccc3ccc1n2-c1nc2ccccc2nc1-c1ccc2ccccc2c1 ; CC1(C)CCC(C)(C)c2cc3c(cc21)c1c2ccccc2c2c4ccccc4n(-c4nc(-c5cccc6ccccc56)c5oc6ccccc6c5n4)c2c1n3-c1ccccc1]

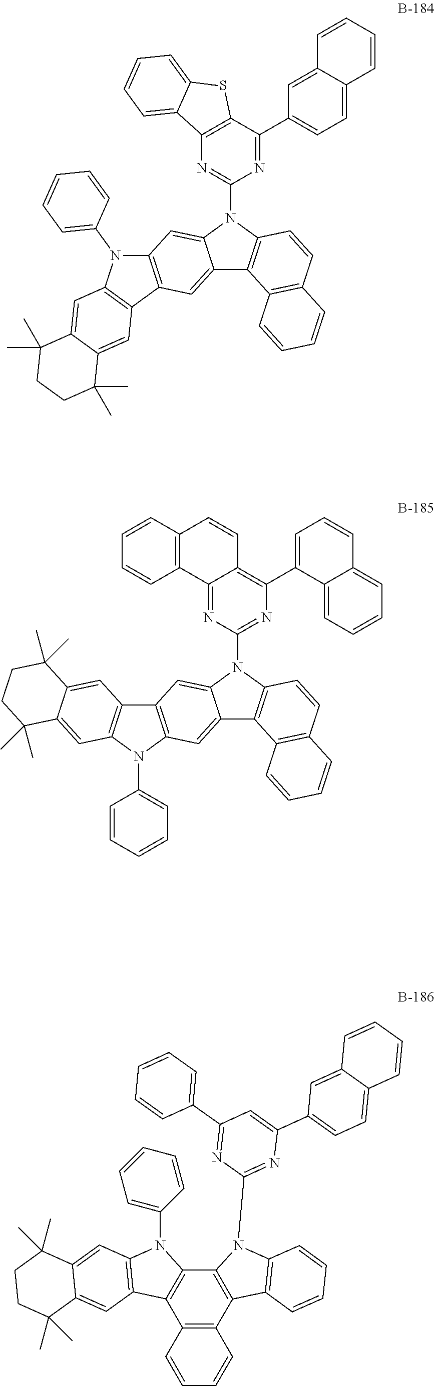 [SMILES: CC1(C)CCC(C)(C)c2cc3c(cc21)c1cc2c4c5ccccc5ccc4n(-c4nc(-c5ccc6ccccc6c5)c5sc6ccccc6c5n4)c2cc1n3-c1ccccc1 ; CC1(C)CCC(C)(C)c2cc3c(cc21)c1cc2c(cc1n3-c1ccccc1)c1c3ccccc3ccc1n2-c1nc(-c2cccc3ccccc23)c2ccc3ccccc3c2n1 ; CC1(C)CCC(C)(C)c2cc3c(cc21)c1c2ccccc2c2c4ccccc4n(-c4nc(-c5ccccc5)cc(-c5ccc6ccccc6c5)n4)c2c1n3-c1ccccc1]

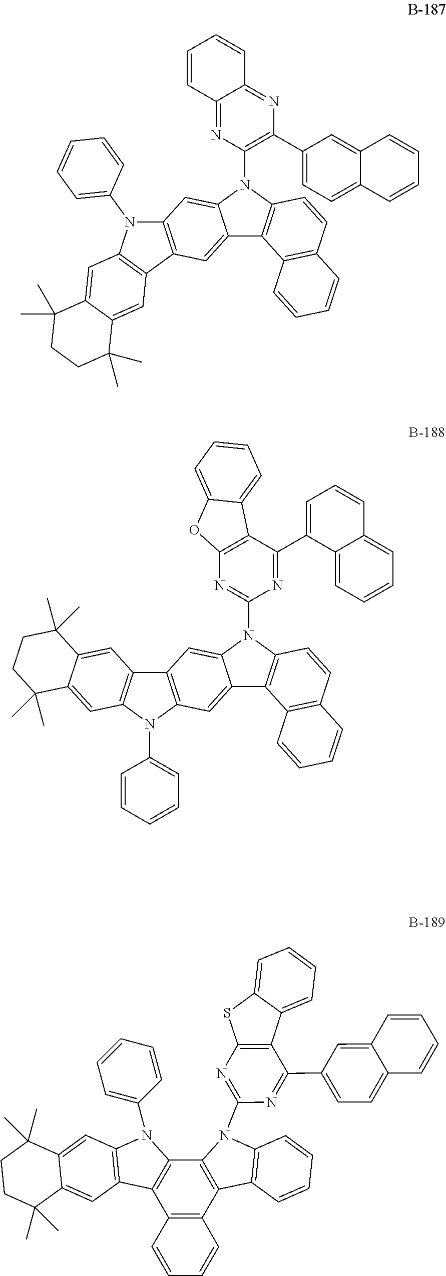 [SMILES: CC1(C)CCC(C)(C)c2cc3c(cc21)c1cc2c4c5ccccc5ccc4n(-c4nc5ccccc5nc4-c4ccc5ccccc5c4)c2cc1n3-c1ccccc1 ; CC1(C)CCC(C)(C)c2cc3c(cc21)c1cc2c(cc1n3-c1ccccc1)c1c3ccccc3ccc1n2-c1nc(-c2cccc3ccccc23)c2c(n1)oc1ccccc12 ; CC1(C)CCC(C)(C)c2cc3c(cc21)c1c2ccccc2c2c4ccccc4n(-c4nc(-c5ccc6ccccc6c5)c5c(n4)sc4ccccc45)c2c1n3-c1ccccc1]

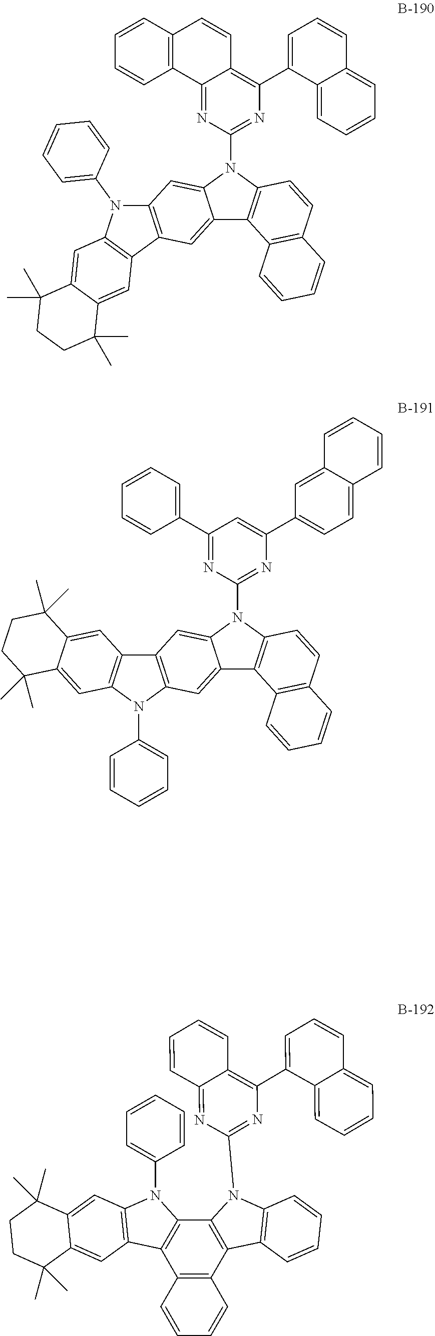 [SMILES: CC1(C)CCC(C)(C)c2cc3c(cc21)c1cc2c4c5ccccc5ccc4n(-c4nc(-c5cccc6ccccc56)c5ccc6ccccc6c5n4)c2cc1n3-c1ccccc1 ; CC1(C)CCC(C)(C)c2cc3c(cc21)c1cc2c(cc1n3-c1ccccc1)c1c3ccccc3ccc1n2-c1nc(-c2ccccc2)cc(-c2ccc3ccccc3c2)n1 ; CC1(C)CCC(C)(C)c2cc3c(cc21)c1c2ccccc2c2c4ccccc4n(-c4nc(-c5cccc6ccccc56)c5ccccc5n4)c2c1n3-c1ccccc1]

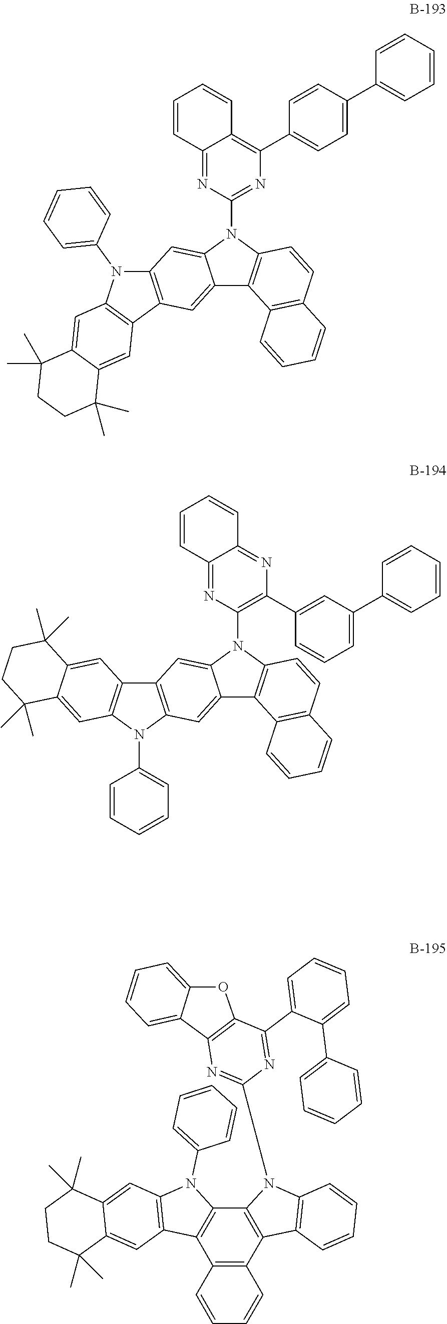 [SMILES: CC1(C)CCC(C)(C)c2cc3c(cc21)c1cc2c4c5ccccc5ccc4n(-c4nc(-c5ccc(-c6ccccc6)cc5)c5ccccc5n4)c2cc1n3-c1ccccc1 ; CC1(C)CCC(C)(C)c2cc3c(cc21)c1cc2c(cc1n3-c1ccccc1)c1c3ccccc3ccc1n2-c1nc2ccccc2nc1-c1cccc(-c2ccccc2)c1 ; CC1(C)CCC(C)(C)c2cc3c(cc21)c1c2ccccc2c2c4ccccc4n(-c4nc(-c5ccccc5-c5ccccc5)c5oc6ccccc6c5n4)c2c1n3-c1ccccc1]

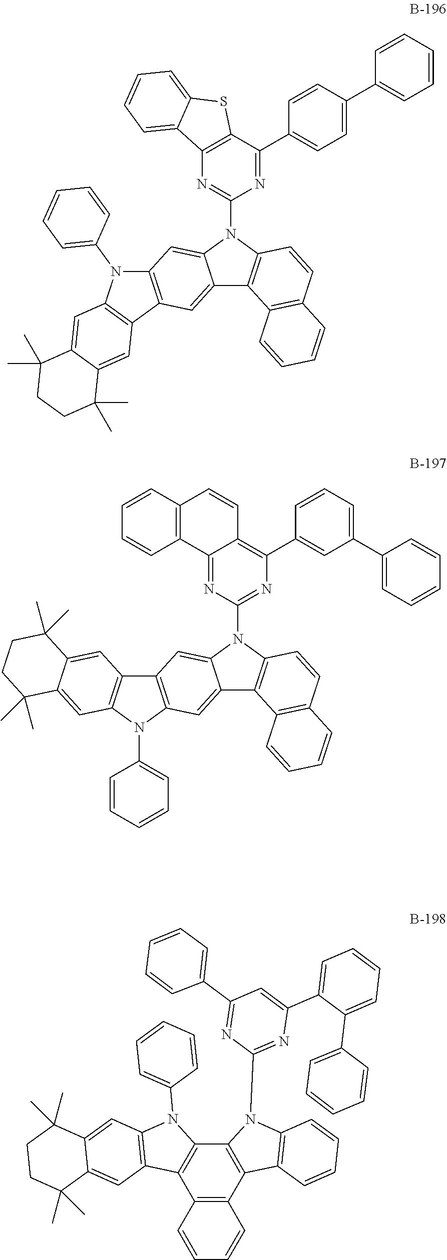 [SMILES: CC1(C)CCC(C)(C)c2cc3c(cc21)c1cc2c4c5ccccc5ccc4n(-c4nc(-c5ccc(-c6ccccc6)cc5)c5sc6ccccc6c5n4)c2cc1n3-c1ccccc1 ; CC1(C)CCC(C)(C)c2cc3c(cc21)c1cc2c(cc1n3-c1ccccc1)c1c3ccccc3ccc1n2-c1nc(-c2cccc(-c3ccccc3)c2)c2ccc3ccccc3c2n1 ; CC1(C)CCC(C)(C)c2cc3c(cc21)c1c2ccccc2c2c4ccccc4n(-c4nc(-c5ccccc5)cc(-c5ccccc5-c5ccccc5)n4)c2c1n3-c1ccccc1]

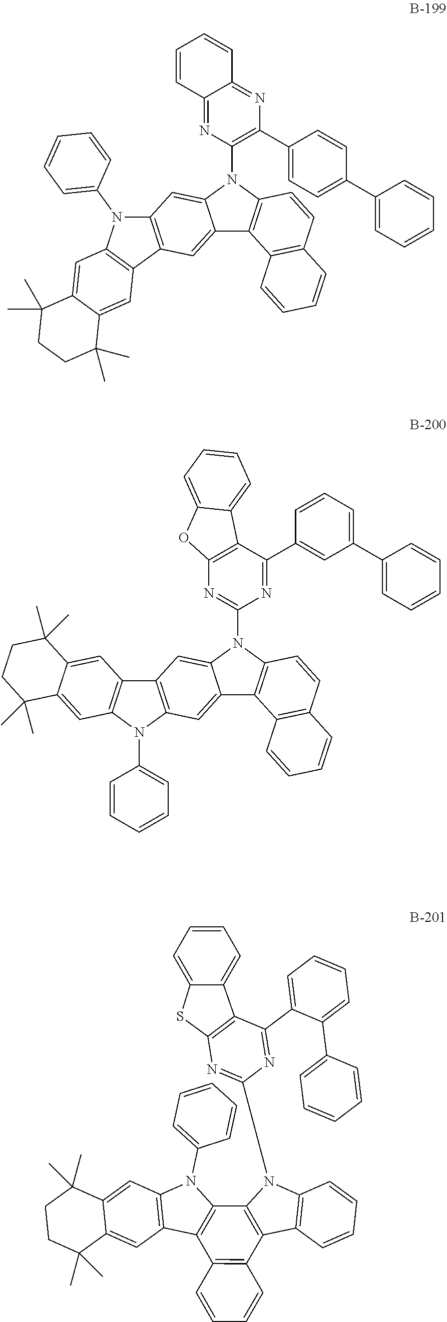 [SMILES: CC1(C)CCC(C)(C)c2cc3c(cc21)c1cc2c4c5ccccc5ccc4n(-c4nc5ccccc5nc4-c4ccc(-c5ccccc5)cc4)c2cc1n3-c1ccccc1 ; CC1(C)CCC(C)(C)c2cc3c(cc21)c1cc2c(cc1n3-c1ccccc1)c1c3ccccc3ccc1n2-c1nc(-c2cccc(-c3ccccc3)c2)c2c(n1)oc1ccccc12 ; CC1(C)CCC(C)(C)c2cc3c(cc21)c1c2ccccc2c2c4ccccc4n(-c4nc(-c5ccccc5-c5ccccc5)c5c(n4)sc4ccccc45)c2c1n3-c1ccccc1]

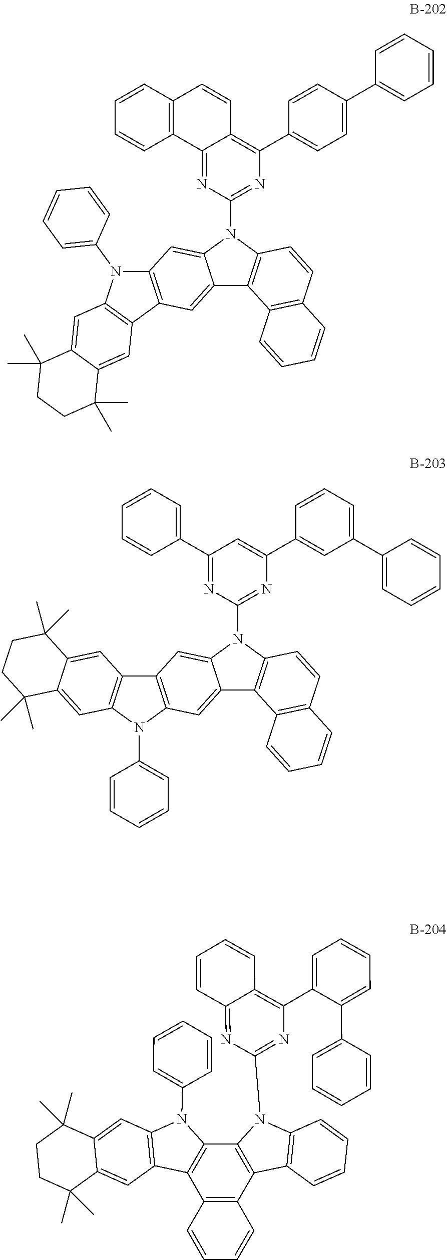 [SMILES: CC1(C)CCC(C)(C)c2cc3c(cc21)c1cc2c4c5ccccc5ccc4n(-c4nc(-c5ccc(-c6ccccc6)cc5)c5ccc6ccccc6c5n4)c2cc1n3-c1ccccc1 ; CC1(C)CCC(C)(C)c2cc3c(cc21)c1cc2c(cc1n3-c1ccccc1)c1c3ccccc3ccc1n2-c1nc(-c2ccccc2)cc(-c2cccc(-c3ccccc3)c2)n1 ; CC1(C)CCC(C)(C)c2cc3c(cc21)c1c2ccccc2c2c4ccccc4n(-c4nc(-c5ccccc5-c5ccccc5)c5ccccc5n4)c2c1n3-c1ccccc1]

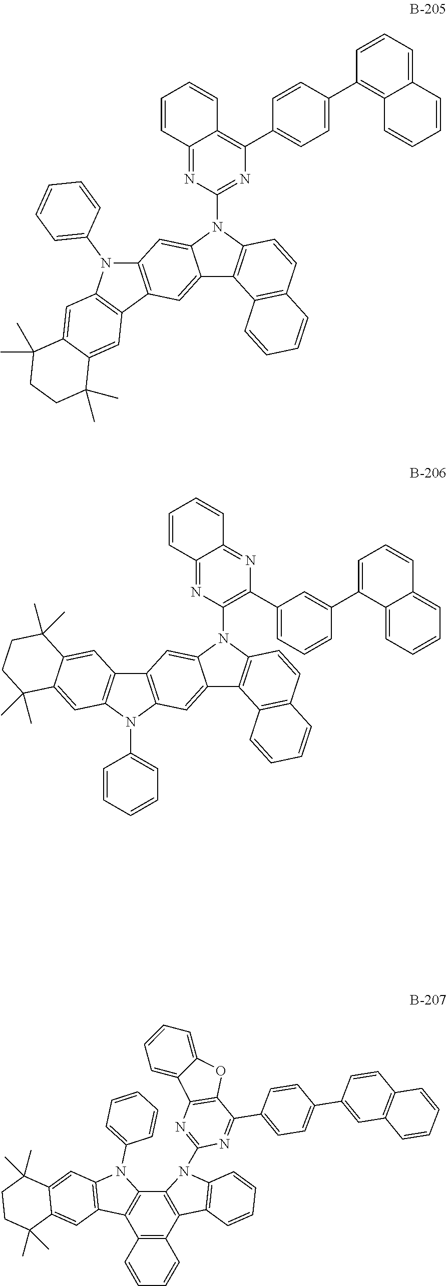 [SMILES: CC1(C)CCC(C)(C)c2cc3c(cc21)c1cc2c4c5ccccc5ccc4n(-c4nc(-c5ccc(-c6cccc7ccccc67)cc5)c5ccccc5n4)c2cc1n3-c1ccccc1 ; CC1(C)CCC(C)(C)c2cc3c(cc21)c1cc2c(cc1n3-c1ccccc1)c1c3ccccc3ccc1n2-c1nc2ccccc2nc1-c1cccc(-c2cccc3ccccc23)c1 ; CC1(C)CCC(C)(C)c2cc3c(cc21)c1c2ccccc2c2c4ccccc4n(-c4nc(-c5ccc(-c6ccc7ccccc7c6)cc5)c5oc6ccccc6c5n4)c2c1n3-c1ccccc1]

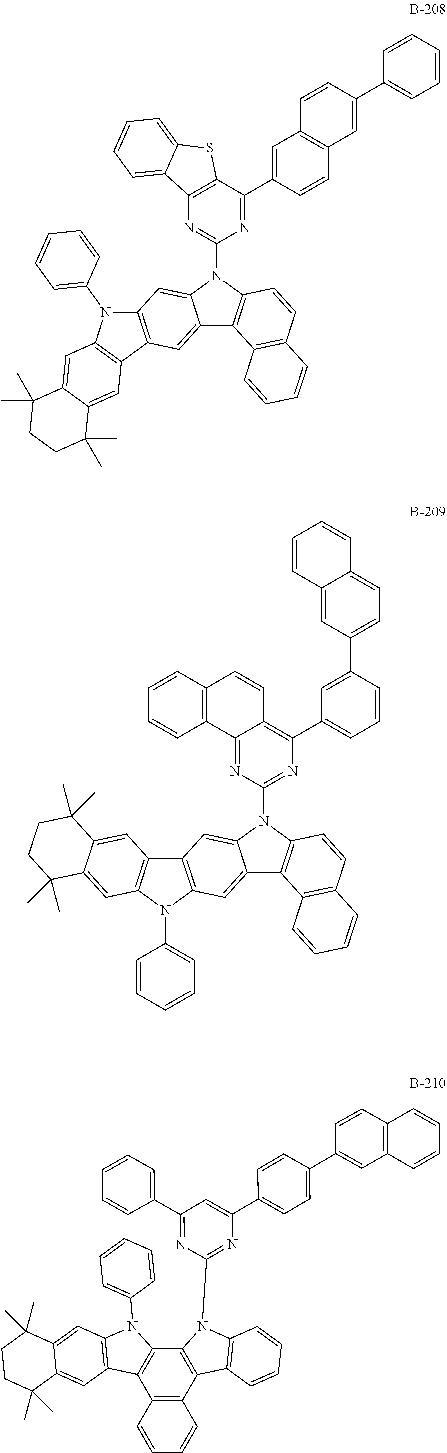 [SMILES: CC1(C)CCC(C)(C)c2cc3c(cc21)c1cc2c4c5ccccc5ccc4n(-c4nc(-c5ccc6cc(-c7ccccc7)ccc6c5)c5sc6ccccc6c5n4)c2cc1n3-c1ccccc1 ; CC1(C)CCC(C)(C)c2cc3c(cc21)c1cc2c(cc1n3-c1ccccc1)c1c3ccccc3ccc1n2-c1nc(-c2cccc(-c3ccc4ccccc4c3)c2)c2ccc3ccccc3c2n1 ; CC1(C)CCC(C)(C)c2cc3c(cc21)c1c2ccccc2c2c4ccccc4n(-c4nc(-c5ccccc5)cc(-c5ccc(-c6ccc7ccccc7c6)cc5)n4)c2c1n3-c1ccccc1]

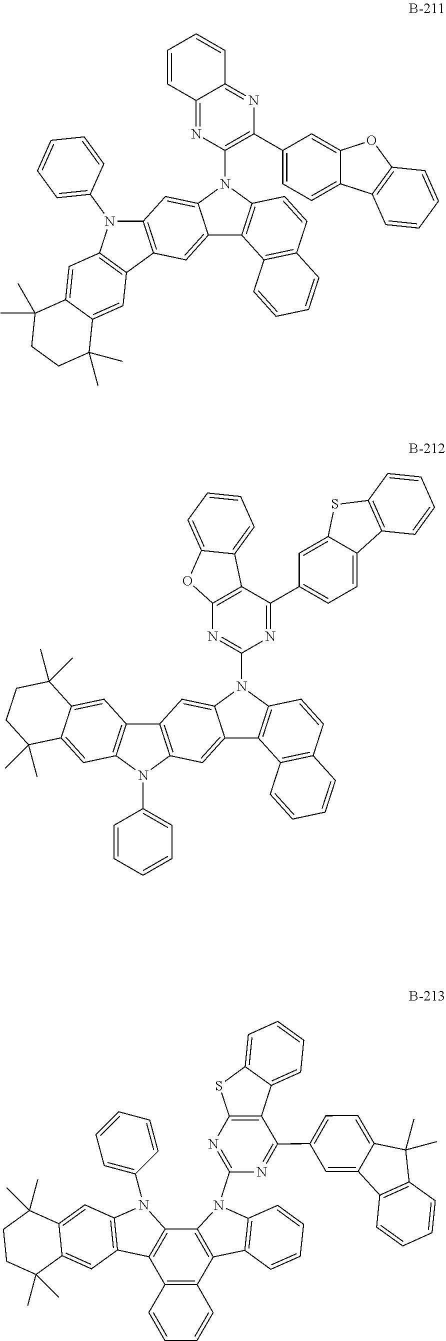 [SMILES: CC1(C)CCC(C)(C)c2cc3c(cc21)c1cc2c4c5ccccc5ccc4n(-c4nc5ccccc5nc4-c4ccc5c(c4)oc4ccccc45)c2cc1n3-c1ccccc1 ; CC1(C)CCC(C)(C)c2cc3c(cc21)c1cc2c(cc1n3-c1ccccc1)c1c3ccccc3ccc1n2-c1nc(-c2ccc3c(c2)sc2ccccc23)c2c(n1)oc1ccccc12 ; CC1(C)CCC(C)(C)c2cc3c(cc21)c1c2ccccc2c2c4ccccc4n(-c4nc(-c5ccc6c(c5)-c5ccccc5C6(C)C)c5c(n4)sc4ccccc45)c2c1n3-c1ccccc1]

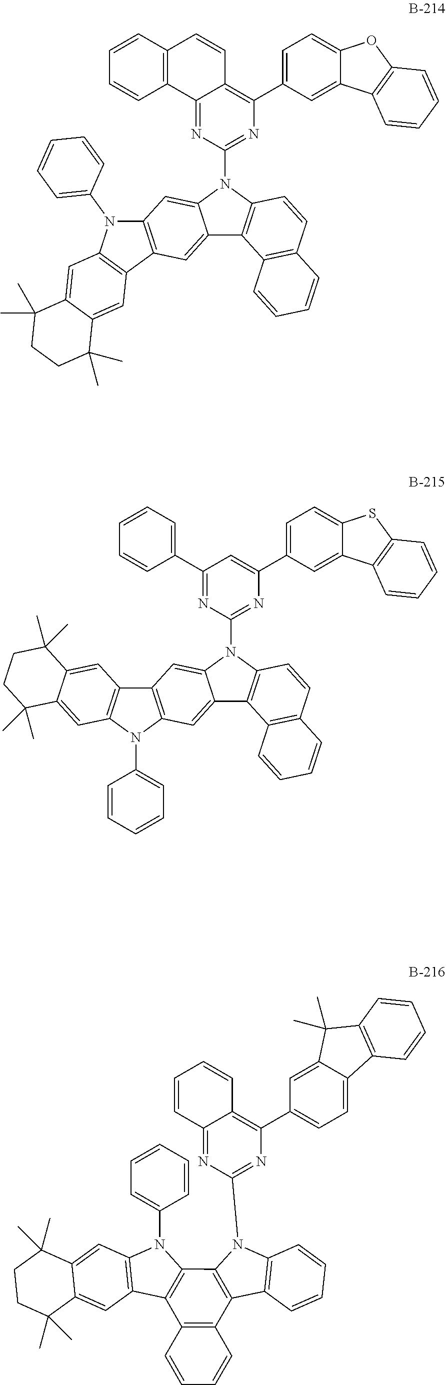 [SMILES: CC1(C)CCC(C)(C)c2cc3c(cc21)c1cc2c4c5ccccc5ccc4n(-c4nc(-c5ccc6oc7ccccc7c6c5)c5ccc6ccccc6c5n4)c2cc1n3-c1ccccc1 ; CC1(C)CCC(C)(C)c2cc3c(cc21)c1cc2c(cc1n3-c1ccccc1)c1c3ccccc3ccc1n2-c1nc(-c2ccccc2)cc(-c2ccc3sc4ccccc4c3c2)n1 ; CC1(C)CCC(C)(C)c2cc3c(cc21)c1c2ccccc2c2c4ccccc4n(-c4nc(-c5ccc6c(c5)C(C)(C)c5ccccc5-6)c5ccccc5n4)c2c1n3-c1ccccc1]

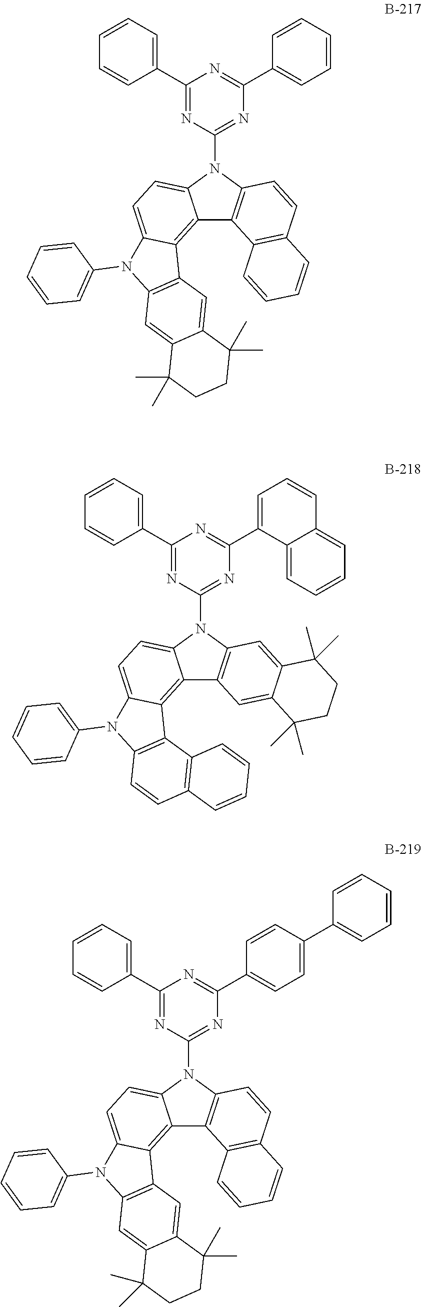 [SMILES: CC1(C)CCC(C)(C)c2cc3c(cc21)c1c2c4c5ccccc5ccc4n(-c4nc(-c5ccccc5)nc(-c5ccccc5)n4)c2ccc1n3-c1ccccc1 ; CC1(C)CCC(C)(C)c2cc3c(cc21)c1c2c4c5ccccc5ccc4n(-c4ccccc4)c2ccc1n3-c1nc(-c2ccccc2)nc(-c2cccc3ccccc23)n1 ; CC1(C)CCC(C)(C)c2cc3c(cc21)c1c2c4c5ccccc5ccc4n(-c4nc(-c5ccccc5)nc(-c5ccc(-c6ccccc6)cc5)n4)c2ccc1n3-c1ccccc1]

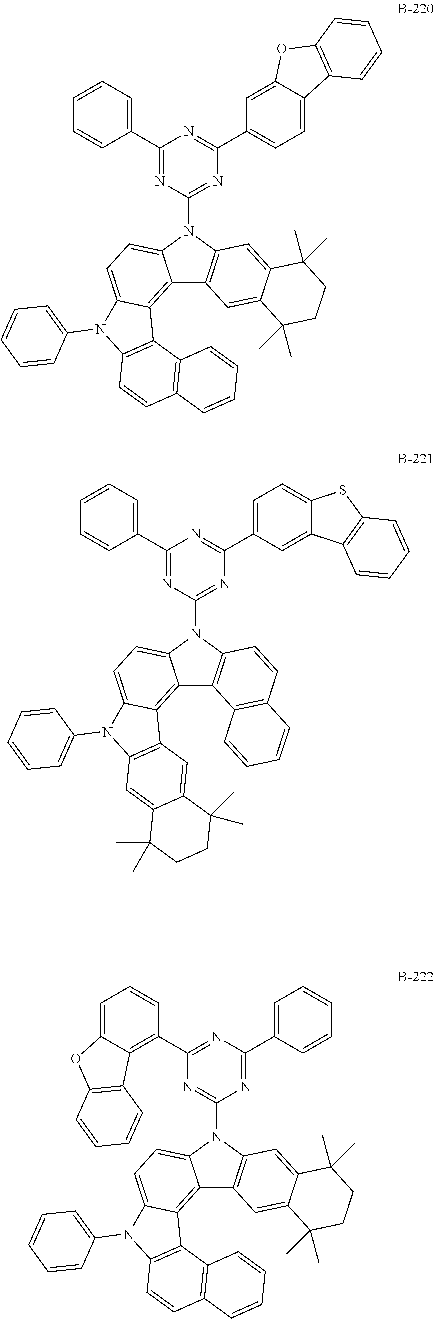 [SMILES: CC1(C)CCC(C)(C)c2cc3c(cc21)c1c2c4c5ccccc5ccc4n(-c4ccccc4)c2ccc1n3-c1nc(-c2ccccc2)nc(-c2ccc3c(c2)oc2ccccc23)n1 ; CC1(C)CCC(C)(C)c2cc3c(cc21)c1c2c4c5ccccc5ccc4n(-c4nc(-c5ccccc5)nc(-c5ccc6sc7ccccc7c6c5)n4)c2ccc1n3-c1ccccc1 ; CC1(C)CCC(C)(C)c2cc3c(cc21)c1c2c4c5ccccc5ccc4n(-c4ccccc4)c2ccc1n3-c1nc(-c2ccccc2)nc(-c2cccc3oc4ccccc4c23)n1]

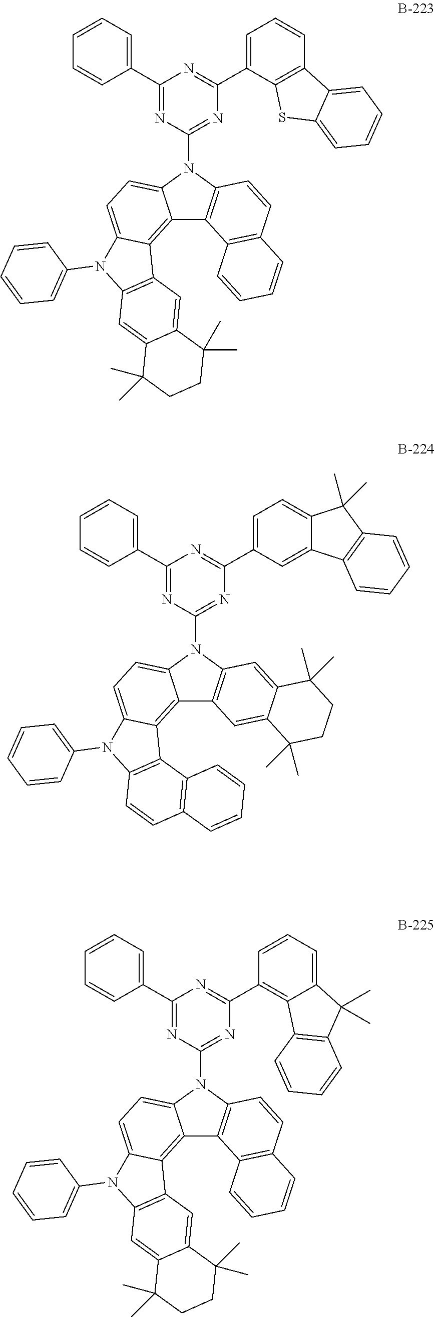 [SMILES: CC1(C)CCC(C)(C)c2cc3c(cc21)c1c2c4c5ccccc5ccc4n(-c4nc(-c5ccccc5)nc(-c5cccc6c5sc5ccccc56)n4)c2ccc1n3-c1ccccc1 ; CC1(C)CCC(C)(C)c2cc3c(cc21)c1c2c4c5ccccc5ccc4n(-c4ccccc4)c2ccc1n3-c1nc(-c2ccccc2)nc(-c2ccc3c(c2)-c2ccccc2C3(C)C)n1 ; CC1(C)CCC(C)(C)c2cc3c(cc21)c1c2c4c5ccccc5ccc4n(-c4nc(-c5ccccc5)nc(-c5cccc6c5-c5ccccc5C6(C)C)n4)c2ccc1n3-c1ccccc1]

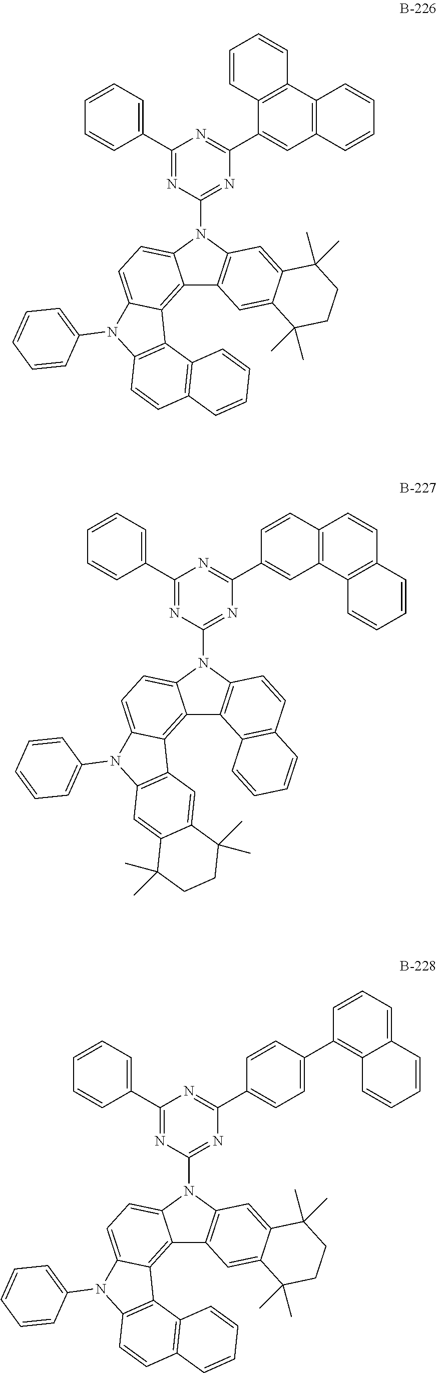 [SMILES: CC1(C)CCC(C)(C)c2cc3c(cc21)c1c2c4c5ccccc5ccc4n(-c4ccccc4)c2ccc1n3-c1nc(-c2ccccc2)nc(-c2cc3ccccc3c3ccccc23)n1 ; CC1(C)CCC(C)(C)c2cc3c(cc21)c1c2c4c5ccccc5ccc4n(-c4nc(-c5ccccc5)nc(-c5ccc6ccc7ccccc7c6c5)n4)c2ccc1n3-c1ccccc1 ; CC1(C)CCC(C)(C)c2cc3c(cc21)c1c2c4c5ccccc5ccc4n(-c4ccccc4)c2ccc1n3-c1nc(-c2ccccc2)nc(-c2ccc(-c3cccc4ccccc34)cc2)n1]

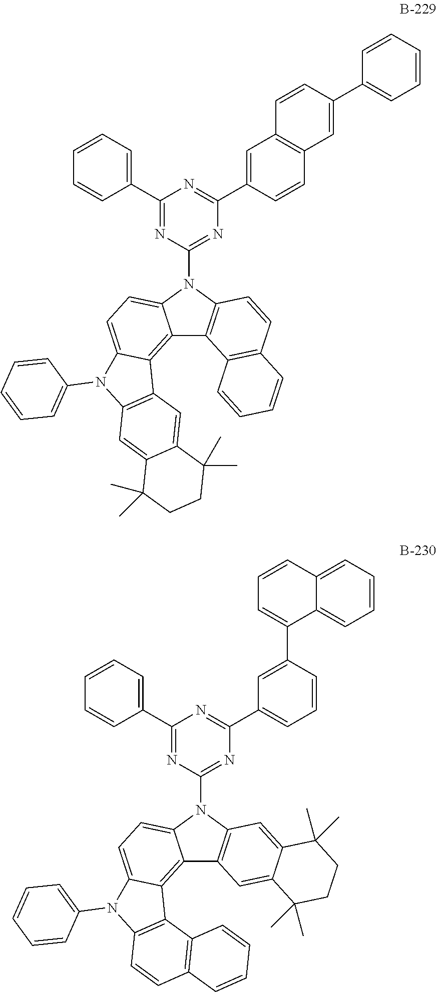 [SMILES: CC1(C)CCC(C)(C)c2cc3c(cc21)c1c2c4c5ccccc5ccc4n(-c4nc(-c5ccccc5)nc(-c5ccc6cc(-c7ccccc7)ccc6c5)n4)c2ccc1n3-c1ccccc1 ; CC1(C)CCC(C)(C)c2cc3c(cc21)c1c2c4c5ccccc5ccc4n(-c4ccccc4)c2ccc1n3-c1nc(-c2ccccc2)nc(-c2cccc(-c3cccc4ccccc34)c2)n1]

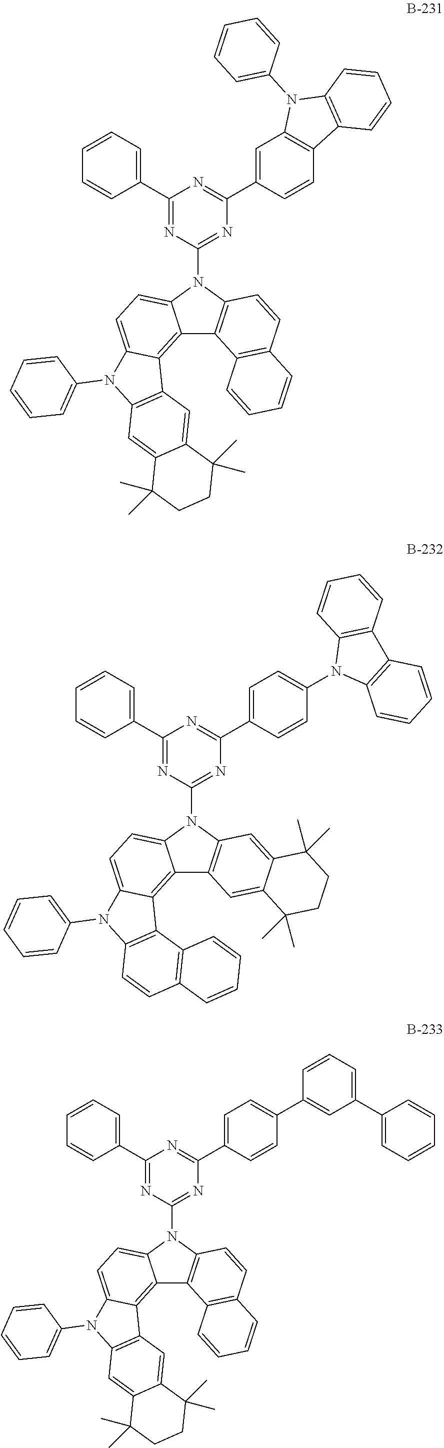 [SMILES: CC1(C)CCC(C)(C)c2cc3c(cc21)c1c2c4c5ccccc5ccc4n(-c4nc(-c5ccccc5)nc(-c5ccc6c7ccccc7n(-c7ccccc7)c6c5)n4)c2ccc1n3-c1ccccc1 ; CC1(C)CCC(C)(C)c2cc3c(cc21)c1c2c4c5ccccc5ccc4n(-c4ccccc4)c2ccc1n3-c1nc(-c2ccccc2)nc(-c2ccc(-n3c4ccccc4c4ccccc43)cc2)n1 ; CC1(C)CCC(C)(C)c2cc3c(cc21)c1c2c4c5ccccc5ccc4n(-c4nc(-c5ccccc5)nc(-c5ccc(-c6cccc(-c7ccccc7)c6)cc5)n4)c2ccc1n3-c1ccccc1]

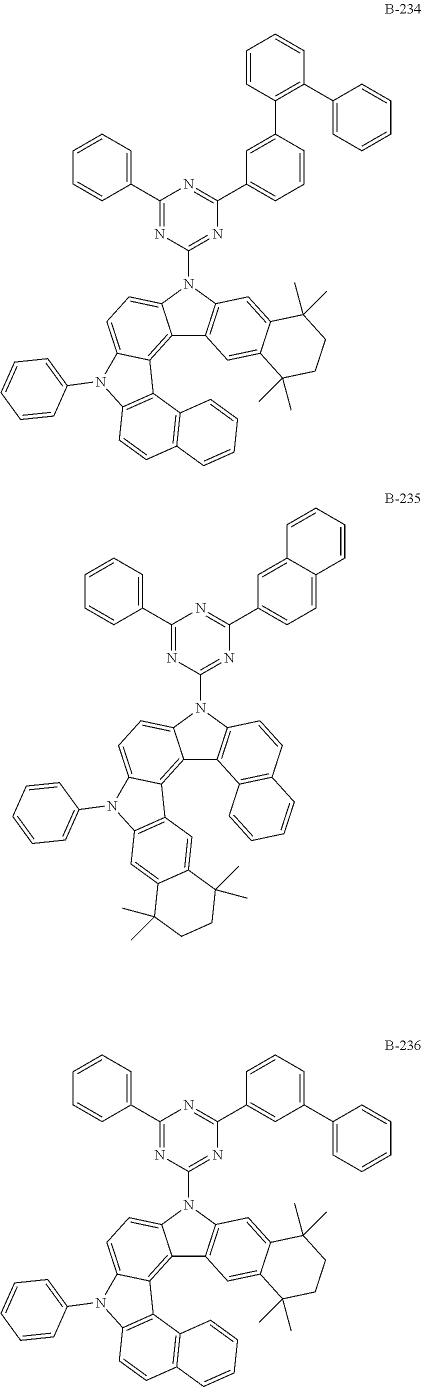 [SMILES: CC1(C)CCC(C)(C)c2cc3c(cc21)c1c2c4c5ccccc5ccc4n(-c4ccccc4)c2ccc1n3-c1nc(-c2ccccc2)nc(-c2cccc(-c3ccccc3-c3ccccc3)c2)n1 ; CC1(C)CCC(C)(C)c2cc3c(cc21)c1c2c4c5ccccc5ccc4n(-c4nc(-c5ccccc5)nc(-c5ccc6ccccc6c5)n4)c2ccc1n3-c1ccccc1 ; CC1(C)CCC(C)(C)c2cc3c(cc21)c1c2c4c5ccccc5ccc4n(-c4ccccc4)c2ccc1n3-c1nc(-c2ccccc2)nc(-c2cccc(-c3ccccc3)c2)n1]

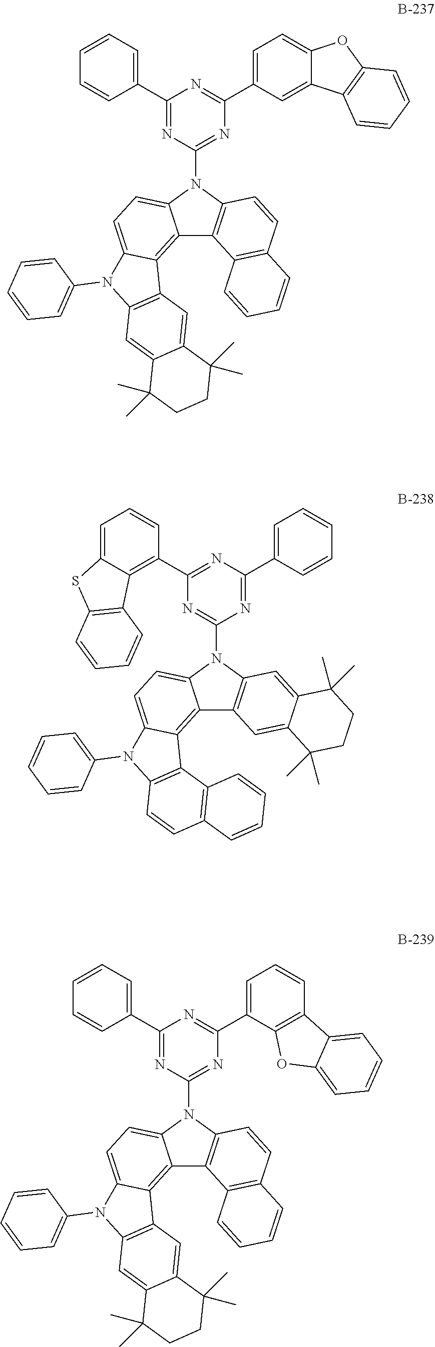 [SMILES: CC1(C)CCC(C)(C)c2cc3c(cc21)c1c2c4c5ccccc5ccc4n(-c4nc(-c5ccccc5)nc(-c5ccc6oc7ccccc7c6c5)n4)c2ccc1n3-c1ccccc1 ; CC1(C)CCC(C)(C)c2cc3c(cc21)c1c2c4c5ccccc5ccc4n(-c4ccccc4)c2ccc1n3-c1nc(-c2ccccc2)nc(-c2cccc3sc4ccccc4c23)n1 ; CC1(C)CCC(C)(C)c2cc3c(cc21)c1c2c4c5ccccc5ccc4n(-c4nc(-c5ccccc5)nc(-c5cccc6c5oc5ccccc56)n4)c2ccc1n3-c1ccccc1]

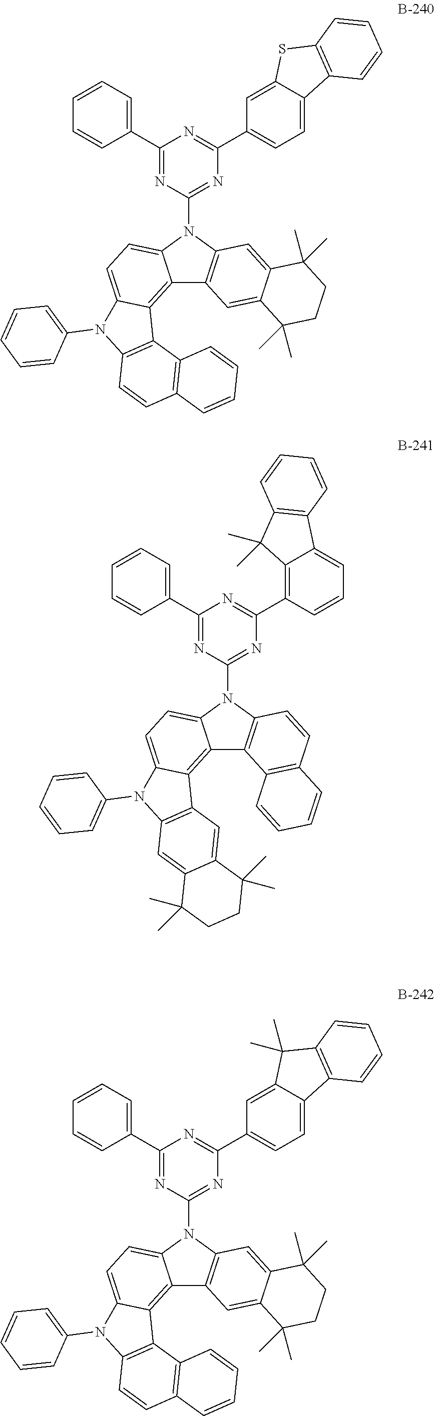 [SMILES: CC1(C)CCC(C)(C)c2cc3c(cc21)c1c2c4c5ccccc5ccc4n(-c4ccccc4)c2ccc1n3-c1nc(-c2ccccc2)nc(-c2ccc3c(c2)sc2ccccc23)n1 ; CC1(C)CCC(C)(C)c2cc3c(cc21)c1c2c4c5ccccc5ccc4n(-c4nc(-c5ccccc5)nc(-c5cccc6c5C(C)(C)c5ccccc5-6)n4)c2ccc1n3-c1ccccc1 ; CC1(C)CCC(C)(C)c2cc3c(cc21)c1c2c4c5ccccc5ccc4n(-c4ccccc4)c2ccc1n3-c1nc(-c2ccccc2)nc(-c2ccc3c(c2)C(C)(C)c2ccccc2-3)n1]

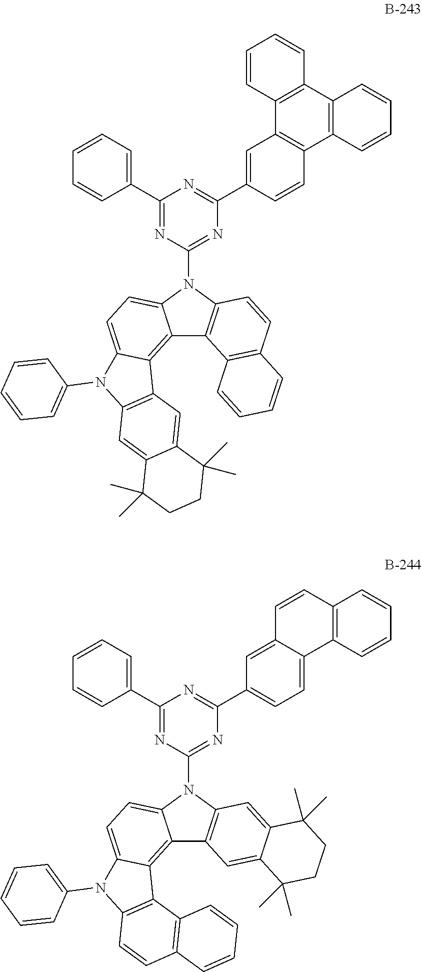 [SMILES: CC1(C)CCC(C)(C)c2cc3c(cc21)c1c2c4c5ccccc5ccc4n(-c4nc(-c5ccccc5)nc(-c5ccc6c7ccccc7c7ccccc7c6c5)n4)c2ccc1n3-c1ccccc1 ; CC1(C)CCC(C)(C)c2cc3c(cc21)c1c2c4c5ccccc5ccc4n(-c4ccccc4)c2ccc1n3-c1nc(-c2ccccc2)nc(-c2ccc3c(ccc4ccccc43)c2)n1]

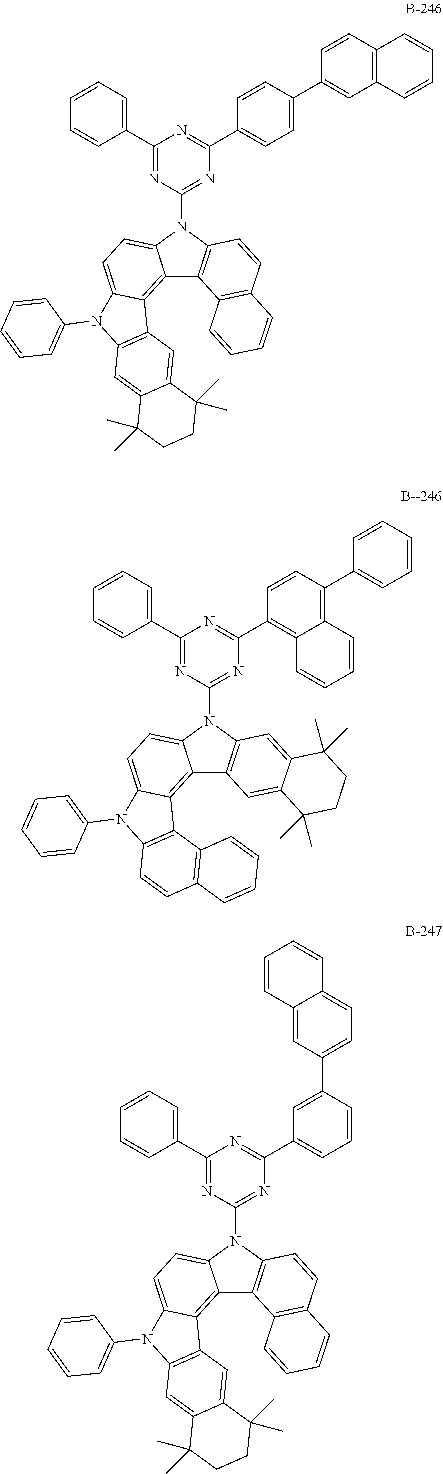 [SMILES: CC1(C)CCC(C)(C)c2cc3c(cc21)c1c2c4c5ccccc5ccc4n(-c4nc(-c5ccccc5)nc(-c5ccc(-c6ccc7ccccc7c6)cc5)n4)c2ccc1n3-c1ccccc1 ; CC1(C)CCC(C)(C)c2cc3c(cc21)c1c2c4c5ccccc5ccc4n(-c4ccccc4)c2ccc1n3-c1nc(-c2ccccc2)nc(-c2ccc(-c3ccccc3)c3ccccc23)n1 ; CC1(C)CCC(C)(C)c2cc3c(cc21)c1c2c4c5ccccc5ccc4n(-c4nc(-c5ccccc5)nc(-c5cccc(-c6ccc7ccccc7c6)c5)n4)c2ccc1n3-c1ccccc1]

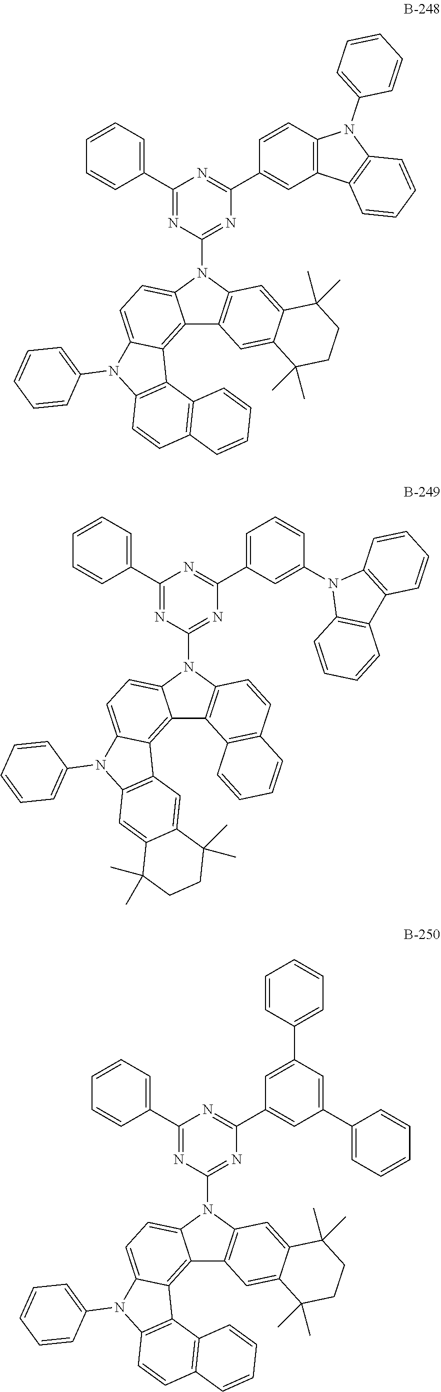 [SMILES: CC1(C)CCC(C)(C)c2cc3c(cc21)c1c2c4c5ccccc5ccc4n(-c4ccccc4)c2ccc1n3-c1nc(-c2ccccc2)nc(-c2ccc3c(c2)c2ccccc2n3-c2ccccc2)n1 ; CC1(C)CCC(C)(C)c2cc3c(cc21)c1c2c4c5ccccc5ccc4n(-c4nc(-c5ccccc5)nc(-c5cccc(-n6c7ccccc7c7ccccc76)c5)n4)c2ccc1n3-c1ccccc1 ; CC1(C)CCC(C)(C)c2cc3c(cc21)c1c2c4c5ccccc5ccc4n(-c4ccccc4)c2ccc1n3-c1nc(-c2ccccc2)nc(-c2cc(-c3ccccc3)cc(-c3ccccc3)c2)n1]

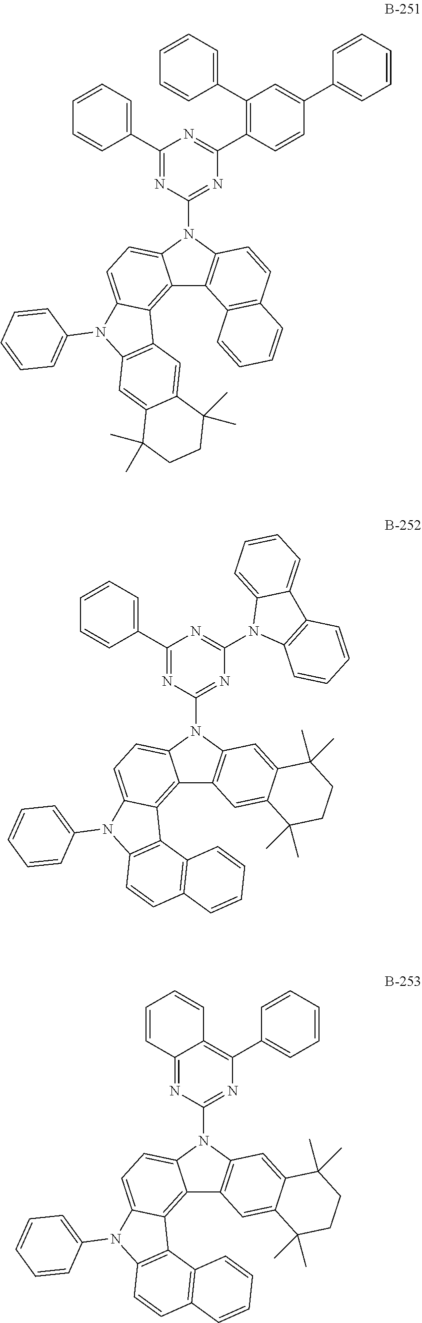 [SMILES: CC1(C)CCC(C)(C)c2cc3c(cc21)c1c2c4c5ccccc5ccc4n(-c4nc(-c5ccccc5)nc(-c5ccc(-c6ccccc6)cc5-c5ccccc5)n4)c2ccc1n3-c1ccccc1 ; CC1(C)CCC(C)(C)c2cc3c(cc21)c1c2c4c5ccccc5ccc4n(-c4ccccc4)c2ccc1n3-c1nc(-c2ccccc2)nc(-n2c3ccccc3c3ccccc32)n1 ; CC1(C)CCC(C)(C)c2cc3c(cc21)c1c2c4c5ccccc5ccc4n(-c4ccccc4)c2ccc1n3-c1nc(-c2ccccc2)c2ccccc2n1]

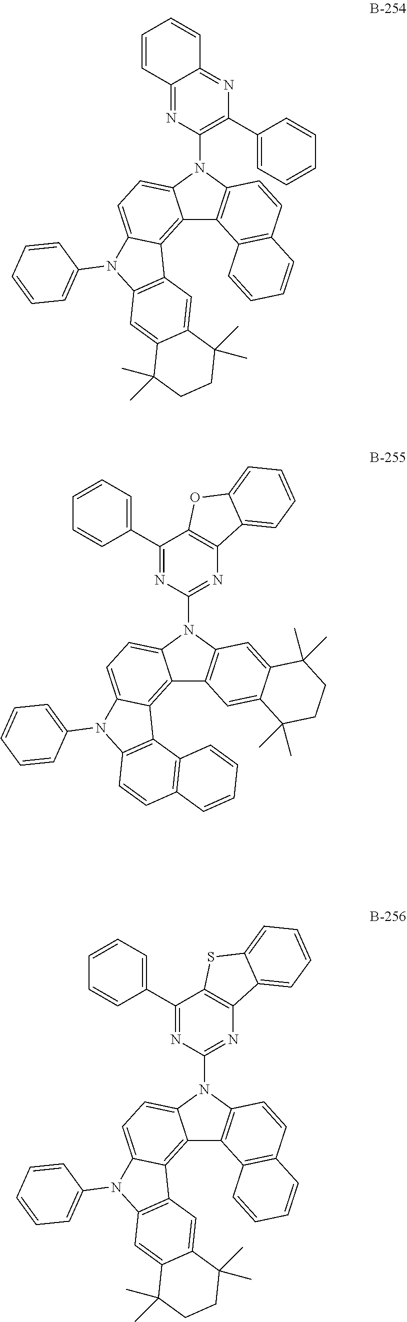 [SMILES: CC1(C)CCC(C)(C)c2cc3c(cc21)c1c2c4c5ccccc5ccc4n(-c4nc5ccccc5nc4-c4ccccc4)c2ccc1n3-c1ccccc1 ; CC1(C)CCC(C)(C)c2cc3c(cc21)c1c2c4c5ccccc5ccc4n(-c4ccccc4)c2ccc1n3-c1nc(-c2ccccc2)c2oc3ccccc3c2n1 ; CC1(C)CCC(C)(C)c2cc3c(cc21)c1c2c4c5ccccc5ccc4n(-c4nc(-c5ccccc5)c5sc6ccccc6c5n4)c2ccc1n3-c1ccccc1]

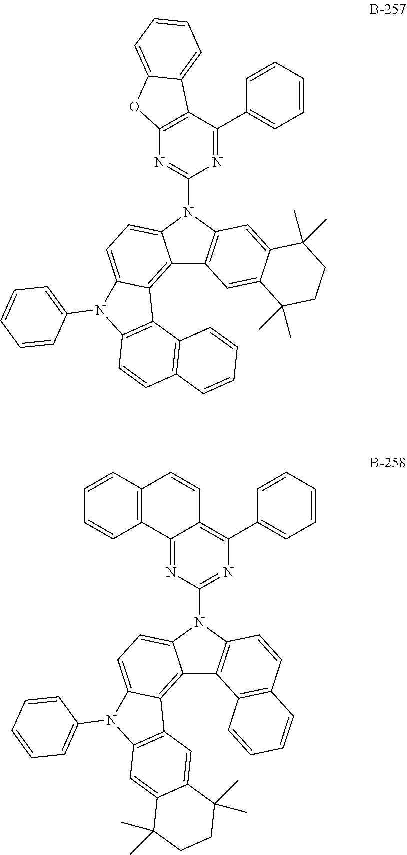 [SMILES: CC1(C)CCC(C)(C)c2cc3c(cc21)c1c2c4c5ccccc5ccc4n(-c4ccccc4)c2ccc1n3-c1nc(-c2ccccc2)c2c(n1)oc1ccccc12 ; CC1(C)CCC(C)(C)c2cc3c(cc21)c1c2c4c5ccccc5ccc4n(-c4nc(-c5ccccc5)c5ccc6ccccc6c5n4)c2ccc1n3-c1ccccc1]

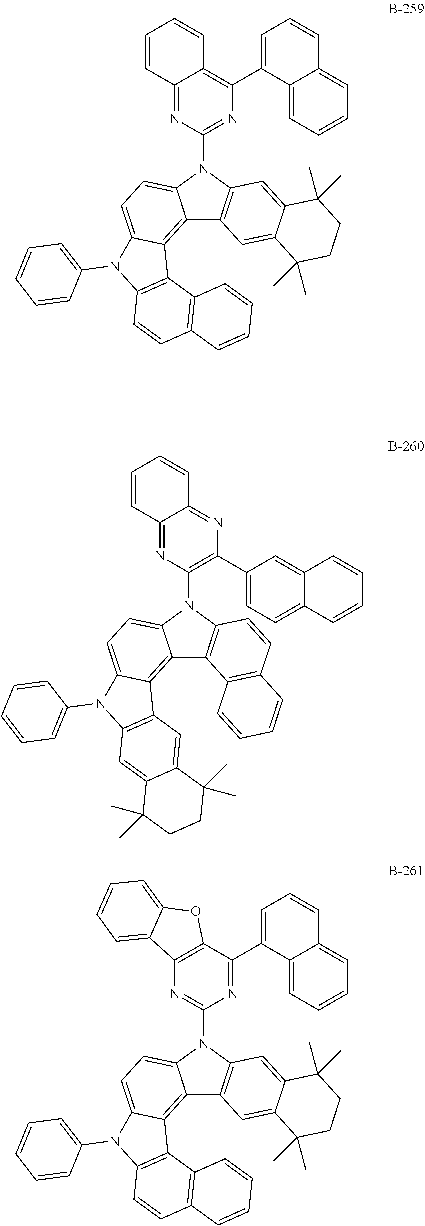

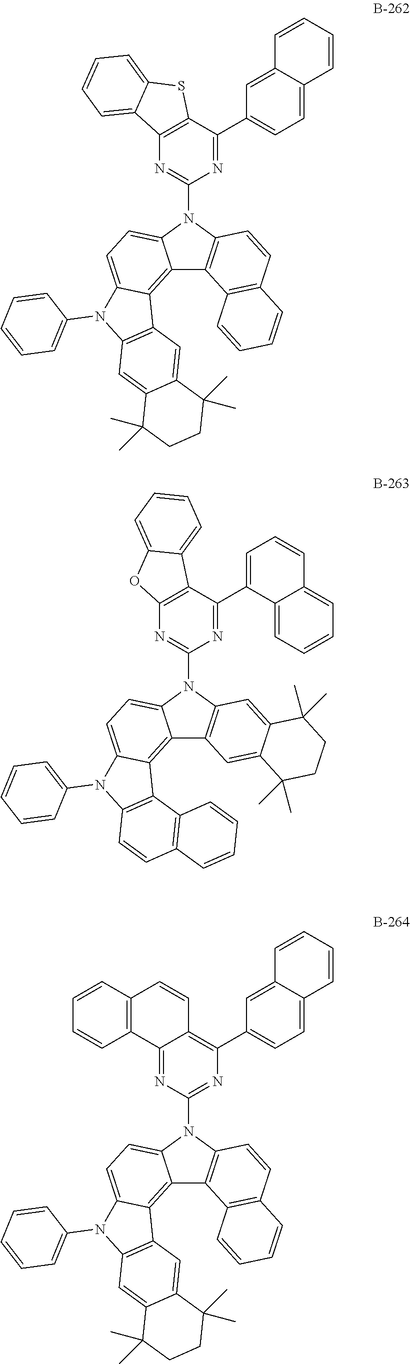 [SMILES: CC1(C)CCC(C)(C)c2cc3c(cc21)c1c2c4c5ccccc5ccc4n(-c4nc(-c5ccc6ccccc6c5)c5sc6ccccc6c5n4)c2ccc1n3-c1ccccc1 ; CC1(C)CCC(C)(C)c2cc3c(cc21)c1c2c4c5ccccc5ccc4n(-c4ccccc4)c2ccc1n3-c1nc(-c2cccc3ccccc23)c2c(n1)oc1ccccc12 ; CC1(C)CCC(C)(C)c2cc3c(cc21)c1c2c4c5ccccc5ccc4n(-c4nc(-c5ccc6ccccc6c5)c5ccc6ccccc6c5n4)c2ccc1n3-c1ccccc1]

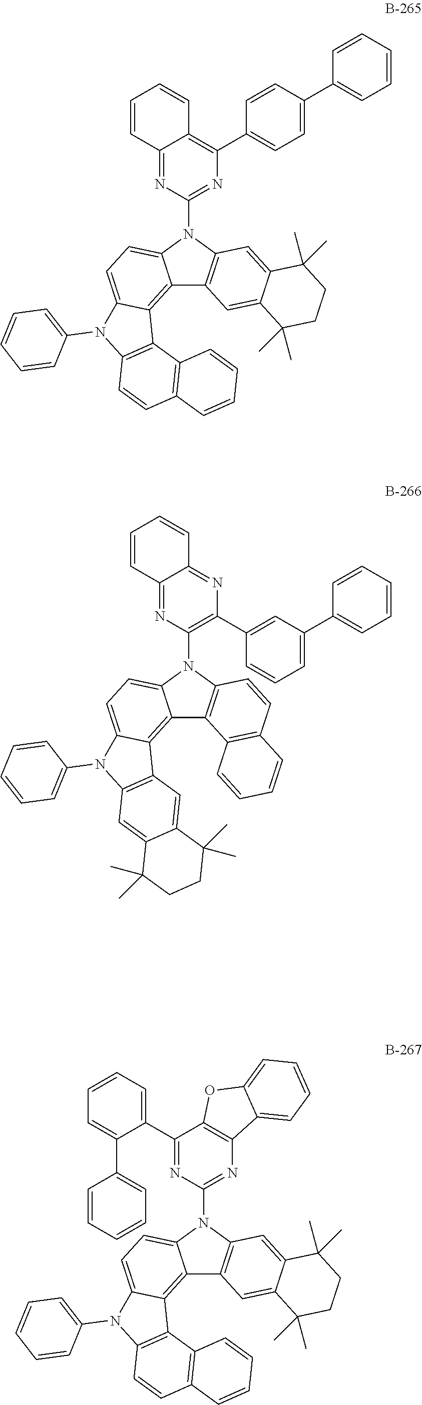 [SMILES: CC1(C)CCC(C)(C)c2cc3c(cc21)c1c2c4c5ccccc5ccc4n(-c4ccccc4)c2ccc1n3-c1nc(-c2ccc(-c3ccccc3)cc2)c2ccccc2n1 ; CC1(C)CCC(C)(C)c2cc3c(cc21)c1c2c4c5ccccc5ccc4n(-c4nc5ccccc5nc4-c4cccc(-c5ccccc5)c4)c2ccc1n3-c1ccccc1 ; CC1(C)CCC(C)(C)c2cc3c(cc21)c1c2c4c5ccccc5ccc4n(-c4ccccc4)c2ccc1n3-c1nc(-c2ccccc2-c2ccccc2)c2oc3ccccc3c2n1]

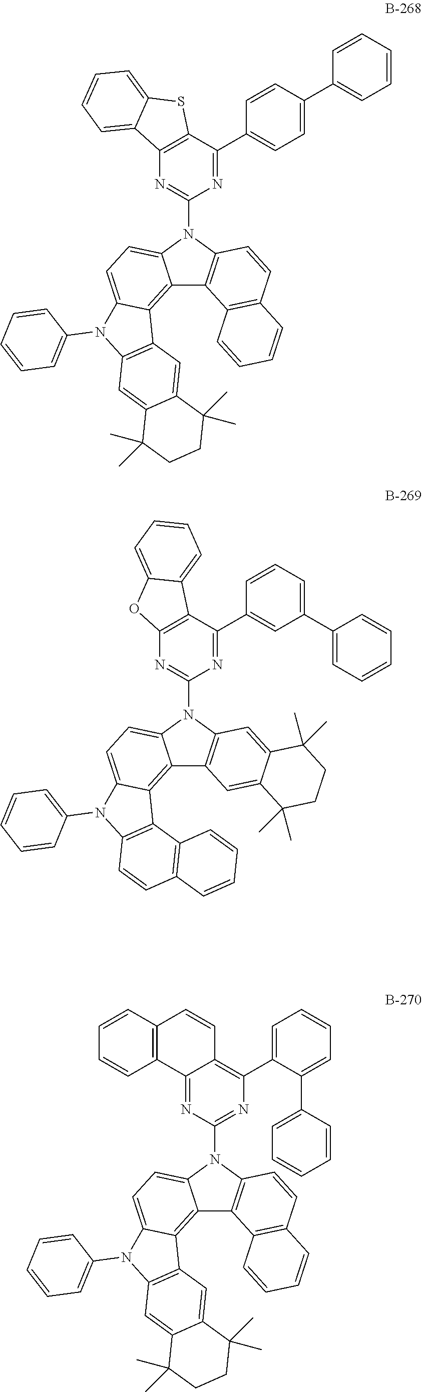 [SMILES: CC1(C)CCC(C)(C)c2cc3c(cc21)c1c2c4c5ccccc5ccc4n(-c4nc(-c5ccc(-c6ccccc6)cc5)c5sc6ccccc6c5n4)c2ccc1n3-c1ccccc1 ; CC1(C)CCC(C)(C)c2cc3c(cc21)c1c2c4c5ccccc5ccc4n(-c4ccccc4)c2ccc1n3-c1nc(-c2cccc(-c3ccccc3)c2)c2c(n1)oc1ccccc12 ; CC1(C)CCC(C)(C)c2cc3c(cc21)c1c2c4c5ccccc5ccc4n(-c4nc(-c5ccccc5-c5ccccc5)c5ccc6ccccc6c5n4)c2ccc1n3-c1ccccc1]

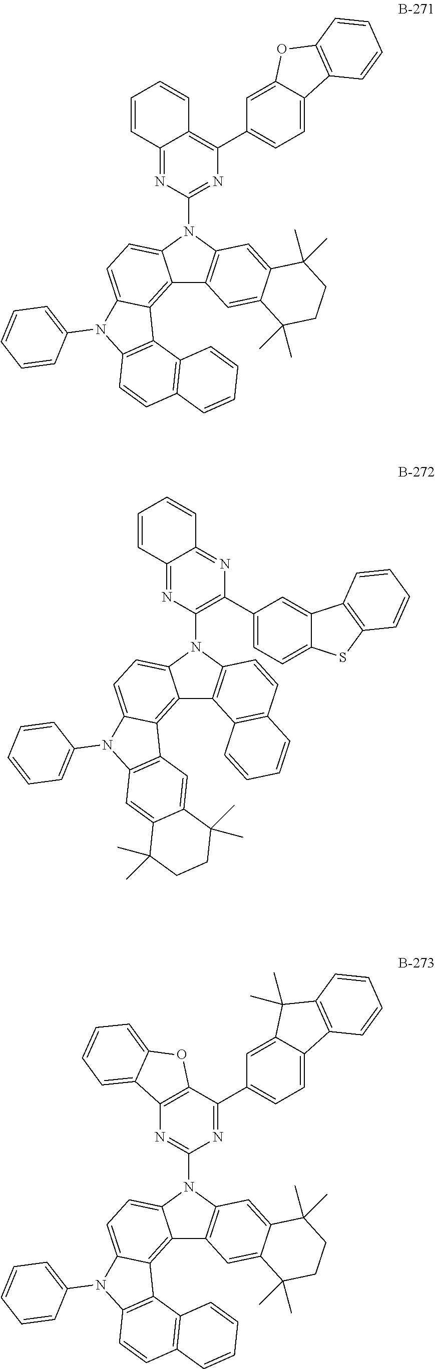 [SMILES: CC1(C)CCC(C)(C)c2cc3c(cc21)c1c2c4c5ccccc5ccc4n(-c4ccccc4)c2ccc1n3-c1nc(-c2ccc3c(c2)oc2ccccc23)c2ccccc2n1 ; CC1(C)CCC(C)(C)c2cc3c(cc21)c1c2c4c5ccccc5ccc4n(-c4nc5ccccc5nc4-c4ccc5sc6ccccc6c5c4)c2ccc1n3-c1ccccc1 ; CC1(C)CCC(C)(C)c2cc3c(cc21)c1c2c4c5ccccc5ccc4n(-c4ccccc4)c2ccc1n3-c1nc(-c2ccc3c(c2)C(C)(C)c2ccccc2-3)c2oc3ccccc3c2n1]

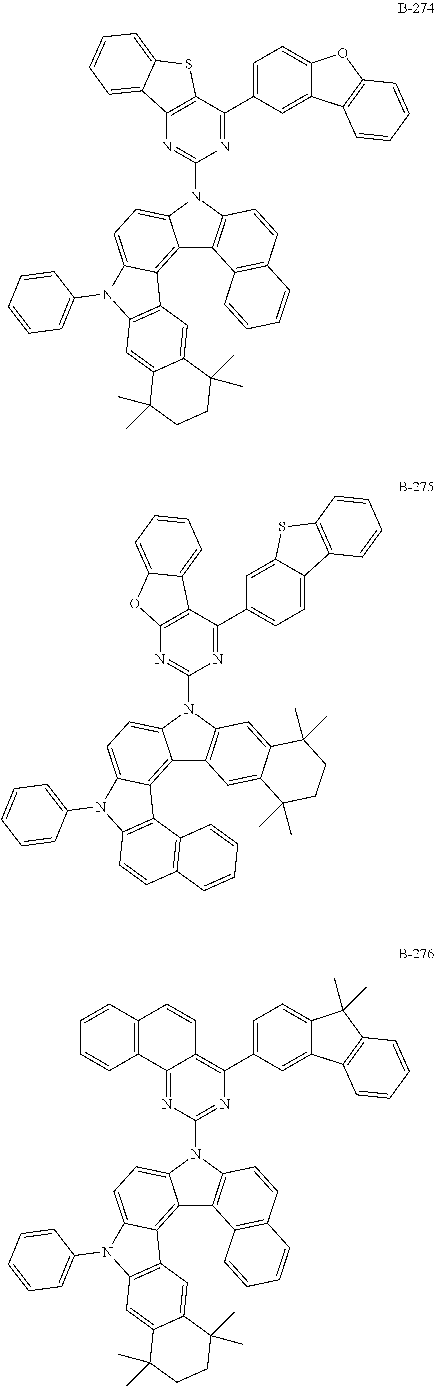 [SMILES: CC1(C)CCC(C)(C)c2cc3c(cc21)c1c2c4c5ccccc5ccc4n(-c4nc(-c5ccc6oc7ccccc7c6c5)c5sc6ccccc6c5n4)c2ccc1n3-c1ccccc1 ; CC1(C)CCC(C)(C)c2cc3c(cc21)c1c2c4c5ccccc5ccc4n(-c4ccccc4)c2ccc1n3-c1nc(-c2ccc3c(c2)sc2ccccc23)c2c(n1)oc1ccccc12 ; CC1(C)CCC(C)(C)c2cc3c(cc21)c1c2c4c5ccccc5ccc4n(-c4nc(-c5ccc6c(c5)-c5ccccc5C6(C)C)c5ccc6ccccc6c5n4)c2ccc1n3-c1ccccc1]

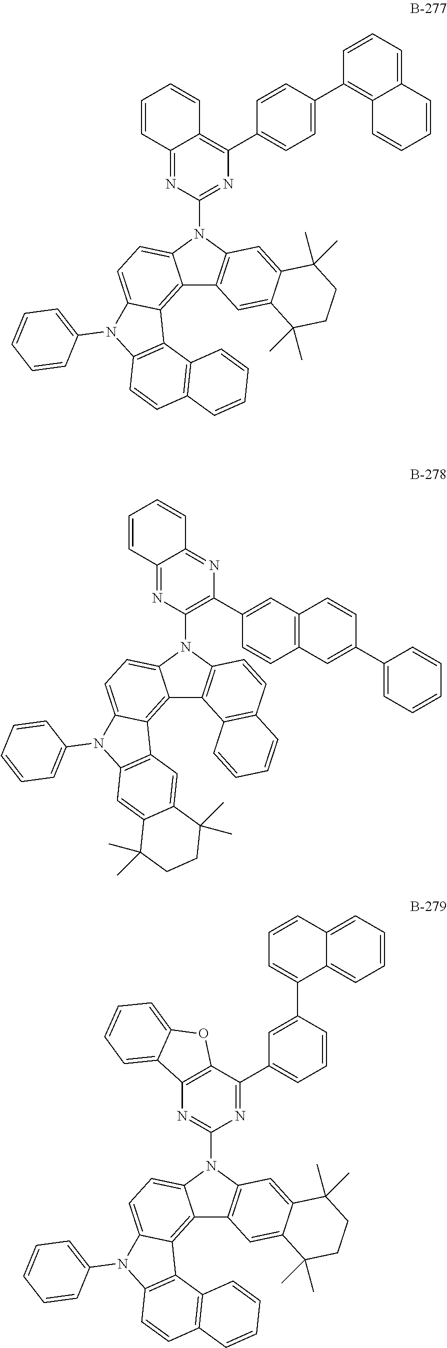 [SMILES: CC1(C)CCC(C)(C)c2cc3c(cc21)c1c2c4c5ccccc5ccc4n(-c4ccccc4)c2ccc1n3-c1nc(-c2ccc(-c3cccc4ccccc34)cc2)c2ccccc2n1 ; CC1(C)CCC(C)(C)c2cc3c(cc21)c1c2c4c5ccccc5ccc4n(-c4nc5ccccc5nc4-c4ccc5cc(-c6ccccc6)ccc5c4)c2ccc1n3-c1ccccc1 ; CC1(C)CCC(C)(C)c2cc3c(cc21)c1c2c4c5ccccc5ccc4n(-c4ccccc4)c2ccc1n3-c1nc(-c2cccc(-c3cccc4ccccc34)c2)c2oc3ccccc3c2n1]

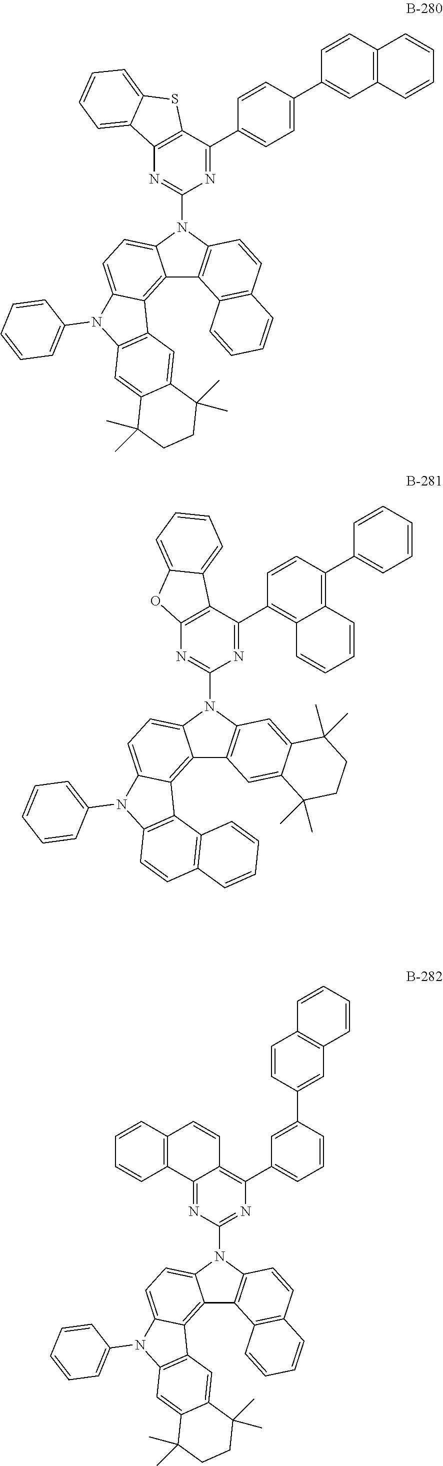 [SMILES: CC1(C)CCC(C)(C)c2cc3c(cc21)c1c2c4c5ccccc5ccc4n(-c4nc(-c5ccc(-c6ccc7ccccc7c6)cc5)c5sc6ccccc6c5n4)c2ccc1n3-c1ccccc1 ; CC1(C)CCC(C)(C)c2cc3c(cc21)c1c2c4c5ccccc5ccc4n(-c4ccccc4)c2ccc1n3-c1nc(-c2ccc(-c3ccccc3)c3ccccc23)c2c(n1)oc1ccccc12 ; CC1(C)CCC(C)(C)c2cc3c(cc21)c1c2c4c5ccccc5ccc4n(-c4nc(-c5cccc(-c6ccc7ccccc7c6)c5)c5ccc6ccccc6c5n4)c2ccc1n3-c1ccccc1]

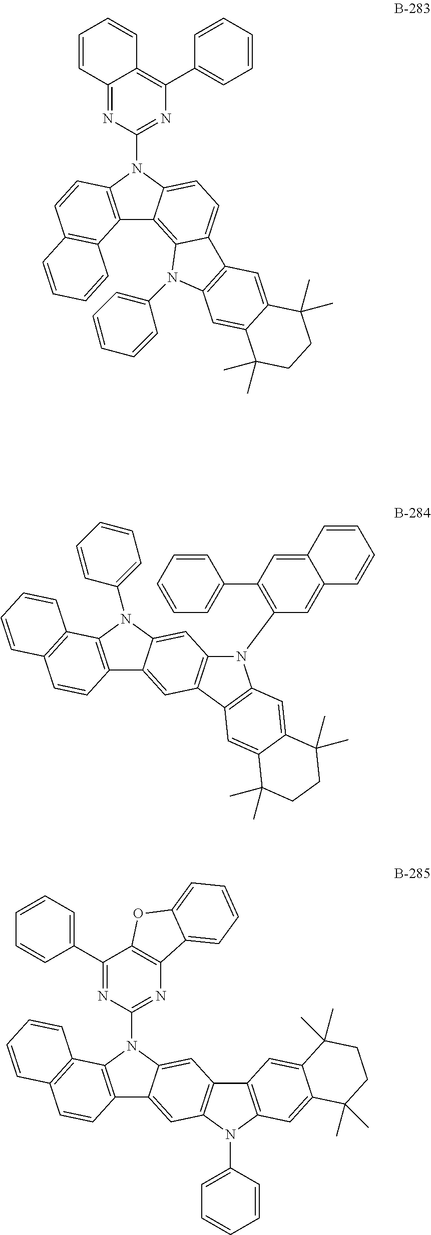 [SMILES: CC1(C)CCC(C)(C)c2cc3c(cc21)c1ccc2c(c4c5ccccc5ccc4n2-c2nc(-c4ccccc4)c4ccccc4n2)c1n3-c1ccccc1 ; CC1(C)CCC(C)(C)c2cc3c(cc21)c1cc2c4ccc5ccccc5c4n(-c4ccccc4)c2cc1n3-c1cc2ccccc2cc1-c1ccccc1 ; CC1(C)CCC(C)(C)c2cc3c(cc21)c1cc2c(cc1n3-c1ccccc1)c1ccc3ccccc3c1n2-c1nc(-c2ccccc2)c2oc3ccccc3c2n1]

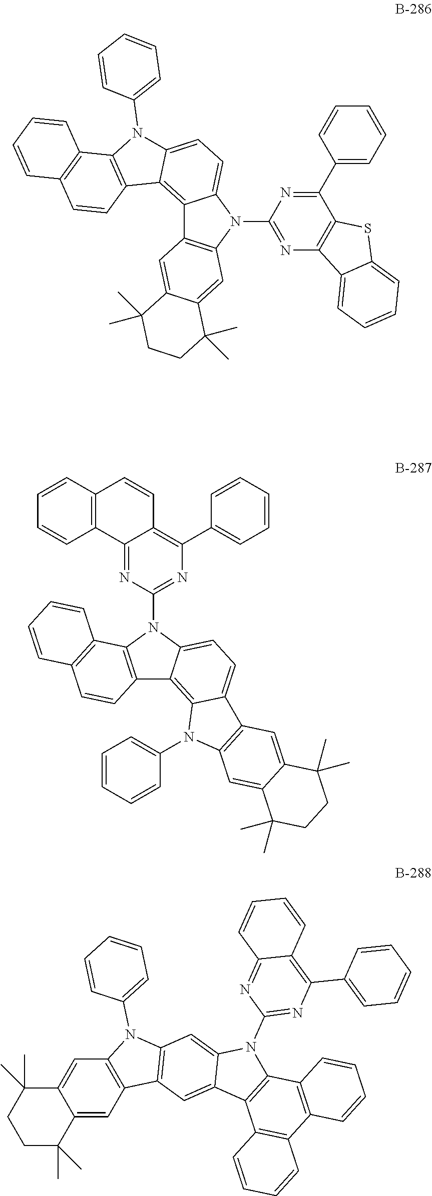

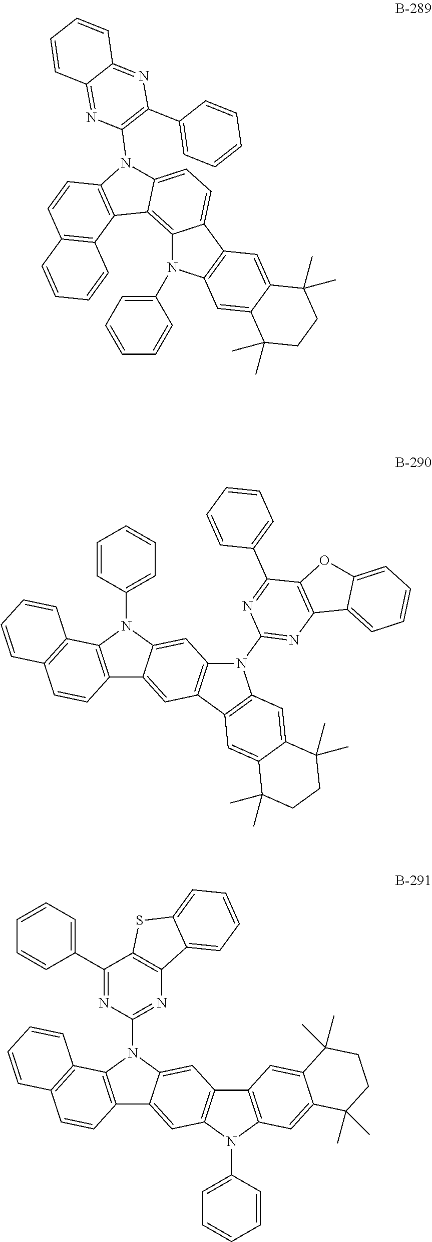 [SMILES: CC1(C)CCC(C)(C)c2cc3c(cc21)c1ccc2c(c4c5ccccc5ccc4n2-c2nc4ccccc4nc2-c2ccccc2)c1n3-c1ccccc1 ; CC1(C)CCC(C)(C)c2cc3c(cc21)c1cc2c4ccc5ccccc5c4n(-c4ccccc4)c2cc1n3-c1nc(-c2ccccc2)c2oc3ccccc3c2n1 ; CC1(C)CCC(C)(C)c2cc3c(cc21)c1cc2c(cc1n3-c1ccccc1)c1ccc3ccccc3c1n2-c1nc(-c2ccccc2)c2sc3ccccc3c2n1]

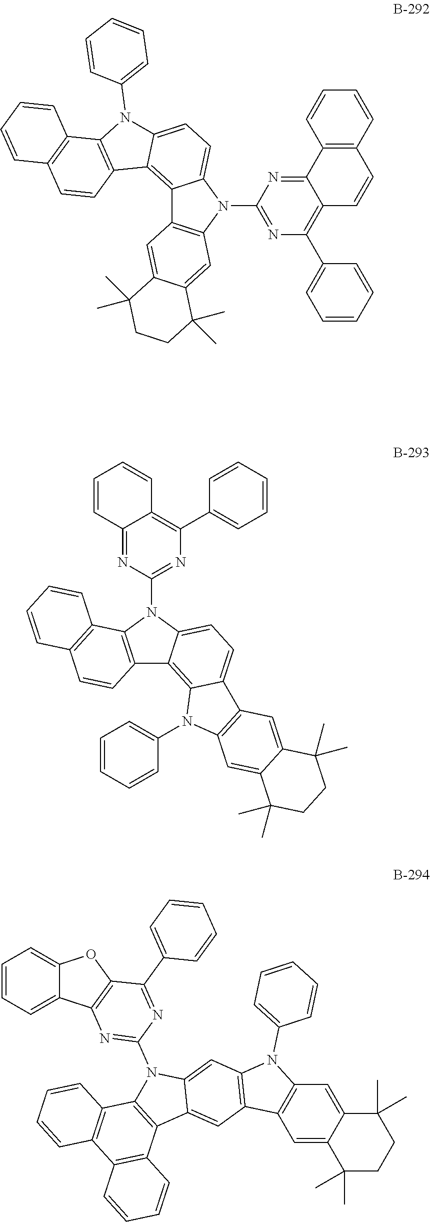 [SMILES: CC1(C)CCC(C)(C)c2cc3c(cc21)c1c2c4ccc5ccccc5c4n(-c4ccccc4)c2ccc1n3-c1nc(-c2ccccc2)c2ccc3ccccc3c2n1 ; CC1(C)CCC(C)(C)c2cc3c(cc21)c1ccc2c(c4ccc5ccccc5c4n2-c2nc(-c4ccccc4)c4ccccc4n2)c1n3-c1ccccc1 ; CC1(C)CCC(C)(C)c2cc3c(cc21)c1cc2c4c5ccccc5c5ccccc5c4n(-c4nc(-c5ccccc5)c5oc6ccccc6c5n4)c2cc1n3-c1ccccc1]

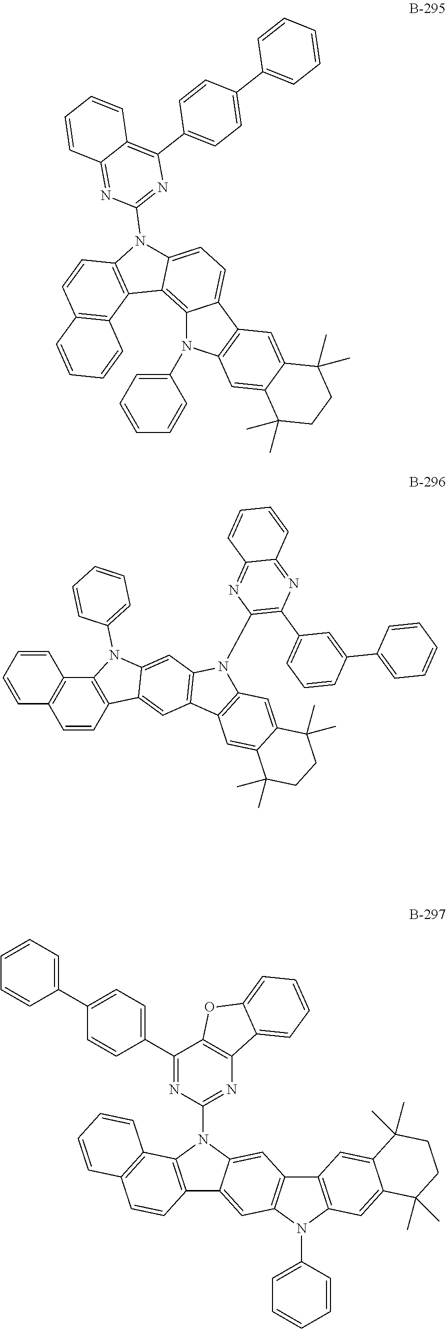 [SMILES: CC1(C)CCC(C)(C)c2cc3c(cc21)c1ccc2c(c4c5ccccc5ccc4n2-c2nc(-c4ccc(-c5ccccc5)cc4)c4ccccc4n2)c1n3-c1ccccc1 ; CC1(C)CCC(C)(C)c2cc3c(cc21)c1cc2c4ccc5ccccc5c4n(-c4ccccc4)c2cc1n3-c1nc2ccccc2nc1-c1cccc(-c2ccccc2)c1 ; CC1(C)CCC(C)(C)c2cc3c(cc21)c1cc2c(cc1n3-c1ccccc1)c1ccc3ccccc3c1n2-c1nc(-c2ccc(-c3ccccc3)cc2)c2oc3ccccc3c2n1]

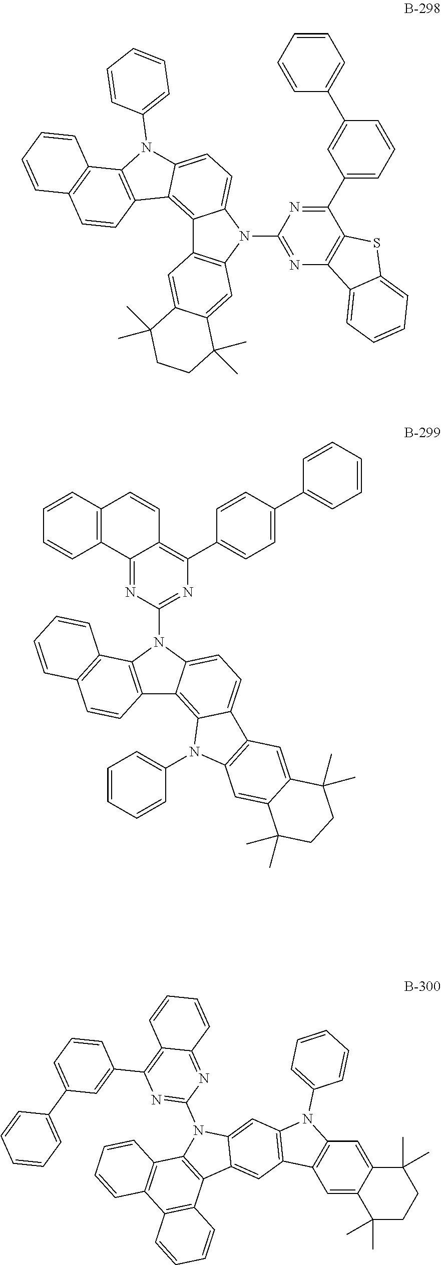 [SMILES: CC1(C)CCC(C)(C)c2cc3c(cc21)c1c2c4ccc5ccccc5c4n(-c4ccccc4)c2ccc1n3-c1nc(-c2cccc(-c3ccccc3)c2)c2sc3ccccc3c2n1 ; CC1(C)CCC(C)(C)c2cc3c(cc21)c1ccc2c(c4ccc5ccccc5c4n2-c2nc(-c4ccc(-c5ccccc5)cc4)c4ccc5ccccc5c4n2)c1n3-c1ccccc1 ; CC1(C)CCC(C)(C)c2cc3c(cc21)c1cc2c4c5ccccc5c5ccccc5c4n(-c4nc(-c5cccc(-c6ccccc6)c5)c5ccccc5n4)c2cc1n3-c1ccccc1]

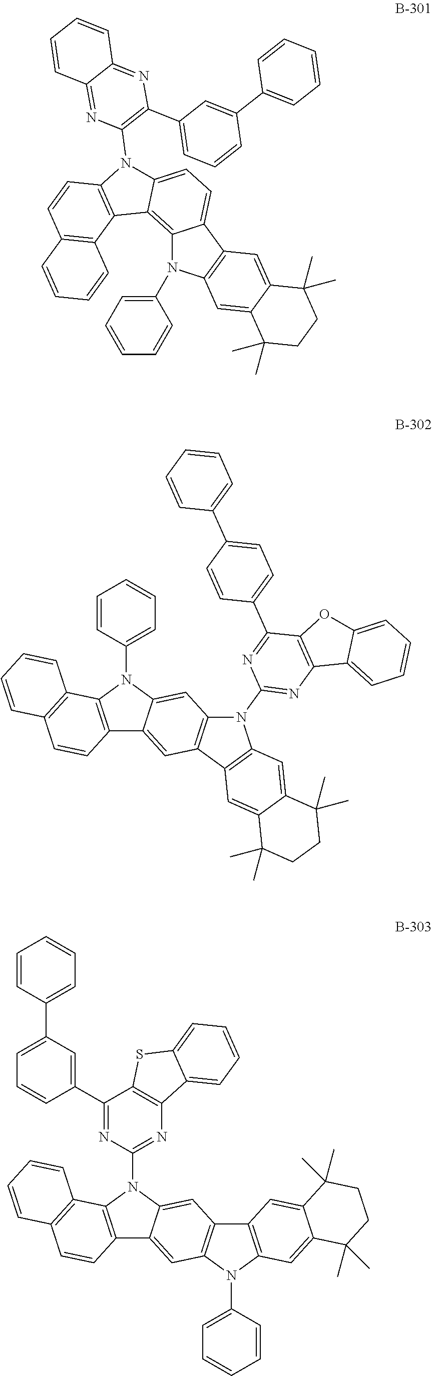 [SMILES: CC1(C)CCC(C)(C)c2cc3c(cc21)c1ccc2c(c4c5ccccc5ccc4n2-c2nc4ccccc4nc2-c2cccc(-c4ccccc4)c2)c1n3-c1ccccc1 ; CC1(C)CCC(C)(C)c2cc3c(cc21)c1cc2c4ccc5ccccc5c4n(-c4ccccc4)c2cc1n3-c1nc(-c2ccc(-c3ccccc3)cc2)c2oc3ccccc3c2n1 ; CC1(C)CCC(C)(C)c2cc3c(cc21)c1cc2c(cc1n3-c1ccccc1)c1ccc3ccccc3c1n2-c1nc(-c2cccc(-c3ccccc3)c2)c2sc3ccccc3c2n1]

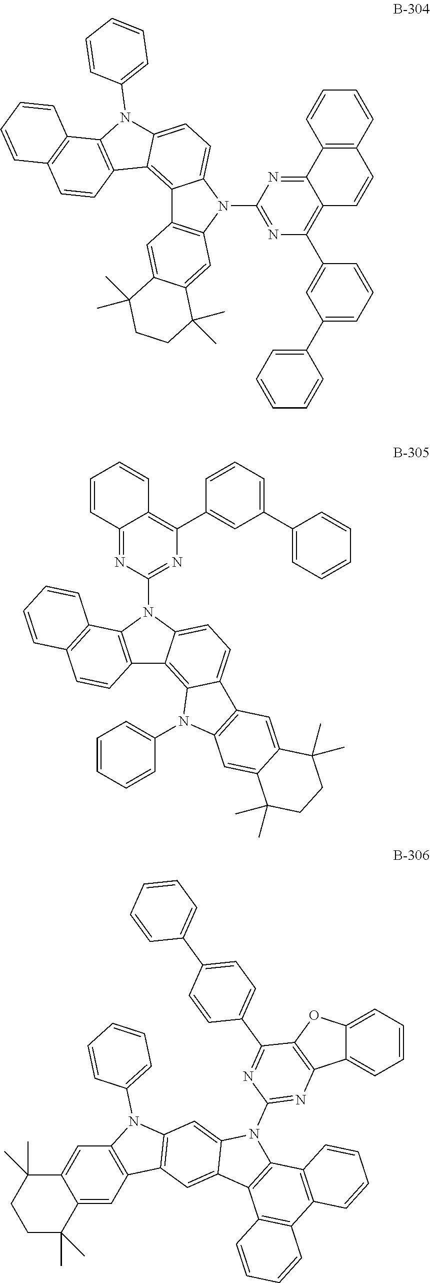 [SMILES: CC1(C)CCC(C)(C)c2cc3c(cc21)c1c2c4ccc5ccccc5c4n(-c4ccccc4)c2ccc1n3-c1nc(-c2cccc(-c3ccccc3)c2)c2ccc3ccccc3c2n1 ; CC1(C)CCC(C)(C)c2cc3c(cc21)c1ccc2c(c4ccc5ccccc5c4n2-c2nc(-c4cccc(-c5ccccc5)c4)c4ccccc4n2)c1n3-c1ccccc1 ; CC1(C)CCC(C)(C)c2cc3c(cc21)c1cc2c4c5ccccc5c5ccccc5c4n(-c4nc(-c5ccc(-c6ccccc6)cc5)c5oc6ccccc6c5n4)c2cc1n3-c1ccccc1]

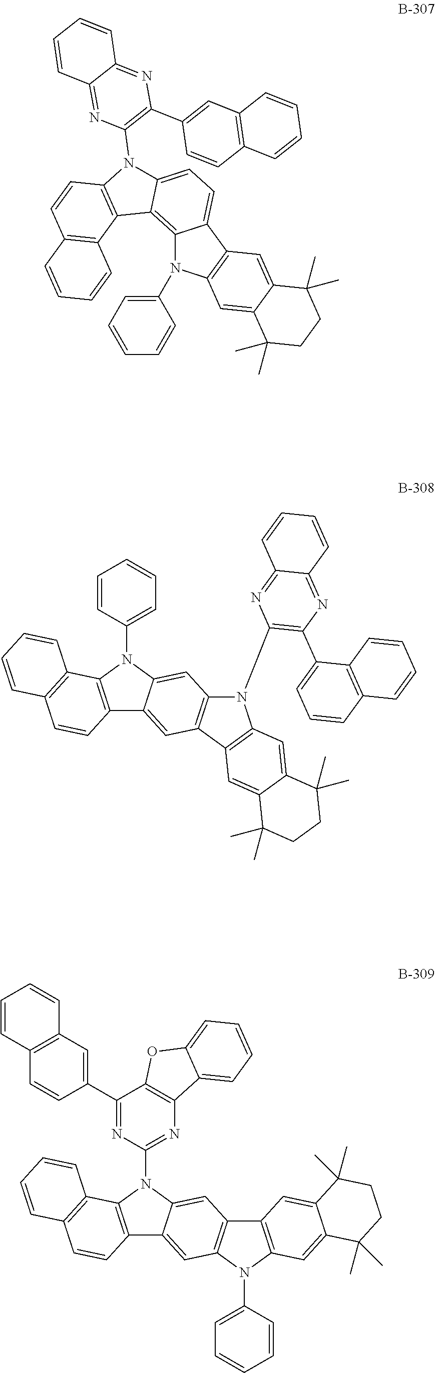 [SMILES: CC1(C)CCC(C)(C)c2cc3c(cc21)c1ccc2c(c4c5ccccc5ccc4n2-c2nc4ccccc4nc2-c2ccc4ccccc4c2)c1n3-c1ccccc1 ; CC1(C)CCC(C)(C)c2cc3c(cc21)c1cc2c4ccc5ccccc5c4n(-c4ccccc4)c2cc1n3-c1nc2ccccc2nc1-c1cccc2ccccc12 ; CC1(C)CCC(C)(C)c2cc3c(cc21)c1cc2c(cc1n3-c1ccccc1)c1ccc3ccccc3c1n2-c1nc(-c2ccc3ccccc3c2)c2oc3ccccc3c2n1]

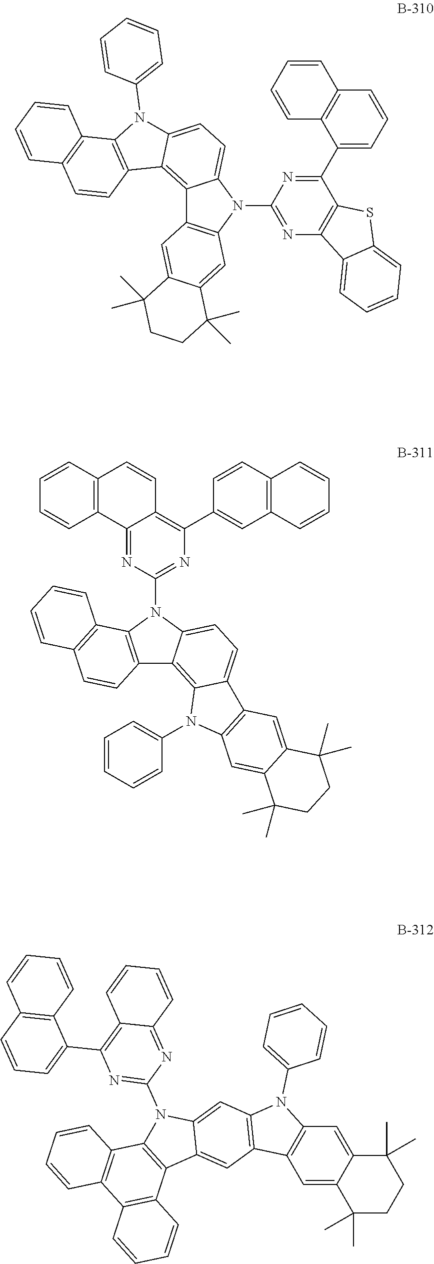 [SMILES: CC1(C)CCC(C)(C)c2cc3c(cc21)c1c2c4ccc5ccccc5c4n(-c4ccccc4)c2ccc1n3-c1nc(-c2cccc3ccccc23)c2sc3ccccc3c2n1 ; CC1(C)CCC(C)(C)c2cc3c(cc21)c1ccc2c(c4ccc5ccccc5c4n2-c2nc(-c4ccc5ccccc5c4)c4ccc5ccccc5c4n2)c1n3-c1ccccc1 ; CC1(C)CCC(C)(C)c2cc3c(cc21)c1cc2c4c5ccccc5c5ccccc5c4n(-c4nc(-c5cccc6ccccc56)c5ccccc5n4)c2cc1n3-c1ccccc1]

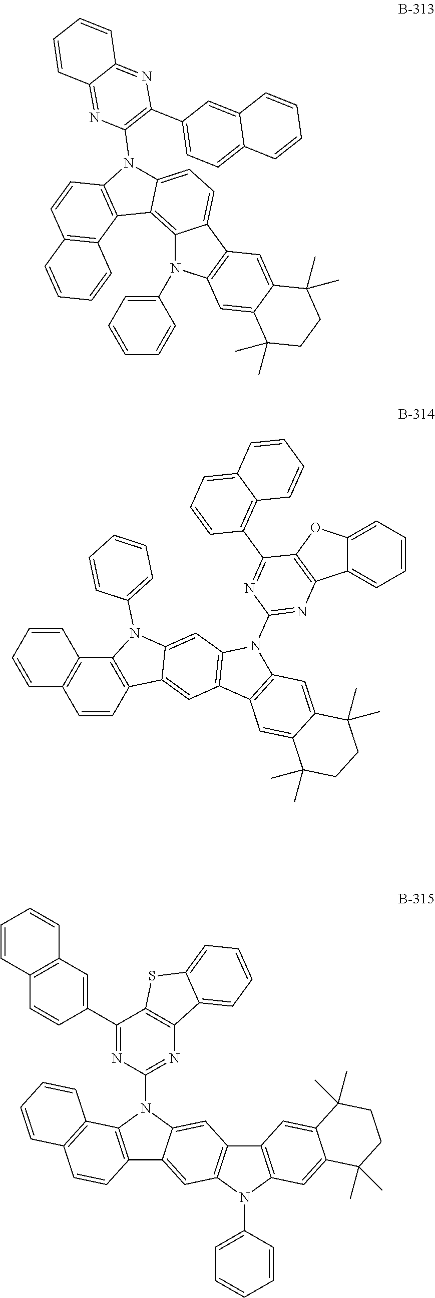 [SMILES: CC1(C)CCC(C)(C)c2cc3c(cc21)c1ccc2c(c4c5ccccc5ccc4n2-c2nc4ccccc4nc2-c2ccc4ccccc4c2)c1n3-c1ccccc1 ; CC1(C)CCC(C)(C)c2cc3c(cc21)c1cc2c4ccc5ccccc5c4n(-c4ccccc4)c2cc1n3-c1nc(-c2cccc3ccccc23)c2oc3ccccc3c2n1 ; CC1(C)CCC(C)(C)c2cc3c(cc21)c1cc2c(cc1n3-c1ccccc1)c1ccc3ccccc3c1n2-c1nc(-c2ccc3ccccc3c2)c2sc3ccccc3c2n1]

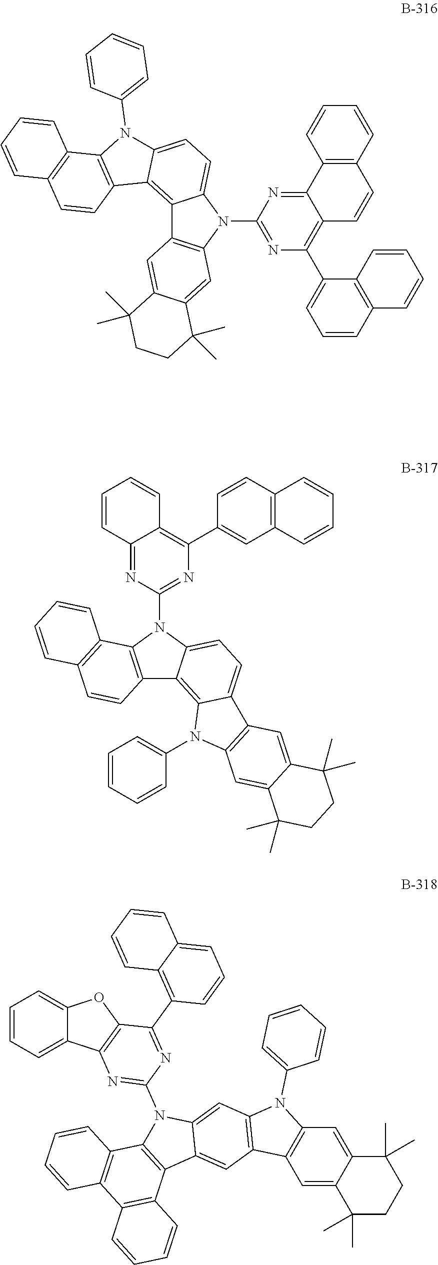 [SMILES: CC1(C)CCC(C)(C)c2cc3c(cc21)c1c2c4ccc5ccccc5c4n(-c4ccccc4)c2ccc1n3-c1nc(-c2cccc3ccccc23)c2ccc3ccccc3c2n1 ; CC1(C)CCC(C)(C)c2cc3c(cc21)c1ccc2c(c4ccc5ccccc5c4n2-c2nc(-c4ccc5ccccc5c4)c4ccccc4n2)c1n3-c1ccccc1 ; CC1(C)CCC(C)(C)c2cc3c(cc21)c1cc2c4c5ccccc5c5ccccc5c4n(-c4nc(-c5cccc6ccccc56)c5oc6ccccc6c5n4)c2cc1n3-c1ccccc1]

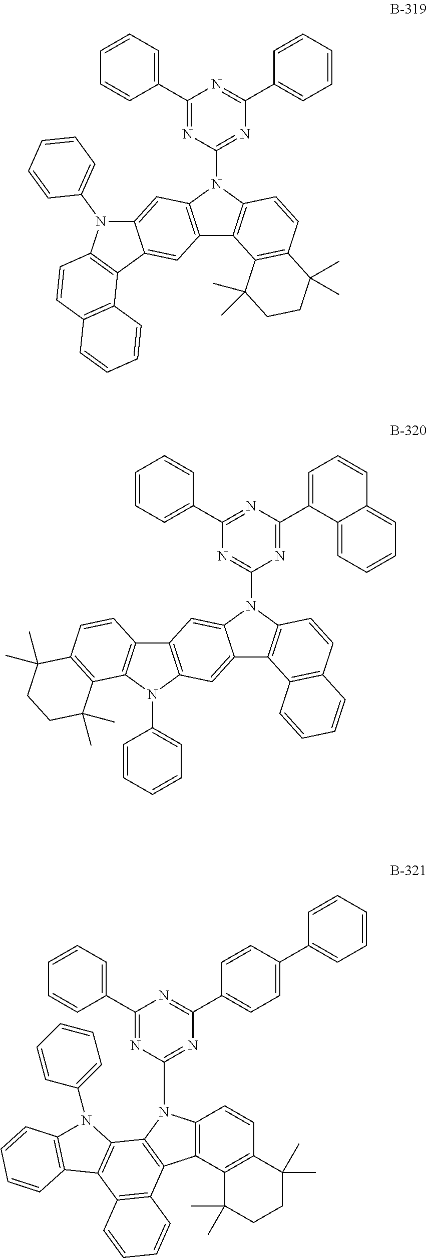

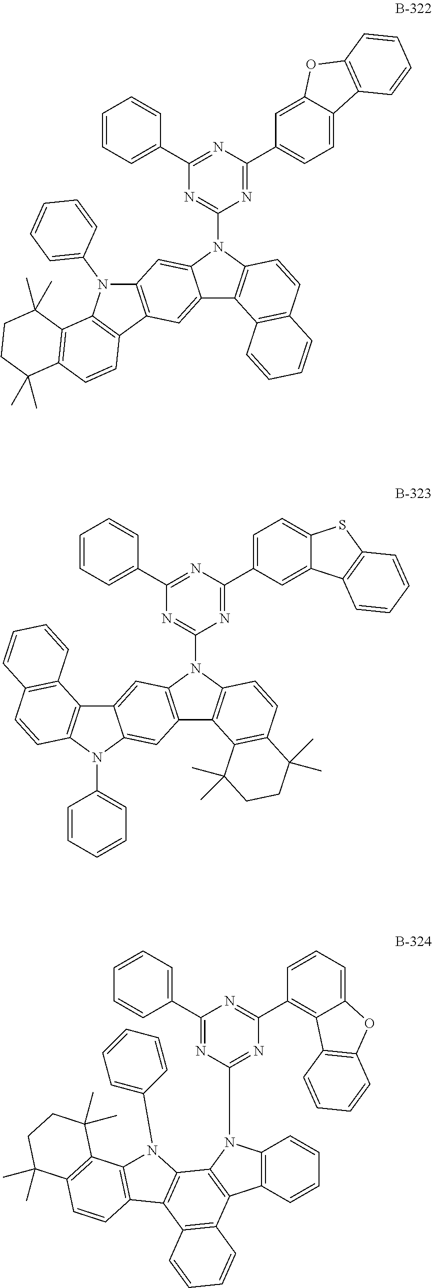 [SMILES: CC1(C)CCC(C)(C)c2c1ccc1c3cc4c5c6ccccc6ccc5n(-c5nc(-c6ccccc6)nc(-c6ccc7c(c6)oc6ccccc67)n5)c4cc3n(-c3ccccc3)c21 ; CC1(C)CCC(C)(C)c2c1ccc1c2c2cc3c(cc2n1-c1nc(-c2ccccc2)nc(-c2ccc4sc5ccccc5c4c2)n1)c1c2ccccc2ccc1n3-c1ccccc1 ; CC1(C)CCC(C)(C)c2c1ccc1c3c4ccccc4c4c5ccccc5n(-c5nc(-c6ccccc6)nc(-c6cccc7oc8ccccc8c67)n5)c4c3n(-c3ccccc3)c21]

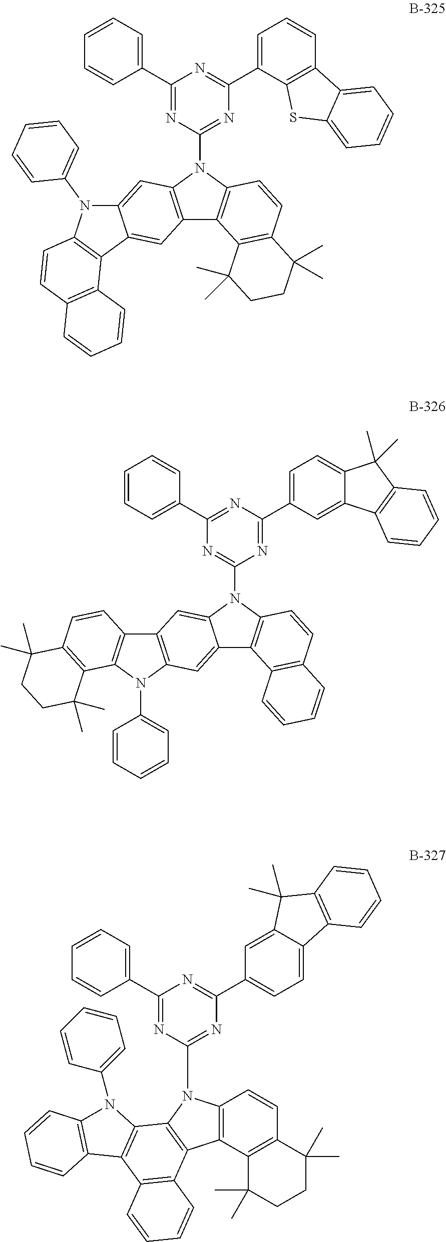 [SMILES: CC1(C)CCC(C)(C)c2c1ccc1c2c2cc3c4c5ccccc5ccc4n(-c4ccccc4)c3cc2n1-c1nc(-c2ccccc2)nc(-c2cccc3c2sc2ccccc23)n1 ; CC1(C)CCC(C)(C)c2c1ccc1c3cc4c(cc3n(-c3ccccc3)c21)c1c2ccccc2ccc1n4-c1nc(-c2ccccc2)nc(-c2ccc3c(c2)-c2ccccc2C3(C)C)n1 ; CC1(C)CCC(C)(C)c2c1ccc1c2c2c3ccccc3c3c4ccccc4n(-c4ccccc4)c3c2n1-c1nc(-c2ccccc2)nc(-c2ccc3c(c2)C(C)(C)c2ccccc2-3)n1]

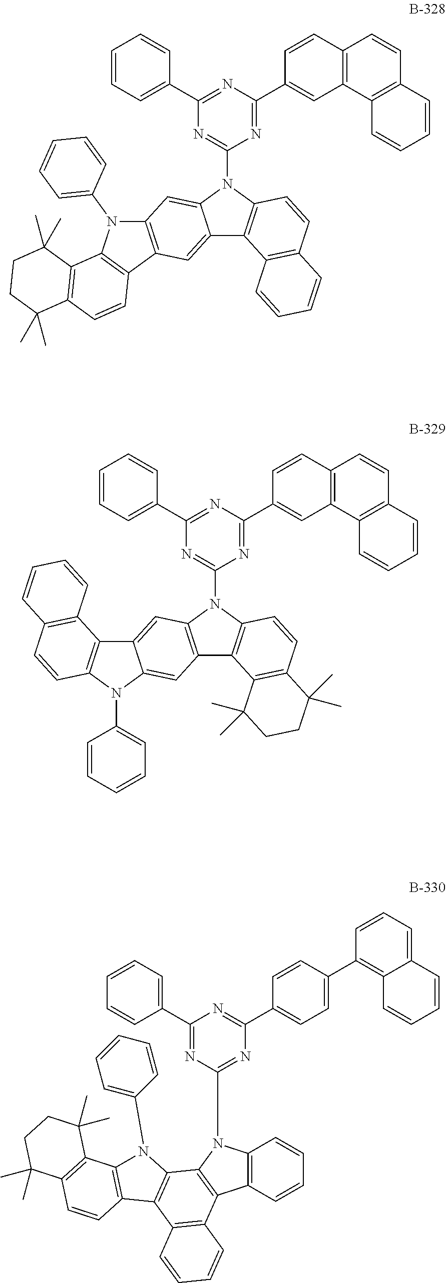 [SMILES: CC1(C)CCC(C)(C)c2c1ccc1c3cc4c5c6ccccc6ccc5n(-c5nc(-c6ccccc6)nc(-c6ccc7ccc8ccccc8c7c6)n5)c4cc3n(-c3ccccc3)c21 ; CC1(C)CCC(C)(C)c2c1ccc1c2c2cc3c(cc2n1-c1nc(-c2ccccc2)nc(-c2ccc4ccc5ccccc5c4c2)n1)c1c2ccccc2ccc1n3-c1ccccc1 ; CC1(C)CCC(C)(C)c2c1ccc1c3c4ccccc4c4c5ccccc5n(-c5nc(-c6ccccc6)nc(-c6ccc(-c7cccc8ccccc78)cc6)n5)c4c3n(-c3ccccc3)c21]

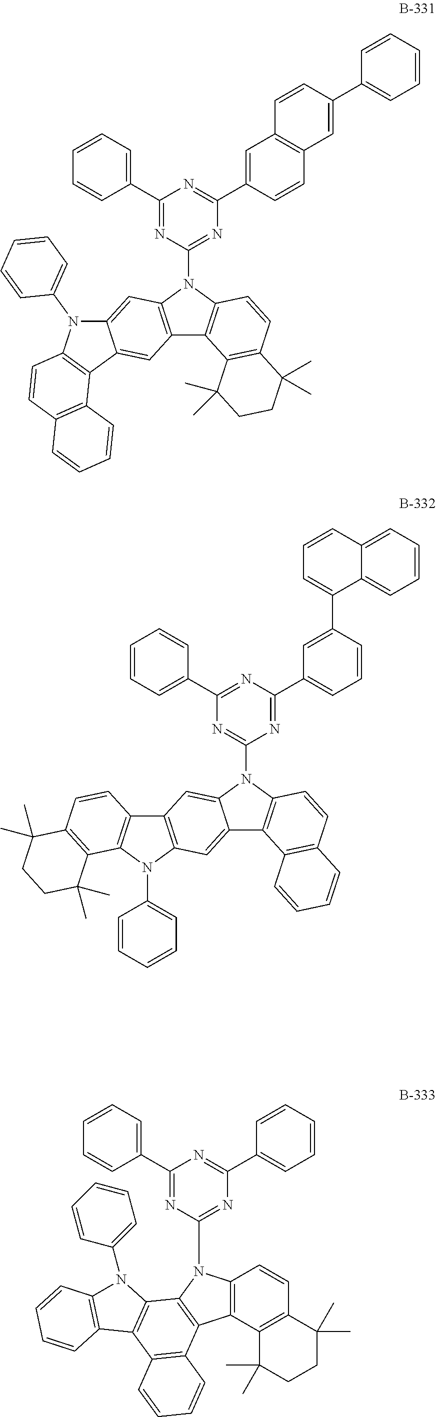 [SMILES: CC1(C)CCC(C)(C)c2c1ccc1c2c2cc3c4c5ccccc5ccc4n(-c4ccccc4)c3cc2n1-c1nc(-c2ccccc2)nc(-c2ccc3cc(-c4ccccc4)ccc3c2)n1 ; CC1(C)CCC(C)(C)c2c1ccc1c3cc4c(cc3n(-c3ccccc3)c21)c1c2ccccc2ccc1n4-c1nc(-c2ccccc2)nc(-c2cccc(-c3cccc4ccccc34)c2)n1 ; CC1(C)CCC(C)(C)c2c1ccc1c2c2c3ccccc3c3c4ccccc4n(-c4ccccc4)c3c2n1-c1nc(-c2ccccc2)nc(-c2ccccc2)n1]

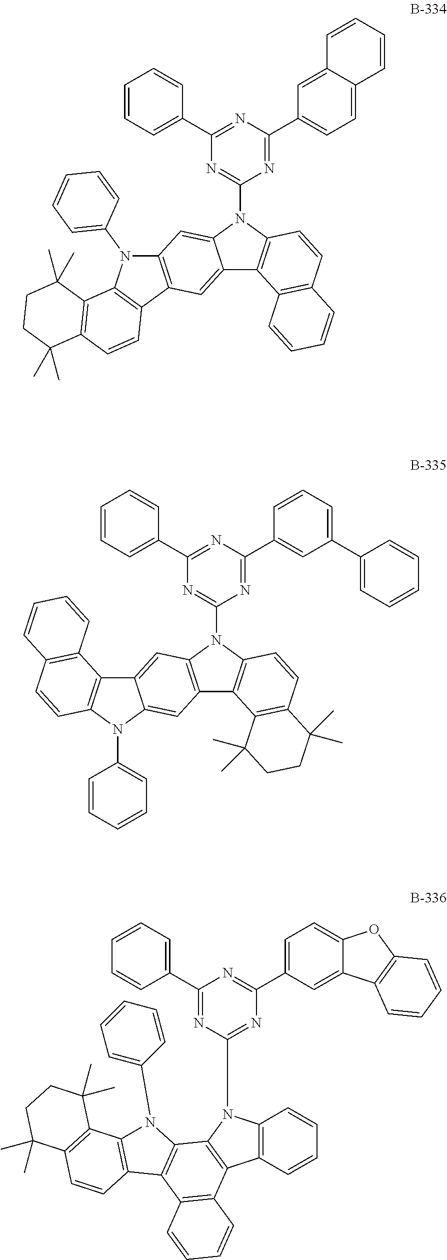 [SMILES: CC1(C)CCC(C)(C)c2c1ccc1c3cc4c5c6ccccc6ccc5n(-c5nc(-c6ccccc6)nc(-c6ccc7ccccc7c6)n5)c4cc3n(-c3ccccc3)c21 ; CC1(C)CCC(C)(C)c2c1ccc1c2c2cc3c(cc2n1-c1nc(-c2ccccc2)nc(-c2cccc(-c4ccccc4)c2)n1)c1c2ccccc2ccc1n3-c1ccccc1 ; CC1(C)CCC(C)(C)c2c1ccc1c3c4ccccc4c4c5ccccc5n(-c5nc(-c6ccccc6)nc(-c6ccc7oc8ccccc8c7c6)n5)c4c3n(-c3ccccc3)c21]

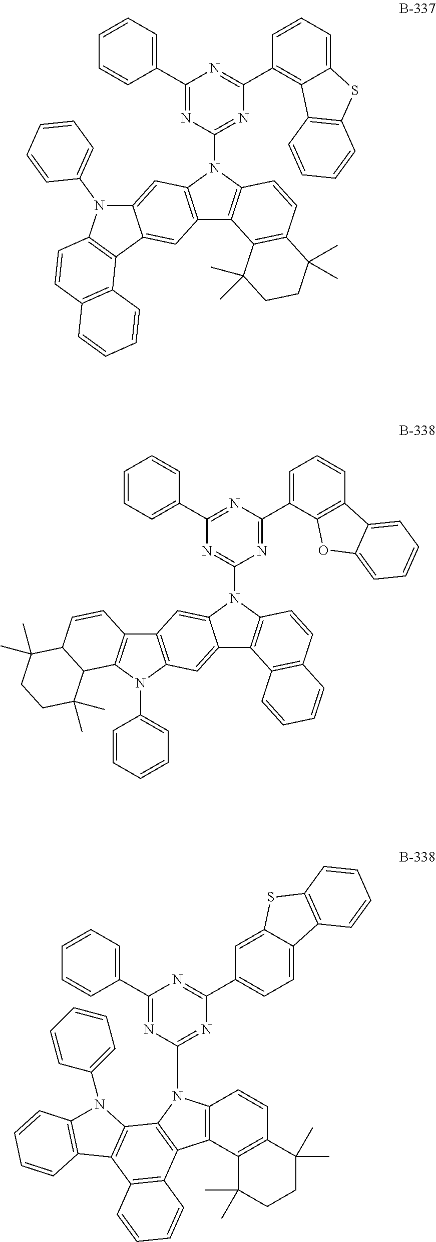 [SMILES: CC1(C)CCC(C)(C)c2c1ccc1c2c2cc3c4c5ccccc5ccc4n(-c4ccccc4)c3cc2n1-c1nc(-c2ccccc2)nc(-c2cccc3sc4ccccc4c23)n1 ; CC1(C)CCC(C)(C)C2c3c(c4cc5c(cc4n3-c3ccccc3)c3c4ccccc4ccc3n5-c3nc(-c4ccccc4)nc(-c4cccc5c4oc4ccccc45)n3)C=CC21 ; CC1(C)CCC(C)(C)c2c1ccc1c2c2c3ccccc3c3c4ccccc4n(-c4ccccc4)c3c2n1-c1nc(-c2ccccc2)nc(-c2ccc3c(c2)sc2ccccc23)n1]

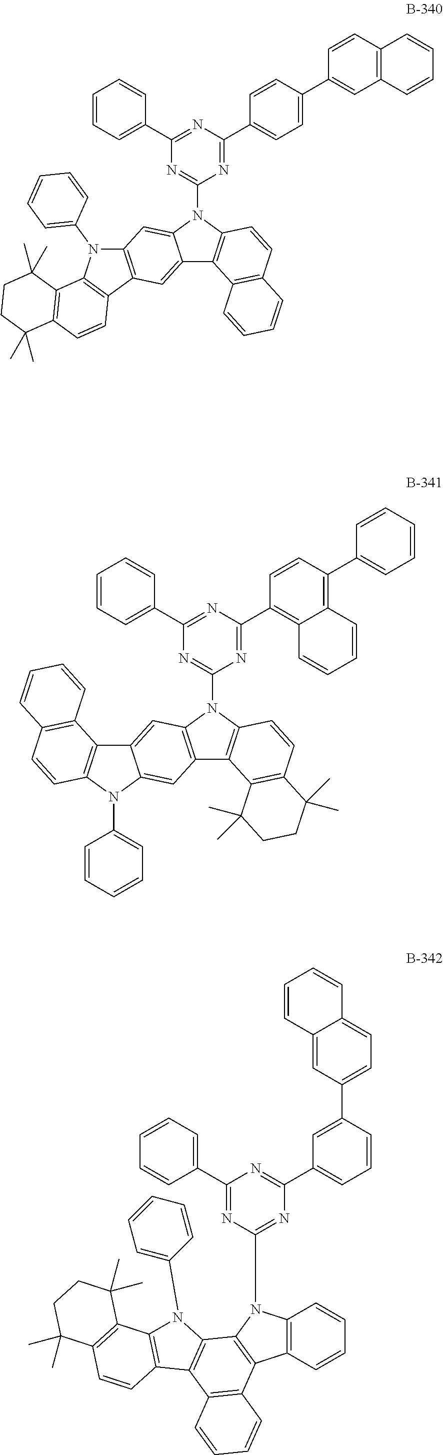 [SMILES: CC1(C)CCC(C)(C)c2c1ccc1c3cc4c5c6ccccc6ccc5n(-c5nc(-c6ccccc6)nc(-c6ccc(-c7ccc8ccccc8c7)cc6)n5)c4cc3n(-c3ccccc3)c21 ; CC1(C)CCC(C)(C)c2c1ccc1c2c2cc3c(cc2n1-c1nc(-c2ccccc2)nc(-c2ccc(-c4ccccc4)c4ccccc24)n1)c1c2ccccc2ccc1n3-c1ccccc1 ; CC1(C)CCC(C)(C)c2c1ccc1c3c4ccccc4c4c5ccccc5n(-c5nc(-c6ccccc6)nc(-c6cccc(-c7ccc8ccccc8c7)c6)n5)c4c3n(-c3ccccc3)c21]

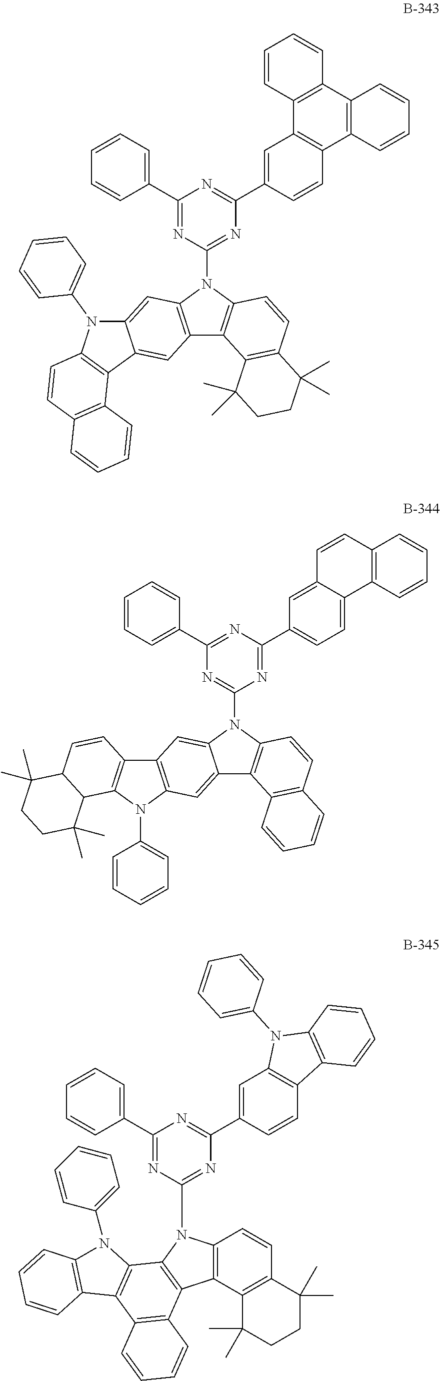 [SMILES: CC1(C)CCC(C)(C)c2c1ccc1c2c2cc3c4c5ccccc5ccc4n(-c4ccccc4)c3cc2n1-c1nc(-c2ccccc2)nc(-c2ccc3c4ccccc4c4ccccc4c3c2)n1 ; CC1(C)CCC(C)(C)C2c3c(c4cc5c(cc4n3-c3ccccc3)c3c4ccccc4ccc3n5-c3nc(-c4ccccc4)nc(-c4ccc5c(ccc6ccccc65)c4)n3)C=CC21 ; CC1(C)CCC(C)(C)c2c1ccc1c2c2c3ccccc3c3c4ccccc4n(-c4ccccc4)c3c2n1-c1nc(-c2ccccc2)nc(-c2ccc3c4ccccc4n(-c4ccccc4)c3c2)n1]

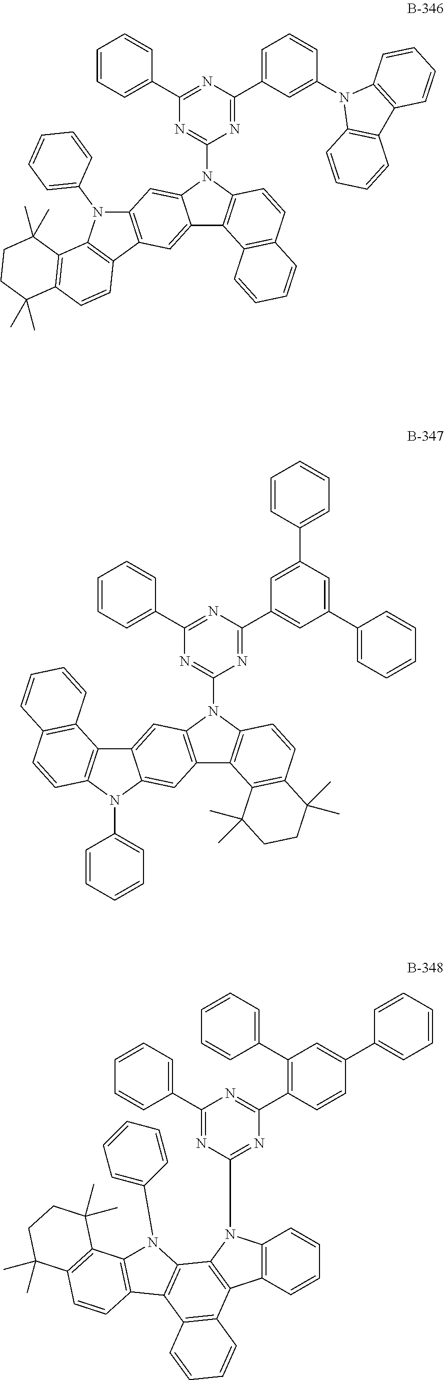 [SMILES: CC1(C)CCC(C)(C)c2c1ccc1c3cc4c5c6ccccc6ccc5n(-c5nc(-c6ccccc6)nc(-c6cccc(-n7c8ccccc8c8ccccc87)c6)n5)c4cc3n(-c3ccccc3)c21 ; CC1(C)CCC(C)(C)c2c1ccc1c2c2cc3c(cc2n1-c1nc(-c2ccccc2)nc(-c2cc(-c4ccccc4)cc(-c4ccccc4)c2)n1)c1c2ccccc2ccc1n3-c1ccccc1 ; CC1(C)CCC(C)(C)c2c1ccc1c3c4ccccc4c4c5ccccc5n(-c5nc(-c6ccccc6)nc(-c6ccc(-c7ccccc7)cc6-c6ccccc6)n5)c4c3n(-c3ccccc3)c21]

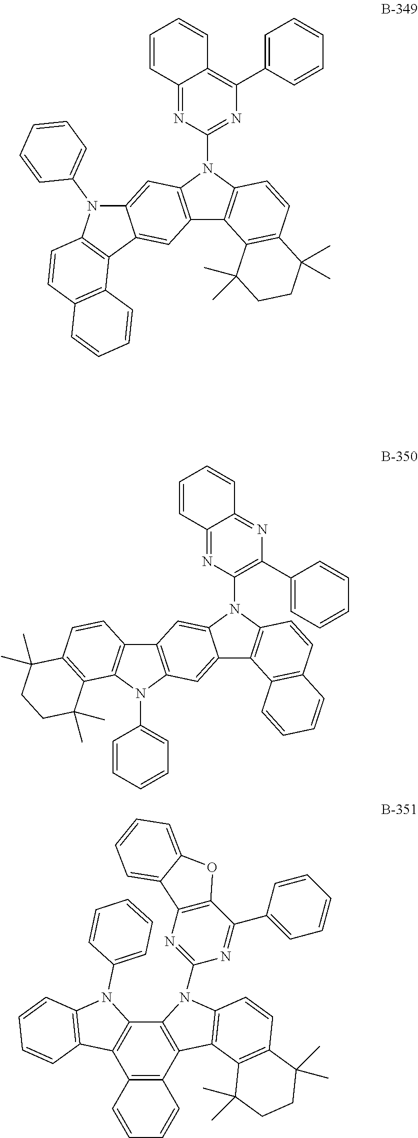

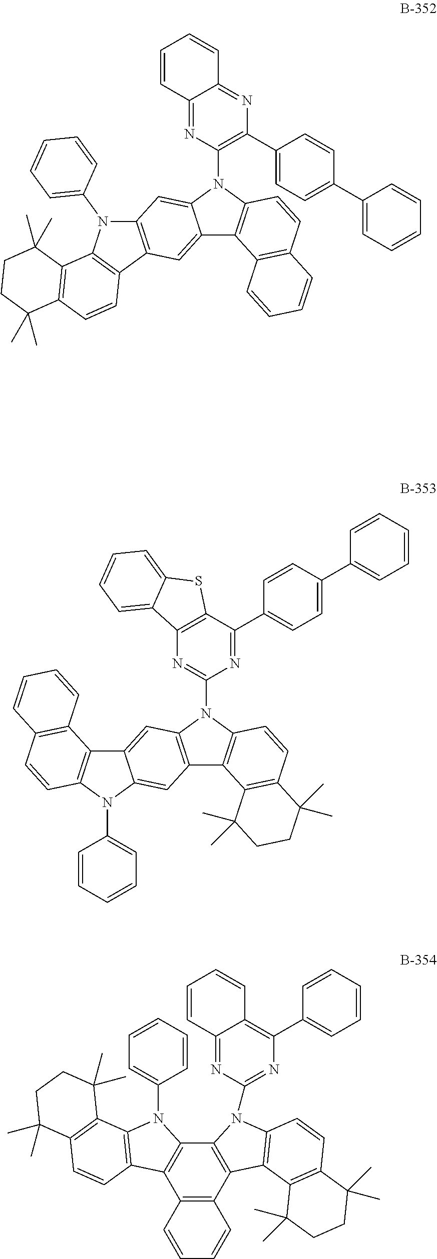

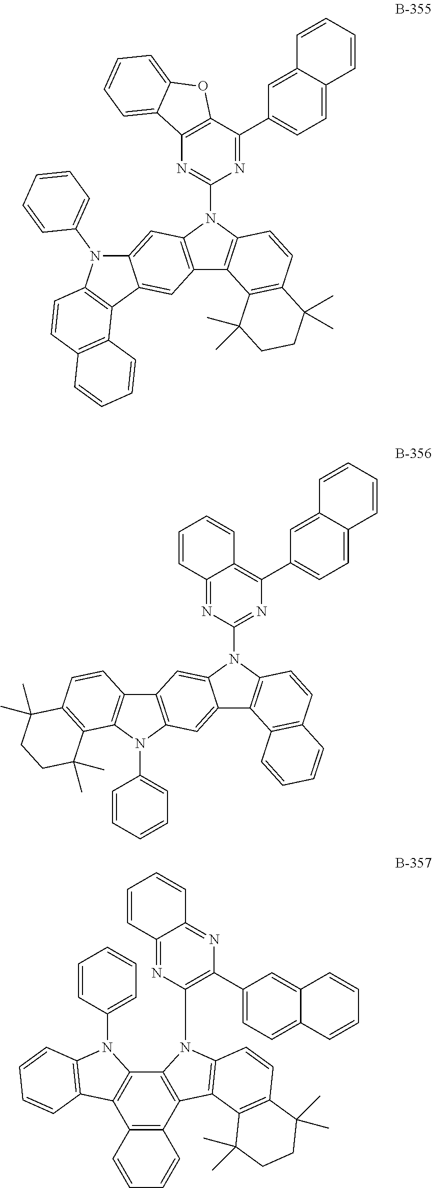

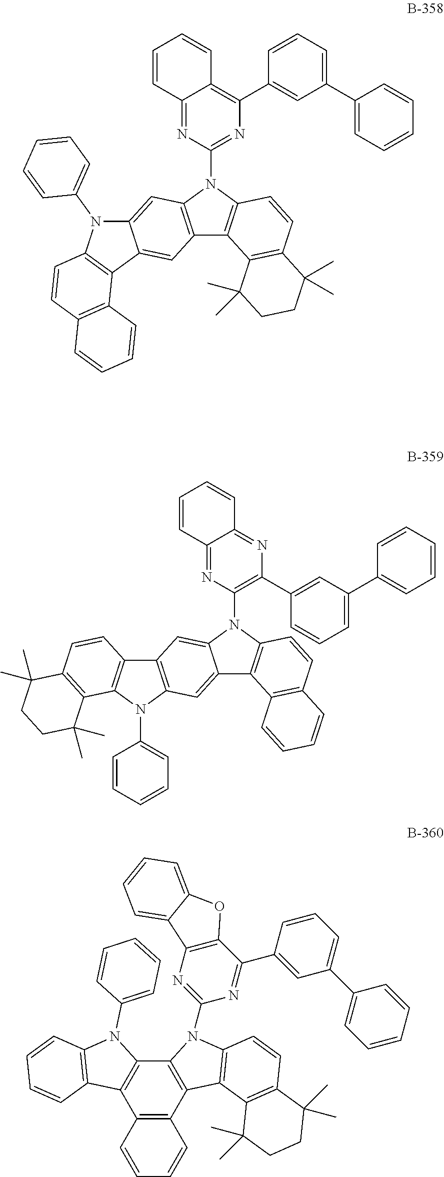 [SMILES: CC1(C)CCC(C)(C)c2c1ccc1c2c2cc3c4c5ccccc5ccc4n(-c4ccccc4)c3cc2n1-c1nc(-c2cccc(-c3ccccc3)c2)c2ccccc2n1 ; CC1(C)CCC(C)(C)c2c1ccc1c3cc4c(cc3n(-c3ccccc3)c21)c1c2ccccc2ccc1n4-c1nc2ccccc2nc1-c1cccc(-c2ccccc2)c1 ; CC1(C)CCC(C)(C)c2c1ccc1c2c2c3ccccc3c3c4ccccc4n(-c4ccccc4)c3c2n1-c1nc(-c2cccc(-c3ccccc3)c2)c2oc3ccccc3c2n1]

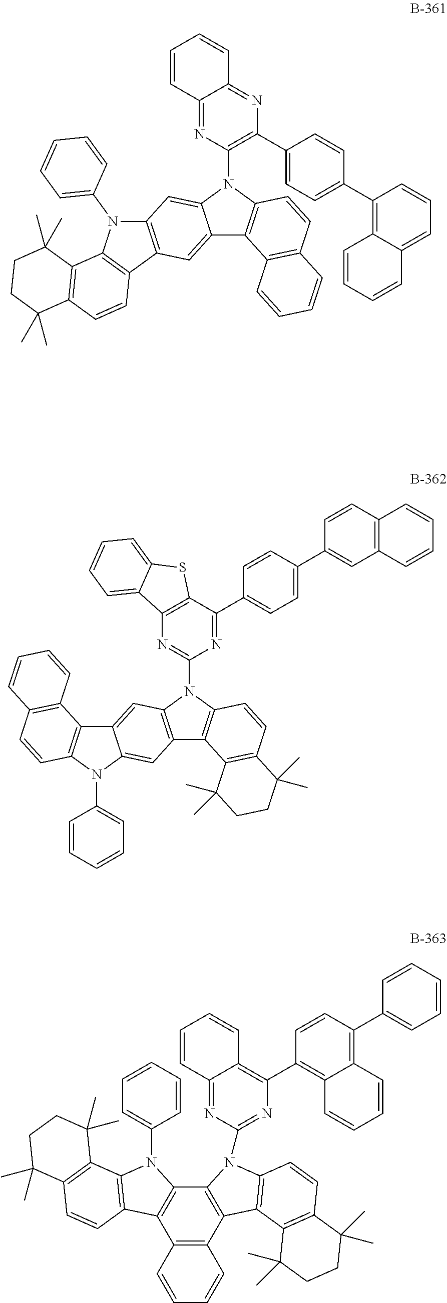 [SMILES: CC1(C)CCC(C)(C)c2c1ccc1c3cc4c5c6ccccc6ccc5n(-c5nc6ccccc6nc5-c5ccc(-c6cccc7ccccc67)cc5)c4cc3n(-c3ccccc3)c21 ; CC1(C)CCC(C)(C)c2c1ccc1c2c2cc3c(cc2n1-c1nc(-c2ccc(-c4ccc5ccccc5c4)cc2)c2sc4ccccc4c2n1)c1c2ccccc2ccc1n3-c1ccccc1 ; CC1(C)CCC(C)(C)c2c1ccc1c2c2c3ccccc3c3c4ccc5c(c4n(-c4ccccc4)c3c2n1-c1nc(-c2ccc(-c3ccccc3)c3ccccc23)c2ccccc2n1)C(C)(C)CCC5(C)C]

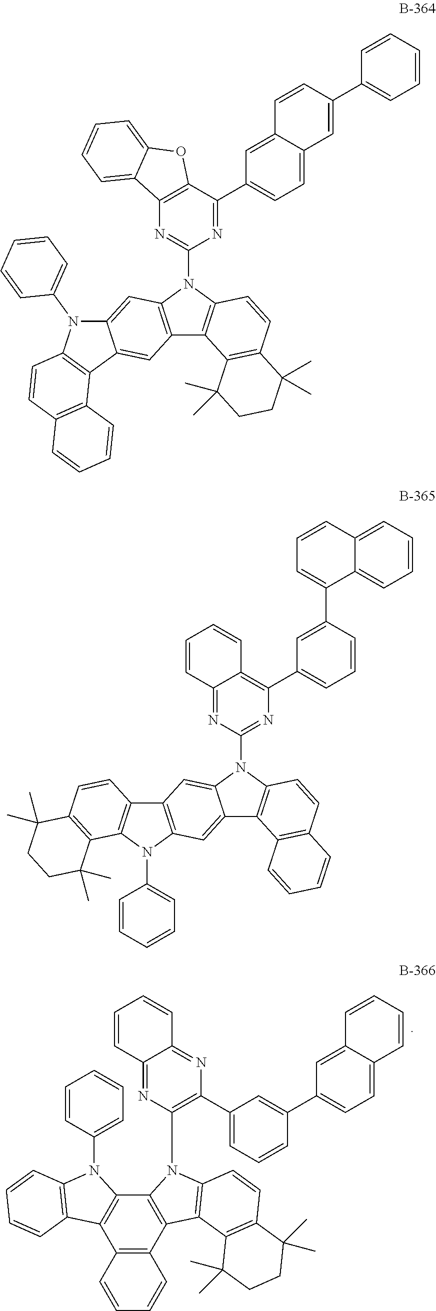 [SMILES: CC1(C)CCC(C)(C)c2c1ccc1c2c2cc3c4c5ccccc5ccc4n(-c4ccccc4)c3cc2n1-c1nc(-c2ccc3cc(-c4ccccc4)ccc3c2)c2oc3ccccc3c2n1 ; CC1(C)CCC(C)(C)c2c1ccc1c3cc4c(cc3n(-c3ccccc3)c21)c1c2ccccc2ccc1n4-c1nc(-c2cccc(-c3cccc4ccccc34)c2)c2ccccc2n1 ; CC1(C)CCC(C)(C)c2c1ccc1c2c2c3ccccc3c3c4ccccc4n(-c4ccccc4)c3c2n1-c1nc2ccccc2nc1-c1cccc(-c2ccc3ccccc3c2)c1]

13. An organic electroluminescent device, comprising an anode and a cathode that are disposed facing each other, and a functional layer disposed between the anode and the cathode, wherein the functional layer comprises the nitrogen-containing compound according to claim 1;
alternatively, the functional layer comprises an organic light emitting layer, the organic light emitting layer comprising the nitrogen-containing compound.

14. An electronic apparatus, comprising the organic electroluminescent device according to claim 13.
